SEQUENCE LISTING (83 sequences)

SEQ ID NO: 1 - RNA, 22 nt, Artificial Sequence
  Synthetic miR-150-5p
ucucccaacccuuguaccagug

SEQ ID NO: 2 - DNA, 22 bp, Artificial Sequence
  Synthetic miR-150-5p binding site
cactggtacaagggttgggaga

SEQ ID NO: 3 - RNA, 23 nt, Artificial Sequence
  Synthetic miR-146b-5p
ugagaacugaauuccauaggcug

SEQ ID NO: 4 - DNA, 23 bp, Artificial Sequence
  Synthetic miR-146b-5p binding site
cagcctatggaattcagttctca

SEQ ID NO: 5 - RNA, 17 nt, Artificial Sequence
  Synthetic miR 4286
accccacuccugguacc

SEQ ID NO: 6 - DNA, 17 bp, Artificial Sequence
  Synthetic miR-4286 binding site
ggtaccaggagtggggt

SEQ ID NO: 7 - RNA, 23 nt, Artificial Sequence
  Synthetic miR-579-3b
uucauuugguauaaaccgcgauu

SEQ ID NO: 8 - DNA, 23 bp, Artificial Sequence
  Synthetic miR-579-3b binding site
aatcgcggtttataccaaatgaa

SEQ ID NO: 9 - RNA, 17 nt, Artificial Sequence
  Synthetic miR-4516
gggagaagggucggggc

SEQ ID NO: 10 - DNA, 17 bp, Artificial Sequence
  Synthetic miR-4516 binding site
gccccgacccttctccc

SEQ ID NO: 11 - RNA, 22 nt, Artificial Sequence
  Synthetic miR-146a-5p
ugagaacugaauuccauggguu

SEQ ID NO: 12 - DNA, 22 bp, Artificial Sequence
  Synthetic miR-146a-5p binding site
aacccatggaattcagttctca

SEQ ID NO: 13 - RNA, 22 nt, Artificial Sequence
  Synthetic miR-664b-3p
uucauuugccucccagccuaca

SEQ ID NO: 14 - DNA, 22 bp, Artificial Sequence
  Synthetic miR-664b-3p binding site
tgtaggctgggaggcaaatgaa

SEQ ID NO: 15 - RNA, 23 nt, Artificial Sequence
  Synthetic miR-342-3p
ucucacacagaaaucgcacccgu

SEQ ID NO: 16 - DNA, 23 bp, Artificial Sequence
  Synthetic miR-342-3p binding site
acgggtgcgatttctgtgtgaga

SEQ ID NO: 17 - RNA, 21 nt, Artificial Sequence
  Synthetic miR-342-5p
aggggugcuaucugugauuga

SEQ ID NO: 18 - DNA, 21 bp, Artificial Sequence
  Synthetic miR-342-5p binding site
tcaatcacagatagcacccct

SEQ ID NO: 19 - DNA, 20 bp, Artificial Sequence
  Synthetic miR-1915-3p
ccccagggcgacgcggcggg

SEQ ID NO: 20 - DNA, 20 bp, Artificial Sequence
  Synthetic miR-1915-3p binding site
cccgccgcgtcgccctgggg

SEQ ID NO: 21 - RNA, 21 nt, Artificial Sequence
  Synthetic miR-26b-5p
uucaaguaauucaggauaggu

SEQ ID NO: 22 - DNA, 21 bp, Artificial Sequence
  Synthetic miR-26b-5p binding site
acctatcctgaattacttgaa

SEQ ID NO: 23 - RNA, 23 nt, Artificial Sequence
  Synthetic miR-18a-5p
uaaggugcaucuagugcagauag

SEQ ID NO: 24 - DNA, 23 bp, Artificial Sequence
  Synthetic miR-18a-5p binding site
ctatctgcactagatgcacctta

SEQ ID NO: 25 - RNA, 19 nt, Artificial Sequence
  Synthetic miR-1246
aauggauuuuuggagcagg

SEQ ID NO: 26 - DNA, 19 bp, Artificial Sequence
  Synthetic miR-1246 binding site
cctgctccaaaaatccatt

SEQ ID NO: 27 - RNA, 22 nt, Artificial Sequence
  Synthetic miR 126-3p
ucguaccgugaguaauaaugcg

SEQ ID NO: 28 - DNA, 22 bp, Artificial Sequence
  Synthetic miR 126-3p binding site
cgcattattactcacggtacga

SEQ ID NO: 29 - protein, 22 aa, Artificial Sequence
  Synthetic 2A peptide amino acid
GSGATNFSLLKQAGDVEENPGP

SEQ ID NO: 30 - DNA, 66 bp, Artificial Sequence
  Synthetic 2A peptide nucleotide
ggaagcggagctactaacttcagcctgctgaagcaggctggagacgtggaggagaaccctggacct

SEQ ID NO: 31 - DNA, 108 bp, Artificial Sequence
  Synthetic 2A peptide nucleotide
tccggactcagatccggggatctcaaaattgtcgctcctgtcaaacaaactcttaactttgatttactcaaactggctggggatgtagaaagcaatccaggtccactc

SEQ ID NO: 32 - RNA, 6 nt, Artificial Sequence
  Synthetic Kozak
gccrcc

SEQ ID NO: 33 - RNA, 10 nt, Artificial Sequence
  Synthetic V1
ccccggcgcc

SEQ ID NO: 34 - RNA, 7 nt, Artificial Sequence
  Synthetic V2
ccccggc

SEQ ID NO: 35 - RNA, 6 nt, Artificial Sequence
  Synthetic EK
gccgcc

SEQ ID NO: 36 - RNA, 41 nt, Artificial Sequence
  Synthetic 5′ UTR
gggaaauaagagagaaaagaagaguaagaagaaauauaaga

SEQ ID NO: 37 - RNA, 57 nt, Artificial Sequence
  Synthetic 5′ UTR
gggaaauaagagagaaaagaagaguaagaagaaauauaagaccccggcgccgccacc

SEQ ID NO: 38 - RNA, 47 nt, Artificial Sequence
  Synthetic Standard 5′ UTR
gggaaauaagagagaaaagaagaguaagaagaaauauaagagccacc

SEQ ID NO: 39 - RNA, 57 nt, Artificial Sequence; identical to SEQ ID NO: 37
  Synthetic V1-UTR

SEQ ID NO: 40 - RNA, 54 nt, Artificial Sequence
  Synthetic V2-UTR
gggaaauaagagagaaaagaagaguaagaagaaauauaagaccccggcgccacc

SEQ ID NO: 41 - RNA, 9 nt, Artificial Sequence
  Synthetic Kozak consensus
ccrccaugg

SEQ ID NO: 42 - RNA, 47 nt, Artificial Sequence; identical to SEQ ID NO: 38
  Synthetic 5′ UTR-001

SEQ ID NO: 43 - RNA, 47 nt, Artificial Sequence
  Synthetic 5′ UTR-002
gggagaucagagagaaaagaagaguaagaagaaauauaagagccacc

SEQ ID NO: 44 - RNA, 42 nt, Artificial Sequence
  Synthetic 5′ UTR-004
gggagacaagcuuggcauuccgguacuguugguaaagccacc

SEQ ID NO: 45 - RNA, 47 nt, Artificial Sequence; identical to SEQ ID NO: 43
  Synthetic 5′ UTR-005

SEQ ID NO: 46 - RNA, 42 nt, Artificial Sequence; identical to SEQ ID NO: 44
  Synthetic 5′ UTR-007

SEQ ID NO: 47 - RNA, 47 nt, Artificial Sequence
  Synthetic 5′ UTR-008
gggaauuaacagagaaaagaagaguaagaagaaauauaagagccacc

SEQ ID NO: 48 - RNA, 47 nt, Artificial Sequence
  Synthetic 5′ UTR-009
gggaaauuagacagaaaagaagaguaagaagaaauauaagagccacc

SEQ ID NO: 49 - RNA, 47 nt, Artificial Sequence
  Synthetic 5′ UTR-010
gggaaauaagagaguaaagaacaguaagaagaaauauaagagccacc

SEQ ID NO: 50 - RNA, 47 nt, Artificial Sequence
  Synthetic 5′ UTR-011
gggaaaaaagagagaaaagaagacuaagaagaaauauaagagccacc

SEQ ID NO: 51 - RNA, 47 nt, Artificial Sequence
  Synthetic 5′ UTR-012
gggaaauaagagagaaaagaagaguaagaagauauauaagagccacc

SEQ ID NO: 52 - RNA, 47 nt, Artificial Sequence
  Synthetic 5′ UTR-013
gggaaauaagagacaaaacaagaguaagaagaaauauaagagccacc

SEQ ID NO: 53 - RNA, 47 nt, Artificial Sequence
  Synthetic 5′ UTR-014
gggaaauuagagaguaaagaacaguaaguagaauuaaaagagccacc

SEQ ID NO: 54 - RNA, 47 nt, Artificial Sequence
  Synthetic 5′ UTR-015
gggaaauaagagagaauagaagaguaagaagaaauauaagagccacc

SEQ ID NO: 55 - RNA, 47 nt, Artificial Sequence
  Synthetic 5′ UTR-016
gggaaauaagagagaaaagaagaguaagaagaaaauuaagagccacc

SEQ ID NO: 56 - RNA, 47 nt, Artificial Sequence
  Synthetic 5′ UTR-017
gggaaauaagagagaaaagaagaguaagaagaaauuuaagagccacc

SEQ ID NO: 57 - RNA, 92 nt, Artificial Sequence
  Synthetic 5′ UTR-018
ucaagcuuuuggacccucguacagaagcuaauacgacucacuauagggaaauaagagagaaaagaagaguaagaagaaauauaagagccacc

SEQ ID NO: 58 - RNA, 142 nt, Artificial Sequence
  Synthetic 3′ UTR with miR-142-3p binding site
ugauaauaguccauaaaguaggaaacacuacagcuggagccucgguggccaugcuucuugccccuugggccuccccccagccccuccuccccuuccugcacccguacccccguggucuuugaauaaagucugagugggcggc

SEQ ID NO: 59 - RNA, 142 nt, Artificial Sequence
  Synthetic 3′ UTR with miR-142-3p binding site
ugauaauaggcuggagccucgguggcuccauaaaguaggaaacacuacacaugcuucuugccccuugggccuccccccagccccuccuccccuuccugcacccguacccccguggucuuugaauaaagucugagugggcggc

SEQ ID NO: 60 - RNA, 142 nt, Artificial Sequence
  Synthetic 3′ UTR with miR-142-3p binding site
ugauaauaggcuggagccucgguggccaugcuucuugccccuuccauaaaguaggaaacacuacaugggccuccccccagccccuccuccccuuccugcacccguacccccguggucuuugaauaaagucugagugggcggc

SEQ ID NO: 61 - RNA, 142 nt, Artificial Sequence
  Synthetic 3′ UTR with miR-142-3p binding site
ugauaauaggcuggagccucgguggccaugcuucuugccccuugggccuccccccaguccauaaaguaggaaacacuacaccccuccuccccuuccugcacccguacccccguggucuuugaauaaagucugagugggcggc

SEQ ID NO: 62 - RNA, 142 nt, Artificial Sequence
  Synthetic 3′ UTR with miR-142-3p binding site
ugauaauaggcuggagccucgguggccaugcuucuugccccuugggccuccccccagccccuccuccccuucuccauaaaguaggaaacacuacacugcacccguacccccguggucuuugaauaaagucugagugggcggc

SEQ ID NO: 63 - RNA, 142 nt, Artificial Sequence
  Synthetic 3′ UTR with miR-142-3p binding site
ugauaauaggcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 64 - RNA, 142 nt, Artificial Sequence
  Synthetic 3′ UTR with miR-142-3p binding site
ugauaauaggcuggagccucgguggccaugcuucuugccccuugggccuccccccagccccuccuccccuuccugcacccguacccccguggucuuugaauaaaguuccauaaaguaggaaacacuacacugagugggcggc

SEQ ID NO: 65 - RNA, 116 nt, Artificial Sequence
  Synthetic 3′ UTR-018
uaauaggcuggagccucgguggccaugcuucuugccccuugggccuccccccagccccuccuccccuuccugcacccguacccccguggucuuugaauaaagucugagugggcggc

SEQ ID NO: 66 - RNA, 164 nt, Artificial Sequence
  Synthetic 3′ UTR with miR-142-3p binding site variant 1
ugauaauaguccauaaaguaggaaacacuacagcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 67 - RNA, 164 nt, Artificial Sequence
  Synthetic 3′ UTR with miR-142-3p binding site variant 2
ugauaauaguccauaaaguaggaaacacuacagcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 68 - RNA, 142 nt, Artificial Sequence
  Synthetic 3′ UTR with miR-142-3p binding site variant 3
ugauaauaggcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 69 - DNA, 29 bp, Artificial Sequence
  Synthetic T7 promoter sequence
  misc_feature (21)..(29): n is a, c, g, or t
taatacgactcactatagggnnnnnnnnn

SEQ ID NO: 70 - DNA, 18 bp, Artificial Sequence
  Synthetic T7 promoter
taatacgactcactatag

SEQ ID NO: 71 - RNA, 23 nt, Artificial Sequence
  Synthetic miR-155-5p binding site
uuaaugcuaaucgugauaggggu

SEQ ID NO: 72 - DNA, 23 bp, Artificial Sequence
  Synthetic miR-155-5p binding site
acccctatcacgattagcattaa

SEQ ID NO: 73 - DNA, 92 bp, Artificial Sequence
  Synthetic 3′ UTR
tcaagcttttggaccctcgtacagaagctaatacgactcactatagggaaataagagagaaaagaagagtaagaagaaatataagagccacc

SEQ ID NO: 74 - DNA, 119 bp, Artificial Sequence
  Synthetic 3′ UTR
tgataataggctggagcctcggtggccatgcttcttgccccttgggcctccccccagcccctcctccccttcctgcacccgtacccccgtggtctttgaataaagtctgagtgggcggc

SEQ ID NO: 75 - DNA, 164 bp, Artificial Sequence
  Synthetic 3′ UTR with miR-122 and miR-142-3p sites
tgataataggctggagcctcggtggccatgcttcttgccccttgggcccaaacaccattgtcacactccatccccccagcccctcctccccttcctccataaagtaggaaacactacatgcacccgtacccccgtggtctttgaataaagtctgagtgggcggc

SEQ ID NO: 76 - RNA, 30 nt, Artificial Sequence
  Synthetic repeating GC-motif
  misc_feature (1)..(30): This region contains n repeats of “CCG” wherein n = 1 to 10, n= 2 to 8, n= 3 to 6, or n= 4 to 5; some positions may or may not be present
ccgccgccgccgccgccgccgccgccgccg

SEQ ID NO: 77 - RNA, 15 nt, Artificial Sequence
  Synthetic repeating GC-motif
  misc_feature (1)..(15): This region contains n repeats of “CCG” wherein n = 1, 2, 3, 4 or 5; some positions may or may not be present
ccgccgccgccgccg

SEQ ID NO: 78 - RNA, 12 nt, Artificial Sequence
  Synthetic repeating GC-motif
ccgccgccgccg

SEQ ID NO: 79 - RNA, 15 nt, Artificial Sequence
  Synthetic repeating GC-motif
ccgccgccgccgccg

SEQ ID NO: 80 - RNA, 30 nt, Artificial Sequence; identical to SEQ ID NO: 76
  Synthetic repeating GC-motif
  misc_feature (1)..(30): This region contains n repeats of “CCG” wherein n = 1 - 10; some positions may or may not be present

SEQ ID NO: 81 - RNA, 30 nt, Artificial Sequence
  Synthetic repeating GC-motif
  misc_feature (1)..(30): This region contains n repeats of “GCC” wherein n = 1 - 10; some positions may or may not be present
gccgccgccgccgccgccgccgccgccgcc

SEQ ID NO: 82 - DNA, 29 bp, Artificial Sequence
  Synthetic T7 promoter sequence
  misc_feature (20)..(29): n is a, c, g, or t
taatacgactcactataagnnnnnnnnnn

SEQ ID NO: 83 - DNA, 29 bp, Artificial Sequence
  Synthetic T7 promoter sequence
  misc_feature (1)..(10): n is a, c, g, or t
  misc_feature (20)..(29): n is a, c, g, or t
attatgctgagtgatattcnnnnnnnnnn

(19) United States
(12) Patent Application Publication
(10) Pub. No.: US 2023/0086537 A1
(43) Pub. Date: Mar. 23, 2023

(54) DIFFERENTIALLY EXPRESSED IMMUNE CELL MICRORNAS FOR REGULATION OF PROTEIN EXPRESSION

(71) Applicant: ModernaTX, Inc., Cambridge, MA (US)

(72) Inventors: Ruchi JAIN, Brookline, MA (US); Gilles BESIN, Medford, MA (US); Elizaveta ANDRIANOVA, Cambridge, MA (US)

(21) Appl. No.: 17/608,359

(22) PCT Filed: May 7, 2020
(86) PCT No.: PCT/US2020/031885

§ 371 Date: Nov. 2, 2021

Related U.S. Application Data
(60) Provisional application No. 62/844,704, filed on May 7, 2019.

(51) Int. Cl. C12N 15/88; A61K 31/7088

(52) U.S. Cl. CPC C12N 15/88; C12N 2330/10; A61K 31/7088

(57) ABSTRACT

The disclosure features mRNAs engineered with one or more microRNA binding sites targeted by a microRNA(s) that are differentially expressed in a target immune cell relative to a plurality of non-target immune cells. The disclosure also features methods of using the same, for example, for selectively degrading the mRNA in the target immune cell relative to the plurality of non-target immune cells.

### RELATED APPLICATION

This application claims the benefit of U.S. Provisional Application Ser. No. 62/844,704, filed on May 7, 2019, which is hereby incorporated by reference herein in its entirety.

### BACKGROUND

Biologics, such as recombinant antibodies, cytokines and growth factors, have been shown to be effective in the treatment of a wide variety of diseases and the FDA has now approved a large number of such agents for use in humans (for a review, see Kinch, M. S. (2015) Drug Discov. Today 20:393-398). The vast majority of FDA approved biologics are protein-based agents. More recently, messenger RNA-based agents are being developed as a disruptive therapeutic modality. In recent years, the development and use of mRNA as a therapeutic agent has demonstrated potential for treatment of numerous diseases and for the development of novel approaches in oncology, regenerative medicine and vaccination (Stanton et al (2017) RNA Therapeutics. Topics in Medicinal Chemistry, vol 27).

It is recognized that the control and regulation of mRNA translation is an important development component in order for this class of drugs to establish the desired therapeutic effect. Accordingly, new approaches and methods for use of mRNA-based agents in a subject, such as mRNA-based therapeutic agents, are needed, particularly methods that offer advantageous properties with regard to the safety and/or therapeutic efficacy of the mRNA-based agent in the subject. In particular, improvements that would allow selective expression or degredation of mRNA in immune cells would be of great benefit.

### SUMMARY OF DISCLOSURE

The present disclosure provides polynucleotides, including messenger RNAs (mRNAs), engineered with microRNA (miR)-binding sites to regulate expression of target proteins in select immune cell populations (e.g., target immune cell populations).

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target human immune cell relative to a plurality of non-target human immune cells, wherein the target human immune cell and the non-target immune cell are selected from (a) different immune cell types, (b) different immune cell states, and (c) different immune cell subpopulations, such that the mRNA is selectively degraded in the target human immune cell relative to the plurality of non-target human immune cells.

In some embodiments, the target immune cell and the plurality of non-target immune cell are of different immune cell types, optionally between 2 and 5 different immune cell types, optionally greater than 3 different cell types, optionally greater than 4 different cell types.

In some embodiments, the target immune cell and the plurality of non-target immune cell are of different immune cell states, optionally, wherein the plurality of non-target immune cells comprises between 2 and 5 different immune cell states, optionally wherein the plurality of non-target immune cells comprises greater than 3 different cell states, optionally wherein the plurality of non-target immune cells comprises greater than 4 different cell states.

In some embodiments, the target immune cell and the plurality of non-target immune cells are of different activation states. In some embodiments, the target immune cell and the plurality of non-target immune cells are of different transformation states. In some embodiments, the target immune cell and the plurality of non-target immune cells are of different immune cell subpopulations.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target human T cell relative to a plurality of non-target human immune cells comprising at least two cell types selected from the group consisting of: a dendritic cell (DC), a neutrophil, a natural killer (NK) cell, a monocyte, and a macrophage, wherein the target human immune cell and the non-target immune cell are selected from (a) different immune cell types, (b) different immune cell states, and (c) different immune cell subpopulations, such that the mRNA is selectively degraded in the target human immune cell relative to the plurality of non-target human immune cells. In some embodiments, the target immune cell is a human T cell and the miR is selected from the group consisting of: miR-146, miR-23a, miR-142, miR-150, miR-21, and a combination thereof.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target human DC relative to a plurality of non-target human immune cells comprising at least two cell types selected from the group consisting of: a T cell, a neutrophil, an NK cell, a monocyte, and a macrophage, wherein the target human immune cell and the non-target immune cell are selected from (a) different immune cell types, (b) different immune cell states, and (c) different immune cell subpopulations, such that the mRNA is selectively degraded in the target human immune cell relative to the plurality of non-target human immune cells. In some embodiments, the target immune cell is a DC and the miR is selected from the group consisting of: miR-142, miR-223, and a combination miR-142 and miR-223.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target human neutrophil relative to a plurality of non-target human immune cells comprising at least two cell types selected from the group consisting of: a T cell, DC, an NK cell, a monocyte, and a macrophage, wherein the target human immune cell and the non-target immune cell are selected from (a) different immune cell types, (b) different immune cell states, and (c) different immune cell subpopulations, such that the mRNA is selectively degraded in the target human immune cell relative to the plurality of non-target human immune cells. In some embodiments, the target immune cell is a neutrophil and the miR is selected from the group consisting of: miR-143, miR-23a, miR-142, miR-150, and a combination thereof.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target human NK cell relative to a plurality of non-target human immune cells comprising at least two cell types selected from the group consisting of: a T cell, DC, a neutrophil, a monocyte, and a macrophage, wherein the target human immune cell and the non-target immune cell are selected from (a) different immune cell types, (b) different immune cell states, and (c) different immune cell subpopulations, such that the mRNA is selectively degraded in the target human immune cell relative to the plurality of non-target human immune cells. In some embodiments, the target immune cell is an NK cell and the miR is selected from the group consisting of: miR-146, miR-23a, miR-142, miR-223, and a combination thereof.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target human monocyte relative to a plurality of non-target human immune cells comprising at least two cell types selected from the group consisting of: a T cell, DC, a neutrophil, an NK cell, and a macrophage, wherein the target human immune cell and the non-target immune cell are selected from (a) different immune cell types, (b) different immune cell states, and (c) different immune cell subpopulations, such that the mRNA is selectively degraded in the target human immune cell relative to the plurality of non-target human immune cells. In some embodiments, the target immune cell is a monocyte and the miR is selected from the group consisting of: miR-23a, miR-142, miR-223, and a combination thereof.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target human macrophage relative to a plurality of non-target human immune cells comprising at least two cell types selected from the group consisting of: a T cell, DC, a neutrophil, an NK cell, and a monocyte, wherein the target human immune cell and the non-target immune cell are selected from (a) different immune cell types, (b) different immune cell states, and (c) different immune cell subpopulations, such that the mRNA is selectively degraded in the target human immune cell relative to the plurality of non-target human immune cells. In some embodiments, the target immune cell is a macrophage and the miR is selected from the group consisting of: miR-23a, miR-142, miR-223, and a combination thereof.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in an activated T cell relative to an unstimulated T cell. In some embodiments, the miR expressed in an activated T cell is miR-155a-5p or miR-132-3p, or a combination thereof.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a normal immune cell relative to a cancerous immune cell. In some embodiments, the normal immune cell is a bone marrow cell, a B cell, a T cell, a monocyte, a macrophage, a dendritic cell or any combination thereof. In some embodiments, the cancerous immune cell is an AML cell. In some embodiments, the miR which is differentially expressed in a normal immune cell is selected from miR-150-5p, miR-146b-5p, miR-4286, miR-579-3b, miR-4516, miR-146a-5p, miR-664b-3p, miR-342-3p, miR-342-5p, miR-1915-3p, miR-26b-5p and a combination thereof.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a cancerous immune cell relative to a normal immune cell. In some embodiments, the normal immune cell is a bone marrow cell, a B cell, a T cell, a monocyte, a macrophage, a dendritic cell or any combination thereof. In some embodiments, the cancerous immune cell is an AML cell. In some embodiments, the miR which is differentially expressed in a cancerous immune cell (e.g., AML cell) is selected from miR-18a-5p, miR-1246, miR-126-3p, and a combination thereof.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed a regulatory T cell relative to naïve T cells and effector T cells. In some embodiments, the miR is miR-146a-5p.

In any of the foregoing or related embodiments, the miR is abundantly expressed in the target immune cell relative to the plurality of non-target immune cells.

In any of the foregoing or related embodiments, the polypeptide of interest is selected from a secreted protein, an intracellular protein, a transmembrane, a membrane-bound protein, and a cytotoxic polypeptide.

In some embodiments, the disclosure provides an mRNA comprising 2-5 miR-binding sites, two miR-binding sites, three miR-binding sites, or 4 miR-binding sites.

In any of the foregoing or related embodiments, the miR-binding site(s) is located in the 3′ UTR. In some embodiments, the miR-binding site(s) is located in the 5′ UTR.

In any of the foregoing or related embodiments, the mRNA is fully modified with chemically-modified uridines, optionally wherein the chemically-modified uridines comprise N1-methyl-pseudouridine (m1ψ), 5-methoxy-uridine (mo5U), 5-methyl-cytidine (m5C), pseudouridine (ψ), α-thio-guanosine, or α-thio-adenosine, or a combination thereof, optionally wherein the chemically-modified uridines are N1-methylpseudouridines (m1ψ).

In some embodiments, the disclosure provides a lipid nanoparticle (LNP) comprising the mRNA of the disclosure. In some embodiments, the LNP comprises:

(i) an ionizable lipid;

(ii) a sterol or other structural lipid;

(iii) an mRNA molecule of the disclosure;

(iii) optionally, a non-cationic helper lipid or phospholipid; and

(iv) optionally, a PEG-lipid,

optionally, wherein one or more of (i) the ionizable lipid or (ii) the sterol or other structural lipid comprises an immune cell delivery potentiating lipid in an amount effective to enhance delivery of the LNP to a target immune cell and a plurality of non-target immune cells.

In some embodiments, the disclosure provides a pharmaceutical composition comprising the mRNA or the LNP of the disclosure, and a pharmaceutically acceptable carrier.

In some embodiments, the disclosure provides use of the pharmaceutical composition of the disclosure in treating or delaying progression of a disease or disorder in a subject, wherein the treatment comprises administration of the pharmaceutical composition.

In some embodiments, the disclosure provides for the use of the pharmaceutical composition in the manufacture of a medicament for treating or delaying progression of a disease or disorder in a subject, wherein the medicament comprises the pharmaceutical composition, and wherein the treatment comprises administration of the medicament.

In some embodiments, the disclosure provides a kit comprising a container comprising the pharmaceutical composition of the disclosure, and a package insert comprising instructions for administration of the pharmaceutical composition for treating or delaying progression of a disease or disorder in a subject.

In some embodiments, the disclosure provides a method of inducing selective degradation of an mRNA in a target immune cell relative to a plurality of non-target immune cells, comprising contacting a target immune cell with an mRNA or an LNP of the disclosure, optionally with a pharmaceutically acceptable carrier, such that the mRNA is selectively degraded in the target immune cell relative to the plurality of non-target immune cells.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target immune cell relative to a plurality of non-target immune cells, such that the mRNA is selectively degraded in the target immune cell relative to the plurality of non-target immune cells.

In some embodiments, the target immune cell and the plurality of non-target immune cells are human immune cells. In some embodiments, the target immune cell and the plurality of non-target immune cell are of different immune cell types.

In some embodiments, the plurality of non-target immune cells comprises between 2 and 5 different immune cell types. In some embodiments, the plurality of non-target immune cells comprises greater than 3 different cell types. In some embodiments, the plurality of non-target immune cells comprises greater than 4 different cell types.

In some embodiments, the target immune cell and the plurality of non-target immune cell are of different immune cell states. In some embodiments, the plurality of non-target immune cells comprises between 2 and 5 different immune cell states. In some embodiments, the plurality of non-target immune cells comprises greater than 3 different cell states. In some embodiments, the plurality of non-target immune cells comprises greater than 4 different cell states.

In some embodiments, the target immune cell and the plurality of non-target immune cells are of different immune cell lineages. In some embodiments, the target immune cell and the plurality of non-target immune cells are of different immune cell subpopulations. In some embodiments, the target immune cell and the plurality of non-target immune cells are of different activation states. In some embodiments, the target immune cell and the plurality of non-target immune cells are of different transformation states.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target immune cell relative to a plurality of non-target immune cells, wherein the target immune cell is a T cell, such that the mRNA is selectively degraded in the target T cell relative to the plurality of non-target immune cells.

In some embodiments when the target immune cell is a T cell, the miR is selected from the group consisting of: miR-146, miR-23a, miR-142, miR-150, miR-21, and a combination thereof.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target immune cell relative to a plurality of non-target immune cells, wherein the target immune cell is a dendritic cell (DC), such that the mRNA is selectively degraded in the target DC relative to the plurality of non-target immune cells.

In some embodiments when the target immune cell is a DC, the miR is selected from the group consisting of: miR-142, miR-223, and a combination miR-142 and miR-223.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target immune cell relative to a plurality of non-target immune cells, wherein the target immune cell is a neutrophil, such that the mRNA is selectively degraded in the target neutrophil relative to the plurality of non-target immune cells.

In some embodiments when the target immune cell is a neutrophil, the miR is selected from the group consisting of: miR-143, miR-23a, miR-142, miR-150, and a combination thereof.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target immune cell relative to a plurality of non-target immune cells, wherein the target immune cell is a natural killer (NK) cell such that the mRNA is selectively degraded in the target NK cell relative to the plurality of non-target immune cells.

In some embodiments when the target immune cell is a NK cell, the miR is selected from the group consisting of: miR-146, miR-23a, miR-142, miR-223, and a combination thereof.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target immune cell relative to a plurality of non-target immune cells, wherein the target immune cell is a monocyte, such that the mRNA is selectively degraded in the target monocyte relative to the plurality of non-target immune cells.

In some embodiments when the target immune cell is a monocyte, the miR is selected from the group consisting of: miR-23a, miR-142, miR-223, and a combination thereof.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target immune cell relative to a plurality of non-target immune cells, wherein the target immune cell is a macrophage, such that the mRNA is selectively degraded in the target macrophage relative to the plurality of non-target immune cells.

In some embodiments when the target immune cell is a macrophage, the miR is selected from the group consisting of: miR-23a, miR-142, miR-223, and a combination thereof.

In any of the foregoing or related embodiments, the plurality of non-target immune cells comprises at least two cell types selected from the group consisting of: a T cell, a DC, a neutrophil, an NK cell, a monocyte, and a macrophage.

In any of the foregoing or related embodiments, the target immune cell is a regulatory T (Treg) cell and the miR is differentially expressed in regulatory T cells relative to naïve T cells and effector T cells. In some embodiments when the target immune cell is a Treg cell, the miR is miR-146a-5p.

In any of the foregoing or related embodiments, the target immune cell is an activated T cell and the miR is differentially expressed in the activated T cell relative to an unstimulated T cell. In some embodiments when the target immune cell is an activated T cell, the miR is miR-155a-5p or miR-132-3p.

In any of the foregoing or related embodiments, the target immune cell is a normal immune cell and the miR is differentially expressed in the normal immune cell relative to a cancerous immune cell. In some embodiments, the normal immune cell is a bone marrow cell, a B cell, a T cell, a monocyte, a macrophage, a dendritic cell or any combination thereof. In some embodiments, the cancerous immune cell is an AML cell. In some embodiments when the target immune cell is a normal immune cell, the miR is selected from the group consisting of: miR-150-5p, miR-146b-5p, miR-4286, miR-579-3b, miR-4516, miR-146a-5p, miR-664b-3p, miR-342-3p, miR-342-5p, miR-1915-3p, miR-26b-5p, and any combination thereof.

In any of the foregoing or related embodiments, the target immune cell is a cancerous immune cell and the miR is differentially expressed in the cancerous immune cell relative to a normal immune cell. In some embodiments, the normal immune cell is a bone marrow cell, a B cell, a T cell, a monocyte, a macrophage, a dendritic cell or any combination thereof. In some embodiments, the cancerous immune cell is an AML cell. In some embodiments when the target immune cell is an AML cell, the miR is selected from the group consisting of: miR-18a-5p, miR-1246, miR-126-3p, and any combination thereof.

In some embodiments, the disclosure provides an mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target immune cell relative to a plurality of non-target immune cells, and wherein the miR is abundantly expressed in the target immune cell relative to the plurality of non-target immune cells, such that the mRNA is selectively degraded in the target immune cell relative to the plurality of non-target immune cells.

In any of the foregoing or related embodiments, the mRNA comprises an ORF encoding a polypeptide of interest, wherein the polypeptide of interest is a secreted protein. In any of the foregoing or related embodiments, the mRNA comprises an ORF encoding a polypeptide of interest, wherein the polypeptide of interest is an intracellular protein. In any of the foregoing or related embodiments, the mRNA comprises an ORF encoding a polypeptide of interest, wherein the polypeptide of interest is a transmembrane or membrane-bound protein. In any of the foregoing or related embodiments, the mRNA comprises an ORF encoding a polypeptide of interest, wherein the polypeptide of interest is a cytotoxic polypeptide.

In any of the foregoing or related embodiments, the mRNA comprises 2-5 miR-binding sites. In any of the foregoing or related embodiments, the mRNA comprises two miR-binding sites, three miR-binding sites, or 4 miR-binding sites. In any of the foregoing or related embodiments, the mRNA comprises at least one-miR binding site located in the 3′ UTR. In any of the foregoing or related embodiments, the mRNA comprises at least one-miR binding site located in the 5′ UTR.

In any of the foregoing or related embodiments, the mRNA is fully modified with chemically-modified uridines. In some embodiments, the chemically-modified uridines comprise N1-methyl-pseudouridine (m1ψ), 5-methoxy-uridine (mo5U), 5-methyl-cytidine (m5C), pseudouridine (ψ), α-thio-guanosine, or α-thio-adenosine, or a combination thereof. In some embodiments, the chemically-modified uridines are N1-methylpseudouridines (m1ψ). In some embodiments, the chemically-modified uridines are 5-methoxy-uridine (mo5U).

In some embodiments, the disclosure provides lipid nanoparticles (LNPs) comprising the mRNA of the disclosure.

In some embodiments, the disclosure provides a pharmaceutical composition comprising the mRNA of the disclosure or an LNP, and a pharmaceutically acceptable carrier.

In some embodiments, the disclosure provides a pharmaceutical composition comprising the mRNA of the disclosure or an LNP, and a pharmaceutically acceptable carrier for use in treating or delaying progression of a disease or disorder in a subject, wherein the treatment comprises administration of the pharmaceutical composition.

In some embodiments, the disclosure provides use of the pharmaceutical composition comprising the mRNA of the disclosure or an LNP, and a pharmaceutically acceptable carrier in the manufacture of a medicament for treating or delaying progression of a disease or disorder in a subject, wherein the medicament comprises the pharmaceutical composition, and wherein the treatment comprises administration of the medicament.

In some embodiments, the disclosure provides kits comprising a container comprising an mRNA of the disclosure, an LNP of the disclosure or a pharmaceutical composition of the disclosure and a package insert comprising instructions for administration of the mRNA, LNP or pharmaceutical composition for treating or delaying progression of a disease or disorder in a subject.

In some embodiments, the disclosure provides methods comprising use of an mRNA of the disclosure, an LNP of the disclosure or a pharmaceutical composition of the disclosure for inducing selective degradation of an mRNA in a target immune cell relative to a plurality of non-target immune cells, comprising contacting a target immune cell with an mRNA, an LNP, optionally with a pharmaceutically acceptable carrier, such that the mRNA is selectively degraded in the target immune cell relative to the plurality of non-target immune cells.

In some embodiments, the disclosure provides an immune cell delivery LNP comprising:
(i) an ionizable lipid;
(ii) a sterol or other structural lipid;
(iii) an mRNA molecule of the disclosure;
(iii) optionally, a non-cationic helper lipid or phospholipid; and
(iv) optionally, a PEG-lipid;

wherein one or more of (i) the ionizable lipid or (ii) the sterol or other structural lipid comprises an immune cell delivery potentiating lipid in an amount effective to enhance delivery of the LNP to a target immune cell and a plurality of non-target immune cells.

In some embodiments, the immune cell delivery LNP comprises a phytosterol or a combination of a phytosterol and cholesterol.

In some embodiments, the immune cell delivery LNP comprises a phytosterol, wherein the phytosterol is selected from the group consisting of β-sitosterol, stigmasterol, β-sitostanol, campesterol, brassicasterol, and combinations thereof.

In some embodiments, the immune cell delivery LNP comprises a phytosterol, wherein the phytosterol comprises a sitosterol or a salt or an ester thereof.

In some embodiments, the immune cell delivery LNP comprises a phytosterol, wherein the phytosterol comprises a stigmasterol or a salt or an ester thereof.

In some embodiments, the immune cell delivery LNP comprises a phytosterol, wherein the phytosterol is beta-sitosterol

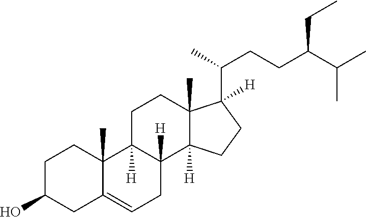

or a salt or an ester thereof.

In some embodiments, the immune cell delivery LNP comprises a phytosterol, wherein the phytosterol or a salt or ester thereof is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof.

In some embodiments, the immune cell delivery LNP comprises a phytosterol, wherein the phytosterol is β-sitosterol.

In some embodiments, the immune cell delivery LNP comprises a phytosterol, wherein the phytosterol is β-sitostanol.

In some embodiments, the immune cell delivery LNP comprises a phytosterol, wherein the phytosterol is campesterol.

In some embodiments, the immune cell delivery LNP comprises a phytosterol, wherein the phytosterol is brassicasterol.

In some embodiments, the immune cell delivery LNP comprises an ionizable lipid, wherein the ionizable lipid comprises a compound of any of Formulae (I I), (I IA), (I IB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8).

In some embodiments, the immune cell delivery LNP comprises an ionizable lipid, wherein the ionizable lipid comprises a compound selected from the group consisting of Compound X, Compound Y, Compound I-48, Compound I-50, Compound I-109, Compound I-111, Compound I-113, Compound I-181, Compound I-182, Compound I-244, Compound I-292, Compound I-301, Compound I-309, Compound I-317, Compound I-321, Compound I-322, Compound I-326, Compound I-328. Compound I-330, Compound I-331, Compound I-332, Compound I-347, Compound I-348, Compound I-349, Compound I-350, Compound I-352 and Compound I-M.

In some embodiments, the immune cell delivery LNP comprises an ionizable lipid, wherein the ionizable lipid comprises a compound selected from the group consisting of Compound X, Compound Y, Compound I-321, Compound I-292, Compound I-326, Compound 1-182, Compound I-301, Compound I-48, Compound I-50, Compound I-328, Compound I-330, Compound I-109, Compound I-111 and Compound I-181.

In some embodiments, the immune cell delivery LNP comprises a phospholipid, wherein the phospholipid comprises a compound selected from the group consisting of DSPC, DMPE, and Compound H-409.

In some embodiments, the immune cell delivery LNP comprises a PEG-lipid.

In some embodiments, the immune cell delivery LNP comprises a PEG-lipid, wherein the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the immune cell delivery LNP comprises a PEG lipid, wherein the PEG lipid comprises a compound selected from the group consisting of Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25.

In some embodiments, the immune cell delivery LNP comprises a PEG lipid, wherein the PEG lipid comprises a compound selected from the group consisting of Compound P-428, Compound PL-16, Compound PL-17, Compound PL-18, Compound PL-19, Compound PL-1, and Compound PL-2.

In some embodiments, the immune cell delivery LNP comprises about 30 mol % to about 60 mol % ionizable lipid, about 0 mol % to about 30 mol % non-cationic helper lipid or phospholipid, about 18.5 mol % to about 48.5 mol % sterol or other structural lipid, and about 0 mol % to about 10 mol % PEG lipid.

In some embodiments, the immune cell delivery LNP comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid.

In some embodiments, the immune cell delivery LNP comprises about 50 mol % ionizable lipid, about 10 mol % non-cationic helper lipid or phospholipid, about 38.5 mol % sterol or other structural lipid, and about 1.5 mol % PEG lipid.

In some embodiments, the immune cell delivery LNP comprises 18.5% phytosterol and the total mol % structural lipid is 38.5%.

In some embodiments, the immune cell delivery LNP comprises 28.5% phytosterol and the total mol % structural lipid is 38.5%.

In some embodiments, the immune cell delivery LNP comprises:

(i) about 50 mol % ionizable lipid, wherein the ionizable lipid is a compound selected from the group consisting of Compound I-301, Compound I-321, and Compound I-326;

(ii) about 10 mol % phospholipid, wherein the phospholipid is DSPC;

(iii) about 38.5 mol % structural lipid, wherein the structural lipid is selected from f3-sitosterol and cholesterol; and

(iv) about 1.5 mol % PEG lipid, wherein the PEG lipid is Compound P-428.

In any of the foregoing or related embodiments, the disclosure provides use of the immune cell delivery LNP of the disclosure, and an optional pharmaceutically acceptable carrier, in the manufacture of a medicament for inhibiting an immune response in an individual, wherein the medicament comprises the LNP and an optional pharmaceutically acceptable carrier and wherein the treatment comprises administration of the medicament, and an optional pharmaceutically acceptable carrier.

In other embodiments, the disclosure pertains to a method for inducing selective degradation of an mRNA in a target immune cell relative to a plurality of non-target immune cells in a subject, the method comprising administering to a subject in need thereof an immune cell delivery LNP of the disclosure, or pharmaceutical composition thereof.

In any of the foregoing or related aspects, the disclosure provides a method for treating a subject, for example a subject having a disease or condition that would benefit from inducing selective degradation of an mRNA in a target immune cell relative to a plurality of non-target immune cells in the subject. The treatment method comprises administering to a subject in need thereof any of the foregoing or related immune cell delivery LNPs. In some aspects, the immuno cell delivery LNP is administered in combination with another therapeutic agent.

### BRIEF DESCRIPTION OF THE FIGURES

FIGS. 1A-1F provide heat maps showing the thirty most differentially expressed miRs in B cells (FIG. 1A), T cells (FIG. 1B), monocytes (FIG. 1C), macrophages (FIG. 1D), dendritic cells (FIG. 1E), and bone marrow cells (FIG. 1F) shown as a relative abundance compared to other immune cell types.

FIGS. 2A-2F provide pie charts showing microRNA (miR) expression in healthy bone marrow (FIG. 2A), B cells (FIG. 2B), T cells (FIG. 2C), monocytes (FIG. 2D), macrophages (FIG. 2E), and dendritic cells (FIG. 2F), as a percentage of total detectable miRs.

FIG. 3 provides pie charts showing microRNA (miR) expression in mouse B cells (FIG. 3A), T cells (FIG. 3B), macrophages (FIG. 3C), monocytes (FIG. 3D), and dendritic cells (FIG. 3E) as a percentage total detectable miRs.

FIG. 4 provides contour plots showing expression of OX40L in human T cells and dendritic cells following transfection of the cells with LNP containing mRNA comprising an open reading frame (ORF) encoding OX40L and a 3′UTR comprising no miR binding site (miRLess), three miR-223-3p binding sites, or three miR-142-3p binding sites

FIG. 5 provides contour plots showing expression of OX40L in human T cells, B cells, dendritic cells, and monocytes following transfection of the cells with LNP containing mRNA comprising an ORF encoding OX40L and a 3′UTR comprising no miR binding site (miRLess), three miR-150-5p binding sites, three miR-21-5p binding sites, or three miR-23a-3p binding sites.

FIG. 6 provides contour plots showing expression of OX40L in human T cells, B cells, dendritic cells, and monocytes transfection of the cells with LNP containing mRNA comprising an ORF encoding OX40L and a 3′UTR comprising no miR binding site (miRLess), three miR-132-3p binding sites, three miR-146a-5p binding sites, or three miR-155-5p binding sites.

FIGS. 7A-7C provide dot plots showing mOX40L positive cells among T cells (FIG. 7A), B cells (FIG. 7B) and CD11b⁺ monocyte/macrophage and dendritic cells (FIG. 7C) in Sprague Dawley rats after intravenous administration with mOX40L encoded mRNAs with or without 3′UTR modifications comprising a miR-142-3p binding site or three miR-142-3p binding sites. Mock injected (PBS) mice were used as negative controls. The y-axis represents the percentage of mOX40L+ immune cells in each immune cell population.

FIGS. 8A-8B provide bar graphs showing expression of OX40L in mouse CD3+ T cells harvested from animals administered an mRNA that encodes OX40L and comprising a 3′UTR engineered with: no miR binding sites, one or three miR-21-5p binding sites, or one or three miR-23a-3p binding sites. Shown in FIG. 8A is the percentage of CD3+ T cells expressing OX40L and shown in FIG. 8B is the average surface expression of OX40L on CD3+ T cells.

FIGS. 9A-9B provide bar graphs showing expression of OX40L in mouse CD3+ T cells that are CD4+ harvested from animals administered an mRNA that encodes OX40L and comprising a 3′UTR engineered with: no miR binding sites, one or three miR-21-5p binding sites, or one or three miR-23a-3p binding sites. Shown in FIG. 9A is the percentage of CD4+ T cells expressing OX40L and shown in FIG. 9B is the average surface expression of OX40L on CD4+ T cells.

FIGS. 10A-10B provide bar graphs showing expression of OX40L in mouse CD3+ T cells that are CD8+ harvested from animals administered an mRNA that encodes OX40L and comprising a 3′UTR engineered with: no miR binding sites, one or three miR-21-5p binding sites, or one or three miR-23a-3p binding sites. Shown in FIG. 10A is the percentage of CD8+ T cells expressing OX40L and shown in FIG. 10B is the average surface expression of OX40L on CD8+ T cells.

FIGS. 11A-11B provide bar graphs showing expression of OX40L in mouse B cells harvested from animals administered an mRNA that encodes OX40L and comprising a 3′UTR engineered with: no miR binding sites, one or three miR-21-5p binding sites, or one or three miR-23a-3p binding sites. Shown in FIG. 11A is the percentage of B cells expressing OX40L and shown in FIG. 11B is the average surface expression of OX40L on B cells.

FIGS. 12A-12B provide bar graphs showing expression of OX40L in mouse macrophages harvested from animals administered an mRNA that encodes OX40L and comprising a 3′UTR engineered with: no miR binding sites, one or three miR-21-5p binding sites, or one or three miR-23a-3p binding sites. Shown in FIG. 12A is the percentage of macrophages expressing OX40L and shown in FIG. 12B is the average surface expression of OX40L on macrophages.

FIGS. 13A-13B provide bar graphs showing expression of OX40L in mouse dendritic cells harvested from animals administered an mRNA that encodes OX40L and comprising a 3′UTR engineered with: no miR binding sites, one or three miR-21-5p binding sites, or one or three miR-23a-3p binding sites. Shown in FIG. 13A is the percentage of dendritic cells expressing OX40L and shown in FIG. 13B is the average surface expression of OX40L on dendritic cells.

FIGS. 14A-14F provide pie charts showing miR expression in mouse tumor lines including MLL-AF9 (FIG. 14A), B16F10 (FIG. 14B), C1498 (FIG. 14C), H22 (FIG. 14D), MC38R (resistant to immune checkpoint inhibitors) (FIG. 14E), and MC38S (sensitive to immune checkpoint inhibitors) (FIG. 14F), wherein the most abundant miRs are depicted as a percentage of total detectable miRs.

FIGS. 15A-15O provide pie charts showing miR expression in AML cell lines including KG-1 (FIG. 15A), THP1 (FIG. 15B), OCI-AML12 (FIG. 15C), Kasumil (FIG. 15D), EO1-1 (FIG. 15E), HL60 (FIG. 15F), HEL (FIG. 15G), K562 (FIG. 15H), molm13 (FIG. 15I), molm16 (FIG. 15J), mv411 (FIG. 15K), nomo1 (FIG. 15L), OCI-AML3 (FIG. 15M), OCI-AML5 (FIG. 15N), and Kasumi3 (FIG. 15O), wherein the most abundant miRs are depicted as a percentage of total detectable miRs.

FIGS. 16A-16B provide pie charts showing miR expression in AML cells derived from two different human donors, wherein the most abundant miRs are depicted as a percentage of total detectable miRs.

FIGS. 17A-17B are bar graphs showing differential miR expressions between healthy immune cells and AML cells. FIG. 17A represents miRs that have 10-fold higher expression levels in healthy cells than in AML cells, and FIG. 17B represents miRs that have 10-fold higher expression levels in AML cells compared to healthy cells.

FIG. 18 is a bar graph comparing miR-150 levels in AML patient samples, AML cell lines, and healthy donor immune cells.

FIGS. 19A-19B provide bar graphs showing GFP fluorescence intensity in HEL AML cell lines that have been transfected with eGFP encoded mRNAs having 3′UTR comprising: no miR binding sites, one or three miR-150 binding sites, one or three miR-142 binding sites; or mock transfected. In FIG. 19A, HEL cells were transfected with either 50 ng or 200 ng GFP mRNA encapsulated in lipid nanoparticles. Mean fluorescence intensity after 24 h was measured by flow cytometry is plotted on the y-axis. In FIG. 19B, HEL cells were transfected using 200 ng GFP mRNA and fluorescence was evaluated using Incucyte for 48 h. Y-axis represents area under the curve for green fluorescence intensity for this duration.

FIG. 20 provides a bar graph showing GFP fluorescence intensity in THP-1 AML cells that have been transfected using lipofectamine with 100 ng eGFP encoding mRNAs having 3′UTRs engineered with: no miR binding sites, one or three miR-150 binding sites, one or three miR-142 binding sites; or mock transfected. GFP fluorescence was evaluated using Incucyte for 48 h. Y-axis represents area under the curve for fluorescent intensity for this duration.

FIGS. 21A-21C provide pie charts showing miR expression in human naïve T cells (FIG. 21A), effector T cells (FIG. 21B), and regulatory T cells (FIG. 21C), as a percentage total detectable miRs.

FIGS. 22A-22B provide bar graphs showing differential expression of miRs between Treg and naïve T cell populations (FIG. 22A), and between Treg and effector T cell populations (FIG. 22B).

FIG. 23 provides the flow cytometric gating strategy for isolating T cell populations from PBMC cells. The isolated cell populations are then transfected with mOX40L, activated with a PMA/ionomycin cocktail, or mock transfected.

FIGS. 24A-24C provide pie charts showing miR expression in untreated CD3+ T cells (FIG. 24A), mOX40L transfected CD3+ T cells (FIG. 24B), and PMA/ionomycin treated CD3+ T cells (FIG. 24C).

FIG. 25 provides a bar graph showing differential expression of miRs in PMA/ionomycin treated vs. untreated CD3+ cells.

FIGS. 26A-26C provide pie charts showing miR expression in untreated CD3+CD4+ T cells (FIG. 26A), mOX40L transfected CD3+CD4+ T cells (FIG. 26B), and PMA/ionomycin treated CD3+CD4+ T cells (FIG. 26C).

FIG. 27 provides a bar graph showing differential expression of miRs in PMA/ionomycin treated vs. untreated CD3+CD4+ cells.

FIGS. 28A-28C provide pie charts showing relative miR levels in untreated CD3+CD8+ T cells (FIG. 28A), mOX40L transfected CD3+CD8+ T cells (FIG. 28B), and PMA/ionomycin treated CD3+CD8+ T cells (FIG. 28C).

FIG. 29 provides a bar graph showing differential expression of miRs in PMA/ionomycin treated vs. untreated CD3+CD8+ cells.

FIGS. 30A-30B provide bar graphs showing the percentage of cells positive for activation markers CD44+CD69+CD4+(FIG. 30A) and CD44+CD69+CD8+(FIG. 30B) in PBMCs after 24-, 48-, and 72-hours treatment with (i) anti-CD3 and anti-CD28, (ii) anti-CD3, anti-CD28, and anti-CD2; or (iii) PMA/Ionomycin. Unstimulated or PBS treated PBMCs were used as negative controls.

FIGS. 31A-31C provide dot plots showing miR-155 expression levels in two donor PBMCs population after 24-, 48-, and 72-hours treatment with (i) anti-CD3 and anti-CD28, (ii) anti-CD3, anti-CD28, and anti-CD2; or (iii) PMA/Ionomycin (“PI”).

FIG. 32 provides box plots showing normalized miR-155 expression levels in five donor PBMCs population after 24-hours treatment with (i) anti-CD3 and anti-CD28. (ii) anti-CD3, anti-CD28, and anti-CD2; or (iii) PMA/Ionomycin (“PI”).

FIGS. 33A-33B provide bar graphs showing mOX40L expression levels in T cells after transfection with mOX40L encoding mRNA constructs having zero, one, or three miR 155-binding site(s) in the 3′UTR. T cells were either untreated (resting T cells) or treated overnight with anti-CD3 and anti-CD28, or PMA/ionomycin. The T cells were then transfected witn LNP encapsulated mRNA and mOX40L expression was measured in a FACS assay. FIG. 33B shows the expression in resting T cells as shown in FIG. 33A but with a different y-axis scale.

### DETAILED DESCRIPTION

The present disclosure provides therapeutic mRNAs engineered with microRNA (miR)-binding sites to regulate expression of target proteins in select immune cell populations (e.g., target immune cell populations). The present disclosure is based, at least in part, on the identification of naturally-occurring miRs differentially expressed in select types of human immune cells (e.g., T cell, dendritic cell (DC), NK cells) relative to other human immune cell populations, or select immune cell states (e.g., activated vs. resting, normal vs. transformed). MiR-binding sites targeted by the differentially expressed miRs are exploited as translational switches to regulate mRNA expression in target immune cell populations relative to one or more non-target immune cell populations. Accordingly, the present disclosure provides mRNAs comprising at least one miR-binding site for a miR differentially expressed in a target immune cell population and methods for using the same to selectively regulate mRNA expression. The present mRNA compositions and methods allow for differential expression of a polypeptide of interest encoded by an mRNA in immune cell populations.

The present disclosure provides mRNAs comprising one or more miR-binding sites targeted by miRs that are differentially expressed in certain types of immune cell lineages. In some embodiments, the mRNAs of the disclosure comprise one or more miR-binding sites targeted by miRs that are differentially expressed in a target immune cell relative to a plurality of non-target immune cells (e.g., two, three, four, five or more types of non-target immune cells). In some embodiments, the present disclosure provides mRNAs comprising one or more miR-binding sites targeted by miRs that are differentially expressed in T cells, relative to a plurality of non-target immune cell types (e.g., bone marrow cells, B cells, monocytes, macrophages, and DCs). In some embodiments, miRs identified as being differentially expressed in T cells also have high expression in T cells (e.g., abundantly expressed). Exemplary miRs identified as being abundantly and differentially expressed in T cells include miR-194-5p, miR-92a-3p, miR-1260b, miR-542-5p and miR-190b. Accordingly, the present disclosure provides mRNAs comprising one or more miR-binding sites targeted by a miR, or a combination of two or more miR-binding sites targeted by different miRs, wherein each miR is differentially and, in some embodiments also abundantly, expressed in T cells relative to a plurality of other immune cell populations. In some embodiments, the miR-binding site is targeted by a miR selected from miR-194-5p, miR-92a-3p, miR-1260b, miR-542-5p and miR-190b, and any combination thereof.

In other embodiments, the present disclosure provides mRNAs comprising one or more miR-binding sites targeted by miRs that are differentially expressed in DCs relative to a plurality of non-target immune cell types (e.g., bone marrow cells, T cells, B cells, monocytes, and macrophages). In some embodiments, miRs identified as being differentially expressed in DCs also have high expression in DCs (e.g., abundantly expressed). Exemplary miRs identified as being abundantly and differentially expressed in DCs include miR-223-3p, 21-5p, 23a-3p, let-7d-3p, miR-191-5p. Accordingly, the present disclosure provides mRNAs comprising one or more miR-binding sites targeted by a miR, or a combination of two or more miR-binding sites targeted by different miRs, wherein each miR is differentially and, in some embodiments also abundantly, expressed in DCs relative to a plurality of other immune cell populations. In some embodiments, the miR-binding site is targeted by a miR selected from miR-223-3p. 21-5p, 23a-3p, let-7d-3p, miR-191-5p, and any combination thereof.

In other embodiments, the present disclosure provides mRNAs comprising one or more miR-binding sites targeted by a miR that are differentially expressed in monocytes compared to a plurality of non-target immune cell types (e.g., bone marrow cells, T cells, B cells, macrophages and DCs). In some embodiments, miRs identified as being differentially expressed in monocytes also have high expression in monocytes (e.g., abundantly expressed). Exemplary miRs identified as being abundantly and differentially expressed in monocytes include miR-4454, miR-7975, miR-181a-5p, miR-548aa, and miR-548t-3p. Accordingly, the present disclosure provides mRNAs comprising one or more miR-binding sites targeted by a miR, or a combination of two or more miR-binding sites targeted by different miRs, wherein each miR is differentially and, in some embodiments also abundantly, expressed in monocytes relative to a plurality of other immune cell populations. In some embodiments, the miR-binding site is targeted by a miR selected from miR-4454, miR-7975, miR-181a-5p, miR-548aa, and miR-548t-3p, and any combination thereof.

In other embodiments, the present disclosure provides mRNAs comprising one or more miR-binding sites targeted by a miR that is differentially expressed in macrophages compared to a plurality of non-target immune cell types (e.g., bone marrow cells, T cells, B cells, monocytes, and DCs). In some embodiments, miRs identified as being differentially expressed in macrophages also have high expression in macrophages (e.g., abundantly expressed). Exemplary miRs identified as being abundantly and differentially expressed in macrophages include miR-33b-5p, miR-346, miR-1205, miR-548a1, and miR-1228-3p. Accordingly, the present disclosure provides mRNAs comprising one or more miR-binding sites targeted by a miR, or a combination of two or more miR-binding sites targeted by different miRs, wherein each miR is differentially, in some embodiments also and abundantly, expressed in macrophages relative to a plurality of other immune cell populations. In some embodiments, the miR-binding site is targeted by a miR selected from miR-33b-5p, miR-346, miR-1205, miR-548al, and miR-1228-3p, and any combination thereof.

In other embodiments, the present disclosure provides mRNAs comprising one or more miR-binding sites targeted by a miR that is differentially expressed in B cells compared to a plurality of non-target immune cell types (e.g., bone marrow cells, T cells, monocytes, macrophages, and DCs). In some embodiments, miRs identified as being differentially expressed in B cells also have high expression in B cells (e.g., abundantly expressed). Exemplary miRs identified as being abundantly and differentially expressed in B cells include miR-223-3p, miR-1972, miR-548ah-5p, miR-1276, and miR-4531. Accordingly, the present disclosure provides mRNAs comprising one or more miR-binding sites targeted by a miR, or a combination of two or more miR-binding sites targeted by different miRs, wherein each miR is differentially and, in some embodiments also abundantly, expressed in B cells relative to a plurality of other immune cell populations. In some embodiments, the miR-binding site is targeted by a miR selected from miR-223-3p, miR-1972, miR-548ah-5p, miR-1276, and miR-4531, and any combination thereof.

In other embodiments, the present disclosure provides mRNAs comprising one or more miR-binding sites targeted by a miR that is differentially expressed in bone marrow cells compared to a plurality of noon-target immune cell types (e.g., T cells, B cells, monocytes, macrophages and DCs). In some embodiments, miRs identified as being differentially expressed in bone marrow cells also have high expression in bone marrow cells (e.g., abundantly expressed). Exemplary miRs identified as being abundantly and differentially expressed in bone marrow cells include miR-150-5p, miR-223-3p, miR-374a-5p, miR-16-6p, and let-7g-5p. Accordingly, the present disclosure provides mRNAs comprising one or more miR-binding sites targeted by a miR, or a combination of two or more miR-binding sites targeted by different miRs, wherein each miR is differentially and, in some embodiments also abundantly, expressed in bone marrow cells relative to a plurality of other immune cell populations. In some embodiments, the miR-binding site is targeted by a miR selected from miR-150-5p, miR-223-3p, miR-374a-5p, miR-16-6p, and let-7g-5p, and any combination thereof.

In some embodiments, the disclosure provides mRNAs comprising one or more miR-binding sites targeted by miRs that are identified as being highly or abundantly expressed in at least one type of target immune cells. In some embodiments, the present disclosure provides mRNAs comprising one or more miR-binding sites targeted by miRs that are identified as being highly or abundantly expressed in DCs. These include miR-223-3p, miR-21-5p and let-7a-5p. Accordingly, the present disclosure provides mRNAs comprising one or more miR-binding sites targeted by a miR, or a combination of two or more miR-binding sites targeted by different miRs, wherein each miR is highly or abundantly expressed in DCs, wherein the miR is selected from miR-223-3p, miR-21-5p and let-7a-5p, and any combination thereof. In some embodiments, the disclosure provides mRNAs comprising one or more miR-binding sites targeted by miRs that are identified as being highly or abundantly expressed in T cells. These include miR-342-3p. Accordingly, the present disclosure provides mRNAs comprising one or more miR-binding sites targeted by a miR which is highly or abundantly expressed T cells, wherein the miR is miR-342-3p.

In some embodiments, the disclosure provides mRNAs comprising one or more miR-binding sites targeted by miRs that are identified as being highly or abundantly expressed in at least two types of target immune cells. In some embodiments, the disclosure provides mRNAs comprising one or more miR-binding sites targeted by miRs identified as being highly or abundantly expressed in B cells and T cells. These include let-7g-5p. Accordingly, the present disclosure provides mRNAs comprising one or more miR-binding sites targeted by a miR which is highly or abundantly expressed in B cells and T cells, wherein the miR let-7g-5p. In other embodiments, the disclosure provides mRNAs comprising one or more miR-binding sites targeted by miRs identified as being highly or abundantly expressed in monocytes and macrophages. These include miR-4454 and miR-7975. Accordingly, the present disclosure provides mRNAs comprising one or more miR-binding sites targeted by a miR, or a combination of two or more miR-binding sites targeted by different miRs, wherein each miR is highly or abundantly expressed in monocytes and macrophages, wherein the miR is selected from miR-4454, miR-7975, and any combination thereof.

In some embodiments, the disclosure provides mRNAs comprising one or more miR-binding sites targeted by miRs that are identified as being highly or abundantly expressed in at least four types of target immune cells. In some embodiments, the disclosure provides mRNAs comprising one or more miR-binding sites targeted by a miR identified as being highly or abundantly expressed in B cells, T cells, monocytes, or macrophages. These include miR-150-5p and miR-29b-3p. Accordingly, the present disclosure provides mRNAs comprising one or more miR-binding sites targeted by a miR, or a combination of two or more miR-binding sites targeted by different miRs, wherein each miR is highly or abundantly expressed in B cells, T cells, monocytes and macrophages, wherein the miR is selected from miR-150-5p, miR-29b-3p, and any combination thereof. In other embodiments, the disclosure provides mRNAs comprising one or more miR-binding sites targeted by miRs identified as being highly or abundantly expressed in B cells, monocytes, macrophages, and DCs. These include miR-16a-5p. Accordingly, the present disclosure provides mRNAs comprising one or more miR-binding sites targeted by a miR that is highly or abundantly expressed in B cells, monocytes, macrophages, and DCs, wherein the miR is miR-16a-5p.

In some embodiments, the disclosure provides mRNAs comprising one or more miR-binding sites targeted by miRs identified as being highly or abundantly expressed at least five types of immune cells. In some embodiments, the disclosure provides mRNAs comprising one or more miR-binding sites targeted by miRs identified as being highly or abundantly expressed in B cells, T cells, monocytes, macrophages and DCs. These include miR-142-3p. Accordingly, the present disclosure provides mRNAs comprising one or more miR-binding sites targeted by a miR that is highly or abundantly expressed in B cells, T cells, monocytes, macrophages, and DCs, wherein the miR is miR-142-3p.

In other embodiments, the disclosure provides mRNAs comprising one or more miR-binding sites, wherein the mRNA has reduced expression in at least one type of target immune cell compared to a plurality of non-target immune cells, e.g., one, two, three, four, five or more non-target immune cell populations. In some embodiments, the disclosure provides mRNAs comprising one or more miR-binding sites targeted by miR-21-5p which have reduced expression in T cells, but not in DCs, monocytes, macrophages, neutrophils or NK cells. In other embodiments, the disclosure provides mRNAs comprising one or more miR-binding sites targeted by miR-143 which have reduced expression in neutrophils, but not in T cells, DCs, monocytes, macrophages, or NK cells.

In other embodiments, the disclosure provides mRNAs comprising one or more miR-binding sites, wherein the mRNA has reduced expression in at least one type of target immune cells compared to a plurality of up to four types of non-target immune cells. In some embodiments, the disclosure provides mRNAs comprising one or more miR-binding sites targeted by miR-146a-5p which have reduced expression in T cells and NK cells, but not in DCs, monocytes, macrophages or neutrophils. In other embodiments, the disclosure provides mRNAs comprising one or more miR-binding sites targeted by miR-150-5p which have reduced expression in T cells and neutrophils, but not in monocytes, macrophages, and DCs.

In other embodiments, the disclosure provides mRNAs comprising one or more miR-binding sites, wherein the mRNA has reduced expression in at least one type of target immune cells compared to a plurality of up to two types of non-target immune cells. In some embodiments, the disclosure provides mRNAs comprising one or more miR-binding sites targeted by miR-223-3p which have reduced expression in DCs, monocytes, macrophages, and NK cells, but not in T cells or neutrophils.

In other embodiments, the disclosure provides mRNAs comprising one or more miR-binding sites, wherein the mRNA has reduced expression in one or more type of target immune cells compared to at least one other type of non-target immune cells. In some embodiments, the disclosure provides mRNAs comprising one or more miR-binding sites targeted by miR-23a-3p which have reduced expression in T cells, monocytes, macrophages, neutrophils and NK cells, but not in DCs.

In other embodiments, the disclosure provides compositions and methods for engineering mRNAs comprising miR-binding sites to multiple miRs expressed in one or more target immune cells. In one embodiment, multiple miR-binding sites may be added to further reduce expression of a target protein in an immune cell. For example, a plurality of miR-binding sites that all promote enhanced degredation of mRNA within a particular cell type may be incorporated into an mRNA construct. In another embodiment, multiple miR binding sites may be included to reduce expression of a target protein in different types of immune cells. Without being bound by theory, it is believed that mRNAs comprising a combination of miR-binding sites of different immune cell specificity improves the selectivity of expression of an mRNA in one type of immune cell compared to a plurality of other types of immune cells. In one embodiment, the disclosure provides an mRNA comprising one or more binding sites targeted by miR-223-3p and one or more binding sites targeted by miR-143 to reduce expression of the mRNA in DCs, monocytes, macrophages, neutrophils and NK cells, but not in T cells. Thus, a combination of miR-binding sites targeted by different miRs with different immune cell specificity results in reduced expression of an mRNA across a plurality of immune cell types but not in a target immune cell type.

The present disclosure also provides mRNAs comprising one or more miR-binding sites targeted by a miR that is differentially expressed in immune cells of different transformation states (e.g., a cancerous cell vs. a healthy or non-cancerous cell). In some embodiments, the present disclosure provides mRNAs comprising one or more miR-binding sites targeted by miR-150p-5p which is highly expressed in normal immune cells (e.g., bone marrow cells, B cells, T, cells, monocytes and macrophages) compared to cancerous (e.g., AML) cells. Inclusion of miR-150-5p binding sites in a mRNA molecule suppresses expression of a polypeptide of interest (e.g., a cytotoxic polypeptide) in healthy immune cells, while maintaining the polypeptide expression in AML cells. Other miRs that are differentially xpressed in normal immune cells include, miR-146b-5p, miR-4286, miR-579-3p, miR-4516, miR-146a-5p, miR-664b-3p, miR-342-3p, miR-342-5p, miR-1915-3p, and miR-26b-5p. Accordingly, the present disclosure provides mRNAs comprising one or more miR-binding sites targeted by a miR that is differentially expressed in normal immune cells but not transformed cells (e.g., cancerous cells), wherein the miR is selected from miR-146b-5p, miR-4286, miR-579-3p, miR-4516, miR-146a-5p, miR-664b-3p, miR-342-3p, miR-342-5p, miR-1915-3p, and miR-26b-5p, and any combination thereof. The present disclosure also provides mRNAs comprising one or more miR-binding sites targeted by a miR that is differentially expressed in transformed immune cells (e.g., cancerous cells, e.g., AML cells) relative to normal immune cells. Accordingly, the present disclosure provides mRNAs comprising one or more miR-binding sites targeted by a miR that is differentially expressed in transformed immune cells but not normal immune cells, wherein the miR is selected from miR-126-3p, miR-1246, miR-18a-5p, and any combination thereof.

In other embodiments, the disclosure provides mRNAs comprising one or more miR-binding sites targeted by a miR that is differentially expressed in one immune cell state but not in another immune cell state (e.g., activated immune vs. unstimulated immune cell). Accordingly, the present disclosure provides mRNAs comprising one or more miR-binding sites targeted by a miR that is differentially expressed in activated T cells but not unstimulated T cells, wherein the miR is selected from miR-155-5p and miR-132-3p.

In other embodiments, the present disclosure provides mRNAs comprising one or more miR-binding sites targeted by a miR that is differentially expressed one immune cell type relative to another immune cell type within the same immune cell class (e.g., T regulatory vs. T effector or T naïve cells). Accordingly, the present disclosure provides mRNAs comprising one or more miR-binding sites targeted by a miR that is differentially expressed in regulatory T cells relative to naïve or effector T cells, such as miR-146a-5p.

### MicroRNAs

In some embodiments, a microRNA (miR) of the disclosure is expressed by specific tissue or cell type. Identification of naturally-occurring miRs and their differential expression pattern in certain tissue and cell types is described herein.

miRs are 19-25 nucleotides long non-coding RNAs that bind to an mRNA and down-regulate gene expression either by reducing mRNA stability or by inhibiting translation of the mRNA. miRs regulate expression of mRNA by functioning as sequence guides for recruiting a ribonucleoprotein (RNP) complex to an mRNA comprising a complimentary sequence. The RNP complex recruited by miR binding is the RNA-induced silencing complex (RISC). Binding of RISC to an mRNA results in dampened or reduced expression of the mRNA (Baek, D. et al. (2008) Nature 455:64-71; Selbach, M. et al. (2008) Nature 455:58-63). In some embodiments, mRNAs of the disclosure are engineered with one or more miR binding sites. Incorporation of at least one miR binding site that binds a miR expressed by a target cell provides a method of regulating mRNA translation in the target cell, wherein translation is reduced in a target cell that expresses the corresponding miR. In some embodiments, incorporation of at least one miR binding site for a miR that is highly expressed in a target cell but has reduced or no expression in one or more non-target cells provides a method for regulating mRNA translation in a cell type specific manner, wherein translation of the mRNA is reduced in the target cell but unaffected in the one or more non-target cells.

The majority of human miRs are encoded within intergenic regions of the chromosome or within intronic regions of genes, with the small remainder being encoded within exonic regions of genes (Hsu, P. et al (2006) Nucleic Acids Res. 34:D135-D139). miRs are predominantly transcribed by RNA Polymerase II, but can also be transcribed by RNA Polymerase III. miRs located within a gene are transcribed together with the host gene, while those located within intergenic regions comprise a promoter than enables transcription. In some embodiments, a miR of the disclosure is encoded within an intergenic region of the chromosome. In some embodiments, a miR encoded in an intergenic region comprises an upstream promoter that enables transcription. In some embodiments, a miR of the disclosure is encoded within an intronic region of a gene. In some embodiments, a miR of the disclosure is encoded within an exonic region of a gene. In some embodiments, a miR of the disclosure is transcribed by RNA Polymerase II. In some embodiments, a miR of the disclosure is transcribed by RNA Polymerase III.

The structure of primary miR transcripts is unique from other long non-coding RNAs and comprises a hairpin loop structure flanked by segments of single-stranded RNA. The miRNA primary transcript is processed within the nucleus by a microprocessing complex comprising the RNA-binding protein DGCR8 and RNase III Drosha to generate a 60 nucleotide pre-miR (Lee, et al (2003) Nature 425:415-419). The pre-miR is exported from the nucleus by Exportin-5 (Lund, et al (2003) Science 303:95-98). Within the cytoplasm the hairpin loop of the pre-miR is processed by RNase III Dicer to generate a mature miR that comprises one arm of the hairpin stem loop (e.g., the 5′ or 3′ arm of the stem loop). The mature miR is assembled with the RISC complex, wherein it functions as a guide to direct RISC-mediated silencing of mRNA comprising a sequence recognized by the miR guide.

Expression of miRs is differentially regulated depending upon the cell type, tissue type, disease state, or development state (Ambros (2004) Nature 431:350-355; Bartel (2004) Cell 116:281-297). Multiple mechanisms contribute to regulation of miR expression. In some embodiments, regulation of miR expression occurs at the transcriptional level. Non-limiting examples of mechanisms that regulate miR expression at the transcriptional level include changes in methylation of regulatory element in a miR-encoding genes or altered activity of a transcription factor responsible for transcription of a miR-encoding gene (Gulyaeva et al (2016) J Transl Med 14:143). In some embodiments, regulation of miR expression occurs at the post-transcriptional level. Non-limiting examples of mechanisms that regulate miR expression at the post-transcriptional level include changes in miR processing, changes in nuclear export of a miR, or changes in miR stability.

Identification of miRs and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/1eu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety). Analysis of miR expression levels can be determined according to any art-recognized method for measuring nucleic acid expression. For the purposes of analyzing the expression levels of multiple nucleic acids, for example miRs, across multiple cell types (across a plurality of cell types), any one of a variety of microarray or other multiplexed analysis methods are available to the skilled artisan, for example, digital multiplexed gene expression analysis using NanoString methodology (Curr Protoc Mol Biol. 2011 April; Chapter 25:Unit25B).

### Immune Cell MicroRNAs

In some embodiments, a miR of the disclosure is differentially expressed in one immune cell as compared to a plurality of other immune cells. The immune cells can be, e.g., of different types, different transformation states, different activation states, or different subsets.

In some embodiments, a miR is differentially expressed in a first type of immune cell relative to one additional type of immune cells. In some embodiments, a miR is differentially expressed in a first type of immune cell relative to at least two additional types of immune cells. In some embodiments, a miR is differentially expressed in a first type of immune cell relative to at least three additional types of immune cells. In some embodiments, a miR is differentially expressed in a first type of immune cell relative to at least four additional types of immune cells. In some embodiments, a miR is differentially expressed in a first type of immune cell relative to at least five additional types of immune cells. In some embodiments, a miR is differentially expressed in a first type of immune cell relative to at least six additional types of immune cells. In some embodiments, a miR is differentially expressed in a first type of immune cell relative to a second type of immune cells, a third type of immune cells, a fourth type of immune cells, a fifth type of immune cells and a sixth type of immune cells.

In some embodiments, a miR of the disclosure is differentially expressed by a type of immune cells (e.g., B cells, T cells, DCs, monocytes, macrophages or bone marrow cells). To determine or identify differentially expressed miRs, expression levels can be depicted in a heatmap, i.e., a graphical representation of data using a system of color-coding (or other such coding of intensity levels) to represent different values. A heat map (or heatmap) is a graphical representation of data where the individual values contained in a matrix are represented as colors or intensity. Nucleic acid heat maps are typically used in molecular biology to represent the level of expression of many nucleic acids across a number of comparable samples (e.g. cells in different states, different cell types, etc.) as they are obtained from nucleic acid, e.g., RNA, mRNA, or miR microarrays.

To determine differentially expressed miRs, i.e., miRs which are expressed at a higher level in a given immune cell type or state relative to a plurality of immune cell types or states, for example, in at least 3, at least 4, at least 5, or at least 6 different immune cell types, one begins by determining the level of expression of a given miR in each cell type of the plurality of cells. Expression levels can be depicted in a matrix (e.g., a heatmap) with each miR represented as a row and each cell type or state represented as a column.

The level of expression is normalized such that the total level of expression among all the cell types or states in a row is set at a constant, e.g., 1. In the heatmap shown in the Examples, adding up all cells in any row would yield a total intensity value of 1.

For each of the plurality of cell types, the miRs are sorted from those that are more differentially expressed to those that are less differentially expressed. Moving down a column (e.g., cell type in the heatmap of the Examples), each cell has a higher value of normalized differential expression, hence, a higher intensity, than the cell in the adjacent row directly below. Following successive rows downwards, the intensity becomes more evenly distributed amongst the six cell types.

In this analysis, miRs that top the matrix are non-uniformly expressed among the plurality of cell types. Non-uniformity can be attributed to concentration of expression in one particular cell type (the target cell type), i.e., expression in one cell type and not in the other cell types in the plurality, (e.g., not in 2, 3, 4, or 5 cell types). There are less frequent cases where, e.g., normalized expression is high not only for the target cell type, but also for another cell type among the remaining cell types in the plurality. In those cases, entropy would be lower, but that non-uniformity is a result of concentration of expression in more than one cell type. For example, a given miR may be preferentially expressed in 2 cell types, but not in 3, 4, or 5 cell types across the plurality of cells.

The level of uniformity/Shannon entropy is calculated to quantify and affirm the sorting of miRs that are more differentially expressed from those that are more uniformly expressed across the plurality of cells.

Entropy. S, is calculated as shown below where Pᵢ represents the normalized expression of the miR in cell type i, where i is a particular cell type or state {e.g., where I is a B-cell, DC, T-cell, Bone marrow, Macrophage, Monocyte}. Σ, is taken over all of the plurality of cells, e.g., over all 6 cell types.

$$S = - {\sum }_{i} {P}_{i} log {P}_{i}$$

miRs selected for their non-uniformity of expression in this way can be tested for their ability to reduce expression of mRNAs in each of the plurality of cell types and should preferentially reduce expression in the cell type or state in which they are preferentially expressed.

In some embodiments, a miR of the disclosure is differentially expressed in a type of immune cells comprising B cells. In some embodiments, a miR is differentially expressed by a type of immune cells comprising B cells compared to additional types of immune cells selected from a group consisting of: T cells, macrophages, monocytes, dendritic cells, bone marrow cells, macrophages, neutrophils, and NK cells. In some embodiments, a miR is differentially expressed by a type of immune cells comprising B cells compared to a type of immune cells comprising T cells, a type of immune cells comprising monocytes, a type of immune cells comprising macrophages, a type of immune cells comprising dendritic cells, and a type of immune cells comprising bone marrow cells. In some embodiments, a miR that is differentially expressed by a type of immune cells comprising B cells is selected from a group identified in Table 1.

In some embodiments, a miR of the disclosure is differentially expressed in a type of immune cells comprising T cells. In some embodiments, a miR is differentially expressed by a type of immune cells comprising T cells compared to additional types of immune cells selected from a group consisting of: B cells, macrophages, monocytes, dendritic cells, bone marrow cells, macrophages, neutrophils, and NK cells. In some embodiments, a miR is differentially expressed by a type of immune cells comprising T cells compared to a type of immune cells comprising B cells, a type of immune cells comprising monocytes, a type of immune cells comprising macrophages, a type of immune cells comprising dendritic cells, and a type of immune cells comprising bone marrow cells. In some embodiments, a miR that is differentially expressed by a type of immune cells comprising T cells is selected from a group identified in Table 2.

In some embodiments, a miR of the disclosure is differentially expressed in a type of immune cells comprising DCs. In some embodiments, a miR is differentially expressed by a type of immune cells comprising DCs cells compared to additional types of immune cells selected from a group consisting of: B cells, T cells, macrophages, monocytes, bone marrow cells, macrophages, neutrophils, and NK cells. In some embodiments, a miR is differentially expressed by a type of immune cells comprising DCs compared to a type of immune cells comprising T cells, a type of immune cells comprising monocytes, a type of immune cells comprising macrophages, a type of immune cells comprising B cells, and a type of immune cells comprising bone marrow cells. In some embodiments, a miR that is differentially expressed by a type of immune cells comprising DCs is selected from a group identified in Table 3.

In some embodiments, a miR of the disclosure is differentially expressed in a type of immune cells comprising monocytes. In some embodiments, a miR is differentially expressed by a type of immune cells comprising monocytes compared to additional types of immune cells selected from a group consisting of: T cells, B cells, macrophages, dendritic cells, bone marrow cells, macrophages, neutrophils, and NK cells. In some embodiments, a miR is differentially expressed by a type of immune cells comprising monocytes compared to a type of immune cells comprising T cells, a type of immune cells comprising B cells, a type of immune cells comprising macrophages, a type of immune cells comprising dendritic cells, and a type of immune cells comprising bone marrow cells. In some embodiments, a miR that is differentially expressed by a type of immune cells comprising monocytes is selected from a group identified in Table 4.

In some embodiments, a miR of the disclosure is differentially expressed in a type of immune cells comprising macrophages. In some embodiments, a miR is differentially expressed by a type of immune cells comprising macrophages compared to additional types of immune cells selected from a group consisting of: T cells, B cells, monocytes, dendritic cells, bone marrow cells, macrophages, neutrophils, and NK cells. In some embodiments, a miR is differentially expressed by a type of immune cells comprising macrophages compared to a type of immune cells comprising T cells, a type of immune cells comprising monocytes, a type of immune cells comprising B cells, a type of immune cells comprising dendritic cells, and a type of immune cells comprising bone marrow cells. In some embodiments, a miR that is differentially expressed by a type of immune cells comprising macrophages is selected from a group identified in Table 5.

In some embodiments, a miR of the disclosure is differentially expressed in a type of immune cells comprising bone marrow cells. In some embodiments, a miR is differentially expressed by a type of immune cells comprising bone marrow cells compared to additional types of immune cells selected from a group consisting of: T cells, macrophages, monocytes, dendritic cells, B cells, macrophages, neutrophils, and NK cells. In some embodiments, a miR is differentially expressed by a type of immune cells comprising bone marrow cells compared to a type of immune cells comprising T cells, a type of immune cells comprising monocytes, a type of immune cells comprising macrophages, a type of immune cells comprising dendritic cells, and a type of immune cells comprising B cells. In some embodiments, a miR that is differentially expressed by a type of immune cells comprising bone marrow cells is selected from a group identified in Table 6.

microRNAs Differentially Expressed in Immune Cells

TABLE 1
B cell miRs

miR-1253
miR-486-3p
miR-452-5p
miR-3144-3p
miR-151a-5p
miR-1264
miR-548ah-5p
miR-1276
miR-1275
miR-502-5p
miR-520g-3p
miR-25-5p
miR-888-5p
miR-4488
miR-6724-5p
miR-222-3p
miR-506-3p
miR-4531
miR-1972
miR-1298-5p
miR-548m
miR-890
miR-195-5p
miR-202-3p
miR-129-5p
miR-577
miR-374c-5p
miR-33a-5p
miR-548j-3p
miR-4707-3p

TABLE 2
T cell miRs

miR-31-5p
miR-194-5p
miR-190b
miR-664b-5p
miR-542-5p
miR-525-3p
miR-561-3p
miR-1281
miR-450b-5p
miR-510-5p
miR-1249-5p
miR-514b-3p
miR-758-5p
miR-409-5p
miR-1245b-3p
miR-1260b
miR-1289
miR-485-5p
miR-620
miR-1306-3p
miR-708-5p
miR-1272
miR-196a-3p
miR-1908-3p
miR-641
miR-92a-3p
miR-101-3p
miR-520e
miR-301a-5p
miR-182-3p

TABLE 3
Dendritic cells miRs

miR-494-3p
miR-221-3p
miR-132-3p
miR-128-3p
miR-99b-5p
miR-34a-5p
let-7e-5p
miR-125a-5p
miR-21-5p
miR-99a-5p
miR-365a-3p + miR-365b-3p
miR-503-5p
miR-324-5p
miR-424-5p
miR-1249-3p
miR-23a-3p
miR-378i
let-7d-5p
miR-532-5p
miR-223-3p
miR-185-5p
miR-155-5p
miR-340-5p
miR-511-5p
miR-107
miR-98-5p
miR-362-3p
miR-191-5p
miR-24-3p
miR-660-5p

TABLE 4
Monocyte miRs

miR-570-3p
miR-924
miR-603
miR-2682-5p
miR-630
miR-597-5p
miR-4755-5p
miR-508-3p
miR-3144-3p
miR-604
miR-5481
miR-589-5p
miR-543
miR-3140-3p
miR-181a-5p
miR-151b
miR-506-5p
miR-371b-5p
miR 4286
miR-548z + miR548h-3p
miR-548k
miR-1245b-5p
miR-568
miR-181a-2-3p
miR-548h-5p
miR-526b-5p
miR-548aa + miR-548t-3p
miR-526a + miR-518c-5p + miR-518d-5p
miR-525-5p
miR-4454 + miR-7975

TABLE 5
Macrophage miRs

miR-769-3p
miR-181d-3p
miR-599
miR-566
miR-876-5p
miR-1296-5p
miR-33b-5p
miR-1291
miR-133a-3p
miR-514a-5p
miR-571
miR-639
miR-517a-3p
miR-3180-3p
miR-487b-5p
miR-510-3p
miR-346
miR-4431
miR-375
miR-224-5p
miR-508-5p
miR-1205
miR-550a-5p
miR-525-3p
miR-548al
miR-128-2-5p
miR-5010-5p
miR-1228-3p
miR-513c-3p
miR-552-3p

TABLE 6
Bone marrow cell miRs

miR-144-3p
miR-451a
miR 126-3p
miR-148a-3p
miR-199b-5p
miR-335-5p
miR-146b-5p
miR-130a-3p
miR-181a-3p
miR-223-3p
miR-652-3p
miR-301a-3p
miR-146a-5p
miR-27b-3p
miR-340-5p
miR-450a-5p
miR-133b
miR-342-5p
miR-19a-3p
miR-150-5p
miR-20a-5p + miR-20b-5p
miR-374a-5p
miR-582-5p
miR-16-5p
miR-660-5p
miR-93-5p
miR-1247-5p
miR-125b-5p
let-7g-5p
miR-25-3p

In some embodiments, a miR of the disclosure is expressed by a type of immune cells. In some embodiments, a miR of the disclosure is abundantly expressed, e.g., significantly expressed, by a type of immune cells. In some embodiments, an abundantly expressed miR is the first, second, third, fourth, fifth, sixth, seventh, eighth, ninth, tenth, eleventh, twelfth, thirteenth, fourteenth, or fifteenth most highly expressed miR by a type of immune cells. In some embodiments, an abundantly expressed miR is the first most highly expressed miR by a type of immune cells. In some embodiments, an abundantly expressed miR is the second most highly expressed miR by a type of immune cells. In some embodiments, an abundantly expressed miR is the third most highly expressed miR by a type of immune cells. In some embodiments, an abundantly expressed miR is the fourth most highly expressed miR by a type of immune cells. In some embodiments, an abundantly expressed miR is the fifth most highly expressed miR by a type of immune cells. In some embodiments, an abundantly expressed miR is the sixth most highly expressed miR by a type of immune cells.

In some embodiments, the abundance of a given miR is measured as a proportion of the total miRs expressed by a type of immune cells. In some embodiments, an abundantly expressed miR is at least about 0.5-5%, 1-5%, 1-10%, 2-10%, 2-20%, 3-10%, 3-20%, 3-30%, 4-10%, 4-20%, 4-30%, 4-40%, 5-10%, 5-20%, 5-30%, 5-40%, or 5-50% of all miRs expressed by a type of immune cells. In some embodiments, an abundantly expressed miR is at least about 1%, at least about 2%, at least about 3%, at least about 4%, at least about 5%, at least about 6%, at least about 7%, at least about 8%, at least about 9%, at least about 10%, at least about 15%, at least about 20%, at least about 30%, at least about 40%, or at least about 50% of all miRs expressed by a type of immune cells.

In some embodiments, a miR of the disclosure is abundantly expressed by a first type of immune cells, a second type of immune cells, a third type of immune cells, a fourth type of immune cells, and a fifth type of immune cells. In some embodiment, a first type of immune cells comprises T cells, a second type of immune cells comprises B cells, a third type of immune cells comprises monocytes, a fourth type of immune cells comprises macrophages, and a fifth type of immune cells comprises DCs. In some embodiments, a miR that is abundantly expressed by a first type of immune cells, a second type of immune cells, a third type of immune cells, a fourth type of immune cells, and a fifth type of immune cells is miR-142-3p. In some embodiments, a miR that is abundantly expressed by immune cells comprising T cells, B cells, monocytes, macrophages, and DCs is miR-142-3p.

In some embodiments, a miR of the disclosure is abundantly expressed by a first type of immune cells, a second type of immune cells, a third type of immune cells, and a fourth type of immune cells, but not abundantly expressed by a fifth type of immune cells. In some embodiment, a first type of immune cells comprises T cells, a second type of immune cells comprises B cells, a third type of immune cells comprises monocytes, a fourth type of immune cells comprises macrophages, and a fifth type of immune cells comprises DCs. In some embodiments, a miR that is abundantly expressed by a first type of immune cells, a second type of immune cells, a third type of immune cells, a fourth type of immune cells, but not abundantly expressed by a fifth type of immune cells is miR-150-3p. In some embodiments, a miR that is abundantly expressed by a first type of immune cells, a second type of immune cells, a third type of immune cells, a fourth type of immune cells, but not abundantly expressed by a fifth type of immune cells, is miR-29b-3p. In some embodiments, a miR that is abundantly by immune cells comprising T cells, B cells, monocytes, and macrophages, but not abundantly expressed by DCs, is miR-150-3p or miR-29b-3p.

In some embodiments, a miR of the disclosure is abundantly expressed by a first type of immune cells, a second type of immune cells, a third type of immune cells, and a fourth type of immune cells, but not abundantly expressed by a fifth type of immune cells. In some embodiment, a first type of immune cells comprises B cells, a second type of immune cells comprises monocytes, a third type of immune cells comprises macrophages, a fourth type of immune cells comprises DCs, and a fifth type of immune cells comprises T cells. In some embodiments, a miR that is abundantly expressed by a first type of immune cells, a second type of immune cells, a third type of immune cells, a fourth type of immune cells, but not abundantly expressed by a fifth type of immune cells is miR-16a-5p. In some embodiments, a miR that is abundantly expressed by immune cells comprising B cells, monocytes, macrophages and DCs, but not abundantly expressed by T cells, is miR-16a-5p.

In some embodiments, a miR of the disclosure is abundantly expressed by a first type of immune cells, a second type of immune cells, and a third type of immune cells, but not abundantly expressed by a fourth type of immune cells or a fifth type of immune cells.

In some embodiments, a miR of the disclosure is abundantly expressed by a first type of immune cells and a second type of immune cells, but not abundantly expressed by a third type of immune cells, a fourth type of immune cells, or a fifth type of immune cells. In some embodiment, a first type of immune cells comprises monocytes, a second type of immune cells comprises macrophages, a third type of immune cells comprises B cells, a fourth type of immune cells comprises T cells, and a fifth type of immune cells comprises DCs. In some embodiments, a miR that is abundantly expressed by a first type of immune cells and a second type of immune cells, but not abundantly expressed by a third type of immune cells, a fourth type of immune cells, or a fifth type of immune cells is miR-4454 or miR-7975. In some embodiments, a miR that is abundantly expressed by immune cells comprising monocytes and macrophages, but not abundantly expressed by B cells, T cells, or DCs is miR-4454 or miR-7975.

In some embodiments, a miR of the disclosure is abundantly expressed by a first type of immune cells and a second type of immune cells, but not abundantly expressed by a third type of immune cells, a fourth type of immune cells, or a fifth type of immune cells. In some embodiment, a first type of immune cells comprises B cells, a second type of immune cells comprises T cells, a third type of immune cells comprises monocytes, a fourth type of immune cells comprises macrophages, and a fifth type of immune cells comprises DCs. In some embodiments, a miR that is abundantly expressed by a first type of immune cells and a second type of immune cells, but not abundantly expressed by a third type of immune cells, a fourth type of immune cells, or a fifth type of immune cells is let7g-5p. In some embodiments, a miR that is abundantly expressed by immune cells comprising B cells and T cells, but not abundantly expressed by macrophages, monocytes, or DCs is let7g-5p.

In some embodiments, a miR of the disclosure is abundantly expressed by a first type of immune cells, but not abundantly expressed by a second type of immune cells, a third type of immune cells, a fourth type of immune cells, or a fifth type of immune cells. In some embodiment, a first type of immune cells comprises T cells, a second type of immune cells comprises B cells, a third type of immune cells comprises macrophages, a fourth type of immune cells comprises monocytes, and a fifth type of immune cells comprises DCs. In some embodiments, a miR that is abundantly expressed by a first type of immune cells, but not abundantly expressed by a second type of immune cells, a third type of immune cells, a fourth type of immune cells, or a fifth type of immune cells is miR-342-3p. In some embodiments, a miR that is abundantly expressed by immune cells comprising T cells, but not abundantly expressed by B cells, macrophages, monocytes or DCs is miR-342-3p.

In some embodiments, a miR of the disclosure is abundantly expressed by a first type of immune cells, but not abundantly expressed by a second type of immune cells, a third type of immune cells, a fourth type of immune cells, or a fifth type of immune cells. In some embodiment, a first type of immune cells comprise DCs, a second type of immune cells comprises T cells, a third type of immune cells comprises B cells, a fourth type of immune cells comprises macrophages, and a fifth type of immune cells comprises monocytes. In some embodiments, a miR that is abundantly expressed by a first type of immune cells, but not abundantly expressed by a second type of immune cells, a third type of immune cells, a fourth type of immune cells, or a fifth type of immune cells is selected from a group consisting of: miR-21-5p miR-223-3p, or let7a-5p. In some embodiments, a miR that is abundantly expressed by immune cells comprising DCs, but not abundantly expressed by T cells, B cells, macrophages or monocytes is selected from a group consisting of: miR-21-5p, miR-223-3p, or let7a-5p.

### Cancer Related MicroRNAs

In some embodiments, a miR of the disclosure is expressed by a type of cancer cells (e.g., AML cells). In some embodiments, a miR of the disclosure is abundantly expressed by a type of cancer cells (e.g., AML cells). In some embodiments, an abundantly expressed miR is the first, second, third, fourth, fifth, sixth, seventh, eighth, ninth, tenth, eleventh, twelfth, thirteenth, fourteenth, or fifteenth most highly expressed miR by a type of cancer cells (e.g., AML cells). In some embodiments, an abundantly expressed miR is the first most highly expressed miR by a type of cancer cells (e.g., AML cells). In some embodiments, an abundantly expressed miR is the second most highly expressed miR by a type of cancer cells (e.g., AML cells). In some embodiments, an abundantly expressed miR is the third most highly expressed miR by a type of cancer cells (e.g., AML cells). In some embodiments, an abundantly expressed miR is the fourth most highly expressed miR by a type of cancer cells (e.g., AML cells). In some embodiments, an abundantly expressed miR is the fifth most highly expressed miR by a type of cancer cells (e.g., AML cells). In some embodiments, an abundantly expressed miR is the sixth most highly expressed miR by a type of cancer cells (e.g., AML cells).

In some embodiments, the abundance of a given miR is measured as a proportion of the total miRs expressed by a type of cancer cells (e.g., AML cells). In some embodiments, an abundantly expressed miR is at least about 0.5-5%, 1-5%, 1-10%, 2-10%, 2-20%, 3-10%, 3-20%, 3-30%, 4-10%, 4-20%, 4-30%, 4-40%, 5-10%, 5-20%, 5-30%, 5-40%, or 5-50% of all miRs expressed by a type of cancer cells (e.g., AML cells). In some embodiments, an abundantly expressed miR is at least about 1%, at least about 2%, at least about 3%, at least about 4%, at least about 5%, at least about 6%, at least about 7%, at least about 8%, at least about 9%, at least about 10%, at least about 15%, at least about 20%, at least about 30%, at least about 40%, or at least about 50% of all miRs expressed by a type of cancer cells (e.g., AML cells).

In some embodiments, a miR of the disclosure is abundantly expressed by a type of cancer cell. In some embodiments, a miR of the disclosure is abundantly expressed by a type of cancer cells comprising AML cells. In some embodiments, a miR that is abundantly expressed by a type of cancer cells comprising AML cells is selected from a group shown in Table 7.

TABLE 7
microRNAs Abundantly Expressed in AML Cells
AML miRs

miR-7a-5p
miR-7b-5p
let-7a-5p
let-7b-5p
miR-15b-5p
miR-16-5p
miR-18a-5p
miR-19b-3p
miR-20a-5p + miR-20b-5p
miR-106a-5p + miR-17-5p
miR-126-3p
miR-142-3p
miR-181a-5p
miR-191-5p
miR-223-3p

In some embodiments, a miR of the disclosure is differentially expressed in a type of immune cancer cells (e.g., AML cells) relative to a plurality of other cell types, e.g., types of non-cancerous immune cells (e.g., healthy cells). The differential expression of a miR in a type of cancer cells (e.g., AML cells) relative to a type of non-cancerous cells (e.g., healthy cells) is determined as defined herein. In some embodiments, the miRs are also abundantly expressed, e.g., demonstrate increased expression of a miR in a type of cancer cells (e.g., AML cells) is at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, or at least 15-fold higher than expression in a type of non-cancerous cells (e.g., healthy cells). In some embodiments, increased expression of a miR in a type of cancer cells (e.g., AML cells) is at least 10-fold higher than expression in a type of non-cancerous cells (e.g., healthy cells).

In some embodiments, a miR of the disclosure is differentially expressed in type of immune cancer cells comprising AML cells relative to a type of non-cancerous immune cells. In some embodiments, a miR of the disclosure is differentially expressed in type of cancer cells comprising AML cells relative to a type of non-cancerous cells comprising any type of immune cell. In some embodiments, a miR of the disclosure is differentially expressed in a type of cancer cells comprising AML cells relative to a type of non-cancerous cells comprising any one of bone marrow cells, B cells, macrophages, T cells, monocytes, or a combination thereof. In some embodiments, a miR that is differentially expressed in a type of cancer cells comprising AML cells relative to a type of non-cancerous cells is miR-18a-5p. In some embodiments, a miR that is differentially expressed in a type of cancer cells comprising AML cells relative to a type of non-cancerous cells is a mature miR derived from miR-1246. In some embodiments, a miR that is differentially expressed in a type of cancer cells comprising AML cells relative to a type of non-cancerous cells is miR-126-3p.

In some embodiments, a miR of the disclosure is differentially expressed in a type of non-cancerous immune cells (e.g., healthy cells) relative to a type of immune cancer cells (e.g., AML cells). The differential expression of a miR in a type of non-cancerous cells (e.g., healthy cells) relative to a type of cancer cells (e.g., AML cells) refers to the increased expressed of the miR in a given type of non-cancerous cells (e.g., healthy cells) compared to a given type or plurality of cancerous cells (e.g., AML cells). In some embodiments, increased expression of a miR in a type of non-cancerous cells (e.g., healthy cells) is at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, or at least 15-fold higher than expression in a type of cancerous cells (e.g., AML cells). In some embodiments, increased expression of a miR in a type of non-cancerous cells (e.g., healthy cells) is at least 10-fold higher than expression in a type of cancerous cells (e.g., AML cells).

In some embodiments, a miR of the disclosure is differentially expressed in type of non-cancerous immune cells relative to a type of immune cancer cells comprising AML cells. In some embodiments, a miR of the disclosure is differentially expressed in a type of non-cancerous cells comprising any type of immune cell relative to a type of cancer cells comprising AML cells. In some embodiments, a miR of the disclosure is differentially expressed in a type of non-cancerous cells comprising any one of bone marrow cells, B cells, macrophages, T cells, monocytes, or a combination thereof relative to a type of cancer cells comprising AML cells. In some embodiments, a miR that is differentially expressed in a type of non-cancerous cells comprising immune cells relative to a type of cancer cells comprising AML cells is any one selected from a group consisting of: miR-150-5p, miR-146-5p, miR-4286, miR-579-3p, miR-4516, miR-146a-5p, miR-664b-3p, miR-342-3p, miR-1915-3p, or miR-26b-5p.

### Immune Cell Subset MicroRNAs

In some embodiments, a miR of the disclosure is expressed by a type of immune cells (e.g., naïve T cells, effector T cells, regulatory T cells, activated T cells). In some embodiments, a miR of the disclosure is abundantly expressed by a type of immune cells (e.g., naïve T cells, effector T cells, regulatory T cells, activated T cells). In some embodiments, an abundantly expressed miR is the first, second, third, fourth, fifth, sixth, seventh, eighth, ninth, tenth, eleventh, twelfth, thirteenth, fourteenth, or fifteenth most highly expressed miR by a type of immune cells (e.g., naïve T cells, effector T cells, regulatory T cells, activated T cells). In some embodiments, an abundantly expressed miR is the first most highly expressed miR by a type of immune cells (e.g., naïve T cells, effector T cells, regulatory T cells, activated T cells). In some embodiments, an abundantly expressed miR is the second most highly expressed miR by a type of immune cells (e.g., naïve T cells, effector T cells, regulatory T cells, activated T cells). In some embodiments, an abundantly expressed miR is the third most highly expressed miR by a type of immune cells (e.g., naïve T cells, effector T cells, regulatory T cells, activated T cells). In some embodiments, an abundantly expressed miR is the fourth most highly expressed miR by a type of immune cells (e.g., naïve T cells, effector T cells, regulatory T cells, activated T cells). In some embodiments, an abundantly expressed miR is the fifth most highly expressed miR by a type of immune cells (e.g., naïve T cells, effector T cells, regulatory T cells, activated T cells). In some embodiments, an abundantly expressed miR is the sixth most highly expressed miR by a type of immune cells (e.g., naïve T cells, effector T cells, regulatory T cells, activated T cells).

In some embodiments, the abundance of a given miR is measured as a proportion of the total miRs expressed by a type of immune cells (e.g., naïve T cells, effector T cells, regulatory T cells, activated T cells). In some embodiments, an abundantly expressed miR is at least about 0.5-5%, 1-5%, 1-10%, 2-10%, 2-20%, 3-10%, 3-20%, 3-30%, 4-10%, 4-20%, 4-30%, 4-40%, 5-10%, 5-20%, 5-30%, 5-40%, or 5-50% of all miRs expressed by a type of immune cells (e.g., naïve T cells, effector T cells, regulatory T cells, activated T cells). In some embodiments, an abundantly expressed miR is at least about 1%, at least about 2%, at least about 3%, at least about 4%, at least about 5%, at least about 6%, at least about 7%, at least about 8%, at least about 9%, at least about 10%, at least about 15%, at least about 20%, at least about 30%, at least about 40%, or at least about 50% of all miRs expressed by a type of immune cells (e.g., naïve T cells, effector T cells, regulatory T cells, activated T cells).

In some embodiments, a miR of the disclosure is abundantly expressed by a type of immune cells (e.g., naïve T cells, effector T cells, or regulatory T cells). In some embodiments, a miR of the disclosure is abundantly expressed by a type of immune cells comprising naïve T cells. In some embodiments, a miR that is abundantly expressed by a type of immune cells comprising naïve T cells is selected from a group consisting of: miR-150-5p, miR-142-3p, miR-342-3p, let-7g-5p, or miR-29b-3p. In some embodiments, a miR of the disclosure is abundantly expressed by a type of immune cells comprising effector T cells. In some embodiments, a miR that is abundantly expressed by a type of immune cells comprising effector T cells is selected from a group consisting of: miR-150-5p, miR-142-3p, miR-29b-3p, miR-342-3p, let-7g-5p. In some embodiments, a miR of the disclosure is abundantly expressed by a type of immune cells comprising regulatory T cells. In some embodiments, a miR that is abundantly expressed by a type of immune cells comprising regulator T cells is selected from a group consisting of: miR-150-5p, miR-142-3p, miR-29b-3p, miR-146a-5p, or miR-223-3p.

In some embodiments, a miR of the disclosure is abundantly expressed by a type of immune cells (e.g., activated CD3+ T cells or resting CD3+ T cells). In some embodiments, a miR of the disclosure is abundantly expressed by a type of immune cells comprising resting CD3+ T cells. In some embodiments, a miR that is abundantly expressed by a type of immune cells comprising CD3+ T cells is selected from a group consisting of: miR-150-5p, miR-142-3p, miR-29b-3p, let-7g-5p, or miR-342-3p. In some embodiments, a miR of the disclosure is abundantly expressed by a type of immune cells comprising activated CD3+ T cells. In some embodiments, a miR that is abundantly expressed by a type of immune cells comprising CD3+ T cells is selected from a group consisting of: miR-150-5p, miR-142-3p, miR-29b-3p, miR-342-3p, let-7g-5p, or miR-155-5p.

In some embodiments, a miR of the disclosure is abundantly expressed by a type of immune cells (e.g., activated CD3+CD4+ T cells or resting CD3+CD4+ T cells). In some embodiments, a miR of the disclosure is abundantly expressed by a type of immune cells comprising resting CD3+CD4+ T cells. In some embodiments, a miR that is abundantly expressed by a type of immune cells comprising resting CD3+CD4+ T cells is selected from a group consisting of: miR-150-5p, miR-142-3p, miR-29b-3p, let-7g-5p, or miR-342-3p. In some embodiments, a miR of the disclosure is abundantly expressed by a type of immune cells comprising activated CD3+ CD4+ T cells. In some embodiments, a miR that is abundantly expressed by a type of immune cells comprising activated CD3+ CD4+ T cells is selected from a group consisting of: miR-150-5p, miR-142-3p, miR-29b-3p, miR-342-3p, let-7g-5p, or miR-155-5p.

In some embodiments, a miR of the disclosure is abundantly expressed by a type of immune cells (e.g., activated CD3+CD3+ T cells or resting CD3+CD8+ T cells). In some embodiments, a miR of the disclosure is abundantly expressed by a type of immune cells comprising resting CD3+CD8+ T cells. In some embodiments, a miR that is abundantly expressed by a type of immune cells comprising resting CD3+CD8+ T cells is selected from a group consisting of: miR-150-5p, miR-142-3p, miR-29b-3p, let-7g-5p. miR-342-3p. In some embodiments, a miR of the disclosure is abundantly expressed by a type of immune cells comprising activated CD3+CD8+ T cells. In some embodiments, a miR that is abundantly expressed by a type of immune cells comprising activated CD3+CD8+ T cells is selected from a group consisting of: miR-150-5p, miR-142-3p, miR-29b-3p, miR-342-3p, let-7g-5p, miR-155-5p, miR-4454, or miR-7975.

In some embodiments, a miR of the disclosure is differentially expressed in a type of immune cells comprising regulatory T cells relative to a plurality of other types of immune cells, e.g., naïve T cells or effector T cells. The differential expression of a miR in regulatory T cells relative to naïve T cells or effector T is determined by looking at differential expression of the miR in regulatory T cells compared to naïve T cells or effector T cells as defined herein. In some embodiments, the miR is also abundantly expressed, e.g., increased expression of the miR in a type of immune cells comprising regulatory T cells is at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, or at least 15-fold higher than expression of the miR in a type of immune cells comprising naïve T cells or effector T cells. In some embodiments, increased expression of a miR in a type of immune cells comprising regulatory T cells is at least 10-fold higher than expression of the miR in a type of immune cells comprising naïve T cells or effector T cells.

In some embodiments, a miR of the disclosure is differentially expressed in type of immune cells comprising regulatory T cells relative to a plurality of immune cells of different activation states, e.g., comprising activation and naïve T cells. In some embodiments, a miR that is differentially expressed in a type of immune cell comprising regulatory T cells relative to a type of immune cell comprising naïve T cells is selected from a group consisting of: miR-146a-5p, miR-21-5p, miR-155-5p, miR-15a-5p, let-7i-5p, miR-16-5p, miR-222-3p, miR-15b-5p, miR-24-3p, or miR-4443. In some embodiments, a miR that is differentially expressed in a type of immune cell comprising regulatory T cells relative to a type of immune cell comprising naïve T cells is miR-146a-5p.

In some embodiments, a miR of the disclosure is differentially expressed in type of immune cells comprising regulatory T cells relative to a plurality of immune cells of different states, e.g., comprising effector T cells and naïve T cells. In some embodiments, a miR that is differentially expressed in a type of immune cell comprising regulatory T cells relative to a type of immune cell comprising effector T cells is selected from a group consisting of: miR-146a-5p, miR-181a-5p, miR-223-3p, miR-15a-5p, miR-4286, miR-378g, miR-93-5p, miR-16-5p, miR-25-3p, or miR-15b-p. In some embodiments, a miR that is differentially expressed in a type of immune cell comprising regulatory T cells relative to a type of immune cell comprising effector T cells is miR-146a-5p.

In some embodiments, a miR of the disclosure is differentially expressed in a type of immune cells comprising activated T cells relative to a plurality of immune cells comprising immune cells of different activation states, e.g., resting and regulatory T cells. The differential expression of a miR in activated T cells relative to resting T cells refers to the increased expressed of the miR in activated T cells compared to resting T cells. In some embodiments, increased expression of a miR in a type of immune cells comprising activated T cells is at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, or at least 15-fold higher than expression of the miR in a type of immune cells comprising resting T cells. In some embodiments, increased expression of a miR in a type of immune cells comprising activated T cells is at least 10-fold higher than expression of the miR in a type of immune cells comprising resting T cells.

In some embodiments, a miR of the disclosure is differentially expressed in type of immune cells comprising activated CD3+ T cells relative to a plurality of immune cells comprising resting CD3+ T cells. In some embodiments, a miR that is differentially expressed in a type of immune cell comprising activated CD3+ T cells relative to a type of immune cell comprising resting CD3+ T cells is selected from a group consisting of: miR-155-5p, miR-132-3p, miR-106a-5p, miR-17-5p, miR-19b-3p, miR-19a-3p, miR-24-3p, miR-20a-5p, miR-20b-5p, miR-29a-3p, miR-98-5p, or miR-342-5p. In some embodiments, a miR that is differentially expressed in a type of immune cell comprising activated CD3+ T cells relative to a type of immune cell comprising resting CD3+ T cells is miR-155-5p. In some embodiments, a miR that is differentially expressed in a type of immune cell comprising activated CD3+ T cells relative to a type of immune cell comprising resting CD3+ T cells is miR-132-3p.

In some embodiments, a miR of the disclosure is differentially expressed in type of immune cells comprising activated CD3+CD4+ T cells relative to a plurality of immune cells comprising resting CD3+CD4+ T cells. In some embodiments, a miR that is differentially expressed in a type of immune cell comprising activated CD3+CD4+ T cells relative to a type of immune cell comprising resting CD3+CD4+ T cells is selected from a group consisting of: miR-155-5p, miR-132-3p, miR-106a-5p, miR-17-5p, miR-20a-5p, miR-20b-5p, miR-98-5p, miR-19b-3p, miR-19a-3p, miR-4454, miR-7975, miR-92a-3p, or miR-29a-3p. In some embodiments, a miR that is differentially expressed in a type of immune cell comprising activated CD3+CD4+ T cells relative to a type of immune cell comprising resting CD3+CD4+ T cells is miR-155-5p. In some embodiments, a miR that is differentially expressed in a type of immune cell comprising activated CD3+CD4+ T cells relative to a type of immune cell comprising resting CD3+CD4+ T cells is miR-132-3p.

In some embodiments, a miR of the disclosure is differentially expressed in type of immune cells comprising activated CD3+CD8+ T cells relative to a plurality of immune cells comprising resting CD3+CD8+ T cells. In some embodiments, a miR that is differentially expressed in a type of immune cell comprising activated CD3+CD8+ T cells relative to a type of immune cell comprising resting CD3+CD8+ T cells is selected from a group consisting of: miR-155-5p, miR-132-3p, miR-106a-5p, miR-17-5p, miR-20a-5p, miR-20b-5p, miR-4454, miR-7975, let-7a-5p, miR-19a-3p, miR-19b-3p, miR-4443, miR-29b-3p. In some embodiments, a miR that is differentially expressed in a type of immune cell comprising activated CD3+CD8+ T cells relative to a type of immune cell comprising resting CD3+CD8+ T cells is miR-155-5p. In some embodiments, a miR that is differentially expressed in a type of immune cell comprising activated CD3+CD8+ T cells relative to a type of immune cell comprising resting CD3+CD8+ T cells is miR-132-3p.

### MicroRNA Binding Sites

In some embodiments, an mRNA of the present disclosure, comprising an open reading frame (ORF) encoding a polypeptide of interest, comprises one or more microRNA binding sites. Inclusion or incorporation of miR binding site(s) provides for regulation of mRNAs of the disclosure, and in turn, of the polypeptides encoded there from, based on tissue specific and/or cell type specific expression of naturally occurring miRs.

As used herein, the term “microRNA (miRNA or miR) binding site” refers to a sequence within a polynucleotide, e.g., within a DNA or within an RNA transcript (e.g., mRNA), including in the 5′UTR and/or 3′UTR, that has sufficient complementarity to all or a region of a miR to interact with, associate with or bind to the miR. In some embodiments, an mRNA of the disclosure comprising an ORF encoding a polypeptide of interest further comprises one or more miR binding site(s). In some embodiments, a 5′UTR and/or 3′UTR of the mRNA comprises the one or more miR binding site(s).

A miRNA binding site having sufficient complementarity to a miR refers to a degree of complementarity sufficient to facilitate miR-mediated regulation of an mRNA, e.g., miR-mediated translational repression or degradation of the mRNA. In some embodiments, a miR binding site having sufficient complementarity to the miR refers to a degree of complementarity sufficient to facilitate miR-mediated degradation of the mRNA, e.g., miR-guided RNA-induced silencing complex (RISC)-mediated cleavage of mRNA. The miR binding site can have complementarity to, for example, a 19-25 nucleotide miR sequence, to a 19-23 nucleotide miR sequence, or to a 22 nucleotide miR sequence. A miR binding site can be complementary to only a portion of a miR, e.g., to a portion less than 1, 2, 3, or 4 nucleotides of the full length of a naturally-occurring miR sequence. Full or complete complementarity (e.g., full complementarity or complete complementarity over all or a significant portion of the length of a naturally-occurring miR) is preferred when the desired regulation is mRNA degradation.

In some embodiments, the miR binding site is fully complementary to a miR, thereby degrading the mRNA fused to the miR binding site. In other embodiments, the miR binding site is not fully complementary to the corresponding miR. In certain embodiments, the miR binding site is the same length as the corresponding miR. In other embodiments, the miR binding site is one nucleotide shorter than the corresponding miR (e.g., a miR consisting of 19-25 nucleotides) at the 5′ terminus, the 3′ terminus, or both. In still other embodiments, the miR binding site is two nucleotides shorter than the corresponding miR at the 5′ terminus, the 3′ terminus, or both. In yet other embodiments, the miR binding site is three nucleotides shorter than the corresponding miR at the 5′ terminus, the 3′ terminus, or both. In some embodiments, the miR binding site is four nucleotides shorter than the corresponding miR at the 5′ terminus, the 3′ terminus, or both. In other embodiments, the miR binding site is five nucleotides shorter than the corresponding miR at the 5′ terminus, the 3′ terminus, or both. In some embodiments, the miR binding site is six nucleotides shorter than the corresponding miR at the 5′ terminus, the 3′ terminus, or both. In other embodiments, the miR binding site is seven nucleotides shorter than the corresponding miR at the 5′ terminus or the 3′ terminus. In other embodiments, the miR binding site is eight nucleotides shorter than the corresponding miR at the 5′ terminus or the 3′ terminus. In other embodiments, the miR binding site is nine nucleotides shorter than the corresponding miR at the 5′ terminus or the 3′ terminus. In other embodiments, the miR binding site is ten nucleotides shorter than the corresponding miR at the 5′ terminus or the 3′ terminus. In other embodiments, the miR binding site is eleven nucleotides shorter than the corresponding miR at the 5′ terminus or the 3′ terminus. In other embodiments, the miR binding site is twelve nucleotides shorter than the corresponding miR at the 5′ terminus or the 3′ terminus. The miR binding sites that are shorter than the corresponding miRs are still capable of degrading the mRNA incorporating one or more of the miR binding sites or preventing the mRNA from translation.

In some embodiments, the miR binding site is the same length as the corresponding miR. In other embodiments, the miR binding site is one, two, three, four, five, six, seven, eight, nine, ten, eleven or twelve nucleotide(s) shorter than the corresponding miR at the 5′ terminus, the 3′ terminus, or both. In still other embodiments, the miR binding site is two nucleotides shorter than the corresponding microRNA at the 5′ terminus, the 3′ terminus, or both. The miR binding sites that are shorter than the corresponding miRs are still capable of degrading the mRNA or preventing the mRNA from translation.

In some embodiments, the miR binding site binds the corresponding mature miR that is part of an active RISC containing Dicer. In another embodiment, binding of the miR binding site to the corresponding miR in RISC degrades the mRNA containing the miR binding site or prevents the mRNA from being translated. In some embodiments, the miR binding site has sufficient complementarity to miR so that a RISC complex comprising the miR cleaves the mRNA comprising the miR binding site. In other embodiments, the miR binding site has imperfect complementarity so that a RISC complex comprising the miR induces instability in the mRNA comprising the miR binding site. In another embodiment, the miR binding site has imperfect complementarity so that a RISC complex comprising the miR represses transcription of the mRNA comprising the miR binding site.

In some embodiments, the miR binding site has one, two, three, four, five, six, seven, eight, nine, ten, eleven or twelve mismatch(es) from the corresponding miR.

In some embodiments, the miR binding site has sufficient complementarity to miR so that a RISC complex comprising the miR cleaves the mRNA comprising the miR binding site. In other embodiments, the miR binding site has imperfect complementarity so that a RISC complex comprising the miR induces instability in the mRNA comprising the miR binding site. In another embodiment, the miR binding site has imperfect complementarity so that a RISC complex comprising the miR represses transcription of the mRNA comprising the miR binding site. In one embodiment, the miR binding site has one mismatch from the corresponding miR. In another embodiment, the miR binding site has two mismatches from the corresponding miR. In other embodiments, the miR binding site has three mismatches from the corresponding miR. In other embodiments, the miR binding site has four mismatches from the corresponding miR. In some embodiments, the miR binding site has five mismatches from the corresponding miR. In other embodiments, the miR binding site has six mismatches from the corresponding miR. In certain embodiments, the miR binding site has seven mismatches from the corresponding miR. In other embodiments, the miR binding site has eight mismatches from the corresponding miR. In other embodiments, the miR binding site has nine mismatches from the corresponding miR. In other embodiments, the miR binding site has ten mismatches from the corresponding miR. In other embodiments, the miR binding site has eleven mismatches from the corresponding miR. In other embodiments, the miR binding site has twelve mismatches from the corresponding miR.

In some embodiments, the miR binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miR.

In certain embodiments, the miR binding site has at least about ten contiguous nucleotides complementary to at least about ten contiguous nucleotides of the corresponding miR, at least about eleven contiguous nucleotides complementary to at least about eleven contiguous nucleotides of the corresponding miR, at least about twelve contiguous nucleotides complementary to at least about twelve contiguous nucleotides of the corresponding miR, at least about thirteen contiguous nucleotides complementary to at least about thirteen contiguous nucleotides of the corresponding miR, or at least about fourteen contiguous nucleotides complementary to at least about fourteen contiguous nucleotides of the corresponding miR. In some embodiments, the miR binding sites have at least about fifteen contiguous nucleotides complementary to at least about fifteen contiguous nucleotides of the corresponding miR, at least about sixteen contiguous nucleotides complementary to at least about sixteen contiguous nucleotides of the corresponding miR, at least about seventeen contiguous nucleotides complementary to at least about seventeen contiguous nucleotides of the corresponding miR, at least about eighteen contiguous nucleotides complementary to at least about eighteen contiguous nucleotides of the corresponding miR, at least about nineteen contiguous nucleotides complementary to at least about nineteen contiguous nucleotides of the corresponding miR, at least about twenty contiguous nucleotides complementary to at least about twenty contiguous nucleotides of the corresponding miR, or at least about twenty one contiguous nucleotides complementary to at least about twenty one contiguous nucleotides of the corresponding miR.

In some embodiments, a miR binding site includes a sequence that has complementarity (e.g., partial or complete complementarity) with a miR seed sequence. A miR “seed” sequence comprises a sequence in the region of approximately positions 2-8 of the mature miR. In some embodiments, a miR seed comprises positions 2-8 of the mature miR. In some embodiments, a miR seed comprises positions 2-7 of the mature miR. In some embodiments, a miR seed comprises 7 nucleotides (e.g., nucleotides 2-8 of the mature miR), wherein the seed-complementary site in the corresponding miR binding site is flanked by an adenosine (A) opposed to miR position 1. In some embodiments, a miR seed comprises 6 nucleotides (e.g., nucleotides 2-7 of the mature miRNA), wherein the seed-complementary site in the corresponding miR binding site is flanked by an adenosine (A) opposed to miR position 1. See, for example, Grimson A, Farh K K, Johnston W K, Garrett-Engele P, Lim L P, Bartel D P; Mol Cell. 2007 Jul. 6; 27(1):91-105. miR profiling of the target cells or tissues can be conducted to determine the presence or absence of miR in the cells or tissues. In some embodiments, an mRNA of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

In some embodiments, a miR binding site includes a sequence that has complementarity (e.g., partial or complete complementarity) with a miR seed sequence. In some embodiments, the miR binding site includes a sequence that has complete complementarity with a miR seed sequence. In some embodiments, a miR binding site includes a sequence that has complementarity (e.g., partial or complete complementarity) with an miR sequence. In some embodiments, the miR binding site includes a sequence that has complete complementarity with a miR sequence. In some embodiments, a miR binding site has complete complementarity with a miR sequence but for 1, 2, or 3 nucleotide substitutions, terminal additions, and/or truncations.

In some embodiments, an mRNA of the disclosure can be designed to incorporate miR binding sites that either have 100% identity to known miR seed sequences or have less than 100% identity to miR seed sequences. In some embodiments, an mRNA of the disclosure can be designed to incorporate miR binding sites that have at least: 60%, 65%, 70%, 75%, 80%. 85%, 90%, 95%, 96%, 97%, 98%, or 99% identity to known miR seed sequences. The miR seed sequence can be partially mutated to decrease miR binding affinity and as such result in reduced down-modulation of the mRNA. In essence, the degree of match or mis-match between the miR binding site and the miR seed can act as a rheostat to more finely tune the ability of the miR to modulate protein expression. In addition, mutation in the non-seed region of a miR binding site can also impact the ability of a miR to modulate protein expression.

### Location of miR Binding Site

In some embodiments, at least one miR binding site is inserted in the mRNA of the disclosure in any position of the molecule (e.g., the 5′UTR and/or 3′UTR). In some embodiments, the 5′UTR comprises at least one miR binding site. In some embodiments, the 3′UTR comprises at least one miR binding site. In some embodiments, the 5′UTR and the 3′UTR comprise at least one miR binding site. The insertion site of the miR binding site in the mRNA can be anywhere in the mRNA as long as the insertion of the miR binding site does not interfere with the translation of a functional polypeptide in the absence of the corresponding miR; and in the presence of the miR, the insertion of the miR binding and the binding of the miR binding site to the corresponding miR are capable of degrading the mRNA or preventing the translation of the mRNA.

In some embodiments, a miR binding site is inserted at least about 30 nucleotides downstream from the stop codon of an ORF in an mRNA of the disclosure comprising the ORF. In some embodiments, a miR binding site is inserted at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in an mRNA of the disclosure. In some embodiments, a miR binding site is inserted about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in an mRNA of the disclosure.

miR gene regulation can be influenced by the sequence surrounding the miR such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miR can be influenced by the 5′UTR and/or 3′UTR. As a non-limiting example, a non-human 3′UTR can increase the regulatory effect of the miR sequence on the expression of a polypeptide of interest compared to a human 3′UTR of the same sequence type.

In some embodiments, other regulatory elements and/or structural elements of the 5′UTR can influence miR mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miR mediated gene expression (Meijer H A et al., Science, 2013, 340, 82-85, herein incorporated by reference in its entirety). The mRNAs of the disclosure can further include this structured 5′UTR in order to enhance miR mediated gene regulation.

To further drive the selective degradation and suppression, an mRNA of the disclosure can include a further negative regulatory element in the 5′UTR and/or 3′UTR, either alone or in combination with miR binding sites. As a non-limiting example, the further negative regulatory element is a Constitutive Decay Element (CDE).

In some embodiments, at least one miR binding site is engineered into the 3′UTR of an mRNA of the disclosure. In this context, at least two, at least three, at least four, at least five, at least six, at least seven, at least eight, at least nine, at least ten, or more miR binding sites can be engineered into a 3′UTR of an mRNA of the disclosure. For example, 1 to 10, 1 to 9, 1 to 8, 1 to 7, 1 to 6, 1 to 5, 1 to 4, 1 to 3, 2, or 1 miR binding sites can be engineered into the 3′UTR of an mRNA of the disclosure. In some embodiments, miR binding sites incorporated into an mRNA of the disclosure can be the same or can be different miR binding sites. A combination of different miR binding sites incorporated into an mRNA of the disclosure can include combinations in which more than one copy of any of the different miR binding sites are incorporated. In another embodiment, miR binding sites incorporated into an mRNA of the disclosure can target the same or different tissues in the body. As a non-limiting example, through the introduction of tissue-, cell-type-, or disease-specific miR binding sites in the 3′-UTR of an mRNA of the disclosure, the degree of expression in specific cell types (e.g., B cells, T cells, dendritic cells, monocytes, macrophages, neutrophils, NK cells, cancer cells, etc.) can be reduced.

In some embodiments, a miR binding site is engineered near the 5′ terminus of the 3′UTR, about halfway between the 5′ terminus and 3′ terminus of the 3′UTR and/or near the 3′ terminus of the 3′UTR in an mRNA of the disclosure. As a non-limiting example, a miR binding site can be engineered near the 5′ terminus of the 3′UTR and about halfway between the 5′ terminus and 3′ terminus of the 3′UTR. As another non-limiting example, a miR binding site can be engineered near the 3′ terminus of the 3′UTR and about halfway between the 5′ terminus and 3′ terminus of the 3′UTR. As yet another non-limiting example, a miR binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miR binding sites. The miR binding sites can be complementary to a miR, miR seed sequence, and/or miR sequences flanking the seed sequence.

In one embodiment, an mRNA of the disclosure can be engineered to include more than one miR site expressed in different tissues or different cell types of a subject. In another embodiment, an mRNA of the disclosure can be engineered to include more than one miR site for the same tissue.

In one embodiment, a miR sequence can be incorporated into the loop of a stem loop. In another embodiment, a miR seed sequence can be incorporated in the loop of a stem loop and a miR binding site can be incorporated into the 5′ or 3′ stem of the stem loop.

In one embodiment, the 5′-UTR of a molecule of the disclosure can comprise at least one miR sequence. The miR sequence can be, but is not limited to, a 19 or 22 nucleotide sequence and/or a miR sequence without the seed.

In one embodiment the miR sequence in the 5′UTR can be used to stabilize a molecule of the disclosure described herein.

In another embodiment, a miR sequence in the 5′UTR of an mRNA of the disclosure can be used to decrease the accessibility of the site of translation initiation such as, but not limited to a start codon. See, e.g., Matsuda et al., PLoS One. 2010 11(5):e15057; incorporated herein by reference in its entirety, which used antisense locked nucleic acid (LNA) oligonucleotides and exon-junction complexes (EJCs) around a start codon (−4 to +37 where the A of the AUG codons is +1) in order to decrease the accessibility to the first start codon (AUG). Matsuda showed that altering the sequence around the start codon with an LNA or EJC affected the efficiency, length and structural stability of a molecule. An mRNA of the disclosure can comprise a miR sequence, instead of the LNA or EJC sequence described by Matsuda et al, near the site of translation initiation in order to decrease the accessibility to the site of translation initiation. The site of translation initiation can be prior to, after or within the miR sequence. As a non-limiting example, the site of translation initiation can be located within a miR sequence such as a seed sequence or binding site.

In one embodiment, an mRNA of the disclosure comprises at least one miR sequence in a region of the mRNA that can interact with a RNA binding protein.

Regulation of mRNA Expression

By engineering miR target sequences or binding sites into an mRNA, the mRNA can be targeted for degradation or reduced translation, provided the miR in question is available. This can reduce off-target effects upon delivery of the mRNA. For example, if an mRNA of the disclosure is not intended to be delivered to a tissue or cell but ends up is said tissue or cell, then a miR abundant in the tissue or cell can inhibit the expression of the gene of interest if one or multiple binding sites of the miR are engineered into the 5′UTR and/or 3′UTR of the polynucleotide.

Conversely, miR binding sites can be removed from mRNA sequences in which they naturally occur in order to increase protein expression in specific tissues. For example, a binding site for a specific miR can be removed from an mRNA to improve protein expression in tissues or cells containing the miR.

Regulation of expression in multiple tissues can be accomplished through introduction or removal of one or more miR binding sites, e.g., one or more distinct miR binding sites. The decision whether to remove or insert a miR binding site can be made based on miR expression patterns and/or their profilings in tissues and/or cells in development and/or disease. Identification of miRs, miR binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/1eu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety). In some embodiments, a miR of the disclosure is targeted by incorporating one or more complimentary (e.g., full or partial complementarity) miR binding site(s) into an mRNA encoding a polypeptide of interest wherein incorporation of the one or more miR binding site(s) results in reduced or decreased expression of the mRNA in a specific tissue or cell type (e.g., a cancer cell, an immune cell).

In some embodiments, the therapeutic window and/or differential expression (e.g., tissue-specific expression) of a polypeptide of the disclosure may be altered by incorporation of a miR binding site into an mRNA encoding the polypeptide. In one non-limiting example, an mRNA may include one or more miR binding sites that are bound by miRs that have higher expression in one tissue type as compared to another. In another non-limiting example, an mRNA may include one or more miR binding sites that are bound by miRs that have lower expression in a cancer cell as compared to a non-cancerous cell of the same tissue of origin. When present in a cancer cell that expresses low levels of such a miR, the polypeptide encoded by the mRNA will show increased expression relative to a non-cancerous cell that expresses high levels of the miR. In another non-limiting example, an mRNA comprises one or more miR binding sites complimentary (e.g., full or partial complementarity) to a miR that is abundantly expressed by one immune cell type (e.g., a T cell) but not abundantly expressed by another immune cell type (e.g., a dendritic cell). When present in an immune cell type with low expression of the corresponding miR, higher levels of mRNA expression are achieved than when the mRNA is present in an immune cell type with high expression of the corresponding miR.

In some embodiments, an mRNA of the disclosure can include at least one miR in order to dampen the antigen presentation by antigen presenting cells. The miR can be the complete miR sequence, the miR seed sequence, the miR sequence without the seed, or a combination thereof. As a non-limiting example, a miR incorporated into an mRNA of the disclosure can be specific to the hematopoietic system.

In some embodiments, the mRNA of the disclosure comprises a uracil-modified sequence encoding a polypeptide disclosed herein and a miR binding site disclosed herein. In some embodiments, the uracil-modified sequence encoding a polypeptide comprises at least one chemically modified nucleobase, e.g., 5-methoxyuracil. In some embodiments, at least 95% of a type of nucleobase (e.g., uracil) in a uracil-modified sequence encoding a polypeptide of the disclosure are modified nucleobases. In some embodiments, at least 95% of uracil in a uracil-modified sequence encoding a polypeptide is 5-methoxyuridine. In some embodiments, the mRNA comprising a nucleotide sequence encoding a polypeptide disclosed herein and a miR binding site is formulated with a delivery agent, e.g., a compound having the Formula (I), e.g., Compound II.

In some embodiments, an mRNA of the disclosure (e.g., a RNA, e.g., an mRNA) comprises (i) a sequence-optimized nucleotide sequence (e.g., an ORF) and (ii) one or more miR binding site(s).

In some embodiments, an mRNA of the disclosure is targeted to a tissue or cell by incorporating one or more miR binding site(s) and formulating the mRNA in a lipid nanoparticle comprising an ionizable lipid, including any of the lipids described herein.

### Immune Cell MicroRNA Binding Sites

In some embodiments, an mRNA of the disclosure comprises one or more miR binding site(s) to reduce expression of an encoded polypeptide of interest in a target tissue or cell type. In some embodiments, an mRNA of the disclosure comprising one or more miR binding site(s) complimentary to a miR has reduced expression (e.g., of an encoded polypeptide of interest) when contacted with immune cells that have high abundance or expression of the corresponding miR. In some embodiments, reduced expression of an mRNA comprising one or more miR binding sites is compared to an identical mRNA comprising no miR binding sites (e.g., deletion of the one or more miR binding site(s)). In some embodiments, an mRNA comprising one or more miR binding sites has expression that is reduced by at least 10%, at least 20%, at least 30%, at least 40%, at least 50%, at least 60%, at least 70%, at least 80%, at least 90%, or at least 100% compared to an identical mRNA comprising no miR binding sites (e.g., deletion of the one or more miR binding site(s)). In some embodiments, an mRNA comprising one or more miR binding site(s) has no expression when contacted with immune cells that have high abundance or expression of the corresponding miR.

In some embodiments, an mRNA of the disclosure comprises one or more miR binding site(s) that bind to a miR that is differentially expressed in a type of immune cells comprising B cells. In some embodiments, a miR is differentially expressed by a type of immune cells comprising B cells compared to a type of immune cells comprising T cells, a type of immune cells comprising monocytes, a type of immune cells comprising macrophages, a type of immune cells comprising dendritic cells, and a type of immune cells comprising bone marrow cells. In some embodiments, a miR that is differentially expressed by a type of immune cells comprising B cells is selected from a group identified in Table 1. In some embodiments, an mRNA that comprises one or more miR binding site(s) that bind to a differentially expressed miR selected from a group identified in Table 1 has reduced expression in B cells relative to other immune cell types (e.g., T cells, monocytes, macrophages, DCs, bone marrow cells).

In some embodiments, an mRNA of the disclosure comprises one or more miR binding site(s) that bind to a miR that is differentially expressed in a type of immune cells comprising T cells. In some embodiments, a miR is differentially expressed by a type of immune cells comprising T cells compared to a type of immune cells comprising B cells, a type of immune cells comprising monocytes, a type of immune cells comprising macrophages, a type of immune cells comprising dendritic cells, and a type of immune cells comprising bone marrow cells. In some embodiments, a miR that is differentially expressed by a type of immune cells comprising T cells is selected from a group identified in Table 2. In some embodiments, an mRNA that comprises one or more miR binding site(s) that bind to a differentially expressed miR selected from a group identified in Table 2 has reduced expression in T cells relative to other immune cell types (e.g., B cells, monocytes, macrophages, DCs, bone marrow cells).

In some embodiments, an mRNA of the disclosure comprises one or more miR binding site(s) that bind to a miR that is differentially expressed in a type of immune cells comprising DCs. In some embodiments, a miR is differentially expressed by a type of immune cells comprising DCs compared to a type of immune cells comprising T cells, a type of immune cells comprising monocytes, a type of immune cells comprising macrophages, a type of immune cells comprising B cells, and a type of immune cells comprising bone marrow cells. In some embodiments, a miR that is differentially expressed by a type of immune cells comprising DCs is selected from a group identified in Table 3. In some embodiments, an mRNA that comprises one or more miR binding site(s) that bind to a differentially expressed miR selected from a group identified in Table 3 has reduced expression in DCs relative to other immune cell types (e.g., B cells, T cells, monocytes, macrophages, bone marrow cells).

In some embodiments, an mRNA of the disclosure comprises one or more miR binding site(s) that bind to a miR that is differentially expressed in a type of immune cells comprising monocytes. In some embodiments, a miR is differentially expressed by a type of immune cells comprising monocytes compared to a type of immune cells comprising T cells, a type of immune cells comprising B cells, a type of immune cells comprising macrophages, a type of immune cells comprising dendritic cells, and a type of immune cells comprising bone marrow cells. In some embodiments, a miR that is differentially expressed by a type of immune cells comprising monocytes is selected from a group identified in Table 4. In some embodiments, an mRNA that comprises one or more miR binding site(s) that bind to a differentially expressed miR selected from a group identified in Table 4 has reduced expression in monocytes relative to other immune cell types (e.g., B cells, T cells, DCs, macrophages, bone marrow cells).

In some embodiments, an mRNA of the disclosure comprises one or more miR binding site(s) that bind to a miR that is differentially expressed in a type of immune cells comprising macrophages. In some embodiments, a miR is differentially expressed by a type of immune cells comprising macrophages compared to a type of immune cells comprising T cells, a type of immune cells comprising monocytes, a type of immune cells comprising B cells, a type of immune cells comprising dendritic cells, and a type of immune cells comprising bone marrow cells. In some embodiments, a miR that is differentially expressed by a type of immune cells comprising macrophages is selected from a group identified in Table 5. In some embodiments, an mRNA that comprises one or more miR binding site(s) that bind to a differentially expressed miR selected from a group identified in Table 5 has reduced expression in macrophages relative to other immune cell types (e.g., B cells, T cells, DCs, monocytes, bone marrow cells).

In some embodiments, an mRNA of the disclosure comprises one or more miR binding site(s) that bind to a miR that is differentially expressed in a type of immune cells comprising bone marrow cells. In some embodiments, a miR is differentially expressed by a type of immune cells comprising bone marrow cells compared to a type of immune cells comprising T cells, a type of immune cells comprising monocytes, a type of immune cells comprising macrophages, a type of immune cells comprising dendritic cells, and a type of immune cells comprising B cells. In some embodiments, a miR that is differentially expressed by a type of immune cells comprising bone marrow cells is selected from a group identified in Table 6. In some embodiments, an mRNA that comprises one or more miR binding site(s) that bind to a differentially expressed miR selected from a group identified in Table 6 has reduced expression in bone marrow cells relative to other immune cell types (e.g., B cells, T cells, DCs, monocytes, macrophages).

In some embodiments, an mRNA of the disclosure comprises one or more miR binding site(s) that binds to a miR that is abundantly expressed by a first type of immune cells, a second type of immune cells, a third type of immune cells, a fourth type of immune cells, and a fifth type of immune cells. In some embodiment, a first type of immune cells comprises T cells, a second type of immune cells comprises B cells, a third type of immune cells comprises monocytes, a fourth type of immune cells comprises macrophages, and a fifth type of immune cells comprises DCs. In some embodiments, a miR that is abundantly expressed by types of immune cells that comprise T cells, B cells, monocytes, macrophages and DCs is miR-142-3p. In some embodiments, an mRNA of the disclosure comprises one or more miR binding site(s) that bind to miR-142-3p. In some embodiments, an mRNA comprising one or more miR binding site(s) that bind to miR-142-3p has reduced expression in types of immune cells (e.g., T cells, B cells, monocytes, macrophages, DCs) that abundantly express miR-142-3p.

In some embodiments, an mRNA of the disclosure comprises one or more miR binding site(s) that binds to a miR that is abundantly expressed by a first type of immune cells, a second type of immune cells, a third type of immune cells, and a fourth type of immune cells, but not abundantly expressed by a fifth type of immune cells. In some embodiment, a first type of immune cells comprises T cells, a second type of immune cells comprises B cells, a third type of immune cells comprises monocytes, a fourth type of immune cells comprises macrophages, and a fifth type of immune cells comprises DCs. In some embodiments, a miR that is abundantly by immune cells comprising T cells, B cells, monocytes, and macrophages, but not abundantly expressed by DCs is miR-150-3p or miR-29b-3p. In some embodiments, an mRNA comprising one or more miR binding site(s) that bind to miR-150-3p has reduced expression in types of immune cells (e.g., T cells, B cells, monocytes, macrophages) that abundantly express miR-150-3p. In some embodiments, an mRNA comprising one or more miR binding site(s) that bind to miR-29b-3p has reduced expression in types of immune cells (e.g., T cells, B cells, monocytes, macrophages) that abundantly express miR-29b-3p.

In some embodiments, an mRNA of the disclosure comprises one or more miR binding site(s) that binds to a miR that is abundantly expressed by a first type of immune cells, a second type of immune cells, a third type of immune cells, and a fourth type of immune cells, but not abundantly expressed by a fifth type of immune cells. In some embodiment, a first type of immune cells comprises B cells, a second type of immune cells comprises monocytes, a third type of immune cells comprises macrophages, a fourth type of immune cells comprises DCs, and a fifth type of immune cells comprises T cells. In some embodiments, a miR that is abundantly expressed by B cells, monocytes, macrophages and DCs, but not abundantly expressed by T cells is miR-16a-5p. In some embodiments, an mRNA comprising one or more miR binding site(s) that bind to miR-16a-5p has reduced expression in types of immune cells (e.g., B cells, monocytes, macrophages, DCs) that abundantly express miR-29b-3p.

In some embodiments, an mRNA of the disclosure comprises one or more miR binding site(s) that binds to a miR that is abundantly expressed by a first type of immune cells, a second type of immune cells, and a third type of immune cells, but not abundantly expressed by a fourth type of immune cells or a fifth type of immune cells.

In some embodiments, an mRNA of the disclosure comprises one or more miR binding site(s) that binds to a miR that is abundantly expressed by a first type of immune cells and a second type of immune cells, but not abundantly expressed by a third type of immune cells, a fourth type of immune cells, or a fifth type of immune cells. In some embodiment, a first type of immune cells comprises monocytes, a second type of immune cells comprises macrophages, a third type of immune cells comprises B cells, a fourth type of immune cells comprises T cells, and a fifth type of immune cells comprises DCs. In some embodiments, miRs that are abundantly expressed by monocytes and macrophages, but not abundantly expressed by B cells, T cells, or DCs are miR-4454 and/or miR-7975. In some embodiments, an mRNA comprising one or more miR binding site(s) that bind to miR-4454 and/or miR-7975 has reduced expression in types of immune cells (e.g., monocytes, macrophages) that abundantly express miR-4454 and/or miR-7975.

In some embodiments, an mRNA of the disclosure comprises one or more miR binding site(s) that binds to a miR that is abundantly expressed by a first type of immune cells and a second type of immune cells, but not abundantly expressed by a third type of immune cells, a fourth type of immune cells, or a fifth type of immune cells. In some embodiment, a first type of immune cells comprises B cells, a second type of immune cells comprises T cells, a third type of immune cells comprises monocytes, a fourth type of immune cells comprises macrophages, and a fifth type of immune cells comprises DCs. In some embodiments, a miR that is abundantly expressed by B cells and T cells, but not abundantly expressed by monocytes, macrophages, or DCs is let7g-5p. In some embodiments, an mRNA comprising one or more miR binding site(s) that bind to let7g-5p has reduced expression in types of immune cells (e.g., B cells, T cells) that abundantly express let7g-5p.

In some embodiments, an mRNA of the disclosure comprises one or more miR binding site(s) that bind to a miR that is abundantly expressed by a first type of immune cells, but not abundantly expressed by a second type of immune cells, a third type of immune cells, a fourth type of immune cells, or a fifth type of immune cells. In some embodiments, a first type of immune cells comprises T cells, a second type of immune cells comprises B cells, a third type of immune cells comprises macrophages, a fourth type of immune cells comprises monocytes, and a fifth type of immune cells comprises DCs. In some embodiments, a miR that is abundantly expressed by T cells, but not abundantly expressed by B cells, macrophages, monocytes or DCs is miR-342-3p. In some embodiments, an mRNA comprising one or more miR binding site(s) that bind to miR-342-3p has reduced expression in immune cells (e.g., T cells) that abundantly express miR-342-3p.

In some embodiments, an mRNA of the disclosure comprises one or more miR binding site(s) that bind to a miR that is abundantly expressed by a first type of immune cells, but not abundantly expressed by a second type of immune cells, a third type of immune cells, a fourth type of immune cells, or a fifth type of immune cells. In some embodiment, a first type of immune cells comprise DCs, a second type of immune cells comprises T cells, a third type of immune cells comprises B cells, a fourth type of immune cells comprises macrophages, and a fifth type of immune cells comprises monocytes. In some embodiments, a miR that is abundantly expressed by DCs, but not abundantly expressed by B cells, T cells, monocytes or macrophages is selected from a group consisting of: miR-21-5p, miR-223-3p, or let7a-5p. In some embodiments, an mRNA comprising one or more miR binding site(s) that bind to miR-21-5p has reduced expression in immune cells (e.g., DCs) that abundantly express miR-21-5p. In some embodiments, an mRNA comprising one or more miR binding site(s) that bind to miR-223-3p has reduced expression in immune cells (e.g., DCs) that abundantly express miR-223-3p. In some embodiments, an mRNA comprising one or more miR binding site(s) that bind to let7a-5p has reduced expression in immune cells (e.g., DCs) that abundantly express let7a-5p.

In some embodiments, an mRNA of the disclosure comprising one or more miR binding site(s) has reduced expression in a first type of immune cells, a second type of immune cells, a third type of immune cells, a fourth type of immune cells, a fifth type of immune cells, and a sixth type of immune cells. In some embodiments, a first type of immune cells is comprised of T cells, a second type of immune cells is comprised of DCs, a third type of immune cells is comprised of neutrophils, a fourth type of immune cells is comprised of NK cells, a fifth type of immune cells is comprised of monocytes, and a sixth type of immune cells is comprised of macrophages. In some embodiments, the one or more miR binding site(s) that reduces expression of an mRNA in a first type of immune cells, a second type of immune cells, a third type of immune cells, a fourth type of immune cells, a fifth type of immune cells, and a sixth type of immune cells binds to miR-142-3p. In some embodiments, the one or more miR binding site(s) that reduces expression of an mRNA in T cells, DCs, neutrophils, NK ells, monocytes, and macrophages binds to miR-142-3p.

In some embodiments, an mRNA of the disclosure comprising one or more miR binding site(s) has reduced expression in a first type of immune cells, a second type of immune cells, a third type of immune cells, a fourth type of immune cells, a fifth type of immune cells, but does not have reduced expression in a sixth type of immune cells. In some embodiments, a first type of immune cells is comprised of T cells, a second type of immune cells is comprised of macrophages, a third type of immune cells is comprised of neutrophils, a fourth type of immune cells is comprised of NK cells, a fifth type of immune cells is comprised of monocytes, and a sixth type of immune cells is comprised of DCs. In some embodiments, the one or more miR binding site(s) that reduces expression of an mRNA in a first type of immune cells, a second type of immune cells, a third type of immune cells, a fourth type of immune cells, a fifth type of immune cells, but does not reduce expression in a sixth type of immune cells, binds to miR-23a-3p. In some embodiments, the one or more miR binding site(s) that reduces expression of an mRNA in T cells, neutrophils, NK ells, monocytes, and macrophages but does not reduce expression in DCs, binds to miR-23a-3p.

In some embodiments, an mRNA of the disclosure comprising one or more miR binding site(s) has reduced expression in a first type of immune cells, a second type of immune cells, a third type of immune cells and a fourth type of immune cells, but does not have reduced expression in a fifth type of immune cells or a sixth type of immune cells. In some embodiments, a first type of immune cells is comprised of DCs, a second type of immune cells is comprised of macrophages, a third type of immune cells is comprised of NK cells, a fourth type of immune cells is comprised of monocytes, a fifth type of immune cells is comprised of T cells, and a sixth type of immune cells is comprised of neutrophils. In some embodiments, the one or more miR binding site(s) that reduces expression of an mRNA in a first type of immune cells, a second type of immune cells, a third type of immune cells, and a fourth type of immune cells, but does not reduce expression in a fifth type of immune cells or a sixth type of immune cells binds to miR-223-3p. In some embodiments, the one or more miR binding site(s) that reduces expression of an mRNA in DCs, macrophages, NK cells, and monocytes, but does not reduce expression in T cells or neutrophils binds to miR-223-3p.

In some embodiments, an mRNA of the disclosure comprising one or more miR binding site(s) has reduced expression in a first type of immune cells, a second type of immune cells, and a third type of immune cells, but does not have reduced expression in a fourth type of immune cells, a fifth type of immune cells or a sixth type of immune cells.

In some embodiments, an mRNA of the disclosure comprising one or more miR binding site(s) that reduced expression in a first type of immune cells and a second type of immune cells, but does not have reduced expression in a third type of immune cells, a fourth type of immune cells, a fifth type of immune cells or a sixth type of immune cells. In some embodiments, a first type of immune cells is comprised of T cells, a second type of immune cells is comprised of neutrophils, a third type of immune cells is comprised of DCs, a fourth type of immune cells is comprised of macrophages, a fifth type of immune cells is comprised of NK cells, and a sixth type of immune cells is comprised of monocytes. In some embodiments, the one or more miR binding site(s) that reduces expression of an mRNA in a first type of immune cells and a second type of immune cells, but does not reduce expression in a third type of immune cells, a fourth type of immune cells, a fifth type of immune cells or a sixth type of immune cells binds to miR-150-5p. In some embodiments, the one or more miR binding site(s) that reduces expression of an mRNA in T cells and neutrophils, but does not reduce expression in DCs, macrophages, NK cells, or monocytes binds to miR-150-5p.

In some embodiments, an mRNA of the disclosure comprising one or more miR binding site(s) has reduced expression in a first type of immune cells and a second type of immune cells, but does not have reduced expression in a third type of immune cells, a fourth type of immune cells, a fifth type of immune cells or a sixth type of immune cells. In some embodiments, a first type of immune cells is comprised of T cells, a second type of immune cells is comprised of NK cells, a third type of immune cells is comprised of DCs, a fourth type of immune cells is comprised of neutrophils, a fifth type of immune cells is comprised of macrophages, and a sixth type of immune cells is comprised of monocytes. In some embodiments, the one or more miR binding site(s) that reduces expression of an mRNA in a first type of immune cells and a second type of immune cells, but does not reduce expression in a third type of immune cells, a fourth type of immune cells, a fifth type of immune cells or a sixth type of immune cells binds to miR-146-5p. In some embodiments, the one or more miR binding site(s) that reduces expression of an mRNA in T cells or NK cells, but does not reduce expression in DCs, neutrophils, macrophages, and monocytes binds to miR-146-5p.

In some embodiments, an mRNA of the disclosure comprising one or more miR binding site(s) has reduced expression in a first type of immune cells, but does not have reduced expression in a second type of immune cells, a third type of immune cells, a fourth type of immune cells, a fifth type of immune cells or a sixth type of immune cells. In some embodiments, a first type of immune cells is comprised of T cells, a second type of immune cells is comprised of NK cells, a third type of immune cells is comprised of DCs, a fourth type of immune cells is comprised of neutrophils, a fifth type of immune cells is comprised of macrophages, and a sixth type of immune cells is comprised of monocytes. In some embodiments, the one or more miR binding site(s) that reduces expression of an mRNA in a first type of immune cells, but does not reduce expression in a second type of immune cells, a third type of immune cells, a fourth type of immune cells, a fifth type of immune cells or a sixth type of immune cells binds to miR-21-5p. In some embodiments, the one or more miR binding site(s) that reduces expression of an mRNA in T cells, but does not reduce expression in NK cells, DCs, neutrophils, macrophages, or monocytes binds to miR-21-5p.

In some embodiments, an mRNA of the disclosure comprising one or more miR binding site(s) has reduced expression in a first type of immune cells, but does not have reduced expression in a second type of immune cells, a third type of immune cells, a fourth type of immune cells, a fifth type of immune cells or a sixth type of immune cells. In some embodiments, a first type of immune cells is comprised of neutrophils, a second type of immune cells is comprised of T cells, a third type of immune cells is comprised of NK cells, a fourth type of immune cells is comprised of DCs, a fifth type of immune cells is comprised of macrophages, and a sixth type of immune cells is comprised of monocytes. In some embodiments, the one or more miR binding site(s) that reduces expression of an mRNA in a first type of immune cells, but does not reduce expression in a second type of immune cells, a third type of immune cells, a fourth type of immune cells, a fifth type of immune cells or a sixth type of immune cells binds to a mature miR derived from miR-143. In some embodiments, the one or more miR binding site(s) that reduces expression of an mRNA in neutrophils, but does not reduce expression in T cells, NK cells, DCs, macrophages, or monocytes binds to a mature miR derived from miR-143.

In some embodiments, an mRNA of the disclosure comprising one or more miR binding site(s) has reduced expression in T cells. In some embodiments, an mRNA that has reduced expression in T cells comprises one or more miR binding site(s) that bind to miR-146-5p and one or more miR binding site(s) that bind to at least one additional miR selected from a group consisting of: miR-23a-3p, miR-142-3p, miR-150-5p, and/or miR-21-5p. In some embodiments, an mRNA that has reduced expression in T cells comprises one or more miR binding site(s) that bind to miR-23a-3p and one or more miR binding site(s) that bind to at least one additional miR selected from a group consisting of: miR-146-5p, miR-142-3p, miR-150-5p, and/or miR-21-5p. In some embodiments, an mRNA that has reduced expression in T cells comprises one or more miR binding site(s) that bind to miR-142-3p and one or more miR binding site(s) that bind to at least one additional miR selected from a group consisting of: miR-146-5p, miR-23a-3p, miR-150-5p, and/or miR-21-5p. In some embodiments, an mRNA that has reduced expression in T cells comprises one or more miR binding site(s) that bind to miR-150-5p and one or more miR binding site(s) that bind to at least one additional miR selected from a group consisting of: miR-146-5p, miR-23a-3p, miR-142-3p, and/or miR-21-5p. In some embodiments, an mRNA that has reduced expression in T cells comprises one or more miR binding site(s) that bind to miR-21-5p and one or more miR binding site(s) that bind to at least one additional miR selected from a group consisting of: miR-146-5p, miR-23a-3p, miR-142-3p, and/or miR-150-5p.

In some embodiments, an mRNA of the disclosure comprising one or more miR binding site(s) has reduced expression in T cells. In some embodiments, an mRNA that has reduced expression in T cells comprises one or more miR binding site(s) that bind to miR-146-5p, one or more miR binding site(s) that bind to miR-23a-3p, one or more miR binding site(s) that bind to miR-142-3p, one or more miR binding site(s) that bind to miR-150-5p, and one or more miR binding site(s) that bind to miR-21-5p.

In some embodiments, an mRNA of the disclosure comprising one or more miR binding site(s) has reduced expression in DCs. In some embodiments, an mRNA that has reduced expression in DCs comprises one or more miR binding site(s) that bind to miR-142-3p and one or more miR binding site(s) that bind to miR-223-3p.

In some embodiments, an mRNA of the disclosure comprising one or more miR binding site(s) has reduced expression in neutrophils. In some embodiments, an mRNA that has reduced expression in neutrophils comprises one or more miR binding site(s) that bind to miR-23a-3p and one or more miR binding site(s) that bind to at least one additional miR selected from a group consisting of: miR-142-3p, miR-150-5p, and/or miR-143-XX. In some embodiments, an mRNA that has reduced expression in neutrophils comprises one or more miR binding site(s) that bind to miR-142-3p and one or more miR binding site(s) that bind to at least one additional miR selected from a group consisting of: miR-23a-3p, miR-150-5p, and/or miR-143-XX. In some embodiments, an mRNA that has reduced expression in neutrophils comprises one or more miR binding site(s) that bind to miR-150-5p and one or more miR binding site(s) that bind to at least one additional miR selected from a group consisting of: miR-23a-3p, miR-142-3p, and/or a mature miR derived from miR-143. In some embodiments, an mRNA that has reduced expression in neutrophils comprises one or more miR binding site(s) that bind to miR-143-XX and one or more miR binding site(s) that bind to at least one additional miR selected from a group consisting of: miR-23a-3p, miR-142-3p, and/or miR-150-5p.

In some embodiments, an mRNA of the disclosure comprising one or more miR binding site(s) has reduced expression in neutrophils. In some embodiments, an mRNA that has reduced expression in neutrophils comprises one or more miR binding site(s) that bind to miR-23a-3p, one or more miR binding site(s) that bind to miR-142-3p, one or more miR binding site(s) that bind to miR-150-5p, and one or more miR binding site(s) that bind to a mature miR derived from miR-143.

In some embodiments, an mRNA of the disclosure comprising one or more miR binding site(s) has reduced expression in NK cells. In some embodiments, an mRNA that has reduced expression in NK cells comprises one or more miR binding site(s) that bind to miR-146-5p and one or more miR binding site(s) that bind to at least one additional miR selected from a group consisting of: miR-23a-3p, miR-142-3p, and/or miR-223-3p. In some embodiments, an mRNA that has reduced expression in NK cells comprises one or more miR binding site(s) that bind to miR-23a-3p and one or more miR binding site(s) that bind to at least one additional miR selected from a group consisting of: miR-146-5p, miR-142-3p, and/or miR-223-3p. In some embodiments, an mRNA that has reduced expression in NK cells comprises one or more miR binding site(s) that bind to miR-142-3p and one or more miR binding site(s) that bind to at least one additional miR selected from a group consisting of: miR-146-5p, miR-23a-3p, and/or miR-223-3p. In some embodiments, an mRNA that has reduced expression in NK cells comprises one or more miR binding site(s) that bind to miR-223-3p and one or more miR binding site(s) that bind to at least one additional miR selected from a group consisting of: miR-146-5p, miR-23a-3p, and/or miR-142-3p.

In some embodiments, an mRNA of the disclosure comprising one or more miR binding site(s) has reduced expression in NK cells. In some embodiments, an mRNA that has reduced expression in NK cells comprises one or more miR binding site(s) that bind to miR-146-5p, one or more miR binding site(s) that bind to miR-23a-3p, one or more miR binding site(s) that bind to miR-142-3p, and one or more miR binding site(s) that bind to miR-223-3p.

In some embodiments, an mRNA of the disclosure comprising one or more miR binding site(s) has reduced expression in macrophages. In some embodiments, an mRNA that has reduced expression in macrophages comprises one or more miR binding site(s) that bind to miR-23a-3p and one or more miR binding site(s) that bind to at least one additional miR selected from a group consisting of: miR-142-3p and/or miR-223-3p. In some embodiments, an mRNA that has reduced expression in macrophages comprises one or more miR binding site(s) that bind to miR-142-3p and one or more miR binding site(s) that bind to at least one additional miR selected from a group consisting of: miR-23a-3p and/or miR-223-3p. In some embodiments, an mRNA that has reduced expression in macrophages comprises one or more miR binding site(s) that bind to miR-223-3p and one or more miR binding site(s) that bind to at least one additional miR selected from a group consisting of: miR-23a-3p and/or miR-142-3p.

In some embodiments, an mRNA of the disclosure comprising one or more miR binding site(s) has reduced expression in macrophages. In some embodiments, an mRNA that has reduced expression in macrophages comprises one or more miR binding site(s) that bind to miR-23a-3p, one or more miR binding site(s) that bind to miR-23a-3p, one or more miR binding site(s) that bind to miR-142-3p, and one or more miR binding site(s) that bind to miR-223-3p.

In some embodiments, an mRNA of the disclosure comprising one or more miR binding site(s) has reduced expression in monocytes. In some embodiments, an mRNA that has reduced expression in monocytes comprises one or more miR binding site(s) that bind to miR-23a-3p and one or more miR binding site(s) that bind to at least one additional miR selected from a group consisting of: miR-142-3p and/or miR-223-3p. In some embodiments, an mRNA that has reduced expression in macrophages comprises one or more miR binding site(s) that bind to miR-142-3p and one or more miR binding site(s) that bind to at least one additional miR selected from a group consisting of: miR-23a-3p and/or miR-223-3p. In some embodiments, an mRNA that has reduced expression in macrophages comprises one or more miR binding site(s) that bind to miR-223-3p and one or more miR binding site(s) that bind to at least one additional miR selected from a group consisting of: miR-23a-3p and/or miR-142-3p.

In some embodiments, an mRNA of the disclosure comprising one or more miR binding site(s) has reduced expression in monocytes. In some embodiments, an mRNA that has reduced expression in macrophages comprises one or more miR binding site(s) that bind to miR-23a-3p, one or more miR binding site(s) that bind to miR-23a-3p, one or more miR binding site(s) that bind to miR-142-3p, and one or more miR binding site(s) that bind to miR-223-3p.

### Cancer Related MicroRNA Binding Sites

In some embodiments, an mRNA of the disclosure comprises one or more miR binding site(s) to reduce expression of an encoded polypeptide of interest in a target tissue or cell type. In some embodiments, a target cell type is a transformed immune cell (e.g., a cancerous immune cell). In some embodiments, a target cell type is a healthy immune cell (e.g., a non-cancerous immune cells). In some embodiments, an mRNA of the disclosure comprising one or more miR binding site(s) complimentary to a miR has reduced expression (e.g., of an encoded polypeptide of interest) when contacted with transformed immune cells (e.g., cancerous immune cells) that have high abundance or expression of the corresponding miR. In some embodiments, an mRNA of the disclosure comprising one or more miR binding site(s) complimentary to a miR has reduced expression (e.g., of an encoded polypeptide of interest) when contacted with healthy immune cells (e.g., non-cancerous immune cells) that have high abundance or expression of the corresponding miR.

In some embodiments, reduced expression of an mRNA comprising one or more miR binding sites is compared to an identical mRNA comprising no miR binding sites (e.g., deletion of the one or more miR binding site(s)). In some embodiments, an mRNA comprising one or more miR binding sites has expression that is reduced by at least 10%, at least 20%, at least 30%, at least 40%, at least 50%, at least 60%, at least 70%, at least 80%, at least 90%, or at least 100% compared to an identical mRNA comprising no miR binding sites (e.g., deletion of the one or more miR binding site(s)). In some embodiments, an mRNA comprising one or more miR binding site(s) has no expression when contacted with transformed immune cells (e.g., cancerous immune cells) that have high abundance or expression of the corresponding miR. In some embodiments, an mRNA comprising one or more miR binding site(s) has no expression when contacted with healthy immune cells (e.g., non-cancerous immune cells) that have high abundance or expression of the corresponding miR.

In some embodiments, an mRNA of the disclosure comprises one or more miR binding site(s) that binds to a miR expressed by a type of cancer cells. In some embodiments, a type of cancer cells are AML cells. In some embodiments, a miR that is highly or abundantly expressed by a type of cancer cells comprising AML cells is selected from a group shown in Table 7. In some embodiments, an mRNA of the disclosure comprises one or more miR binding site(s) that binds to a miR selected from a group show in Table 7.

In some embodiments, an mRNA of the disclosure comprises one or more miR binding site(s) that binds to a miR that is differentially expressed in a type of cancer cells (e.g., AML cells). In some embodiments, a miR is differentially expressed in a type of cancer cells (e.g., AML cells) compared to a plurality of non-cancerous cells. In some embodiments, non-cancerous cells comprise a type of immune cell that includes any one or more of bone marrow cells, B cells, T cells, macrophages, or monocytes. In some embodiments, a miR that is differentially expressed by AML cells compared to a plurality of non-cancerous cells is selected from a group consisting of: miR-18a-5p, miR-1246, or miR-126-3p. In some embodiments, an mRNA of the disclosure comprising one or more miR binding site(s) that binds to miR-18a-5p has reduced expression in AML cells compared to a plurality of non-cancerous immune cells. In some embodiments, an mRNA of the disclosure comprising one or more miR binding site(s) that binds to miR-1246 has reduced expression in AML cells compared to a plurality of non-cancerous immune cells. In some embodiments, an mRNA of the disclosure comprising one or more miR binding site(s) that binds to miR-126-3p has reduced expression in AML cells compared to a plurality of non-cancerous immune cells.

In some embodiments, an mRNA of the disclosure comprises one or more miR binding site(s) that binds to a miR that is differentially expressed in a type of non-cancerous immune cells. In some embodiments, a type of non-cancerous immune cells comprises bone marrow cells, B cells, macrophages, DCs, T cells, monocytes or any combination thereof. In some embodiments, a miR is differentially expressed in one or more types of non-cancerous immune cells compared to one or more types of cancerous cells (e.g., AML cells). In some embodiments, a miR that is differentially expressed by non-cancerous immune cells compared to AML cells is selected from a group consisting of: miR-150-5p, miR-146-5p, miR-4286, miR-579-3p, miR-4516, miR-146a-5p, miR-664b-3p, miR-342-3p, miR-1915-3p, or miR-26b-5p. In some embodiments, an mRNA of the disclosure comprising one or more miR binding site(s) that binds to any one or more of miR-150-5p, miR-146-5p, miR-4286, miR-579-3p, miR-4516, miR-146a-5p, miR-664b-3p, miR-342-3p, miR-1915-3p, or miR-26b-5p has reduced expression in non-cancerous cells compared to AML cells. In some embodiments, an mRNA of the disclosure comprising one or more miR binding site(s) that binds to miR-150-5p has reduced expression in non-cancerous cells compared to AML cells.

Some miRs are abundant in normal tissue but are present in much lower levels in cancer or tumor tissue (e.g., AML cells) (e.g., miR-150-5p, miR-146b-5p, miR-4286, miR-579-3b, miR-4516, miR-146a-5p, miR-664b-3p, miR-342-3p, miR-342-5p, miR-1915-3p, and/or miR-26b-5p). Thus, engineering miR miR binding site(s) into an mRNA (e.g., in a 3′UTR region or another region) can effectively target the mRNA for degradation or reduce translation in normal tissue (where the miR is abundant) while providing high levels of translation in the cancer or tumor tissue (where the miR is present in much lower levels). This provides an approach for differential tumor-targeting relative to normal cells.

In some embodiments, at least one miR-binding site for a miR expressed in lower levels in AML cells, (e.g., miR-150-5p, miR-146b-5p, miR 4286, miR-579-3b, miR-4516, miR-146a-5p, miR-664b-3p, miR-342-3p, miR-342-5p, miR-1915-3p, or miR-26b-5p) is engineered into an mRNA encoding a cytotoxic protein, e.g., an apoptotic or suicide protein, or a protein that destroys a tumor cell growth factor,

Some microRNAs are abundant in AML cells but are present in much lower levels in normal immune cells (e.g., miR-18a-5p, miR-1246, and miR-126-3p). Thus, engineering miR binding site(s) into an mRNA (e.g., in a 3′UTR region or other region) can effectively target the mRNA for degradation or reduce translation in cancer or tumor tissue (e.g., AML cells, where the miR is abundant) while providing high levels of translation in normal cells (where the miR is present in much lower levels). This provides an approach for differential targeting of normal immune cells relative to tumor cells. In some embodiments, at least one miR-binding site for a miR expressed in lower levels in normal immune cells relative to AML cells, (e.g., miR-18a-5p, miR-1246 or miR 126-3p), is engineered into an mRNA encoding a chimeric antigen receptor (CAR-T) or T cell receptor (TCR), constitutively active cytokine receptor, or anti-tumor effector molecules such as granzyme or perforin.

TABLE 8
miRs and miR-binding sites differentially
expressed in AML cells

| SEQ ID | | |
|---|---|---|
| NO. | Description | Sequence |
| 1 | miR-150-5p | UCUCCCAACCCUUGUACCAGUG |
| 2 | miR-150-5p | CACTGGTACAAGGGTTGGGAGA |
| | binding site | |
| 3 | miR-146b-5p | UGAGAACUGAAUUCCAUAGGCUG |
| 4 | miR-146b-5p | CAGCCTATGGAATTCAGTTCTCA |
| | binding site | |
| 5 | miR 4286 | ACCCCACUCCUGGUACC |
| 6 | miR-4286 | GGTACCAGGAGTGGGGT |
| | binding site | |
| 7 | miR-579-3b | UUCAUUUGGUAUAAACCGCGAUU |
| 8 | miR-579-3b | AATCGCGGTTTATACCAAATGAA |
| | binding site | |
| 9 | miR-4516 | GGGAGAAGGGUCGGGGC |
| 10 | miR-4516 | GCCCCGACCCTTCTCCC |
| | binding site | |
| 11 | miR-146a-5p | UGAGAACUGAAUUCCAUGGGUU |
| 12 | miR-146a-5p | AACCCATGGAATTCAGTTCTCA |
| | binding site | |
| 13 | miR-664b-3p | UUCAUUUGCCUCCCAGCCUACA |
| 14 | miR-664b-3p | TGTAGGCTGGGAGGCAAATGAA |
| | binding site | |
| 15 | miR-342-3p | UCUCACACAGAAAUCGCACCCGU |
| 16 | miR-342-3p | ACGGGTGCGATTTCTGTGTGAGA |
| | binding site | |
| 17 | miR-342-5p | AGGGGUGCUAUCUGUGAUUGA |
| 18 | miR-342-5p | TCAATCACAGATAGCACCCCT |
| | binding site | |
| 19 | miR-1915-3p | CCCCAGGGCGACGCGGCGGG |
| 20 | miR-1915-3p | CCCGCCGCGTCGCCCTGGGG |
| | binding site | |
| 21 | miR-26b-5p | UUCAAGUAAUUCAGGAUAGGU |
| 22 | miR-26b-5p | ACCTATCCTGAATTACTTGAA |
| | binding site | |
| 23 | miR-18a-5p | UAAGGUGCAUCUAGUGCAGAUAG |
| 24 | miR-18a-5p | CTATCTGCACTAGATGCACCTTA |
| | binding site | |
| 25 | miR-1246 | AAUGGAUUUUUGGAGCAGG |
| 26 | miR-1246 | CCTGCTCCAAAAATCCATT |
| | binding site | |
| 27 | miR 126-3p | UCGUACCGUGAGUAAUAAUGCG |
| 28 | miR 126-3p | CGCATTATTACTCACGGTACGA |
| | binding site | |

### Immune Cell Subset MicroRNA Binding Sites

In some embodiments, an mRNA of the disclosure comprises one or more miR binding site(s) to reduce expression of an encoded polypeptide of interest in a target tissue or cell type. In some embodiments, a target cell is a type of immune cell (e.g., naïve T cells, effector T cells, regulatory T cells, activated T cells). In some embodiments, a target cell is a specific cell subset (e.g., naïve T cells, effector T cells, regulatory T cells). In some embodiments, a target cell is a specific cell state (e.g., activated T cells, resting T cells). In some embodiments, an mRNA of the disclosure comprising one or more miR binding site(s) complimentary to a miR has reduced expression (e.g., of an encoded polypeptide of interest) when contacted with target cells of a specific subset (e.g., naïve T cells, effector T cells, regulatory T cells) that have high abundance or expression of the corresponding miR. In some embodiments, an mRNA of the disclosure comprising one or more miR binding site(s) complimentary to a miR has reduced expression (e.g., of an encoded polypeptide of interest) when contacted with target cells of a specific cell state (e.g., activated T cells, resting T cells) that have high abundance or expression of the corresponding miR.

In some embodiments, reduced expression of an mRNA comprising one or more miR binding sites is compared to an identical mRNA comprising no miR binding sites (e.g., deletion of the one or more miR binding site(s)). In some embodiments, an mRNA comprising one or more miR binding sites has expression that is reduced by at least 10%, at least 20%, at least 30%, at least 40%, at least 50%, at least 60%, at least 70%, at least 80%, at least 90%, or at least 100% compared to an identical mRNA comprising no miR binding sites (e.g., deletion of the one or more miR binding site(s)). In some embodiments, an mRNA comprising one or more miR binding site(s) has no expression when contacted with target cells of a specific subset (e.g., naïve T cells, effector T cells, regulatory T cells) that have high abundance or expression of the corresponding miR. In some embodiments, an mRNA comprising one or more miR binding site(s) has no expression when contacted with target cells of a specific cell state (e.g., activated T cells, resting T cells) that have high abundance or expression of the corresponding miR.

In some embodiments, an mRNA of the disclosure comprises one or more miR binding site(s) that binds at least one miR that is highly or abundantly expressed by a specific subset of T cells comprising naïve T cells, wherein a miR is any one selected from a group consisting of: miR-150-5p, miR-142-3p, miR-342-3p, let-7g-5p, or miR-29b-3p. In some embodiments, an mRNA of the disclosure comprising one or more miR binding site(s) to one or more of miR-150-5p, miR-142-3p, miR-342-3p, let-7g-5p, or miR-29b-3p has reduced expression in naïve T cells. In some embodiments, an mRNA of the disclosure comprises one or more miR binding site(s) that binds at least one miR that is highly or abundantly expressed by a specific subset of T cells comprising effector T cells, wherein a miR is any one selected from a group consisting of: miR-150-5p, miR-142-3p, miR-29b-3p, miR-342-3p, let-7g-5p. In some embodiments, an mRNA of the disclosure comprising one or more miR binding site(s) to one or more of miR-150-5p, miR-142-3p, miR-29b-3p, miR-342-3p, let-7g-5p has reduced expression in effector T cells. In some embodiments, an mRNA of the disclosure comprises one or more miR binding site(s) that binds at least one miR that is highly or abundantly expressed by a specific subset of T cells comprising regulatory T cells, wherein a miR is any one selected from a group consisting of: miR-150-5p, miR-142-3p, miR-29b-3p, miR-146a-5p, or miR-223-3p. In some embodiments, an mRNA of the disclosure comprising one or more miR binding site(s) to one or more of miR-150-5p, miR-142-3p, miR-29b-3p, miR-146a-5p, or miR-223-3p has reduced expression in regulatory T cells.

In some embodiments, an mRNA of the disclosure comprises one or more miR binding site(s) that binds at least one miR that is highly or abundantly expressed by a specific state of T cells comprising resting cells. In some embodiments, an mRNA of the disclosure comprises one or more miR binding site(s) that binds at least one miR that is highly or abundantly expressed by a specific state of T cells comprising resting CD3+ T cells, wherein a miR is any one selected from a group consisting of: miR-150-5p, miR-142-3p, miR-29b-3p, let-7g-5p, or miR-342-3p. In some embodiments, an mRNA of the disclosure comprising one or more miR binding site(s) to one or more of miR-150-5p, miR-142-3p, miR-29b-3p, let-7g-5p, or miR-342-3p has reduced expression in resting CD3+ T cells. In some embodiments, an mRNA of the disclosure comprises one or more miR binding site(s) that binds at least one miR that is highly or abundantly expressed by a specific state of T cells comprising resting CD3+CD4+ T cells, wherein a miR is any one selected from a group consisting of: miR-150-5p, miR-142-3p, miR-29b-3p, let-7g-5p, or miR-342-3p. In some embodiments, an mRNA of the disclosure comprising one or more miR binding site(s) to one or more of miR-150-5p, miR-142-3p, miR-29b-3p, let-7g-5p, or miR-342-3p has reduced expression in resting CD3+CD4+ T cells. In some embodiments, an mRNA of the disclosure comprises one or more miR binding site(s) that binds at least one miR that is highly or abundantly expressed by a specific state of T cells comprising resting CD3+CD8+ T cells, wherein a miR is any one selected from a group consisting of: miR-150-5p, miR-142-3p, miR-29b-3p, let-7g-5p, miR-342-3p. In some embodiments, an mRNA of the disclosure comprising one or more miR binding site(s) to one or more of miR-150-5p, miR-142-3p, miR-29b-3p, let-7g-5p, miR-342-3p has reduced expression in resting CD3+CD8+ T cells.

In some embodiments, an mRNA of the disclosure comprises one or more miR binding site(s) that binds at least one miR that is highly or abundantly expressed by a specific state of T cells comprising activated cells. In some embodiments, an mRNA of the disclosure comprises one or more miR binding site(s) that binds at least one miR that is highly or abundantly expressed by a specific state of T cells comprising activated CD3+ T cells, wherein a miR is any one selected from a group consisting of: miR-150-5p, miR-142-3p, miR-29b-3p, miR-342-3p, let-7g-5p, or miR-155-5p. In some embodiments, an mRNA of the disclosure comprising one or more miR binding site(s) to one or more of miR-150-5p, miR-142-3p, miR-29b-3p, miR-342-3p, let-7g-5p, or miR-155-5p has reduced expression in activated CD3+ T cells. In some embodiments, an mRNA of the disclosure comprises one or more miR binding site(s) that binds at least one miR that is highly or abundantly expressed by a specific state of T cells comprising activated CD3+CD4+ T cells, wherein a miR is any one selected from a group consisting of: miR-150-5p, miR-142-3p, miR-29b-3p, miR-342-3p, let-7g-5p, or miR-155-5p. In some embodiments, an mRNA of the disclosure comprising one or more miR binding site(s) to one or more of miR-150-5p, miR-142-3p, miR-29b-3p, miR-342-3p, let-7g-5p, or miR-155-5p has reduced expression in activated CD3+CD4+ T cells. In some embodiments, an mRNA of the disclosure comprises one or more miR binding site(s) that binds at least one miR that is highly or abundantly expressed by a specific state of T cells comprising activated CD3+CD8+ T cells, wherein a miR is any one selected from a group consisting of: miR-150-5p, miR-142-3p, miR-29b-3p, miR-342-3p, let-7g-5p, miR-155-5p, miR-4454, or miR-7975. In some embodiments, an mRNA of the disclosure comprising one or more miR binding site(s) to one or more of miR-150-5p, miR-142-3p, miR-29b-3p, miR-342-3p, let-7g-5p, miR-155-5p, miR-4454, or miR-7975 has reduced expression in activated CD3+CD8+ T cells.

In some embodiments, an mRNA of the disclosure comprises one or more miR binding site(s) that binds to a miR that is differentially expressed in a target cell comprising one cell subset compared to a plurality of non-target cells comprising at least one or more different cell subsets. In some embodiments, a target cell comprises a T cell subset. In some embodiments, a cell subset is an effector T cell. In some embodiments, a cell subset is a naïve T cell. In some embodiments, a cell subset is a regulatory T cell. In some embodiments, an mRNA of the disclosure comprises one or more miR binding site(s) that binds at least one miR that is differentially expressed by a regulatory T cell compared to a naïve T cell, wherein a miR is any one selected from a group consisting of: miR-146a-5p, miR-21-5p, miR-155-5p, miR-15a-5p, let-7i-5p, miR-16-5p, miR-222-3p, miR-15b-5p, miR-24-3p, or miR-4443. In some embodiments, an mRNA of the disclosure comprising one or more miR binding site(s) to one or more of miR-146a-5p, miR-21-5p, miR-155-5p, miR-15a-5p, let-7i-5p, miR-16-5p, miR-222-3p, miR-15b-5p, miR-24-3p, or miR-4443 results in decreased expression of the mRNA in a regulatory T cell compared to a naive T cell. In some embodiments, an mRNA of the disclosure comprising one or more miR binding site(s) that binds to miR-146a-5p results in decreased expression of the mRNA in a regulatory T cell compared to a naïve T cell.

In some embodiments, an mRNA of the disclosure comprises one or more miR binding site(s) that binds at least one miR that is differentially expressed by a regulatory T cell compared to an effector T cell, wherein a miR is any one selected from a group consisting of: miR-146a-5p, miR-181a-5p, miR-223-3p, miR-15a-5p, miR-4286, miR-378g, miR-93-5p, miR-16-5p, miR-25-3p, or miR-15b-5p. In some embodiments, an mRNA of the disclosure comprising one or more miR binding site(s) to one or more of miR-146a-5p, miR-181a-5p, miR-223-3p, miR-15a-5p, miR-4286, miR-378g, miR-93-5p, miR-16-5p, miR-25-3p, or miR-15b-5p results in decreased expression of the mRNA in a regulatory T cell compared to an effector T cell. In some embodiments, an mRNA of the disclosure comprising one or more miR binding site(s) that binds to miR-146a-5p results in decreased expression of the mRNA in a regulatory T cell compared to an effector T cell.

In some embodiments, an mRNA of the disclosure comprises one or more miR binding site(s) that binds to a miR that is differentially expressed in a target cell of a specific cell state compared to a plurality of non-target cells comprising at least one different cell state. In some embodiments, a target cell is a T cell. In some embodiments, a target cell is a resting T cell. In some embodiments, a target cell is an activated T cell. In some embodiments, an mRNA of the disclosure comprises one or more miR binding site(s) that binds at least one miR that is differentially expressed by an activated CD3+ T cell compared to a resting CD3+ T cell, wherein a miR is any one selected from a group consisting of: miR-155-5p, miR-132-3p, miR-106a-5p, miR-17-5p, miR-19b-3p, miR-19a-3p, miR-24-3p, miR-20a-5p, miR-20b-5p, miR-29a-3p, miR-98-5p, or miR-342-5p. In some embodiments, an mRNA of the disclosure comprising one or more miR binding site(s) to one or more of miR-155-5p, miR-132-3p, miR-106a-5p, miR-17-5p, miR-19b-3p, miR-19a-3p, miR-24-3p, miR-20a-5p, miR-20b-5p, miR-29a-3p, miR-98-5p, or miR-342-5p results in decreased expression of the mRNA in an activated CD3+ T cell compared to a resting CD3+ T cell. In some embodiments, an mRNA of the disclosure comprising one or more miR binding site(s) that binds to miR-155-5p results in decreased expression of the mRNA in an activated CD3+ T cell compared to a resting CD3+ T cell. In some embodiments, an mRNA of the disclosure comprising one or more miR binding site(s) that binds to miR-132-3p results in decreased expression of the mRNA in an activated CD3+ T cell compared to a resting CD3+ T cell.

In some embodiments, an mRNA of the disclosure comprises one or more miR binding site(s) that binds at least one miR that is differentially expressed by an activated CD3+CD4+ T cell compared to a resting CD3+CD4+ T cell, wherein a miR is any one selected from a group consisting of: miR-155-5p, miR-132-3p, miR-106a-5p, miR-17-5p, miR-20a-5p, miR-20b-5p, miR-98-5p, miR-19b-3p, miR-19a-3p, miR-4454, miR-7975, miR-92a-3p, or miR-29a-3p. In some embodiments, an mRNA of the disclosure comprising one or more miR binding site(s) to one or more of miR-155-5p, miR-132-3p, miR-106a-5p, miR-17-5p, miR-20a-5p, miR-20b-5p, miR-98-5p, miR-19b-3p, miR-19a-3p, miR-4454, miR-7975, miR-92a-3p, or miR-29a-3p results in decreased expression of the mRNA in an activated CD3+CD4+ T cell compared to a resting CD3+CD4+ T cell. In some embodiments, an mRNA of the disclosure comprising one or more miR binding site(s) that binds to miR-155-5p results in decreased expression of the mRNA in an activated CD3+ T cell compared to a resting CD3+ T cell. In some embodiments, an mRNA of the disclosure comprising one or more miR binding site(s) that binds to miR-132-3p results in decreased expression of the mRNA in an activated CD3+CD4+ T cell compared to a resting CD3+CD4+ T cell.

In some embodiments, an mRNA of the disclosure comprises one or more miR binding site(s) that binds at least one miR that is differentially expressed by an activated CD3+CD8+ T cell compared to a resting CD3+CD8+ T cell, wherein a miR is any one selected from a group consisting of: miR-155-5p, miR-132-3p, miR-106a-5p, miR-17-5p, miR-20a-5p, miR-20b-5p, miR-4454, miR-7975, let-7a-5p, miR-19a-3p, miR-19b-3p, miR-4443, miR-29b-3p. In some embodiments, an mRNA of the disclosure comprising one or more miR binding site(s) to one or more of miR-155-5p, miR-132-3p, miR-106a-5p, miR-17-5p, miR-20a-5p, miR-20b-5p, miR-4454, miR-7975, let-7a-5p, miR-19a-3p, miR-19b-3p, miR-4443, miR-29b-3p results in decreased expression of the mRNA in an activated CD3+CD8+ T cell compared to a resting CD3+CD8+ T cell. In some embodiments, an mRNA of the disclosure comprising one or more miR binding site(s) that binds to miR-155-5p results in decreased expression of the mRNA in an activated CD3+ T cell compared to a resting CD3+ T cell. In some embodiments, an mRNA of the disclosure comprising one or more miR binding site(s) that binds to miR-132-3p results in decreased expression of the mRNA in an activated CD3+CD4+ T cell compared to a resting CD3+CD4+ T cell.

An mRNA of the disclosure can be engineered for more targeted expression in specific tissues, cell types (e.g., regulatory T cells, naïve T cells, or effector T cells), or biological conditions based on the expression patterns of miRs in the different tissues, cell types, or biological conditions. Through introduction of tissue-specific miR binding sites, an mRNA of the disclosure can be designed for optimal protein expression in a tissue or cell, or in the context of a biological condition.

Some microRNAs are abundant in regulatory T cells but are present in lower levels in naïve or effector T cells (e.g., miR-146a-5p). Thus, engineering miR binding site(s) into an mRNA (e.g., in a 3′UTR region or other region) can effectively target the mRNA for degradation or reduce translation in regulatory T while providing high levels of translation in effector or naïve T cells. This provides an approach for differential targeting of effector and naïve T cells relative to regulatory T cells. In some embodiments, at least one miR-binding site for a miR expressed in lower levels in regulatory T cells relative to effector or naïve T cells ells, (e.g., miR-146a-5p), is engineered into an mRNA encoding cellular receptors (e.g., OX40, IL-7) to promote costimulation and/or development of memory T cells; transcription factors to promote TH1 (e.g., T-Bet, Eomes), TH2 (e.g., CAR-T, GATA-3), or TH17 (e.g., RORγt) polarization; transcription factors to promote development of regulatory T cells (e.g., FOXP3); chimeric signaling molecule (e.g., CTLA4-CD28) to prevent T cell exhaustion and promote tumor-killing function; cell trafficking or homing receptors (e.g., CCR-9, CCR-7, CD62L, integrinαβ).

Some microRNAs are abundant in activated T cells (e.g., CD3+, CD3+4+, or CD3+8+ cells activated with PMA/ionophore) but have decreased expression in unstimulated T cells (e.g., CD3+, CD3+4+, or CD3+8+ cells cultured in media) (e.g., miR-155-5p or miR-132-3p). Thus, engineering miR binding site(s) into an mRNA (e.g., in a 3′UTR region or other region) can effectively target the mRNA for degradation or reduce translation in activated T cells while providing high levels of translation in unstimulated T cells. This provides an approach for differential targeting of activated T cells relative to unstimulated T cells. In some embodiments, at least one miR-binding site for a miR expressed in lower levels in unstimulated T cells relative to activated T cells (e.g., miR-155-5p or miR-132-3p), is engineered into a mRNA encoding cytokines or cytokine signaling molecules (IRF-7, NF-κβ, IFN) to promote T cell activation and function.

In some embodiments, an mRNA of the disclosure comprises at least one miR binding site in the 3′UTR in order to selectively degrade mRNA therapeutics in the immune cells to subdue unwanted immunogenic reactions caused by graft vs host disease. As a non-limiting example, the miR binding site can make an mRNA of the disclosure express FOXP3, thus polarizing T cells to a more Treg phenotype. Non-limiting examples of this miR includes miR146a-5p.

Messenger RNA (mRNA)

In some embodiments, the disclosure provides an mRNA for use in the methods described herein. An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An exemplary 5′ UTR for use in the constructs is shown in SEQ ID NO: 73. An exemplary 3′ UTR for use in the constructs is shown in SEQ ID NO: 74. An exemplary 3′ UTR comprising miR-122 and/or miR-142-3p binding sites for use in the constructs is shown in SEQ ID NO: 75. In one embodiment, hepatocyte expression is reduced by including miR122 binding sites. An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,03′GpppG, m27,03′GppppG, m27,02′GppppG, m7Gpppm7G, m73′dGpppG, m27,03′GpppG, m27,03′GppppG, and m27,02′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

In one embodiment, the polynucleotides of the present disclosure may include a sequence encoding a self-cleaving peptide. The self-cleaving peptide may be, but is not limited to, a 2A peptide. A variety of 2A peptides are known and available in the art and may be used, including e.g., the foot and mouth disease virus (FMDV) 2A peptide, the equine rhinitis A virus 2A peptide, the Thosea asigna virus 2A peptide, and the porcine teschovirus-1 2A peptide. 2A peptides are used by several viruses to generate two proteins from one transcript by ribosome-skipping, such that a normal peptide bond is impaired at the 2A peptide sequence, resulting in two discontinuous proteins being produced from one translation event. As a non-limiting example, the 2A peptide may have the protein sequence: GSGATNFSLLKQAGDVEENPGP (SEQ ID NO: 29), fragments or variants thereof. In one embodiment, the 2A peptide cleaves between the last glycine and last proline. As another non-limiting example, the polynucleotides of the present disclosure may include a polynucleotide sequence encoding the 2A peptide having the protein sequence GSGATNFSLLKQAGDVEENPGP (SEQ ID NO: 29) fragments or variants thereof. One example of a polynucleotide sequence encoding the 2A peptide is: GGAAGCGGAGCTACTAACTTCAGCCTGCTGAAGCAGGCTGGAGACGTGGAGGAGAA CCCTGGACCT (SEQ ID NO: 30). In one illustrative embodiment, a 2A peptide is encoded by the following sequence: 5′-TCCGGACTCAGATCCGGGGATCTCAAAATTGTCGCTCCTGTCAAACAAACTCTTAAC TTTGATTTACTCAAACTGGCTGGGGATGTAGAAAGCAATCCAGGTCCACTC-3′(SEQ ID NO: 31). The polynucleotide sequence of the 2A peptide may be modified or codon optimized by the methods described herein and/or are known in the art.

In one embodiment, this sequence may be used to separate the coding regions of two or more polypeptides of interest. As a non-limiting example, the sequence encoding the F2A peptide may be between a first coding region A and a second coding region B (A-F2Apep-B). The presence of the F2A peptide results in the cleavage of the one long protein between the glycine and the proline at the end of the F2A peptide sequence (NPGP is cleaved to result in NPG and P) thus creating separate protein A (with 21 amino acids of the F2A peptide attached, ending with NPG) and separate protein B (with 1 amino acid, P, of the F2A peptide attached). Likewise, for other 2A peptides (P2A, T2A and E2A), the presence of the peptide in a long protein results in cleavage between the glycine and proline at the end of the 2A peptide sequence (NPGP is cleaved to result in NPG and P). Protein A and protein B may be the same or different peptides or polypeptides of interest. In particular embodiments, protein A is a polypeptide that induces immunogenic cell death and protein B is another polypeptide that stimulates an inflammatory and/or immune response and/or regulates immune responsiveness (as described further below).

### Untranslated Regions (UTRs)

Translation of a polynucleotide comprising an open reading frame encoding a polypeptide can be controlled and regulated by a variety of mechanisms that are provided by various cis-acting nucleic acid structures. For example, naturally-occurring, cis-acting RNA elements that form hairpins or other higher-order (e.g., pseudoknot) intramolecular mRNA secondary structures can provide a translational regulatory activity to a polynucleotide, wherein the RNA element influences or modulates the initiation of polynucleotide translation, particularly when the RNA element is positioned in the 5′ UTR close to the 5′-cap structure (Pelletier and Sonenberg (1985) Cell 40(3):515-526; Kozak (1986) Proc Natl Acad Sci 83:2850-2854).

Untranslated regions (UTRs) are nucleic acid sections of a polynucleotide before a start codon (5′ UTR) and after a stop codon (3′ UTR) that are not translated. In some embodiments, a polynucleotide (e.g., a ribonucleic acid (RNA), e.g., a messenger RNA (mRNA)) of the invention comprising an open reading frame (ORF) encoding an ARG1 polypeptide further comprises UTR (e.g., a 5′ UTR or functional fragment thereof, a 3′ UTR or functional fragment thereof, or a combination thereof).

Cis-acting RNA elements can also affect translation elongation, being involved in numerous frameshifting events (Namy et al., (2004) Mol Cell 13(2):157-168). Internal ribosome entry sequences (IRES) represent another type of cis-acting RNA element that are typically located in 5′ UTRs, but have also been reported to be found within the coding region of naturally-occurring mRNAs (Holcik et al. (2000) Trends Genet 16(10):469-473). In cellular mRNAs, IRES often coexist with the 5′-cap structure and provide mRNAs with the functional capacity to be translated under conditions in which cap-dependent translation is compromised (Gebauer et al., (2012) Cold Spring Harb Perspect Biol 4(7):a012245). Another type of naturally-occurring cis-acting RNA element comprises upstream open reading frames (uORFs). Naturally-occurring uORFs occur singularly or multiply within the 5′ UTRs of numerous mRNAs and influence the translation of the downstream major ORF, usually negatively (with the notable exception of GCN4 mRNA in yeast and ATF4 mRNA in mammals, where uORFs serve to promote the translation of the downstream major ORF under conditions of increased eIF2 phosphorylation (Hinnebusch (2005) Annu Rev Microbiol 59:407-450)). Additional exemplary translational regulatory activities provided by components, structures, elements, motifs, and/or specific sequences comprising polynucleotides (e.g., mRNA) include, but are not limited to, mRNA stabilization or destabilization (Baker & Parker (2004) Curr Opin Cell Biol 16(3):293-299), translational activation (Villalba et al., (2011) Curr Opin Genet Dev 21(4):452-457), and translational repression (Blumer et al., (2002) Mech Dev 110(1-2):97-112). Studies have shown that naturally-occurring, cis-acting RNA elements can confer their respective functions when used to modify, by incorporation into, heterologous polynucleotides (Goldberg-Cohen et al., (2002) J Biol Chem 277(16):13635-13640).

Modified mRNAs Comprising Functional RNA Elements

The present disclosure provides synthetic polynucleotides comprising a modification (e.g., an RNA element), wherein the modification provides a desired translational regulatory activity. In some embodiments, the disclosure provides a polynucleotide comprising a 5′ untranslated region (UTR), an initiation codon, a full open reading frame encoding a polypeptide, a 3′ UTR, and at least one modification, wherein the at least one modification provides a desired translational regulatory activity, for example, a modification that promotes and/or enhances the translational fidelity of mRNA translation. In some embodiments, the desired translational regulatory activity is a cis-acting regulatory activity. In some embodiments, the desired translational regulatory activity is an increase in the residence time of the 43S pre-initiation complex (PIC) or ribosome at, or proximal to, the initiation codon. In some embodiments, the desired translational regulatory activity is an increase in the initiation of polypeptide synthesis at or from the initiation codon. In some embodiments, the desired translational regulatory activity is an increase in the amount of polypeptide translated from the full open reading frame. In some embodiments, the desired translational regulatory activity is an increase in the fidelity of initiation codon decoding by the PIC or ribosome. In some embodiments, the desired translational regulatory activity is inhibition or reduction of leaky scanning by the PIC or ribosome. In some embodiments, the desired translational regulatory activity is a decrease in the rate of decoding the initiation codon by the PIC or ribosome. In some embodiments, the desired translational regulatory activity is inhibition or reduction in the initiation of polypeptide synthesis at any codon within the mRNA other than the initiation codon. In some embodiments, the desired translational regulatory activity is inhibition or reduction of the amount of polypeptide translated from any open reading frame within the mRNA other than the full open reading frame. In some embodiments, the desired translational regulatory activity is inhibition or reduction in the production of aberrant translation products. In some embodiments, the desired translational regulatory activity is a combination of one or more of the foregoing translational regulatory activities.

Accordingly, the present disclosure provides a polynucleotide, e.g., an mRNA, comprising an RNA element that comprises a sequence and/or an RNA secondary structure(s) that provides a desired translational regulatory activity as described herein. In some aspects, the mRNA comprises an RNA element that comprises a sequence and/or an RNA secondary structure(s) that promotes and/or enhances the translational fidelity of mRNA translation. In some aspects, the mRNA comprises an RNA element that comprises a sequence and/or an RNA secondary structure(s) that provides a desired translational regulatory activity, such as inhibiting and/or reducing leaky scanning. In some aspects, the disclosure provides an mRNA that comprises an RNA element that comprises a sequence and/or an RNA secondary structure(s) that inhibits and/or reduces leaky scanning thereby promoting the translational fidelity of the mRNA.

In some embodiments, the RNA element comprises natural and/or modified nucleotides. In some embodiments, the RNA element comprises of a sequence of linked nucleotides, or derivatives or analogs thereof, that provides a desired translational regulatory activity as described herein. In some embodiments, the RNA element comprises a sequence of linked nucleotides, or derivatives or analogs thereof, that forms or folds into a stable RNA secondary structure, wherein the RNA secondary structure provides a desired translational regulatory activity as described herein. RNA elements can be identified and/or characterized based on the primary sequence of the element (e.g., GC-rich element), by RNA secondary structure formed by the element (e.g. stem-loop), by the location of the element within the RNA molecule (e.g., located within the 5′ UTR of an mRNA), by the biological function and/or activity of the element (e.g., “translational enhancer element”), and any combination thereof.

In some aspects, the disclosure provides an mRNA having one or more structural modifications that inhibits leaky scanning and/or promotes the translational fidelity of mRNA translation, wherein at least one of the structural modifications is a GC-rich RNA element. In some aspects, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In any of the foregoing or related aspects, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is 70-80% cytosine, 60-70% cytosine, 50%-60% cytosine, 40-50% cytosine, 30-40% cytosine bases. In any of the foregoing or related aspects, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is about 80% cytosine, about 70% cytosine, about 60% cytosine, about 50% cytosine, about 40% cytosine, or about 30% cytosine.

In any of the foregoing or related aspects, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is 70-80% cytosine, 60-70% cytosine, 50%-60% cytosine, 40-50% cytosine, or 30-40% cytosine. In any of the foregoing or related aspects, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of 3, 4, 5, 6, 7, 8, 9, 10, 11, 12, 13, 14, 15, 16, 17, 18, 19, or 20 nucleotides, or derivatives or analogs thereof, linked in any order, wherein the sequence composition is >50% cytosine. In some embodiments, the sequence composition is >55% cytosine, >60% cytosine, >65% cytosine, >70% cytosine, >75% cytosine, >80% cytosine, >85% cytosine, or >90% cytosine.

In other aspects, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of about 3-30, 5-25, 10-20, 15-20 or about 20, about 15, about 12, about 10, about 6 or about 3 nucleotides, or derivatives or analogues thereof, wherein the sequence comprises a repeating GC-motif, wherein the repeating GC-motif is [CCG]n, wherein n=1 to 10, n=2 to 8, n=3 to 6, or n=4 to 5. In some embodiments, the sequence comprises a repeating GC-motif [CCG]n, wherein n=1, 2, 3, 4 or 5. In some embodiments, the sequence comprises a repeating GC-motif [CCG]n, wherein n=1, 2, or 3. In some embodiments, the sequence comprises a repeating GC-motif [CCG]n, wherein n=1. In some embodiments, the sequence comprises a repeating GC-motif [CCG]n, wherein n=2. In some embodiments, the sequence comprises a repeating GC-motif [CCG]n, wherein n=3. In some embodiments, the sequence comprises a repeating GC-motif [CCG]n, wherein n=4. In some embodiments, the sequence comprises a repeating GC-motif [CCG]n, wherein n=5.

In another aspect, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element comprises any one of the sequences set forth in Table 9. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located about 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In other aspects, the disclosure provides a modified mRNA comprising at least one modification, wherein at least one modification is a GC-rich RNA element comprising the sequence V1 [CCCCGGCGCC (SEQ ID NO: 33)] as set forth in Table 9, or derivatives or analogs thereof, preceding a Kozak consensus sequence in the 5′ UTR of the mRNA. In some embodiments, the GC-rich element comprises the sequence V1 as set forth in Table 9 located immediately adjacent to and upstream of the Kozak consensus sequence in the 5′ UTR of the mRNA. In some embodiments, the GC-rich element comprises the sequence V1 as set forth in Table 9 located 1, 2, 3, 4, 5, 6, 7, 8, 9 or 10 bases upstream of the Kozak consensus sequence in the 5′ UTR of the mRNA. In other embodiments, the GC-rich element comprises the sequence V1 as set forth in Table 9 located 1-3, 3-5, 5-7, 7-9, 9-12, or 12-15 bases upstream of the Kozak consensus sequence in the 5′ UTR of the mRNA.

In other aspects, the disclosure provides a modified mRNA comprising at least one modification, wherein at least one modification is a GC-rich RNA element comprising the sequence V2 [CCCCGGC (SEQ ID NO: 34)] as set forth in Table 9, or derivatives or analogs thereof, preceding a Kozak consensus sequence in the 5′ UTR of the mRNA. In some embodiments, the GC-rich element comprises the sequence V2 as set forth in Table 9 located immediately adjacent to and upstream of the Kozak consensus sequence in the 5′ UTR of the mRNA. In some embodiments, the GC-rich element comprises the sequence V2 as set forth in Table 9 located 1, 2, 3, 4, 5, 6, 7, 8, 9 or 10 bases upstream of the Kozak consensus sequence in the 5′ UTR of the mRNA. In other embodiments, the GC-rich element comprises the sequence V2 as set forth in Table 9 located 1-3, 3-5, 5-7, 7-9, 9-12, or 12-15 bases upstream of the Kozak consensus sequence in the 5′ UTR of the mRNA.

In other aspects, the disclosure provides a modified mRNA comprising at least one modification, wherein at least one modification is a GC-rich RNA element comprising the sequence EK [GCCGCC (SEQ ID NO: 35)] as set forth in Table 9, or derivatives or analogs thereof, preceding a Kozak consensus sequence in the 5′ UTR of the mRNA. In some embodiments, the GC-rich element comprises the sequence EK as set forth in Table 9 located immediately adjacent to and upstream of the Kozak consensus sequence in the 5′ UTR of the mRNA. In some embodiments, the GC-rich element comprises the sequence EK as set forth in Table 9 located 1, 2, 3, 4, 5, 6, 7, 8, 9 or 10 bases upstream of the Kozak consensus sequence in the 5′ UTR of the mRNA. In other embodiments, the GC-rich element comprises the sequence EK as set forth in Table 9 located 1-3, 3-5, 5-7, 7-9, 9-12, or 12-15 bases upstream of the Kozak consensus sequence in the 5′ UTR of the mRNA.

In yet other aspects, the disclosure provides a modified mRNA comprising at least one modification, wherein at least one modification is a GC-rich RNA element comprising the sequence V1 [CCCCGGCGCC (SEQ ID NO: 33)] as set forth in Table 9, or derivatives or analogs thereof, preceding a Kozak consensus sequence in the 5′ UTR of the mRNA, wherein the 5′ UTR comprises the following sequence shown in Table 9:

GGGAAAUAAGAGAGAAAAGAAGAGUAAGAAGAAAUAUAAGA (SEQ ID NO: 36). The skilled artisan will of course recognize that all Us in the RNA sequences described herein will be Ts in a corresponding template DNA sequence, for example, in DNA templates or constructs from which mRNAs of the disclosure are transcribed, e.g., via IVT.

In some embodiments, the GC-rich element comprises the sequence V1 as set forth in Table 9 located immediately adjacent to and upstream of the Kozak consensus sequence in the 5′ UTR sequence shown in Table 9. In some embodiments, the GC-rich element comprises the sequence V1 as set forth in Table 2 located 1, 2, 3, 4, 5, 6, 7, 8, 9 or 10 bases upstream of the Kozak consensus sequence in the 5′ UTR of the mRNA, wherein the 5′ UTR comprises the following sequence shown in Table 9: GGGAAAUAAGAGAGAAAAGAAGAGUAAGAAGAAAUAUAAGA (SEQ ID NO: 36).

In other embodiments, the GC-rich element comprises the sequence V1 as set forth in Table 9 located 1-3, 3-5, 5-7, 7-9, 9-12, or 12-15 bases upstream of the Kozak consensus sequence in the 5′ UTR of the mRNA, wherein the 5′ UTR comprises the following sequence shown in Table 9: GGGAAAUAAGAGAGAAAAGAAGAGUAAGAAGAAAUAUAAGA (SEQ ID NO: 36).

In some embodiments, the 5′ UTR comprises the following sequence set forth in Table 9: GGGAAAUAAGAGAGAAAAGAAGAGUAAGAAGAAAUAUAAGACCCCGGCGCCGCC ACC (SEQ ID NO: 37)

TABLE 9

| 5′ UTRs | 5′ UTR Sequence |
|---|---|
| 001-UTR | GGGAAAUAAGAGAGAAAAGAAGAGUAAGAAGAA |
| | AUAUAAGA (SEQ ID NO: 36) |
| Standard | GGGAAAUAAGAGAGAAAAGAAGAGUAAGAAGAA |
| | AUAUAAGAGCCACC (SEQ ID NO: 38) |
| V1-UTR | GGGAAAUAAGAGAGAAAAGAAGAGUAAGAAGAA |
| | AUAUAAGACCCCGGCGCCGCCACC (SEQ ID |
| | NO: 39) |
| V2-UTR | GGGAAAUAAGAGAGAAAAGAAGAGUAAGAAGAA |
| | AUAUAAGACCCCGGCGCCACC (SEQ ID NO: |
| | 40) |
| GC-Rich | |
| RNA Elements | Sequence |
| K0 | [GCCA/GCC] (SEQ ID NO: 32) |
| (Traditional | |
| Kozak | |
| consensus) | |
| EK | [GCCGCC] (SEQ ID NO: 35) |
| V1 | [CCCCGGCGCC] (SEQ ID NO: 33) |
| V2 | [CCCCGGC] (SEQ ID NO: 34) |
| (CCG)ₙ, where | [CCG]ₙ |
| n = 1-10 | |
| (GCC)ₙ, where | [GCC]ₙ |
| n = 1-10 | |

In another aspect, the disclosure provides a modified mRNA comprising at least one modification, wherein at least one modification is a GC-rich RNA element comprising a stable RNA secondary structure comprising a sequence of nucleotides, or derivatives or analogs thereof, linked in an order which forms a hairpin or a stem-loop. In one embodiment, the stable RNA secondary structure is upstream of the Kozak consensus sequence. In another embodiment, the stable RNA secondary structure is located about 30, about 25, about 20, about 15, about 10, or about 5 nucleotides upstream of the Kozak consensus sequence. In another embodiment, the stable RNA secondary structure is located about 20, about 15, about 10 or about 5 nucleotides upstream of the Kozak consensus sequence. In another embodiment, the stable RNA secondary structure is located about 5, about 4, about 3, about 2, about 1 nucleotides upstream of the Kozak consensus sequence. In another embodiment, the stable RNA secondary structure is located about 15-30, about 15-20, about 15-25, about 10-15, or about 5-10 nucleotides upstream of the Kozak consensus sequence. In another embodiment, the stable RNA secondary structure is located 12-15 nucleotides upstream of the Kozak consensus sequence. In another embodiment, the stable RNA secondary structure has a deltaG of about −30 kcal/mol, about −20 to −30 kcal/mol, about −20 kcal/mol, about −10 to −20 kcal/mol, about −10 kcal/mol, about −5 to ˜10 kcal/mol.

In another embodiment, the modification is operably linked to an open reading frame encoding a polypeptide and wherein the modification and the open reading frame are heterologous.

In another embodiment, the sequence of the GC-rich RNA element is comprised exclusively of guanine (G) and cytosine (C) nucleobases.

RNA elements that provide a desired translational regulatory activity as described herein can be identified and characterized using known techniques, such as ribosome profiling. Ribosome profiling is a technique that allows the determination of the positions of PICs and/or ribosomes bound to mRNAs (see e.g., Ingolia et al., (2009) Science 324(5924):218-23, incorporated herein by reference). The technique is based on protecting a region or segment of mRNA, by the PIC and/or ribosome, from nuclease digestion. Protection results in the generation of a 30-bp fragment of RNA termed a ‘footprint’. The sequence and frequency of RNA footprints can be analyzed by methods known in the art (e.g., RNA-seq). The footprint is roughly centered on the A-site of the ribosome. If the PIC or ribosome dwells at a particular position or location along an mRNA, footprints generated at these position would be relatively common. Studies have shown that more footprints are generated at positions where the PIC and/or ribosome exhibits decreased processivity and fewer footprints where the PIC and/or ribosome exhibits increased processivity (Gardin et al., (2014) eLife 3:e03735). In some embodiments, residence time or the time of occupancy of the PIC or ribosome at a discrete position or location along a polynucleotide comprising any one or more of the RNA elements described herein is determined by ribosome profiling.

A UTR can be homologous or heterologous to the coding region in a polynucleotide. In some embodiments, the UTR is homologous to the ORF encoding the ARG1 polypeptide. In some embodiments, the UTR is heterologous to the ORF encoding the ARG1 polypeptide. In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG (SEQ ID NO: 41), where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Exemplary UTRs of the application include, but are not limited to, one or more 5′UTR and/or 3′UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245α polypeptide); an albumin (e.g., human albumin7); a HSD17B4 (hydroxysteroid (17-13) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′UTR containing a strong Kozak translational initiation signal; a cytochrome b-245α polypeptide (CYBA) 5′ UTR; a hydroxysteroid (17-β) dehydrogenase (HSD17B4) 5′ UTR; a Tobacco etch virus (TEV) 5′ UTR; a Venezuelan equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; a-globin 3′UTR; a DEN 3′ UTR; a PAV barley yellow dwarf virus (BYDV-PAV) 3′ UTR; an elongation factor 1α1 (EEF1A1) 3′ UTR; a manganese superoxide dismutase (MnSOD) 3′ UTR; a β subunit of mitochondrial H(+)-ATP synthase (0-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the polynucleotides of the invention comprise a 5′ UTR and/or a 3′ UTR selected from any of the UTRs disclosed herein. In some embodiments, the 5′ UTR comprises:

5′ UTR-001 (Upstream UTR)
(SEQ ID NO: 42)
(GGGAAAUAAGAGAGAAAAGAAGAGUAAGAAGAAAUAUAAGAGCCACC);
5′ UTR-002 (Upstream UTR)
(SEQ ID NO: 43)
(GGGAGAUCAGAGAGAAAAGAAGAGUAAGAAGAAAUAUAAGAGCCACC);
5′ UTR-003 (Upstream UTR)
(See W02016/100812);
5′ UTR-004 (Upstream UTR)
(SEQ ID NO: 44)
(GGGAGACAAGCUUGGCAUUCCGGUACUGUUGGUAAAGCCACC);
5′ UTR-005 (Upstream UTR)
(SEQ ID NO: 45)
(GGGAGAUCAGAGAGAAAAGAAGAGUAAGAAGAAAUAUAAGAGCCACC);
5′ UTR-006 (Upstream UTR)
(See WO2016/100812);
5′ UTR-007 (Upstream UTR)
(SEQ ID NO: 46)
(GGGAGACAAGCUUGGCAUUCCGGUACUGUUGGUAAAGCCACC);
5′ UTR-008 (Upstream UTR)
(SEQ ID NO: 47)
(GGGAAUUAACAGAGAAAAGAAGAGUAAGAAGAAAUAUAAGAGCCACC);
5′ UTR-009 (Upstream UTR)
(SEQ ID NO: 48)
(GGGAAAUUAGACAGAAAAGAAGAGUAAGAAGAAAUAUAAGAGCCACC);
5′ UTR-010, Upstream
(SEQ ID NO: 49)
(GGGAAAUAAGAGAGUAAAGAACAGUAAGAAGAAAUAUAAGAGCCACC);

In some embodiments, the 3′ UTR comprises:

142-3p 3′ UTR (UTR including miR142-3p
binding site)
(SEQ ID NO: 58)
(UGAUAAUAGUCCAUAAAGUAGGAAACACUACAGCUGGAGCCUCGGUGGC
CAUGCUUCUUGCCCCUUGGGCCUCCCCCCAGCCCCUCCUCCCCUUCCUGC
ACCCGUACCCCCGUGGUCUUUGAAUAAAGUCUGAGUGGGCGGC);
142-3p 3′ UTR (UTR including miR142-3p
binding site)
(SEQ ID NO: 59)
(UGAUAAUAGGCUGGAGCCUCGGUGGCUCCAUAAAGUAGGAAACACUACA
CAUGCUUCUUGCCCCUUGGGCCUCCCCCCAGCCCCUCCUCCCCUUCCUGC
ACCCGUACCCCCGUGGUCUUUGAAUAAAGUCUGAGUGGGCGGC);
or
142-3p 3′ UTR (UTR including miR142-3p
binding site)
(SEQ ID NO: 60)
(UGAUAAUAGGCUGGAGCCUCGGUGGCCAUGCUUCUUGCCCCUUCCAUAA
AGUAGGAAACACUACAUGGGCCUCCCCCCAGCCCCUCCUCCCCUUCCUGC
ACCCGUACCCCCGUGGUCUUUGAAUAAAGUCUGAGUGGGCGGC);
142-3p 3′ UTR (UTR including miR142-3p
binding site)
(SEQ ID NO: 61)
(UGAUAAUAGGCUGGAGCCUCGGUGGCCAUGCUUCUUGCCCCUUGGGCCU
CCCCCCAGUCCAUAAAGUAGGAAACACUACACCCCUCCUCCCCUUCCUGC
ACCCGUACCCCCGUGGUCUUUGAAUAAAGUCUGAGUGGGCGGC);
142-3p 3′ UTR (UTR including miR142-3p
binding site)
(SEQ ID NO: 62)
(UGAUAAUAGGCUGGAGCCUCGGUGGCCAUGCUUCUUGCCCCUUGGGCCU
CCCCCCAGCCCCUCCUCCCCUUCUCCAUAAAGUAGGAAACACUACACUGC
ACCCGUACCCCCGUGGUCUUUGAAUAAAGUCUGAGUGGGCGGC);
142-3p 3′ UTR (UTR including miR142-3p
binding site)
(SEQ ID NO: 63)
(UGAUAAUAGGCUGGAGCCUCGGUGGCCAUGCUUCUUGCCCCUUGGGCCU
CCCCCCAGCCCCUCCUCCCCUUCCUGCACCCGUACCCCCUCCAUAAAGUA
GGAAACACUACAGUGGUCUUUGAAUAAAGUCUGAGUGGGCGGC).
142-3p 3′ UTR (UTR including miR142-3p
binding site)
(SEQ ID NO: 64)
(UGAUAAUAGGCUGGAGCCUCGGUGGCCAUGCUUCUUGCCCCUUGGGCCU
CCCCCCAGCCCCUCCUCCCCUUCCUGCACCCGUACCCCCGUGGUCUUUGA
AUAAAGUUCCAUAAAGUAGGAAACACUACACUGAGUGGGCGGC);
3′ UTR-018 (See SEQ ID NO: 65);
3′ UTR (miR142 and miR126 binding
sites variant 1)
(SEQ ID NO: 66)
(UGAUAAUAGUCCAUAAAGUAGGAAACACUACAGCUGGAGCCUCGGUGGC
CAUGCUUCUUGCCCCUUGGGCCUCCCCCCAGCCCCUCCUCCCCUUCCUGC
ACCCGUACCCCCCGCAUUAUUACUCACGGUACGAGUGGUCUUUGAAUAAA
GUCUGAGUGGGCGGC)
3′ UTR (miR142 and miR126 binding sites
variant 2)
(SEQ ID NO: 67)
(UGAUAAUAGUCCAUAAAGUAGGAAACACUACAGCUGGAGCCUCGGUGGC
CUAGCUUCUUGCCCCUUGGGCCUCCCCCCAGCCCCUCCUCCCCUUCCUGC
ACCCGUACCCCCCGCAUUAUUACUCACGGUACGAGUGGUCUUUGAAUAAA
GUCUGAGUGGGCGGC);
or
3′ UTR (miR142-3p binding site variant 3)
(SEQ ID NO: 68)
UGAUAAUAGGCUGGAGCCUCGGUGGCCUAGCUUCUUGCCCCUUGGGCCUC
CCCCCAGCCCCUCCUCCCCUUCCUGCACCCGUACCCCCUCCAUAAAGUAG
GAAACACUACAGUGGUCUUUGAAUAAAGUCUGAGUGGGCGGC.

In certain embodiments, the 5′ UTR and/or 3′ UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′ UTR sequences comprising any of SEQ ID NOs: 36-40 or, 42-57 and/or 3′ UTR sequences comprises any of SEQ ID NOs: 58-68 or 73-75, and any combination thereof.

In certain embodiments, the 5′ UTR and/or 3′ UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′ UTR sequences comprising any of SEQ ID NO: 36, SEQ ID NO: 37, SEQ ID NO: 38, or SEQ ID NO: 39 and/or 3′ UTR sequences comprises any of SEQ ID NO: 58, SEQ ID NO: 59, SEQ ID NO: 60, SEQ ID NO: 61, SEQ ID NO: 62, SEQ ID NO: 63, SEQ ID NO: 64, or SEQ ID NO: 65, and any combination thereof.

In some embodiments, the 5′ UTR comprises a sequence set forth SEQ ID NO: 36, SEQ ID NO: 37, SEQ ID NO: 38, or SEQ ID NO: 39. In some embodiments, the 3′ UTR comprises a sequence set forth in SEQ ID NO: 58, SEQ ID NO: 59, SEQ ID NO: 60, SEQ ID NO: 61, SEQ ID NO: 62, SEQ ID NO: 63, SEQ ID NO: 64, or SEQ ID NO: 65. In some embodiments, the 5′ UTR comprises a sequence set forth SEQ ID NO: 36, SEQ ID NO: 37, SEQ ID NO: 38, or SEQ ID NO: 39 and the 3′ UTR comprises a sequence set forth in SEQ ID NO: 58, SEQ ID NO: 59, SEQ ID NO: 60, SEQ ID NO: 61, SEQ ID NO: 62, SEQ ID NO: 63, SEQ ID NO: 64, or SEQ ID NO: 65.

The polynucleotides of the invention can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE.

In one aspect, a TEE is a conserved element in a UTR that can promote translational activity of a nucleic acid such as, but not limited to, cap-dependent or cap-independent translation.

### 5′ Capping

It is desirable to manufacture therapeutic RNAs enzymatically using in vitro transcription (IVT). In general, a DNA-dependent RNA polymerase transcribes a DNA template containing an appropriate promoter into an RNA transcript. The poly(A) tail can be generated co-transcriptionally by incorporating a poly(T) tract in the template DNA or separately by using a poly(A) polymerase. Eukaryotic mRNAs start with a 5′ cap (e.g., a 5′ m7GpppX cap). Typically, the 5′ cap begins with an inverted G with N⁷Me (required for eIF4E binding). A preferred cap, Cap1 contains 2′OMe at the +1 position) followed by any nucleoside at +2 position. This cap can be installed post-transcriptionally, e.g., enzymatically (after transcription) or co-transcriptionally (during transcription).

Post-transcriptional capping can be carried out using the vaccinia capping enzyme and allows for complete capping of the RNA, generating a cap 0 structure on RNA carrying a 5′ terminal triphosphate or diphosphate group, the cap 0 structure being required for efficient translation of the mRNA in vivo. The cap 0 structure can then be further modified into cap 1 using a cap-specific 2′O methyltransferase. Vaccinia capping enzyme and 2′O methyltransferase have been used to generate cap 0 and cap 1 structures on in vitro transcripts, for example, for use in transfecting eukaryotic cells or in mRNA therapeutic applications to drive protein synthesis. While post-transcriptional capping by vaccinia capping enzymes can yield either Cap 0 or Cap 1 structures, it is an expensive process when utilized for large-scale mRNA production, for example, vaccinia is costly and in limited supply and there can be difficulties in purifying an IVT mRNA (e.g., removing S-adenosylmethionine (SAM) and 20-methyltransferase). Moreover, capping can be incomplete due to inaccessibility of structured 5′ ends.

Co-transcriptional capping using a cap analog has certain advantages over vaccinia capping, for example, the process requires a simpler workflow (e.g., no need for a purification step between transcription and capping). Traditional co-transcriptional capping methods utilize the dinucleotide ARCA (anti-reverse cap analog) and yield Cap 0 structures. ARCA capping has drawbacks, however, for example, the resulting Cap 0 structures can be immunogenic and the process often results in low yields and/or poorly capped material. Another potential drawback of this approach is a theoretical capping efficiency of <100%, due to competition from the GTP for the starting nucleotide. For example, co-transcriptional capping using ARCA typically requires a 10:1 ratio of ARCA:GTP to achieve >90% capping (needed to outcompete GTP for initiation).

In some embodiments, mRNAs of the disclosure are comprised of trinucleotide mRNA cap analogs, prepared using co-transcriptional capping methods (e.g., featuring T7 RNA polymerase) for the in vitro synthesis of mRNA. Use of a trinucleotide cap analog may provide a solution to several of the above-described problems associated with vaccinia or ARCA capping. In addition, the methods of co-transcriptional capping described provide flexibility in modifying the penultimate nucleobase which may alter binding behavior, or affect the affinity of these caps towards decapping enzymes, or both, thus potentially improving stability of the respective mRNA. An exemplary trinucleotide for use in the herein-described co-transcriptional capping methods is the m7GpppAG (GAG) trinucleotide. Use of this trinucleotide results in the nucleotide at the +1 position being A instead of G. Both +1G and +1A are caps that can be found in naturally-occurring mRNAs.

T7 RNA polymerase prefers to initiate with 5′ GTP. Accordingly, Most conventional mRNA transcripts start with 5′- [symbol] (based on transcription from a T7 promoter sequence such as 5′TAATACGACTCACTATA [symbol] NNNNNNNNN . . . 3′ (SEQ ID NO: 69) (TATA being referred to as the “TATA box”). T7 RNA polymerase typically transcribes DNA downstream of a T7 promoter (5′ TAATACGACTCACTATAG 3′, (SEQ ID NO: 70) referencing the coding strand). T7 polymerase starts transcription at the underlined G in the promoter sequence. The polymerase then transcribes using the opposite strand as a template from 5′->3′. The first base in the transcript will be a G.

The herein-described processes capitalize on the fact that the T7 enzyme has limited initiation activity with the single nucleotide ATP, driving T7 to initiate with the trinucleotide rather than ATP. The process thus generates an mRNA product with >90% functional cap post-transcription. The process is an efficient “one-pot” mRNA production method that includes, for example, the GAG trinucleotide (GpppAG; ^{m}GpppAₘG) in equimolar concentration with the NTPs, GTP, ATP, CTP and UTP. The process features an “A-start” DNA template that initiates transcription with 5′ adenosine (A). As defined herein, “A-start” and “G-start” DNA templates are double-stranded DNA having requisite nucleosides in the template strand, such that the coding strand (and corresponding mRNA) begin with A or G, respectively. For example, a G-start DNA template features a template strand having the nucleobases CC complementary to GG immediately downstream of the TATA box in the T7 promoter (referencing the coding strand), and an A-start DNA template features a template strand having the nucleobases TC complementary to the AG immediately downstream of the TATA box in the T7 promoter (referencing the coding strand).

An exemplary T7 promoter sequence featured in an A-start DNA template of the present disclosure is depicted here:

5′ TAATACGACTCACTATA [symbol] NNNNNNNNNN... 3′
3′ ATTATGCTGAGTGATAT [symbol] NNNNNNNNNN... 3′

The trinucleotide-based capping methods described herein provide flexibility in dictating the penultimate nucleobase. The trinucleotide capping methods of the present disclosure provide efficient production of capped mRNA, for example, 95-98% capped mRNA with a natural cap 1 structure.

Poly-A tails

In some embodiments, a polynucleotide comprising an mRNA encoding a polypeptide of the present disclosure further comprises a poly A tail. In further embodiments, terminal groups on the poly-A tail can be incorporated for stabilization. In other embodiments, a poly-A tail comprises des-3′ hydroxyl tails. The useful poly-A tails can also include structural moieties or 2′-Omethyl modifications as taught by Li et al. (2005) Current Biology 15:1501-1507.

In one embodiment, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1.100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3 ‘-end using modified nucleotides at the 3’-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present disclosure are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone.

### Start Codon Region

In some embodiments, an mRNA of the present disclosure further comprises regions that are analogous to or function like a start codon region.

In some embodiments, the translation of a polynucleotide initiates on a codon which is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG. See Touriol et al. (2003) Biology of the Cell 95:169-178 and Matsuda and Mauro (2010) PLoS ONE 5:11. As a non-limiting example, the translation of a polynucleotide begins on the alternative start codon ACG. As another non-limiting example, polynucleotide translation begins on the alternative start codon CUG. As yet another non-limiting example, the translation of a polynucleotide begins on the alternative start codon GUG.

Nucleotides flanking a codon that initiates translation such as, but not limited to, a start codon or an alternative start codon, are known to affect the translation efficiency, the length and/or the structure of the polynucleotide. See, e.g., Matsuda and Mauro (2010) PLoS ONE 5:11. Masking any of the nucleotides flanking a codon that initiates translation can be used to alter the position of translation initiation, translation efficiency, length and/or structure of a polynucleotide.

In some embodiments, a masking agent is used near the start codon or alternative start codon in order to mask or hide the codon to reduce the probability of translation initiation at the masked start codon or alternative start codon. Non-limiting examples of masking agents include antisense locked nucleic acids (LNA) polynucleotides and exon-junction complexes (EJCs). See, e.g., Matsuda and Mauro (2010) PLoS ONE 5:11, describing masking agents LNA polynucleotides and EJCs.

In another embodiment, a masking agent is used to mask a start codon of a polynucleotide in order to increase the likelihood that translation will initiate on an alternative start codon. In some embodiments, a masking agent is used to mask a first start codon or alternative start codon in order to increase the chance that translation will initiate on a start codon or alternative start codon downstream to the masked start codon or alternative start codon.

In some embodiments, a start codon or alternative start codon is located within a perfect complement for a miR binding site. The perfect complement of a miR binding site can help control the translation, length and/or structure of the polynucleotide similar to a masking agent. As a non-limiting example, the start codon or alternative start codon is located in the middle of a perfect complement for a miR-122 binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide is removed from the polynucleotide sequence in order to have the translation of the polynucleotide begin on a codon which is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence in order to have translation initiate on a downstream start codon or alternative start codon. The polynucleotide sequence where the start codon was removed can further comprise at least one masking agent for the downstream start codon and/or alternative start codons in order to control or attempt to control the initiation of translation, the length of the polynucleotide and/or the structure of the polynucleotide.

### Stop Codon Region

In some embodiments, mRNA of the present disclosure can further comprise at least one stop codon or at least two stop codons before the 3′ untranslated region (UTR). The stop codon can be selected from UGA, UAA, and UAG. In some embodiments, the polynucleotides of the present disclosure include the stop codon UGA and one additional stop codon. In a further embodiment the addition stop codon can be UAA. In another embodiment, the polynucleotides of the present disclosure include three stop codons, four stop codons, or more.

Adjusted Uracil Content

In some embodiments of the disclosure, an mRNA may have adjusted uracil content. In some embodiments, the uracil content of the open reading frame (ORF) of the polynucleotide encoding a therapeutic polypeptide relative to the theoretical minimum uracil content of a nucleotide sequence encoding the therapeutic polypeptide (% U_{TM}), is between about 100% and about 150. In some embodiments, the uracil content of the ORF is between about 105% and about 145%, about 105% and about 140%, about 110% and about 140%, about 110% and about 145%, about 115% and about 135%, about 105% and about 135%, about 110% and about 135%, about 115% and about 145%, or about 115% and about 140% of the theoretical minimum uracil content in the corresponding wild-type ORF (% U_{TM}). In other embodiments, the uracil content of the ORF is between about 117% and about 134% or between 118% and 132% of the % U_{TM}. In some embodiments, the uracil content of the ORF encoding a polypeptide is about 115%, about 120%, about 125%, about 130%, about 135%, about 140%, about 145%, or about 150% of the % U_{TM}. In this context, the term “uracil” can refer to an alternative uracil and/or naturally occurring uracil.

In some embodiments, the uracil content of the ORF of the polynucleotide relative to the uracil content of the corresponding wild-type ORF (% U_{WT}) is less than 100%. In some embodiments, the % U_{WT} of the polynucleotide is less than about 95%, less than about 90%, less than about 85%, less than 80%, less than 79%, less than 78%, less than 77%, less than 76%, less than 75%, less than 74%, or less than 73%. In some embodiments, the % U_{WT} of the polynucleotide is between 65% and 73%.

In some embodiments, the uracil content in the ORF of the mRNA encoding a is less than about 50%, about 40%, about 30%, or about 20% of the total nucleobase content in the ORF. In some embodiments, the uracil content in the ORF is between about 15% and about 25% of the total nucleobase content in the ORF. In other embodiments, the uracil content in the ORF is between about 20% and about 30% of the total nucleobase content in the ORF. In one embodiment, the uracil content in the ORF of the mRNA encoding a polypeptide is less than about 20% of the total nucleobase content in the open reading frame. In this context, the term “uracil” can refer to an alternative uracil and/or naturally occurring uracil.

In further embodiments, the ORF of the mRNA encoding a polypeptide having adjusted uracil content has increased cytosine (C), guanine (G), or guanine/cytosine (G/C) content (absolute or relative). In some embodiments, the overall increase in C, G, or G/C content (absolute or relative) of the ORF is at least about 2%, at least about 3%, at least about 4%, at least about 5%, at least about 6%, at least about 7%, at least about 10%, at least about 15%, at least about 20%, at least about 40%, at least about 50%, at least about 60%, at least about 70%, at least about 80%, at least about 90%, at least about 95%, or at least about 100% relative to the G/C content (absolute or relative) of the wild-type ORF. In some embodiments, the G, the C, or the G/C content in the ORF is less than about 100%, less than about 90%, less than about 85%, or less than about 80% of the theoretical maximum G, C, or G/C content of the nucleotide sequence encoding the PBDG polypeptide (% G_{TMX}; % C_{TMX}, or % G/C_{TMX}). In other embodiments, the G, the C, or the G/C content in the ORF is between about 70% and about 80%, between about 71% and about 79%, between about 71% and about 78%, or between about 71% and about 77% of the % G_{TMX}, % C_{TMX}, or % G/C_{TMX}. In some embodiments, the guanine content of the ORF of the polynucleotide with respect to the theoretical maximum guanine content of a nucleotide sequence encoding the polypeptide (% G_{TMX}) is at least 69%, at least 70%, at least 75%, at least about 80%, at least about 85%, at least about 90%, at least about 95%, or about 100%. In some embodiments, the % G_{TMX} of the polynucleotide is between about 70% and about 80%, between about 71% and about 79%, between about 71% and about 78%, or between about 71% and about 77%. In some embodiments, the cytosine content of the ORF of the polynucleotide relative to the theoretical maximum cytosine content of a nucleotide sequence encoding the polypeptide (% C_{TMX}) is at least 59%, at least 60%, at least about 65%, at least about 70%, at least about 75%, at least about 80%, at least about 85%, at least about 90%, at least about 95%, or about 100%. In some embodiments, the % C_{TMX} of the ORF of the polynucleotide is between about 60% and about 80%, between about 62% and about 80%, between about 63% and about 79%, or between about 68% and about 76%. In some embodiments, the guanine and cytosine content (G/C) of the ORF of the polynucleotide relative to the theoretical maximum G/C content in a nucleotide sequence encoding the polypeptide (% G/C_{TMX}) is at least about 81%, at least about 85%, at least about 90%, at least about 95%, or about 100%. In some embodiments, the % G/C_{TMX} in the ORF of the polynucleotide is between about 80% and about 100%, between about 85% and about 99%, between about 90% and about 97%, or between about 91% and about 96%. In some embodiments, the G/C content in the ORF of the polynucleotide relative to the G/C content in the corresponding wild-type ORF (% G/C_{WT}) is at least 102%, at least 103%, at least 104%, at least 105%, at least 106%, at least 107%, at least 110%, at least 115%, or at least 120%. In some embodiments, the average G/C content in the 3rd codon position in the ORF of the polynucleotide is at least 20%, at least 21%, at least 22%, at least 23%, at least 24%, at least 25%, at least 26%, at least 27%, at least 28%, at least 29%, or at least 30% higher than the average G/C content in the 3rd codon position in the corresponding wild-type ORF. In some embodiments, the increases in G and/or C content (absolute or relative) described herein can be conducted by replacing synonymous codons with low G, C, or G/C content with synonymous codons having higher G, C, or G/C content. In other embodiments, the increase in G and/or C content (absolute or relative) is conducted by replacing a codon ending with U with a synonymous codon ending with G or C.

In further embodiments, the ORF of the mRNA encoding a polypeptide includes less uracil pairs (UU) and/or uracil triplets (UUU) and/or uracil quadruplets (UUUU) than the corresponding wild-type nucleotide sequence encoding the polypeptide. In some embodiments, the ORF of the mRNA encoding a polypeptide of the disclosure includes no uracil pairs and/or uracil triplets and/or uracil quadruplets. In some embodiments, uracil pairs and/or uracil triplets and/or uracil quadruplets are reduced below a certain threshold, e.g., no more than 1, 2, 3, 4, 5, 6, 7, 8, 9, 10, 11, 12, 13, 14, 15, 16, 17, 18, 19, or 20 occurrences in the ORF of the mRNA encoding the polypeptide. In a particular embodiment, the ORF of the mRNA encoding the polypeptide of the disclosure contains less than 20, 19, 18, 17, 16, 15, 14, 13, 12, 11, 10, 9, 8, 7, 6, 5, 4, 3, 2, or 1 non-phenylalanine uracil pairs and/or triplets. In another embodiment, the ORF of the mRNA encoding the polypeptide contains no non-phenylalanine uracil pairs and/or triplets.

In further embodiments, the ORF of the mRNA encoding a polypeptide of the disclosure includes less uracil-rich clusters than the corresponding wild-type nucleotide sequence encoding the polypeptide. In some embodiments, the ORF of the mRNA encoding the polypeptide of the disclosure contains uracil-rich clusters that are shorter in length than corresponding uracil-rich clusters in the corresponding wild-type nucleotide sequence encoding the polypeptide.

In further embodiments, alternative lower frequency codons are employed. In some embodiment, the ORF of the polynucleotide further comprises at least one low-frequency codon. In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the codons in the polypeptide-encoding ORF of the mRNA are substituted with alternative codons, each alternative codon having a codon frequency lower than the codon frequency of the substituted codon in the synonymous codon set. The ORF may also have adjusted uracil content, as described above. In some embodiments, at least one codon in the ORF of the mRNA encoding the polypeptide is substituted with an alternative codon having a codon frequency lower than the codon frequency of the substituted codon in the synonymous codon set.

In some embodiments, the polynucleotide is an mRNA that comprises an ORF that encodes a polypeptide, wherein the uracil content of the ORF is between about 115% and about 135% of the theoretical minimum uracil content in the corresponding wild-type ORF, and wherein the uracil content in the ORF encoding the polypeptide is less than about 30% of the total nucleobase content in the ORF. In some embodiments, the ORF that encodes the polypeptide is further modified to increase G/C content of the ORF (absolute or relative) by at least about 40%, as compared to the corresponding wild-type ORF. In yet other embodiments, the ORF encoding the polypeptide contains less than 20 non-phenylalanine uracil pairs and/or triplets. In some embodiments, at least one codon in the ORF of the mRNA encoding the polypeptide is further substituted with an alternative codon having a codon frequency lower than the codon frequency of the substituted codon in the synonymous codon set.

In some embodiments, the expression of the polypeptide encoded by an mRNA comprising an ORF, wherein the uracil content of the ORF has been adjusted (e.g., the uracil content is between about 115% and about 135% of the theoretical minimum uracil content in the corresponding wild-type ORF) is increased by at least about 10-fold when compared to expression of the polypeptide from the corresponding wild-type mRNA. In some embodiments, the innate immune response induced by the mRNA including an open ORF wherein the uracil content has been adjusted (e.g., the uracil content of the ORF is between about 115% and about 135% of the theoretical minimum uracil content in the corresponding wild-type ORF) is reduced by at least about 10-fold when compared to expression of the polypeptide from the corresponding wild-type mRNA. In some embodiments, the mRNA with adjusted uracil content does not substantially induce an innate immune response of a mammalian cell into which the mRNA is introduced.

In some embodiments, the uracil content of the mRNA is adjusted as described herein, and a modified nucleoside is partially or completely substituted for the uracil remaining in the mRNA following adjustment. As a non-limiting example, the natural nucleotide uridine may be substituted with a modified nucleoside as described herein. In some embodiments, the modified nucleoside comprises pseudouridine (ψ). In some embodiments, the modified nucleoside comprises 1-methyl-pseudouridine (m1ψ). In some embodiments, the modified nucleoside comprises 1-methyl-pseudouridine (m1ψ) and 5-methyl-cytidine (m5C). In some embodiments, the modified nucleoside comprises 2-thiouridine (s2U). In some embodiments, the modified nucleoside comprises 2-thiouridine and 5-methyl-cytidine (m5C). In some embodiments, the modified nucleoside comprises 5-methoxy-uridine (mo5U). In some embodiments, the modified nucleoside comprises 5-methoxy-uridine (mo5U) and 5-methyl-cytidine (m5C). In some embodiments, the modified nucleoside comprises 2′-O-methyl uridine. In some embodiments, the modified nucleoside comprises 2′-O-methyl uridine and 5-methyl-cytidine (m5C). In some embodiments, the modified nucleoside comprises N6-methyl-adenosine (m6A). In some embodiments, the modified nucleoside comprises N6-methyl-adenosine (m6A) and 5-methyl-cytidine (m5C).

Chemical Modification of mRNA

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ϕ) pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τ m5U), 1-taurinomethyl-pseudouridine, 5-taurinomethyl-2-thio-uridine(τ m5s2U), 1-taurinomethyl-4-thio-pseudouridine, 5-methyl-uridine (m5U, i.e., having the nucleobase deoxythymine), 1-methyl-pseudouridine (m1) ϕ), 5-methyl-2-thio-uridine (m5s2U), 1-methyl-4-thio-pseudouridine (m1s4ϕ), 4-thio-1-methyl-pseudouridine, 3-methyl-pseudouridine (m3ϕ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine. 3-(3-amino-3-carboxypropyl)uridine (acp3U), 1-methyl-3-(3-amino-3-carboxypropyl)pseudouridine (acp3 ϕ), 5-(isopentenylaminomethyl)uridine (inm5U), 5-(isopentenylaminomethyl)-2-thio-uridine (inm5s2U), α-thio-uridine, 2′-O-methyl-uridine (Urn), 5,2′-O-dimethyl-uridine (m5Um), 2′-O-methyl-pseudouridine (ϕ m), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-0H-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include α-thio-adenosine. 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-0H-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1 G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 2′-O-ribosylguanosine (phosphate) (Gr(p)), 1-thio-guanosine, 06-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is pseudouridine (ϕ) N1-methylpseudouridine (m1ϕ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases.) In one embodiment, the modified nucleobase is N1-methylpseudouridine (m1ϕ) and the mRNA of the disclosure is fully modified with N1-methylpseudouridine (m1 ϕ). In some embodiments, N1-methylpseudouridine (m1ϕ) represents from 75-100% of the uracils in the mRNA. In some embodiments, N1-methylpseudouridine (m1 ϕ) represents 100% of the uracils in the mRNA.

In some embodiments, the modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified adenine. Exemplary nucleobases and nucleosides having a modified adenine include 7-deaza-adenine, 1-methyl-adenosine (m1A), 2-methyl-adenine (m2A), N6-methyl-adenosine (m6A). In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified guanine. Exemplary nucleobases and nucleosides having a modified guanine include inosine (I), 1-methyl-inosine (mil), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is 1-methyl-pseudouridine (m1 ϕ), 5-methoxy-uridine (mo5U), 5-methyl-cytidine (m5C), pseudouridine (ϕ) α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases.)

In some embodiments, the mRNA comprises pseudouridine (ϕ) In some embodiments, the mRNA comprises pseudouridine (ϕ) and 5-methyl-cytidine (m5C). In some embodiments, the mRNA comprises 1-methyl-pseudouridine (m1 ϕ). In some embodiments, the mRNA comprises 1-methyl-pseudouridine (m1 ϕ) and 5-methyl-cytidine (m5C). In some embodiments, the mRNA comprises 2-thiouridine (s2U). In some embodiments, the mRNA comprises 2-thiouridine and 5-methyl-cytidine (m5C). In some embodiments, the mRNA comprises 5-methoxy-uridine (mo5U). In some embodiments, the mRNA comprises 5-methoxy-uridine (mo5U) and 5-methyl-cytidine (m5C). In some embodiments, the mRNA comprises 2′-O-methyl uridine. In some embodiments, the mRNA comprises 2′-O-methyl uridine and 5-methyl-cytidine (m5C). In some embodiments, the mRNA comprises N6-methyl-adenosine (m6A). In some embodiments, the mRNA comprises N6-methyl-adenosine (m6A) and 5-methyl-cytidine (m5C).

In certain embodiments, an mRNA of the disclosure is uniformly modified (i.e., fully modified, modified through-out the entire sequence) for a particular modification. For example, an mRNA can be uniformly modified with N1-methylpseudouridine (m1 45) or 5-methyl-cytidine (m5C), meaning that all uridines or all cytosine nucleosides in the mRNA sequence are replaced with N1-methylpseudouridine (m1) or 5-methyl-cytidine (m5C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mmRNAs of the disclosure can include a combination of modifications to the sugar, the nucleobase, and/or the internucleoside linkage. These combinations can include any one or more modifications described herein.

In certain embodiments, the modified nucleosides may be partially or completely substituted for the natural nucleotides of the mRNAs of the disclosure. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, Calif.) and/or proprietary methods. In one embodiment, the mRNA sequence is optimized using optimization algorithms, e.g., to optimize expression in mammalian cells or enhance mRNA stability.

In certain embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998). Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

### Polypeptides of Interest

In some embodiments, an mRNA of the disclosure encodes a polypeptide of interest that is a therapeutic polypeptide. In some embodiments, an mRNA of the disclosure encodes a polypeptide of interest that is a full-length protein. In some embodiments, an mRNA of the disclosure encodes a polypeptide of interest that is a functional fragment of a full-length protein (e.g., a fragment of the full-length protein that includes one or more functional domains such that the functional activity of the full-length protein is retained). In some embodiments, an mRNA of the disclosure encodes a polypeptide of interest that is not naturally occurring. In some embodiments, an mRNA of the disclosure encodes a polypeptide of interest that is a modified protein comprised of one or more heterologous domains (e.g., a protein that is a fusion protein comprised of one or more domains that do not naturally occur in the protein such that the function of the protein is altered).

Exemplary types of proteins (e.g., infectious disease antigens, tumor cell antigens, soluble effector molecules, antibodies, enzymes, recruitment factors, transcription factors, membrane bound receptors or ligands) that are encoded by an mRNA of the disclosure are described in detail in the following subsections.

### Naturally Occurring Targets

In some embodiments, an mRNA of the disclosure encodes a polypeptide of interest that is a naturally occurring target. In some embodiments, an mRNA encodes a polypeptide of interest that when expressed, modulates a naturally occurring target (e.g., up- or down-regulates the activity of a naturally occurring target). In some embodiments, a naturally occurring target is a soluble protein that is secreted by a cell. In some embodiments, a naturally occurring target is a protein that is retained within a cell (e.g., an intracellular protein). In some embodiments, a naturally occurring target is a membrane-bound or transmembrane protein. Non-limiting examples of naturally occurring targets include soluble proteins (e.g., chemokines, cytokines, growth factors, antibodies, enzymes), intracellular proteins (e.g., intracellular signaling proteins, transcription factors, enzymes, structural proteins) and membrane-bound or transmembrane proteins (e.g., receptors, adhesion molecules, enzymes).

In some embodiments, an mRNA encodes a polypeptide of interest that when expressed is a full-length naturally occurring target (i.e., a full-length protein). In some embodiments, an mRNA encodes a polypeptide of interest that when expressed is a fragment or portion of a naturally occurring target (i.e., a fragment or portion of a full-length protein). For example, in one embodiment, the protein or fragment thereof can be an immunogenic polypeptide that can be used as a vaccine.

In some embodiments, an mRNA encodes a polypeptide that when expressed, modulates a naturally occurring target (e.g., by encoding the target itself or by functioning to modulate the activity of the target). In some embodiments, a polypeptide of interest acts in an autocrine fashion, i.e., the polypeptide exerts an effect directly on the cell into which the mRNA is delivered. In some embodiments, an encoded polypeptide of interest acts in a paracrine fashion, i.e., the encoded polypeptide exerts an indirect effect on a cell that is not the cell into which the mRNA is delivered (e.g., delivery of the mRNA into one type of cell results in secretion of a molecule that exerts an effects on another type of cell, such as a bystander cell). In some embodiments, an encoded polypeptide of interest acts in both an autocrine fashion and a paracrine fashion.

### Naturally Occurring Soluble Targets

In some embodiments, an mRNA encodes a polypeptide of interest that modulates the activity of a naturally occurring soluble target, for example by encoding the soluble target itself or by modulating the expression (e.g., transcription or translation) of the soluble target. Non-limiting examples of naturally occurring soluble targets include cytokines, chemokines, growth factors, enzymes, and antibodies.

In some embodiments, an mRNA encoding a polypeptide of interest stimulates (e.g., upregulates, enhances) the activation or activity of a cell type, for example in situations where stimulation of an immune response is desirable, such as in cancer therapy or treatment of an infectious disease (e.g., a viral, bacterial, fungal, protozoal or parasitic infection). In another embodiment, an mRNA encoding a polypeptide of interest inhibits (e.g., downregulates, reduces) the activation or activity of a cell, for example in situations where inhibition of an immune response is desirable, such as in autoimmune diseases, allergies and transplantation.

In some embodiments, an mRNA of the disclosure encodes a soluble target that is a cytokine or chemokine with desirable uses for stimulating or inhibiting immune responses, e.g., that is useful in treating cancer as described further below.

In some embodiments, an mRNA of the disclosure encodes a soluble target that is a cytokine that stimulates the activation or activity of a cell such as an immune cell.

In some embodiments, an mRNA of the disclosure encodes a chemokine or a chemokine receptor which is useful for stimulating the activation or activity of an immune cell. Chemokines have been demonstrated to control the trafficking of inflammatory cells (including granulocytes and monocytes/monocytes), as well as regulating the movement of a wide variety of immune cells (including lymphocytes, natural killer cells and dendritic cells). Thus, chemokines are involved both in regulating inflammatory responses and immune responses. Moreover, chemokines have been shown to have effects on the proliferative and invasive properties of cancer cells (for a review of chemokines, see e.g., Mukaida, N. et al. (2014) Mediators of inflammation, Article ID 170381, pg. 1-15).

In some embodiments, an mRNA of the disclosure encodes a recruitment factor which is useful to stimulate the homing, activation or activity of a cell. In one embodiment, the cell is an immune cell and the “recruitment factor” refers to a protein that promotes recruitment of an immune cell to a desired location (e.g., to a tumor site or an inflammatory site). For example, certain chemokines, chemokine receptors and cytokines have been shown to be involved in the recruitment of lymphocytes (see e.g., Oelkrug, C. and Ramage, J. M. (2014) Clin. Exp. Immunol. 178:1-8).

In some embodiments, an mRNA of the disclosure encodes an inhibitory cytokine or an antagonist of a stimulatory cytokine which is useful for inhibiting immune responses.

In some embodiments, an mRNA of the disclosure encodes a soluble target that is an antibody. As used herein, the term “antibody” refers to a whole antibody comprising two light chain polypeptides and two heavy chain polypeptides, or an antigen-binding fragment thereof. In some embodiments, a soluble target is a monoclonal antibody (e.g., full length monoclonal antibody) that displays a single binding specificity and affinity for a particular epitope. In some embodiments, a soluble target is an antigen binding fragment of a monoclonal antibody that retains the ability to bind a target antigen. Such fragments include, e.g., a single chain antibody, a single chain Fv fragment (scFv), an Fd fragment, an Fab fragment, an Fab′ fragment, or an F(ab′)₂ fragment.

In some embodiments, an mRNA of the disclosure encodes an antibody that recognizes a tumor antigen, against which a protective or a therapeutic immune response is desired, e.g., antigens expressed by a tumor cell. In some embodiments, a suitable antigen includes tumor associated antigens for the prevention or treatment of cancers.

In some embodiments, an mRNA of the disclosure encodes an antibody that recognizes an infectious disease antigen, against which protective or therapeutic immune responses are desired, e.g., an antigen present on a pathogen or infectious agent. In some embodiments, a suitable antigen includes an infectious disease associated antigen for the prevention or treatment of an infectious disease. Methods for identification of antigens on infectious disease agents that comprise protective epitopes (e.g., epitopes that when recognized by an antibody enable neutralization or blocking of infection caused by an infectious disease agent) are described in the art as detailed by Sharon, J. et al. (2013) Immunology 142:1-23. In some embodiments, an infectious disease antigen is present on a virus or on a bacterial cell.

In some embodiments, an mRNA of the disclosure encodes a soluble target that is a growth factor with desirable uses for modulating tissue healing and repair. A growth factor is a protein that stimulates the survival, growth, proliferation, migration or differentiation of cells, often for the purposes of promoting growth of lost tissue or enhancing the body's innate healing and repair mechanisms. In some embodiments, a growth factor is used to manipulate cells that include, but are not limited to, stromal cells (e.g., fibroblasts), immune cells, vascular cells (e.g., epithelial cells, platelets, pericytes), neural cells (e.g., astrocytes, neural stem cells, microglial cells), or bone cells (e.g., osteocyte, osteoblast, osteoclast, osteogenic cells).

In some embodiments, an mRNA of the disclosure encodes a soluble target that is an enzyme with desirable uses for modulating metabolism or growth in a subject. In some embodiments, an enzyme is administered to replace an endogenous enzyme that is absent or dysfunctional as described in Brady, R. et al, (2004) Lancet Neurol. 3:752. In some embodiments, an enzyme is used to treat a metabolic storage disease. A metabolic storage disease results from the systemic accumulation of metabolites due to the absence or dysfunction of an endogenous enzyme. Such metabolites include lipids, glycoproteins, and mucopolysaccharides. In some embodiments, an enzyme is used to reduce or eliminate the accumulation of monosaccharides, polysaccharides, glycoproteins, glycopeptides, glycolipids or lipids due to a metabolic storage disease.

### Naturally Occurring Intracellular Targets

In some embodiments, an mRNA of the disclosure encodes a polypeptide of interest that modulates the activity of a naturally occurring intracellular target, for example by encoding the intracellular target itself or by modulating the expression (e.g., transcription or translation) of the intracellular target in a cell. Non-limiting examples of naturally-occurring intracellular targets include transcription factors and cell signaling cascade molecules, including enzymes, that modulate cell growth, differentiation and communication. Additional examples include intracellular targets that regulate cell metabolism.

Suitable transcription factors and intracellular signaling cascade molecules for particular uses in stimulating or inhibiting cellular activity or responses are described in the art. In some embodiments, an mRNA of the disclosure encodes a transcription factor useful for stimulating the activation or activity of an immune cell. As used herein, a “transcription factor” refers to a DNA-binding protein that regulates the transcription of a gene. In some embodiments, an mRNA of the disclosure encodes a transcription factor that increases or polarizes an immune response.

In some embodiments, an mRNA of the disclosure encodes an intracellular adaptor protein (e.g., in a signal transduction pathway) useful for stimulating the activation or activity of a cell.

In some embodiments, an mRNA of the disclosure encodes an intracellular signaling protein useful for stimulating the activation or activity of a cell. In some embodiments, an mRNA of the disclosure encodes a tolerogenic transcription factor useful for inhibiting the activation or activity of an immune cell.

In some embodiments, an mRNA of the disclosure encodes an intracellular target that is a protein that is used to treat a metabolic disease or disorder.

In some embodiments, an mRNA of the disclosure encodes a polypeptide of interest that is a fully-functional mitochondrial protein (e.g., wild-type). In some embodiments, an mRNA of the disclosure encodes a mitochondrial protein encoded by mitochondrial DNA (e.g., a mitochondrial-encoded mitochondrial protein). In some embodiments, an mRNA of the disclosure encodes a mitochondrial protein encoded by nuclear DNA (e.g., a nuclear-encoded mitochondrial protein). In some embodiments, an mRNA of the disclosure is used to treat a mitochondrial disease resulting from a mutation in a mitochondrial protein. In some embodiments, translation of an mRNA encoding a mitochondrial protein provides sufficient quantity and/or activity of the protein to ameliorate a mitochondrial disease. In some embodiments, an mRNA encodes a polypeptide of interest that is a mitochondrial protein described in the MitoCarta2.0 mitochondrial protein inventory.

### Naturally Occurring Membrane Bound/Transmembrane Targets

In some embodiments, an mRNA of the disclosure encodes a polypeptide of interest that modulates the activity of a naturally-occurring membrane-bound/transmembrane target, for example by encoding the membrane-bound/transmembrane target itself or by modulating the expression (e.g., transcription or translation) of the membrane-bound/transmembrane target. Non-limiting examples of naturally-occurring membrane-bound/transmembrane targets include Cell surface receptors, growth factor receptors, costimulatory molecules, immune checkpoint molecules, homing receptors and HLA molecules.

In one embodiment, the membrane-bound/transmembrane targets are useful in stimulating or inhibiting immune responses are described herein. In some embodiments, an mRNA of the disclosure encodes a costimulatory factor that upregulates an immune response or is an antagonist of a costimulatory factor that downregulates an immune response. In some embodiments, an mRNA of the disclosure encodes an immune checkpoint protein that down-regulates immune cells (e.g., T cells). In some embodiments, an mRNA of the disclosure encodes a membrane-bound/transmembrane protein target that serves as a homing signal.

In some embodiments, an mRNA of the disclosure encodes a membrane-bound/transmembrane protein target that is an immune receptor, e.g., on a lymphocyte or monocyte.

### Modified Targets

In some embodiments, an mRNA of the disclosure encodes a polypeptide of interest that is a modified polypeptide. In some embodiments, an mRNA of the disclosure encodes a polypeptide of interest that modulates a modified target (e.g., up- or down-regulates the activity of a non-naturally-occurring target). Typically, an mRNA of the disclosure encodes a modified target. Alternatively, if a cell expresses a modified target, an mRNA-encoded polypeptide functions to modulate the activity of the modified target in the cell. In some embodiments, a non-naturally occurring target is a full-length target, such as a full-length modified protein. In some embodiments, a non-naturally occurring target is a fragment or portion of a non-naturally-occurring target, such as a fragment or portion of a modified protein. In some embodiments, an mRNA-encoded polypeptide when expressed acts in an autocrine fashion to modulate a modified target, i.e., exerts an effect directly on the cell into which the mRNA is delivered. Additionally or alternatively, an mRNA-encoded polypeptide when expressed acts in a paracrine fashion to modulates a modified target, i.e., exerts an effect indirectly on a cell other than the cell into which the mRNA is delivered (e.g., delivery of the mRNA into one type of cell results in secretion of a molecule that exerts effects on another type of cell, such as bystander cells). Non-limiting examples of modified proteins include modified soluble proteins (e.g., secreted proteins), modified intracellular proteins (e.g., intracellular signaling proteins, transcription factors) and modified membrane-bound or transmembrane proteins (e.g., receptors).

### Modified Soluble Targets

In some embodiments, an mRNA of the disclosure encodes a polypeptide of interest that modulates a modified soluble target (e.g., up- or down-regulates the activity of a non-naturally-occurring soluble target). In some embodiments, an mRNA of the disclosure encodes a polypeptide of interest that is a modified soluble target. In some embodiments, a modified soluble target is a soluble protein that has been modified to alter (e.g., increase or decrease) the half-life (e.g., serum half-life) of the protein. Modified soluble proteins with altered half-life include modified cytokines and chemokines. In some embodiments, a modified soluble target is a soluble protein that has been modified to incorporate a tether such that the soluble protein becomes tethered to a cell surface. Modified soluble proteins incorporating a tether include tethered cytokines and chemokines.

### Modified Intracellular Targets

In some embodiments, an mRNA of the disclosure encodes a polypeptide of interest that modulates a modified intracellular target (e.g., up- or down-regulates the activity of a non-naturally-occurring intracellular target). In some embodiments, an mRNA of the disclosure encodes polypeptide of interest that is a modified intracellular target. In some embodiments, a modified intracellular target is a constitutively active mutant of an intracellular protein, such as a constitutively active transcription factor or intracellular signaling molecule. In some embodiments, a modified intracellular target is a dominant negative mutant of an intracellular protein, such as a dominant negative mutant of a transcription factor or intracellular signaling molecule. In some embodiments, a modified intracellular target is an altered (e.g., mutated) enzyme, such as a mutant enzyme with increased or decreased activity within an intracellular signaling cascade.

Modified Membrane bound/Transmembrane Targets

In some embodiments, an mRNA of the disclosure encodes a polypeptide of interest that modulates a modified membrane-bound/transmembrane target (e.g., up- or down-regulates the activity of a non-naturally-occurring membrane-bound/transmembrane target). In some embodiments, an mRNA of the disclosure encodes a polypeptide of interest that is a modified membrane-bound/transmembrane target. In some embodiments, a modified membrane-bound/transmembrane target is a constitutively active mutant of a membrane-bound/transmembrane protein, such as a constitutively active cell surface receptor (i.e., activates intracellular signaling through the receptor without the need for ligand binding). In some embodiments, a modified membrane-bound/transmembrane target is a dominant negative mutant of a membrane-bound/transmembrane protein, such as a dominant negative mutant of a cell surface receptor. In some embodiments, a modified membrane-bound/transmembrane target is a molecule that inverts signaling of a cellular synapse (e.g., agonizes or antagonizes signaling of a receptor). In some embodiments, a modified membrane-bound/transmembrane target is a chimeric membrane-bound/transmembrane protein, such as a chimeric cell surface receptor.

As used herein, the term “chimeric antigen receptor (CAR)” refers to an artificial transmembrane protein receptor comprising an extracellular domain capable of binding to a predetermined CAR ligand or antigen, an intracellular segment comprising one or more cytoplasmic domains derived from signal transducing proteins different from the polypeptide from which the extracellular domain is derived, and a transmembrane domain.

### Characterization of Immune Cells

The present disclosure provides miRs that are differentially expressed in select types of immune cells or select cell states (e.g., activated vs. resting, cancerous vs. non-cancerous). The present disclosure is based, at least in part, on the discovery that mRNAs comprising at least one miR binding site for a miR that is differentially expressed in a target cell type compared to a plurality of non-target cell types has reduced expression of encoded polypeptide in the target cell type compared to non-target cell types.

In some embodiments, the immune cell is a human immune cell. In another embodiment, the immune cell is a primate immune cell. In another embodiment, the immune cell is a human or non-human primate immune cell.

In some embodiments, an immune cell type refers to a particular immune cell lineage, for example, myeloid cells (e.g, dendritic cells or DCs, monocytes, macrophages), lymphoid cells (e.g., B cells, T cells, NK cells), and granulocytes (e.g., neutrophils). Methods of characterizing immune cell lineage are known in the art. As a non-limiting example, immune cell lineage is characterized by expression of certain cell surface (e.g., extracellular or intracellular) markers. One method of measuring the presence of a specific markers is to label the marker with a fluorescently tagged reagent specific to the marker (e.g., a labeled antibody) and analyze the cells with a method of fluorescent measurement (e.g., flow cytometry, microscopy, visible spectroscopy).

In some embodiments, an immune cell type comprises myeloid cells. In some embodiments, a myeloid cell is a DC. In some embodiments, a DC is a myeloid DC. Non-limiting examples of markers used to characterize myeloid DCs include: CD1a, CD1b, CD1c, CD4, CD11b, CD11c, CD40, CD49b, CD80, CD83, CD86, CD197, CD205, CD207, CD209, CD273, CD304, DC Marker, F4/80, HLA-DR and MHC-II. In some embodiments, a DC is a plasmacytoid DC. Non-limiting examples of markers used to characterize plasmacytoid DCs include: CD1a, CD1b, CD1c, CD4, CD8, CD11b, CD11c, CD40, CD45R, CD49d, CD80, CD83, CD85g, CD123, CD197, CD273, CD303, CD304, DC marker, F4/80, HLA-DR, MHC-II, and Siglec H. In some embodiments, a DC is a lymphoid DC. Non-limiting examples of markers used to characterize lymphoid DCs include: BDCA-1, CD8, CD11b, CD11c, CD103. CD205 and MHC-II. In some embodiments, a DC is characterized by a combination of cell-surface markers. In some embodiments, a DC is characterized by the absence of non-DC lineage markers, including CD3, CD14, CD19, CD56 and CD66b.

In some embodiments, a myeloid cell is a monocyte. Non-limiting examples of markers used to characterize monocytes include: CCR2, CD11b, CD11c, CD14, CD16, CD43, CD86, CD115, CD172a, CD209, CX3CR1, F4/80, HLA-DR, Ly6C, and MHC-II. In some embodiments, a myeloid cell is a macrophage. Non-limiting examples of markers used to characterize macrophages include: CD11a, CD11b, CD11c, CD14, CD15, CD16/32, CD33, CD64, CD68, CD80, CD85k, CD86, CD105, CD107b, CD115, CD163, CD195, CD282, CD284, F4/80, GITRL, HLA-DR, Mac-2, and MHC-II. In some embodiments, a macrophage is differentiated to an M1 phenotype. Non-limiting examples of markers used to characterize a M1 phenotype include: CD86, CD80, CD68, MHC-II, IL-1R, TLR2, TLR4, and SOCS3. In some embodiments, a macrophage is differentiated to an M2 phenotype. Non-limiting examples of markers used to characterize a M2 phenotype include: CD163, MHC-II, SR, MMR/CD206, CD200R, TGM2, DecoyR, and IL-1R II. In some embodiments, a macrophage is an immature macrophage.

In some embodiments, an immune cell type comprises lymphoid cells. In some embodiments, a lymphoid cell is a helper T cell (e.g., CD4+ T cells). Non-limiting examples of markers used to characterize helper T cells include: CD3, CD4, CD26, CD94, CD119, CD183, CD191, CD195, CD254, CD366, IL-18R, lymphotoxin beta receptor, CCR8, CD184, CD193, CD194, CD197, CD278, and CD365. In some embodiments, a lymphoid cell is a regulatory T cell. Non-limiting examples of markers used to characterize regulatory T cells include: CD3, CD4, CD25, CD39, CD73, CD103, CD152, GARP, GITR, LAP, FOXP3, STATS, and Smad3.

In some embodiments, a lymphoid cell is a cytotoxic T cell (e.g., CD8+ T cell). Non-limiting examples of extracellular markers used to characterize cytotoxic T cells include: CD3, CD8, CD44, CD107a, CD178, CD253, granzyme A, granzyme B, TNF-α, IFN-γ, and perforin. In some embodiments, a lymphoid cell is a natural killer (NK) cell. Non-limiting examples of markers used to characterize NK cells include: CD11b, CD11c, CD16/32, CD49b, CD56, CD57, CD69, CD94, CD122, CD158, CD161, CD244, CD314, CD319, CD328, CD355, Ly49, and Ly108. In some embodiments, a lymphoid cell is a B cell. Non-limiting examples of markers used to characterize B cells include: CD2, CD5, CD19, CD20, CD21/CD35, CD22, CD23, CD40, CD45R, CD69, CD70, CD79a, CD79b, CD80, CD86, CD92, CD137, CD138, CD252, CD267, CD268, CD279, IgD, and IgM. In some embodiments, a B cell is differentiated to a B1 cell phenotype. In some embodiments, a B cell is differentiated to a B2 cell phenotype.

In some embodiments, an immune cell type comprises granulocytes. In some embodiments, a granulocyte is a neutrophil. Non-limiting examples of markers used to characterize neutrophils include: CD10. CD11b, CD11c, CD13, CD14, CD15, CD16/32, CD31, CD33, CD62L, CD64, CD66b, CD88, CD114, CXCR1, CXCR2, GR-1, JAML, and TLR2.

In one embodiment, the immune cell is a T cell (e.g., a CD3+ T cell, a CD4+ T cell, a CD8+ T cell or a CD4+CD25+CD127^{low} Treg cell). In one embodiment, the immune cell is a B cell (e.g., a CD19+ B cell). In one embodiments, the immune cell is a monocyte (e.g., a CD11b+CD14^{hi} monocyte). In one embodiment, the immune cell is a dendritic cell (e.g., a CD11c+CD11b-dendritic cell). In one embodiment, the immune cell is a macrophage (e.g., a CD11b+CD14^{low} macrophage). In one embodiment, the immune cell is a mature NK cell (e.g., a cD56^{intermediate} mature NK cell). In one embodiment, the immune cell is an immature NK cell (e.g., a CD56^{high} immature NK cell). In one embodiment, the immune cell is an NK T cell (e.g., a CD3+CD56+ NK T cell).

In some embodiments, an immune cell type refers to a cancerous immune cell. In some embodiments, a cancerous immune cell is an AML leukemia cell (e.g., a CD33+ AML cell).

In some embodiments, an immune cell type refers to an immune cell state. In some embodiments, an immune cell state refers to an activation state. In some embodiments, an activated cell is a CD3+ T cell. In some embodiments, an activated cell is a helper T cell (e.g., a CD3+CD4+ T cell). In some embodiments, an activated cell is a cytotoxic T cell (e.g., a CD3+CD8+ T cell). T cells can be activated using methods as described, for example, in U.S. Pat. Nos. 6,352,694; 6,534,055; 6,905.680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041 that are incorporated by reference herein. In some embodiments, a method of activating a T cell comprises treatment with phorbol 12-myristate 13-acetate (PMA). In some embodiments, a method of activating a T cell comprises treatment with ionomycin. In some embodiments, a method of activating a T cell comprises treatment OX40 ligand. In some embodiments, a method of treating with OX40 ligand comprises contacting the cells with an mRNA encoding OX40 ligand.

In some embodiments, an activated T cell is identified by expression of one or more markers (e.g., an extracellular marker, an intracellular marker). Non-limiting examples of markers expressed by activated T cells include: CD3, CD4, CD8. CD25, CD27, CD28, CD44, CD69, CD95, CD134, CD137, CD154, Ki-67 and KLRG1.

### Immune Cell Delivery LNPs

Immune cell delivery LNPs can be characterized in that they result in increased delivery of agents to immune cells as compared to a control LNP (e.g., an LNP lacking the immune cell delivery potentiating lipid). In particular, in one embodiment, immune cell delivery LNPs result in an increase (e.g., a 2-fold or more increase) in the percentage of LNPs associated with immune cells as compared to a control LNP or an increase (e.g., a 2-fold or more increase) in the percentage of immune cells expressing the agent carried by the LNP (e.g., expressing the protein encoded by the mRNA associated with/encapsulated by the LNP) as compared to a control LNP. In another embodiment, immune cell delivery LNPs result in increased binding to C1q and/or increased uptake of C1q-bound LNP into the immune cells (e.g., via opsonization) as compared to a control LNP (e.g., an LNP lacking the immune cell delivery potentiating lipid).

In another embodiment, immune cell delivery LNPs result in an increase in the delivery of an agent (e.g., a nucleic acid molecule) to immune cells as compared to a control LNP. In one embodiment, immune cell delivery LNPs result in an increase in the delivery of a nucleic acid molecule agent to T cells as compared to a control LNP. In one embodiment, immune cell delivery LNPs result in an increase in the delivery of a nucleic acid molecule agent to B cells as compared to a control LNP. In one embodiment, immune cell delivery LNPs result in an increase in the delivery of a nucleic acid molecule agent to B cells as compared to a control LNP. In one embodiment, immune cell delivery LNPs result in an increase in the delivery of a nucleic acid molecule agent to myeloid cells as compared to a control LNP.

In one embodiment, when the nucleic acid molecule is an mRNA, an increase in the delivery of a nucleic acid agent to immune cells can be measured by the ability of an LNP to effect at least about 2-fold greater expression of a protein molecule encoded by the mRNA in immune cells, (e.g., T cells) as compared to a control LNP.

Immune cell delivery LNPs comprise an (i) ionizable lipid; (ii) sterol or other structural lipid; (iii) a non-cationic helper lipid or phospholipid; a (iv) PEG lipid and (v) an agent (e.g., a nucleic acid molecule) encapsulated in and/or associated with the LNP, wherein one or more of (i) the ionizable lipid or (ii) the structural lipid or sterol in an immune cell delivery LNPs comprises an effective amount of an immune cell delivery potentiating lipid.

In another embodiment, an immune cell delivery lipid nanoparticle of the disclosure comprises:

(i) an ionizable lipid;

(ii) a sterol or other structural lipid;

(iii) a non-cationic helper lipid or phospholipid;

(iv) an agent for delivery to an immune cell, and

(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to an immune cell. In one embodiment, enhanced delivery is relative to a lipid nanoparticle lacking the immune cell delivery potentiating lipid. In another embodiment, the enhanced delivery is relative to a suitable control.

In another embodiment, an immune cell delivery lipid nanoparticle of the disclosure comprises:

(i) an ionizable lipid;

(ii) a sterol or other structural lipid;

(iii) a non-cationic helper lipid or phospholipid;

(iv) an agent for delivery to an immune cell, and

(v) optionally, a PEG-lipid

wherein one or more of (i) the ionizable lipid or (ii) the sterol or other structural lipid or (iii) the non-cationic helper lipid or phospholipid or (v) the PEG lipid is a C1q binding lipid that binds to C1q or promotes (e.g., increases, stimulates, enhances) the binding of the LNP to C1q, as compared to a control LNP lacking the C1q binding lipid.

In another embodiment, an immune cell delivery lipid nanoparticle of the disclosure comprises:

(i) an ionizable lipid;

(ii) a sterol or other structural lipid;

(iii) a non-cationic helper lipid or phospholipid;

(iv) an agent for delivery to an immune cell, and

(v) optionally, a PEG-lipid

wherein one or more of (i) the ionizable lipid or (ii) the sterol or other structural lipid binds to C1q or promotes (e.g., increases, stimulates, enhances) the binding of the LNP to C1q, as compared to a control LNP (e.g., an LNP lacking (i) the ionizable lipid or (ii) the sterol or other structural lipid).

In another aspect, the disclosure provides a method of screening for an immune cell delivery lipid, the method comprising contacting a test LNP comprising a test immune cell delivery lipid with C1q, and measuring binding to C1q, wherein a test immune cell delivery lipid is selected as an immune cell delivery lipid when it binds to C1q or promotes (e.g., increases, stimulates, enhances) the binding of the LNP comprising it to C1q.

### Lipid Content of LNPs

As set forth above, with respect to lipids, immune cell delivery LNPs comprise an (i) ionizable lipid; (ii) sterol or other structural lipid; (iii) a non-cationic helper lipid or phospholipid; a (iv) PEG lipid, wherein one or more of (i) the ionizable lipid or (ii) the structural lipid or sterol in an immune cell delivery LNPs comprises an effective amount of an immune cell delivery potentiating lipid. These categories of lipids are set forth in more detail below.

(i) Ionizable Lipids

The lipid nanoparticles of the present disclosure include one or more ionizable lipids. In certain embodiments, the ionizable lipids of the disclosure comprise a central amine moiety and at least one biodegradable group. The ionizable lipids described herein may be advantageously used in lipid nanoparticles of the disclosure for the delivery of nucleic acid molecules to mammalian cells or organs. The structures of ionizable lipids set forth below include the prefix I to distinguish them from other lipids of the invention.

In a first aspect of the invention, the compounds described herein are of Formula (I I):

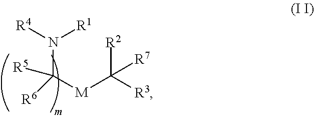

or their N-oxides, or salts or isomers thereof, wherein:

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═N R⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;

each R⁵ is independently selected from the group consisting of OH, C₁₋₃ alkyl, C2-3 alkenyl, and H;

each R⁶ is independently selected from the group consisting of OH, C₁₋₃ alkyl, C2-3 alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

R⁸ is selected from the group consisting of C₃₋₆ carbocycle and heterocycle;

R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

R¹⁰ is selected from the group consisting of H, OH, C₁₋₃ alkyl, and C₂₋₃ alkenyl; each R is independently selected from the group consisting of C₁₋₃ alkyl, C₂₋₃ alkenyl, (CH₂)_{q}OR*, and H,

and each q is independently selected from 1, 2, and 3;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, C₁, Br, and I; and

m is selected from 5, 6, 7, 8, 9, 10, 11, 12, and 13; and wherein when R⁴ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

Another aspect the disclosure relates to compounds of Formula (III):

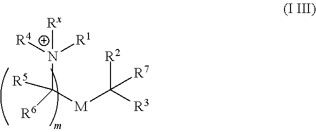

### or its N-oxide,

or a salt or isomer thereof, wherein

or a salt or isomer thereof, wherein

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX3, —CX2H, —CXH₂, —CN, —N(R)₂, —C(O)N(R)₂, —N(R)C(O)R, —N(R)S(O)₂R, —N(R)C(O)N(R)₂, —N(R)C(S)N(R)₂, N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;

R^{x} is selected from the group consisting of C₁₋₆ alkyl, C₂₋₆ alkenyl, —(CH₂)ᵥOH, and —(CH₂)ᵥN(R)₂,

wherein v is selected from 1, 2, 3, 4, 5, and 6;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

R⁸ is selected from the group consisting of C₃₋₆ carbocycle and heterocycle;

R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

R¹⁰ is selected from the group consisting of H, OH, C₁₋₃ alkyl, and C₂₋₃ alkenyl; each R is independently selected from the group consisting of C₁₋₃ alkyl, C₂₋₃ alkenyl, (CH₂)_{q}OR*, and H,

and each q is independently selected from 1, 2, and 3;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13.

In certain embodiments, a subset of compounds of Formula (I) includes those of Formula (IA):

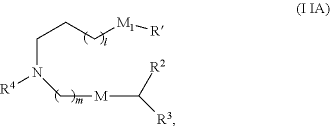

or its N-oxide, or a salt or isomer thereof, wherein 1 is selected from 1, 2, 3, 4, and 5; m is selected from 5, 6, 7, 8, and 9; M₁ is a bond or M′; R⁴ is hydrogen, unsubstituted C₁₋₃ alkyl, —(CH₂)ₒC(R¹⁰)₂(CH₂)ₙ₋ₒQ, or —(CH₂)ₙQ, in which Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR,

heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group, and R² and R³ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IB):

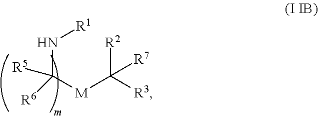

or its N-oxide, or a salt or isomer thereof in which all variables are as defined herein. For example, m is selected from 5, 6, 7, 8, and 9; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, m is 5, 7, or 9. In certain embodiments, a subset of compounds of Formula (I) includes those of Formula (II):

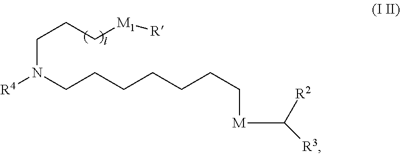

or its N-oxide, or a salt or isomer thereof, wherein 1 is selected from 1, 2, 3, 4, and 5; M₁ is a bond or M′; R⁴ is hydrogen, unsubstituted C₁₋₃ alkyl, —(CH₂)ₒC(R¹⁰ 2(CH₂)ₙ₋ₒQ, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR,

heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl.

Another aspect of the disclosure relates to compounds of Formula (I VI):

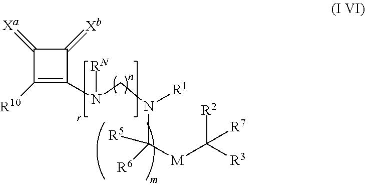

### or its N-oxide,

or a salt or isomer thereof, wherein

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

each R is independently selected from the group consisting of H, C₁₋₃ alkyl, and C₂₋₃ alkenyl;

R^{N} is H, or C₁₋₃ alkyl;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, C₁, Br, and I;

X^{a} and X^{b} are each independently O or S;

R¹⁰ is selected from the group consisting of H, halo, —OH, R, —N(R)₂, —CN, —N3, —C(O)OH, —C(O)OR, —OC(O)R, —OR, —SR, —S(O)R, —S(O)OR, —S(O)₂OR, —NO₂, —S(O)₂N(R)₂, —N(R)S(O)₂R, —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, a carbocycle, a heterocycle, aryl and heteroaryl;

m is selected from 5, 6, 7, 8, 9, 10, 11, 12, and 13;

n is selected from 1, 2, 3, 4, 5, 6, 7, 8, 9, and 10;

r is 0 or 1;

t¹ is selected from 1, 2, 3, 4, and 5;

p¹ is selected from 1, 2, 3, 4, and 5;

q¹ is selected from 1, 2, 3, 4, and 5; and

s¹ is selected from 1, 2, 3, 4, and 5.

In one embodiment, a subset of compounds of Formula (VI) includes those of Formula (VI-a):

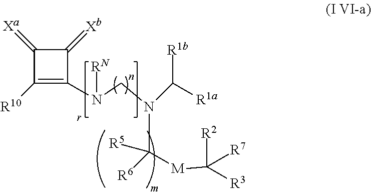

### or its N-oxide,

or a salt or isomer thereof, wherein

R^{1a} and R^{1b} are independently selected from the group consisting of C₁₋₁₄ alkyl and C₂₋₁₄ alkenyl; and

R² and R³ are independently selected from the group consisting of C₁₋₁₄ alkyl, C₂₋₁₄ alkenyl, —R*YR″, —YR″, and —R*OR″, or R² and R³, together with the atom to which they are attached, form a heterocycle or carbocycle.

In another embodiment, a subset of compounds of Formula (VI) includes those of Formula (VII):

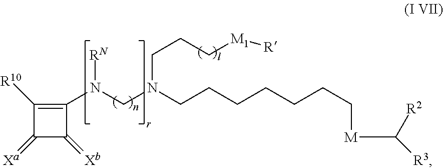

or its N-oxide, or a salt or isomer thereof, wherein

1 is selected from 1, 2, 3, 4, and 5;

M1 is a bond or M′; and

R² and R³ are independently selected from the group consisting of H, C₁₋₁₄ alkyl, and C₂-14 alkenyl.

In another embodiment, a subset of compounds of Formula (I VI) includes those of Formula (I VIII):

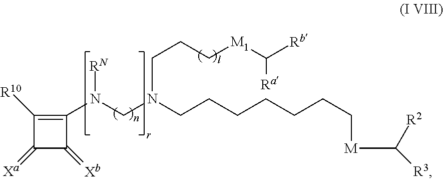

or its N-oxide, or a salt or isomer thereof, wherein

1 is selected from 1, 2, 3, 4, and 5;

M₁ is a bond or M′; and

R^{a′} and R^{b′} are independently selected from the group consisting of C₁₋₁₄ alkyl and C₂₋₁₄ alkenyl; and

R² and R³ are independently selected from the group consisting of C₁₋₁₄ alkyl, and C₂₋₁₄ alkenyl.

The compounds of any one of formula (I I), (I IA), (I VI), (I VI-a), (I VII) or (I VIII) include one or more of the following features when applicable.

In some embodiments, M₁ is M′.

In some embodiments, M and M′ are independently —C(O)O— or —OC(O)—.

In some embodiments, at least one of M and M′ is —C(O)O— or —OC(O)—.

In certain embodiments, at least one of M and M′ is —OC(O)—.

In certain embodiments, M is —OC(O)— and M′ is —C(O)O—. In some embodiments, M is —C(O)O— and M′ is —OC(O)—. In certain embodiments, M and M′ are each —OC(O)—. In some embodiments, M and M′ are each —C(O)O—.

In certain embodiments, at least one of M and M′ is —OC(O)-M″-C(O)O—.

In some embodiments, M and M′ are independently —S—S—.

In some embodiments, at least one of M and M′ is —S—S.

In some embodiments, one of M and M′ is —C(O)O— or —OC(O)— and the other is —S—S—. For example, M is —C(O)O— or —OC(O)— and M′ is —S—S— or M′ is —C(O)O—, or —OC(O)— and M is —S—S—.

In some embodiments, one of M and M′ is —OC(O)-M″-C(O)O—, in which M″ is a bond, C₁₋₁₃ alkyl or C₂₋₁₃ alkenyl. In other embodiments, M″ is C₁₋₆ alkyl or C₂₋₆ alkenyl. In certain embodiments, M″ is C₁₋₄ alkyl or C₂₋₄ alkenyl. For example, in some embodiments, M″ is C₁ alkyl. For example, in some embodiments, M″ is C₂ alkyl. For example, in some embodiments, M″ is C₃ alkyl. For example, in some embodiments, M″ is C₄ alkyl. For example, in some embodiments, M″ is C₂ alkenyl. For example, in some embodiments, M″ is C₃ alkenyl. For example, in some embodiments, M″ is C₄ alkenyl.

In some embodiments, 1 is 1, 3, or 5.

In some embodiments, R⁴ is hydrogen.

In some embodiments, R⁴ is not hydrogen.

In some embodiments, R⁴ is unsubstituted methyl or —(CH₂)ₙQ, in which Q is OH, —NHC(S)N(R)₂, —NHC(O)N(R)₂, —N(R)C(O)R, or —N(R)S(O)₂R.

In some embodiments, Q is OH.

In some embodiments, Q is —NHC(S)N(R)₂.

In some embodiments, Q is —NHC(O)N(R)₂.

In some embodiments, Q is —N(R)C(O)R.

In some embodiments, Q is —N(R)S(O)₂R.

In some embodiments, Q is —O(CH₂)ₙN(R)₂.

In some embodiments, Q is —O(CH₂)ₙOR.

In some embodiments, Q is —N(R)R⁸.

In some embodiments, Q is —NHC(═NR⁹)N(R)₂.

In some embodiments, Q is —NHC(═CHR⁹)N(R)₂.

In some embodiments, Q is —OC(O)N(R)₂.

In some embodiments, Q is —N(R)C(O)OR.

In some embodiments, n is 2.

In some embodiments, n is 3.

In some embodiments, n is 4.

In some embodiments, M₁ is absent.

In some embodiments, at least one R⁵ is hydroxyl. For example, one R⁵ is hydroxyl.

In some embodiments, at least one R⁶ is hydroxyl. For example, one R⁶ is hydroxyl.

In some embodiments one of R⁵ and R⁶ is hydroxyl. For example, one R⁵ is hydroxyl and each R⁶ is hydrogen. For example, one R⁶ is hydroxyl and each R⁵ is hydrogen.

In some embodiments, R^{x} is C₁₋₆ alkyl. In some embodiments, R^{x} is C₁₋₃ alkyl. For example, R^{x} is methyl. For example, R^{x} is ethyl. For example, R^{x} is propyl.

In some embodiments, R^{x} is —(CH₂)ᵥOH and, v is 1, 2 or 3. For example, R^{x} is methanoyl. For example, R^{x} is ethanoyl. For example, R^{x} is propanoyl.

In some embodiments, R^{x} is —(CH₂)ᵥN(R)₂, v is 1, 2 or 3 and each R is H or methyl. For example, R^{x} is methanamino, methylmethanamino, or dimethylmethanamino. For example, R^{x} is aminomethanyl, methylaminomethanyl, or dimethylaminomethanyl. For example, R^{x} is aminoethanyl, methylaminoethanyl, or dimethylaminoethanyl. For example, R^{x} is aminopropanyl, methylaminopropanyl, or dimethylaminopropanyl.

In some embodiments, R′ is C₁₋₁₈ alkyl, C₂₋₁₈ alkenyl, —R*YR″, or —YR″.

In some embodiments, R² and R³ are independently C₃₋₁₄ alkyl or C₃₋₁₄ alkenyl.

In some embodiments, R^{1b} is C₁₋₁₄ alkyl. In some embodiments, R^{1b} is C₂₋₁₄ alkyl. In some embodiments, R^{1b} is C₃₋₁₄ alkyl. In some embodiments, R^{1b} is C₁₋₈ alkyl. In some embodiments, R^{1b} is C₁₋₅ alkyl. In some embodiments, R^{1b} is C₁₋₃ alkyl. In some embodiments, R^{1b} is selected from C₁ alkyl, C₂ alkyl, C₃ alkyl, C₄ alkyl, and C₅ alkyl. For example, in some embodiments, R^{1b} is C₁ alkyl. For example, in some embodiments, R^{1b} is C₂ alkyl. For example, in some embodiments, R^{1b} is C₃ alkyl. For example, in some embodiments, R^{1b} is C₄ alkyl. For example, in some embodiments, R^{1b} is C₅ alkyl.

In some embodiments, R¹ is different from —(CHR⁵R⁶)ₘ-M-CR²R³R⁷.

In some embodiments, —CHR^{1a}R^{1b} is different from —(CHR⁵R⁶)ₘ-M-CR²R³R⁷.

In some embodiments, R⁷ is H. In some embodiments, R⁷ is selected from C₁₋₃ alkyl. For example, in some embodiments, R⁷ is C₁ alkyl. For example, in some embodiments, R⁷ is C₂ alkyl. For example, in some embodiments, R⁷ is C₃ alkyl. In some embodiments, R⁷ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and C₁₈ alkenyl.

In some embodiments, R^{b′} is C₁₋₁₄ alkyl. In some embodiments, R^{b′} is C₂₋₁₄ alkyl. In some embodiments, R^{b′} is C₃₋₁₄ alkyl. In some embodiments, R^{b′} is C₁₋₈ alkyl. In some embodiments, R^{b′} is C₁₋₅ alkyl. In some embodiments, R^{b′} is C₁₋₃ alkyl. In some embodiments, R^{b′} is selected from C₁ alkyl, C₂ alkyl, C₃ alkyl, C₄ alkyl and C₅ alkyl. For example, in some embodiments, R^{b′} is C₁ alkyl. For example, in some embodiments, R^{b′} is C₂ alkyl. For example, some embodiments, R^{b′} is C₃ alkyl. For example, some embodiments, R^{b′} is C₄ alkyl.

In one embodiment, the compounds of Formula (I) are of Formula (IIa):

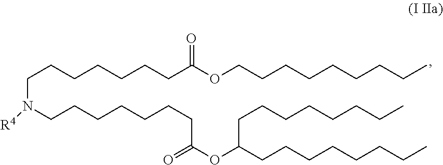

or their N-oxides, or salts or isomers thereof, wherein R⁴ is as described herein.

In another embodiment, the compounds of Formula (I) are of Formula (IIb):

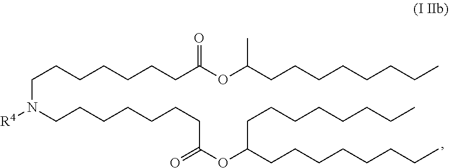

or their N-oxides, or salts or isomers thereof, wherein R⁴ is as described herein.

In another embodiment, the compounds of Formula (I) are of Formula (IIc) or (IIe):

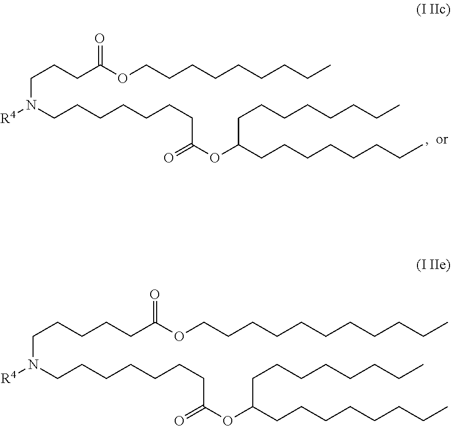

or their N-oxides, or salts or isomers thereof, wherein R⁴ is as described herein.

In another embodiment, the compounds of Formula (I I) are of Formula (I IIf):

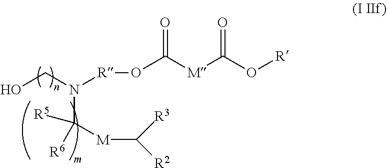

or their N-oxides, or salts or isomers thereof,

wherein M is —C(O)O— or —OC(O)—, M″ is C₁₋₆ alkyl or C₂₋₆ alkenyl, R² and R³ are independently selected from the group consisting of C₅₋₁₄ alkyl and C₅₋₁₄ alkenyl, and n is selected from 2, 3, and 4.

In a further embodiment, the compounds of Formula (I I) are of Formula (IId):

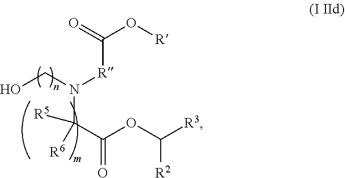

or their N-oxides, or salts or isomers thereof, wherein n is 2, 3, or 4; and m, R′, R″, and R² through R₆ are as described herein. For example, each of R² and R³ may be independently selected from the group consisting of C₅₋₁₄ alkyl and C₅₋₁₄ alkenyl.

In a further embodiment, the compounds of Formula (I) are of Formula (IIg):

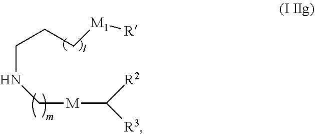

or their N-oxides, or salts or isomers thereof, wherein 1 is selected from 1, 2, 3, 4, and 5; m is selected from 5, 6, 7, 8, and 9; M₁ is a bond or M′; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R² and R³ are independently selected from the group consisting of C₅₋₁₄ alkyl and C₅₋₁₄ alkenyl.

In another embodiment, a subset of compounds of Formula (I VI) includes those of Formula (I VIIa):

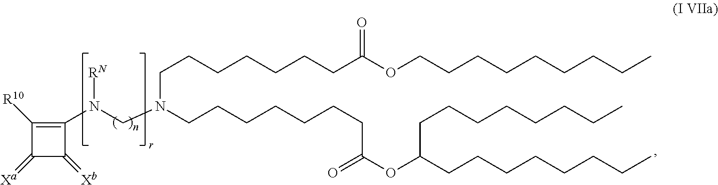

or its N-oxide, or a salt or isomer thereof.

In another embodiment, a subset of compounds of Formula (I VI) includes those of Formula (I VIIIa):

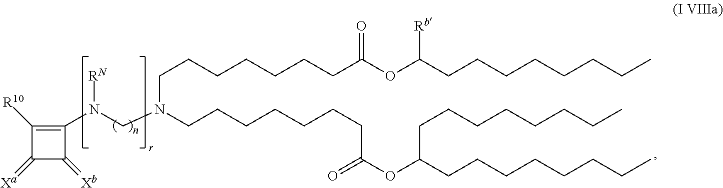

or its N-oxide, or a salt or isomer thereof.

In another embodiment, a subset of compounds of Formula (I VI) includes those of Formula (I VIIIb):

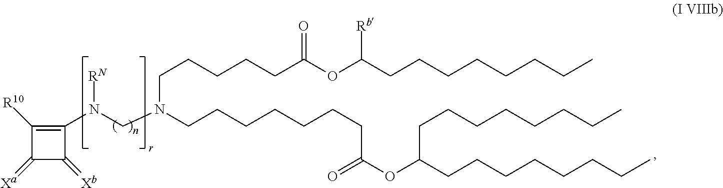

or its N-oxide, or a salt or isomer thereof.

In another embodiment, a subset of compounds of Formula (I VI) includes those of Formula (I VIIb-1):

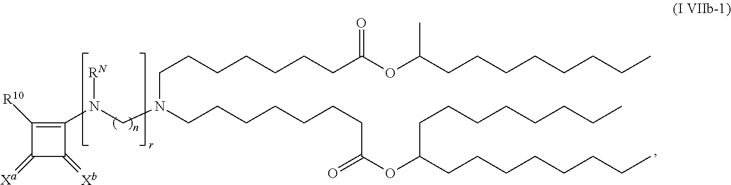

or its N-oxide, or a salt or isomer thereof.

In another embodiment, a subset of compounds of Formula (I VI) includes those of Formula (I VIIb-2):

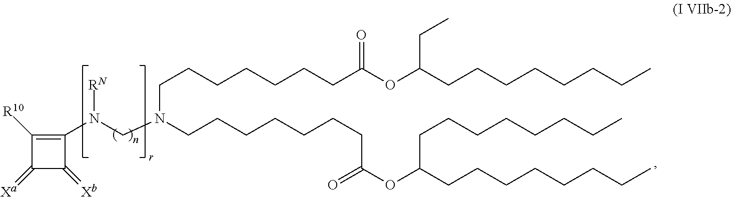

or its N-oxide, or a salt or isomer thereof.

In another embodiment, a subset of compounds of Formula (I VI) includes those of Formula (I VIIb-3):

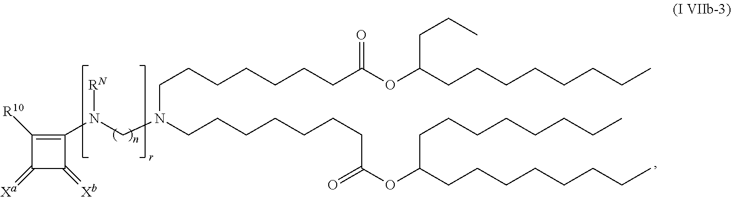

or its N-oxide, or a salt or isomer thereof. In another embodiment, a subset of compounds of Formula (VI) includes those of Formula (VIIc):

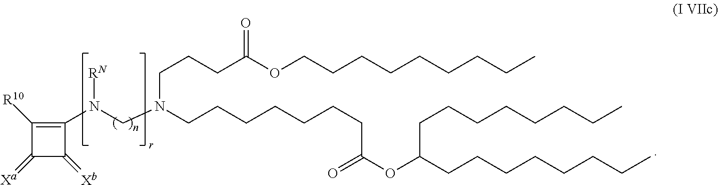

In another embodiment, a subset of compounds of Formula (I VI) includes those of Formula (VIId):

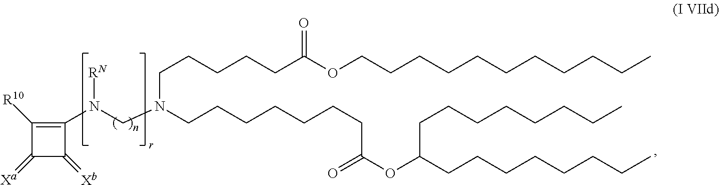

or its N-oxide, or a salt or isomer thereof.

In another embodiment, a subset of compounds of Formula (I VI) includes those of Formula (I VIIIc):

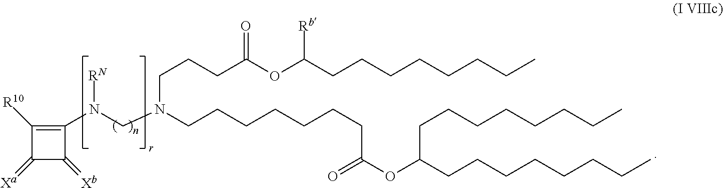

In another embodiment, a subset of compounds of Formula I VI) includes those of Formula (I VIIId):

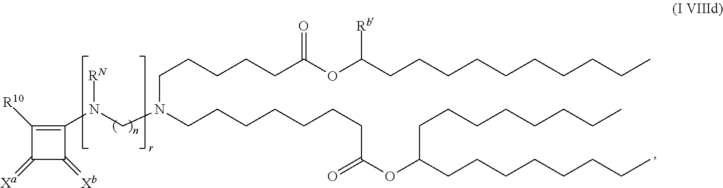

or its N-oxide, or a salt or isomer thereof.

The compounds of any one of formulae (I I), (I IA), (I IB), (I II), (I IIa), (I IIb), (I IIc), (I IId), (I IIe), (I IIf), (I IIg), I (III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), or (I VIIId) include one or more of the following features when applicable.

In some embodiments, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, 5- to 14-membered aromatic or non-aromatic heterocycle having one or more heteroatoms selected from N, O, S, and P, —OR, —O(CH₂)ₙN(R)₂, —C(O)OR, —OC(O)R, —CX3, —CX2H, —CXH₂, —CN, —N(R)₂, —N(R)S(O)₂R⁸, —C(O)N(R)₂, —N(R)C(O)R, —N(R)S(O)₂R, —N(R)C(O)N(R)₂, —N(R)C(S)N(R)₂, and —C(R)N(R)₂C(O)OR, each o is independently selected from 1, 2, 3, and 4, and each n is independently selected from 1, 2, 3, 4, and 5.

In another embodiment, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX3, —CX2H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, and a 5- to 14-membered heterocycloalkyl having one or more heteroatoms selected from N, O, and S which is substituted with one or more substituents selected from oxo (═O), OH, amino, and C₁₋₃ alkyl, each o is independently selected from 1, 2, 3, and 4, and each n is independently selected from 1, 2, 3, 4, and 5.

In another embodiment, R⁴ is selected from the group consisting of a C₃-6 carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, each o is independently selected from 1, 2, 3, and 4, and each n is independently selected from 1, 2, 3, 4, and 5; and when Q is a 5- to 14-membered heterocycle and (i) R⁴ is —(CH₂)ₙQ in which n is 1 or 2, or (ii) R⁴ is —(CH₂)ₙCHQR in which n is 1, or (iii) R⁴ is —CHQR, and —CQ(R)₂, then Q is either a 5- to 14-membered heteroaryl or 8- to 14-membered heterocycloalkyl.

In another embodiment, R⁴ is selected from the group consisting of a C₃-6 carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX3, —CX2H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, each o is independently selected from 1, 2, 3, and 4, and each n is independently selected from 1, 2, 3, 4, and 5.

In another embodiment, R⁴ is —(CH₂)ₙQ, where Q is —N(R)S(O)₂R⁸ and n is selected from 1, 2, 3, 4, and 5. In a further embodiment, R⁴ is —(CH₂)ₙQ, where Q is —N(R)S(O)₂R⁸, in which R⁸ is a C₃₋₆ carbocycle such as C₃₋₆ cycloalkyl, and n is selected from 1, 2, 3, 4, and 5. For example, R⁴ is —(CH₂)₃NHS(O)₂R⁸ and R⁸ is cyclopropyl.

In another embodiment, R⁴ is —(CH₂)ₒC(R¹⁰)₂(CH₂)ₙ₋ₒQ, where Q is —N(R)C(O)R, n is selected from 1, 2, 3, 4, and 5, and o is selected from 1, 2, 3, and 4. In a further embodiment, R⁴ is —(CH₂)ₒC(R¹⁰)₂(CH₂)ₙ₋ₒQ, where Q is —N(R)C(O)R, wherein R is C₁-C₃ alkyl and n is selected from 1, 2, 3, 4, and 5, and o is selected from 1, 2, 3, and 4. In a another embodiment. R⁴ is is —(CH₂)ₒC(R¹⁰)₂(CH₂)ₙ₋ₒQ, where Q is —N(R)C(O)R, wherein R is C₁-C₃ alkyl, n is 3, and o is 1. In some embodiments, R¹⁰ is H, OH, C₁₋₃ alkyl, or C₂₋₃ alkenyl. For example, R⁴ is 3-acetamido-2,2-dimethylpropyl.

In some embodiments, one R¹⁰ is H and one R¹⁰ is C₁₋₃ alkyl or C₂₋₃ alkenyl. In another embodiment, each R¹⁰ is C₁₋₃ alkyl or C₂₋₃ alkenyl. In another embodiment, each R′° is C₁₋₃ alkyl (e.g. methyl, ethyl or propyl). For example, one R¹⁰ is methyl and one R¹⁰ is ethyl or propyl. For example, one R¹⁰ is ethyl and one R¹⁰ is methyl or propyl. For example, one R¹⁰ is propyl and one R¹⁰ is methyl or ethyl. For example, each R¹⁰ is methyl. For example, each R′° is ethyl. For example, each R¹⁰ is propyl.

In some embodiments, one R¹⁰ is H and one R¹⁰ is OH. In another embodiment, each R¹⁰ is OH.

In another embodiment, R⁴ is unsubstituted C₁₋₄ alkyl, e.g., unsubstituted methyl.

In another embodiment, R⁴ is hydrogen.

In certain embodiments, the disclosure provides a compound having the Formula (I), wherein R⁴ is —(CH₂)ₙQ or —(CH₂)ₙCHQR, where Q is —N(R)₂, and n is selected from 3, 4, and 5.

In certain embodiments, the disclosure provides a compound having the Formula (I), wherein R⁴ is selected from the group consisting of —(CH₂)ₙQ, —(CH₂)ₙCHQR, —CHQR, and —CQ(R)₂, where Q is —N(R)₂, and n is selected from 1, 2, 3, 4, and 5.

In certain embodiments, the disclosure provides a compound having the Formula (I), wherein R² and R³ are independently selected from the group consisting of C₂₋₁₄ alkyl, C₂₋₁₄ alkenyl, —R*YR″, —YR″, and —R*OR″, or R² and R³, together with the atom to which they are attached, form a heterocycle or carbocycle, and R⁴ is —(CH₂)ₙQ or —(CH₂)ₙCHQR, where Q is —N(R)₂, and n is selected from 3, 4, and 5.

In certain embodiments, R² and R³ are independently selected from the group consisting of C₂₋₁₄ alkyl, C₂₋₁₄ alkenyl, —R*YR″, —YR″, and —R*OR″, or R² and R³, together with the atom to which they are attached, form a heterocycle or carbocycle. In some embodiments, R² and R³ are independently selected from the group consisting of C₂₋₁₄ alkyl, and C₂₋₁₄ alkenyl. In some embodiments, R² and R³ are independently selected from the group consisting of —R*YR″, —YR″, and —R*OR″. In some embodiments, R² and R³ together with the atom to which they are attached, form a heterocycle or carbocycle.

In some embodiments, R¹ is selected from the group consisting of C₅₋₂₀ alkyl and C₅₋₂₀ alkenyl. In some embodiments, R¹ is C₅₋₂₀ alkyl substituted with hydroxyl.

In other embodiments, R¹ is selected from the group consisting of —R*YR″, —YR″, and —R″M′R′.

In certain embodiments, R¹ is selected from —R*YR″ and —YR″. In some embodiments, Y is a cyclopropyl group. In some embodiments, R* is C₈ alkyl or C₈ alkenyl. In certain embodiments, R″ is C₃₋₁₂ alkyl. For example, R″ may be C₃ alkyl. For example, R″ may be C₄₋₈ alkyl (e.g., C₄, C₅, C₆, C₇, or C₈ alkyl).

In some embodiments, R is (CH₂)_{q}OR*, q is selected from 1, 2, and 3, and R* is C₁₋₁₉ alkyl substituted with one or more substituents selected from the group consisting of amino, C₁-C₆ alkylamino, and C₁-C₆ dialkylamino. For example, R is (CH₂)_{q}OR*, q is selected from 1, 2, and 3 and R* is C₁₋₁₂ alkyl substituted with C₁-C₆dialkylamino. For example, R is (CH₂)_{q}OR*, q is selected from 1, 2, and 3 and R* is C₁₋₃ alkyl substituted with C₁-C₆dialkylamino. For example, R is (CH₂)_{q}OR*, q is selected from 1, 2, and 3 and R* is C₁₋₃ alkyl substituted with dimethylamino (e.g., dimethylaminoethanyl).

In some embodiments, R¹ is C₅₋₂₀ alkyl. In some embodiments, R¹ is C₆ alkyl. In some embodiments, R¹ is C₈ alkyl. In other embodiments, R¹ is C₉ alkyl. In certain embodiments, R¹ is C₁₄ alkyl. In other embodiments, R¹ is C₁₈ alkyl.

In some embodiments, R¹ is C₂₁₋₃₀ alkyl. In some embodiments, R¹ is C₂₆ alkyl. In some embodiments, R¹ is C₂₈ alkyl. In certain embodiments, R¹ is

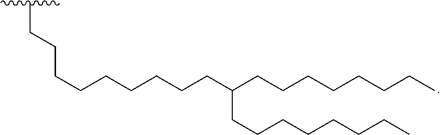 [SMILES: *CCCCCCCCCC(CCCCCCCC)CCCCCCCC]

In some embodiments, R¹ is C₅₋₂₀ alkenyl. In certain embodiments, R¹ is C₁₈ alkenyl. In some embodiments, R¹ is linoleyl.

In certain embodiments, R¹ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl, or heptadeca-9-yl). In certain embodiments, R¹ is

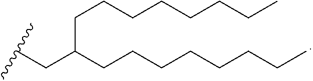 [SMILES: *CC(CCCCCCCC)CCCCCCCC]

In certain embodiments, R¹ is unsubstituted C₅₋₂₀ alkyl or C₅₋₂₀ alkenyl. In certain embodiments, R′ is substituted C₅₋₂₀ alkyl or C₅₋₂₀ alkenyl (e.g., substituted with a C₃₋₆ carbocycle such as 1-cyclopropylnonyl or substituted with OH or alkoxy). For example, R¹ is

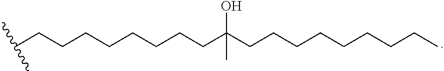 [SMILES: *CCCCCCCCC(C)(O)CCCCCCCCC]

In other embodiments, R¹ is —R″M′R′. In certain embodiments, M′ is —OC(O)-M″-C(O)O—. For example, R¹ is

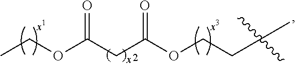

wherein x¹ is an integer between 1 and 13 (e.g., selected from 3, 4, 5, and 6), x² is an integer between 1 and 13 (e.g., selected from 1, 2, and 3), and x³ is an integer between 2 and 14 (e.g., selected from 4, 5, and 6). For example, x¹ is selected from 3, 4, 5, and 6, x² is selected from 1, 2, and 3, and x³ is selected from 4, 5, and 6.

In other embodiments, R¹ is different from —(CHR⁵R⁶)ₘ-M-CR²R³R⁷.

In some embodiments, R′ is selected from —R*YR″ and —YR″. In some embodiments, Y is C₃₋₈ cycloalkyl. In some embodiments, Y is C₆₋₁₀ aryl. In some embodiments, Y is a cyclopropyl group. In some embodiments, Y is a cyclohexyl group. In certain embodiments, R* is C₁ alkyl.

In some embodiments, R″ is selected from the group consisting of C₃₋₁₂ alkyl and C₃₋₁₂ alkenyl. In some embodiments, R″ is C₈ alkyl. In some embodiments, R″ adjacent to Y is C₁ alkyl. In some embodiments, R″ adjacent to Y is C₄₋₉ alkyl (e.g., C₄, C₅, C₆, C₇ or C₈ or C₉ alkyl).

In some embodiments, R″ is substituted C₃₋₁₂ (e.g., C₃₋₁₂ alkyl substituted with, e.g., an hydroxyl). For example, R″ is

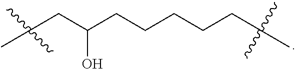 [SMILES: *CCCCCC(O)C*]

In some embodiments, R′ is selected from C₄ alkyl and C₄ alkenyl. In certain embodiments, R′ is selected from C₅ alkyl and C₅ alkenyl. In some embodiments, R′ is selected from C₆ alkyl and C₆ alkenyl. In some embodiments, R′ is selected from C₇ alkyl and C₇ alkenyl. In some embodiments, R′ is selected from C₉ alkyl and C₉ alkenyl.

In some embodiments, R′ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, CH alkenyl, C₁₇ alkyl, C₁₇ alkenyl, C₁₈ alkyl, and C₁₈ alkenyl, each of which is either linear or branched.

In some embodiments, R′ is linear. In some embodiments, R′ is branched.

In some embodiments, R′ is

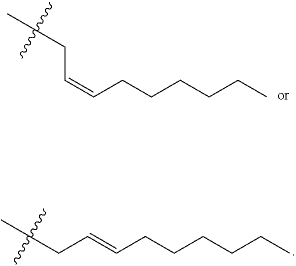

In some embodiments, R′ is

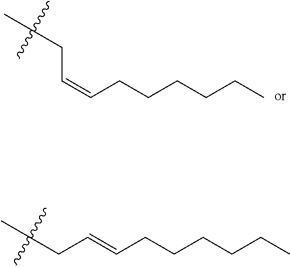

and M′ is —OC(O)—. In other embodiments, R′ is

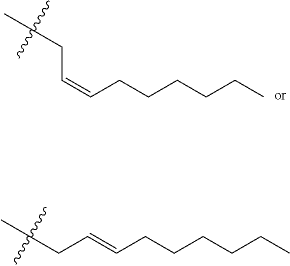

### and M′ is —C(O)O—.

In other embodiments, R′ is selected from Cᵢᵢ alkyl and C₁₁ alkenyl. In other embodiments, R′ is selected from Cu alkyl, C₁₂ alkenyl, C₁₃ alkyl, C₁₃ alkenyl, C₁₄ alkyl, C₁₄ alkenyl, C₁₅ alkyl, C₁₅ alkenyl, C₁₆ alkyl, C₁₆ alkenyl, C₁₇ alkyl, C₁₇ alkenyl, C₁₈ alkyl, and C₁₈ alkenyl. In certain embodiments, R′ is linear C₄₋₁₈ alkyl or C₄₋₁₈ alkenyl. In certain embodiments, R′ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl or heptadeca-9-yl). In certain embodiments, R′ is

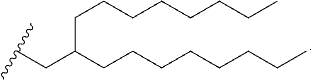 [SMILES: *CC(CCCCCCCC)CCCCCCCC]

In certain embodiments, R′ is unsubstituted C₁₋₁₈ alkyl. In certain embodiments, R′ is substituted C₁₋₁₈ alkyl (e.g., C₁₋₁₅ alkyl substituted with, e.g., an alkoxy such as methoxy, or a C₃₋₆ carbocycle such as 1-cyclopropylnonyl, or C(O)O-alkyl or OC(O)-alkyl such as C(O)OCH3 or OC(O)CH₃). For example, R′ is

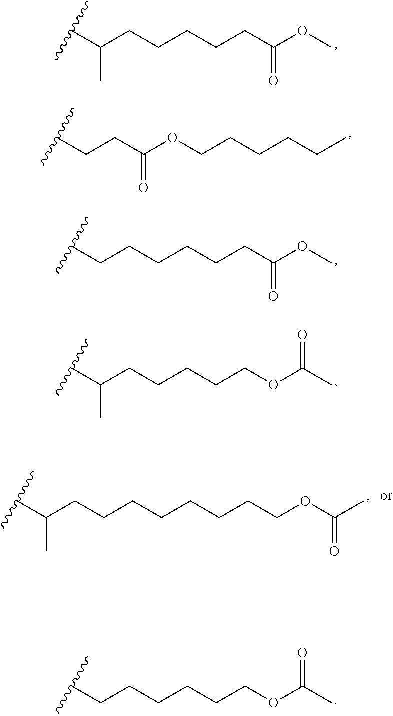 [SMILES: *C(C)CCCCCC(=O)OC ; *CCC(=O)OCCCCCC ; *CCCCCCC(=O)OC ; *C(C)CCCCCOC(C)=O ; *C(C)CCCCCCCCOC(C)=O ; *CCCCCCOC(C)=O]

In certain embodiments, R′ is branched C₁₋₁₈ alkyl. For example, R′ is

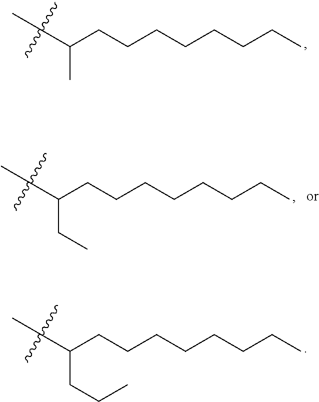 [SMILES: *C(C)CCCCCCCC ; *C(CC)CCCCCCCC ; *C(CCC)CCCCCCCC]

In some embodiments, R″ is selected from the group consisting of C₃₋₁₅ alkyl and C₃₋₁₅ alkenyl. In some embodiments, R″ is C₃ alkyl, C₄ alkyl, C₅ alkyl, C₆ alkyl, C₇ alkyl, or C₈ alkyl.

In some embodiments, R″ is C₉ alkyl, C₁₀ alkyl, C₁₁ alkyl, C₁₂ alkyl, C₁₃ alkyl, C₁₄ alkyl, or Cis alkyl.

In some embodiments, M′ is —C(O)O—. In some embodiments, M′ is —OC(O)—. In some embodiments, M′ is —OC(O)-M″-C(O)O—.

In some embodiments, M′ is —C(O)O—, —OC(O)—, or —OC(O)-M″-C(O)O—. In some embodiments wherein M′ is —OC(O)-M″-C(O)O—, M″ is C₁₋₄ alkyl or C₂₋₄ alkenyl.

In other embodiments, M′ is an aryl group or heteroaryl group. For example, M′ may be selected from the group consisting of phenyl, oxazole, and thiazole.

In some embodiments, M is —C(O)O—. In some embodiments, M is —OC(O)—. In some embodiments, M is —C(O)N(R′)—. In some embodiments, M is —P(O)(OR′)O—. In some embodiments, M is —OC(O)-M″-C(O)O—.

In some embodiments, M is —C(O). In some embodiments, M is —OC(O)— and M′ is —C(O)O—. In some embodiments, M is —C(O)O— and M′ is —OC(O)—. In some embodiments, M and M′ are each —OC(O)—. In some embodiments, M and M′ are each —C(O)O—.

In other embodiments, M is an aryl group or heteroaryl group. For example, M may be selected from the group consisting of phenyl, oxazole, and thiazole.

In some embodiments, M is the same as M′. In other embodiments, M is different from M.

In some embodiments, M″ is a bond. In some embodiments, M″ is C₁₋₁₃ alkyl or C₂₋₁₃ alkenyl. In some embodiments, M″ is C₁₋₆ alkyl or C₂₋₆ alkenyl. In certain embodiments, M″ is linear alkyl or alkenyl. In certain embodiments. M″ is branched, e.g., —CH(CH₃)CH₂—.

In some embodiments, each R⁵ is H. In some embodiments, each R⁶ is H. In certain such embodiments, each R⁵ and each R⁶ is H.

In some embodiments, R⁷ is H. In other embodiments, R⁷ is C₁₋₃ alkyl (e.g., methyl, ethyl, propyl, or i-propyl).

In some embodiments, R² and R³ are independently C₅₋₁₄ alkyl or C₅₋₁₄ alkenyl.

In some embodiments, R² and R³ are the same. In some embodiments, R² and R³ are C₈ alkyl. In certain embodiments, R² and R³ are C₂ alkyl. In other embodiments, R² and R³ are C₃ alkyl. In some embodiments, R² and R³ are C₄ alkyl. In certain embodiments, R² and R³ are C₅ alkyl. In other embodiments, R² and R³ are C₆ alkyl. In some embodiments, R² and R³ are C₇ alkyl.

In other embodiments, R² and R³ are different. In certain embodiments, R² is C₈ alkyl. In some embodiments, R³ is C₁₋₇ (e.g., C₁, C₂, C₃, C₄, C₅, C₆, or C₇ alkyl) or C₉ alkyl.

In some embodiments, R³ is C₁ alkyl. In some embodiments, R³ is C₂ alkyl. In some embodiments, R³ is C₃ alkyl. In some embodiments, R³ is C₄ alkyl. In some embodiments, R³ is C₅ alkyl. In some embodiments, R³ is C₆ alkyl. In some embodiments, R³ is C₇ alkyl. In some embodiments, R³ is C₉ alkyl.

In some embodiments, R⁷ and R³ are H.

In certain embodiments, R² is H.

In some embodiments, m is 5, 6, 7, 8, or 9. In some embodiments, m is 5, 7, or 9. For example, in some embodiments, m is 5. For example, in some embodiments, m is 7. For example, in some embodiments, m is 9.

In some embodiments, R⁴ is selected from —(CH₂)ₙQ and —(CH₂)ₙCHQR,

In some embodiments, Q is selected from the group consisting of —OR, —OH, —O(CH₂)ₙN(R)₂, —OC(O)R, —CX3. —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), —C(R)N(R)₂C(O)OR, —N(R)S(O)₂R⁸, a carbocycle, and a heterocycle.

In certain embodiments, Q is —N(R)R⁸, —N(R)S(O)₂R⁸, —O(CH₂)ₙOR, —N(R)C(═NR⁹)N(R)₂. —N(R)C(═CHR⁹)N(R)₂, —OC(O)N(R)₂, or —N(R)C(O)OR.

In certain embodiments, Q is —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, or —N(OR)C(═CHR⁹)N(R)₂.

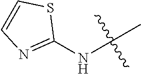 [SMILES: *Nc1nccs1]

In certain embodiments, Q is thiourea or an isostere thereof, e.g., or —NHC(═NR⁹)N(R)₂.

In certain embodiments, Q is —C(═NR⁹)N(R)₂. For example, when Q is —C(═NR⁹)N(R)₂,
n is 4 or 5. For example, R⁹ is —S(O)₂N(R)₂.

In certain embodiments, Q is —C(═NR⁹)R or —C(O)N(R)OR, e.g., —CH(═N—OCH3), —C(O)NH—OH, —C(O)NH—OCH₃, —C(O)N(CH₃)—OH, or —C(O)N(CH₃)—OCH₃.

In certain embodiments, Q is —OH.

In certain embodiments, Q is a substituted or unsubstituted 5- to 10-membered heteroaryl, e.g., Q is a triazole, an imidazole, a pyrimidine, a purine, 2-amino-1,9-dihydro-6H-purin-6-one-9-yl (or guanin-9-yl), adenin-9-yl, cytosin-1-yl, or uracil-1-yl, each of which is optionally substituted with one or more substituents selected from alkyl, OH, alkoxy, -alkyl-OH, -alkyl-O-alkyl, and the substituent can be further substituted. In certain embodiments, Q is a substituted 5- to 14-membered heterocycloalkyl, e.g., substituted with one or more substituents selected from oxo (═O), OH, amino, mono- or di-alkylamino, and C₁₋₃ alkyl. For example, Q is 4-methylpiperazinyl, 4-(4-methoxybenzyl)piperazinyl, isoindolin-2-yl-1,3-dione, pyrrolidin-1-yl-2,5-dione, or imidazolidin-3-yl-2,4-dione.

In certain embodiments, Q is —NHR⁸, in which R⁸ is a C₃₋₆ cycloalkyl optionally substituted with one or more substituents selected from oxo (═O), amino (NH₂), mono- or di-alkylamino, C₁₋₃ alkyl and halo. For example, R⁸ is cyclobutenyl, e.g., 3-(dimethylamino)-cyclobut-3-ene-4-yl-1,2-dione. In further embodiments, R⁸ is a C₃₋₆ cycloalkyl optionally substituted with one or more substituents selected from oxo (═O), thio (═S), amino (NH₂), mono- or di-alkylamino, C₁₋₃ alkyl, heterocycloalkyl, and halo, wherein the mono- or di-alkylamino, C₁₋₃ alkyl, and heterocycloalkyl are further substituted. For example R⁸ is cyclobutenyl substituted with one or more of oxo, amino, and alkylamino, wherein the alkylamino is further substituted, e.g., with one or more of C₁₋₃ alkoxy, amino, mono- or di-alkylamino, and halo. For example, R⁸ is 3-(((dimethylamino)ethyl)amino)cyclobut-3-enyl-1,2-dione. For example R⁸ is cyclobutenyl substituted with one or more of oxo, and alkylamino. For example, R⁸ is 3-(ethylamino)cyclobut-3-ene-1,2-dione. For example R⁸ is cyclobutenyl substituted with one or more of oxo, thio, and alkylamino. For example R⁸ is 3-(ethylamino)-4-thioxocyclobut-2-en-1-one or 2-(ethylamino)-4-thioxocyclobut-2-en-1-one. For example R⁸ is cyclobutenyl substituted with one or more of thio, and alkylamino. For example R⁸ is 3-(ethylamino)cyclobut-3-ene-1,2-dithione. For example R⁸ is cyclobutenyl substituted with one or more of oxo and dialkylamino. For example R⁸ is 3-(diethylamino)cyclobut-3-ene-1,2-dione. For example, R⁸ is cyclobutenyl substituted with one or more of oxo, thio, and dialkylamino. For example, R⁸ is 2-(diethylamino)-4-thioxocyclobut-2-en-1-one or 3-(diethylamino)-4-thioxocyclobut-2-en-1-one. For example, R⁸ is cyclobutenyl substituted with one or more of thio, and dialkylamino. For example, R⁸ is 3-(diethylamino)cyclobut-3-ene-1,2-dithione. For example, R⁸ is cyclobutenyl substituted with one or more of oxo and alkylamino or dialkylamino, wherein alkylamino or dialkylamino is further substituted, e.g. with one or more alkoxy. For example, R⁸ is 3-(bis(2-methoxyethyl)amino)cyclobut-3-ene-1,2-dione. For example, R⁸ is cyclobutenyl substituted with one or more of oxo, and heterocycloalkyl. For example, R⁸ is cyclobutenyl substituted with one or more of oxo, and piperidinyl, piperazinyl, or morpholinyl. For example, R⁸ is cyclobutenyl substituted with one or more of oxo, and heterocycloalkyl, wherein heterocycloalkyl is further substituted, e.g., with one or more C₁₋₃ alkyl. For example, R⁸ is cyclobutenyl substituted with one or more of oxo, and heterocycloalkyl, wherein heterocycloalkyl (e.g., piperidinyl, piperazinyl, or morpholinyl) is further substituted with methyl.

In certain embodiments, Q is —NHR⁸, in which R⁸ is a heteroaryl optionally substituted with one or more substituents selected from amino (NH₂), mono- or di-alkylamino, C₁₋₃ alkyl and halo. For example, R⁸ is thiazole or imidazole.

In certain embodiments, Q is —NHC(═NR⁹)N(R)₂ in which R⁹ is CN, C₁₋₆ alkyl, NO₂, —S(O)₂N(R)₂, —OR, —S(O)₂R, or H. For example, Q is —NHC(═NR⁹)N(CH₃)₂, —NHC(═NR⁹)NHCH₃, —NHC(═NR⁹)NH₂. In some embodiments, Q is —NHC(═NR⁹)N(R)₂ in which R⁹ is CN and R is C₁₋₃ alkyl substituted with mono- or di-alkylamino, e.g., R is ((dimethylamino)ethyl)amino. In some embodiments, Q is —NHC(═NR⁹)N(R)₂ in which R⁹ is C₁₋₆ alkyl, NO₂, —S(O)₂N(R)₂, —OR, —S(O)₂R, or H and R is C₁₋₃ alkyl substituted with mono- or di-alkylamino, e.g., R is ((dimethylamino)ethyl)amino.

In certain embodiments, Q is —NHC(═CHR⁹)N(R)₂, in which R⁹ is NO₂, CN, C₁₋₆ alkyl, —S(O)₂N(R)₂, —OR, —S(O)₂R, or H. For example, Q is —NHC(═CHR⁹)N(CH₃)₂, —NHC(═CHR⁹)NHCH₃, or —NHC(═CHR⁹)NH₂.

In certain embodiments, Q is —OC(O)N(R)₂, —N(R)C(O)OR, —N(OR)C(O)OR, such as —OC(O)NHCH₃, —N(OH)C(O)OCH₃, —N(OH)C(O)CH₃, —N(OCH₃)C(O)OCH₃. —N(OCH₃)C(O)CH₃, —N(OH)S(O)₂CH₃, or —NHC(O)OCH₃.

In certain embodiments, Q is —N(R)C(O)R, in which R is alkyl optionally substituted with C₁₋₃ alkoxyl or S(O)_{z}C₁₋₃ alkyl, in which z is 0, 1, or 2.

In certain embodiments, Q is an unsubstituted or substituted C₆₋₁₀ aryl (such as phenyl) or C₃₋₆ cycloalkyl.

In some embodiments, n is 1. In other embodiments, n is 2. In further embodiments, n is 3. In certain other embodiments, n is 4. For example, R⁴ may be —(CH₂)₂OH. For example, R⁴ may be —(CH₂)₃OH. For example, R⁴ may be —(CH₂)₄OH. For example, R⁴ may be benzyl. For example, R⁴ may be 4-methoxybenzyl.

In some embodiments, R⁴ is a C₃₋₆ carbocycle. In some embodiments, R⁴ is a C₃₋₆ cycloalkyl. For example, R⁴ may be cyclohexyl optionally substituted with e.g., OH, halo, C₁₋₆ alkyl, etc. For example, R⁴ may be 2-hydroxycyclohexyl.

In some embodiments, R is H.

In some embodiments, R is C₁₋₃ alkyl substituted with mono- or di-alkylamino, e.g., R is ((dimethylamino)ethyl)amino.

In some embodiments, R is C₁₋₆ alkyl substituted with one or more substituents selected from the group consisting of C₁₋₃ alkoxyl, amino, and C₁-C₃ dialkylamino.

In some embodiments, R is unsubstituted C₁₋₃ alkyl or unsubstituted C₂₋₃ alkenyl. For example, R⁴ may be —CH₂CH(OH)CH₃, —CH(CH₃)CH₂OH, or —CH₂CH(OH)CH₂CH₃.

In some embodiments, R is substituted C₁₋₃ alkyl, e.g., CH₂OH. For example, R⁴ may be —CH₂CH(OH)CH₂OH, —(CH₂)₃NHC(O)CH₂OH, —(CH₂)₃NHC(O)CH₂OBn, —(CH₂)₂O(CH₂)₂OH, —(CH₂)₃NHCH₂OCH₃, —(CH₂)₃NHCH₂OCH₂CH₃, CH₂SCH₃, CH₂S(O)CH₃, CH₂S(O)₂CH₃, or —CH(CH₂OH)₂.

In some embodiments, R⁴ is selected from any of the following groups:

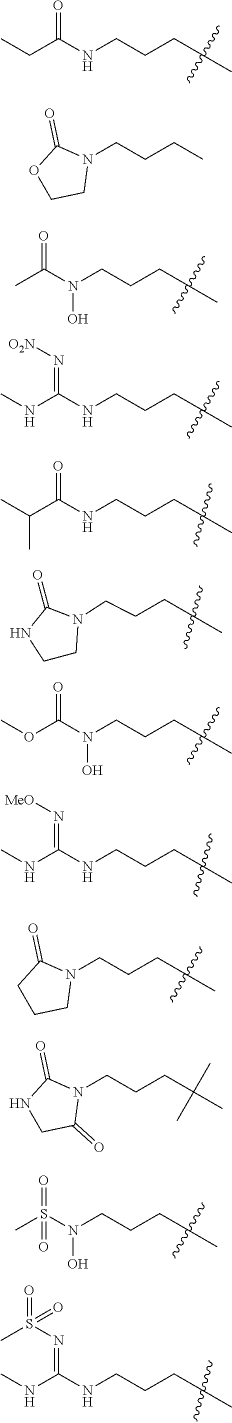

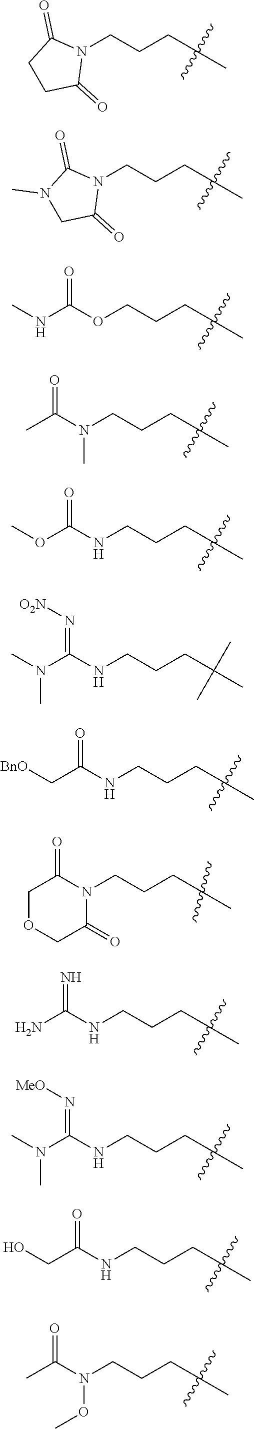

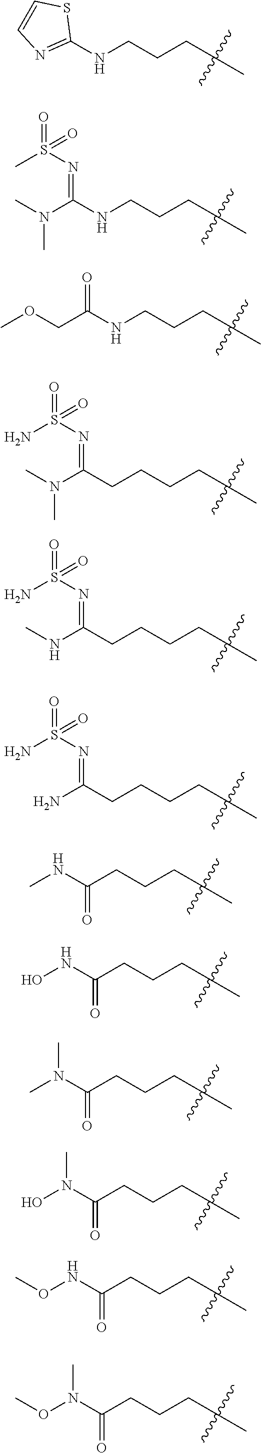

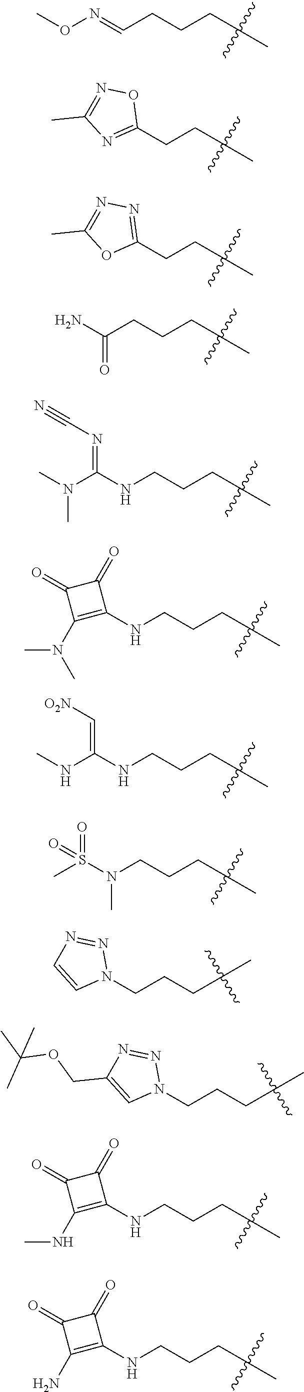

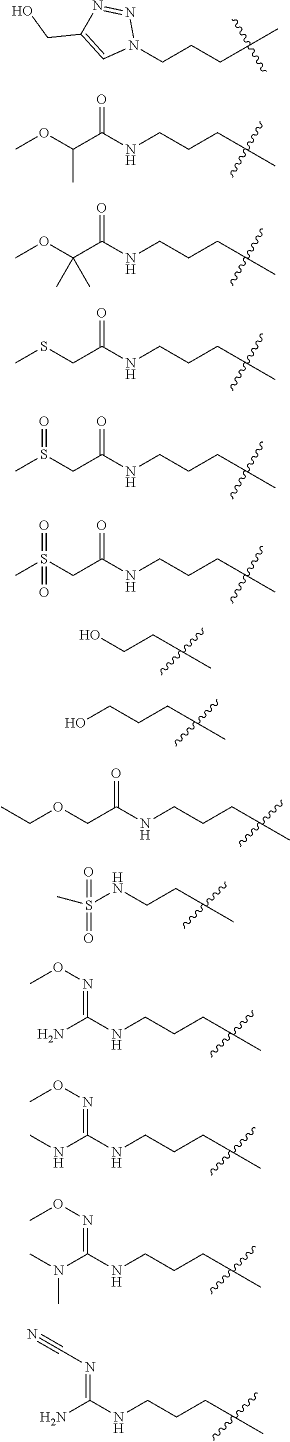

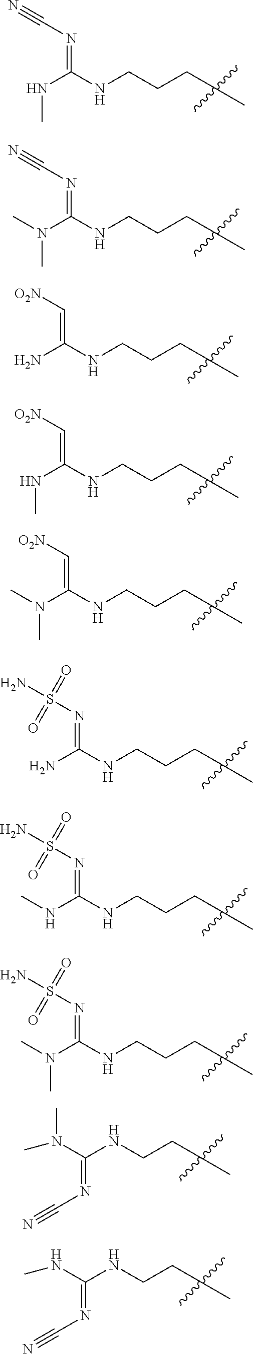

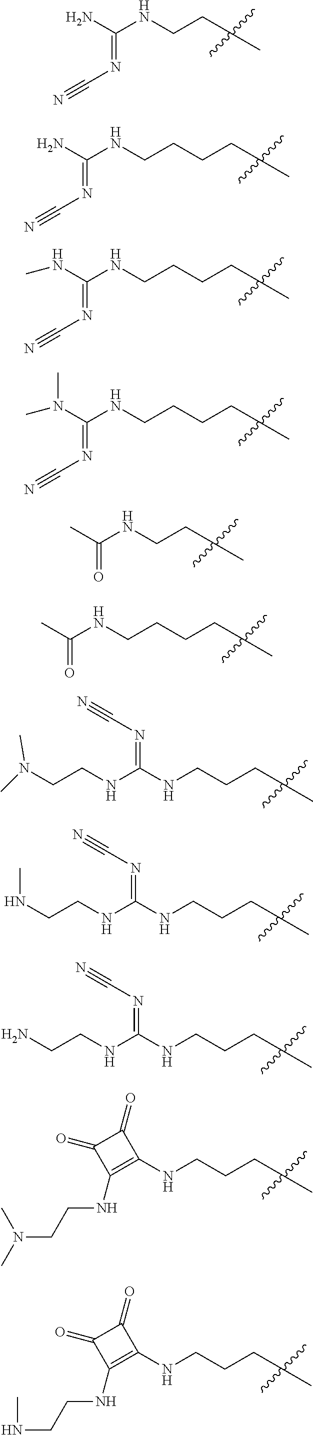

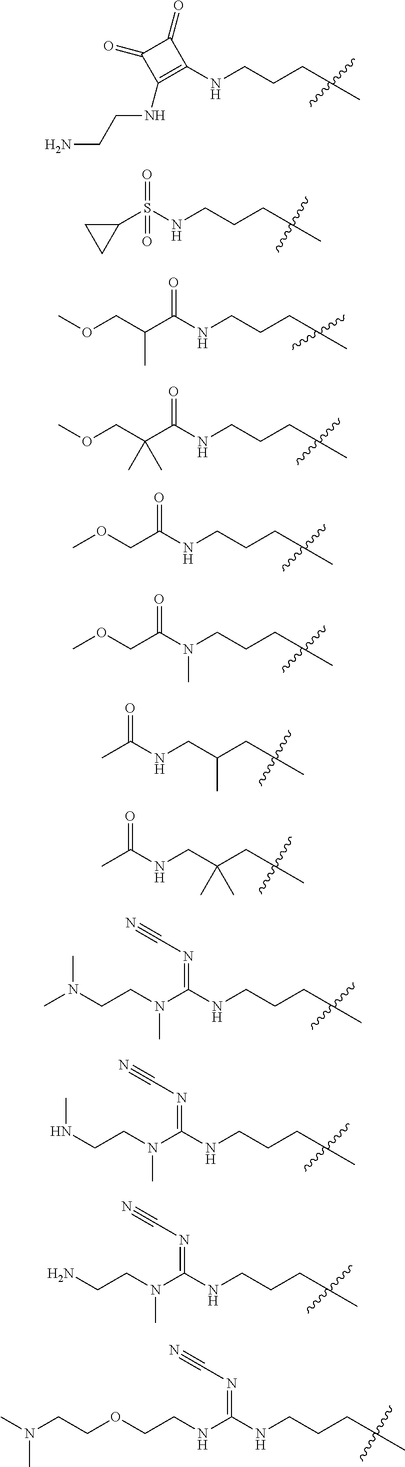

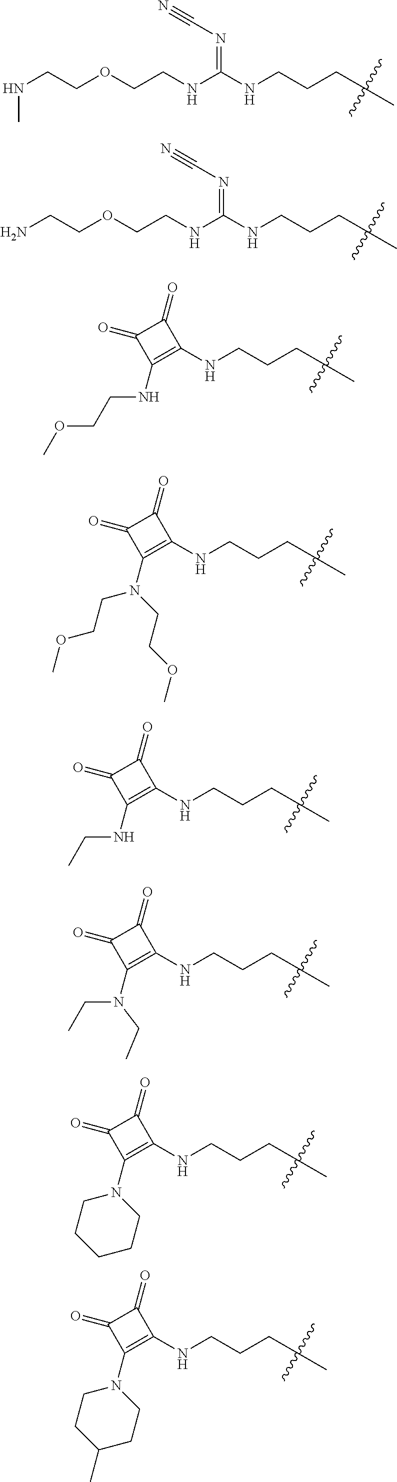

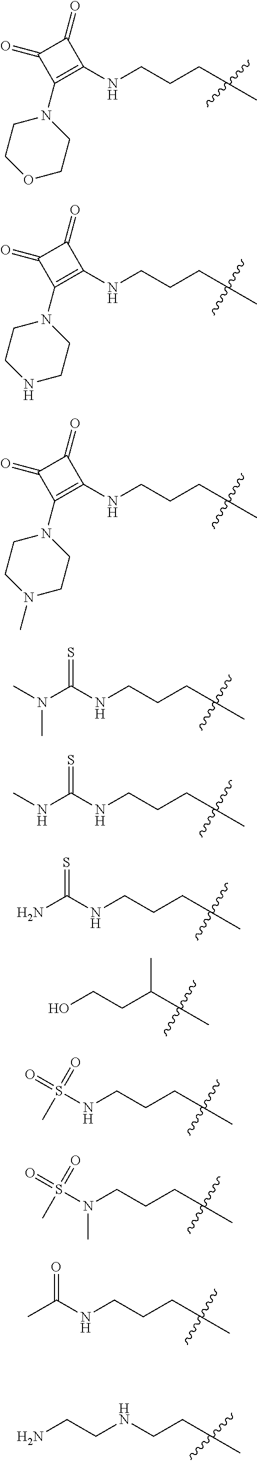

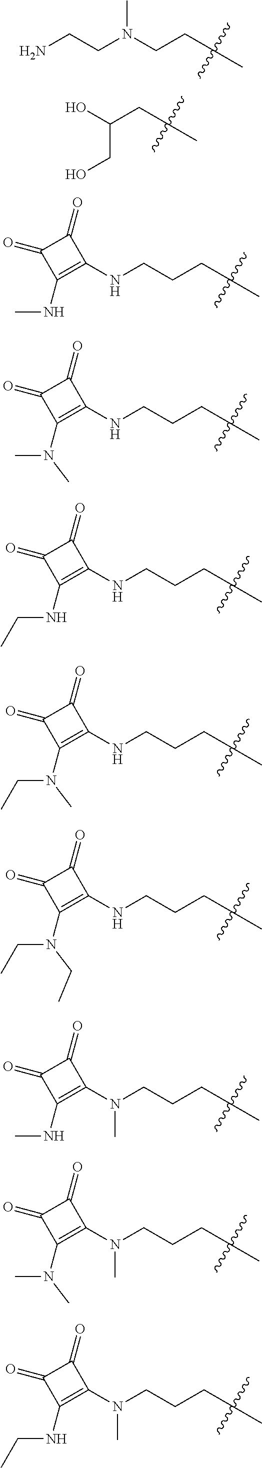

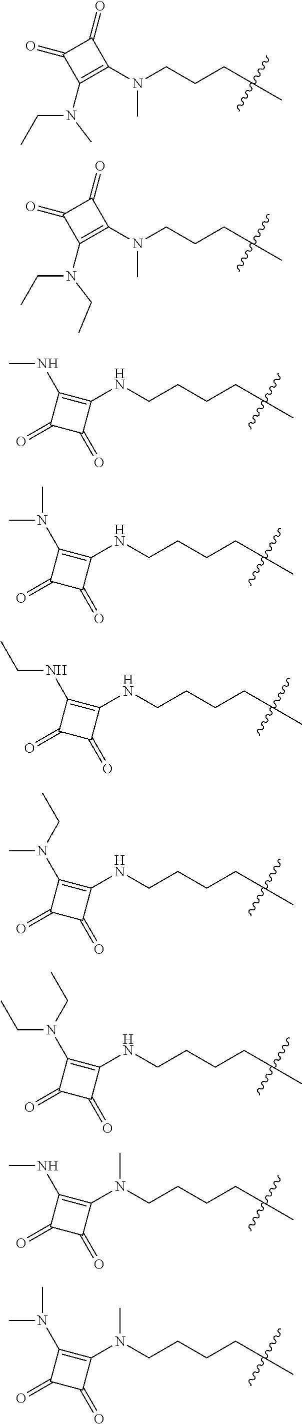

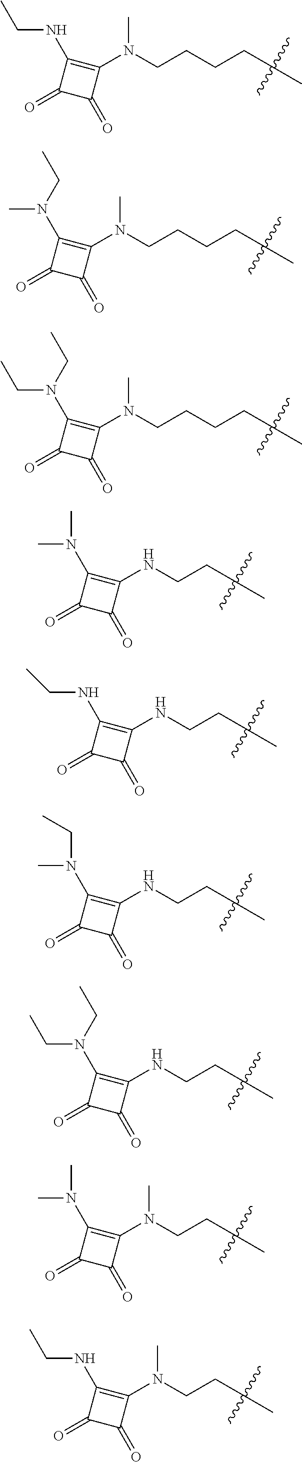

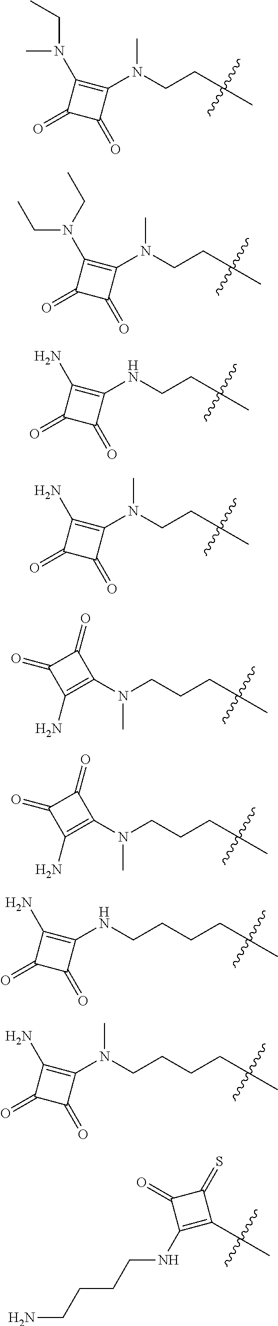

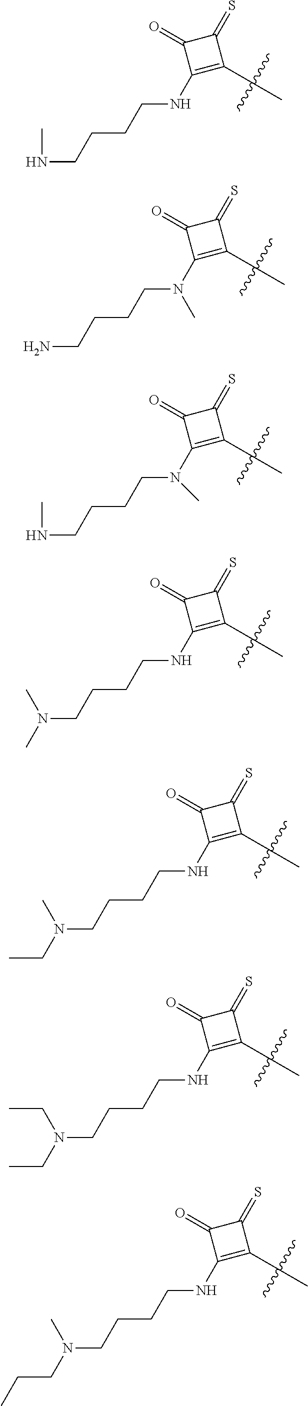

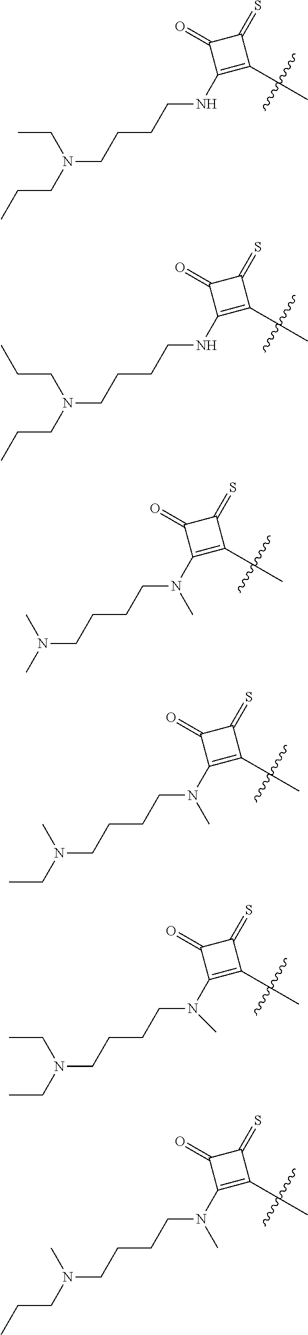 [SMILES: *c1c(NCCCCN(CC)CCC)c(=O)c1=S ; *c1c(NCCCCN(CCC)CCC)c(=O)c1=S ; *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S ; *c1c(N(C)CCCCN(C)CCC)c(=O)c1=S]

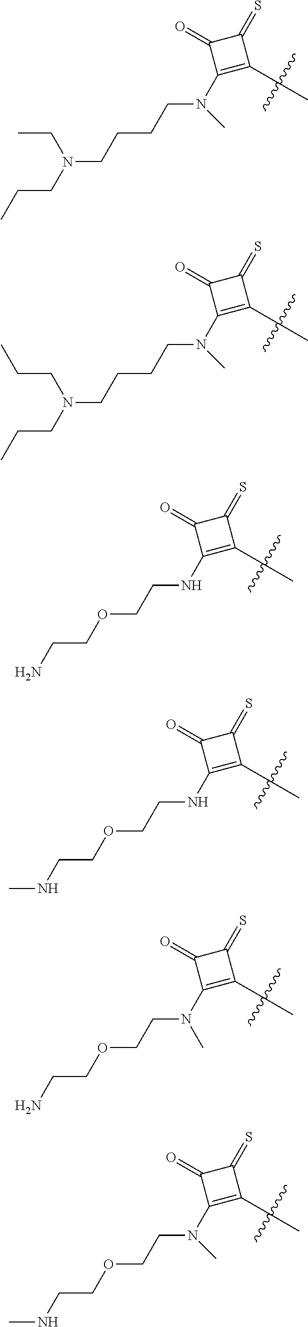 [SMILES: *c1c(N(C)CCCCN(CC)CCC)c(=O)c1=S ; *c1c(N(C)CCCCN(CCC)CCC)c(=O)c1=S ; *c1c(NCCOCCN)c(=O)c1=S ; *c1c(NCCOCCNC)c(=O)c1=S ; *c1c(N(C)CCOCCN)c(=O)c1=S ; *c1c(N(C)CCOCCNC)c(=O)c1=S]

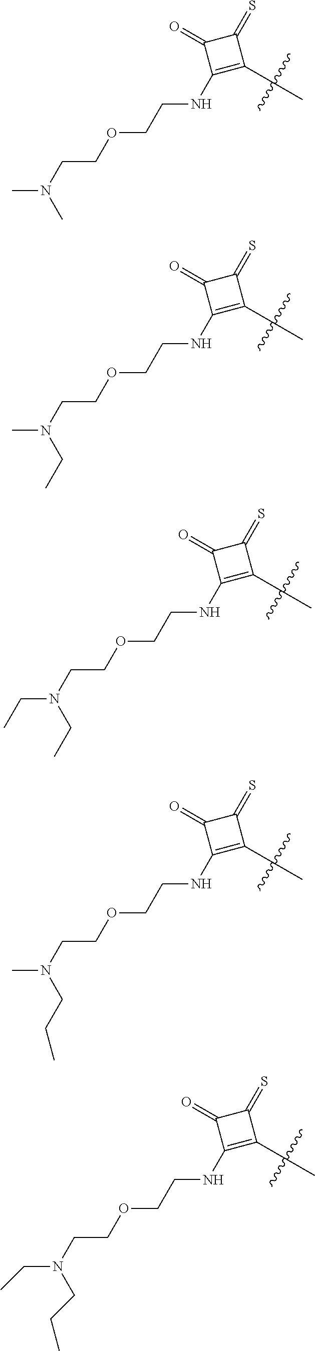 [SMILES: *c1c(NCCOCCN(C)C)c(=O)c1=S ; *c1c(NCCOCCN(C)CC)c(=O)c1=S ; *c1c(NCCOCCN(CC)CC)c(=O)c1=S ; *c1c(NCCOCCN(C)CCC)c(=O)c1=S ; *c1c(NCCOCCN(CC)CCC)c(=O)c1=S]

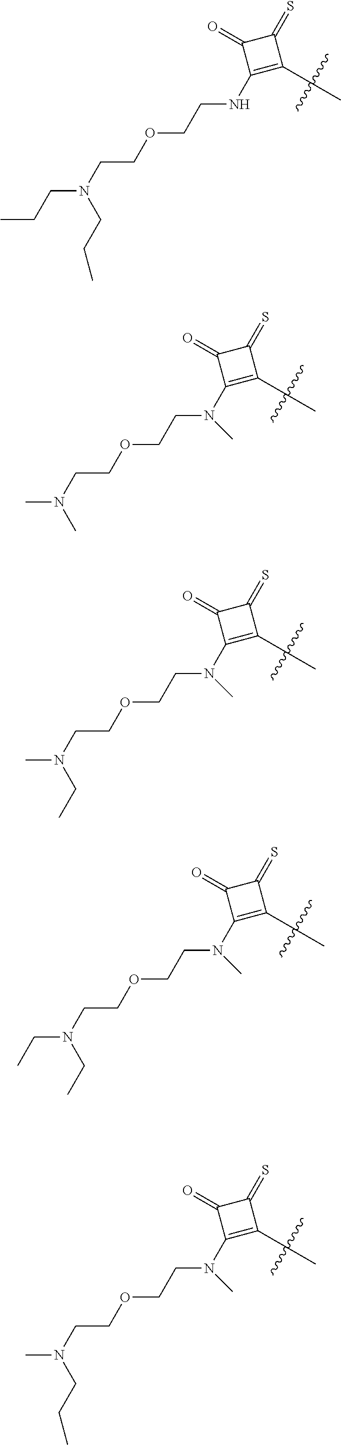 [SMILES: *c1c(NCCOCCN(CCC)CCC)c(=O)c1=S ; *c1c(N(C)CCOCCN(C)C)c(=O)c1=S ; *c1c(N(C)CCOCCN(C)CC)c(=O)c1=S ; *c1c(N(C)CCOCCN(CC)CC)c(=O)c1=S ; *c1c(N(C)CCOCCN(C)CCC)c(=O)c1=S]

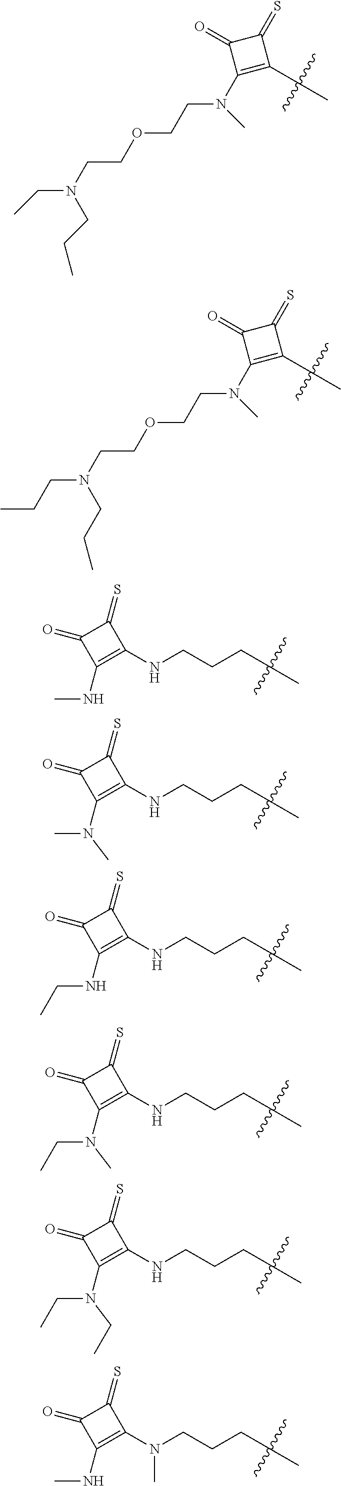

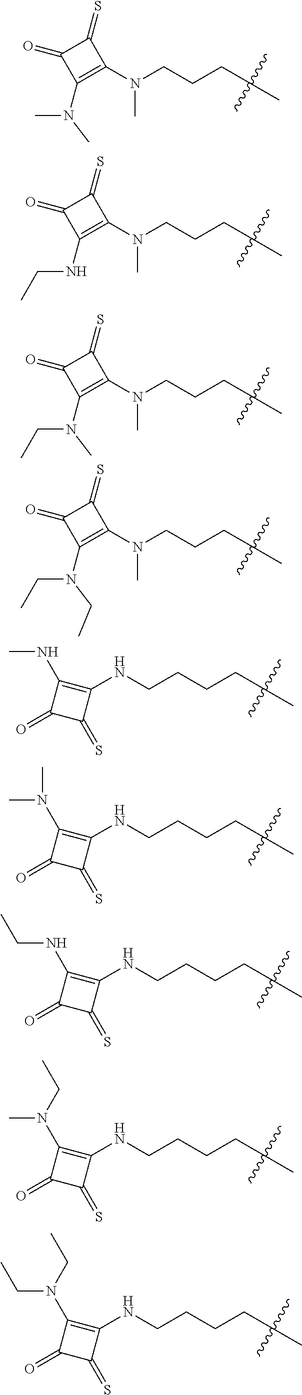

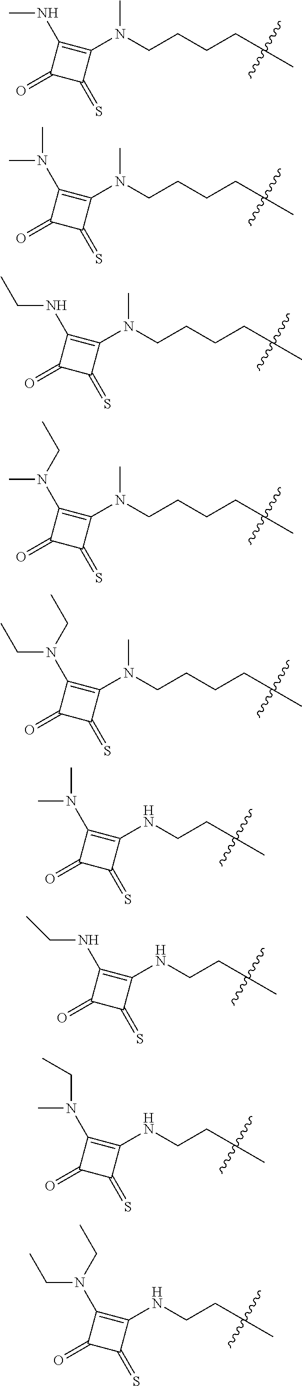

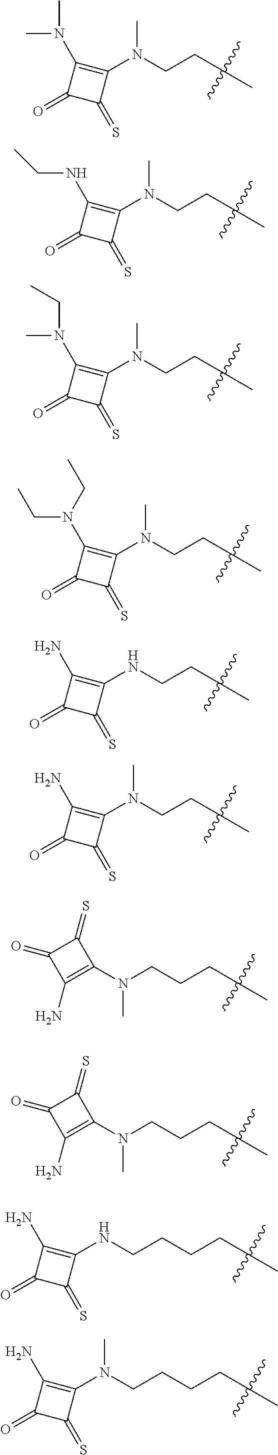

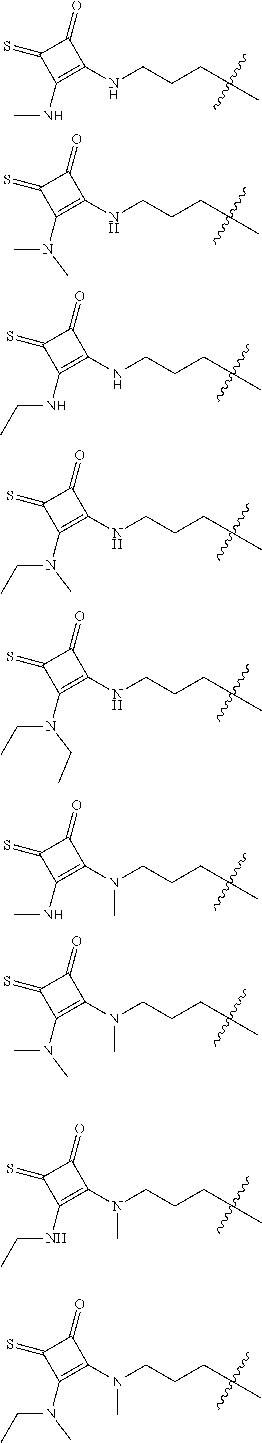

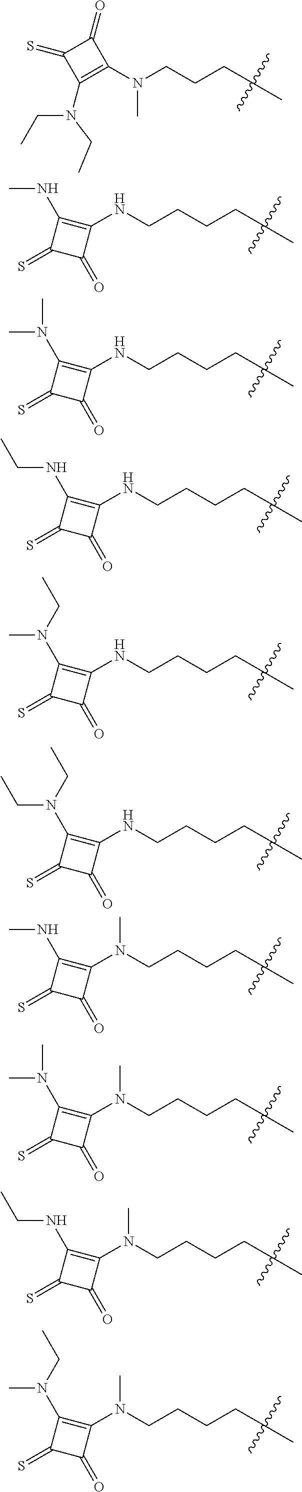

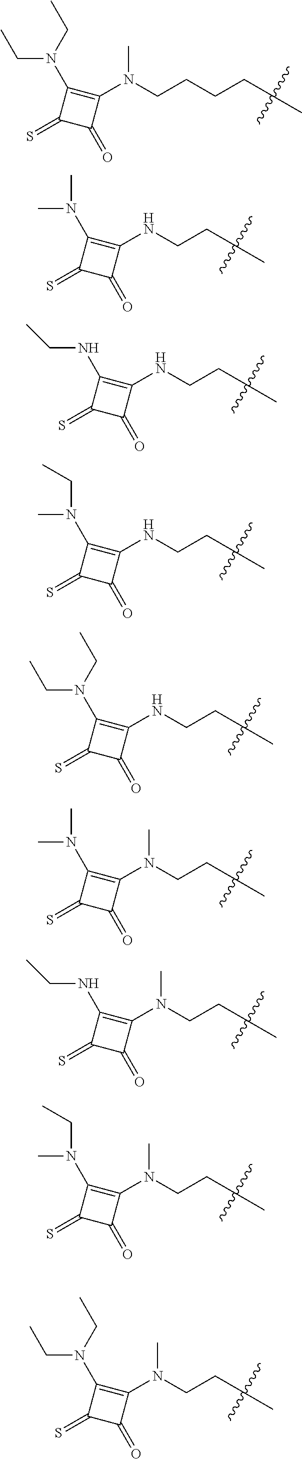

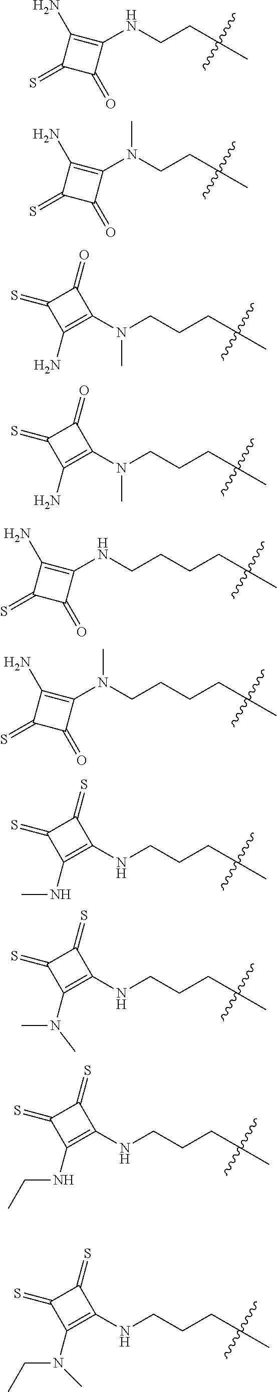

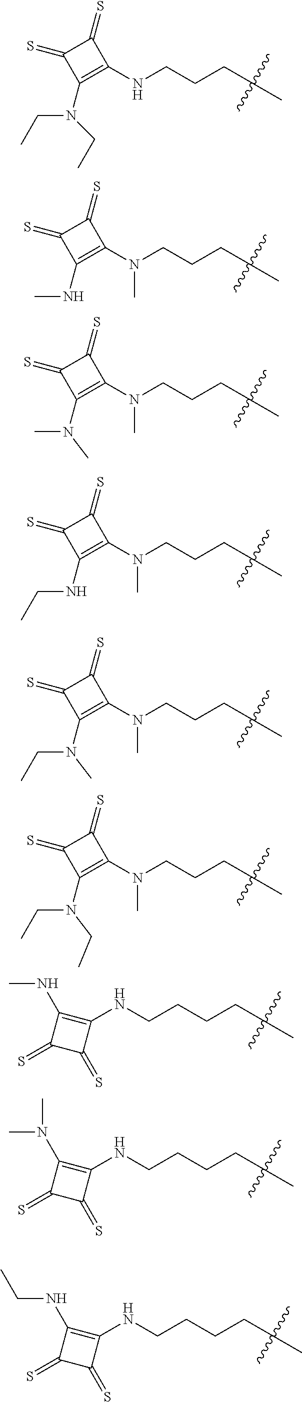

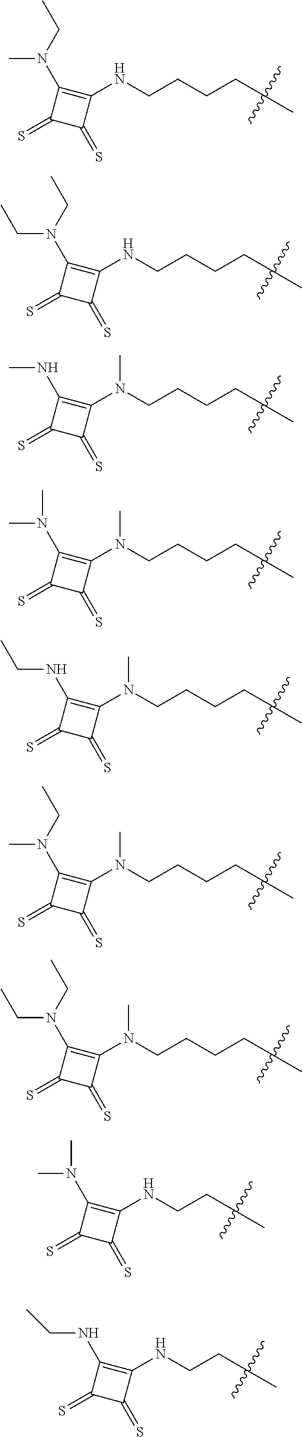

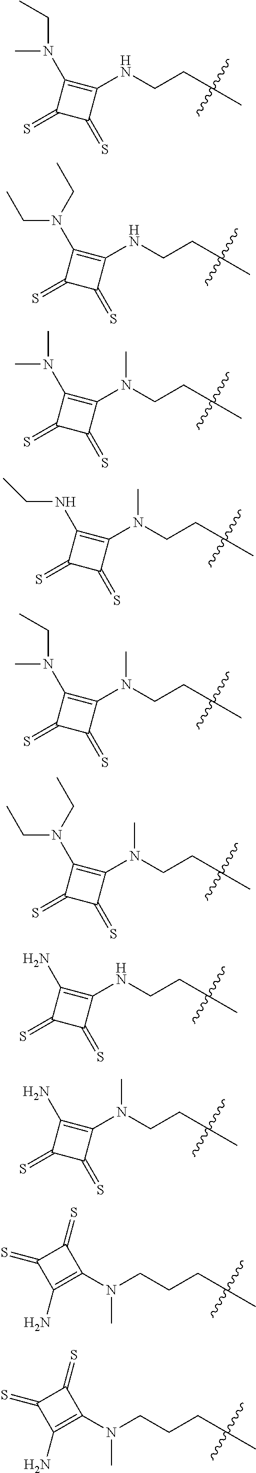

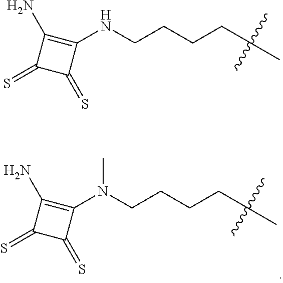 [SMILES: *CCCCNc1c(N)c(=S)c1=S ; *CCCCN(C)c1c(N)c(=S)c1=S]

In some embodiments,

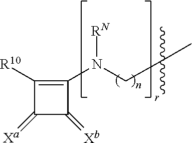

is selected from any of the following groups:

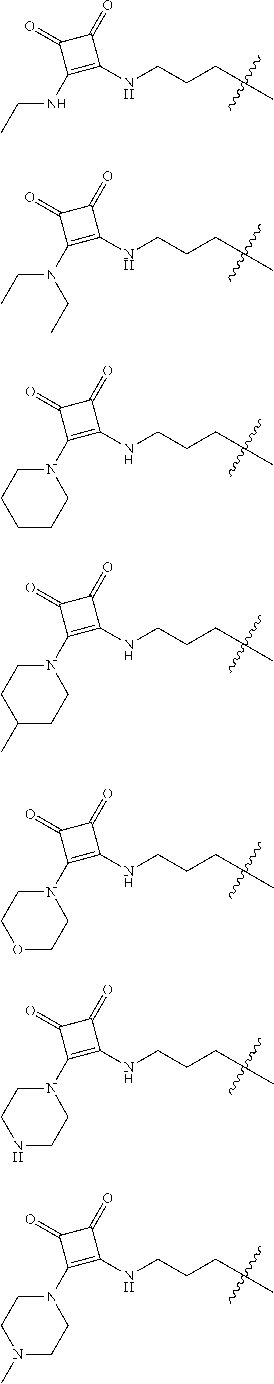

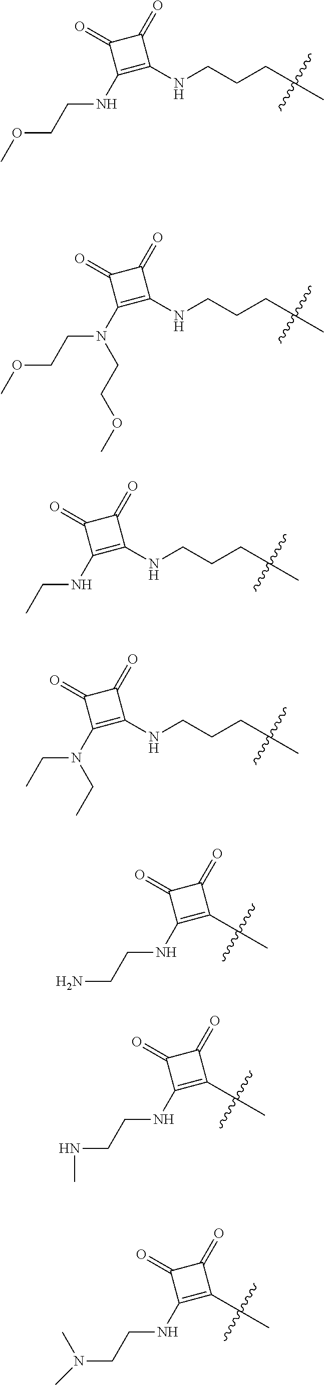

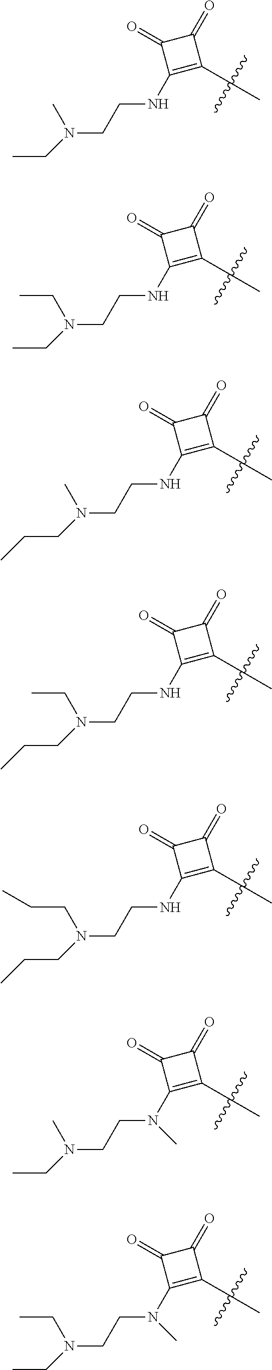

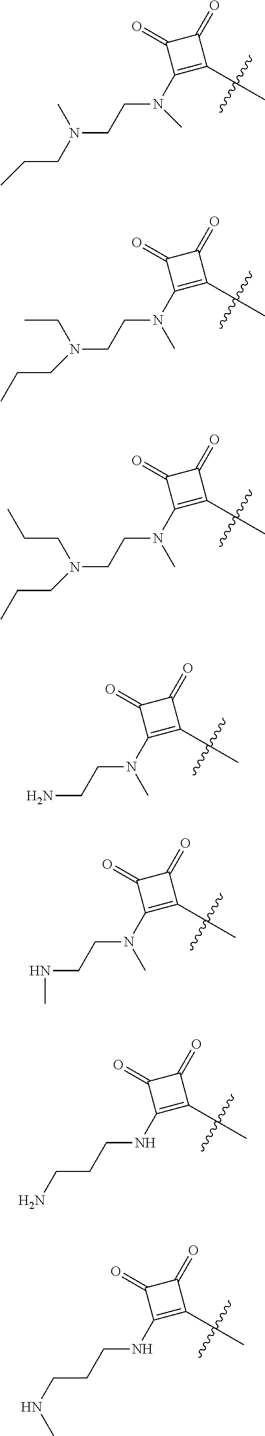

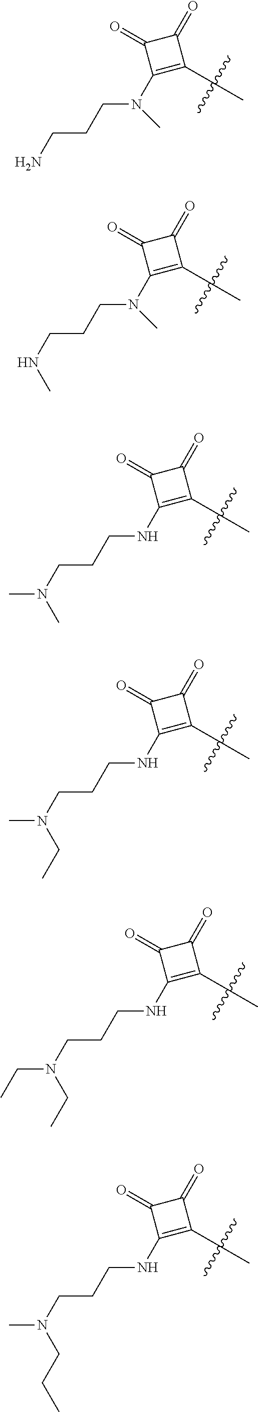 [SMILES: *c1c(N(C)CCCN)c(=O)c1=O ; *c1c(N(C)CCCNC)c(=O)c1=O ; *c1c(NCCCN(C)C)c(=O)c1=O ; *c1c(NCCCN(C)CC)c(=O)c1=O ; *c1c(NCCCN(CC)CC)c(=O)c1=O ; *c1c(NCCCN(C)CCC)c(=O)c1=O]

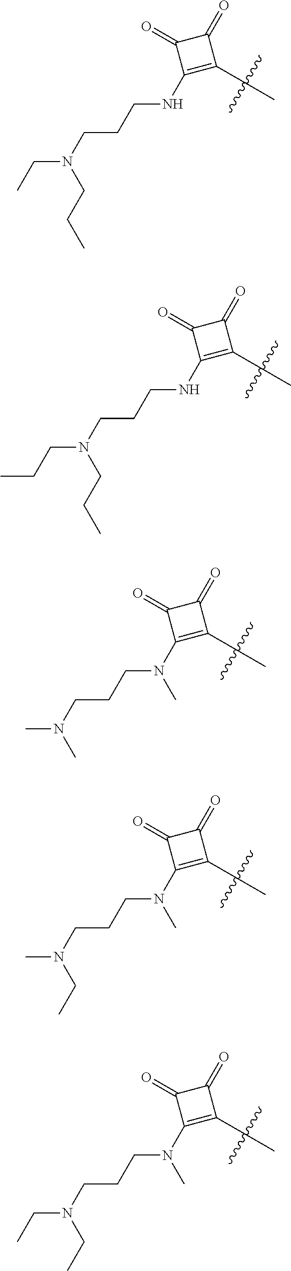 [SMILES: *c1c(NCCCN(CC)CCC)c(=O)c1=O ; *c1c(NCCCN(CCC)CCC)c(=O)c1=O ; *c1c(N(C)CCCN(C)C)c(=O)c1=O ; *c1c(N(C)CCCN(C)CC)c(=O)c1=O ; *c1c(N(C)CCCN(CC)CC)c(=O)c1=O]

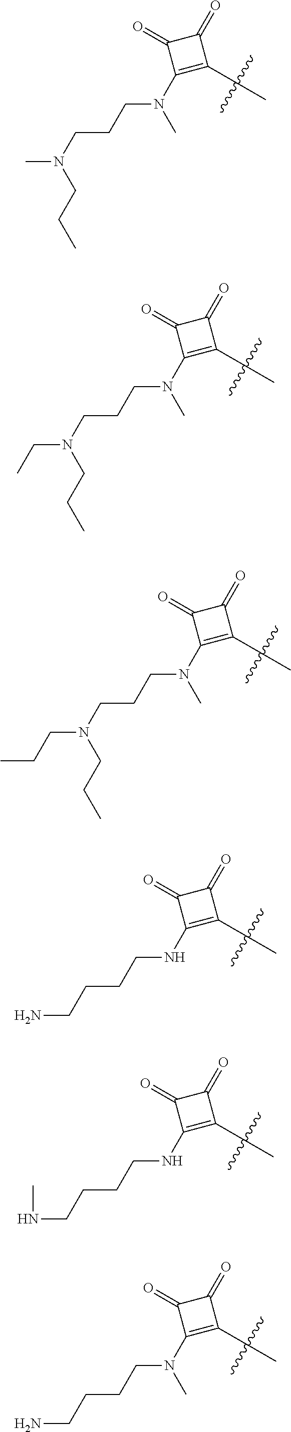 [SMILES: *c1c(N(C)CCCN(C)CCC)c(=O)c1=O ; *c1c(N(C)CCCN(CC)CCC)c(=O)c1=O ; *c1c(N(C)CCCN(CCC)CCC)c(=O)c1=O ; *c1c(NCCCCN)c(=O)c1=O ; *c1c(NCCCCNC)c(=O)c1=O ; *c1c(N(C)CCCCN)c(=O)c1=O]

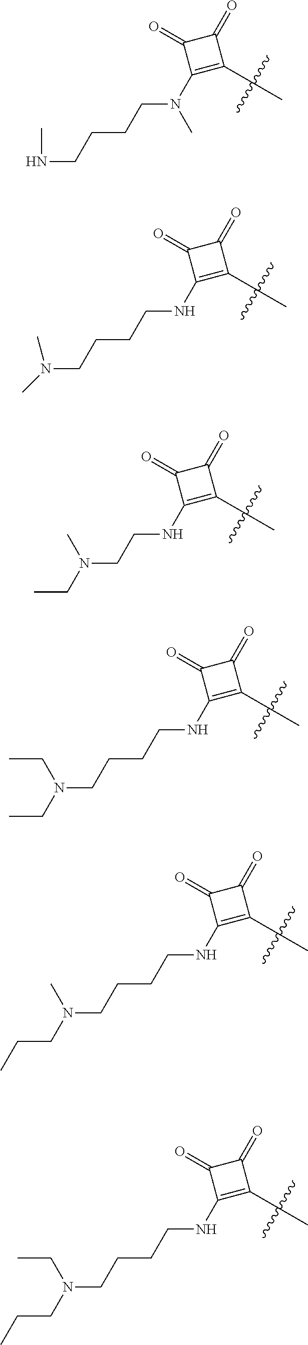 [SMILES: *c1c(N(C)CCCCNC)c(=O)c1=O ; *c1c(NCCCCN(C)C)c(=O)c1=O ; *c1c(NCCN(C)CC)c(=O)c1=O ; *c1c(NCCCCN(CC)CC)c(=O)c1=O ; *c1c(NCCCCN(C)CCC)c(=O)c1=O ; *c1c(NCCCCN(CC)CCC)c(=O)c1=O]

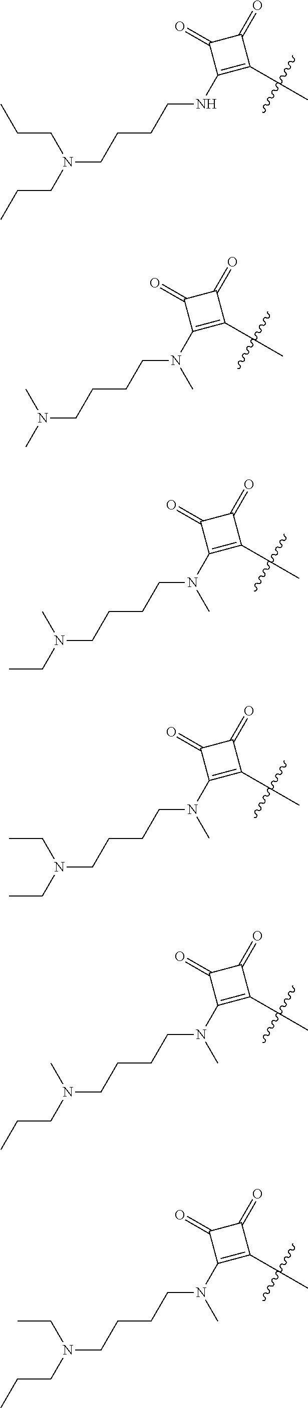 [SMILES: *c1c(NCCCCN(CCC)CCC)c(=O)c1=O ; *c1c(N(C)CCCCN(C)C)c(=O)c1=O ; *c1c(N(C)CCCCN(C)CC)c(=O)c1=O ; *c1c(N(C)CCCCN(CC)CC)c(=O)c1=O ; *c1c(N(C)CCCCN(C)CCC)c(=O)c1=O ; *c1c(N(C)CCCCN(CC)CCC)c(=O)c1=O]

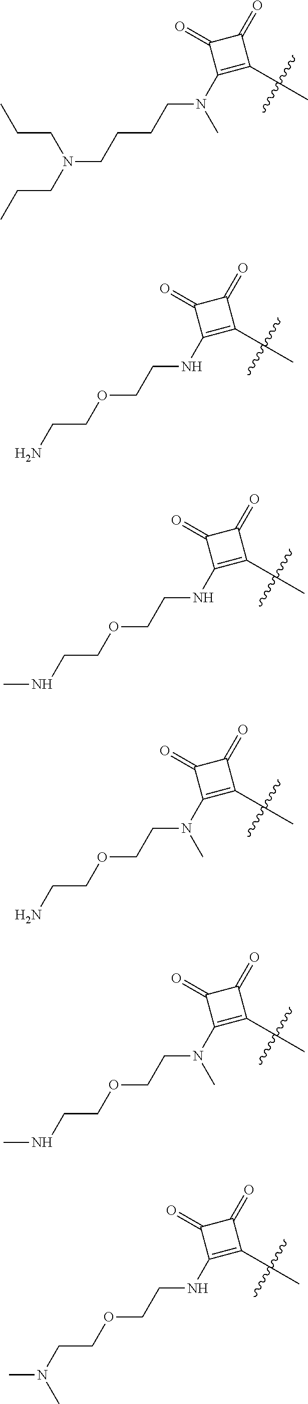 [SMILES: *c1c(N(C)CCCCN(CCC)CCC)c(=O)c1=O ; *c1c(NCCOCCN)c(=O)c1=O ; *c1c(NCCOCCNC)c(=O)c1=O ; *c1c(N(C)CCOCCN)c(=O)c1=O ; *c1c(N(C)CCOCCNC)c(=O)c1=O ; *c1c(NCCOCCN(C)C)c(=O)c1=O]

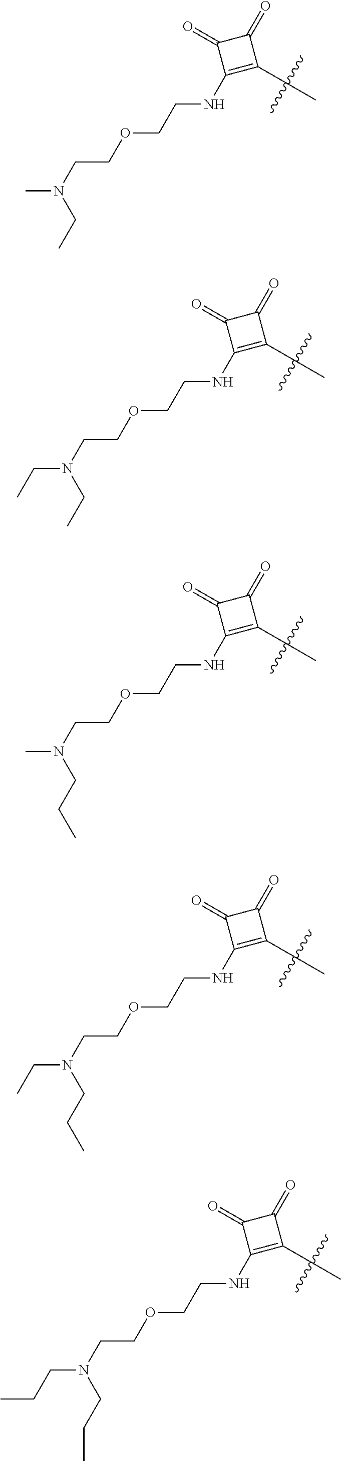 [SMILES: *c1c(NCCOCCN(C)CC)c(=O)c1=O ; *c1c(NCCOCCN(CC)CC)c(=O)c1=O ; *c1c(NCCOCCN(C)CCC)c(=O)c1=O ; *c1c(NCCOCCN(CC)CCC)c(=O)c1=O ; *c1c(NCCOCCN(CCC)CCC)c(=O)c1=O]

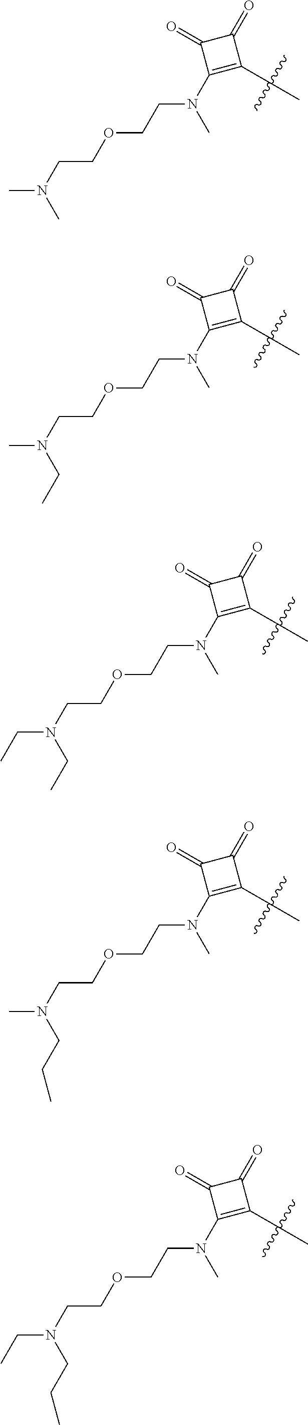 [SMILES: *c1c(N(C)CCOCCN(C)C)c(=O)c1=O ; *c1c(N(C)CCOCCN(C)CC)c(=O)c1=O ; *c1c(N(C)CCOCCN(CC)CC)c(=O)c1=O ; *c1c(N(C)CCOCCN(C)CCC)c(=O)c1=O ; *c1c(N(C)CCOCCN(CC)CCC)c(=O)c1=O]

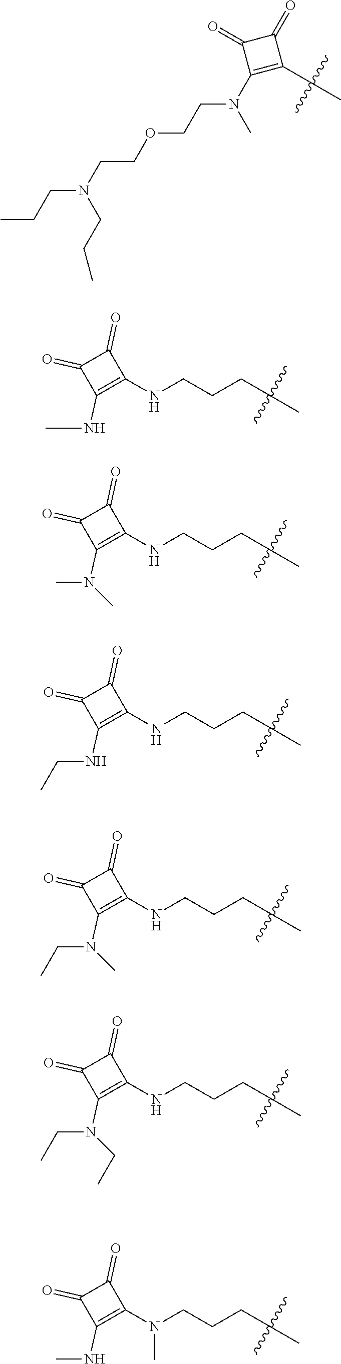

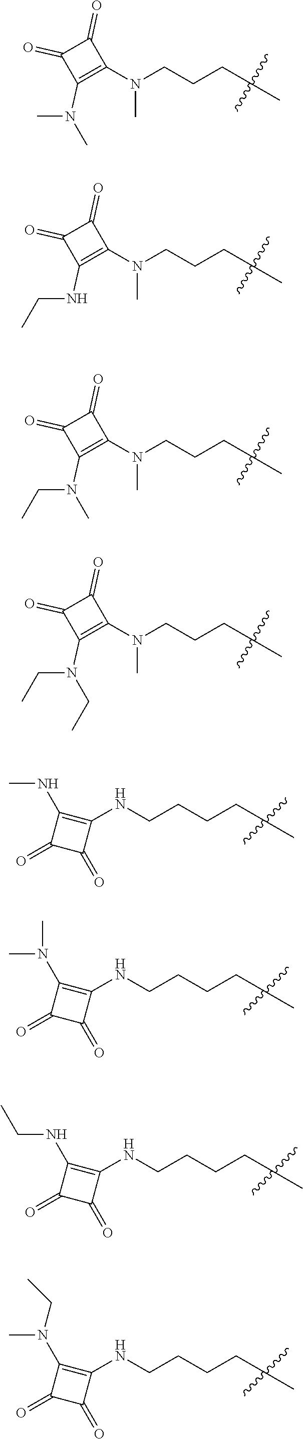

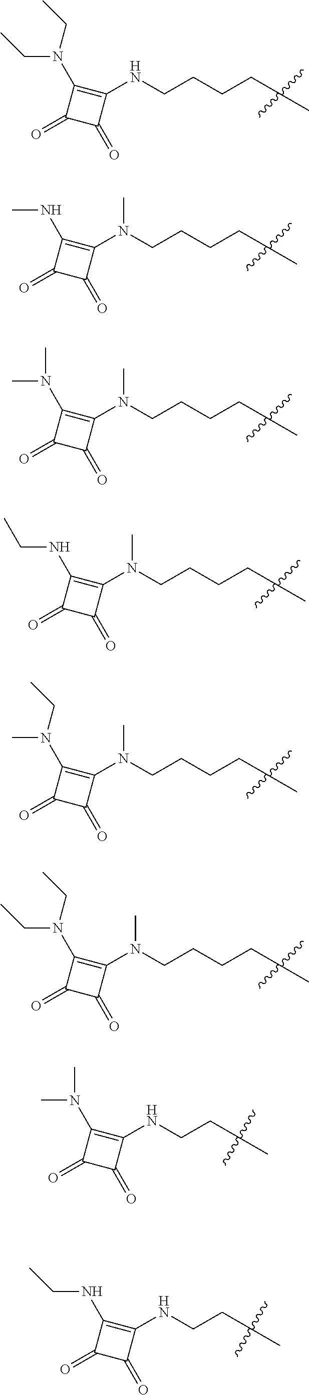

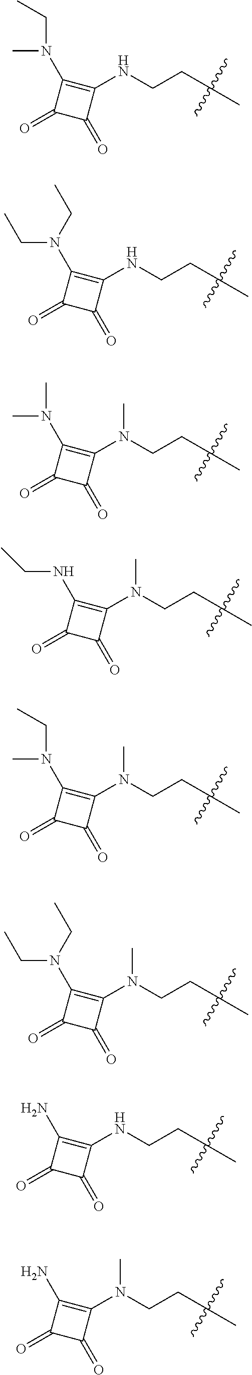

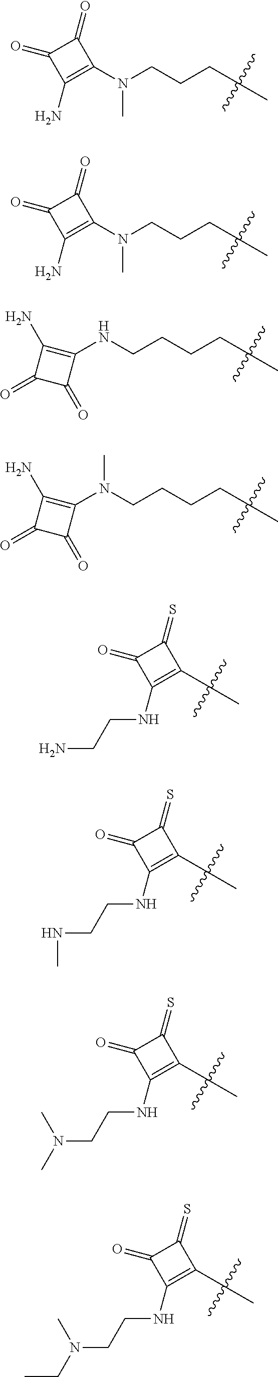

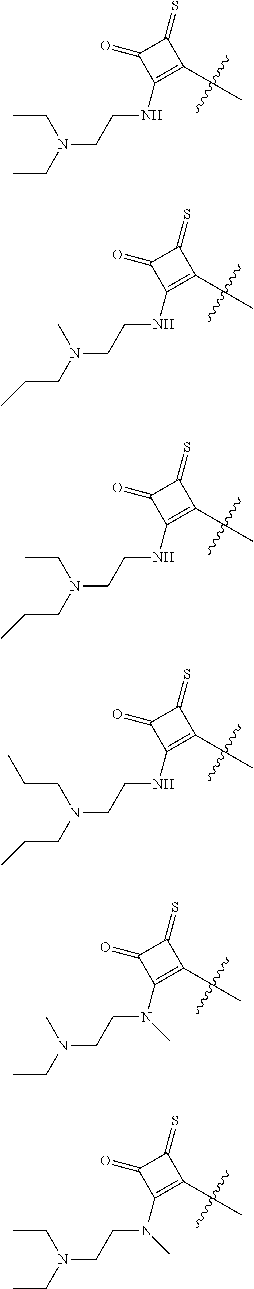 [SMILES: *c1c(NCCN(CC)CC)c(=O)c1=S ; *c1c(NCCN(C)CCC)c(=O)c1=S ; *c1c(NCCN(CC)CCC)c(=O)c1=S ; *c1c(NCCN(CCC)CCC)c(=O)c1=S ; *c1c(N(C)CCN(C)CC)c(=O)c1=S ; *c1c(N(C)CCN(CC)CC)c(=O)c1=S]

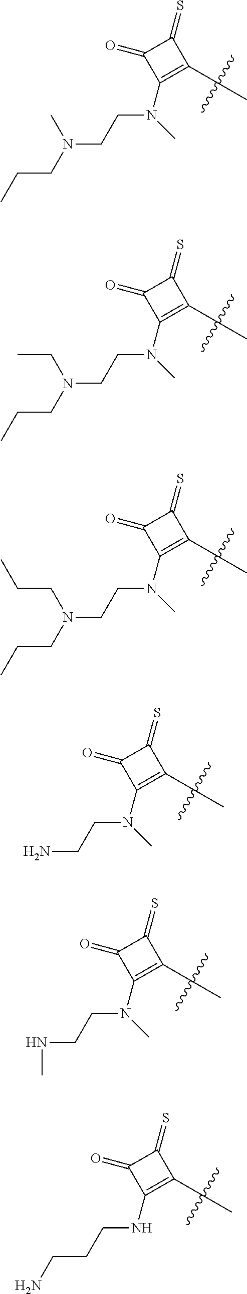 [SMILES: *c1c(N(C)CCN(C)CCC)c(=O)c1=S ; *c1c(N(C)CCN(CC)CCC)c(=O)c1=S ; *c1c(N(C)CCN(CCC)CCC)c(=O)c1=S ; *c1c(N(C)CCN)c(=O)c1=S ; *c1c(N(C)CCNC)c(=O)c1=S ; *c1c(NCCCN)c(=O)c1=S]

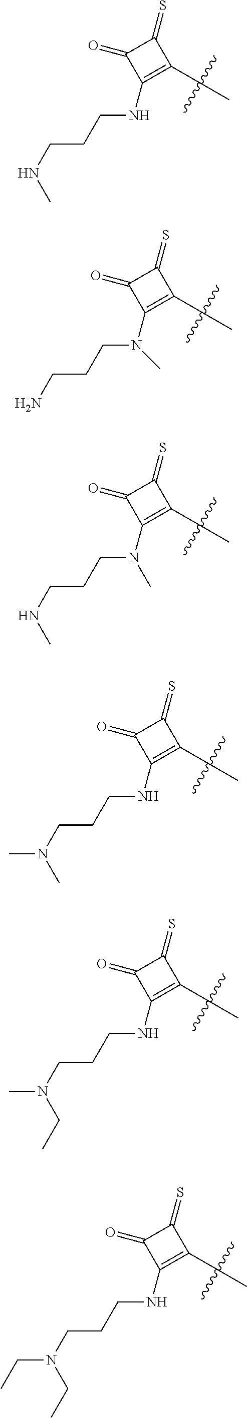 [SMILES: *c1c(NCCCNC)c(=O)c1=S ; *c1c(N(C)CCCN)c(=O)c1=S ; *c1c(N(C)CCCNC)c(=O)c1=S ; *c1c(NCCCN(C)C)c(=O)c1=S ; *c1c(NCCCN(C)CC)c(=O)c1=S ; *c1c(NCCCN(CC)CC)c(=O)c1=S]

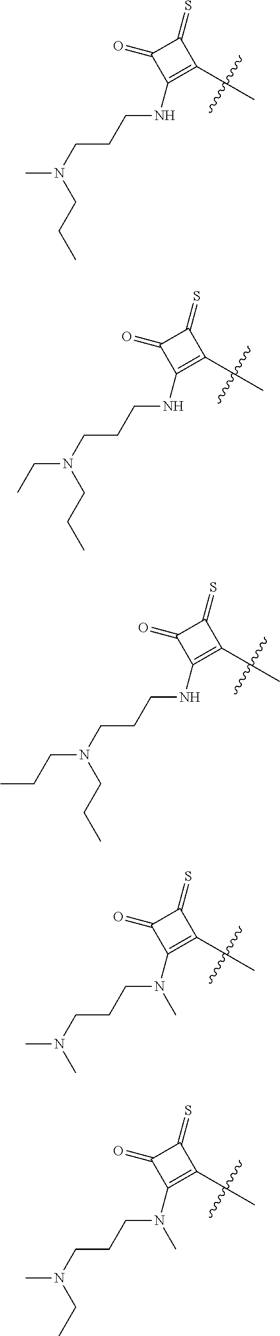 [SMILES: *c1c(NCCCN(C)CCC)c(=O)c1=S ; *c1c(NCCCN(CC)CCC)c(=O)c1=S ; *c1c(NCCCN(CCC)CCC)c(=O)c1=S ; *c1c(N(C)CCCN(C)C)c(=O)c1=S ; *c1c(N(C)CCCN(C)CC)c(=O)c1=S]

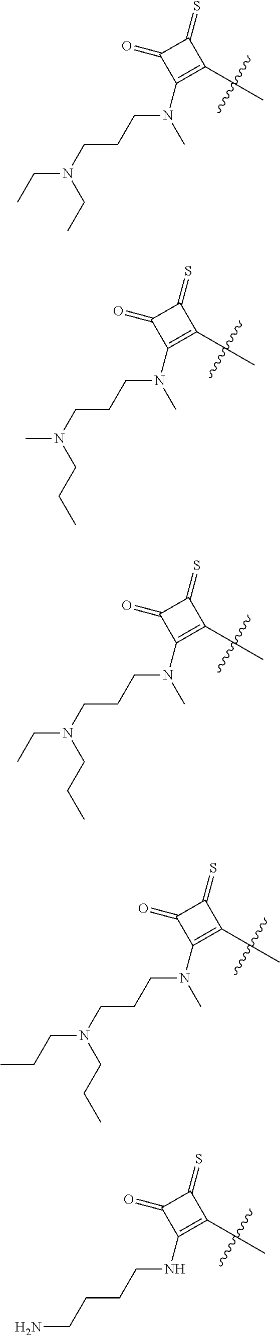 [SMILES: *c1c(N(C)CCCN(CC)CC)c(=O)c1=S ; *c1c(N(C)CCCN(C)CCC)c(=O)c1=S ; *c1c(N(C)CCCN(CC)CCC)c(=O)c1=S ; *c1c(N(C)CCCN(CCC)CCC)c(=O)c1=S ; *c1c(NCCCCN)c(=O)c1=S]

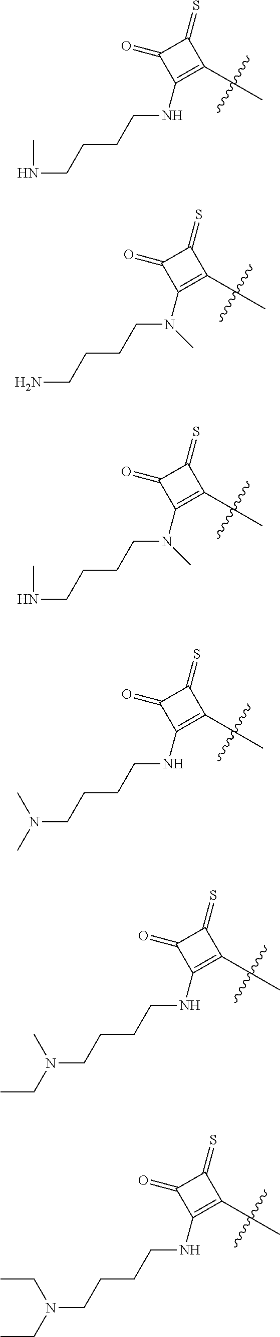 [SMILES: *c1c(NCCCCNC)c(=O)c1=S ; *c1c(N(C)CCCCN)c(=O)c1=S ; *c1c(N(C)CCCCNC)c(=O)c1=S ; *c1c(NCCCCN(C)C)c(=O)c1=S ; *c1c(NCCCCN(C)CC)c(=O)c1=S ; *c1c(NCCCCN(CC)CC)c(=O)c1=S]

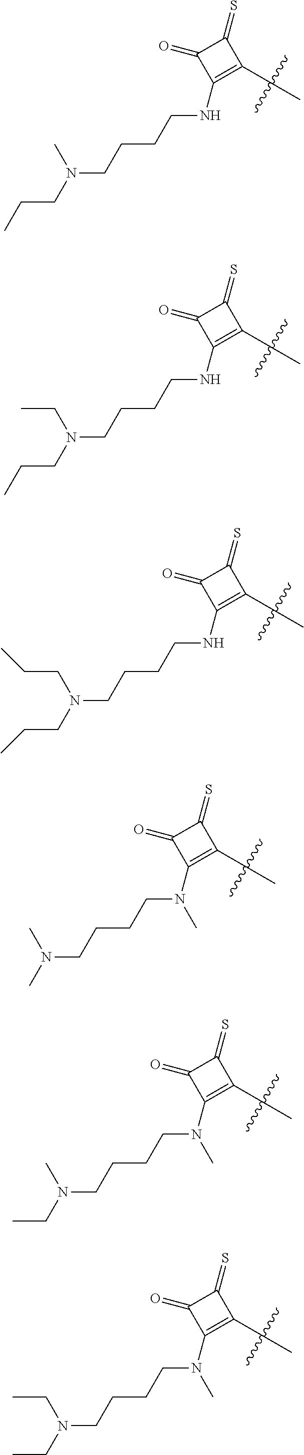 [SMILES: *c1c(NCCCCN(C)CCC)c(=O)c1=S ; *c1c(NCCCCN(CC)CCC)c(=O)c1=S ; *c1c(NCCCCN(CCC)CCC)c(=O)c1=S ; *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S]

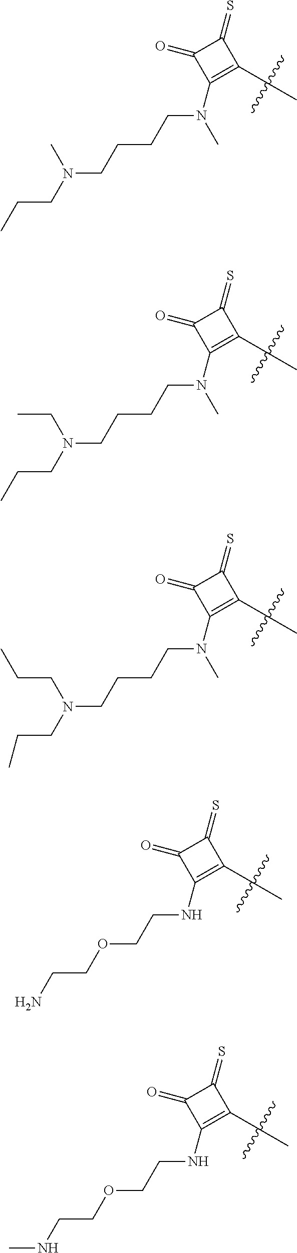 [SMILES: *c1c(N(C)CCCCN(C)CCC)c(=O)c1=S ; *c1c(N(C)CCCCN(CC)CCC)c(=O)c1=S ; *c1c(N(C)CCCCN(CCC)CCC)c(=O)c1=S ; *c1c(NCCOCCN)c(=O)c1=S ; *c1c(NCCOCCNC)c(=O)c1=S]

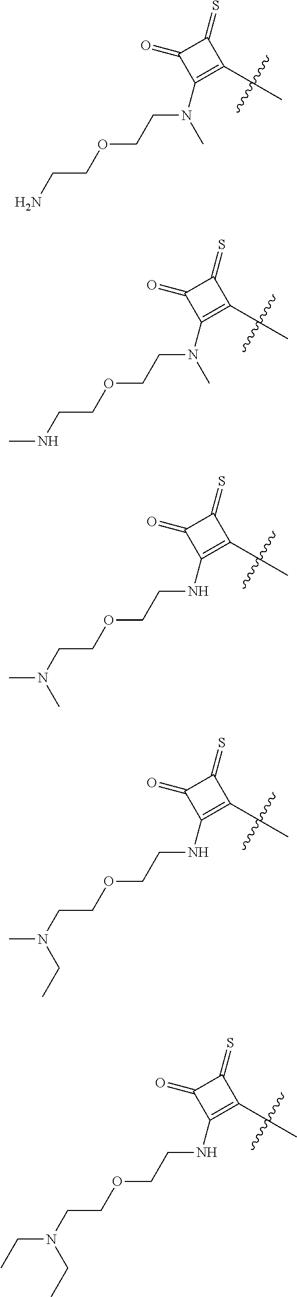 [SMILES: *c1c(N(C)CCOCCN)c(=O)c1=S ; *c1c(N(C)CCOCCNC)c(=O)c1=S ; *c1c(NCCOCCN(C)C)c(=O)c1=S ; *c1c(NCCOCCN(C)CC)c(=O)c1=S ; *c1c(NCCOCCN(CC)CC)c(=O)c1=S]

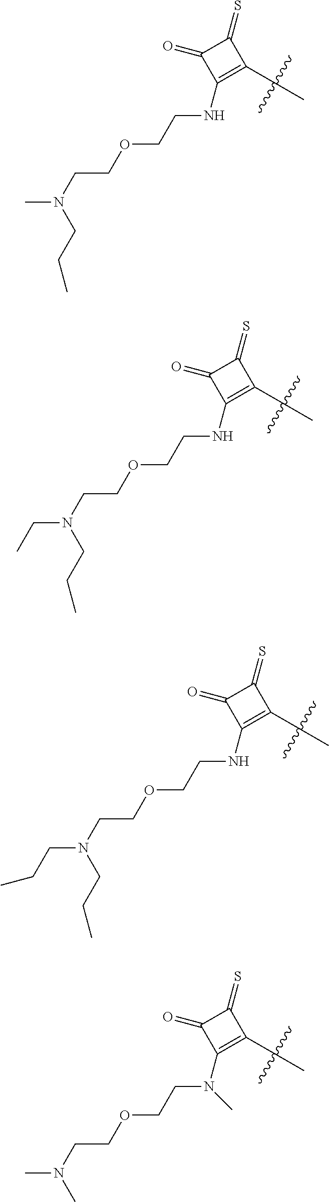 [SMILES: *c1c(NCCOCCN(C)CCC)c(=O)c1=S ; *c1c(NCCOCCN(CC)CCC)c(=O)c1=S ; *c1c(NCCOCCN(CCC)CCC)c(=O)c1=S ; *c1c(N(C)CCOCCN(C)C)c(=O)c1=S]

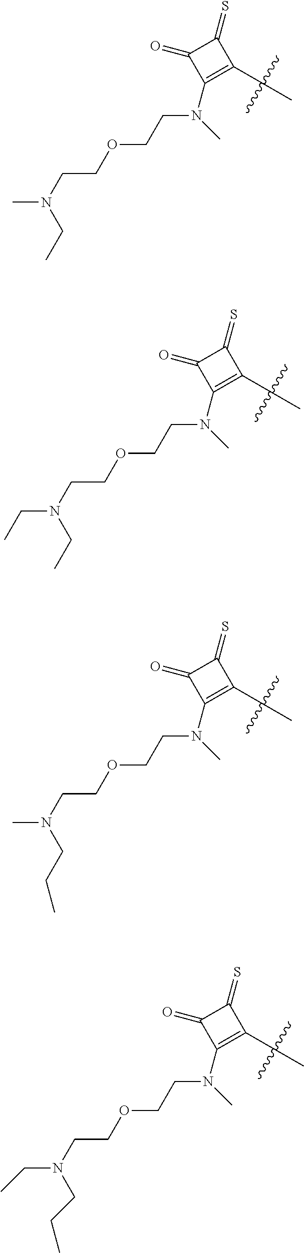 [SMILES: *c1c(N(C)CCOCCN(C)CC)c(=O)c1=S ; *c1c(N(C)CCOCCN(CC)CC)c(=O)c1=S ; *c1c(N(C)CCOCCN(C)CCC)c(=O)c1=S ; *c1c(N(C)CCOCCN(CC)CCC)c(=O)c1=S]

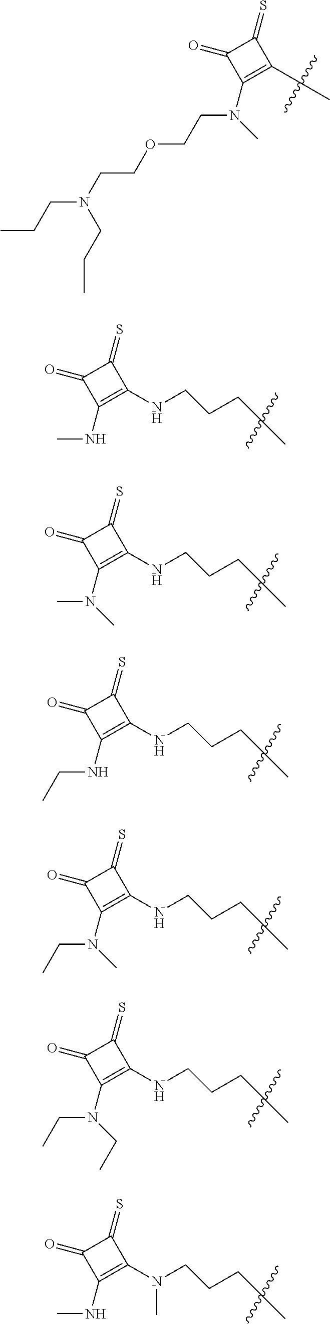

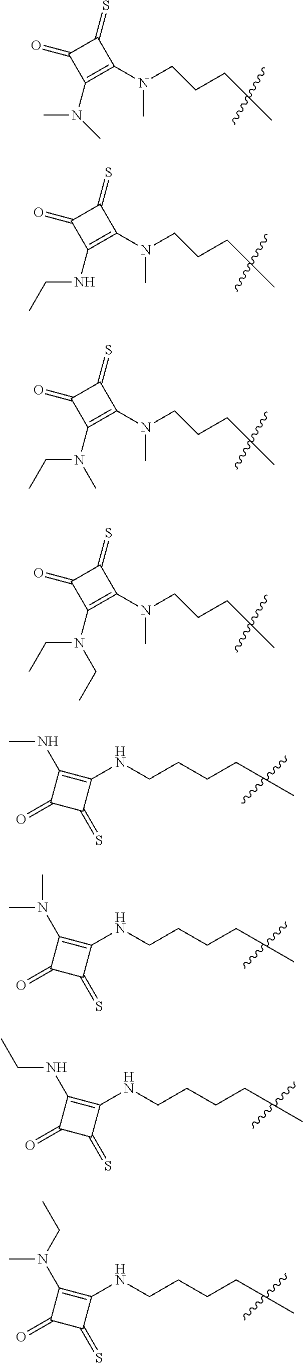

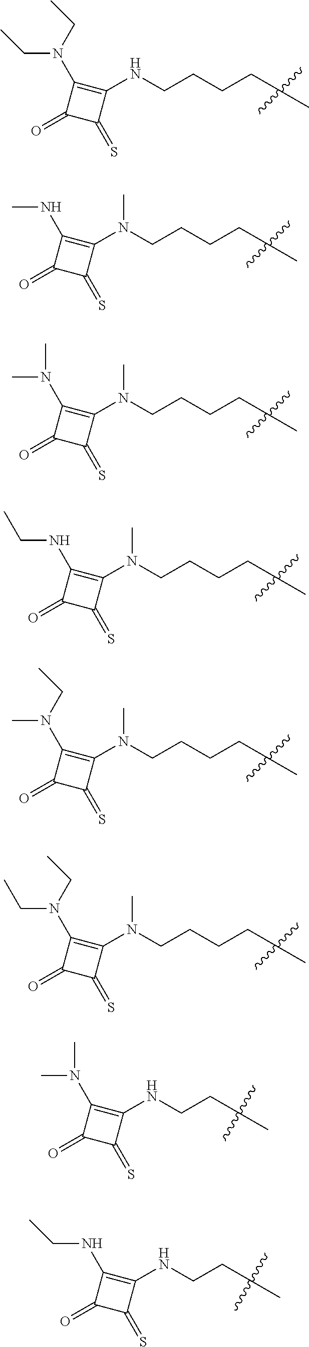

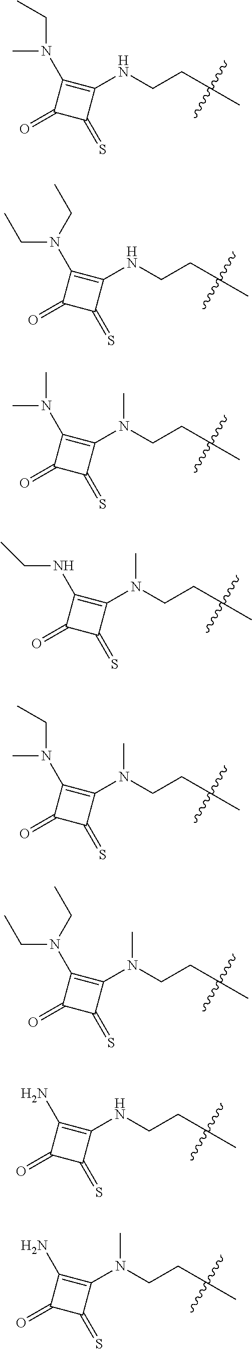

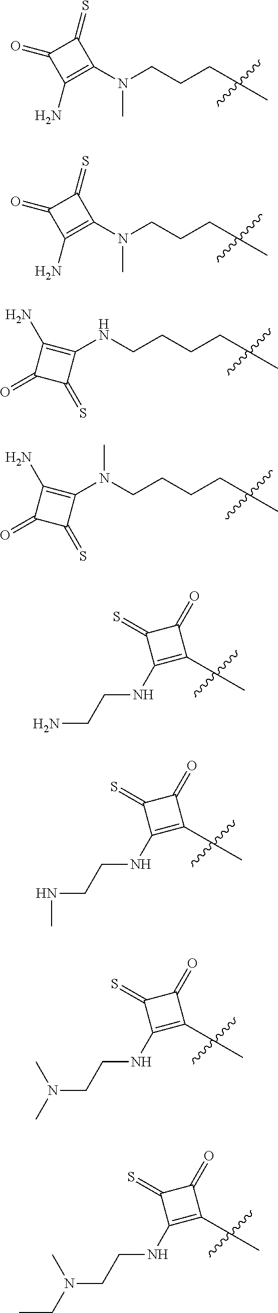

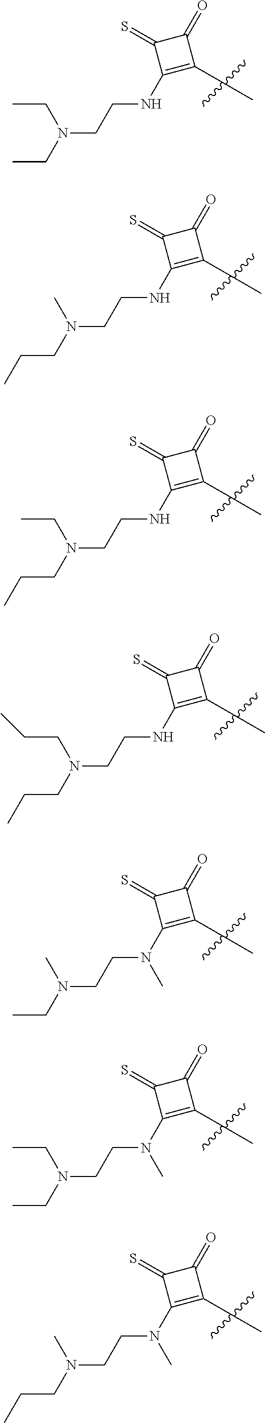

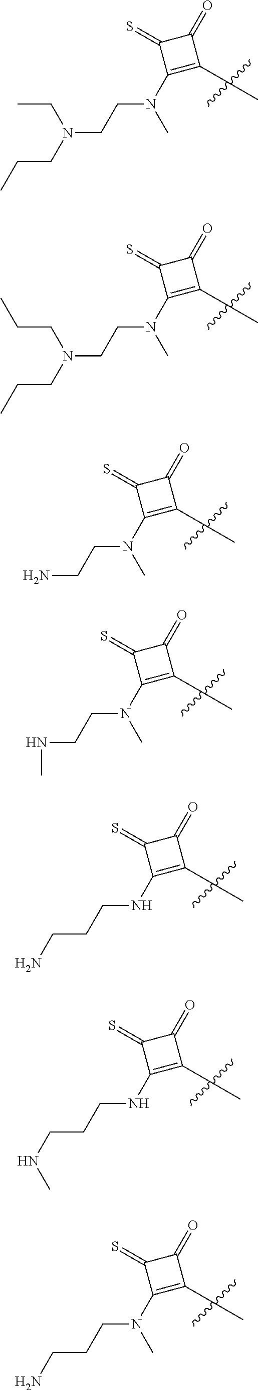

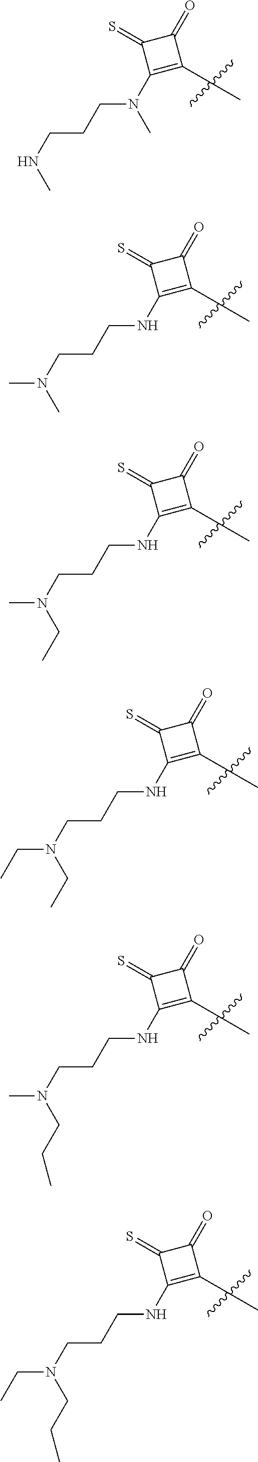 [SMILES: *c1c(N(C)CCCNC)c(=S)c1=O ; *c1c(NCCCN(C)C)c(=S)c1=O ; *c1c(NCCCN(C)CC)c(=S)c1=O ; *c1c(NCCCN(CC)CC)c(=S)c1=O ; *c1c(NCCCN(C)CCC)c(=S)c1=O ; *c1c(NCCCN(CC)CCC)c(=S)c1=O]

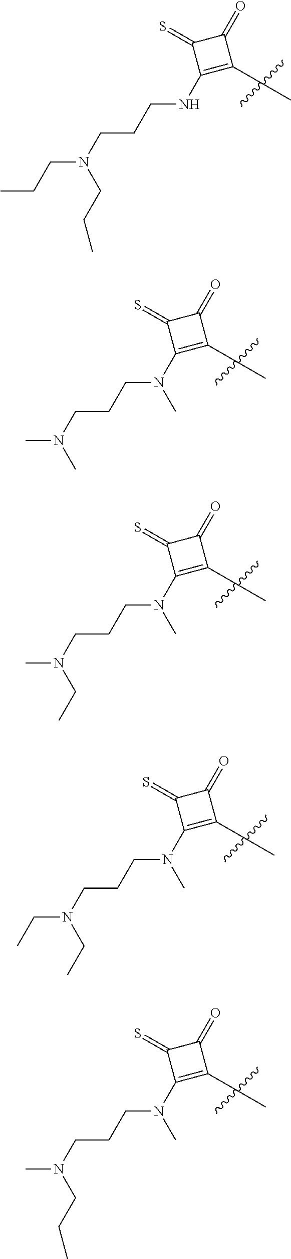 [SMILES: *c1c(NCCCN(CCC)CCC)c(=S)c1=O ; *c1c(N(C)CCCN(C)C)c(=S)c1=O ; *c1c(N(C)CCCN(C)CC)c(=S)c1=O ; *c1c(N(C)CCCN(CC)CC)c(=S)c1=O ; *c1c(N(C)CCCN(C)CCC)c(=S)c1=O]

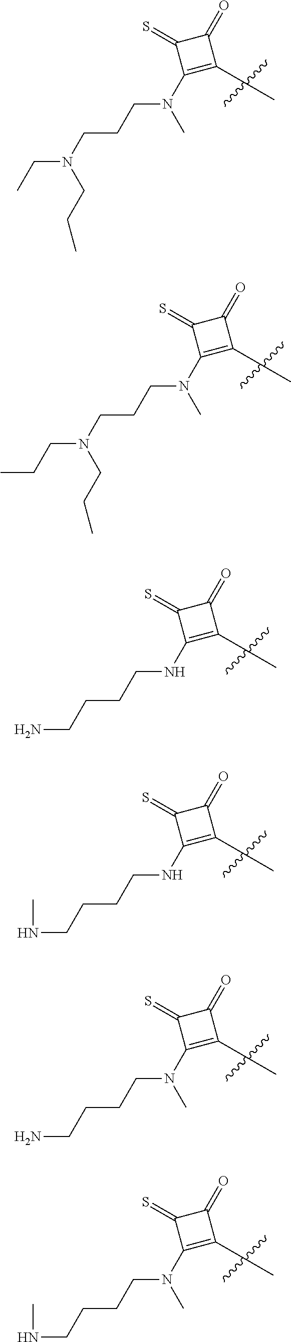 [SMILES: *c1c(N(C)CCCN(CC)CCC)c(=S)c1=O ; *c1c(N(C)CCCN(CCC)CCC)c(=S)c1=O ; *c1c(NCCCCN)c(=S)c1=O ; *c1c(NCCCCNC)c(=S)c1=O ; *c1c(N(C)CCCCN)c(=S)c1=O ; *c1c(N(C)CCCCNC)c(=S)c1=O]

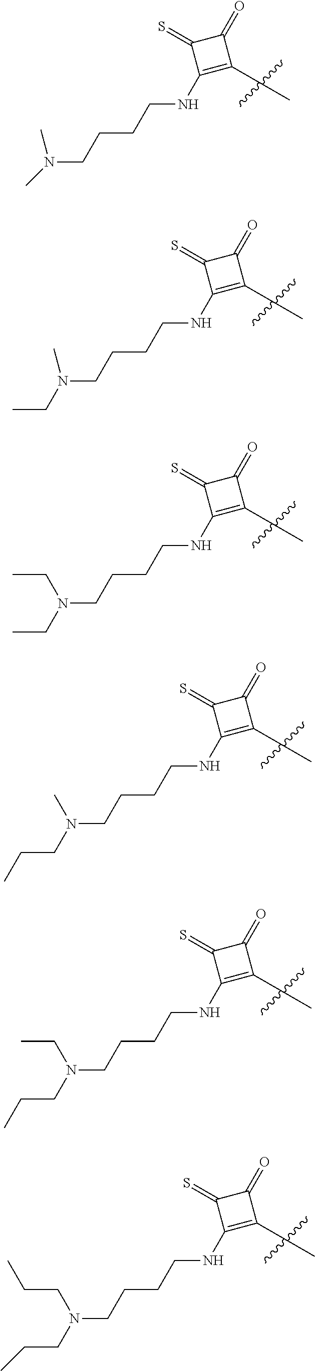 [SMILES: *c1c(NCCCCN(C)C)c(=S)c1=O ; *c1c(NCCCCN(C)CC)c(=S)c1=O ; *c1c(NCCCCN(CC)CC)c(=S)c1=O ; *c1c(NCCCCN(C)CCC)c(=S)c1=O ; *c1c(NCCCCN(CC)CCC)c(=S)c1=O ; *c1c(NCCCCN(CCC)CCC)c(=S)c1=O]

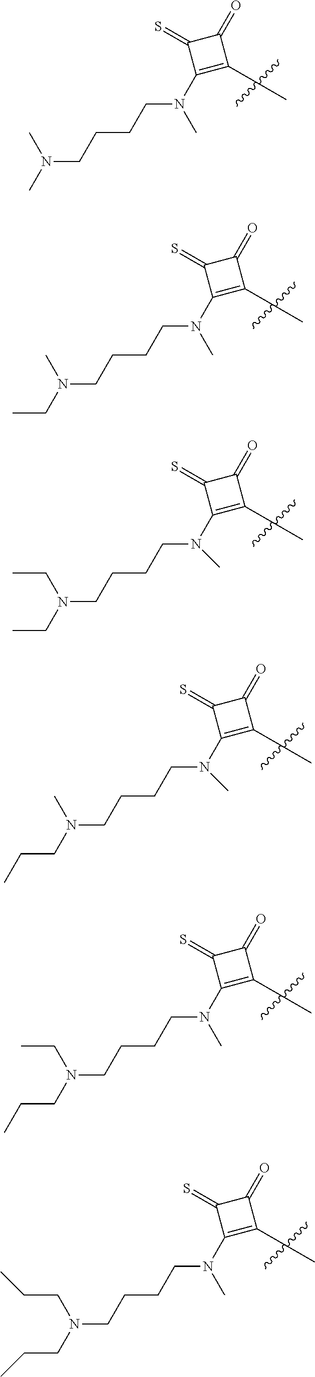 [SMILES: *c1c(N(C)CCCCN(C)C)c(=S)c1=O ; *c1c(N(C)CCCCN(C)CC)c(=S)c1=O ; *c1c(N(C)CCCCN(CC)CC)c(=S)c1=O ; *c1c(N(C)CCCCN(C)CCC)c(=S)c1=O ; *c1c(N(C)CCCCN(CC)CCC)c(=S)c1=O ; *c1c(N(C)CCCCN(CCC)CCC)c(=S)c1=O]

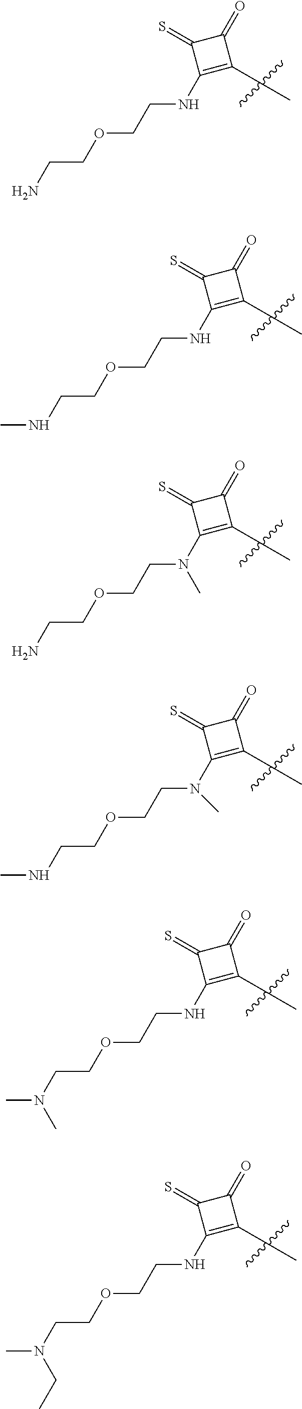 [SMILES: *c1c(NCCOCCN)c(=S)c1=O ; *c1c(NCCOCCNC)c(=S)c1=O ; *c1c(N(C)CCOCCN)c(=S)c1=O ; *c1c(N(C)CCOCCNC)c(=S)c1=O ; *c1c(NCCOCCN(C)C)c(=S)c1=O ; *c1c(NCCOCCN(C)CC)c(=S)c1=O]

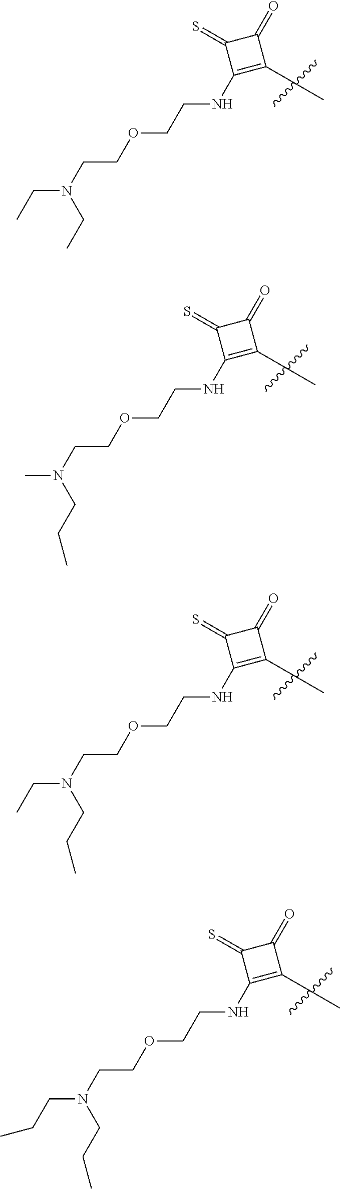 [SMILES: *c1c(NCCOCCN(CC)CC)c(=S)c1=O ; *c1c(NCCOCCN(C)CCC)c(=S)c1=O ; *c1c(NCCOCCN(CC)CCC)c(=S)c1=O ; *c1c(NCCOCCN(CCC)CCC)c(=S)c1=O]

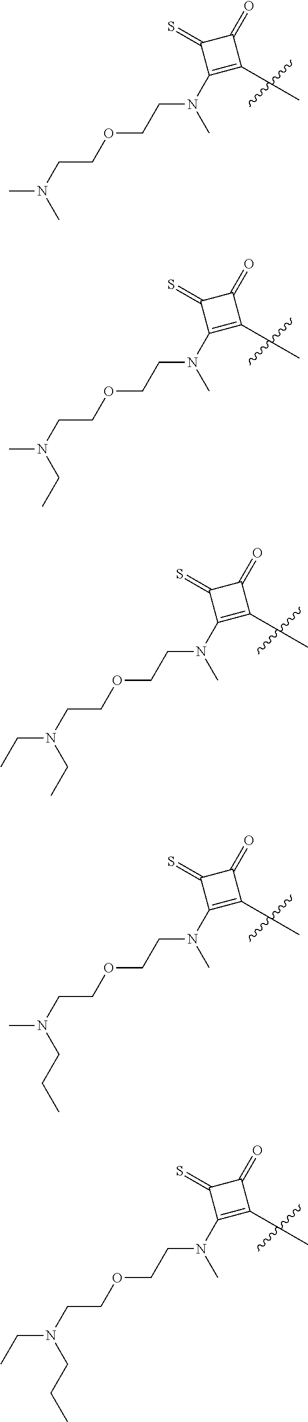 [SMILES: *c1c(N(C)CCOCCN(C)C)c(=S)c1=O ; *c1c(N(C)CCOCCN(C)CC)c(=S)c1=O ; *c1c(N(C)CCOCCN(CC)CC)c(=S)c1=O ; *c1c(N(C)CCOCCN(C)CCC)c(=S)c1=O ; *c1c(N(C)CCOCCN(CC)CCC)c(=S)c1=O]

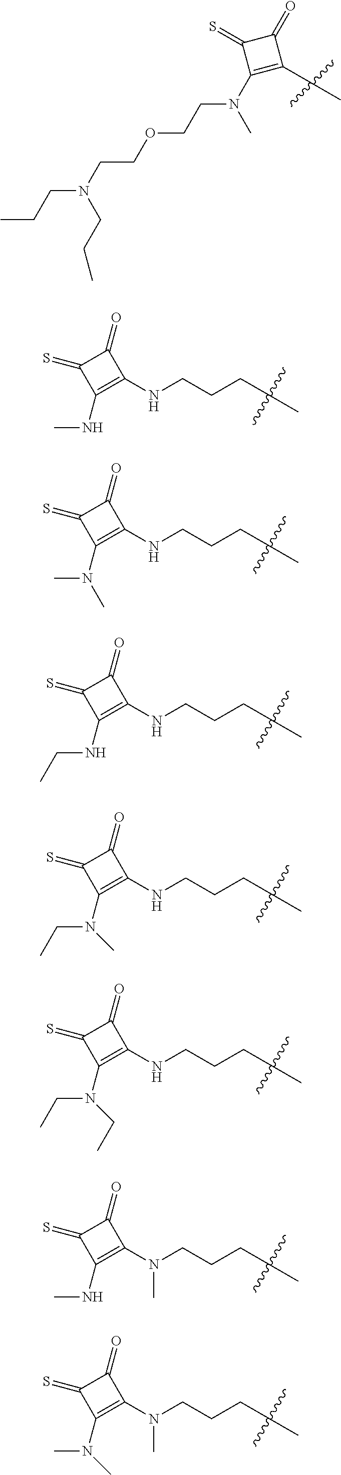

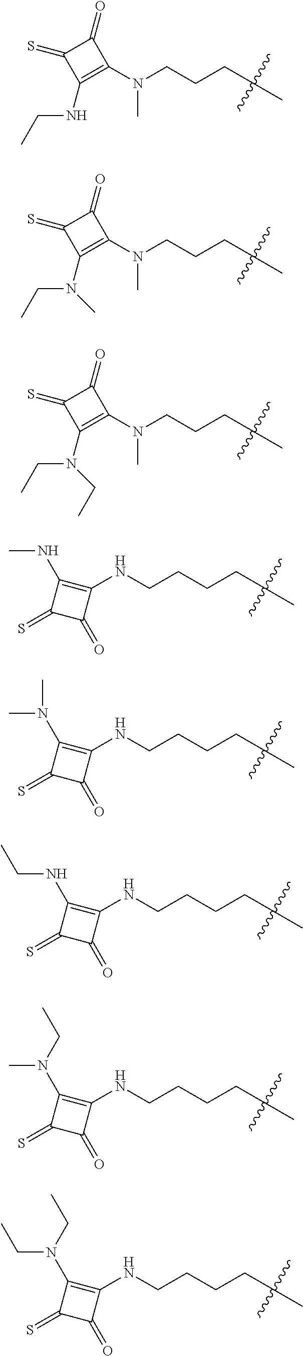

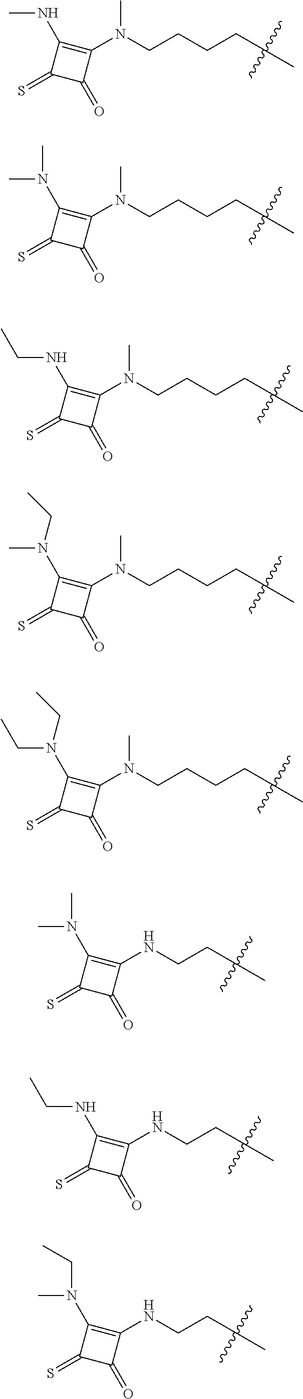

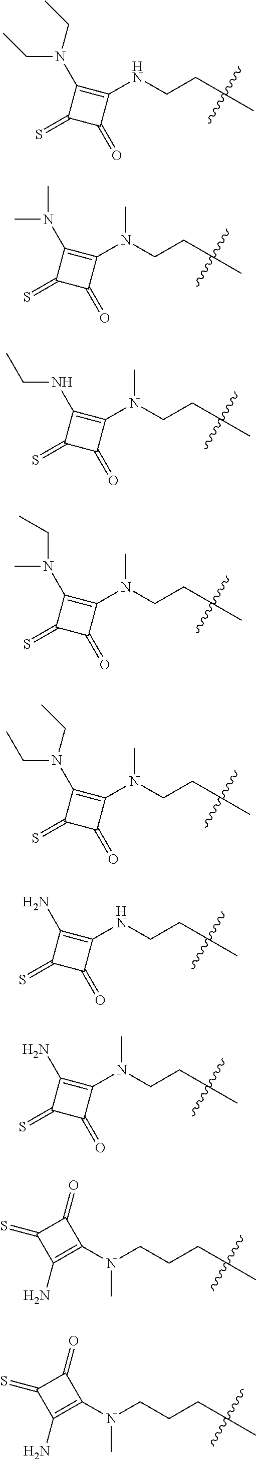

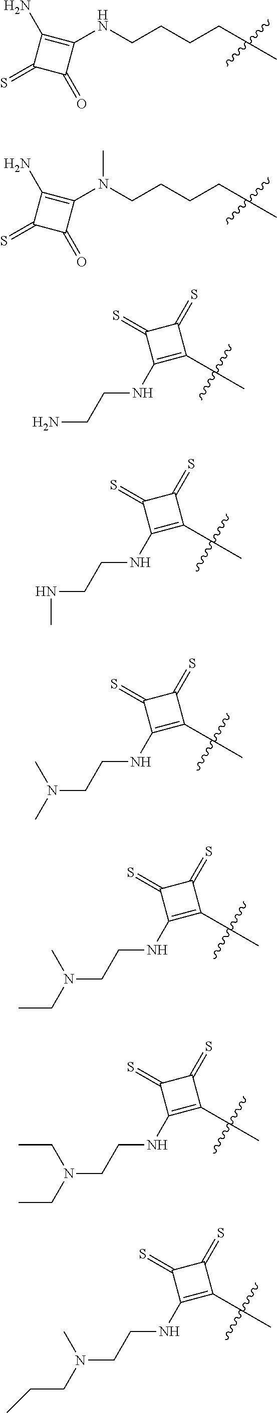

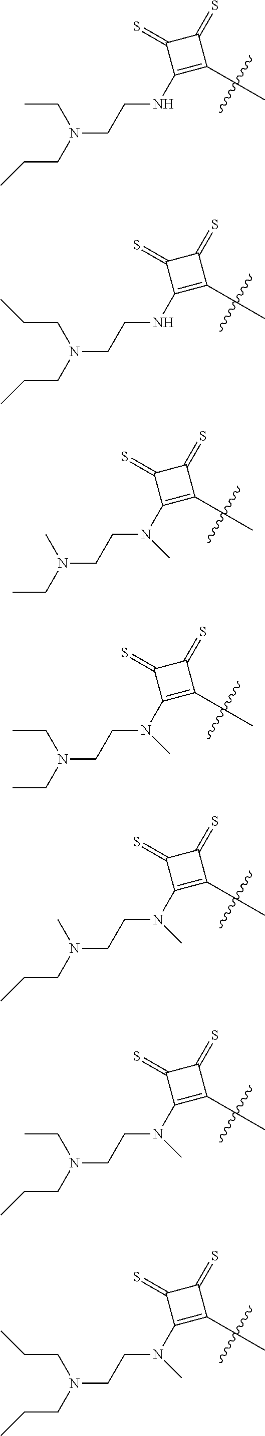

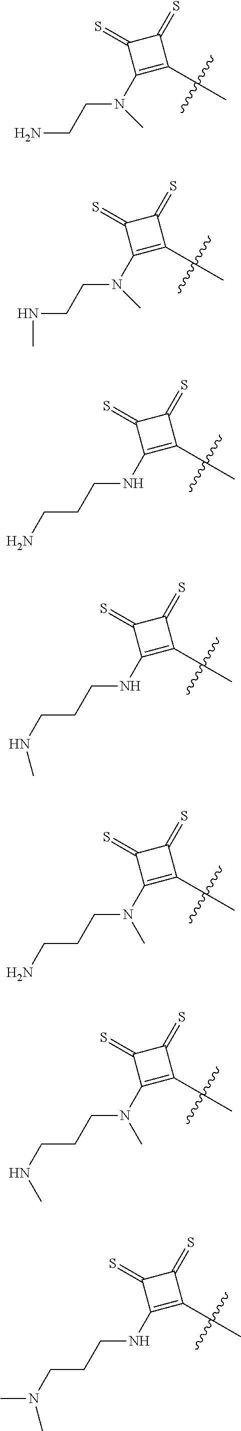

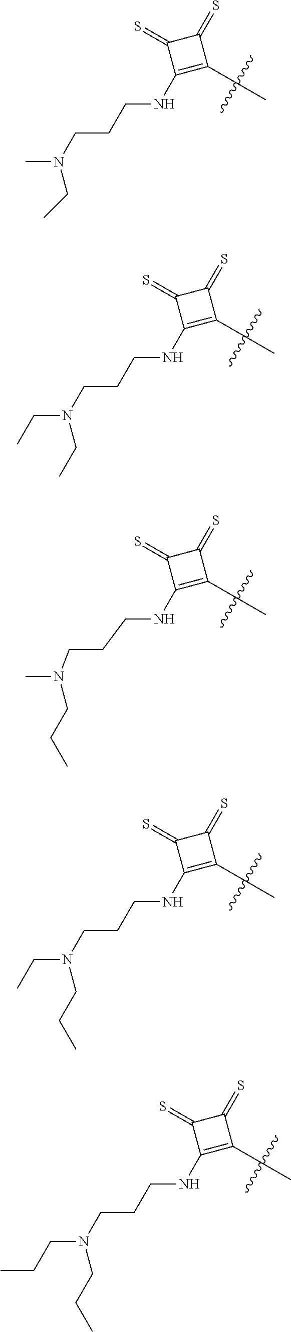 [SMILES: *c1c(NCCCN(C)CC)c(=S)c1=S ; *c1c(NCCCN(CC)CC)c(=S)c1=S ; *c1c(NCCCN(C)CCC)c(=S)c1=S ; *c1c(NCCCN(CC)CCC)c(=S)c1=S ; *c1c(NCCCN(CCC)CCC)c(=S)c1=S]

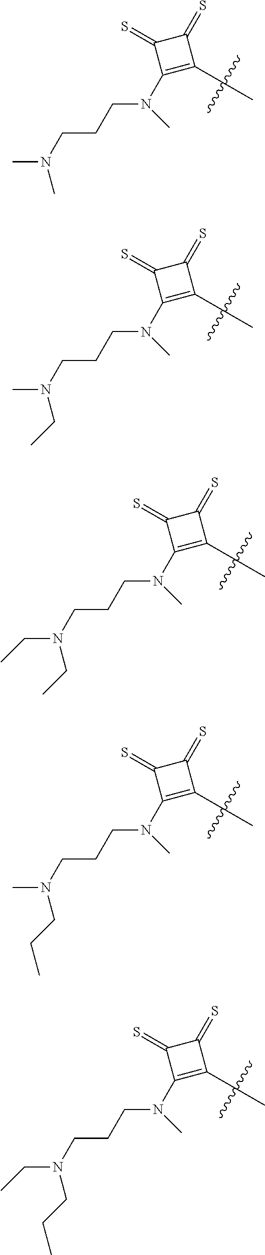 [SMILES: *c1c(N(C)CCCN(C)C)c(=S)c1=S ; *c1c(N(C)CCCN(C)CC)c(=S)c1=S ; *c1c(N(C)CCCN(CC)CC)c(=S)c1=S ; *c1c(N(C)CCCN(C)CCC)c(=S)c1=S ; *c1c(N(C)CCCN(CC)CCC)c(=S)c1=S]

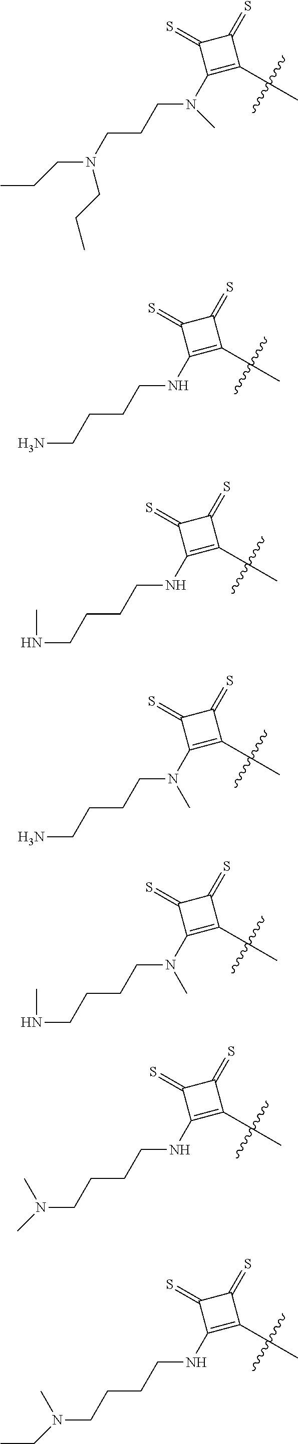

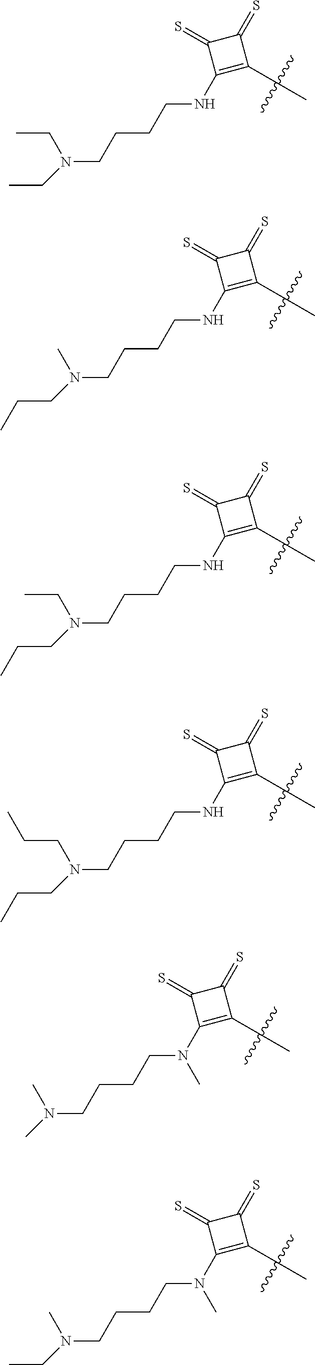 [SMILES: *c1c(NCCCCN(CC)CC)c(=S)c1=S ; *c1c(NCCCCN(C)CCC)c(=S)c1=S ; *c1c(NCCCCN(CC)CCC)c(=S)c1=S ; *c1c(NCCCCN(CCC)CCC)c(=S)c1=S ; *c1c(N(C)CCCCN(C)C)c(=S)c1=S ; *c1c(N(C)CCCCN(C)CC)c(=S)c1=S]

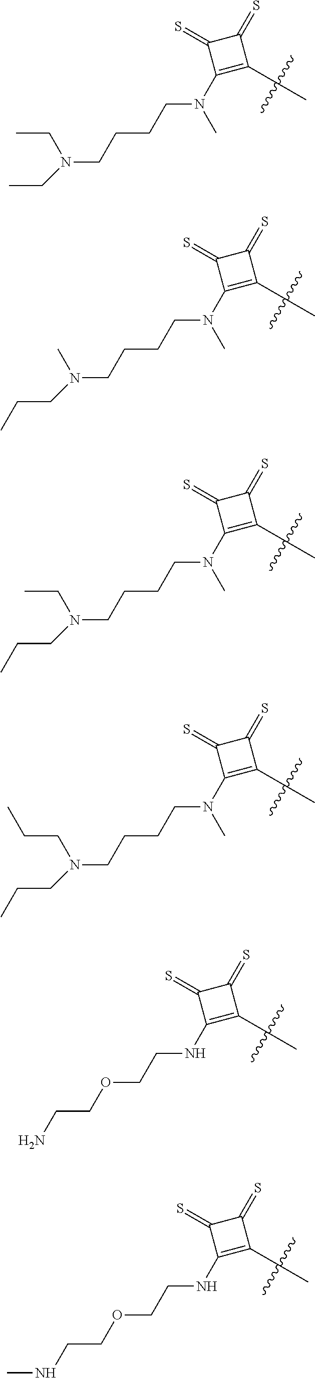 [SMILES: *c1c(N(C)CCCCN(CC)CC)c(=S)c1=S ; *c1c(N(C)CCCCN(C)CCC)c(=S)c1=S ; *c1c(N(C)CCCCN(CC)CCC)c(=S)c1=S ; *c1c(N(C)CCCCN(CCC)CCC)c(=S)c1=S ; *c1c(NCCOCCN)c(=S)c1=S ; *c1c(NCCOCCNC)c(=S)c1=S]

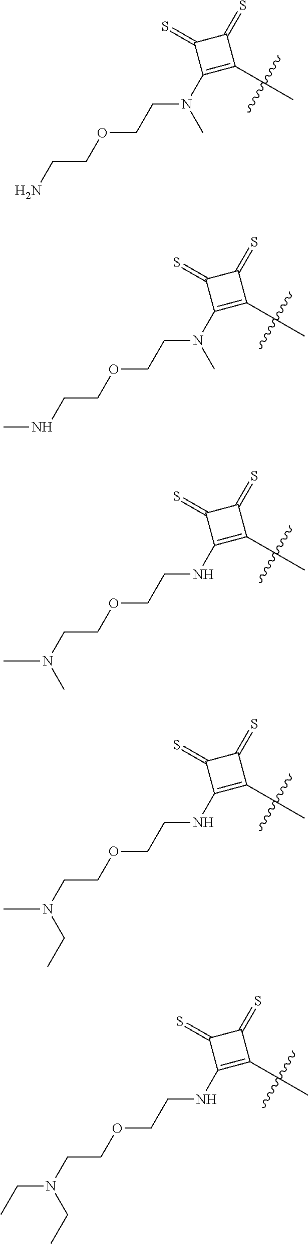 [SMILES: *c1c(N(C)CCOCCN)c(=S)c1=S ; *c1c(N(C)CCOCCNC)c(=S)c1=S ; *c1c(NCCOCCN(C)C)c(=S)c1=S ; *c1c(NCCOCCN(C)CC)c(=S)c1=S ; *c1c(NCCOCCN(CC)CC)c(=S)c1=S]

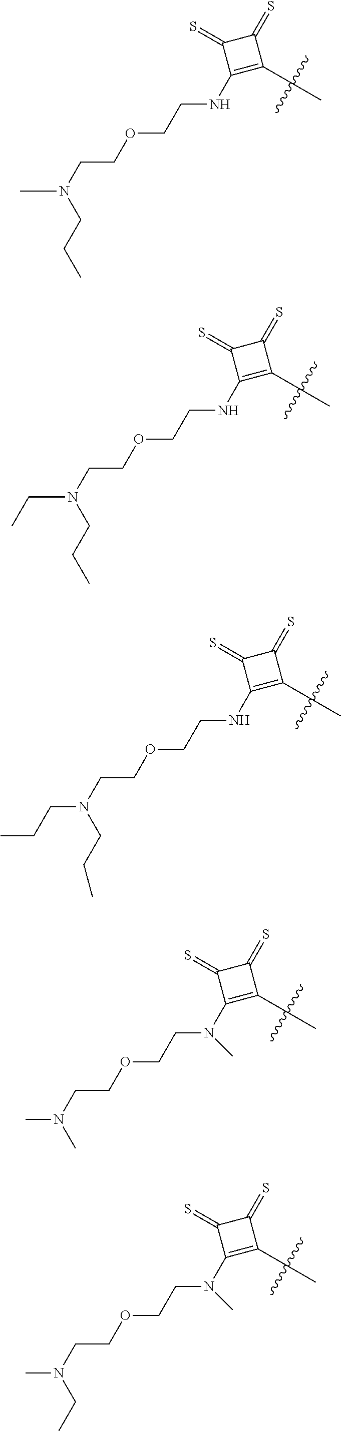 [SMILES: *c1c(NCCOCCN(C)CCC)c(=S)c1=S ; *c1c(NCCOCCN(CC)CCC)c(=S)c1=S ; *c1c(NCCOCCN(CCC)CCC)c(=S)c1=S ; *c1c(N(C)CCOCCN(C)C)c(=S)c1=S ; *c1c(N(C)CCOCCN(C)CC)c(=S)c1=S]

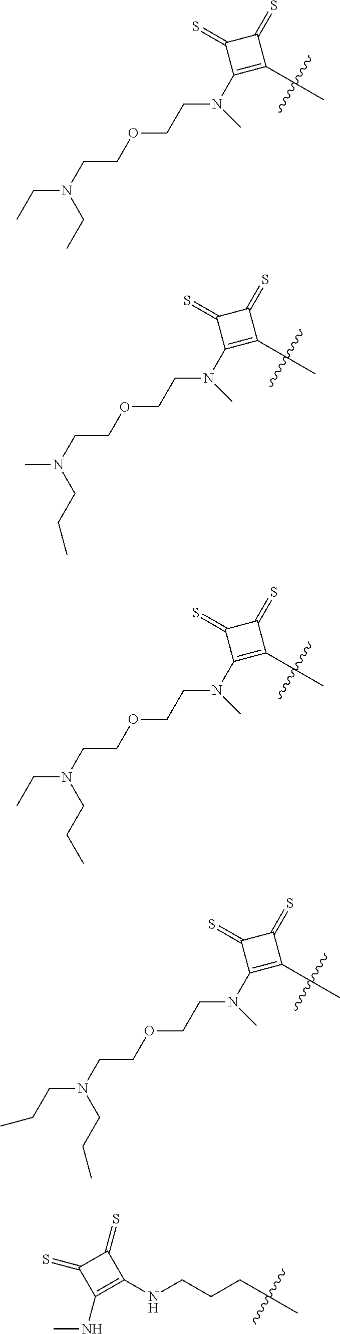 [SMILES: *c1c(N(C)CCOCCN(CC)CC)c(=S)c1=S ; *c1c(N(C)CCOCCN(C)CCC)c(=S)c1=S ; *c1c(N(C)CCOCCN(CC)CCC)c(=S)c1=S ; *c1c(N(C)CCOCCN(CCC)CCC)c(=S)c1=S ; *CCCNc1c(NC)c(=S)c1=S]

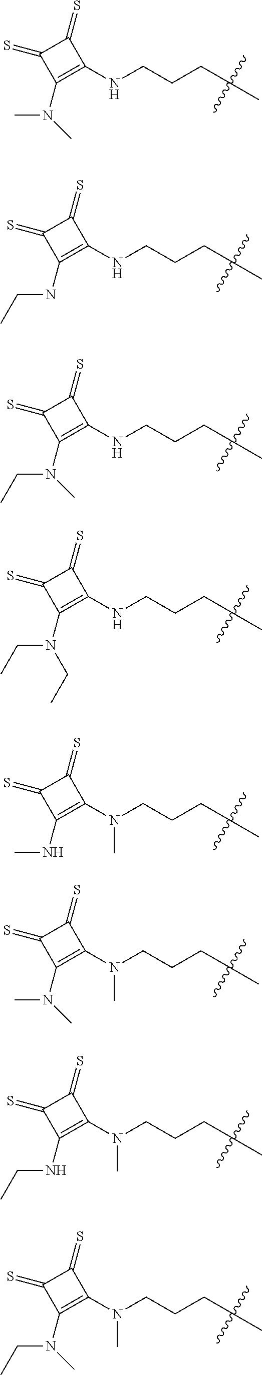

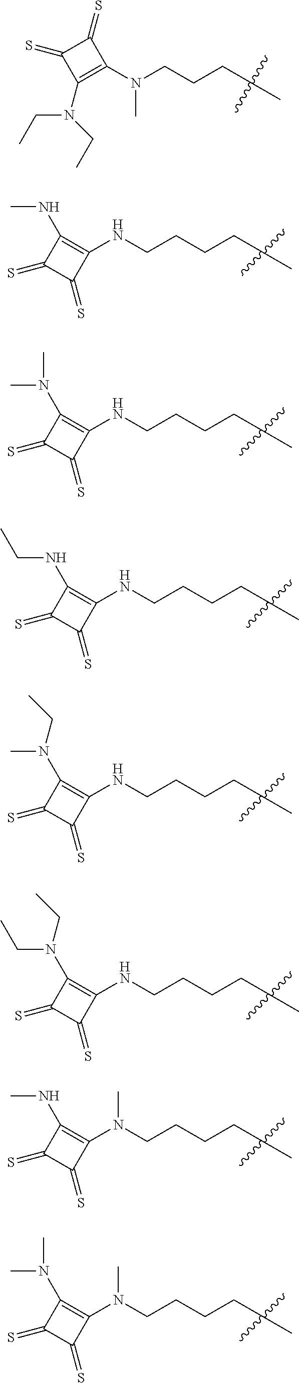

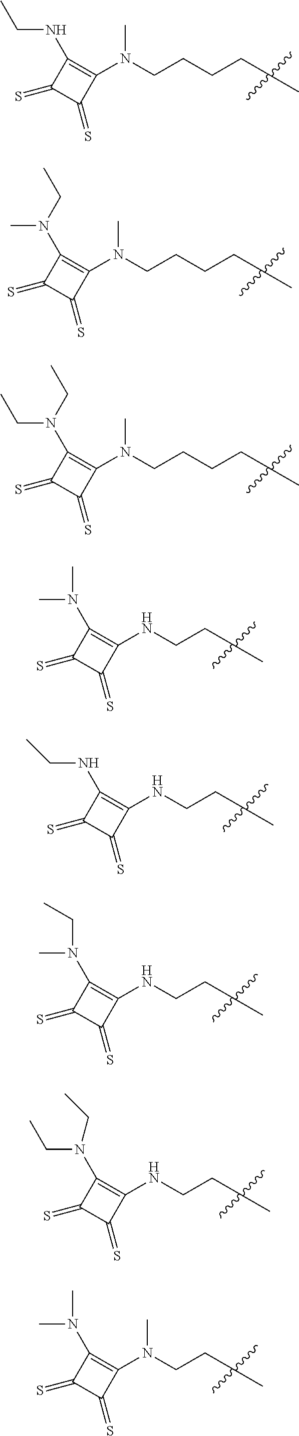

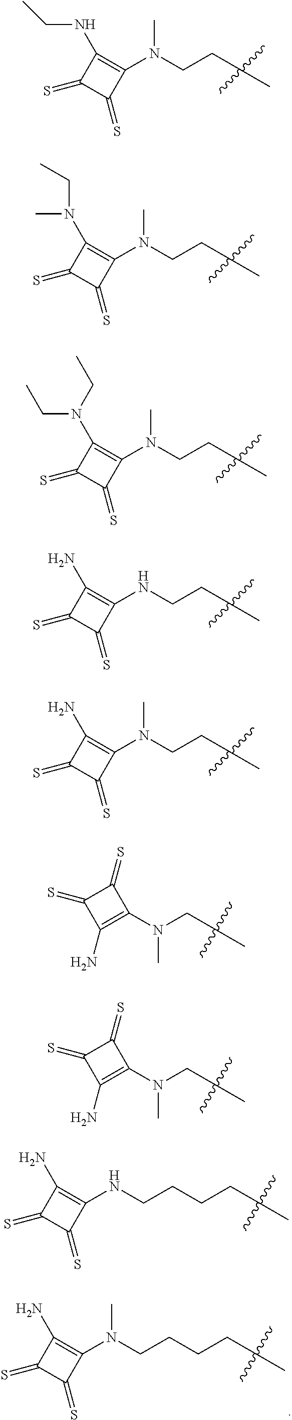

In some embodiments, R⁴ is selected from any of the following groups:

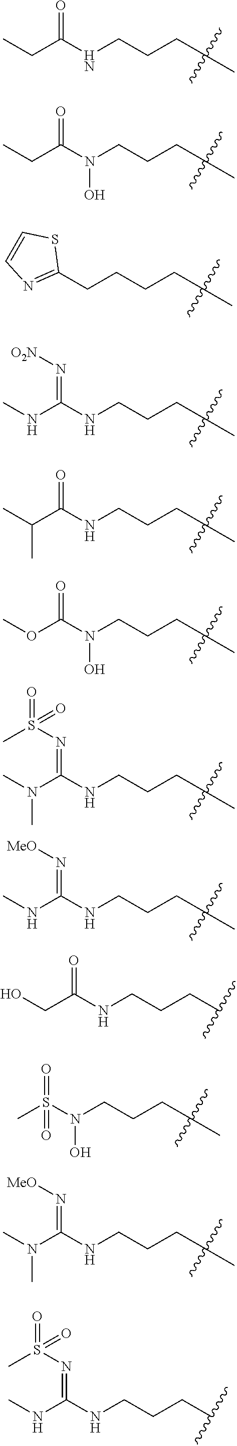

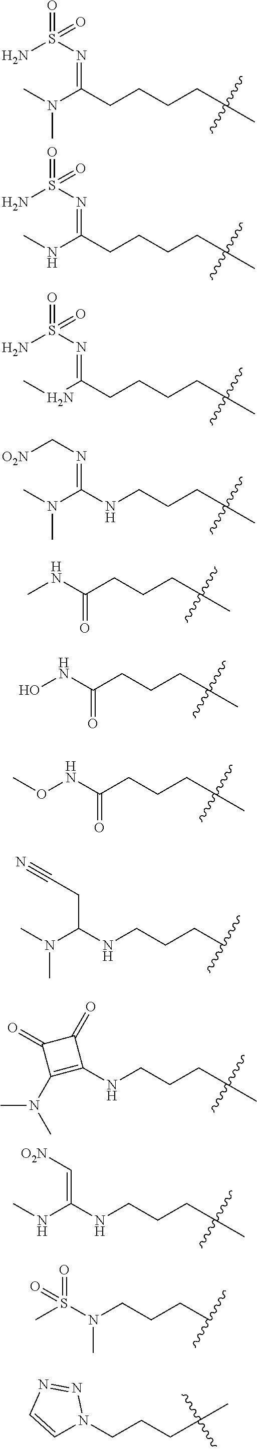

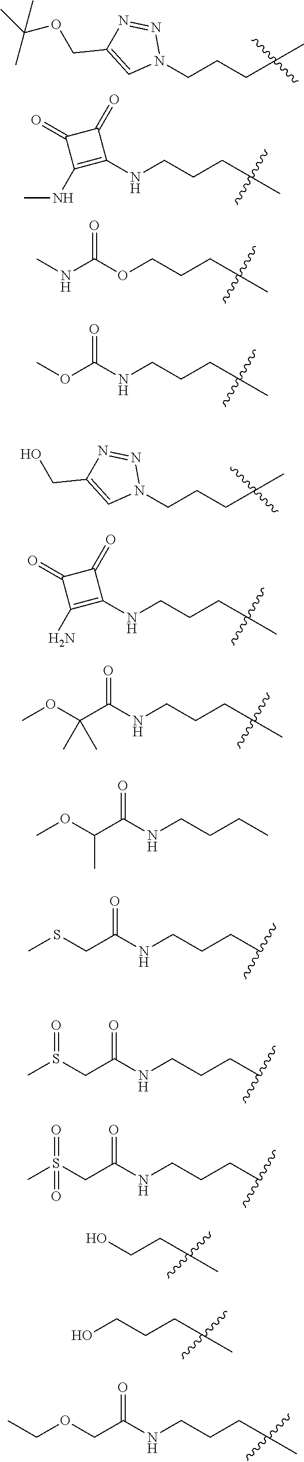

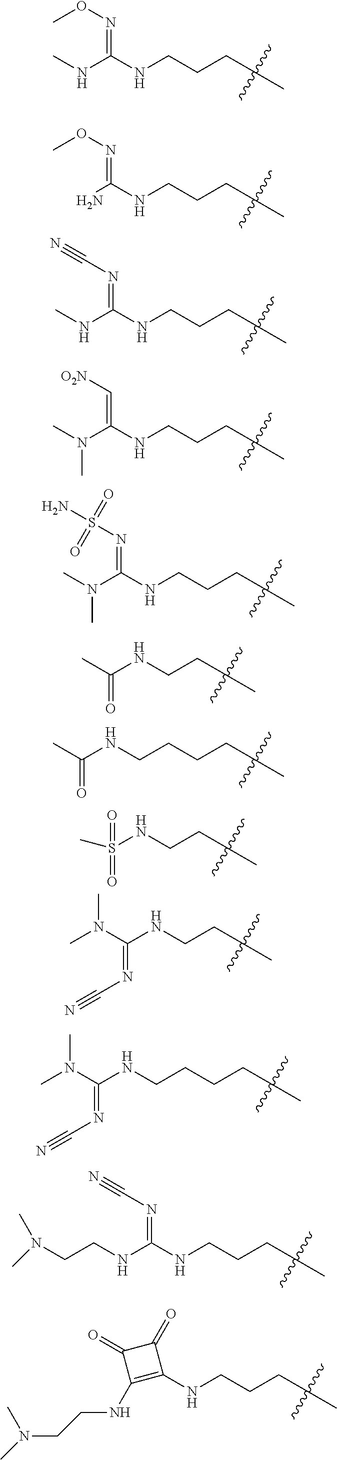

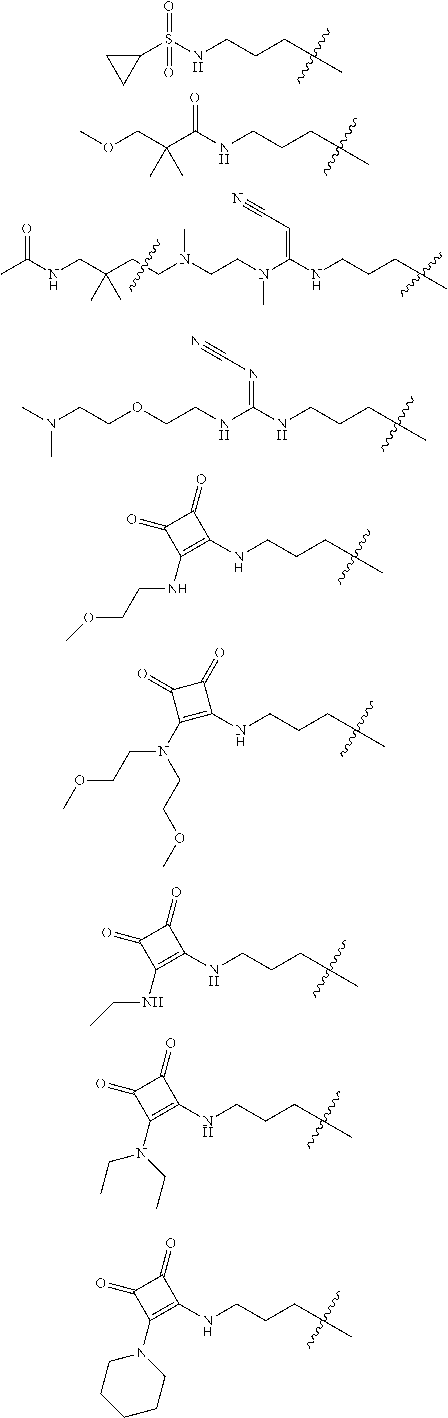

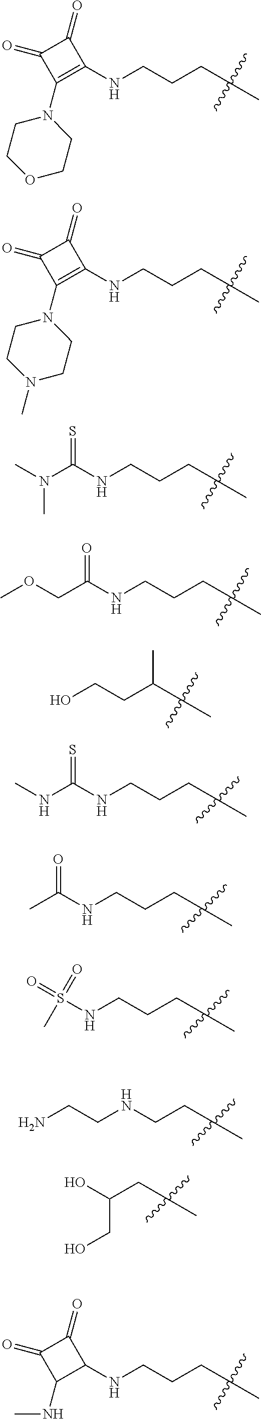

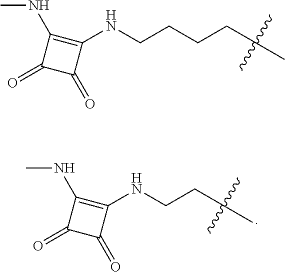 [SMILES: *CCCCNc1c(NC)c(=O)c1=O ; *CCNc1c(NC)c(=O)c1=O]

In some embodiments,

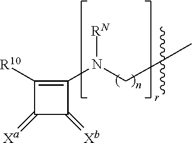

is selected from any of the following groups:

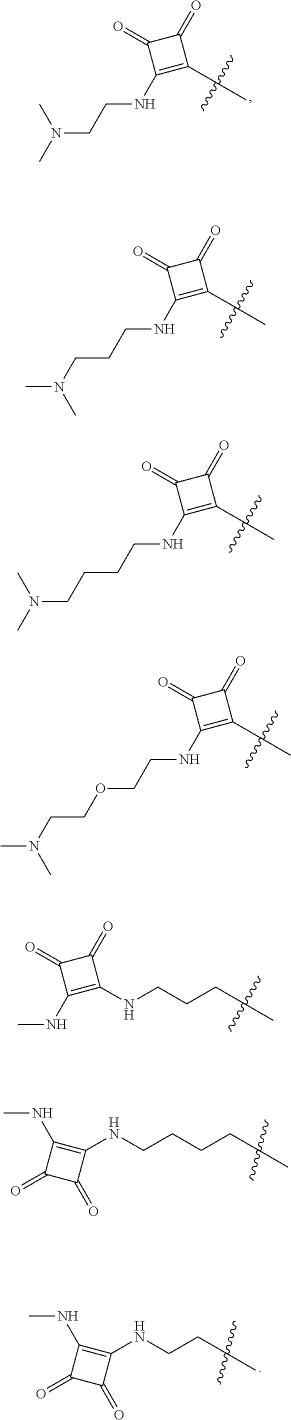

In some embodiments, a compound of Formula (III) further comprises an anion. As described herein, and anion can be any anion capable of reacting with an amine to form an ammonium salt. Examples include, but are not limited to, chloride, bromide, iodide, fluoride, acetate, formate, trifluoroacetate, difluoroacetate, trichloroacetate, and phosphate.

In some embodiments the compound of any of the formulae described herein is suitable for making a nanoparticle composition for intramuscular administration.

In some embodiments, R² and R³, together with the atom to which they are attached, form a heterocycle or carbocycle. In some embodiments, R² and R³, together with the atom to which they are attached, form a 5- to 14-membered aromatic or non-aromatic heterocycle having one or more heteroatoms selected from N, O, S, and P. In some embodiments, R² and R³, together with the atom to which they are attached, form an optionally substituted C₃₋₂₀ carbocycle (e.g., C₃₋₁₈ carbocycle, C₃₋₁₅ carbocycle, C₃₋₁₂ carbocycle, or C₃₋₁₀ carbocycle), either aromatic or non-aromatic. In some embodiments, R² and R³, together with the atom to which they are attached, form a C₃₋₆ carbocycle. In other embodiments, R² and R³, together with the atom to which they are attached, form a C₆ carbocycle, such as a cyclohexyl or phenyl group. In certain embodiments, the heterocycle or C₃₋₆ carbocycle is substituted with one or more alkyl groups (e.g., at the same ring atom or at adjacent or non-adjacent ring atoms). For example, R² and R³, together with the atom to which they are attached, may form a cyclohexyl or phenyl group bearing one or more C₅ alkyl substitutions. In certain embodiments, the heterocycle or C₃₋₆ carbocycle formed by R² and R³, is substituted with a carbocycle groups. For example, R² and R³, together with the atom to which they are attached, may form a cyclohexyl or phenyl group that is substituted with cyclohexyl. In some embodiments, R² and R³, together with the atom to which they are attached, form a C₇₋₁₅ carbocycle, such as a cycloheptyl, cyclopentadecanyl, or naphthyl group.

In some embodiments, R⁴ is selected from —(CH₂)ₙQ and —(CH₂)ₙCHQR. In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(R)S(O)₂R⁸, —N(H)C(O)N(H)(R), —N(R)C(S)N(R)₂, —N(H)C(S)N(R)₂, —N(H)C(S)N(H)(R), and a heterocycle. In other embodiments, Q is selected from the group consisting of an imidazole, a pyrimidine, and a purine.

In some embodiments, R² and R³, together with the atom to which they are attached, form a heterocycle or carbocycle. In some embodiments, R² and R³, together with the atom to which they are attached, form a C₃₋₆ carbocycle. In some embodiments, R² and R³, together with the atom to which they are attached, form a C₆ carbocycle. In some embodiments, R² and R³, together with the atom to which they are attached, form a phenyl group. In some embodiments, R² and R³, together with the atom to which they are attached, form a cyclohexyl group. In some embodiments, R² and R³, together with the atom to which they are attached, form a heterocycle. In certain embodiments, the heterocycle or C₃₋₆ carbocycle is substituted with one or more alkyl groups (e.g., at the same ring atom or at adjacent or non-adjacent ring atoms). For example, R² and R³, together with the atom to which they are attached, may form a phenyl group bearing one or more C₅ alkyl substitutions.

In some embodiments, at least one occurrence of R⁵ and R⁶ is C₁₋₃ alkyl, e.g., methyl. In some embodiments, one of the R⁵ and R⁶ adjacent to M is C₁₋₃ alkyl, e.g., methyl, and the other is H. In some embodiments, one of the R⁵ and R⁶ adjacent to M is C₁₋₃ alkyl, e.g., methyl and the other is H, and M is —OC(O)— or —C(O)O—.

In some embodiments, at most one occurrence of R⁵ and R⁶ is C₁₋₃ alkyl, e.g., methyl. In some embodiments, one of the R⁵ and R⁶ adjacent to M is C₁₋₃ alkyl, e.g., methyl, and the other is H. In some embodiments, one of the R⁵ and R⁶ adjacent to M is C₁₋₃ alkyl, e.g., methyl and the other is H, and M is —OC(O)— or —C(O)O—.

In some embodiments, at least one occurrence of R⁵ and R⁶ is methyl.

The compounds of any one of formula (VI), (VI-a), (VII), (VIIa), (VIIb), (VIIc), (VIId), (VIII), (VIIIa), (VIIIb), (VIIIc) or (VIIId) include one or more of the following features when applicable.

In some embodiments, r is 0. In some embodiments, r is 1.

In some embodiments, n is 2, 3, or 4. In some embodiments, n is 2. In some embodiments, n is 4. In some embodiments, n is not 3.

In some embodiments, R^{N} is H. In some embodiments, R^{N} is C₁₋₃ alkyl. For example, in some embodiments R^{N} is C₁ alkyl. For example, in some embodiments R^{N} is C₂ alkyl. For example, in some embodiments R^{N} is C₂ alkyl.

In some embodiments, X^{a} is O. In some embodiments, X^{a} is S. In some embodiments, X^{b} is O. In some embodiments, X^{b} is S.

In some embodiments, R¹⁰ is selected from the group consisting of N(R)₂, —NH(CH₂)ₜ₁N(R)₂, —NH(CH₂)ₚ₁(CH₂)_{q1}N(R)₂, —NH(CH₂)siOR, —N((CH₂)ₛ₁OR)₂, and a heterocycle.

In some embodiments, R¹⁰ is selected from the group consisting of —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, and a heterocycle.

In some embodiments wherein R¹⁰ is —NH(CH₂)ₒN(R)₂, o is 2, 3, or 4.

In some embodiments wherein —NH(CH₂)ₚ₁O(CH₂)_{q1}N(R)₂, p¹ is 2. In some embodiments wherein —NH(CH₂)ₚ₁(CH₂)_{q1}N(R)₂, q¹ is 2.

In some embodiments wherein R¹⁰ is —N((CH₂)ₛ₁OR)₂, s¹ is 2.

In some embodiments wherein R¹⁰ is —NH(CH₂)ₒN(R)₂, —NH(CH₂)ₚ(CH₂)O(CH₂)_{q}N(R)₂, —NH(CH₂)ₛOR, or —N((CH₂)ₛOR)₂, R is H or C₁-C₃ alkyl. For example, in some embodiments, R is C₁ alkyl. For example, in some embodiments, R is C₂ alkyl. For example, in some embodiments, R is H. For example, in some embodiments, R is H and one R is C₁-C₃ alkyl. For example, in some embodiments, R is H and one R is C₁ alkyl. For example, in some embodiments, R is H and one R is C₂ alkyl. In some embodiments wherein R¹⁰ is —NH(CH₂)ₜ₁N(R)₂, —NH(CH₂)ₚ₁O(CH₂)_{q1}N(R)₂, —NH(CH₂)ₛ₁OR, or —N((CH₂)ₛ₁OR)₂, each R is C₂-C₄ alkyl.

For example, in some embodiments, one R is H and one R is C₂-C₄ alkyl. In some embodiments, R¹⁰ is a heterocycle. For example, in some embodiments, R¹⁰ is morpholinyl. For example, in some embodiments, R¹⁰ is methyhlpiperazinyl.

In some embodiments, each occurrence of R⁵ and R⁶ is H.

In some embodiments, the compound of Formula (I) is selected from the group consisting of:

| Cpd | Structure |
|---|---|
| I 1 | 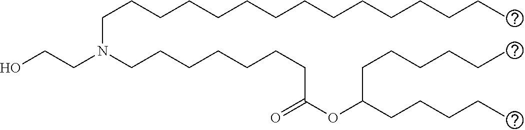 [SMILES: CCCCCCCCCCCCCCCN(CCO)CCCCCCCC(=O)OC(CCCCC)CCCCCC] |
| I 2 | 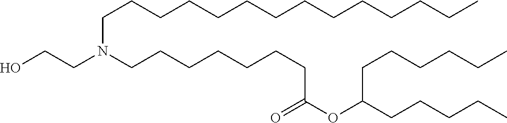 [SMILES: CCCCCCCCCCCCCCN(CCO)CCCCCCCC(=O)OC(CCCCC)CCCCCC] |
| I 3 | 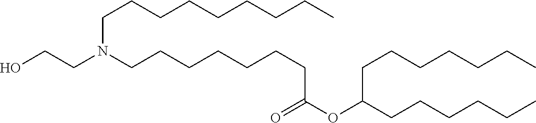 [SMILES: CCCCCCCCCN(CCO)CCCCCCCC(=O)OC(CCCCCC)CCCCCCC] |
| I 4 | 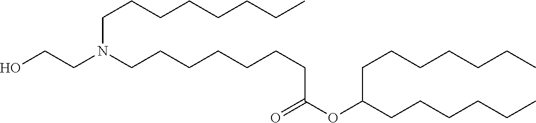 [SMILES: CCCCCCCCN(CCO)CCCCCCCC(=O)OC(CCCCCC)CCCCCCC] |
| I 5 | 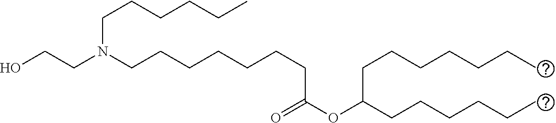 [SMILES: CCCCCCCC(CCCCCC)OC(=O)CCCCCCCN(CCO)CCCCCC] |
| I 6 | 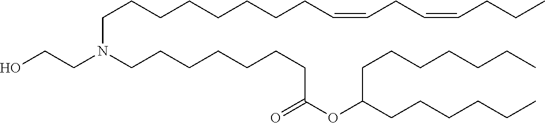 [SMILES: CCC/C=C\C/C=C\CCCCCCCCN(CCO)CCCCCCCC(=O)OC(CCCCCC)CCCCCCC] |
| I 7 | 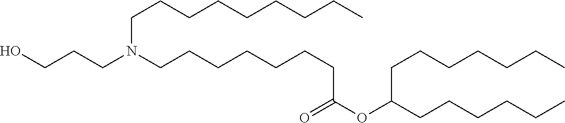 [SMILES: CCCCCCCCCN(CCCO)CCCCCCCC(=O)OC(CCCCCC)CCCCCCC] |
| I 8 | 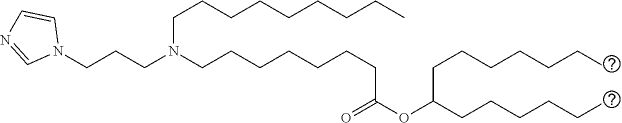 [SMILES: CCCCCCCCCN(CCCCCCCC(=O)OC(CCCCCC)CCCCCCC)CCCn1ccnc1] |
| I 9 | 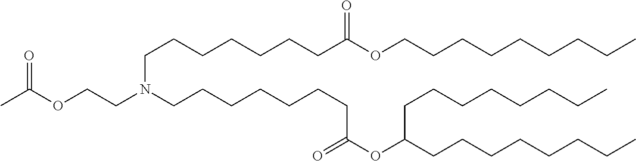 [SMILES: CCCCCCCCCOC(=O)CCCCCCCN(CCCCCCCC(=O)OC(CCCCCCCC)CCCCCCCC)CCOC(C)=O] |
| I 10 | 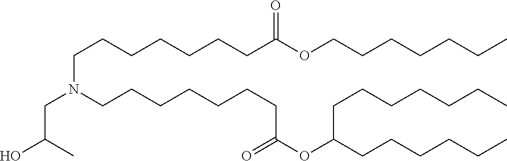 [SMILES: CCCCCCCOC(=O)CCCCCCCN(CCCCCCCC(=O)OC(CCCCCC)CCCCCCC)CC(C)O] |
| I 11 | 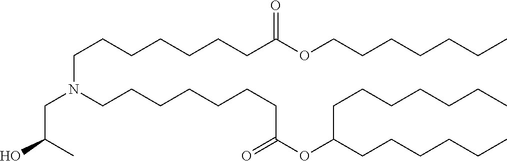 [SMILES: CCCCCCCOC(=O)CCCCCCCN(CCCCCCCC(=O)OC(CCCCCC)CCCCCCC)C[C@@H](C)O] |
| I 12 | 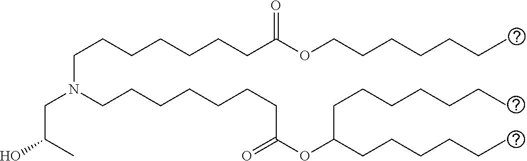 [SMILES: CCCCCCCOC(=O)CCCCCCCN(CCCCCCCC(=O)OC(CCCCCC)CCCCCCC)C[C@H](C)O] |
| I 13 | 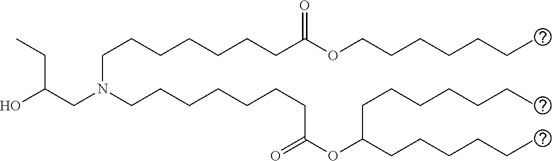 [SMILES: CCCCCCCOC(=O)CCCCCCCN(CCCCCCCC(=O)OC(CCCCCC)CCCCCCC)CC(O)CC] |
| I 14 | 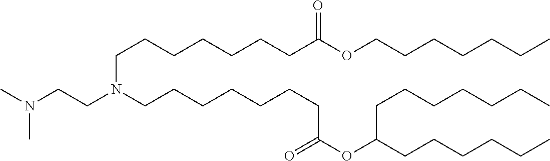 [SMILES: CCCCCCCOC(=O)CCCCCCCN(CCCCCCCC(=O)OC(CCCCCC)CCCCCCC)CCN(C)C] |
| I 15 | 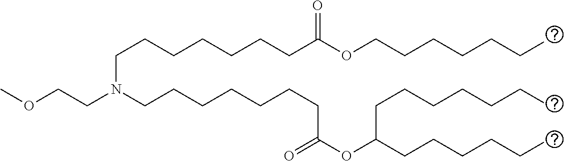 [SMILES: CCCCCCCOC(=O)CCCCCCCN(CCCCCCCC(=O)OC(CCCCCC)CCCCCCC)CCOC] |
| I 16 | 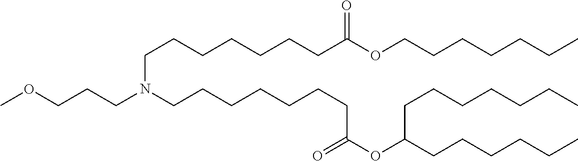 [SMILES: CCCCCCCOC(=O)CCCCCCCN(CCCCCCCC(=O)OC(CCCCCC)CCCCCCC)CCCOC] |
| I 17 | 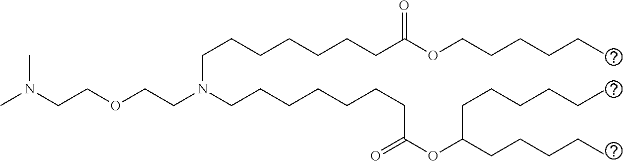 [SMILES: CCCCCCOC(=O)CCCCCCCN(CCCCCCCC(=O)OC(CCCCC)CCCCCC)CCOCCN(C)C] |
| I 18 | 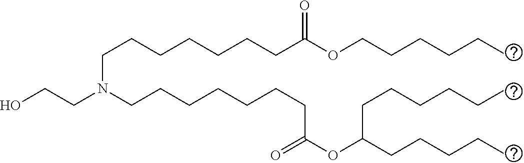 [SMILES: CCCCCCOC(=O)CCCCCCCN(CCO)CCCCCCCC(=O)OC(CCCCC)CCCCCC] |
| I 19 | 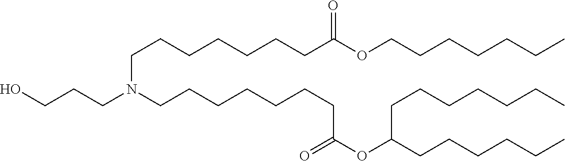 [SMILES: CCCCCCCOC(=O)CCCCCCCN(CCCO)CCCCCCCC(=O)OC(CCCCCC)CCCCCCC] |
| I 20 | 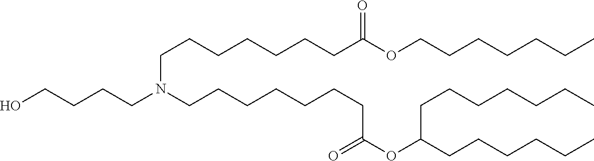 [SMILES: CCCCCCCOC(=O)CCCCCCCN(CCCCO)CCCCCCCC(=O)OC(CCCCCC)CCCCCCC] |
| I 21 | 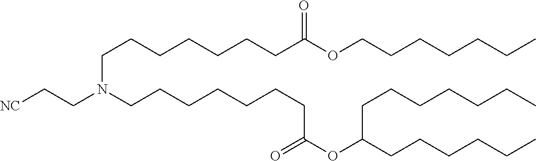 [SMILES: CCCCCCCOC(=O)CCCCCCCN(CCC#N)CCCCCCCC(=O)OC(CCCCCC)CCCCCCC] |
| I 22 | 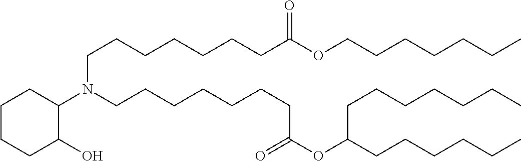 [SMILES: CCCCCCCOC(=O)CCCCCCCN(CCCCCCCC(=O)OC(CCCCCC)CCCCCCC)C1CCCCC1O] |
| I 23 | 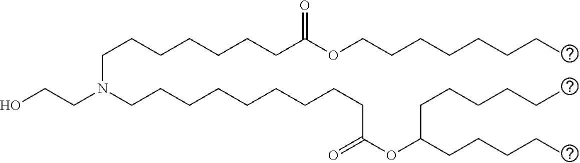 [SMILES: CCCCCCCCOC(=O)CCCCCCCN(CCO)CCCCCCCCCC(=O)OC(CCCCC)CCCCCC] |
| I 24 | 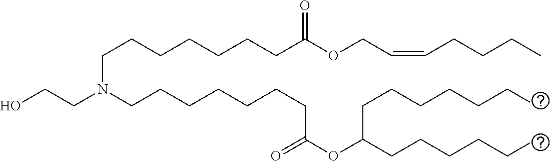 [SMILES: CCCC/C=C\COC(=O)CCCCCCCN(CCO)CCCCCCCC(=O)OC(CCCCCC)CCCCCCC] |
| I 25 | 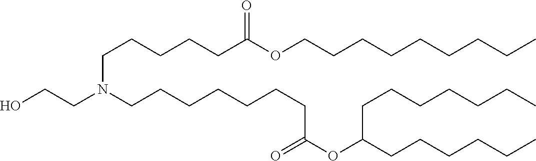 [SMILES: CCCCCCCCCOC(=O)CCCCCN(CCO)CCCCCCCC(=O)OC(CCCCCC)CCCCCCC] |
| I 26 | 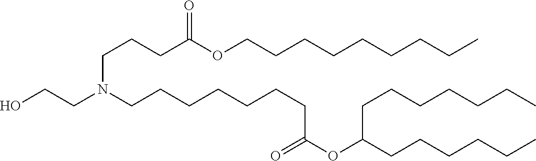 [SMILES: CCCCCCCCCOC(=O)CCCN(CCO)CCCCCCCC(=O)OC(CCCCCC)CCCCCCC] |
| I 27 | 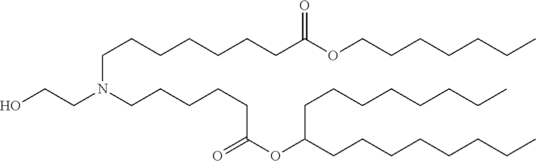 [SMILES: CCCCCCCCC(CCCCCCCC)OC(=O)CCCCCN(CCO)CCCCCCCC(=O)OCCCCCCC] |
| I 28 | 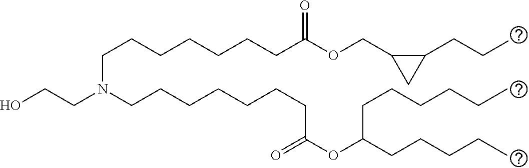 [SMILES: CCCCCCC(CCCCC)OC(=O)CCCCCCCN(CCO)CCCCCCCC(=O)OCC1CC1CCC] |
| I 29 | 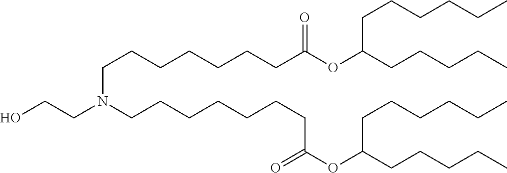 [SMILES: CCCCCCC(CCCCC)OC(=O)CCCCCCCN(CCO)CCCCCCCC(=O)OC(CCCCC)CCCCCC] |
| I 30 | 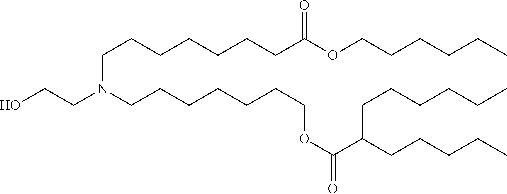 [SMILES: CCCCCCOC(=O)CCCCCCCN(CCO)CCCCCCCOC(=O)C(CCCCC)CCCCCC] |
| I 31 | 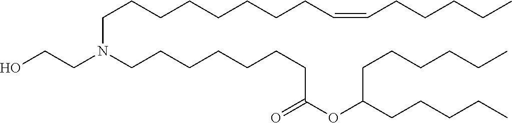 [SMILES: CCCCC/C=C\CCCCCCCCN(CCO)CCCCCCCC(=O)OC(CCCCC)CCCCCC] |
| I 32 | 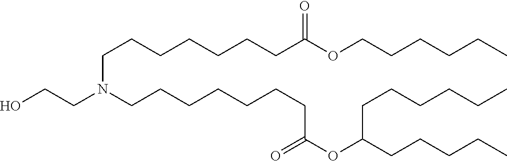 [SMILES: CCCCCCOC(=O)CCCCCCCN(CCO)CCCCCCCC(=O)OC(CCCCC)CCCCCC] |
| I 33 | 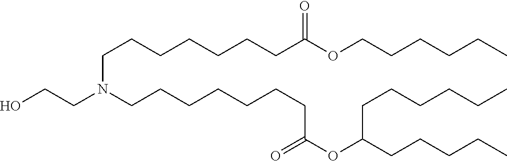 [SMILES: CCCCCCOC(=O)CCCCCCCN(CCO)CCCCCCCC(=O)OC(CCCCC)CCCCCC] |
| I 34 | 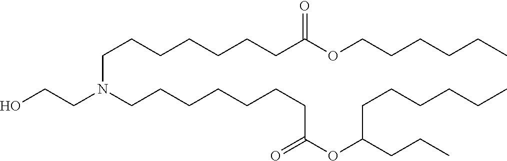 [SMILES: CCCCCCOC(=O)CCCCCCCN(CCO)CCCCCCCC(=O)OC(CCC)CCCCCC] |
| I 35 | 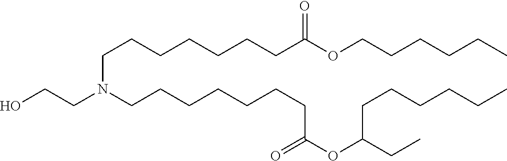 [SMILES: CCCCCCOC(=O)CCCCCCCN(CCO)CCCCCCCC(=O)OC(CC)CCCCCC] |
| I 36 | 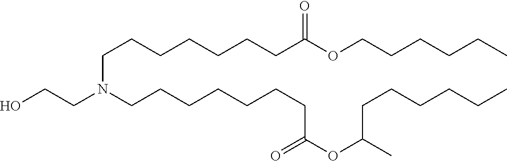 [SMILES: CCCCCCOC(=O)CCCCCCCN(CCO)CCCCCCCC(=O)OC(C)CCCCCC] |
| I 37 | 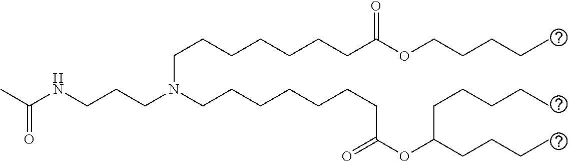 [SMILES: CCCCCOC(=O)CCCCCCCN(CCCCCCCC(=O)OC(CCCC)CCCCC)CCCNC(C)=O] |
| I 38 | 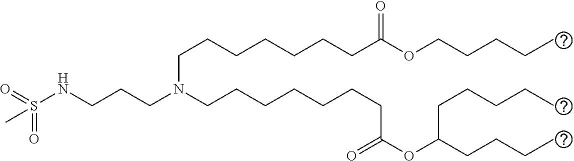 [SMILES: CCCCCOC(=O)CCCCCCCN(CCCCCCCC(=O)OC(CCCC)CCCCC)CCCNS(C)(=O)=O] |
| I 39 | 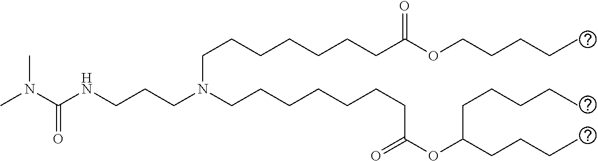 [SMILES: CCCCCOC(=O)CCCCCCCN(CCCCCCCC(=O)OC(CCCC)CCCCC)CCCNC(=O)N(C)C] |
| I 40 | 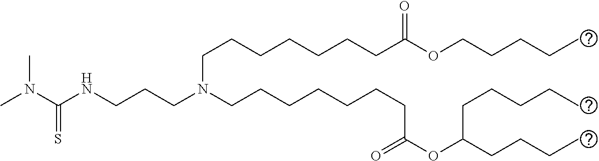 [SMILES: CCCCCOC(=O)CCCCCCCN(CCCCCCCC(=O)OC(CCCC)CCCCC)CCCNC(=S)N(C)C] |
| I 41 | 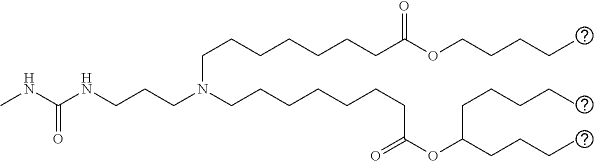 [SMILES: CCCCCOC(=O)CCCCCCCN(CCCCCCCC(=O)OC(CCCC)CCCCC)CCCNC(=O)NC] |
| I 42 | 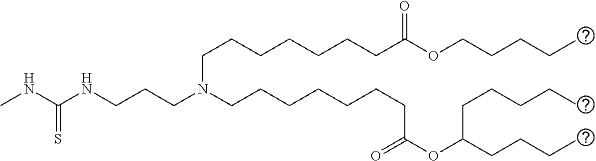 [SMILES: CCCCCOC(=O)CCCCCCCN(CCCCCCCC(=O)OC(CCCC)CCCCC)CCCNC(=S)NC] |
| I 43 | 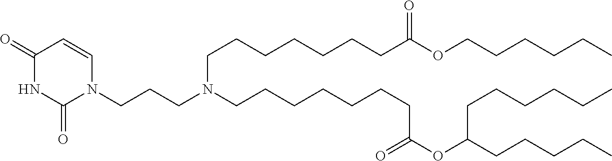 [SMILES: CCCCCCOC(=O)CCCCCCCN(CCCCCCCC(=O)OC(CCCCC)CCCCCC)CCCn1ccc(=O)[nH]c1=O] |
| I 44 | 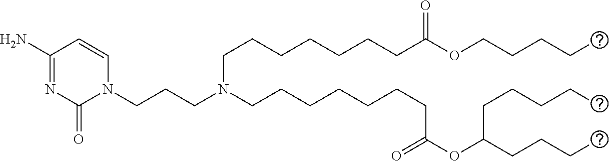 [SMILES: CCCCCOC(=O)CCCCCCCN(CCCCCCCC(=O)OC(CCCC)CCCCC)CCCn1ccc(N)nc1=O] |
| I 45 | 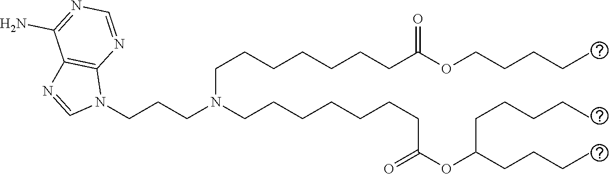 [SMILES: CCCCCOC(=O)CCCCCCCN(CCCCCCCC(=O)OC(CCCC)CCCCC)CCCn1cnc2c(N)ncnc21] |
| I 46 | 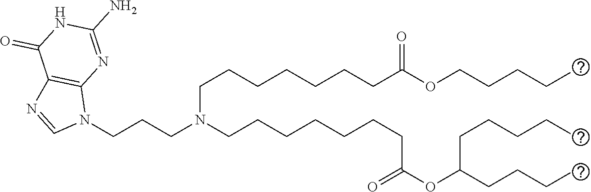 [SMILES: CCCCCOC(=O)CCCCCCCN(CCCCCCCC(=O)OC(CCCC)CCCCC)CCCn1cnc2c(=O)[nH]c(N)nc21] |
| I 47 | 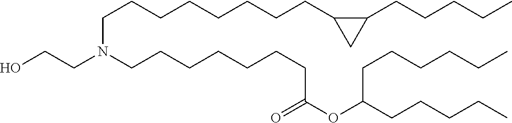 [SMILES: CCCCCCC(CCCCC)OC(=O)CCCCCCCN(CCO)CCCCCCCCC1CC1CCCCC] |
| I 48 | 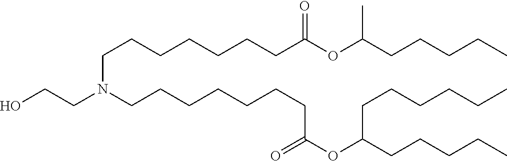 [SMILES: CCCCCCC(CCCCC)OC(=O)CCCCCCCN(CCO)CCCCCCCC(=O)OC(C)CCCCC] |
| I 49 | 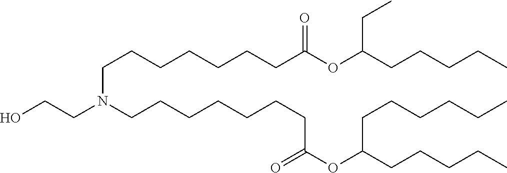 [SMILES: CCCCCCC(CCCCC)OC(=O)CCCCCCCN(CCO)CCCCCCCC(=O)OC(CC)CCCCC] |
| I 50 | 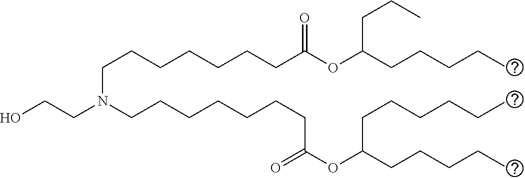 [SMILES: CCCCCCC(CCCCC)OC(=O)CCCCCCCN(CCO)CCCCCCCC(=O)OC(CCC)CCCCC] |
| I 51 | 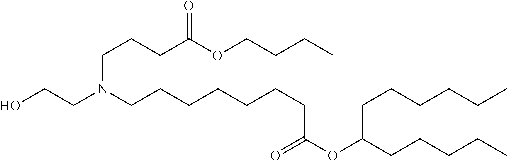 [SMILES: CCCCCCC(CCCCC)OC(=O)CCCCCCCN(CCO)CCCC(=O)OCCCC] |
| I 52 | 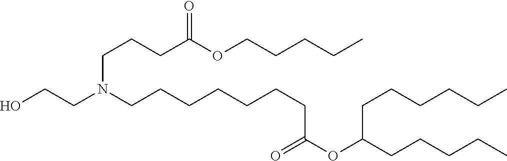 [SMILES: CCCCCCC(CCCCC)OC(=O)CCCCCCCN(CCO)CCCC(=O)OCCCCC] |
| I 53 | 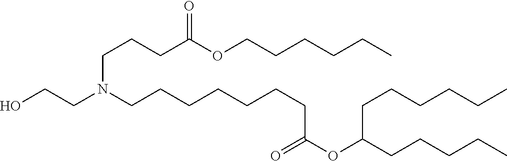 [SMILES: CCCCCCOC(=O)CCCN(CCO)CCCCCCCC(=O)OC(CCCCC)CCCCCC] |
| I 54 | 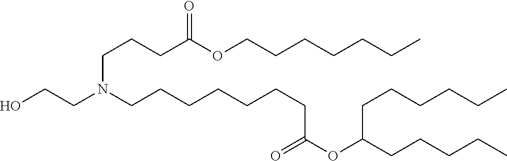 [SMILES: CCCCCCCOC(=O)CCCN(CCO)CCCCCCCC(=O)OC(CCCCC)CCCCCC] |
| I 55 | 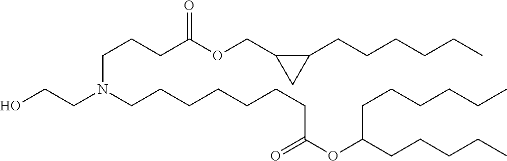 [SMILES: CCCCCCC(CCCCC)OC(=O)CCCCCCCN(CCO)CCCC(=O)OCC1CC1CCCCCC] |
| I 56 | 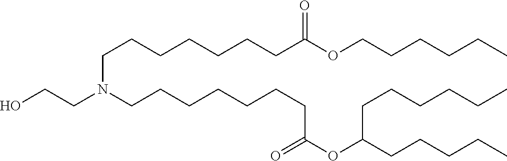 [SMILES: CCCCCCOC(=O)CCCCCCCN(CCO)CCCCCCCC(=O)OC(CCCCC)CCCCCC] |
| I 57 | 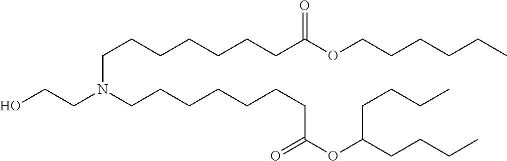 [SMILES: CCCCCCOC(=O)CCCCCCCN(CCO)CCCCCCCC(=O)OC(CCCC)CCCC] |
| I 58 | 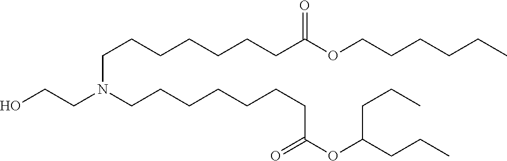 [SMILES: CCCCCCOC(=O)CCCCCCCN(CCO)CCCCCCCC(=O)OC(CCC)CCC] |
| I 59 | 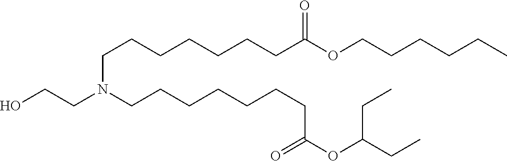 [SMILES: CCCCCCOC(=O)CCCCCCCN(CCO)CCCCCCCC(=O)OC(CC)CC] |
| I 60 | 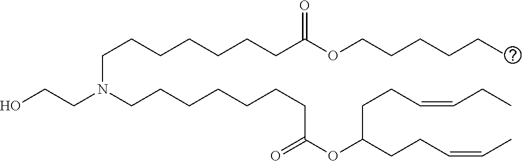 [SMILES: C/C=C\CCC(CC/C=C\CC)OC(=O)CCCCCCCN(CCO)CCCCCCCC(=O)OCCCCCC] |
| I 61 | 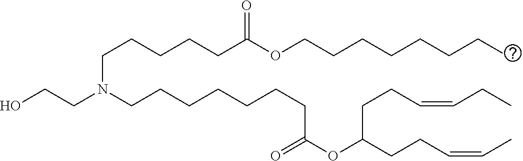 [SMILES: C/C=C\CCC(CC/C=C\CC)OC(=O)CCCCCCCN(CCO)CCCCCC(=O)OCCCCCCCC] |

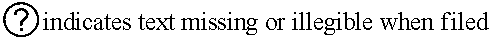

In further embodiments, the compound of Formula (I I) is selected from the group consisting of:

| Cpd | Structure |
|---|---|
| I 62 | 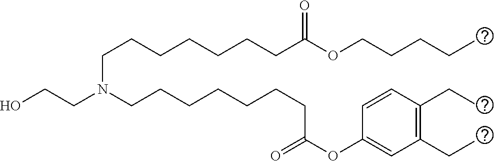 [SMILES: CCCCCOC(=O)CCCCCCCN(CCO)CCCCCCCC(=O)Oc1ccc(CC)c(CC)c1] |
| I 63 | 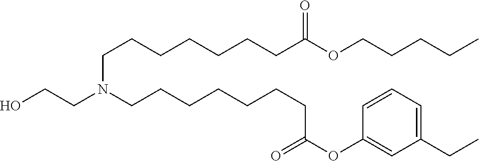 [SMILES: CCCCCOC(=O)CCCCCCCN(CCO)CCCCCCCC(=O)Oc1cccc(CC)c1] |
| I 64 | 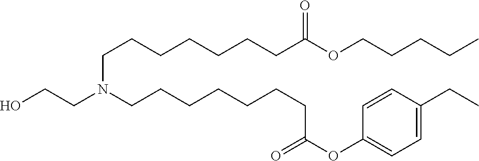 [SMILES: CCCCCOC(=O)CCCCCCCN(CCO)CCCCCCCC(=O)Oc1ccc(CC)cc1] |

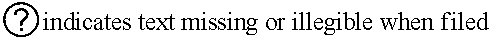

In some embodiments, the compound of Formula (I I) or Formula (I IV) is selected from the group consisting of:

| Cpd | Structure |
|---|---|
| I 65 | 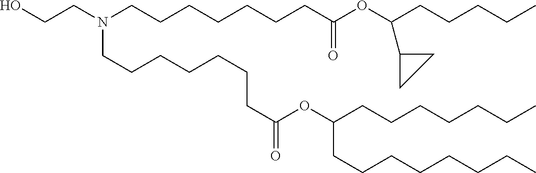 [SMILES: CCCCCCCCC(CCCCCCC)OC(=O)CCCCCCCN(CCO)CCCCCCCC(=O)OC(CCCCC)C1CC1] |
| I 66 | 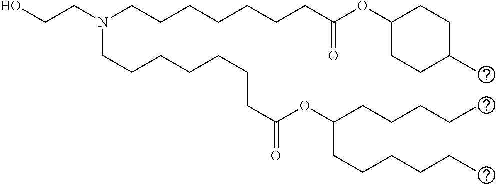 [SMILES: CCCCCCC(CCCCC)OC(=O)CCCCCCCN(CCO)CCCCCCCC(=O)OC1CCC(C)CC1] |
| I 67 | 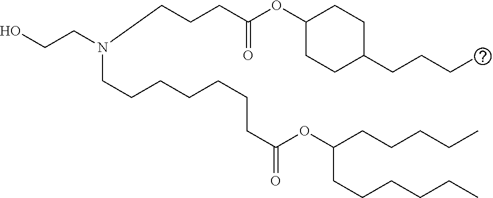 [SMILES: CCCCCCC(CCCCC)OC(=O)CCCCCCCN(CCO)CCCC(=O)OC1CCC(CCCC)CC1] |
| I 68 | 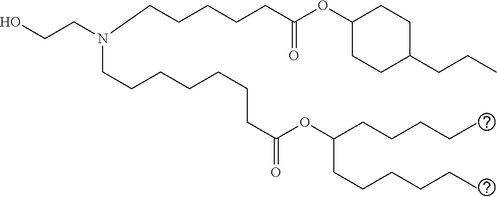 [SMILES: CCCCCCC(CCCCC)OC(=O)CCCCCCCN(CCO)CCCCCC(=O)OC1CCC(CCC)CC1] |
| I 69 | 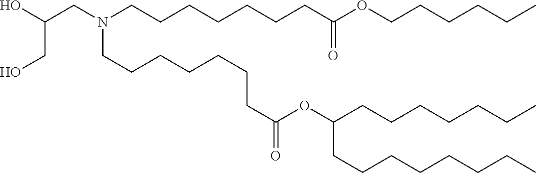 [SMILES: CCCCCCCCC(CCCCCCC)OC(=O)CCCCCCCN(CCCCCCCC(=O)OCCCCCC)CC(O)CO] |
| I 70 | 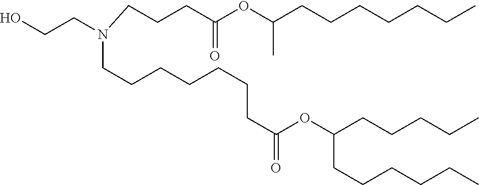 [SMILES: CCCCCCCC(C)OC(=O)CCCN(CCO)CCCCCCCC(=O)OC(CCCCC)CCCCCC] |
| I 71 | 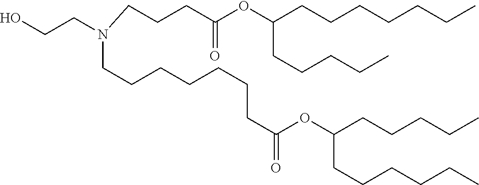 [SMILES: CCCCCCCC(CCCCC)OC(=O)CCCN(CCO)CCCCCCCC(=O)OC(CCCCC)CCCCCC] |
| I 72 | 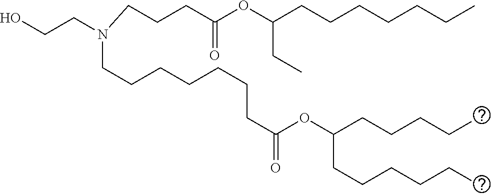 [SMILES: CCCCCCCC(CC)OC(=O)CCCN(CCO)CCCCCCCC(=O)OC(CCCCC)CCCCCC] |
| I 73 | 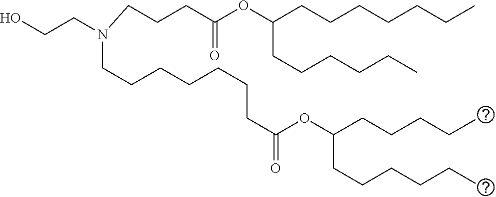 [SMILES: CCCCCCCC(CCCCCC)OC(=O)CCCN(CCO)CCCCCCCC(=O)OC(CCCCC)CCCCCC] |
| I 74 | 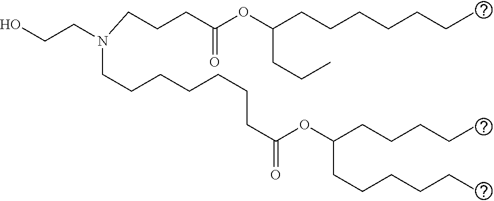 [SMILES: CCCCCCCC(CCC)OC(=O)CCCN(CCO)CCCCCCCC(=O)OC(CCCCC)CCCCCC] |
| I 75 | 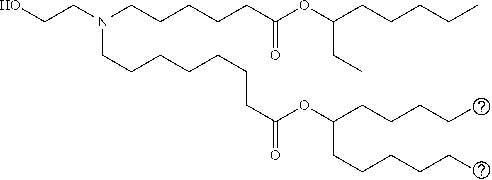 [SMILES: CCCCCCC(CCCCC)OC(=O)CCCCCCCN(CCO)CCCCCC(=O)OC(CC)CCCCC] |
| I 76 | 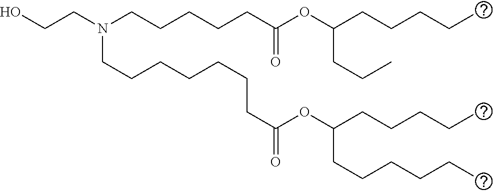 [SMILES: CCCCCCC(CCCCC)OC(=O)CCCCCCCN(CCO)CCCCCC(=O)OC(CCC)CCCCC] |
| I 77 | 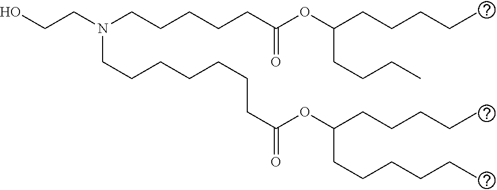 [SMILES: CCCCCCC(CCCCC)OC(=O)CCCCCCCN(CCO)CCCCCC(=O)OC(CCCC)CCCCC] |
| I 78 | 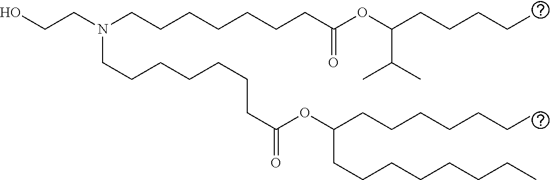 [SMILES: CCCCCCCCC(CCCCCCC)OC(=O)CCCCCCCN(CCO)CCCCCCCC(=O)OC(CCCCC)C(C)C] |
| I 79 | 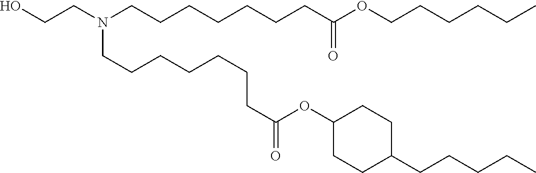 [SMILES: CCCCCCOC(=O)CCCCCCCN(CCO)CCCCCCCC(=O)OC1CCC(CCCCC)CC1] |
| I 80 | 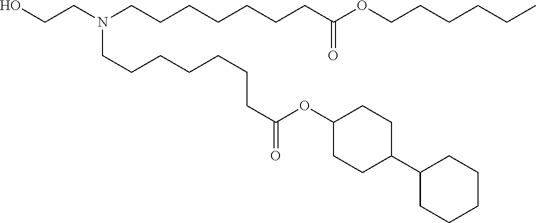 [SMILES: CCCCCCOC(=O)CCCCCCCN(CCO)CCCCCCCC(=O)OC1CCC(C2CCCCC2)CC1] |
| I 81 | 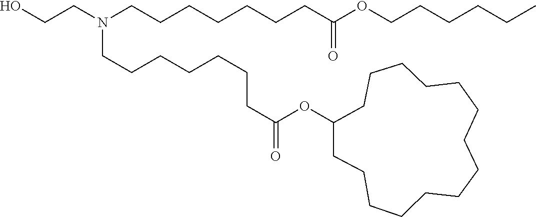 [SMILES: CCCCCCOC(=O)CCCCCCCN(CCO)CCCCCCCC(=O)OC1CCCCCCCCCCCCCC1] |
| I 82 | 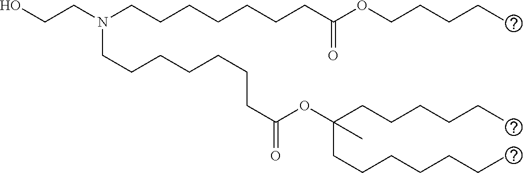 [SMILES: CCCCCCCC(C)(CCCCCC)OC(=O)CCCCCCCN(CCO)CCCCCCCC(=O)OCCCCC] |
| I 83 | 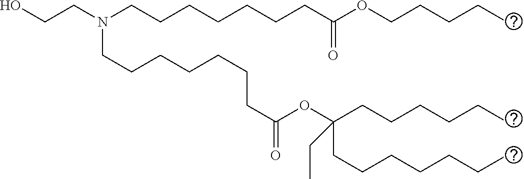 [SMILES: CCCCCCCC(CC)(CCCCCC)OC(=O)CCCCCCCN(CCO)CCCCCCCC(=O)OCCCCC] |
| I 84 | 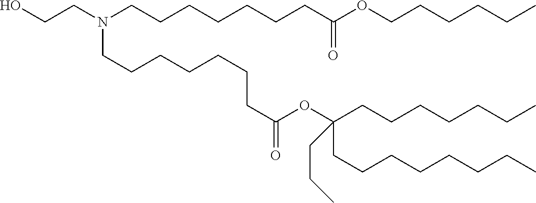 [SMILES: CCCCCCCCC(CCC)(CCCCCCC)OC(=O)CCCCCCCN(CCO)CCCCCCCC(=O)OCCCCCC] |
| I 85 | 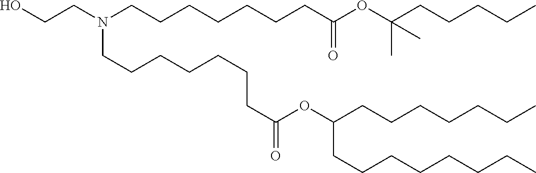 [SMILES: CCCCCCCCC(CCCCCCC)OC(=O)CCCCCCCN(CCO)CCCCCCCC(=O)OC(C)(C)CCCCC] |
| I 86 | 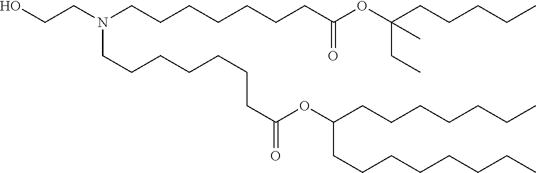 [SMILES: CCCCCCCCC(CCCCCCC)OC(=O)CCCCCCCN(CCO)CCCCCCCC(=O)OC(C)(CC)CCCCC] |
| I 87 | 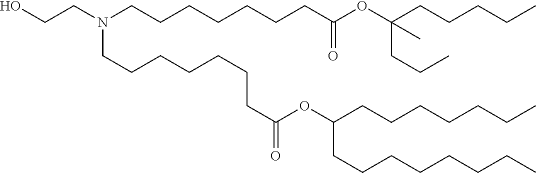 [SMILES: CCCCCCCCC(CCCCCCC)OC(=O)CCCCCCCN(CCO)CCCCCCCC(=O)OC(C)(CCC)CCCCC] |
| I 88 | 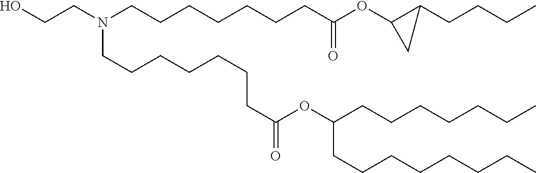 [SMILES: CCCCCCCCC(CCCCCCC)OC(=O)CCCCCCCN(CCO)CCCCCCCC(=O)OC1CC1CCCC] |
| I 89 | 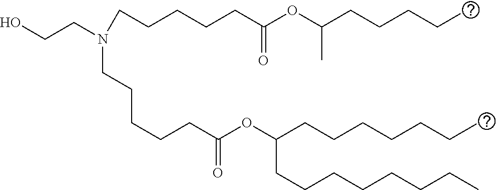 [SMILES: CCCCCCCCC(CCCCCCC)OC(=O)CCCCCN(CCO)CCCCCC(=O)OC(C)CCCCC] |
| I 90 | 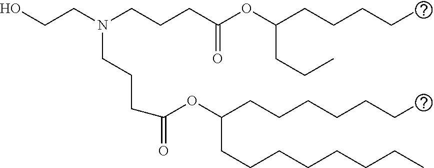 [SMILES: CCCCCCCCC(CCCCCCC)OC(=O)CCCN(CCO)CCCC(=O)OC(CCC)CCCCC] |
| I 91 | 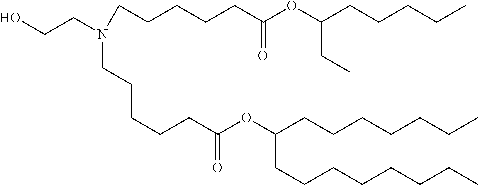 [SMILES: CCCCCCCCC(CCCCCCC)OC(=O)CCCCCN(CCO)CCCCCC(=O)OC(CC)CCCCC] |
| I 92 | 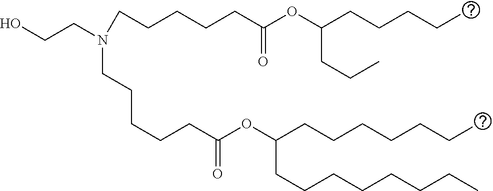 [SMILES: CCCCCCCCC(CCCCCCC)OC(=O)CCCCCN(CCO)CCCCCC(=O)OC(CCC)CCCCC] |
| I 93 | 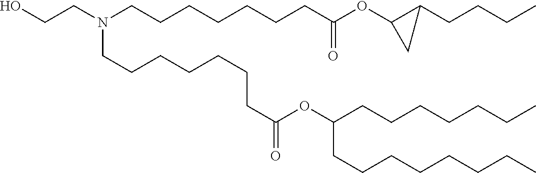 [SMILES: CCCCCCCCC(CCCCCCC)OC(=O)CCCCCCCN(CCO)CCCCCCCC(=O)OC1CC1CCCC] |
| I 94 | 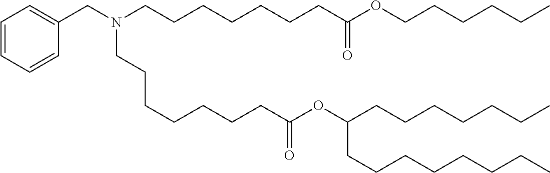 [SMILES: CCCCCCCCC(CCCCCCC)OC(=O)CCCCCCCN(CCCCCCCC(=O)OCCCCCC)Cc1ccccc1] |
| I 95 | 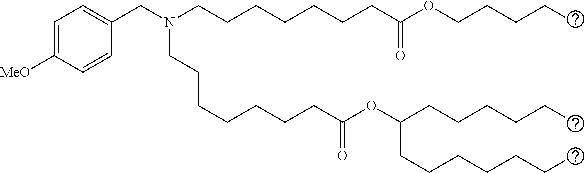 [SMILES: CCCCCCCC(CCCCCC)OC(=O)CCCCCCCN(CCCCCCCC(=O)OCCCCC)Cc1ccc(OC)cc1] |
| I 96 | 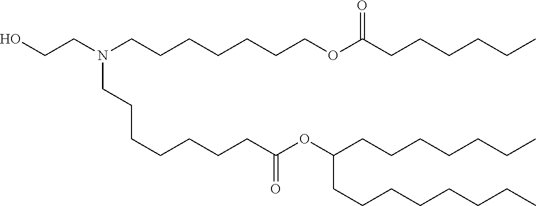 [SMILES: CCCCCCCCC(CCCCCCC)OC(=O)CCCCCCCN(CCO)CCCCCCCOC(=O)CCCCCC] |
| I 97 | 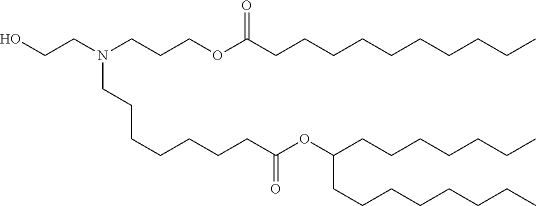 [SMILES: CCCCCCCCCCC(=O)OCCCN(CCO)CCCCCCCC(=O)OC(CCCCCCC)CCCCCCCC] |
| I 98 | 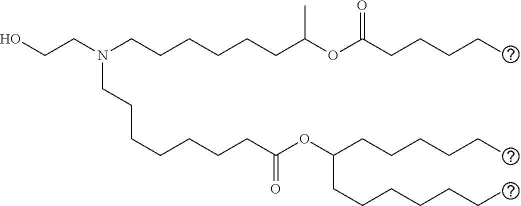 [SMILES: CCCCCCCC(CCCCCC)OC(=O)CCCCCCCN(CCO)CCCCCCC(C)OC(=O)CCCCC] |
| I 99 | 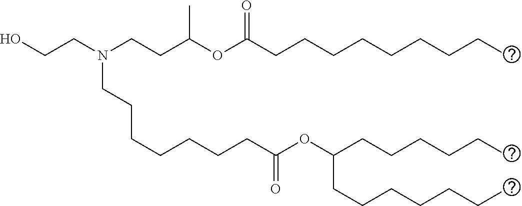 [SMILES: CCCCCCCCCC(=O)OC(C)CCN(CCO)CCCCCCCC(=O)OC(CCCCCC)CCCCCCC] |
| I 100 | 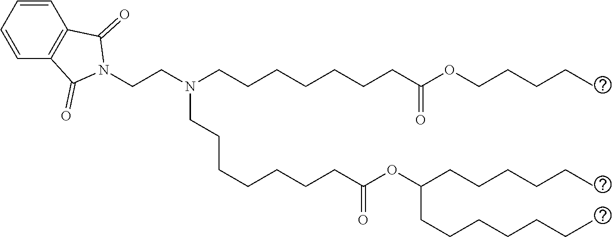 [SMILES: CCCCCCCC(CCCCCC)OC(=O)CCCCCCCN(CCCCCCCC(=O)OCCCCC)CCN1C(=O)c2ccccc2C1=O] |
| I 101 | 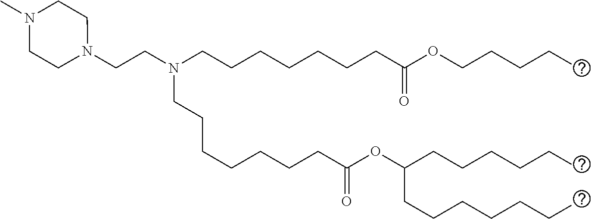 [SMILES: CCCCCCCC(CCCCCC)OC(=O)CCCCCCCN(CCCCCCCC(=O)OCCCCC)CCN1CCN(C)CC1] |
| I 102 | 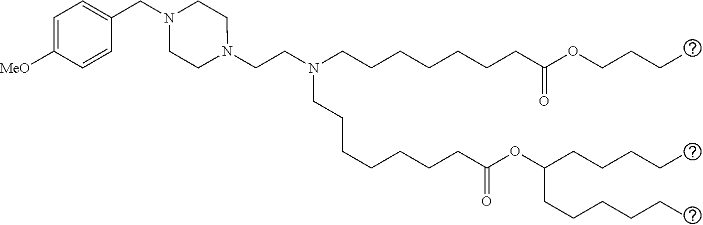 [SMILES: CCCCCCC(CCCCC)OC(=O)CCCCCCCN(CCCCCCCC(=O)OCCCC)CCN1CCN(Cc2ccc(OC)cc2)CC1] |
| I 103 | 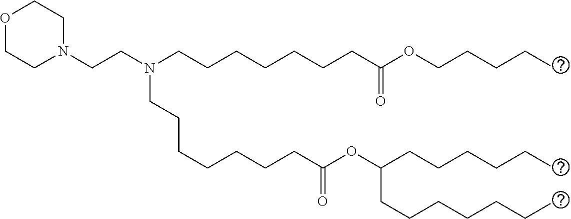 [SMILES: CCCCCCCC(CCCCCC)OC(=O)CCCCCCCN(CCCCCCCC(=O)OCCCCC)CCN1CCOCC1] |
| I 104 | 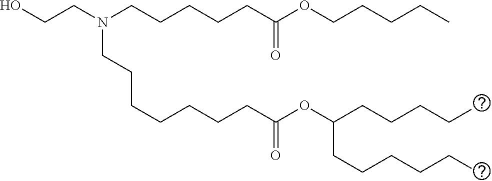 [SMILES: CCCCCCC(CCCCC)OC(=O)CCCCCCCN(CCO)CCCCCC(=O)OCCCCC] |
| I 105 | 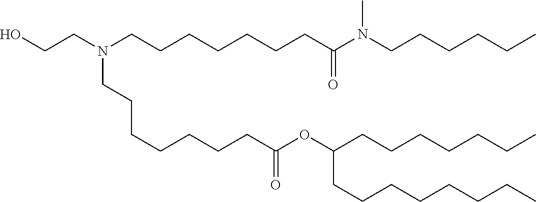 [SMILES: CCCCCCCCC(CCCCCCC)OC(=O)CCCCCCCN(CCO)CCCCCCCC(=O)N(C)CCCCCC] |
| I 106 | 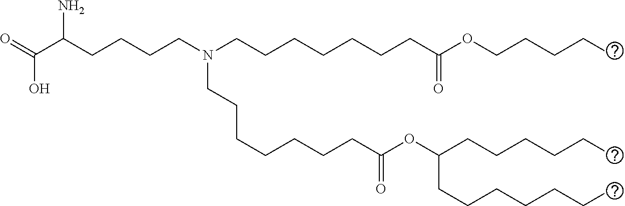 [SMILES: CCCCCCCC(CCCCCC)OC(=O)CCCCCCCN(CCCCCCCC(=O)OCCCCC)CCCCC(N)C(=O)O] |
| I 107 | 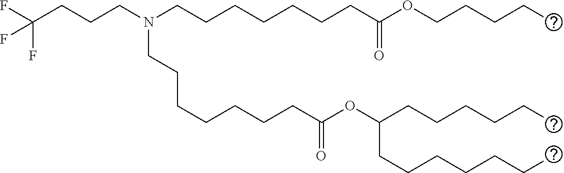 [SMILES: CCCCCCCC(CCCCCC)OC(=O)CCCCCCCN(CCCCCCCC(=O)OCCCCC)CCCC(F)(F)F] |
| I 108 | 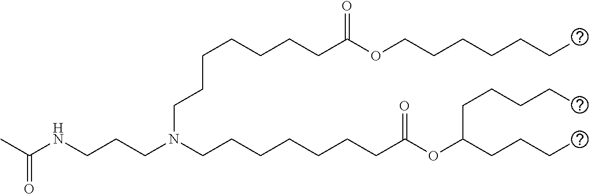 [SMILES: CCCCCCCOC(=O)CCCCCCCN(CCCCCCCC(=O)OC(CCCC)CCCCC)CCCNC(C)=O] |
| I 109 | 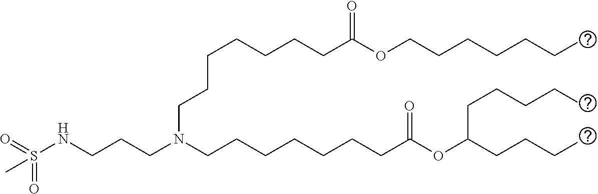 [SMILES: CCCCCCCOC(=O)CCCCCCCN(CCCCCCCC(=O)OC(CCCC)CCCCC)CCCNS(C)(=O)=O] |
| I 110 | 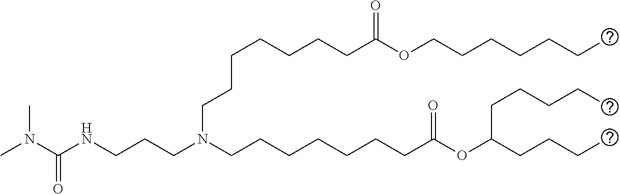 [SMILES: CCCCCCCOC(=O)CCCCCCCN(CCCCCCCC(=O)OC(CCCC)CCCCC)CCCNC(=O)N(C)C] |
| I 111 | 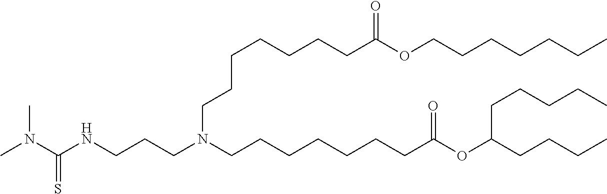 [SMILES: CCCCCCCOC(=O)CCCCCCCN(CCCCCCCC(=O)OC(CCCC)CCCCC)CCCNC(=S)N(C)C] |
| I 112 | 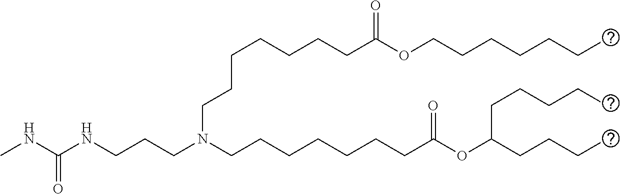 [SMILES: CCCCCCCOC(=O)CCCCCCCN(CCCCCCCC(=O)OC(CCCC)CCCCC)CCCNC(=O)NC] |
| I 113 | 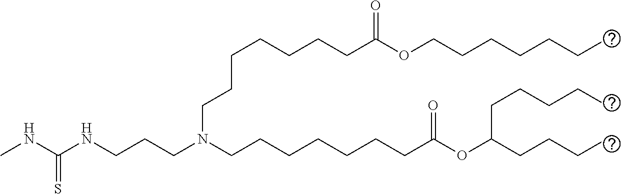 [SMILES: CCCCCCCOC(=O)CCCCCCCN(CCCCCCCC(=O)OC(CCCC)CCCCC)CCCNC(=S)NC] |
| I 114 | 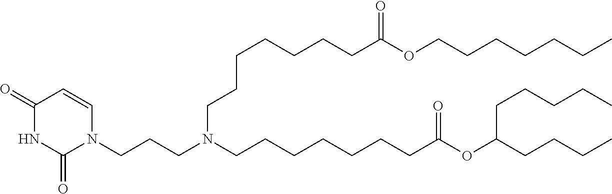 [SMILES: CCCCCCCOC(=O)CCCCCCCN(CCCCCCCC(=O)OC(CCCC)CCCCC)CCCn1ccc(=O)[nH]c1=O] |
| I 115 | 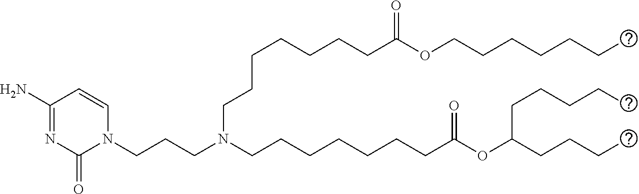 [SMILES: CCCCCCCOC(=O)CCCCCCCN(CCCCCCCC(=O)OC(CCCC)CCCCC)CCCn1ccc(N)nc1=O] |
| I 116 | 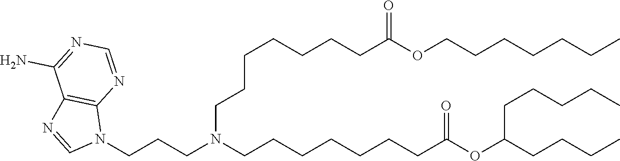 [SMILES: CCCCCCCOC(=O)CCCCCCCN(CCCCCCCC(=O)OC(CCCC)CCCCC)CCCn1cnc2c(N)ncnc21] |
| I 117 | 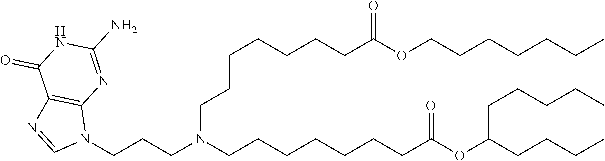 [SMILES: CCCCCCCOC(=O)CCCCCCCN(CCCCCCCC(=O)OC(CCCC)CCCCC)CCCn1cnc2c(=O)[nH]c(N)nc21] |
| I 118 | 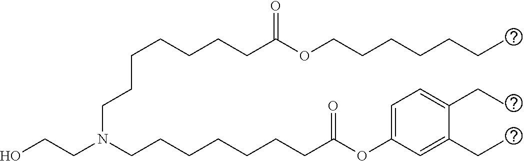 [SMILES: CCCCCCCOC(=O)CCCCCCCN(CCO)CCCCCCCC(=O)Oc1ccc(CC)c(CC)c1] |
| I 119 | 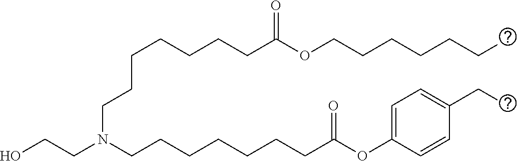 [SMILES: CCCCCCCOC(=O)CCCCCCCN(CCO)CCCCCCCC(=O)Oc1ccc(CC)cc1] |
| I 120 | 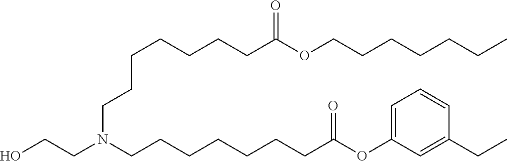 [SMILES: CCCCCCCOC(=O)CCCCCCCN(CCO)CCCCCCCC(=O)Oc1cccc(CC)c1] |
| I 121 | 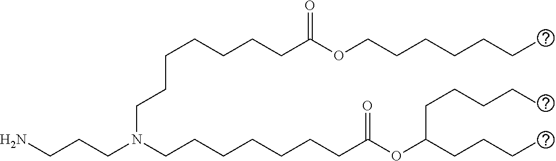 [SMILES: CCCCCCCOC(=O)CCCCCCCN(CCCN)CCCCCCCC(=O)OC(CCCC)CCCCC] |
| I 122 | 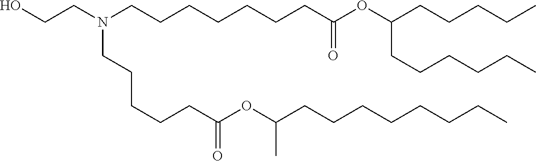 [SMILES: CCCCCCCCC(C)OC(=O)CCCCCN(CCO)CCCCCCCC(=O)OC(CCCCC)CCCCCC] |
| I 123 | 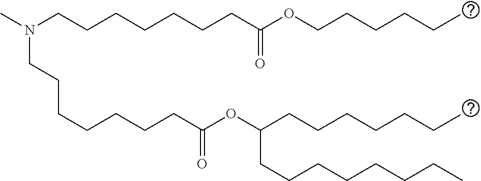 [SMILES: CCCCCCCCC(CCCCCCC)OC(=O)CCCCCCCN(C)CCCCCCCC(=O)OCCCCCC] |
| I 124 | 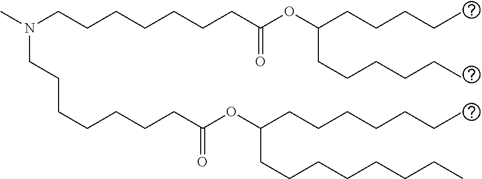 [SMILES: CCCCCCCCC(CCCCCCC)OC(=O)CCCCCCCN(C)CCCCCCCC(=O)OC(CCCCC)CCCCCC] |
| I 125 | 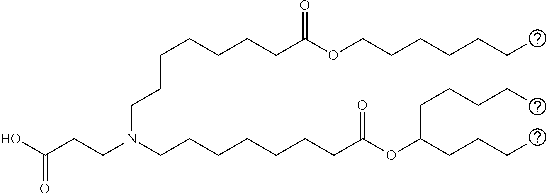 [SMILES: CCCCCCCOC(=O)CCCCCCCN(CCCCCCCC(=O)OC(CCCC)CCCCC)CCC(=O)O] |
| I 126 | 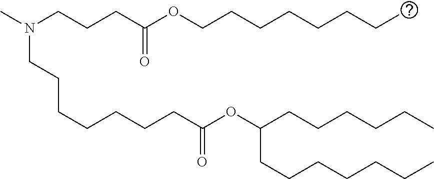 [SMILES: CCCCCCCCOC(=O)CCCN(C)CCCCCCCC(=O)OC(CCCCCC)CCCCCCC] |
| I 127 | 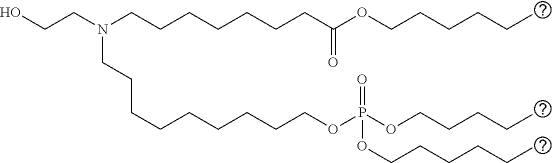 [SMILES: CCCCCCOC(=O)CCCCCCCN(CCO)CCCCCCCCCOP(=O)(OCCCCC)OCCCCCC] |
| I 128 | 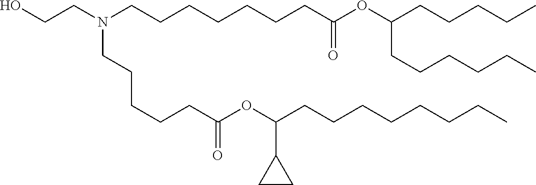 [SMILES: CCCCCCCCC(OC(=O)CCCCCN(CCO)CCCCCCCC(=O)OC(CCCCC)CCCCCC)C1CC1] |
| I 129 | 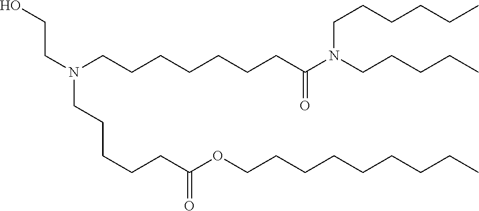 [SMILES: CCCCCCCCCOC(=O)CCCCCN(CCO)CCCCCCCC(=O)N(CCCCC)CCCCCC] |
| I 130 | 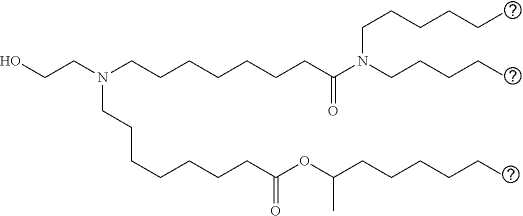 [SMILES: CCCCCCC(C)OC(=O)CCCCCCCN(CCO)CCCCCCCC(=O)N(CCCCC)CCCCCC] |
| I 131 | 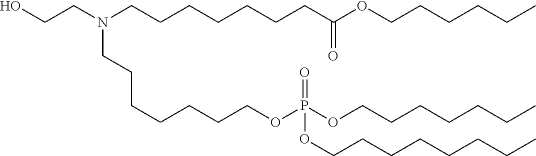 [SMILES: CCCCCCCCOP(=O)(OCCCCCCC)OCCCCCCCN(CCO)CCCCCCCC(=O)OCCCCCC] |
| I 132 | 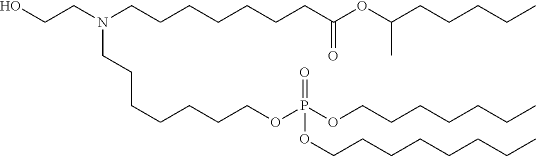 [SMILES: CCCCCCCCOP(=O)(OCCCCCCC)OCCCCCCCN(CCO)CCCCCCCC(=O)OC(C)CCCCC] |
| I 133 | 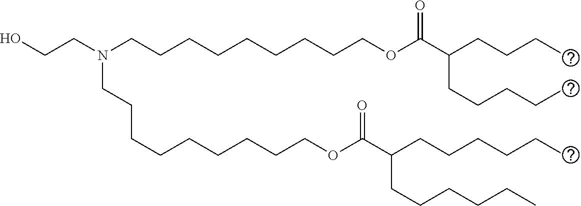 [SMILES: CCCCCCC(CCCCCC)C(=O)OCCCCCCCCCN(CCO)CCCCCCCCCOC(=O)C(CCCC)CCCCC] |
| I 134 | 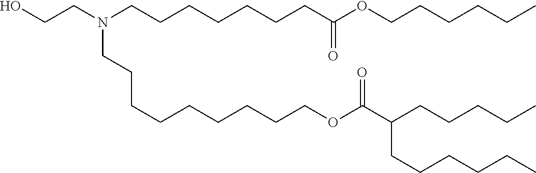 [SMILES: CCCCCCOC(=O)CCCCCCCN(CCO)CCCCCCCCCOC(=O)C(CCCCC)CCCCCC] |
| I 135 | 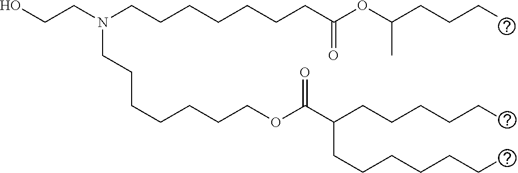 [SMILES: CCCCCCCC(CCCCCC)C(=O)OCCCCCCCN(CCO)CCCCCCCC(=O)OC(C)CCCC] |
| I 136 | 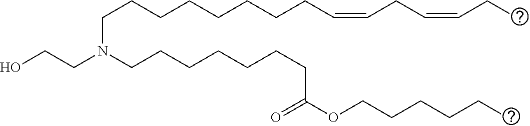 [SMILES: CC/C=C\C/C=C\CCCCCCCCN(CCO)CCCCCCCC(=O)OCCCCCC] |
| I 137 | 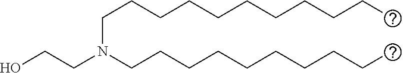 [SMILES: CCCCCCCCCCCN(CCO)CCCCCCCCCC] |
| I 138 | 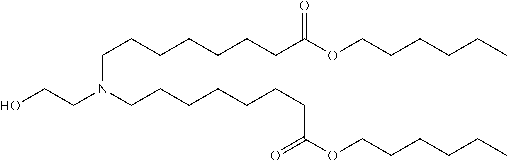 [SMILES: CCCCCCOC(=O)CCCCCCCN(CCO)CCCCCCCC(=O)OCCCCCC] |
| I 139 | 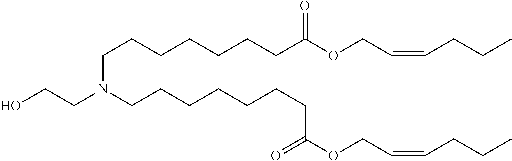 [SMILES: CCC/C=C\COC(=O)CCCCCCCN(CCO)CCCCCCCC(=O)OC/C=C\CCC] |
| I 140 | 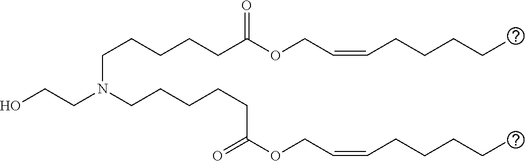 [SMILES: CCCCC/C=C\COC(=O)CCCCCN(CCO)CCCCCC(=O)OC/C=C\CCCCC] |
| I 141 | 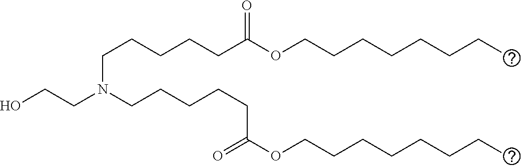 [SMILES: CCCCCCCCOC(=O)CCCCCN(CCO)CCCCCC(=O)OCCCCCCCC] |
| I 142 | 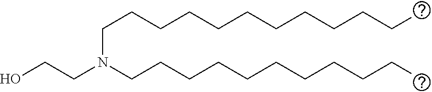 [SMILES: CCCCCCCCCCCCN(CCO)CCCCCCCCCCC] |
| I 143 | 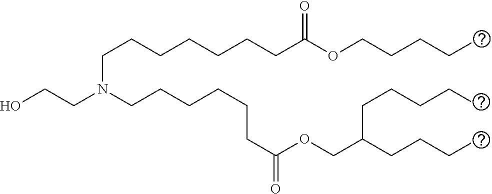 [SMILES: CCCCCOC(=O)CCCCCCCN(CCO)CCCCCCC(=O)OCC(CCCC)CCCCC] |
| I 144 | 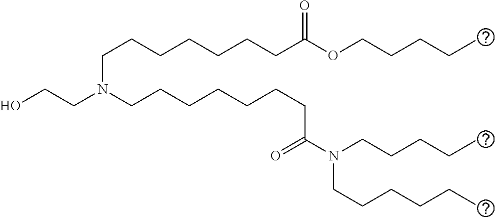 [SMILES: CCCCCCN(CCCCC)C(=O)CCCCCCCN(CCO)CCCCCCCC(=O)OCCCCC] |
| I 145 | 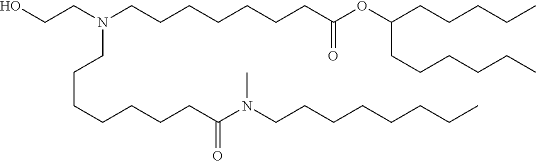 [SMILES: CCCCCCCCN(C)C(=O)CCCCCCCN(CCO)CCCCCCCC(=O)OC(CCCCC)CCCCCC] |
| I 146 | 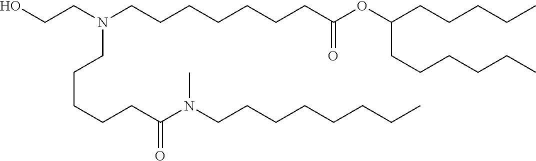 [SMILES: CCCCCCCCN(C)C(=O)CCCCCN(CCO)CCCCCCCC(=O)OC(CCCCC)CCCCCC] |
| I 147 | 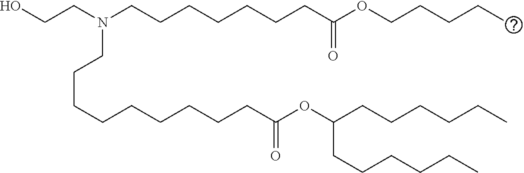 [SMILES: CCCCCCC(CCCCCC)OC(=O)CCCCCCCCCN(CCO)CCCCCCCC(=O)OCCCCC] |
| I 148 | 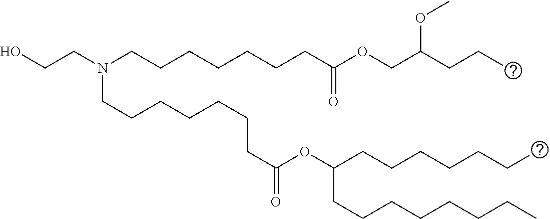 [SMILES: CCCCCCCCC(CCCCCCC)OC(=O)CCCCCCCN(CCO)CCCCCCCC(=O)OCC(CCC)OC] |
| I 149 | 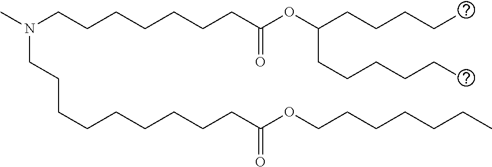 [SMILES: CCCCCCCOC(=O)CCCCCCCCCN(C)CCCCCCCC(=O)OC(CCCCC)CCCCCC] |
| I 150 | 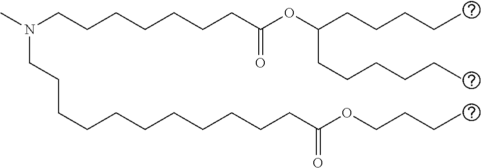 [SMILES: CCCCCCC(CCCCC)OC(=O)CCCCCCCN(C)CCCCCCCCCCCC(=O)OCCCC] |
| I 151 | 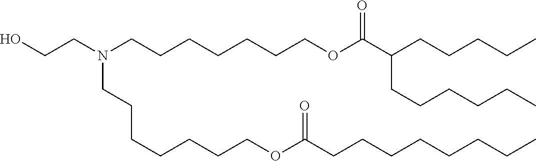 [SMILES: CCCCCCCCC(=O)OCCCCCCCN(CCO)CCCCCCCOC(=O)C(CCCCC)CCCCCC] |
| I 152 | 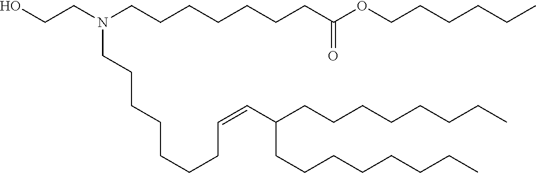 [SMILES: CCCCCCCCC(/C=C\CCCCCCCN(CCO)CCCCCCCC(=O)OCCCCCC)CCCCCCCC] |
| I 153 | 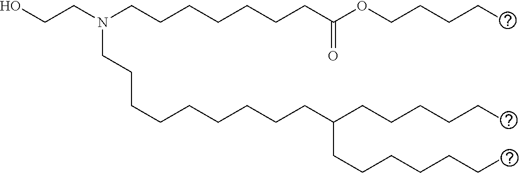 [SMILES: CCCCCCCC(CCCCCC)CCCCCCCCCN(CCO)CCCCCCCC(=O)OCCCCC] |
| I 154 | 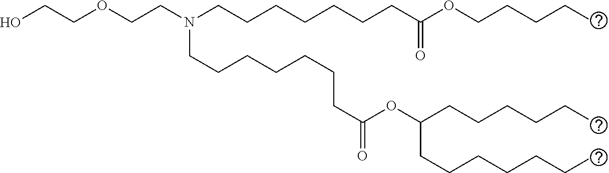 [SMILES: CCCCCCCC(CCCCCC)OC(=O)CCCCCCCN(CCCCCCCC(=O)OCCCCC)CCOCCO] |
| I 155 | 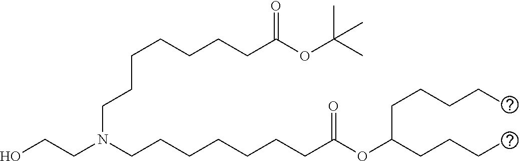 [SMILES: CCCCCC(CCCC)OC(=O)CCCCCCCN(CCO)CCCCCCCC(=O)OC(C)(C)C] |
| I 156 | 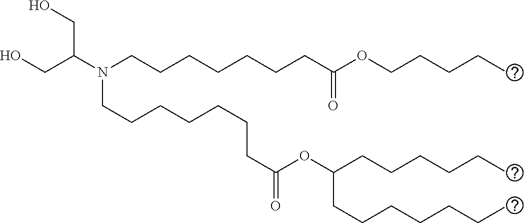 [SMILES: CCCCCCCC(CCCCCC)OC(=O)CCCCCCCN(CCCCCCCC(=O)OCCCCC)C(CO)CO] |
| I 157 | 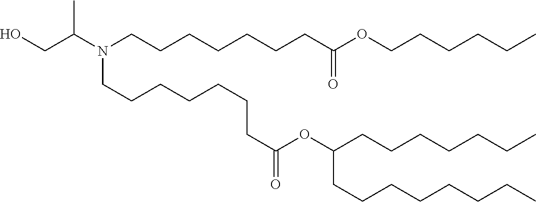 [SMILES: CCCCCCCCC(CCCCCCC)OC(=O)CCCCCCCN(CCCCCCCC(=O)OCCCCCC)C(C)CO] |
| I 158 | 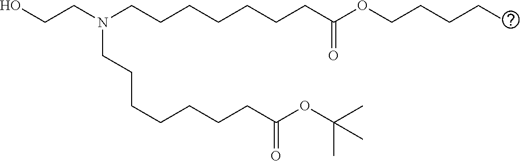 [SMILES: CCCCCOC(=O)CCCCCCCN(CCO)CCCCCCCC(=O)OC(C)(C)C] |
| I 159 | 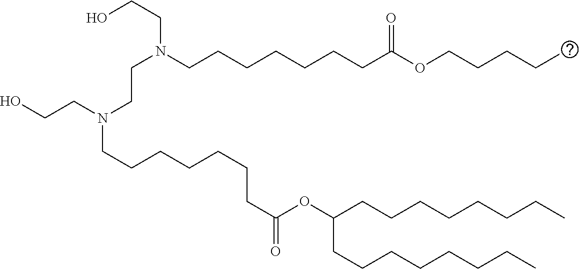 [SMILES: CCCCCCCCC(CCCCCCCC)OC(=O)CCCCCCCN(CCO)CCN(CCO)CCCCCCCC(=O)OCCCCC] |
| I 160 | 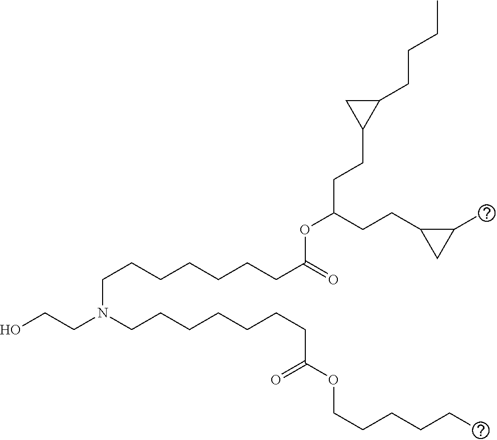 [SMILES: CCCCCCOC(=O)CCCCCCCN(CCO)CCCCCCCC(=O)OC(CCC1CC1C)CCC1CC1CCCC] |
| I 161 | 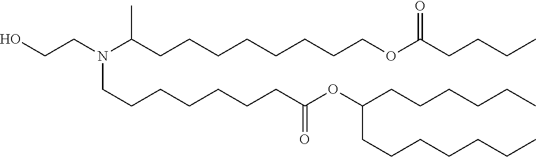 [SMILES: CCCCCCCC(CCCCCC)OC(=O)CCCCCCCN(CCO)C(C)CCCCCCCCOC(=O)CCCC] |
| I 162 | 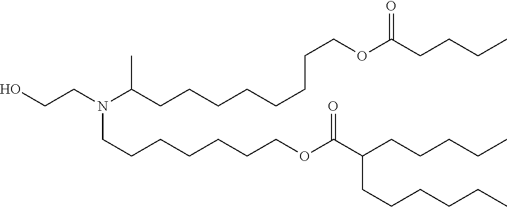 [SMILES: CCCCCCC(CCCCC)C(=O)OCCCCCCCN(CCO)C(C)CCCCCCCCOC(=O)CCCC] |
| I 163 | 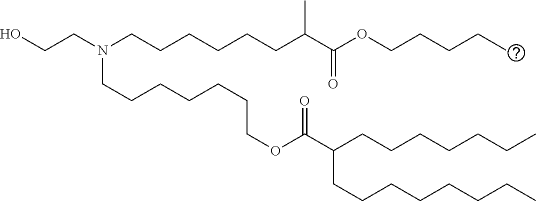 [SMILES: CCCCCCCCC(CCCCCCC)C(=O)OCCCCCCCN(CCO)CCCCCCC(C)C(=O)OCCCCC] |
| I 164 | 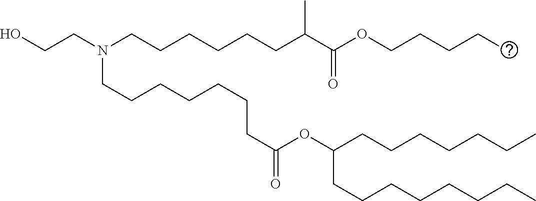 [SMILES: CCCCCCCCC(CCCCCCC)OC(=O)CCCCCCCN(CCO)CCCCCCC(C)C(=O)OCCCCC] |
| I 165 | 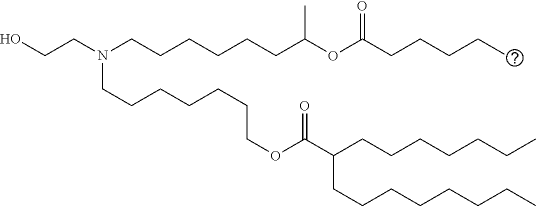 [SMILES: CCCCCCCCC(CCCCCCC)C(=O)OCCCCCCCN(CCO)CCCCCCC(C)OC(=O)CCCCC] |
| I 166 | 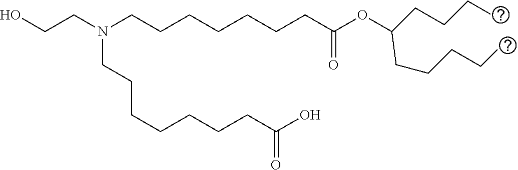 [SMILES: CCCCCC(CCCC)OC(=O)CCCCCCCN(CCO)CCCCCCCC(=O)O] |
| I 167 | 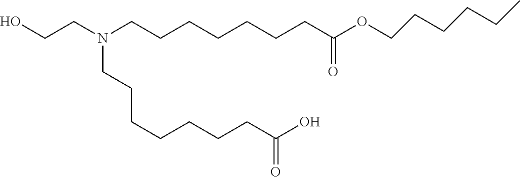 [SMILES: CCCCCCOC(=O)CCCCCCCN(CCO)CCCCCCCC(=O)O] |
| I 168 | 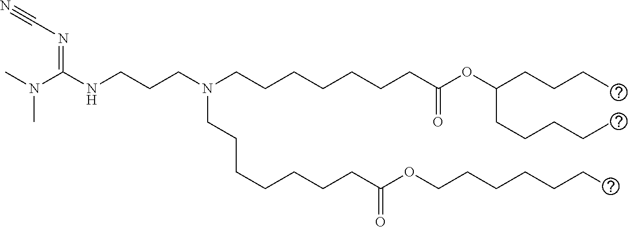 [SMILES: CCCCCCCOC(=O)CCCCCCCN(CCCCCCCC(=O)OC(CCCC)CCCCC)CCCN/C(=N/C#N)N(C)C] |
| I 169 | 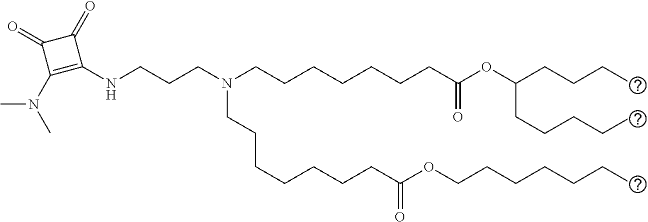 [SMILES: CCCCCCCOC(=O)CCCCCCCN(CCCCCCCC(=O)OC(CCCC)CCCCC)CCCNc1c(N(C)C)c(=O)c1=O] |
| I 170 | 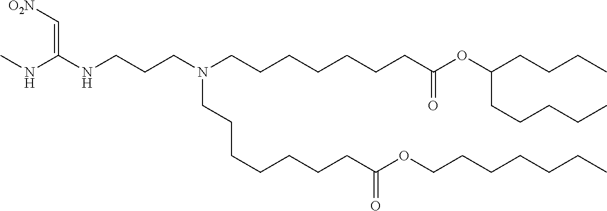 [SMILES: CCCCCCCOC(=O)CCCCCCCN(CCCCCCCC(=O)OC(CCCC)CCCCC)CCCN/C(=C/[N+](=O)[O-])NC] |
| I 171 | 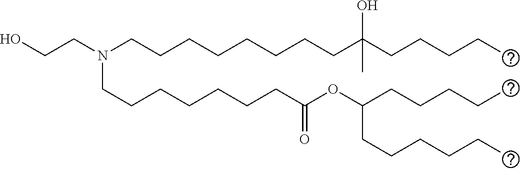 [SMILES: CCCCCCC(CCCCC)OC(=O)CCCCCCCN(CCO)CCCCCCCCC(C)(O)CCCCC] |
| I 172 | 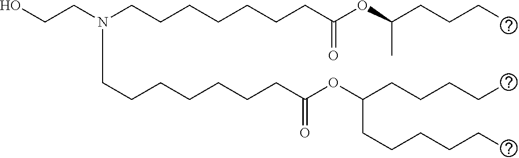 [SMILES: CCCCCCC(CCCCC)OC(=O)CCCCCCCN(CCO)CCCCCCCC(=O)O[C@H](C)CCCC] |
| I 173 | 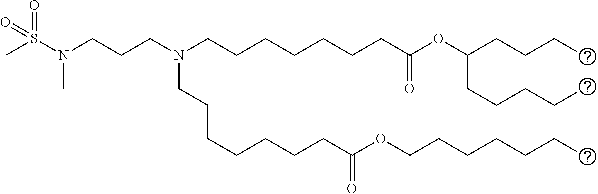 [SMILES: CCCCCCCOC(=O)CCCCCCCN(CCCCCCCC(=O)OC(CCCC)CCCCC)CCCN(C)S(C)(=O)=O] |
| I 174 | 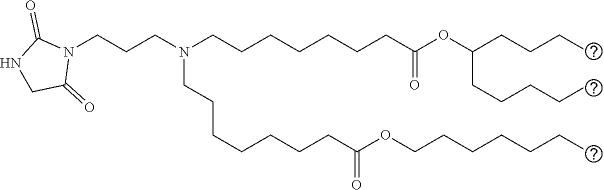 [SMILES: CCCCCCCOC(=O)CCCCCCCN(CCCCCCCC(=O)OC(CCCC)CCCCC)CCCN1C(=O)CNC1=O] |
| I 175 | 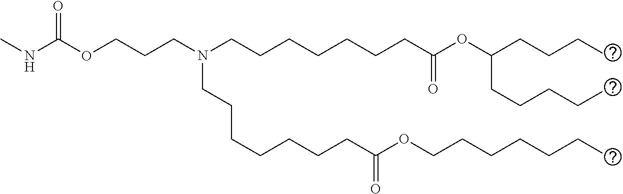 [SMILES: CCCCCCCOC(=O)CCCCCCCN(CCCCCCCC(=O)OC(CCCC)CCCCC)CCCOC(=O)NC] |
| I 176 | 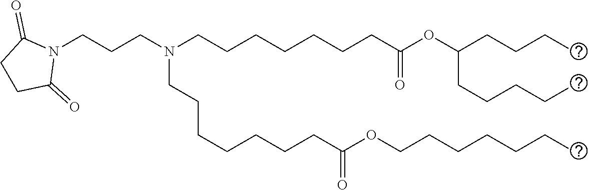 [SMILES: CCCCCCCOC(=O)CCCCCCCN(CCCCCCCC(=O)OC(CCCC)CCCCC)CCCN1C(=O)CCC1=O] |
| I 177 | 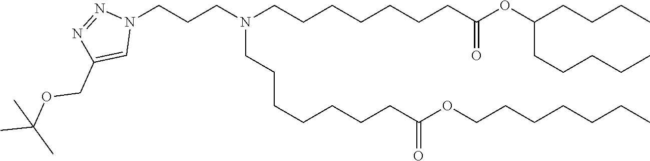 [SMILES: CCCCCCCOC(=O)CCCCCCCN(CCCCCCCC(=O)OC(CCCC)CCCCC)CCCn1cc(COC(C)(C)C)nn1] |
| I 178 | 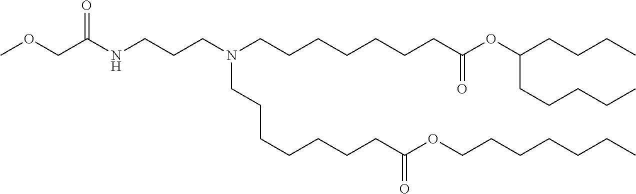 [SMILES: CCCCCCCOC(=O)CCCCCCCN(CCCCCCCC(=O)OC(CCCC)CCCCC)CCCNC(=O)COC] |
| I 179 | 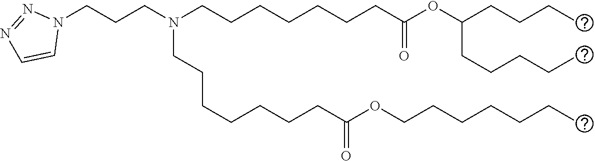 [SMILES: CCCCCCCOC(=O)CCCCCCCN(CCCCCCCC(=O)OC(CCCC)CCCCC)CCCn1ccnn1] |
| I 180 | 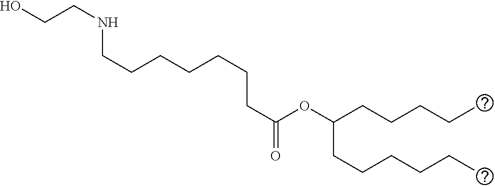 [SMILES: CCCCCCC(CCCCC)OC(=O)CCCCCCCNCCO] |
| I 181 | 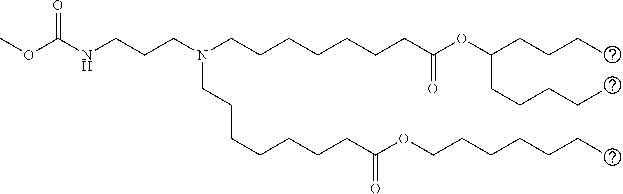 [SMILES: CCCCCCCOC(=O)CCCCCCCN(CCCCCCCC(=O)OC(CCCC)CCCCC)CCCNC(=O)OC] |
| I 182 | 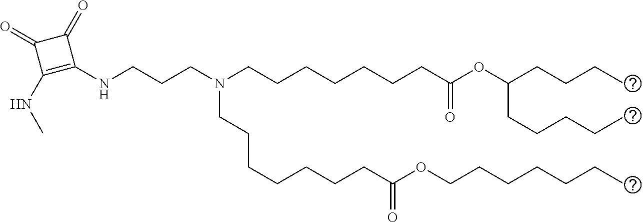 [SMILES: CCCCCCCOC(=O)CCCCCCCN(CCCCCCCC(=O)OC(CCCC)CCCCC)CCCNc1c(NC)c(=O)c1=O] |
| I 183 | 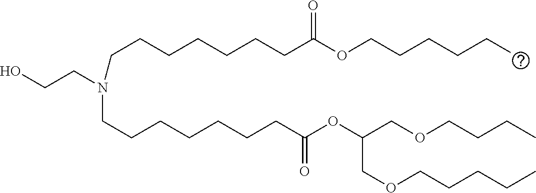 [SMILES: CCCCCCOC(=O)CCCCCCCN(CCO)CCCCCCCC(=O)OC(COCCCC)COCCCCC] |
| I 184 | 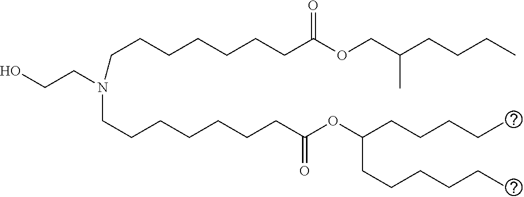 [SMILES: CCCCCCC(CCCCC)OC(=O)CCCCCCCN(CCO)CCCCCCCC(=O)OCC(C)CCCC] |
| I 185 | 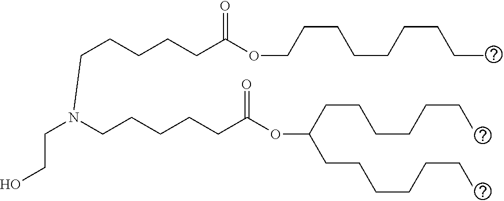 [SMILES: CCCCCCCCCOC(=O)CCCCCN(CCO)CCCCCC(=O)OC(CCCCCCC)CCCCCCC] |
| I 186 | 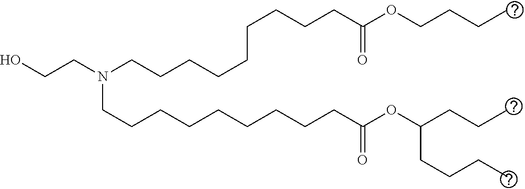 [SMILES: CCCCOC(=O)CCCCCCCCCN(CCO)CCCCCCCCCC(=O)OC(CCC)CCCC] |
| I 187 | 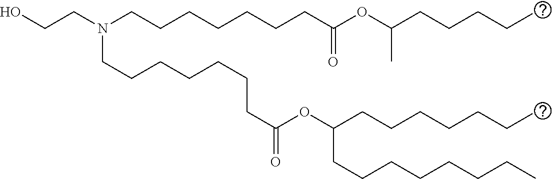 [SMILES: CCCCCCCCC(CCCCCCC)OC(=O)CCCCCCCN(CCO)CCCCCCCC(=O)OC(C)CCCCC] |
| I 188 | 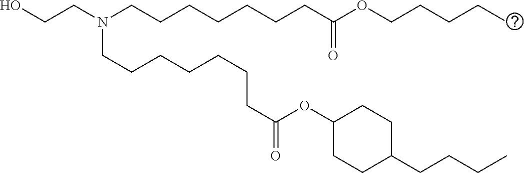 [SMILES: CCCCCOC(=O)CCCCCCCN(CCO)CCCCCCCC(=O)OC1CCC(CCCC)CC1] |
| I 189 | 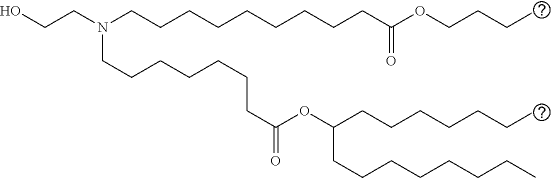 [SMILES: CCCCCCCCC(CCCCCCC)OC(=O)CCCCCCCN(CCO)CCCCCCCCCC(=O)OCCCC] |
| I 190 | 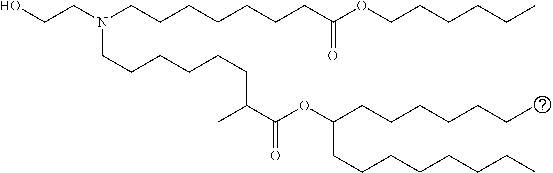 [SMILES: CCCCCCCCC(CCCCCCC)OC(=O)C(C)CCCCCCN(CCO)CCCCCCCC(=O)OCCCCCC] |
| I 191 | 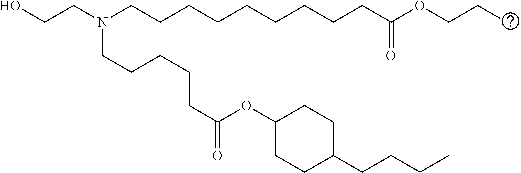 [SMILES: CCCCC1CCC(OC(=O)CCCCCN(CCO)CCCCCCCCCC(=O)OCCC)CC1] |
| I 192 | 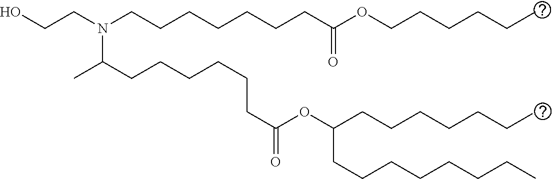 [SMILES: CCCCCCCCC(CCCCCCC)OC(=O)CCCCCCC(C)N(CCO)CCCCCCCC(=O)OCCCCCC] |
| I 193 | 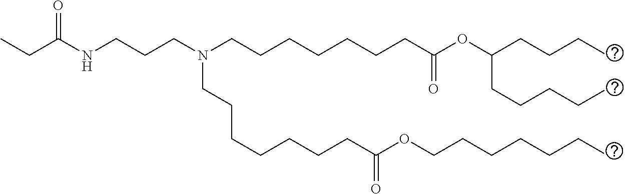 [SMILES: CCCCCCCOC(=O)CCCCCCCN(CCCCCCCC(=O)OC(CCCC)CCCCC)CCCNC(=O)CC] |
| I 194 | 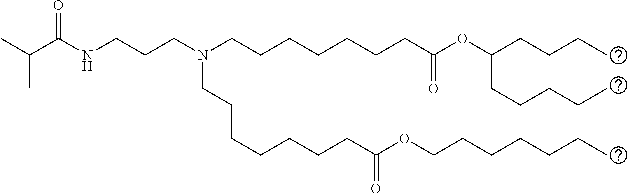 [SMILES: CCCCCCCOC(=O)CCCCCCCN(CCCCCCCC(=O)OC(CCCC)CCCCC)CCCNC(=O)C(C)C] |
| I 195 | 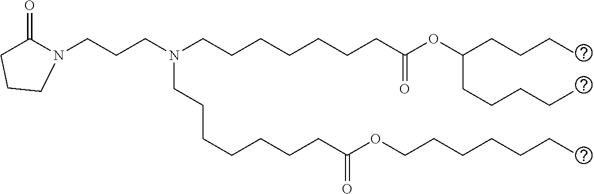 [SMILES: CCCCCCCOC(=O)CCCCCCCN(CCCCCCCC(=O)OC(CCCC)CCCCC)CCCN1CCCC1=O] |
| I 196 | 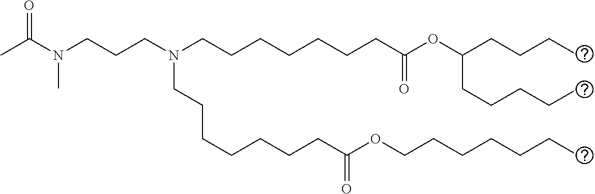 [SMILES: CCCCCCCOC(=O)CCCCCCCN(CCCCCCCC(=O)OC(CCCC)CCCCC)CCCN(C)C(C)=O] |
| I 197 | 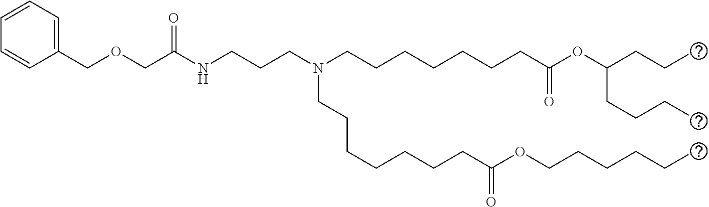 [SMILES: CCCCCCOC(=O)CCCCCCCN(CCCCCCCC(=O)OC(CCC)CCCC)CCCNC(=O)COCc1ccccc1] |
| I 198 | 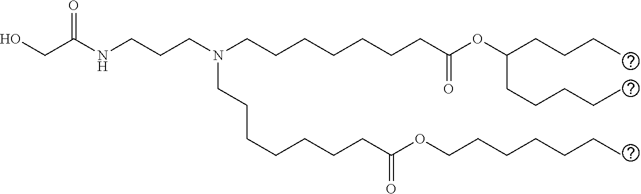 [SMILES: CCCCCCCOC(=O)CCCCCCCN(CCCCCCCC(=O)OC(CCCC)CCCCC)CCCNC(=O)CO] |
| I 199 | 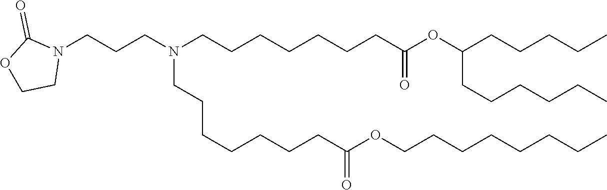 [SMILES: CCCCCCCCOC(=O)CCCCCCCN(CCCCCCCC(=O)OC(CCCCC)CCCCCC)CCCN1CCOC1=O] |
| I 200 | 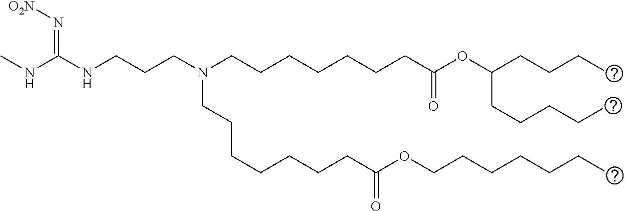 [SMILES: CCCCCCCOC(=O)CCCCCCCN(CCCCCCCC(=O)OC(CCCC)CCCCC)CCCN/C(=N/[N+](=O)[O-])NC] |
| I 201 | 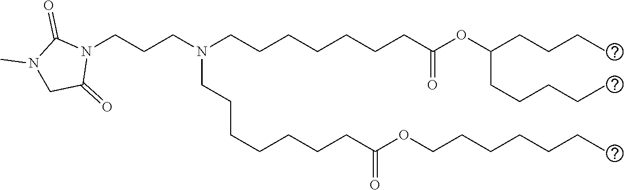 [SMILES: CCCCCCCOC(=O)CCCCCCCN(CCCCCCCC(=O)OC(CCCC)CCCCC)CCCN1C(=O)CN(C)C1=O] |
| I 202 | 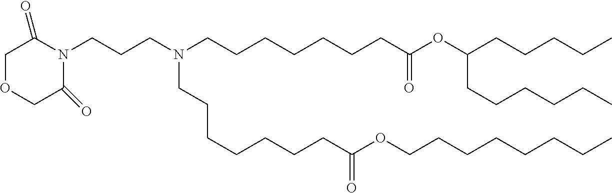 [SMILES: CCCCCCCCOC(=O)CCCCCCCN(CCCCCCCC(=O)OC(CCCCC)CCCCCC)CCCN1C(=O)COCC1=O] |
| I 203 | 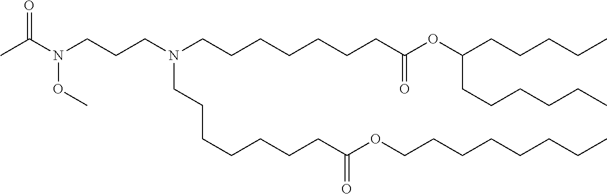 [SMILES: CCCCCCCCOC(=O)CCCCCCCN(CCCCCCCC(=O)OC(CCCCC)CCCCCC)CCCN(OC)C(C)=O] |
| I 204 | 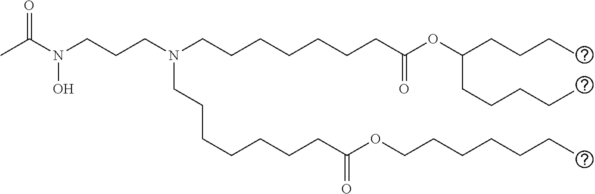 [SMILES: CCCCCCCOC(=O)CCCCCCCN(CCCCCCCC(=O)OC(CCCC)CCCCC)CCCN(O)C(C)=O] |
| I 205 | 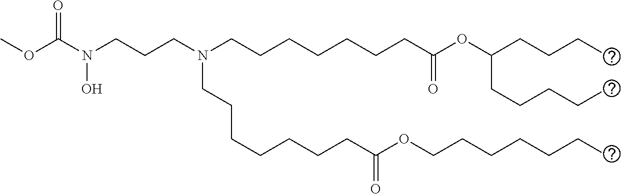 [SMILES: CCCCCCCOC(=O)CCCCCCCN(CCCCCCCC(=O)OC(CCCC)CCCCC)CCCN(O)C(=O)OC] |
| I 206 | 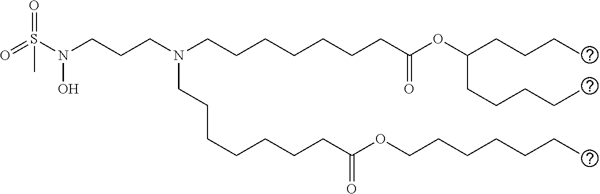 [SMILES: CCCCCCCOC(=O)CCCCCCCN(CCCCCCCC(=O)OC(CCCC)CCCCC)CCCN(O)S(C)(=O)=O] |
| I 207 | 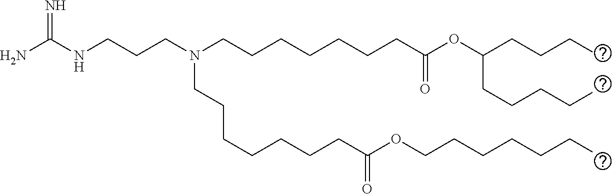 [SMILES: CCCCCCCOC(=O)CCCCCCCN(CCCCCCCC(=O)OC(CCCC)CCCCC)CCCNC(=N)N] |
| I 208 | 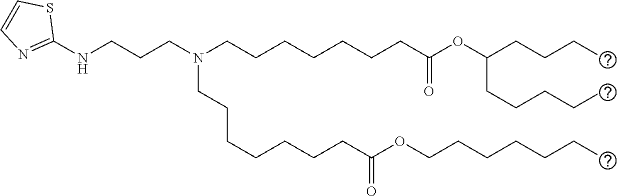 [SMILES: CCCCCCCOC(=O)CCCCCCCN(CCCCCCCC(=O)OC(CCCC)CCCCC)CCCNc1nccs1] |
| I 209 | 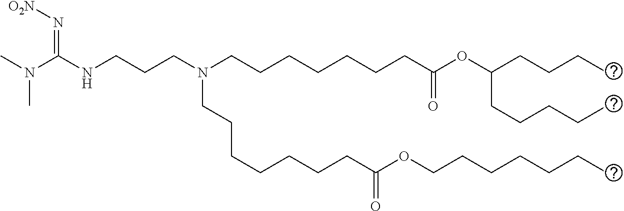 [SMILES: CCCCCCCOC(=O)CCCCCCCN(CCCCCCCC(=O)OC(CCCC)CCCCC)CCCN/C(=N/[N+](=O)[O-])N(C)C] |
| I 210 | 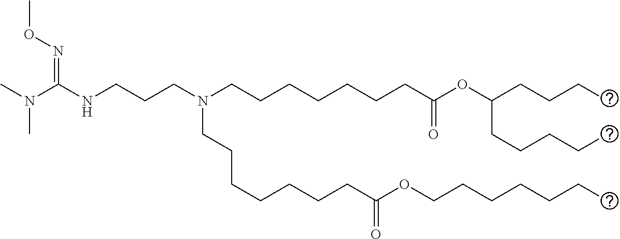 [SMILES: CCCCCCCOC(=O)CCCCCCCN(CCCCCCCC(=O)OC(CCCC)CCCCC)CCCN/C(=N/OC)N(C)C] |
| I 211 | 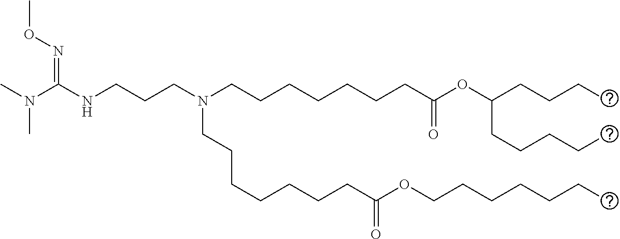 [SMILES: CCCCCCCOC(=O)CCCCCCCN(CCCCCCCC(=O)OC(CCCC)CCCCC)CCCN/C(=N/OC)N(C)C] |
| I 212 | 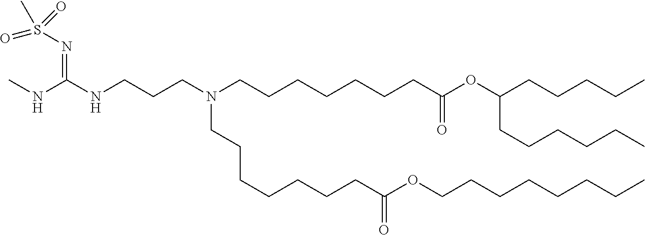 [SMILES: CCCCCCCCOC(=O)CCCCCCCN(CCCCCCCC(=O)OC(CCCCC)CCCCCC)CCCN/C(=N/S(C)(=O)=O)NC] |
| I 213 | 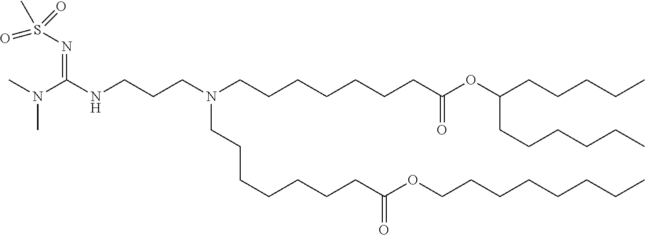 [SMILES: CCCCCCCCOC(=O)CCCCCCCN(CCCCCCCC(=O)OC(CCCCC)CCCCCC)CCCN/C(=N/S(C)(=O)=O)N(C)C] |
| I 214 | 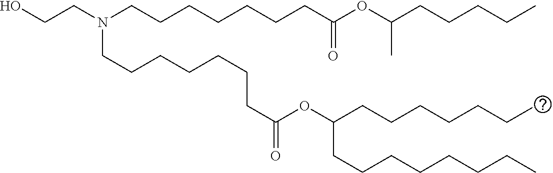 [SMILES: CCCCCCCCC(CCCCCCC)OC(=O)CCCCCCCN(CCO)CCCCCCCC(=O)OC(C)CCCCC] |
| I 215 | 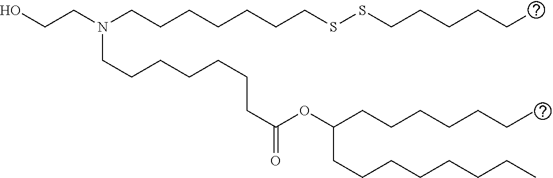 [SMILES: CCCCCCCCC(CCCCCCC)OC(=O)CCCCCCCN(CCO)CCCCCCCSSCCCCCC] |
| I 216 | 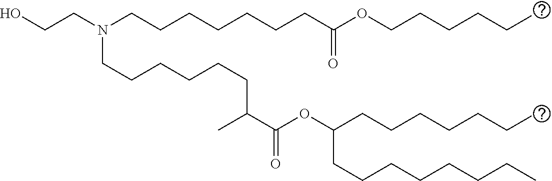 [SMILES: CCCCCCCCC(CCCCCCC)OC(=O)C(C)CCCCCCN(CCO)CCCCCCCC(=O)OCCCCCC] |
| I 217 | 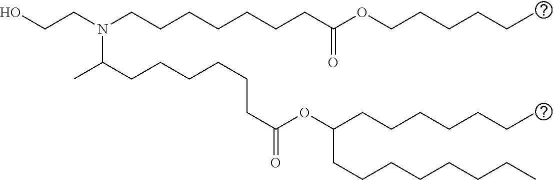 [SMILES: CCCCCCCCC(CCCCCCC)OC(=O)CCCCCCC(C)N(CCO)CCCCCCCC(=O)OCCCCCC] |
| I 218 | 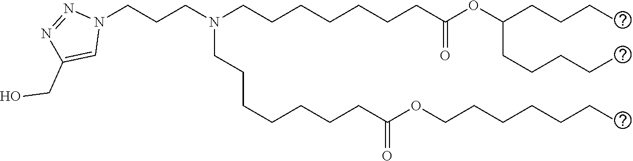 [SMILES: CCCCCCCOC(=O)CCCCCCCN(CCCCCCCC(=O)OC(CCCC)CCCCC)CCCn1cc(CO)nn1] |
| I 219 | 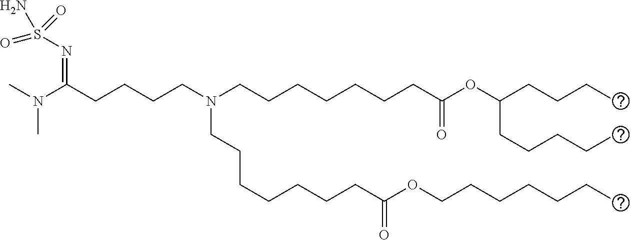 [SMILES: CCCCCCCOC(=O)CCCCCCCN(CCCCCCCC(=O)OC(CCCC)CCCCC)CCCC/C(=N/S(N)(=O)=O)N(C)C] |
| I 220 | 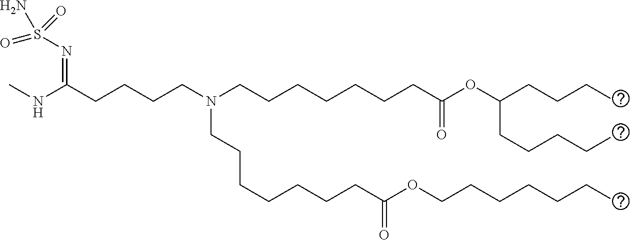 [SMILES: CCCCCCCOC(=O)CCCCCCCN(CCCCCCCC(=O)OC(CCCC)CCCCC)CCCC/C(=N/S(N)(=O)=O)NC] |
| I 221 | 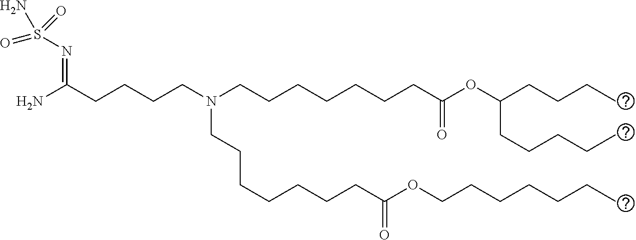 [SMILES: CCCCCCCOC(=O)CCCCCCCN(CCCCCCCC(=O)OC(CCCC)CCCCC)CCCC/C(N)=N/S(N)(=O)=O] |
| I 222 | 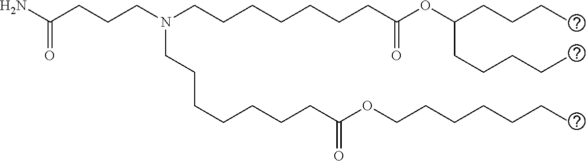 [SMILES: CCCCCCCOC(=O)CCCCCCCN(CCCCCCCC(=O)OC(CCCC)CCCCC)CCCC(N)=O] |
| I 223 | 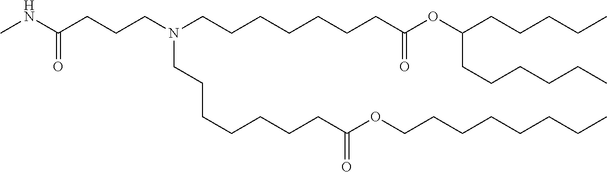 [SMILES: CCCCCCCCOC(=O)CCCCCCCN(CCCCCCCC(=O)OC(CCCCC)CCCCCC)CCCC(=O)NC] |
| I 224 | 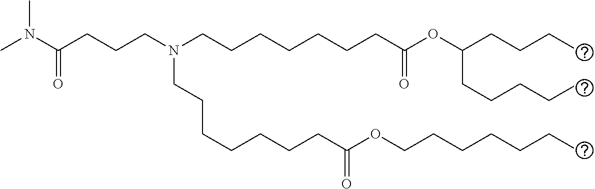 [SMILES: CCCCCCCOC(=O)CCCCCCCN(CCCCCCCC(=O)OC(CCCC)CCCCC)CCCC(=O)N(C)C] |
| I 225 | 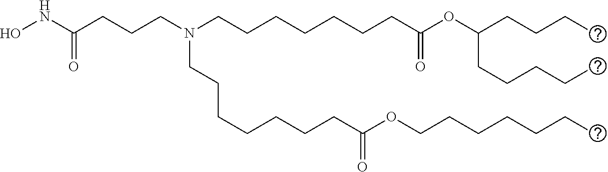 [SMILES: CCCCCCCOC(=O)CCCCCCCN(CCCCCCCC(=O)OC(CCCC)CCCCC)CCCC(=O)NO] |
| I 226 | 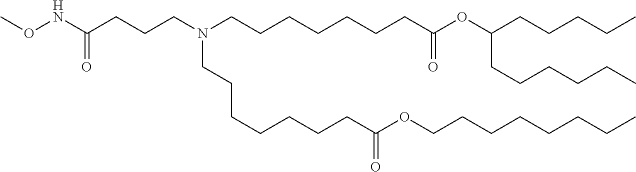 [SMILES: CCCCCCCCOC(=O)CCCCCCCN(CCCCCCCC(=O)OC(CCCCC)CCCCCC)CCCC(=O)NOC] |
| I 227 | 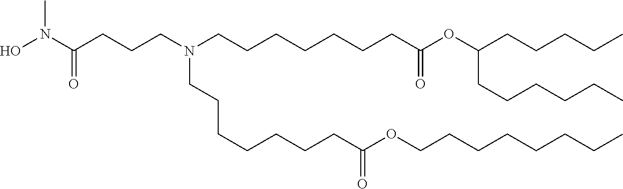 [SMILES: CCCCCCCCOC(=O)CCCCCCCN(CCCCCCCC(=O)OC(CCCCC)CCCCCC)CCCC(=O)N(C)O] |
| I 228 | 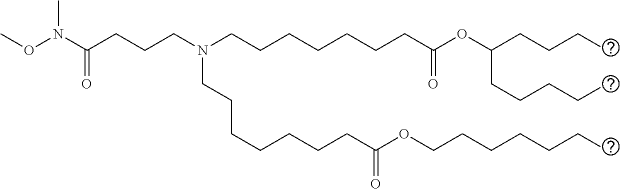 [SMILES: CCCCCCCOC(=O)CCCCCCCN(CCCCCCCC(=O)OC(CCCC)CCCCC)CCCC(=O)N(C)OC] |
| I 229 | 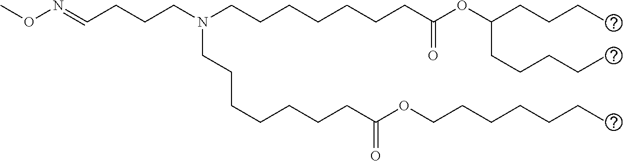 [SMILES: CCCCCCCOC(=O)CCCCCCCN(CCC/C=N/OC)CCCCCCCC(=O)OC(CCCC)CCCCC] |
| I 230 | 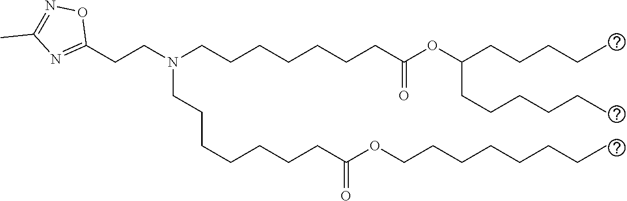 [SMILES: CCCCCCCCOC(=O)CCCCCCCN(CCCCCCCC(=O)OC(CCCCC)CCCCCC)CCc1nc(C)no1] |
| I 231 | 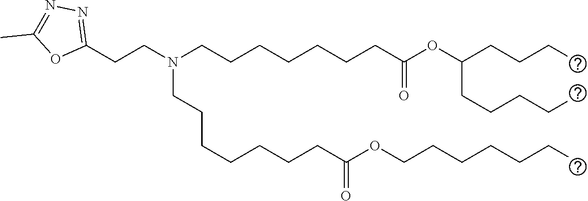 [SMILES: CCCCCCCOC(=O)CCCCCCCN(CCCCCCCC(=O)OC(CCCC)CCCCC)CCc1nnc(C)o1] |
| I 232 | 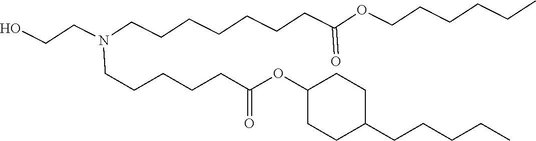 [SMILES: CCCCCCOC(=O)CCCCCCCN(CCO)CCCCCC(=O)OC1CCC(CCCCC)CC1] |
| I 233 | 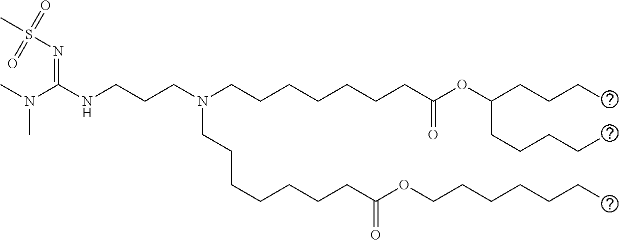 [SMILES: CCCCCCCOC(=O)CCCCCCCN(CCCCCCCC(=O)OC(CCCC)CCCCC)CCCN/C(=N/S(C)(=O)=O)N(C)C] |
| I 234 | 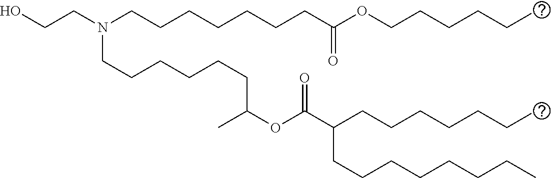 [SMILES: CCCCCCCCC(CCCCCCC)C(=O)OC(C)CCCCCCN(CCO)CCCCCCCC(=O)OCCCCCC] |
| I 235 | 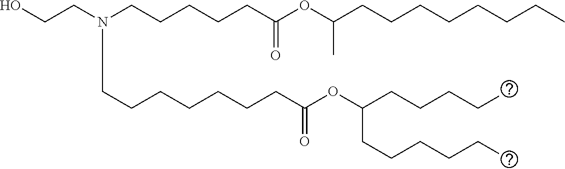 [SMILES: CCCCCCCCC(C)OC(=O)CCCCCN(CCO)CCCCCCCC(=O)OC(CCCCC)CCCCCC] |
| I 236 | 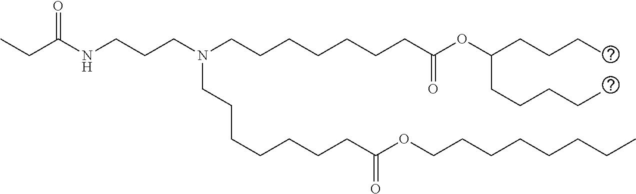 [SMILES: CCCCCCCCOC(=O)CCCCCCCN(CCCCCCCC(=O)OC(CCCC)CCCCC)CCCNC(=O)CC] |
| I 237 | 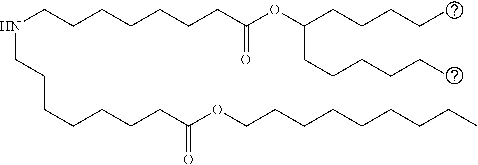 [SMILES: CCCCCCCCCOC(=O)CCCCCCCNCCCCCCCC(=O)OC(CCCCC)CCCCCC] |
| I 238 | 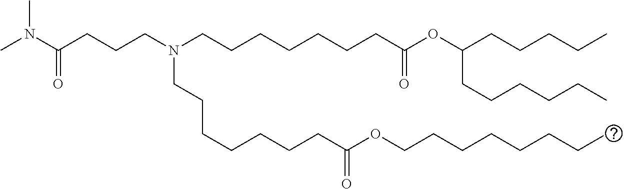 [SMILES: CCCCCCCCOC(=O)CCCCCCCN(CCCCCCCC(=O)OC(CCCCC)CCCCCC)CCCC(=O)N(C)C] |
| I 239 | 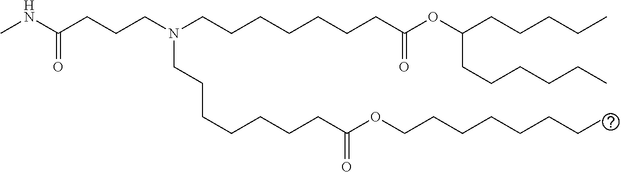 [SMILES: CCCCCCCCOC(=O)CCCCCCCN(CCCCCCCC(=O)OC(CCCCC)CCCCCC)CCCC(=O)NC] |
| I 240 | 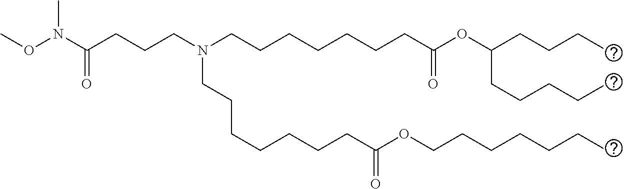 [SMILES: CCCCCCCOC(=O)CCCCCCCN(CCCCCCCC(=O)OC(CCCC)CCCCC)CCCC(=O)N(C)OC] |
| I 241 | 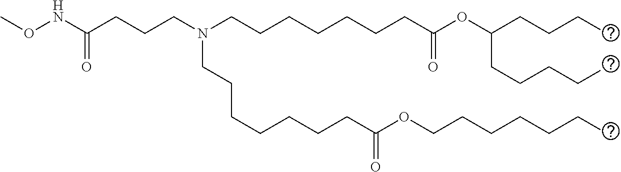 [SMILES: CCCCCCCOC(=O)CCCCCCCN(CCCCCCCC(=O)OC(CCCC)CCCCC)CCCC(=O)NOC] |
| I 242 | 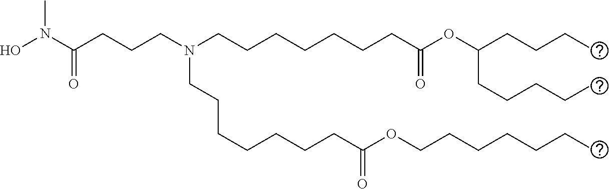 [SMILES: CCCCCCCOC(=O)CCCCCCCN(CCCCCCCC(=O)OC(CCCC)CCCCC)CCCC(=O)N(C)O] |
| I 243 | 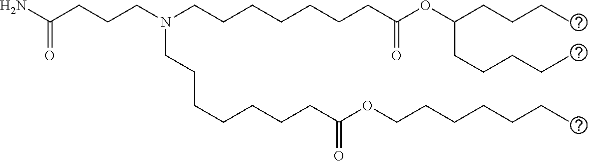 [SMILES: CCCCCCCOC(=O)CCCCCCCN(CCCCCCCC(=O)OC(CCCC)CCCCC)CCCC(N)=O] |
| I 244 | 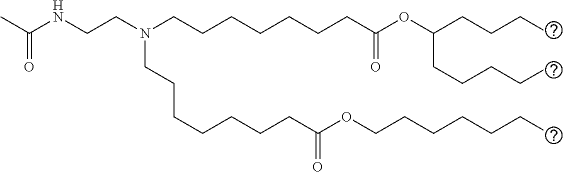 [SMILES: CCCCCCCOC(=O)CCCCCCCN(CCCCCCCC(=O)OC(CCCC)CCCCC)CCNC(C)=O] |
| I 245 | 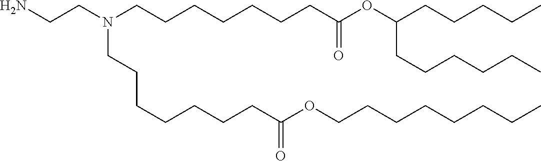 [SMILES: CCCCCCCCOC(=O)CCCCCCCN(CCN)CCCCCCCC(=O)OC(CCCCC)CCCCCC] |
| I 246 | 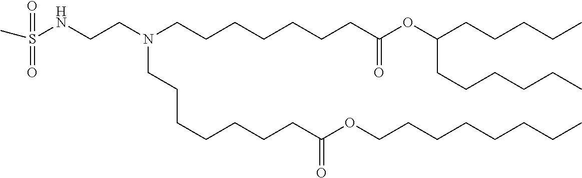 [SMILES: CCCCCCCCOC(=O)CCCCCCCN(CCCCCCCC(=O)OC(CCCCC)CCCCCC)CCNS(C)(=O)=O] |
| I 247 | 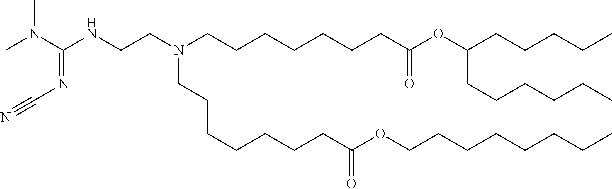 [SMILES: CCCCCCCCOC(=O)CCCCCCCN(CCCCCCCC(=O)OC(CCCCC)CCCCCC)CCN/C(=N/C#N)N(C)C] |
| I 248 | 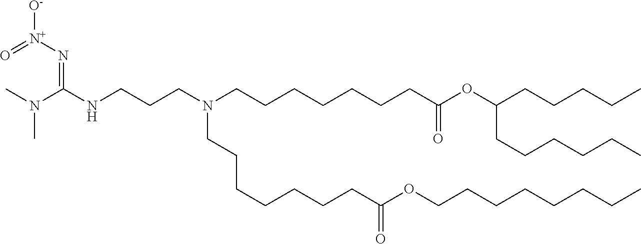 [SMILES: CCCCCCCCOC(=O)CCCCCCCN(CCCCCCCC(=O)OC(CCCCC)CCCCCC)CCCN/C(=N/[N+](=O)[O-])N(C)C] |
| I 249 | 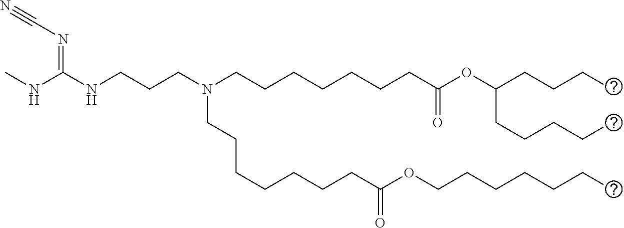 [SMILES: CCCCCCCOC(=O)CCCCCCCN(CCCCCCCC(=O)OC(CCCC)CCCCC)CCCN/C(=N/C#N)NC] |
| I 250 | 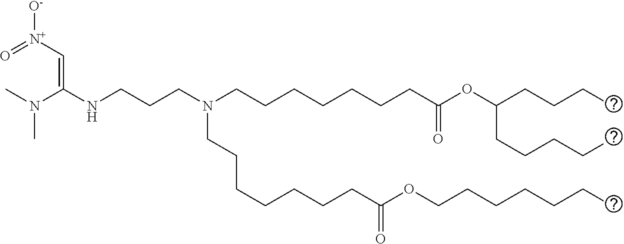 [SMILES: CCCCCCCOC(=O)CCCCCCCN(CCCCCCCC(=O)OC(CCCC)CCCCC)CCCN/C(=C/[N+](=O)[O-])N(C)C] |
| I 251 | 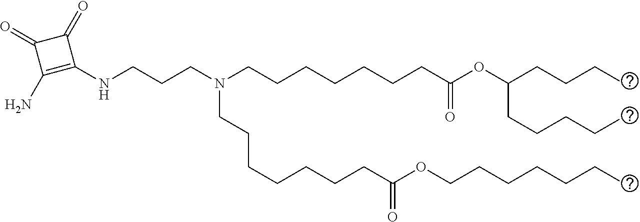 [SMILES: CCCCCCCOC(=O)CCCCCCCN(CCCCCCCC(=O)OC(CCCC)CCCCC)CCCNc1c(N)c(=O)c1=O] |
| I 252 | 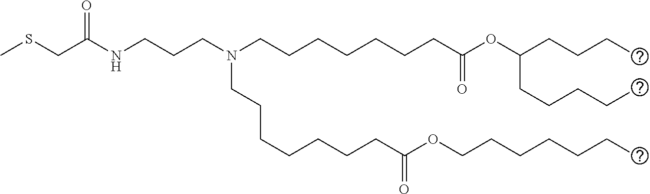 [SMILES: CCCCCCCOC(=O)CCCCCCCN(CCCCCCCC(=O)OC(CCCC)CCCCC)CCCNC(=O)CSC] |
| I 253 | 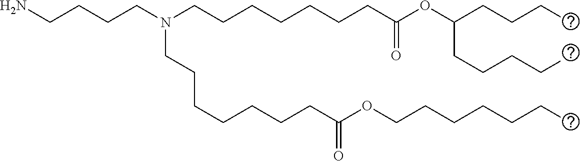 [SMILES: CCCCCCCOC(=O)CCCCCCCN(CCCCN)CCCCCCCC(=O)OC(CCCC)CCCCC] |
| I 254 | 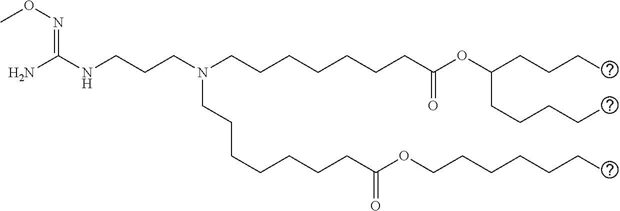 [SMILES: CCCCCCCOC(=O)CCCCCCCN(CCCCCCCC(=O)OC(CCCC)CCCCC)CCCN/C(N)=N/OC] |
| I 255 | 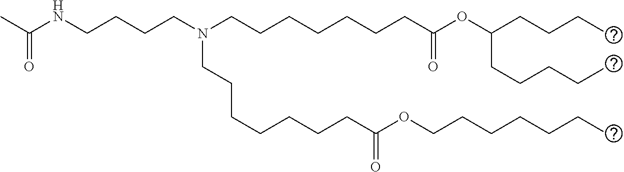 [SMILES: CCCCCCCOC(=O)CCCCCCCN(CCCCCCCC(=O)OC(CCCC)CCCCC)CCCCNC(C)=O] |
| I 256 | 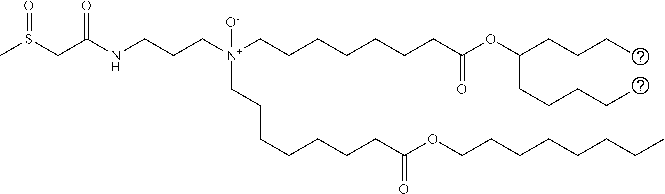 [SMILES: CCCCCCCCOC(=O)CCCCCCC[N+]([O-])(CCCCCCCC(=O)OC(CCCC)CCCCC)CCCNC(=O)CS(C)=O] |
| I 257 | 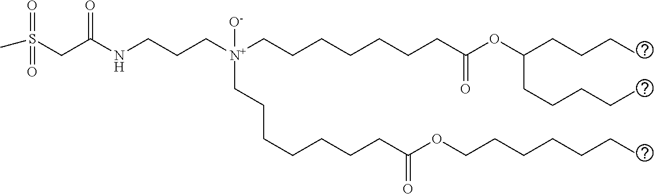 [SMILES: CCCCCCCOC(=O)CCCCCCC[N+]([O-])(CCCCCCCC(=O)OC(CCCC)CCCCC)CCCNC(=O)CS(C)(=O)=O] |
| I 258 | 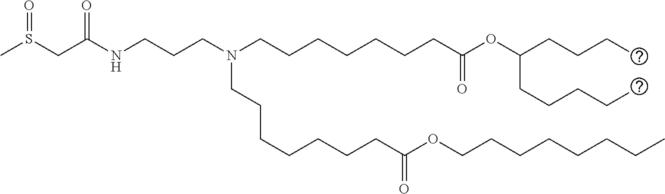 [SMILES: CCCCCCCCOC(=O)CCCCCCCN(CCCCCCCC(=O)OC(CCCC)CCCCC)CCCNC(=O)CS(C)=O] |
| I 259 | 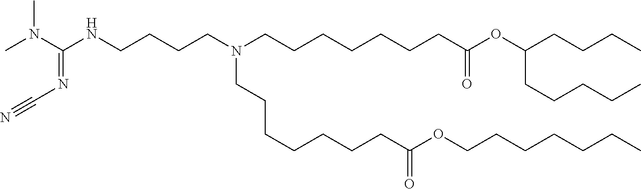 [SMILES: CCCCCCCOC(=O)CCCCCCCN(CCCCCCCC(=O)OC(CCCC)CCCCC)CCCCN/C(=N/C#N)N(C)C] |
| I 260 | 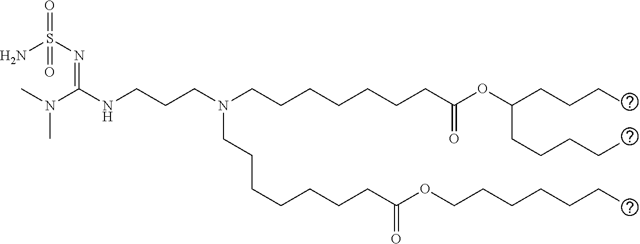 [SMILES: CCCCCCCOC(=O)CCCCCCCN(CCCCCCCC(=O)OC(CCCC)CCCCC)CCCN/C(=N/S(N)(=O)=O)N(C)C] |
| I 261 | 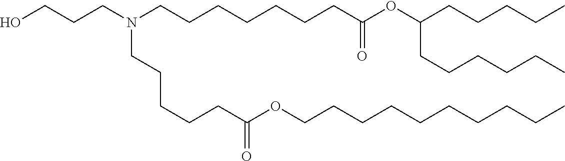 [SMILES: CCCCCCCCCCOC(=O)CCCCCN(CCCO)CCCCCCCC(=O)OC(CCCCC)CCCCCC] |
| I 262 | 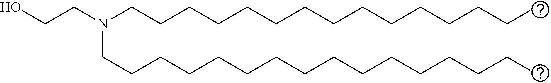 [SMILES: CCCCCCCCCCCCCCCCN(CCO)CCCCCCCCCCCCCCC] |
| I 263 | 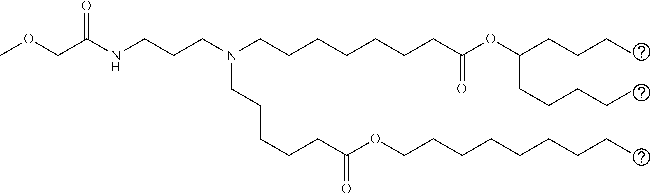 [SMILES: CCCCCCCCCOC(=O)CCCCCN(CCCCCCCC(=O)OC(CCCC)CCCCC)CCCNC(=O)COC] |
| I 264 | 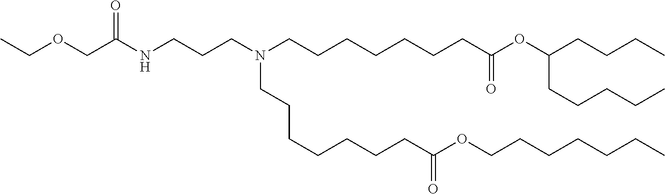 [SMILES: CCCCCCCOC(=O)CCCCCCCN(CCCCCCCC(=O)OC(CCCC)CCCCC)CCCNC(=O)COCC] |
| I 265 | 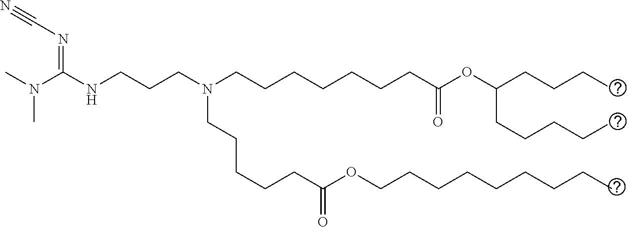 [SMILES: CCCCCCCCCOC(=O)CCCCCN(CCCCCCCC(=O)OC(CCCC)CCCCC)CCCN/C(=N/C#N)N(C)C] |
| I 266 | 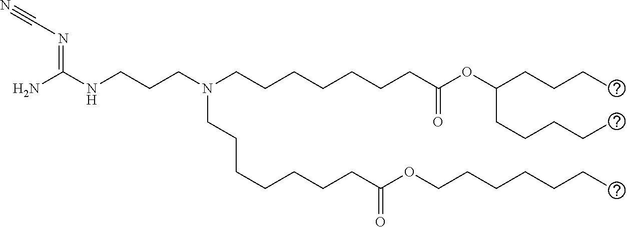 [SMILES: CCCCCCCOC(=O)CCCCCCCN(CCCCCCCC(=O)OC(CCCC)CCCCC)CCCN/C(N)=N/C#N] |
| I 267 | 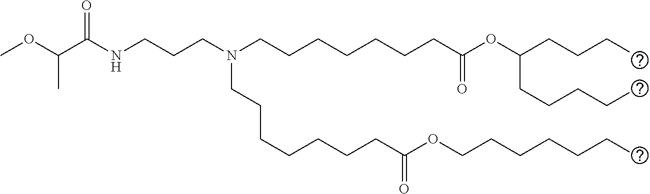 [SMILES: CCCCCCCOC(=O)CCCCCCCN(CCCCCCCC(=O)OC(CCCC)CCCCC)CCCNC(=O)C(C)OC] |
| I 268 | 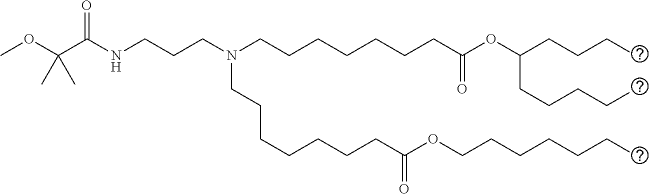 [SMILES: CCCCCCCOC(=O)CCCCCCCN(CCCCCCCC(=O)OC(CCCC)CCCCC)CCCNC(=O)C(C)(C)OC] |
| I 269 | 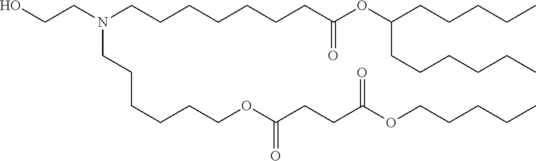 [SMILES: CCCCCCC(CCCCC)OC(=O)CCCCCCCN(CCO)CCCCCCOC(=O)CCC(=O)OCCCCC] |
| I 270 | 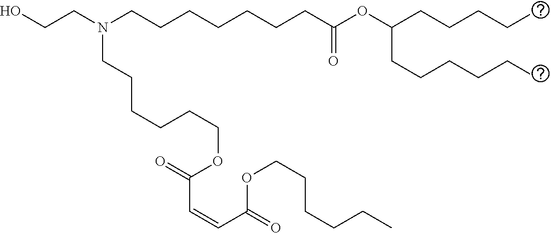 [SMILES: CCCCCCOC(=O)/C=C\C(=O)OCCCCCCN(CCO)CCCCCCCC(=O)OC(CCCCC)CCCCCC] |
| I 271 | 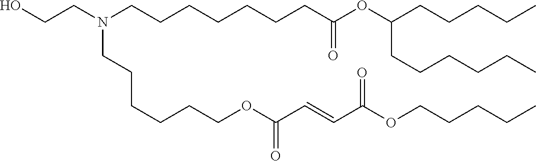 [SMILES: CCCCCCC(CCCCC)OC(=O)CCCCCCCN(CCO)CCCCCCOC(=O)/C=C/C(=O)OCCCCC] |
| I 272 | 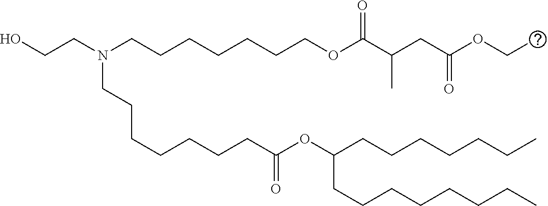 [SMILES: CCCCCCCCC(CCCCCCC)OC(=O)CCCCCCCN(CCO)CCCCCCCOC(=O)C(C)CC(=O)OCC] |
| I 273 | 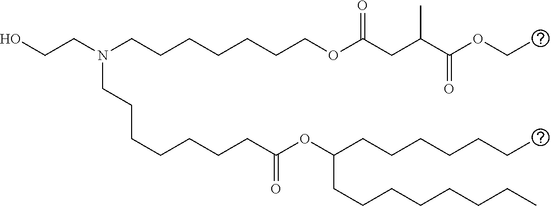 [SMILES: CCCCCCCCC(CCCCCCC)OC(=O)CCCCCCCN(CCO)CCCCCCCOC(=O)CC(C)C(=O)OCC] |
| I 274 | 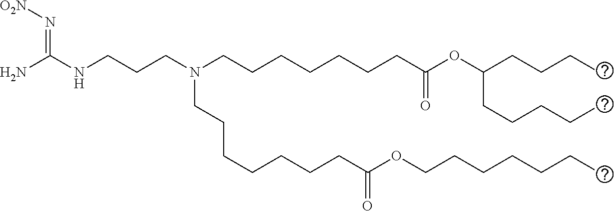 [SMILES: CCCCCCCOC(=O)CCCCCCCN(CCCCCCCC(=O)OC(CCCC)CCCCC)CCCN/C(N)=N/[N+](=O)[O-]] |
| I 275 | 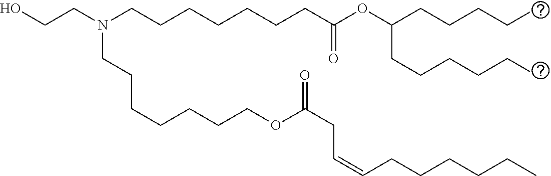 [SMILES: CCCCCC/C=C\CC(=O)OCCCCCCCN(CCO)CCCCCCCC(=O)OC(CCCCC)CCCCCC] |
| I 276 | 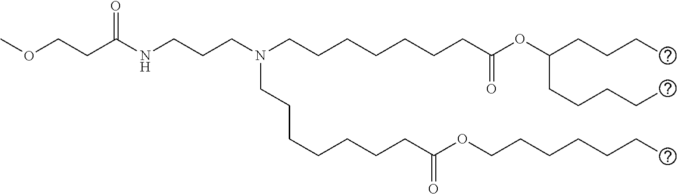 [SMILES: CCCCCCCOC(=O)CCCCCCCN(CCCCCCCC(=O)OC(CCCC)CCCCC)CCCNC(=O)CCOC] |
| I 277 | 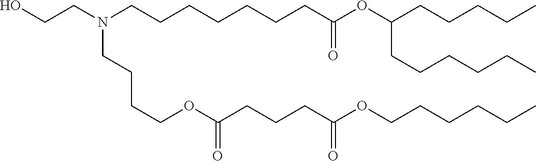 [SMILES: CCCCCCOC(=O)CCCC(=O)OCCCCN(CCO)CCCCCCCC(=O)OC(CCCCC)CCCCCC] |
| I 278 | 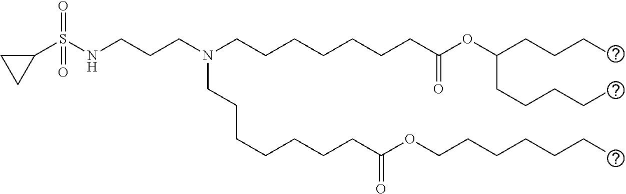 [SMILES: CCCCCCCOC(=O)CCCCCCCN(CCCCCCCC(=O)OC(CCCC)CCCCC)CCCNS(=O)(=O)C1CC1] |
| I 279 | 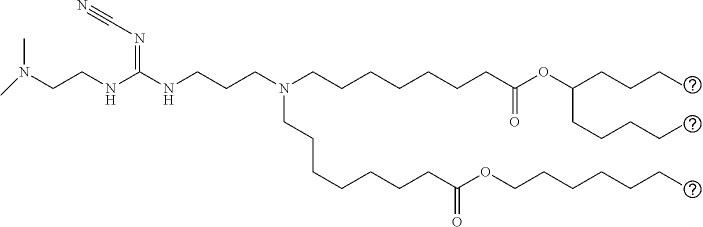 [SMILES: CCCCCCCOC(=O)CCCCCCCN(CCCCCCCC(=O)OC(CCCC)CCCCC)CCCN/C(=N/C#N)NCCN(C)C] |
| I 280 | 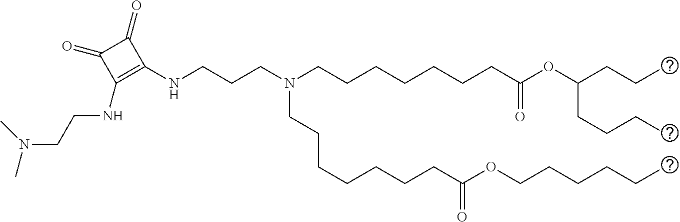 [SMILES: CCCCCCOC(=O)CCCCCCCN(CCCCCCCC(=O)OC(CCC)CCCC)CCCNc1c(NCCN(C)C)c(=O)c1=O] |
| I 281 | 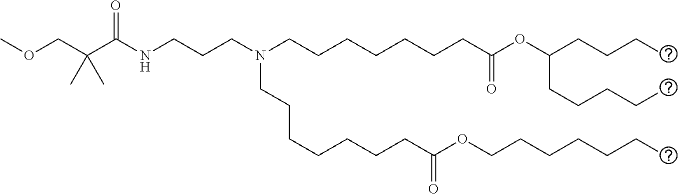 [SMILES: CCCCCCCOC(=O)CCCCCCCN(CCCCCCCC(=O)OC(CCCC)CCCCC)CCCNC(=O)C(C)(C)COC] |
| I 282 | 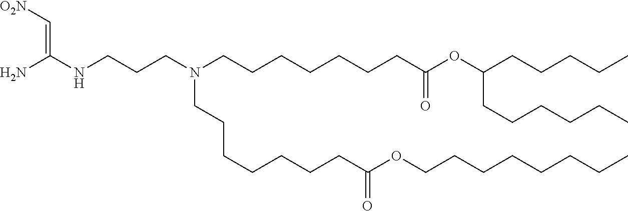 [SMILES: CCCCCCCCOC(=O)CCCCCCCN(CCCCCCCC(=O)OC(CCCCC)CCCCCC)CCCN/C(N)=C/[N+](=O)[O-]] |
| I 283 | 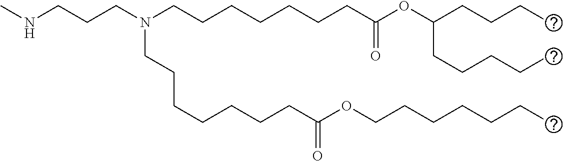 [SMILES: CCCCCCCOC(=O)CCCCCCCN(CCCCCCCC(=O)OC(CCCC)CCCCC)CCCNC] |
| I 284 | 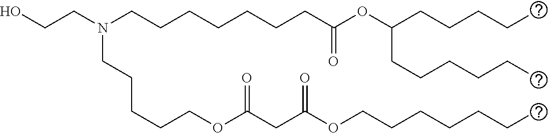 [SMILES: CCCCCCCOC(=O)CC(=O)OCCCCCN(CCO)CCCCCCCC(=O)OC(CCCCC)CCCCCC] |
| I 285 | 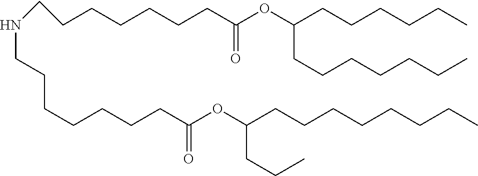 [SMILES: CCCCCCCCC(CCC)OC(=O)CCCCCCCNCCCCCCCC(=O)OC(CCCCCC)CCCCCCC] |
| I 286 | 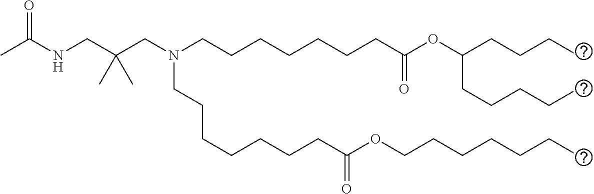 [SMILES: CCCCCCCOC(=O)CCCCCCCN(CCCCCCCC(=O)OC(CCCC)CCCCC)CC(C)(C)CNC(C)=O] |
| I 287 | 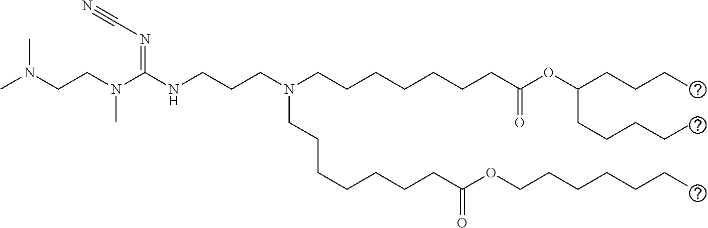 [SMILES: CCCCCCCOC(=O)CCCCCCCN(CCCCCCCC(=O)OC(CCCC)CCCCC)CCCN/C(=N/C#N)N(C)CCN(C)C] |
| I 288 | 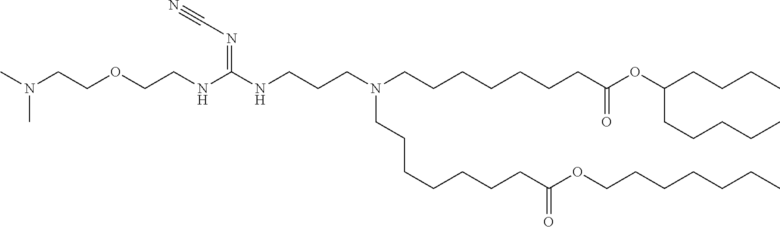 [SMILES: CCCCCCCOC(=O)CCCCCCCN(CCCCCCCC(=O)OC(CCCC)CCCCC)CCCN/C(=N/C#N)NCCOCCN(C)C] |
| I 289 | 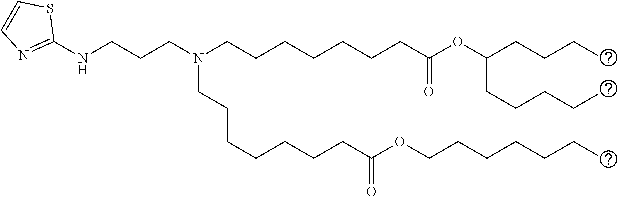 [SMILES: CCCCCCCOC(=O)CCCCCCCN(CCCCCCCC(=O)OC(CCCC)CCCCC)CCCNc1nccs1] |
| I 290 | 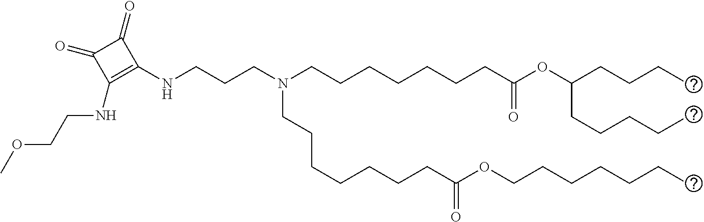 [SMILES: CCCCCCCOC(=O)CCCCCCCN(CCCCCCCC(=O)OC(CCCC)CCCCC)CCCNc1c(NCCOC)c(=O)c1=O] |
| I 291 | 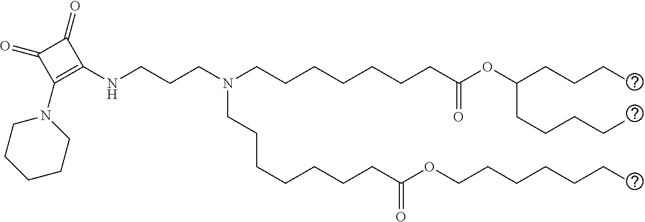 [SMILES: CCCCCCCOC(=O)CCCCCCCN(CCCCCCCC(=O)OC(CCCC)CCCCC)CCCNc1c(N2CCCCC2)c(=O)c1=O] |
| I 292 | 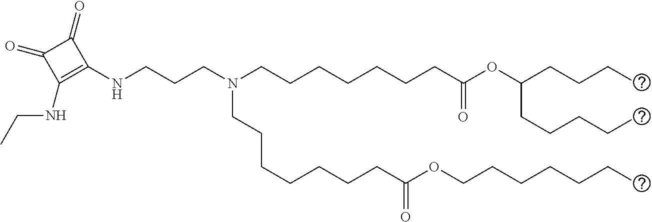 [SMILES: CCCCCCCOC(=O)CCCCCCCN(CCCCCCCC(=O)OC(CCCC)CCCCC)CCCNc1c(NCC)c(=O)c1=O] |
| I 293 | 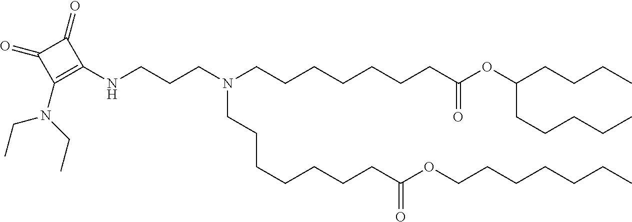 [SMILES: CCCCCCCOC(=O)CCCCCCCN(CCCCCCCC(=O)OC(CCCC)CCCCC)CCCNc1c(N(CC)CC)c(=O)c1=O] |
| I 294 | 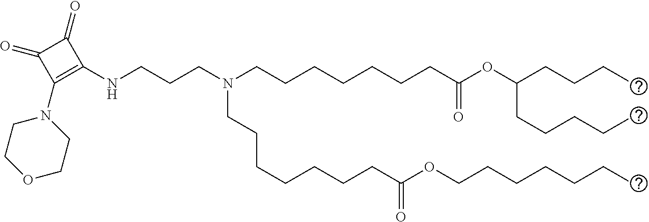 [SMILES: CCCCCCCOC(=O)CCCCCCCN(CCCCCCCC(=O)OC(CCCC)CCCCC)CCCNc1c(N2CCOCC2)c(=O)c1=O] |
| I 295 | 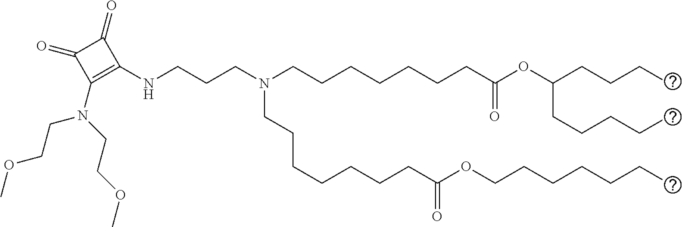 [SMILES: CCCCCCCOC(=O)CCCCCCCN(CCCCCCCC(=O)OC(CCCC)CCCCC)CCCNc1c(N(CCOC)CCOC)c(=O)c1=O] |
| I 296 | 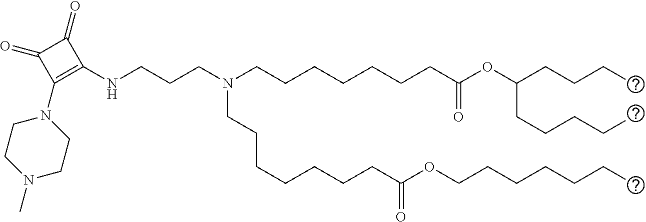 [SMILES: CCCCCCCOC(=O)CCCCCCCN(CCCCCCCC(=O)OC(CCCC)CCCCC)CCCNc1c(N2CCN(C)CC2)c(=O)c1=O] |
| I 297 | 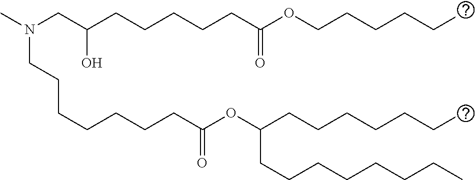 [SMILES: CCCCCCCCC(CCCCCCC)OC(=O)CCCCCCCN(C)CC(O)CCCCCC(=O)OCCCCCC] |
| I 298 | 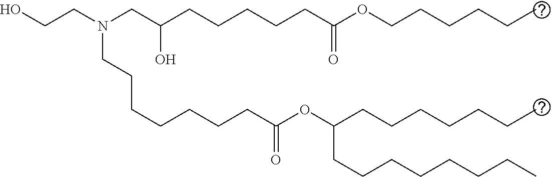 [SMILES: CCCCCCCCC(CCCCCCC)OC(=O)CCCCCCCN(CCO)CC(O)CCCCCC(=O)OCCCCCC] |
| I 299 | 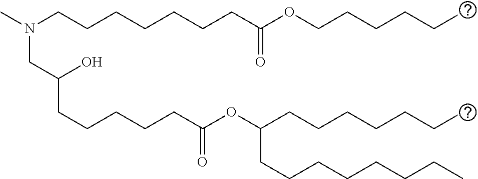 [SMILES: CCCCCCCCC(CCCCCCC)OC(=O)CCCCCC(O)CN(C)CCCCCCCC(=O)OCCCCCC] |
| I 300 | 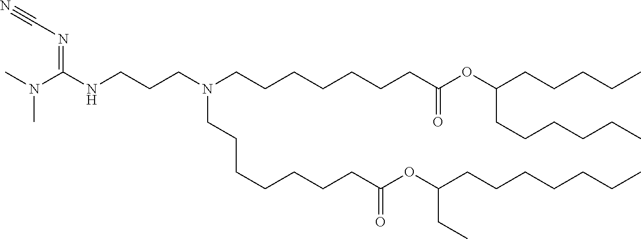 [SMILES: CCCCCCCC(CC)OC(=O)CCCCCCCN(CCCCCCCC(=O)OC(CCCCC)CCCCCC)CCCN/C(=N/C#N)N(C)C] |
| I 301 | 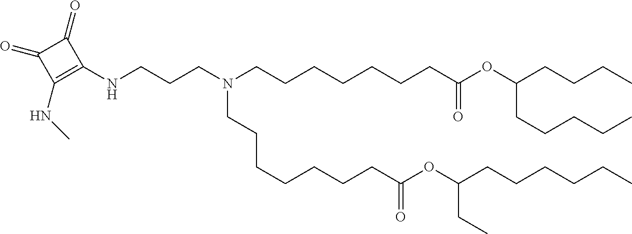 [SMILES: CCCCCCC(CC)OC(=O)CCCCCCCN(CCCCCCCC(=O)OC(CCCC)CCCCC)CCCNc1c(NC)c(=O)c1=O] |
| I 302 | 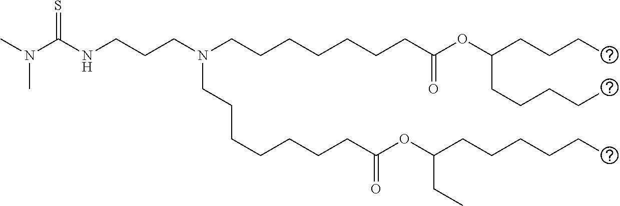 [SMILES: CCCCCCC(CC)OC(=O)CCCCCCCN(CCCCCCCC(=O)OC(CCCC)CCCCC)CCCNC(=S)N(C)C] |
| I 303 | 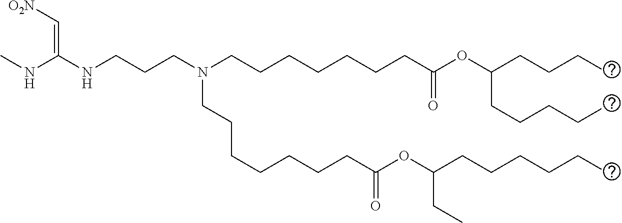 [SMILES: CCCCCCC(CC)OC(=O)CCCCCCCN(CCCCCCCC(=O)OC(CCCC)CCCCC)CCCN/C(=C/[N+](=O)[O-])NC] |
| I 304 | 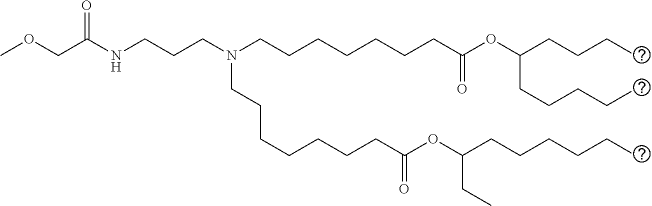 [SMILES: CCCCCCC(CC)OC(=O)CCCCCCCN(CCCCCCCC(=O)OC(CCCC)CCCCC)CCCNC(=O)COC] |
| I 305 | 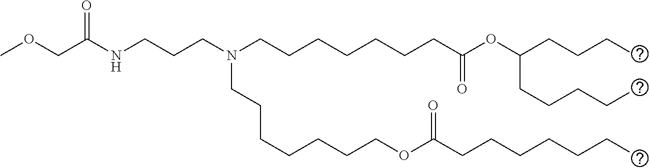 [SMILES: CCCCCCCC(=O)OCCCCCCCN(CCCCCCCC(=O)OC(CCCC)CCCCC)CCCNC(=O)COC] |
| I 306 | 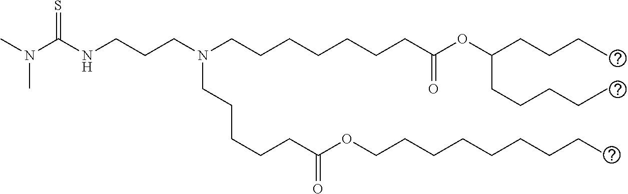 [SMILES: CCCCCCCCCOC(=O)CCCCCN(CCCCCCCC(=O)OC(CCCC)CCCCC)CCCNC(=S)N(C)C] |
| I 307 | 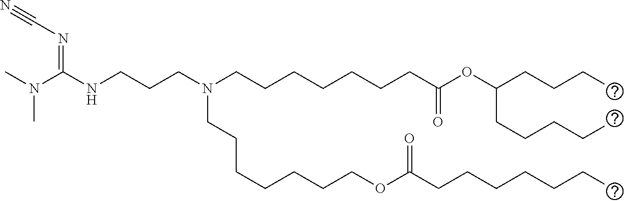 [SMILES: CCCCCCCC(=O)OCCCCCCCN(CCCCCCCC(=O)OC(CCCC)CCCCC)CCCN/C(=N/C#N)N(C)C] |
| I 308 | 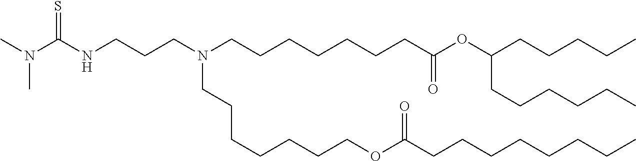 [SMILES: CCCCCCCCC(=O)OCCCCCCCN(CCCCCCCC(=O)OC(CCCCC)CCCCCC)CCCNC(=S)N(C)C] |
| I 309 | 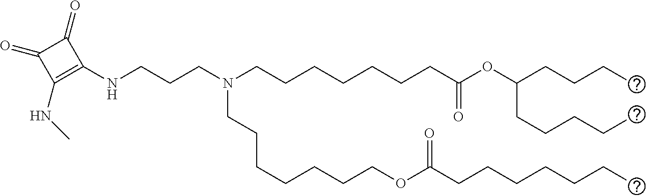 [SMILES: CCCCCCCC(=O)OCCCCCCCN(CCCCCCCC(=O)OC(CCCC)CCCCC)CCCNc1c(NC)c(=O)c1=O] |
| I 310 | 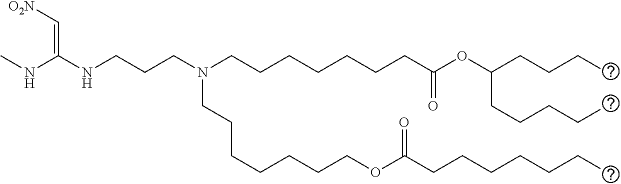 [SMILES: CCCCCCCC(=O)OCCCCCCCN(CCCCCCCC(=O)OC(CCCC)CCCCC)CCCN/C(=C/[N+](=O)[O-])NC] |
| I 311 | 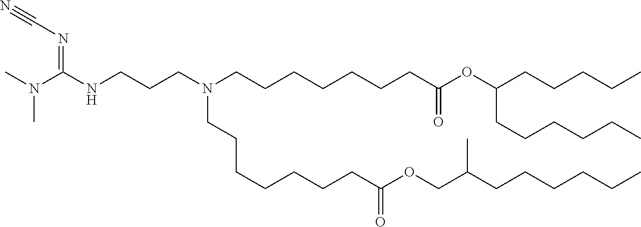 [SMILES: CCCCCCC(C)COC(=O)CCCCCCCN(CCCCCCCC(=O)OC(CCCCC)CCCCCC)CCCN/C(=N/C#N)N(C)C] |
| I 312 | 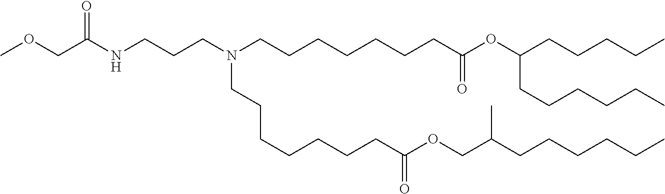 [SMILES: CCCCCCC(C)COC(=O)CCCCCCCN(CCCCCCCC(=O)OC(CCCCC)CCCCCC)CCCNC(=O)COC] |
| I 313 | 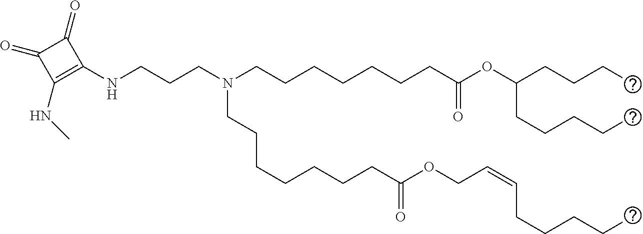 [SMILES: CCCCC/C=C\COC(=O)CCCCCCCN(CCCCCCCC(=O)OC(CCCC)CCCCC)CCCNc1c(NC)c(=O)c1=O] |
| I 314 | 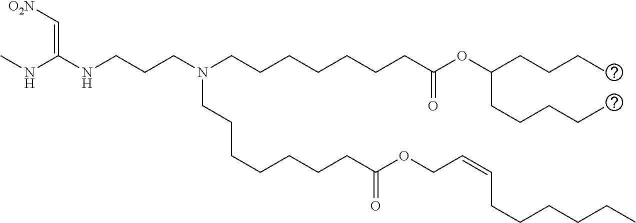 [SMILES: CCCCCC/C=C\COC(=O)CCCCCCCN(CCCCCCCC(=O)OC(CCCC)CCCCC)CCCN/C(=C/[N+](=O)[O-])NC] |
| I 315 | 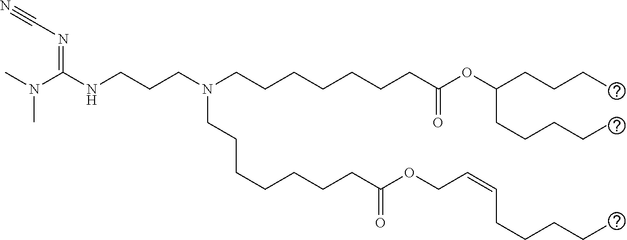 [SMILES: CCCCC/C=C\COC(=O)CCCCCCCN(CCCCCCCC(=O)OC(CCCC)CCCCC)CCCN/C(=N/C#N)N(C)C] |
| I 316 | 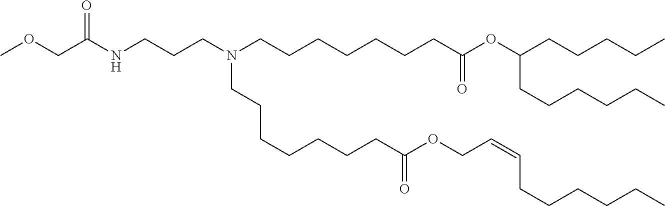 [SMILES: CCCCCC/C=C\COC(=O)CCCCCCCN(CCCCCCCC(=O)OC(CCCCC)CCCCCC)CCCNC(=O)COC] |
| I 317 | 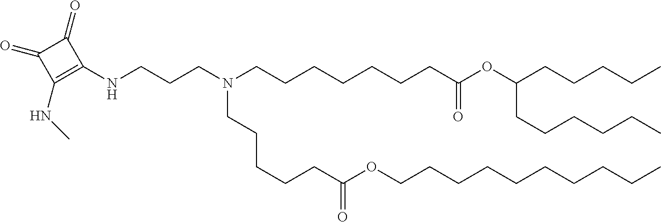 [SMILES: CCCCCCCCCCOC(=O)CCCCCN(CCCCCCCC(=O)OC(CCCCC)CCCCCC)CCCNc1c(NC)c(=O)c1=O] |
| I 318 | 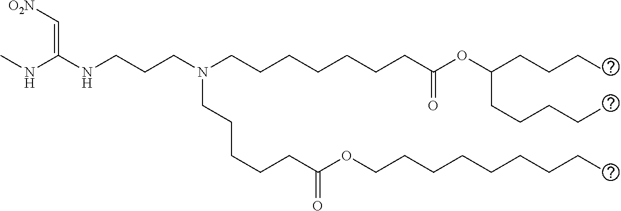 [SMILES: CCCCCCCCCOC(=O)CCCCCN(CCCCCCCC(=O)OC(CCCC)CCCCC)CCCN/C(=C/[N+](=O)[O-])NC] |
| I 319 | 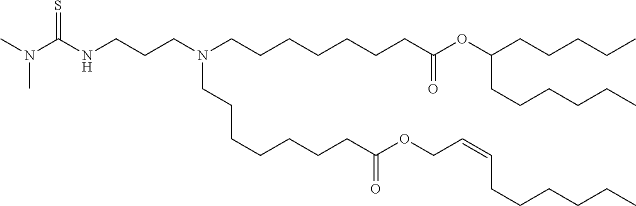 [SMILES: CCCCCC/C=C\COC(=O)CCCCCCCN(CCCCCCCC(=O)OC(CCCCC)CCCCCC)CCCNC(=S)N(C)C] |
| I 320 | 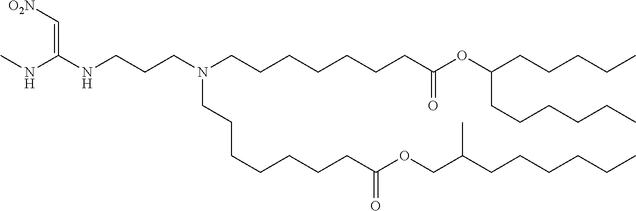 [SMILES: CCCCCCC(C)COC(=O)CCCCCCCN(CCCCCCCC(=O)OC(CCCCC)CCCCCC)CCCN/C(=C/[N+](=O)[O-])NC] |
| I 321 | 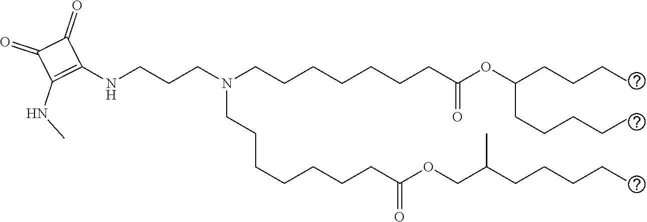 [SMILES: CCCCCC(C)COC(=O)CCCCCCCN(CCCCCCCC(=O)OC(CCCC)CCCCC)CCCNc1c(NC)c(=O)c1=O] |
| I 322 | 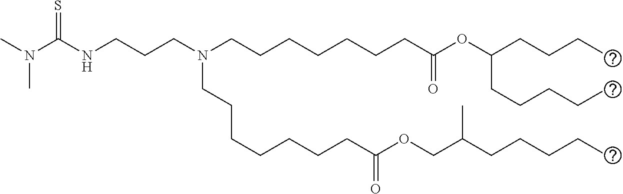 [SMILES: CCCCCC(C)COC(=O)CCCCCCCN(CCCCCCCC(=O)OC(CCCC)CCCCC)CCCNC(=S)N(C)C] |
| I 323 | 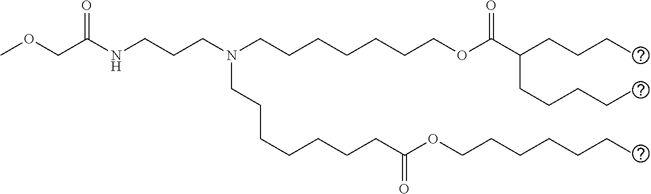 [SMILES: CCCCCCCOC(=O)CCCCCCCN(CCCCCCCOC(=O)C(CCCC)CCCCC)CCCNC(=O)COC] |
| I 324 | 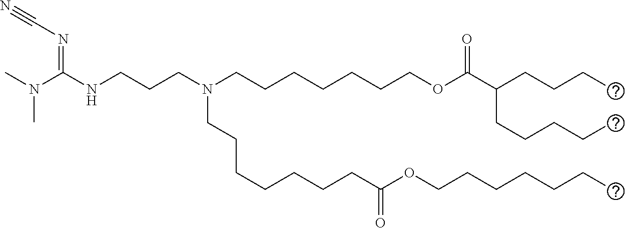 [SMILES: CCCCCCCOC(=O)CCCCCCCN(CCCCCCCOC(=O)C(CCCC)CCCCC)CCCN/C(=N/C#N)N(C)C] |
| I 325 | 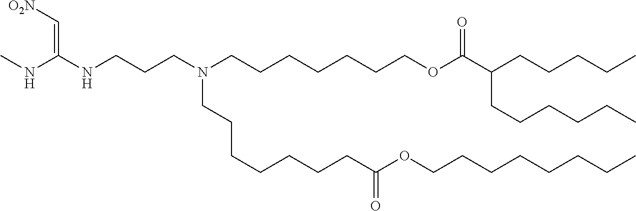 [SMILES: CCCCCCCCOC(=O)CCCCCCCN(CCCCCCCOC(=O)C(CCCCC)CCCCCC)CCCN/C(=C/[N+](=O)[O-])NC] |
| I 326 | 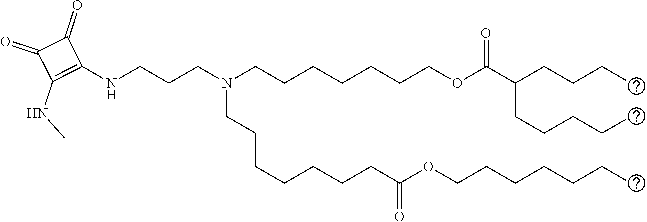 [SMILES: CCCCCCCOC(=O)CCCCCCCN(CCCCCCCOC(=O)C(CCCC)CCCCC)CCCNc1c(NC)c(=O)c1=O] |
| I 327 | 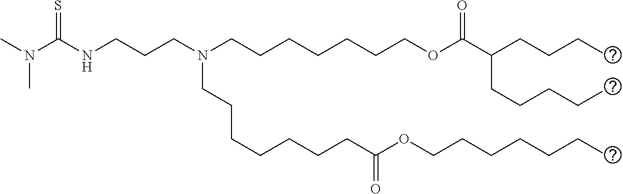 [SMILES: CCCCCCCOC(=O)CCCCCCCN(CCCCCCCOC(=O)C(CCCC)CCCCC)CCCNC(=S)N(C)C] |
| I 328 | 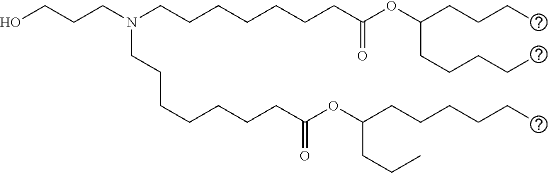 [SMILES: CCCCCCC(CCC)OC(=O)CCCCCCCN(CCCO)CCCCCCCC(=O)OC(CCCC)CCCCC] |
| I 329 | 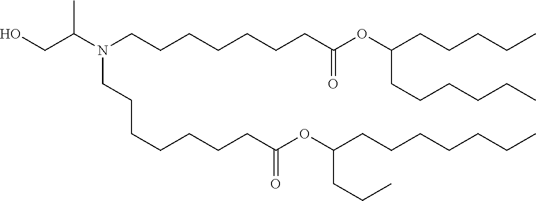 [SMILES: CCCCCCCC(CCC)OC(=O)CCCCCCCN(CCCCCCCC(=O)OC(CCCCC)CCCCCC)C(C)CO] |
| I 330 | 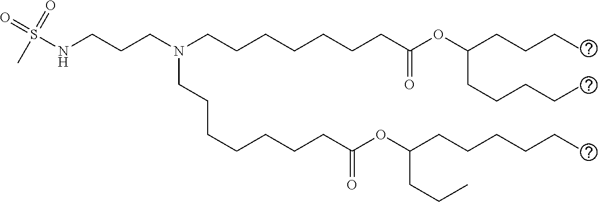 [SMILES: CCCCCCC(CCC)OC(=O)CCCCCCCN(CCCCCCCC(=O)OC(CCCC)CCCCC)CCCNS(C)(=O)=O] |
| I 331 | 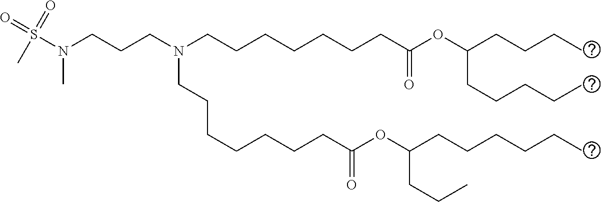 [SMILES: CCCCCCC(CCC)OC(=O)CCCCCCCN(CCCCCCCC(=O)OC(CCCC)CCCCC)CCCN(C)S(C)(=O)=O] |
| I 332 | 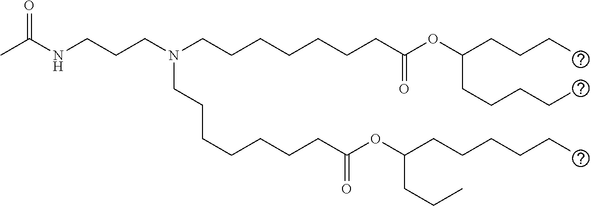 [SMILES: CCCCCCC(CCC)OC(=O)CCCCCCCN(CCCCCCCC(=O)OC(CCCC)CCCCC)CCCNC(C)=O] |
| I 333 | 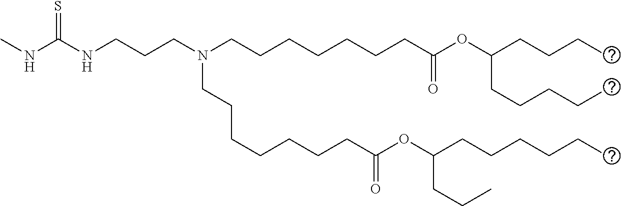 [SMILES: CCCCCCC(CCC)OC(=O)CCCCCCCN(CCCCCCCC(=O)OC(CCCC)CCCCC)CCCNC(=S)NC] |
| I 334 | 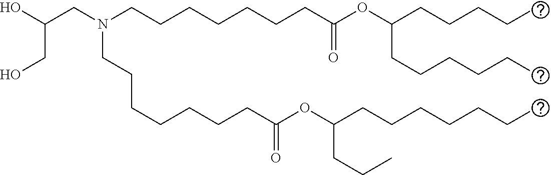 [SMILES: CCCCCCCC(CCC)OC(=O)CCCCCCCN(CCCCCCCC(=O)OC(CCCCC)CCCCCC)CC(O)CO] |
| I 335 | 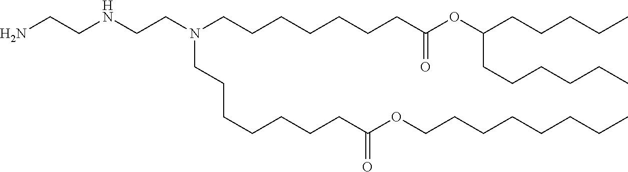 [SMILES: CCCCCCCCOC(=O)CCCCCCCN(CCCCCCCC(=O)OC(CCCCC)CCCCCC)CCNCCN] |
| I 336 | 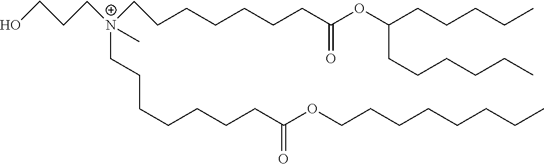 [SMILES: CCCCCCCCOC(=O)CCCCCCC[N+](C)(CCCO)CCCCCCCC(=O)OC(CCCCC)CCCCCC] |
| I 337 | 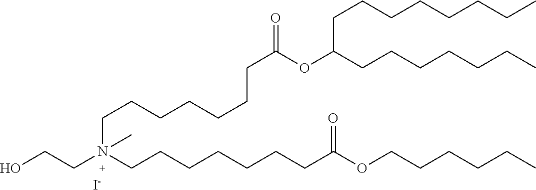 |
| I 338 | 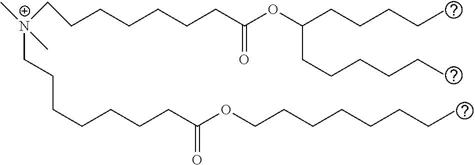 [SMILES: CCCCCCCCOC(=O)CCCCCCC[N+](C)(C)CCCCCCCC(=O)OC(CCCCC)CCCCCC] |
| I 339 | 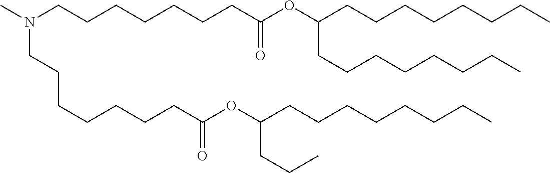 [SMILES: CCCCCCCCC(CCC)OC(=O)CCCCCCCN(C)CCCCCCCC(=O)OC(CCCCCCCC)CCCCCCCC] |
| I 340 | 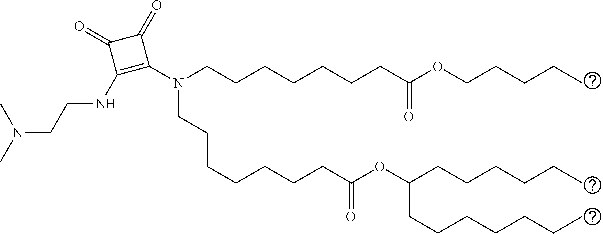 [SMILES: CCCCCCCC(CCCCCC)OC(=O)CCCCCCCN(CCCCCCCC(=O)OCCCCC)c1c(NCCN(C)C)c(=O)c1=O] |
| I 341 | 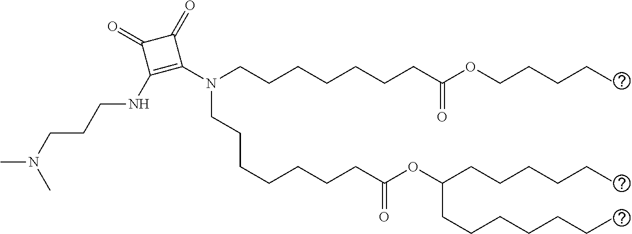 [SMILES: CCCCCCCC(CCCCCC)OC(=O)CCCCCCCN(CCCCCCCC(=O)OCCCCC)c1c(NCCCN(C)C)c(=O)c1=O] |
| I 342 | 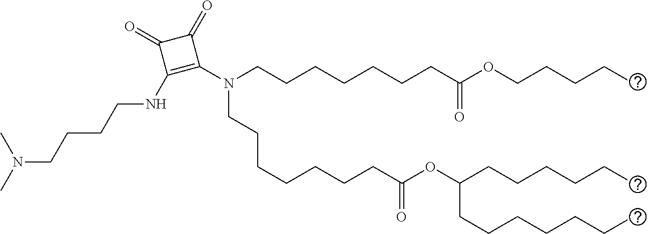 [SMILES: CCCCCCCC(CCCCCC)OC(=O)CCCCCCCN(CCCCCCCC(=O)OCCCCC)c1c(NCCCCN(C)C)c(=O)c1=O] |
| I 343 | 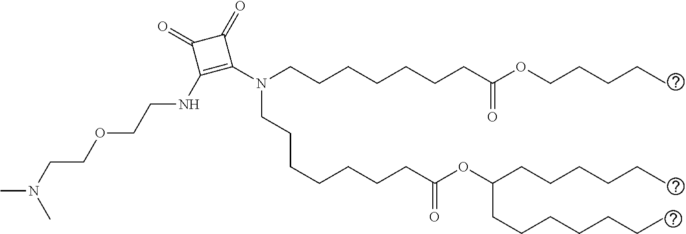 [SMILES: CCCCCCCC(CCCCCC)OC(=O)CCCCCCCN(CCCCCCCC(=O)OCCCCC)c1c(NCCOCCN(C)C)c(=O)c1=O] |
| I 344 | 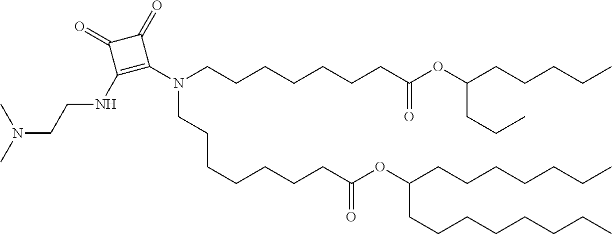 [SMILES: CCCCCCCCC(CCCCCCC)OC(=O)CCCCCCCN(CCCCCCCC(=O)OC(CCC)CCCCC)c1c(NCCN(C)C)c(=O)c1=O] |
| I 345 | 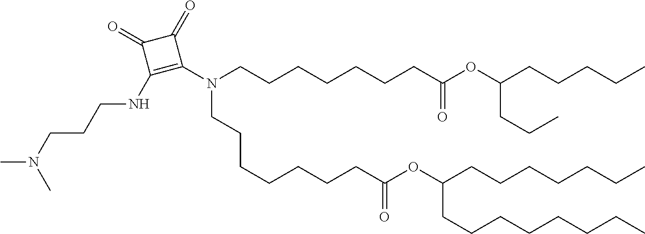 [SMILES: CCCCCCCCC(CCCCCCC)OC(=O)CCCCCCCN(CCCCCCCC(=O)OC(CCC)CCCCC)c1c(NCCCN(C)C)c(=O)c1=O] |
| I 346 | 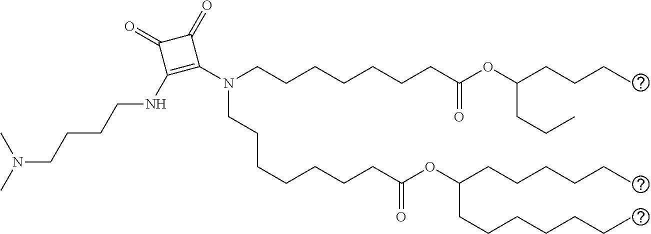 [SMILES: CCCCCCCC(CCCCCC)OC(=O)CCCCCCCN(CCCCCCCC(=O)OC(CCC)CCCC)c1c(NCCCCN(C)C)c(=O)c1=O] |
| I 347 | 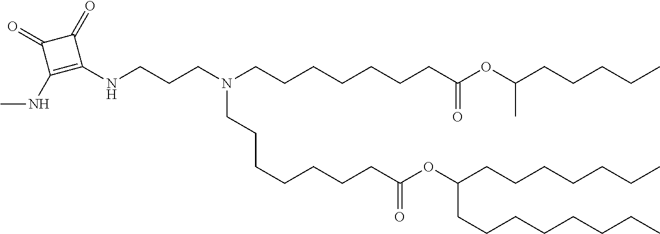 [SMILES: CCCCCCCCC(CCCCCCC)OC(=O)CCCCCCCN(CCCCCCCC(=O)OC(C)CCCCC)CCCNc1c(NC)c(=O)c1=O] |
| I 348 | 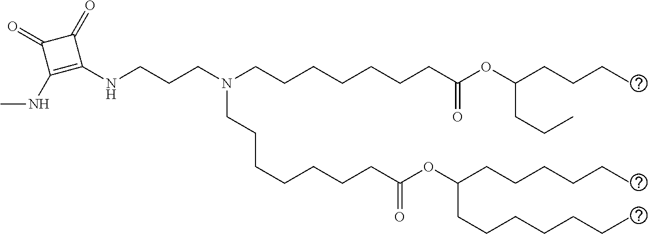 [SMILES: CCCCCCCC(CCCCCC)OC(=O)CCCCCCCN(CCCCCCCC(=O)OC(CCC)CCCC)CCCNc1c(NC)c(=O)c1=O] |
| I 349 | 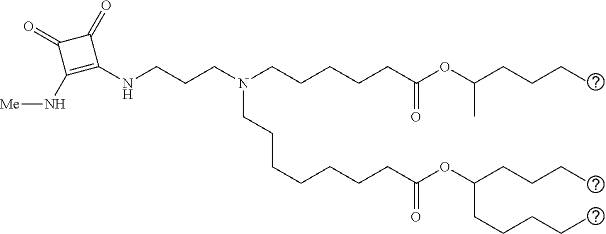 [SMILES: CCCCCC(CCCC)OC(=O)CCCCCCCN(CCCCCC(=O)OC(C)CCCC)CCCNc1c(NC)c(=O)c1=O] |
| I 350 | 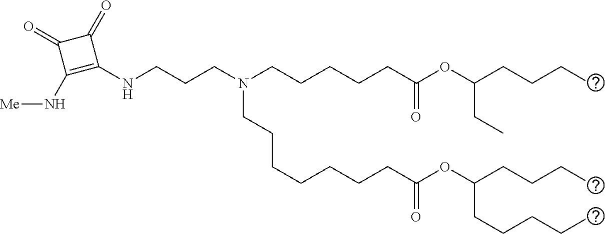 [SMILES: CCCCCC(CCCC)OC(=O)CCCCCCCN(CCCCCC(=O)OC(CC)CCCC)CCCNc1c(NC)c(=O)c1=O] |
| I 351 | 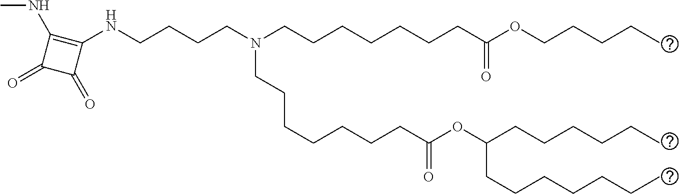 [SMILES: CCCCCCCC(CCCCCC)OC(=O)CCCCCCCN(CCCCCCCC(=O)OCCCCC)CCCCNc1c(NC)c(=O)c1=O] |
| I 352 | 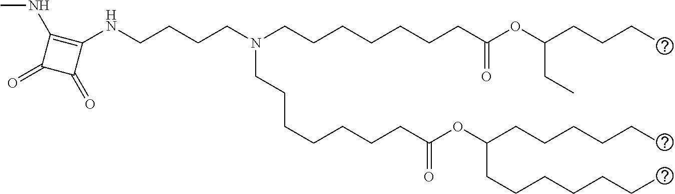 [SMILES: CCCCCCCC(CCCCCC)OC(=O)CCCCCCCN(CCCCCCCC(=O)OC(CC)CCCC)CCCCNc1c(NC)c(=O)c1=O] |
| I 353 | 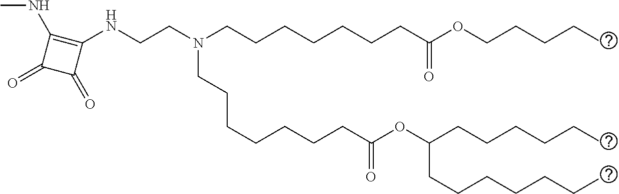 [SMILES: CCCCCCCC(CCCCCC)OC(=O)CCCCCCCN(CCCCCCCC(=O)OCCCCC)CCNc1c(NC)c(=O)c1=O] |
| I 354 | 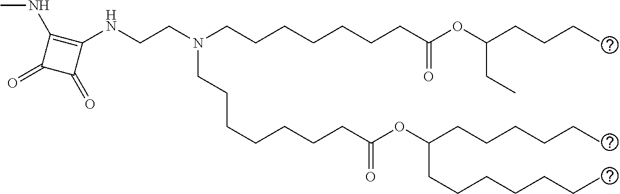 [SMILES: CCCCCCCC(CCCCCC)OC(=O)CCCCCCCN(CCCCCCCC(=O)OC(CC)CCCC)CCNc1c(NC)c(=O)c1=O] |
| I 355 | 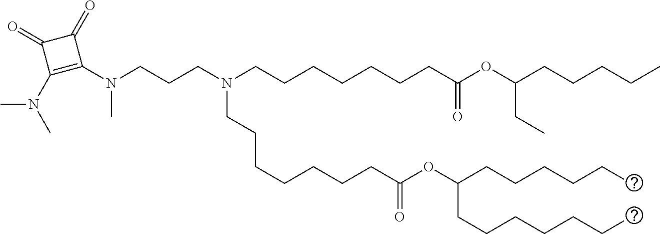 [SMILES: CCCCCCCC(CCCCCC)OC(=O)CCCCCCCN(CCCCCCCC(=O)OC(CC)CCCCC)CCCN(C)c1c(N(C)C)c(=O)c1=O] |

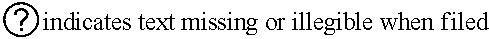

In some embodiments, a lipid of the disclosure comprises Compound I-340A:

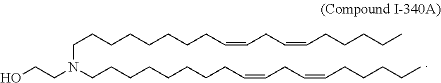 [SMILES: CCCCC/C=C\C/C=C\CCCCCCCCN(CCO)CCCCCCCC/C=C\C/C=C\CCCCC]

The central amine moiety of a lipid according to Formula (I I), (I IA), I (IB), I (II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), or (I VIIId) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some aspects, the ionizable lipids of the present disclosure may be one or more of compounds of formula I (I IX),

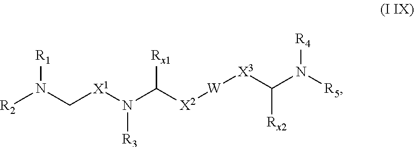

or salts or isomers thereof, wherein

W is

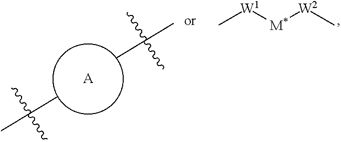

ring A is

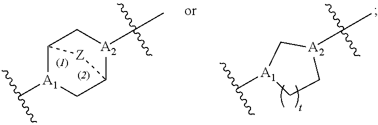

t is 1 or 2;

A₁ and A₂ are each independently selected from CH or N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;

R_{X1} and R_{X2} are each independently H or C₁₋₃ alkyl;

each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;

M* is C₁-C₆ alkyl,

W¹ and W² are each independently selected from the group consisting of —O— and —N(R₆)—;

each R₆ is independently selected from the group consisting of H and C₁₋₅ alkyl;

X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H;

each R″ is independently selected from the group consisting of C₃₋₁₂ alkyl, C₃₋₁₂ alkenyl and —R*MR′; and

n is an integer from 1-6;

wherein when ring A is

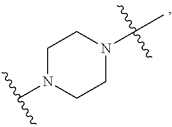 [SMILES: *N1CCN(*)CC1]

then

i) at least one of X¹, X², and X³ is not —CH₂—; and/or

ii) at least one of R₁, R₂, R₃, R₄, and R₅ is —R″MR′.

In some embodiments, the compound is of any of formulae (I IXa1)-(I IXa8):

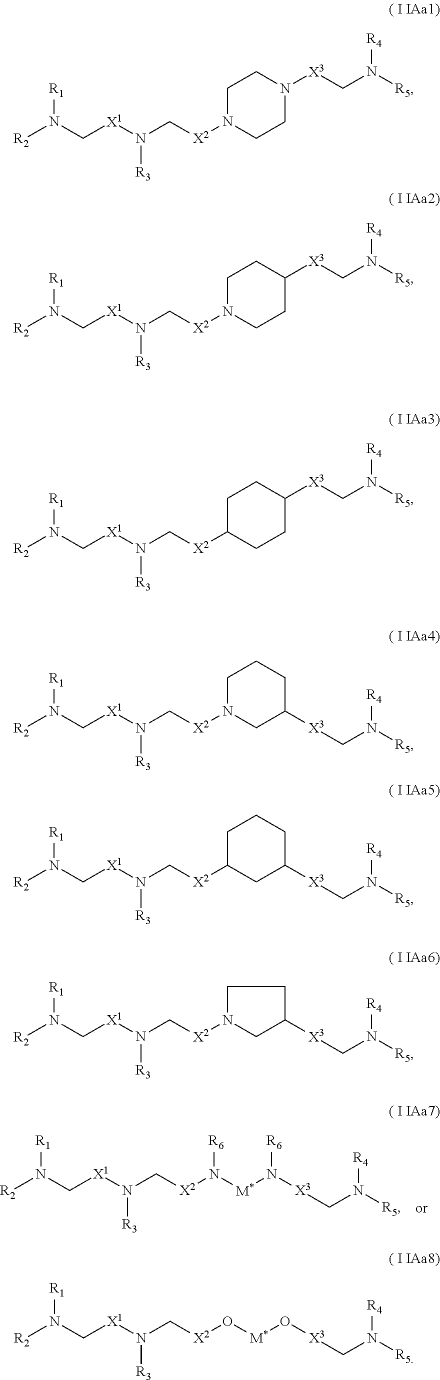

In some embodiments, the ionizable lipids are one or more of the compounds described in U.S. Application Nos. 62/271,146, 62/338,474, 62/413,345, and 62/519,826, and PCT Application No. PCT/US2016/068300.

In some embodiments, the ionizable lipids are selected from Compounds 1-156 described in U.S. Application No. 62/519,826.

In some embodiments, the ionizable lipids are selected from Compounds 1-16, 42-66, 68-76, and 78-156 described in U.S. Application No. 62/519,826.

In some embodiments, the ionizable lipid is

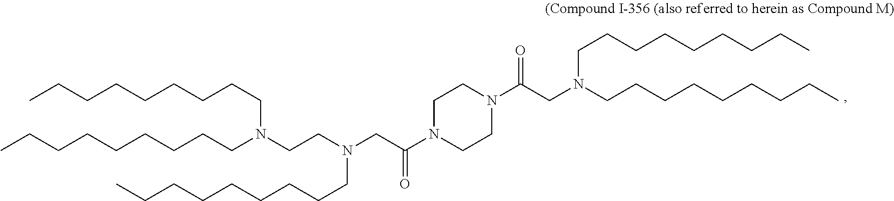 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

In some embodiments, the ionizable lipid is

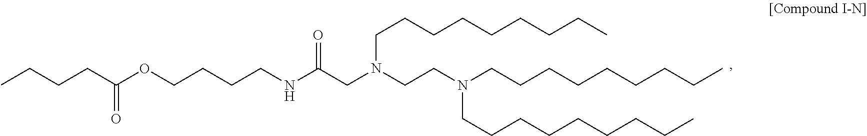 [SMILES: CCCCCCCCCN(CCCCCCCCC)CCN(CCCCCCCCC)CC(=O)NCCCCOC(=O)CCCC]

or a salt thereof.

In some embodiments, the ionizable lipid is

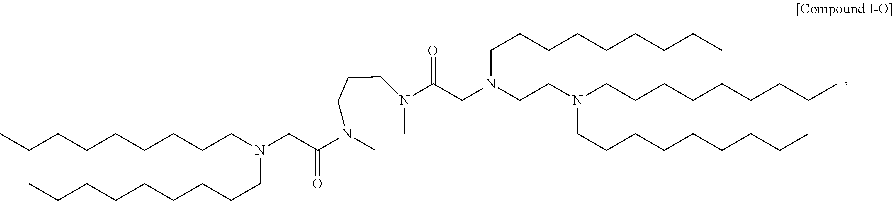 [SMILES: CCCCCCCCCN(CCCCCCCCC)CCN(CCCCCCCCC)CC(=O)N(C)CCCN(C)C(=O)CN(CCCCCCCCC)CCCCCCCCC]

or a salt thereof.

In some embodiments, the ionizable lipid is

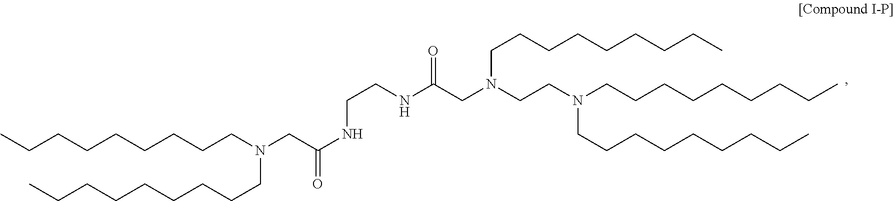 [SMILES: CCCCCCCCCN(CCCCCCCCC)CCN(CCCCCCCCC)CC(=O)NCCNC(=O)CN(CCCCCCCCC)CCCCCCCCC]

or a salt thereof.

In some embodiments, the ionizable lipid is

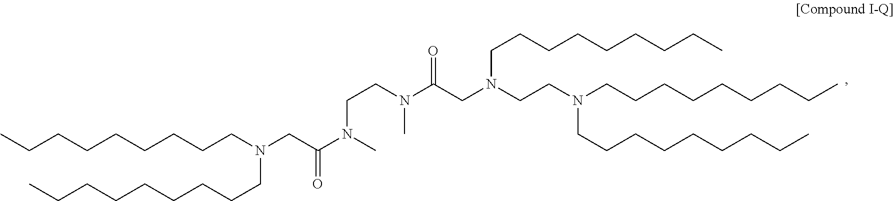 [SMILES: CCCCCCCCCN(CCCCCCCCC)CCN(CCCCCCCCC)CC(=O)N(C)CCN(C)C(=O)CN(CCCCCCCCC)CCCCCCCCC]

or a salt thereof.

The central amine moiety of a lipid according to any of the Formulae herein, e.g. a compound having any of Formula (I I), (I IA), (IIB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some embodiments, the amount the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (Vila), (Villa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5). (IXa6), (IXa7), or (IXa8)) (each of these preceded by the letter I for clarity) ranges from about 1 mol % to 99 mol % in the lipid composition.

In one embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mol % in the lipid composition.

In one embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (aa5). (IXa6), (IXa7), or (IXa8) (each of these preceded by the letter I for clarity) ranges from about 30 mol % to about 70 mol %, from about 35 mol % to about 65 mol %, from about 40 mol % to about 60 mol %, and from about 45 mol % to about 55 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (aa5). (IXa6), (IXa7), or (IXa8) (each of these preceded by the letter I for clarity) is about 45 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5). (IXa6), (IXa7), or (IXa8) (each of these preceded by the letter I for clarity) is about 40 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5). (IXa6), (IXa7), or (IXa8) (each of these preceded by the letter I for clarity) is about 50 mol % in the lipid composition.

In addition to the ionizable amino lipid disclosed herein, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8), (each of these preceded by the letter I for clarity) the lipid-based composition (e.g., lipid nanoparticle) disclosed herein can comprise additional components such as cholesterol and/or cholesterol analogs, non-cationic helper lipids, structural lipids, PEG-lipids, and any combination thereof.

Additional ionizable lipids of the invention can be selected from the non-limiting group consisting of 3-(didodecylamino)—N1,N1,4-tridodecyl-1-piperazineethanamine (KL10),
N1-[2-(didodecylamino)ethyl]-N1,N4,N4-tridodecyl-1,4-piperazinediethanamine (KL22),
14,25-ditridecyl-15,18,21,24-tetraaza-octatriacontane (KL25),
1,2-dilinoleyloxy-N,N-dimethylaminopropane (DLin-DMA),
2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA),
2,2-dilinoleyl-4-(2-dimethylaminoethyl)-[1,3]-dioxolane (DLin-KC2-DMA),
1,2-dioleyloxy-N,N-dimethylaminopropane (DODMA), (13Z,165Z)—N,N-dimethyl-3-nonydocosa-13-16-dien-1-amine (L608),
2-({8-[(3β)-cholest-5-en-3-yloxy]octyl}oxy)-N,N-dimethyl-3-[(9Z,12Z)-octadeca-9,12-dien-1-yl oxy]propan-1-amine (Octyl-CLinDMA),
(2R)-2-({8-[(3β)-cholest-5-en-3-yloxy]octyl}oxy)-N,N-dimethyl-3-[(9Z,12Z)-octadeca-9,12-die n-1-yloxy]propan-1-amine (Octyl-CLinDMA (2R)), and
(2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid can also be a lipid including a cyclic amine group.

Ionizable lipids of the invention can also be the compounds disclosed in International Publication No. WO 2017/075531 A₁, hereby incorporated by reference in its entirety. For example, the ionizable amino lipids include, but not limited to:

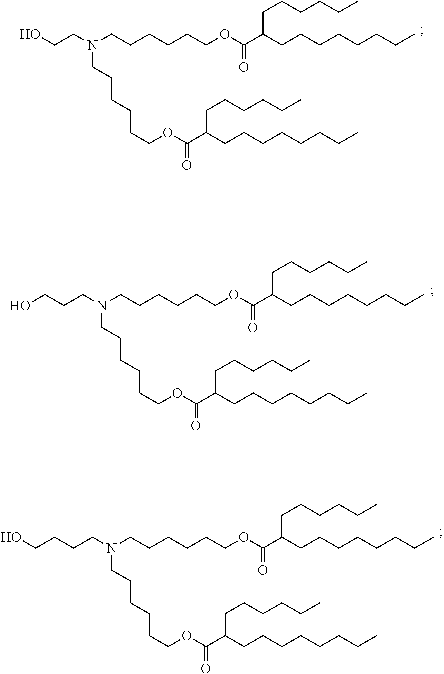 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC]

and any combination thereof.

Ionizable lipids of the invention can also be the compounds disclosed in International Publication No. WO 2015/199952 A₁, hereby incorporated by reference in its entirety. For example, the ionizable amino lipids include, but not limited to:

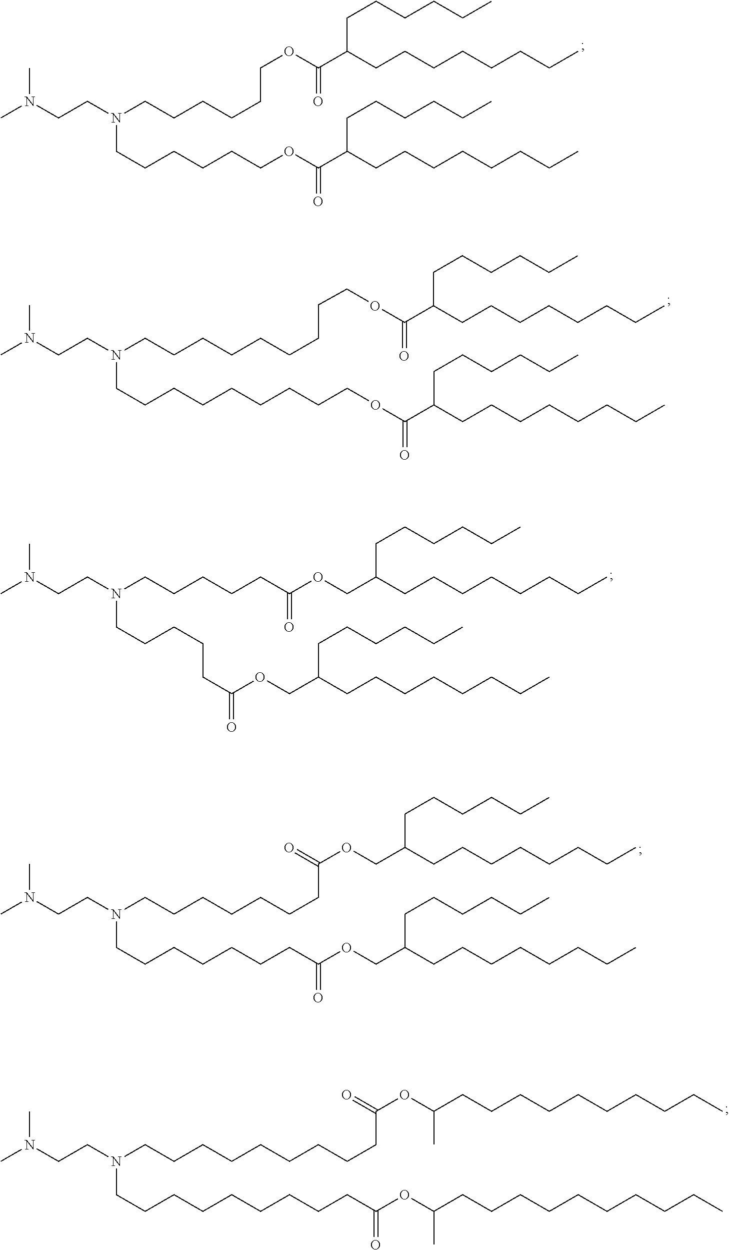

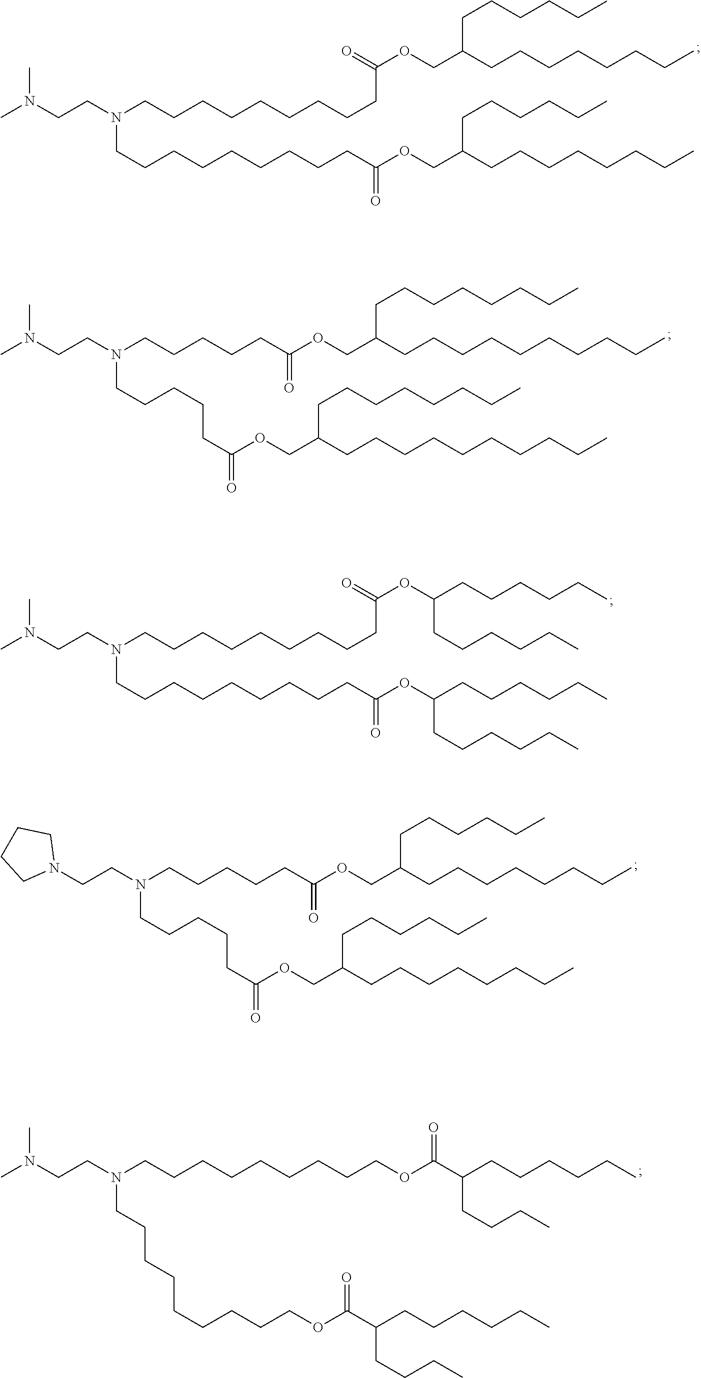

and any combination thereof.

In any of the foregoing or related aspects, the ionizable lipid of the LNP of the disclosure comprises a compound included in any e.g. a compound having any of Formula (I), (IA), (IB), (II), (IIa), (IIb), (IIc), (IId), (IIe), MD, (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity).

In any of the foregoing or related aspects, the ionizable lipid of the LNP of the disclosure comprises a compound comprising any of Compound Nos. I 1-356.

In any of the foregoing or related aspects, the ionizable lipid of the LNP of the disclosure comprises at least one compound selected from the group consisting of: Compound Nos. I 18 (also referred to as Compound X), I 25 (also referred to as Compound Y), I 48, I 50, I 109, I 111, I 113, I 181, I 182, I 244, I 292, I 301, I 321, I 322, I 326, I 328, I 330, I 331, and I 332. In another embodiment, the ionizable lipid of the LNP of the disclosure comprises a compound selected from the group consisting of: Compound Nos. I 18 (also referred to as Compound X), I 25 (also referred to as Compound Y), I 48, I 50, I 109, I 111, I 181, I 182, I 292, I 301, I 321, I 326, I 328, and I 330. In another embodiment, the ionizable lipid of the LNP of the disclosure comprises a compound selected from the group consisting of: Compound Nos. I 182, I 301, I 321, and I 326.

In any of the foregoing or related aspects, the synthesis of compounds of the invention, e.g. compounds comprising any of Compound Nos. 1-356, follows the synthetic descriptions in U.S. Provisional Patent Application No. 62/733,315, filed Sep. 19, 2018.

Representative Synthetic Routes:

### Compound I-182: Heptadecan-9-yl 8-((3-((2-(methylamino)-3,4-dioxocyclobut-1-en-1-yl)amino)propyl)(8-(nonyloxy)-8-oxooctyl)amino)octanoate 3-Methoxy-4-(methylamino)cyclobut-3-ene-1,2-dione

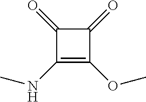 [0881] Chemical Formula: C₆H₇NO₃
Molecular Weight: 141.13

To a solution of 3,4-dimethoxy-3-cyclobutene-1,2-dione (1 g, 7 mmol) in 100 mL diethyl ether was added a 2M methylamine solution in THF (3.8 mL, 7.6 mmol) and a ppt. formed almost immediately. The mixture was stirred at rt for 24 hours, then filtered, the filter solids washed with diethyl ether and air-dried. The filter solids were dissolved in hot EtOAc, filtered, the filtrate allowed to cool to room temp., then cooled to 0° C. to give a ppt. This was isolated via filtration, washed with cold EtOAc, air-dried, then dried under vacuum to give 3-methoxy-4-(methylamino)cyclobut-3-ene-1,2-dione (0.70 g, 5 mmol, 73%) as a white solid. ¹H NMR (300 MHz, DMSO-d₆) δ: ppm 8.50 (br. d, 1H, J=69 Hz); 4.27 (s, 3H); 3.02 (sdd, 3H, J=42 Hz, 4.5 Hz).

### Heptadecan-9-yl 8-((3-((2-(methylamino)-3,4-dioxocyclobut-1-en-1-yl)amino)propyl)(8-(nonyloxy)-8-oxooctyl)amino)octanoate

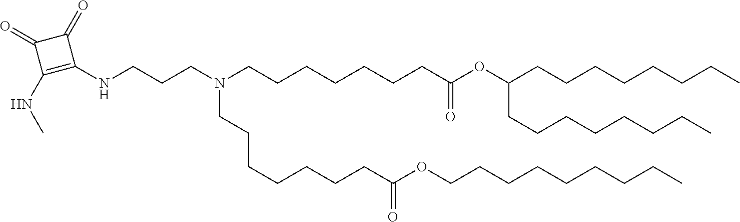 [SMILES: CCCCCCCCCOC(=O)CCCCCCCN(CCCCCCCC(=O)OC(CCCCCCCC)CCCCCCCC)CCCNc1c(NC)c(=O)c1=O] [0885] Chemical Formula: C₅₀H₉₃N₃O₆
Molecular Weight: 832.31

To a solution of heptadecan-9-yl 8-((3-aminopropyl)(8-(nonyloxy)-8-oxooctyl)amino)octanoate (200 mg, 0.28 mmol) in 10 mL ethanol was added 3-methoxy-4-(methylamino)cyclobut-3-ene-1,2-dione (39 mg, 0.28 mmol) and the resulting colorless solution stirred at rt for 20 hours after which no starting amine remained by LC/MS. The solution was concentrated in vacuo and the residue purified by silica gel chromatography (0-100% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-(nonyloxy)-8-oxooctyl)amino)octanoate (138 mg, 0.17 mmol, 60%) as a gummy white solid. UPLC/ELSD: RT=3. min. MS (ES): m/z (MH⁺) 833.4 for C₅₁H₉₅N₃O₆. ¹H NMR (300 MHz, CDCl₃) δ: ppm 7.86 (br. s., 1H); 4.86 (quint., 1H, J=6 Hz); 4.05 (t, 2H, J=6 Hz); 3.92 (d, 2H, J=3 Hz); 3.20 (s, 6H); 2.63 (br. s, 2H); 2.42 (br. s, 3H); 2.28 (m, 4H); 1.74 (br. s, 2H); 1.61 (m, 8H); 1.50 (m, 5H); 1.41 (m, 3H); 1.25 (br. m, 47H); 0.88 (t, 9H, J=7.5 Hz).

### Compound I-301: Heptadecan-9-yl 8-((3-((2-(methylamino)-3,4-dioxocyclobut-1-en-1-yl)amino)propyl)(8-oxo-8-(undecan-3-yloxy)octyl)amino)octanoate

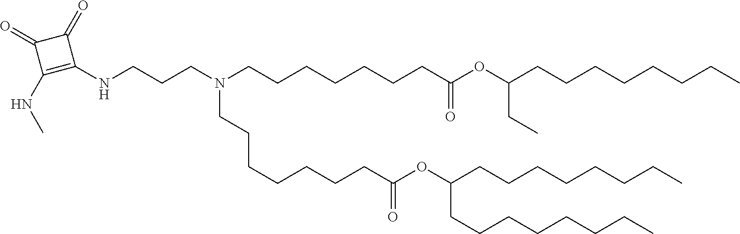 [SMILES: CCCCCCCCC(CC)OC(=O)CCCCCCCN(CCCCCCCC(=O)OC(CCCCCCCC)CCCCCCCC)CCCNc1c(NC)c(=O)c1=O] [0889] Chemical Formula: C₅₂H₉₇N₃O₆
Molecular Weight: 860.36

Compound I-301 was prepared analogously to compound 182 except that heptadecan-9-yl 8-((3-aminopropyl)(8-oxo-8-(undecan-3-yloxy)octyl)amino)octanoate (500 mg, 0.66 mmol) was used instead of heptadecan-9-yl 8-((3-aminopropyl)(8-(nonyloxy)-8-oxooctyl)amino)octanoate. Following an aqueous workup the residue was purified by silica gel chromatography (0-50% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-oxo-8-(undecan-3-yloxy)octyl)amino)octanoate (180 mg, 32%) as a white waxy solid. HPLC/UV (254 nm): RT=6.77 min. MS (CI): m/z (MH⁺) 860.7 for C₅₂H₉₇N₃O₆. ¹H NMR (300 MHz, CDCl₃): δ ppm 4.86-4.79 (m, 2H); 3.66 (bs, 2H); 3.25 (d, 3H, J=4.9 Hz); 2.56-2.52 (m, 2H); 2.42-2.37 (m, 4H); 2.28 (dd, 4H, J=2.7 Hz, 7.4 Hz); 1.78-1.68 (m, 3H); 1.64-1.50 (m, 16H); 1.48-1.38 (m, 6H); 1.32-1.18 (m, 43H); 0.88-0.84 (m. 12H).

(ii) Cholesterol/Structural Lipids

The immune cell delivery LNPs described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. Examples of structural lipids include, but are not limited to, the following:

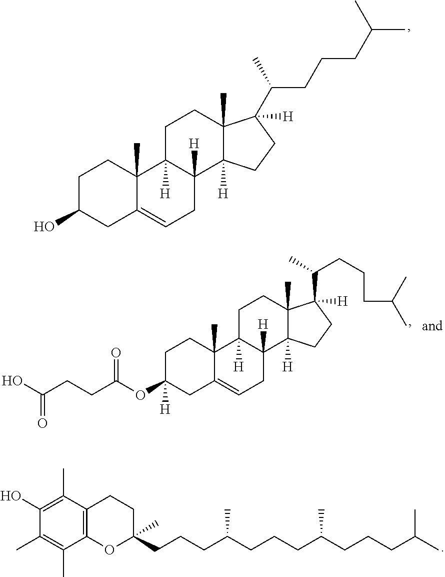

The immune cell delivery LNPs described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. Structural lipids can include, but are not limited to, sterols (e.g., phytosterols or zoosterols).

In certain embodiments, the structural lipid is a steroid. For example, sterols can include, but are not limited to, cholesterol, β-sitosterol, fecosterol, ergosterol, sitosterol, campesterol, stigmasterol, brassicasterol, ergosterol, tomatidine, tomatine, ursolic acid, alpha-tocopherol, or any one of compounds S1-148 in Tables 1-16 herein.

In certain embodiments, the structural lipid is cholesterol. In certain embodiments, the structural lipid is an analog of cholesterol.

In certain embodiments, the structural lipid is alpha-tocopherol.

In an aspect, the structural lipid of the invention features a compound having the structure of Formula SI:

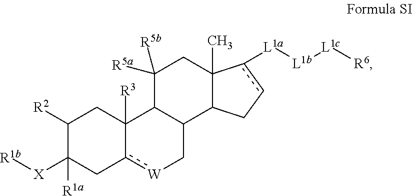

where

R^{1a} is H, optionally substituted C₁-C₆ alkyl, optionally substituted C₂-C₆ alkenyl, or optionally substituted C₂-C₆ alkynyl;

X is O or S;

R^{1b} is H, optionally substituted C₁-C₆ alkyl, or

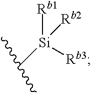

each of R^{b1}, R^{b2}, and R^{b3} is, independently, optionally substituted C₁-C₆ alkyl or optionally substituted C₆-C₁₀ aryl;

R² is H or OR^{A}, where R^{A} is H or optionally substituted C₁-C₆ alkyl;

R³ is H or

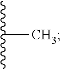

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

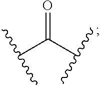 [SMILES: *C(*)=O]

L^{1a} is absent,

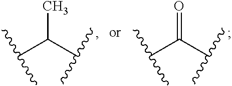

L^{1b} is absent,

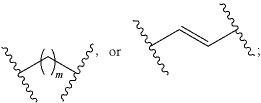

m is 1, 2, or 3;

L^{1c} is absent, and

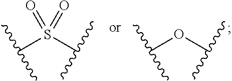

R⁶ is optionally substituted C₃-C₁₀ cycloalkyl, optionally substituted C₃-C₁₀ cycloalkenyl, optionally substituted C₆-C₁₀ aryl, optionally substituted C₂-C₉ heterocyclyl, or optionally substituted C₂-C₉ heteroaryl,

or a pharmaceutically acceptable salt thereof.

In some embodiments, the compound has the structure of Formula SIa:

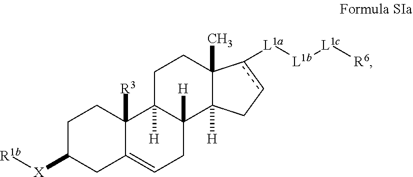

or a pharmaceutically acceptable salt thereof.

In some embodiments, the compound has the structure of Formula SIb:

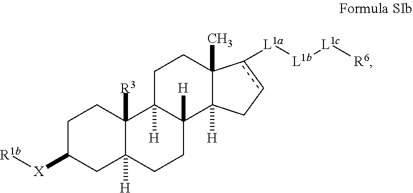

or a pharmaceutically acceptable salt thereof.

In some embodiments, the compound has the structure of Formula SIc:

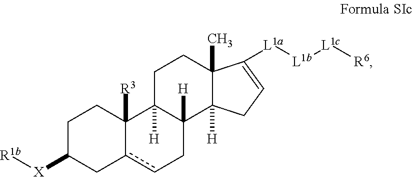

or a pharmaceutically acceptable salt thereof.

In some embodiments, the compound has the structure of Formula SId:

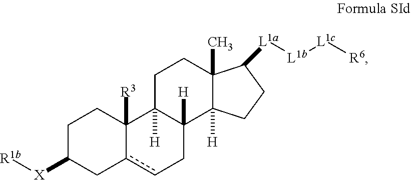

or a pharmaceutically acceptable salt thereof.

In some embodiments, L^{1a} is absent. In some embodiments, L^{1a} is

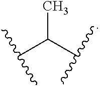 [SMILES: *C(*)C]

In some embodiments, L^{1a} is

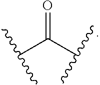 [SMILES: *C(*)=O]

In some embodiments, L^{1b} is absent. In some embodiments, L^{1b} is

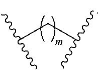

In some embodiments, L^{1b} is

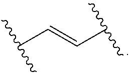 [SMILES: */C=C/*]

In some embodiments, m is 1 or 2. In some embodiments, m is 1. In some embodiments, m is 2.

In some embodiments, L^{1c} is absent. In some embodiments, L^{1c} is

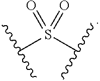 [SMILES: *S(*)(=O)=O]

In some embodiments, L^{1c} is

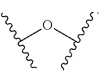 [SMILES: *O*]

In some embodiments, R⁶ is optionally substituted C₆-C₁₀ aryl.

In some embodiments, R⁶ is

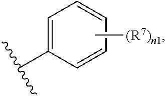

where

n1 is 0, 1, 2, 3, 4, or 5; and

each R⁷ is, independently, halo or optionally substituted C₁-C₆ alkyl.

In some embodiments, each R⁷ is, independently,

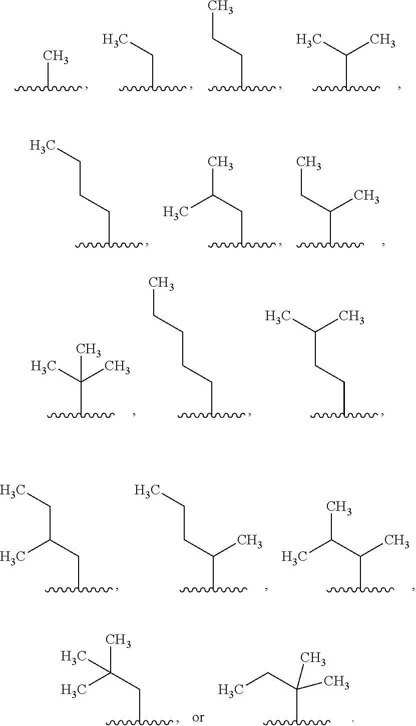

In some embodiments, n1 is 0, 1, or 2. In some embodiments, n is 0. In some embodiments, n1 is 1. In some embodiments, n1 is 2.

In some embodiments, R⁶ is optionally substituted C₃-C₁₀ cycloalkyl.

In some embodiments, R⁶ is optionally substituted C₃-C₁₀ monocycloalkyl.

In some embodiments, R⁶ is

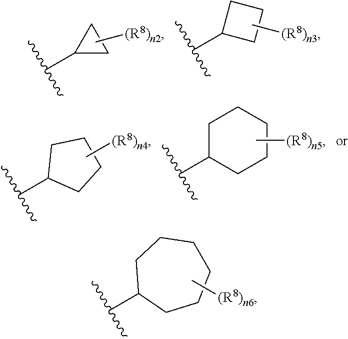

where
n2 is 0, 1, 2, 3, 4, or 5;
n3 is 0, 1, 2, 3, 4, 5, 6, or 7;
n4 is 0, 1, 2, 3, 4, 5, 6, 7, 8, or 9;
n5 is 0, 1, 2, 3, 4, 5, 6, 7, 8, 9, 10, or 11;
n6 is 0, 1, 2, 3, 4, 5, 6, 7, 8, 9, 10, 11, 12, or 13; and
each R⁸ is, independently, halo or optionally substituted C₁-C₆ alkyl.

In some embodiments, each R⁸ is, independently,

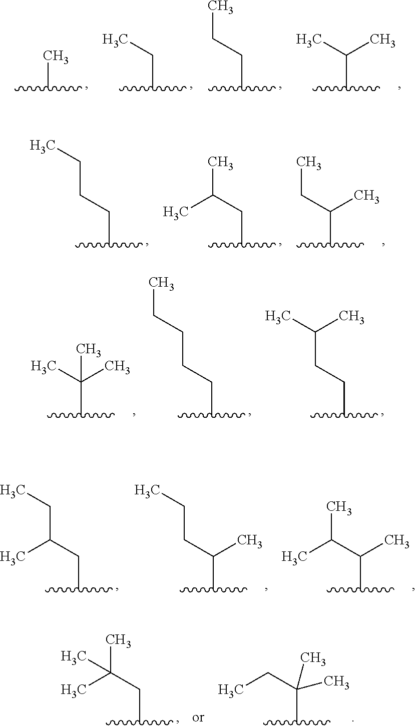

In some embodiments, R⁶ is optionally substituted C₃-C₁₀ polycycloalkyl.

In some embodiments, R⁶ is

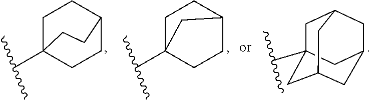

In some embodiments, R⁶ is optionally substituted C₃-C₁₀ cycloalkenyl.

In some embodiments, R⁶ is

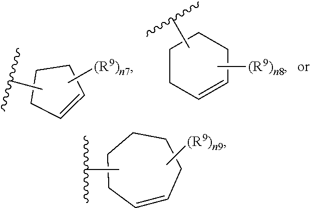

where
n7 is 0, 1, 2, 3, 4, 5, 6, or 7;
n8 is 0, 1, 2, 3, 4, 5, 6, 7, 8, or 9;
n9 is 0, 1, 2, 3, 4, 5, 6, 7, 8, 9, 10, or 11; and
each R⁹ is, independently, halo or optionally substituted C₁-C₆ alkyl.

In some embodiments, R⁶ is

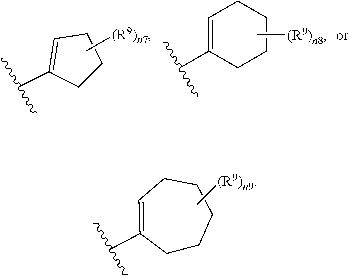

In some embodiments, each R⁹ is, independently,

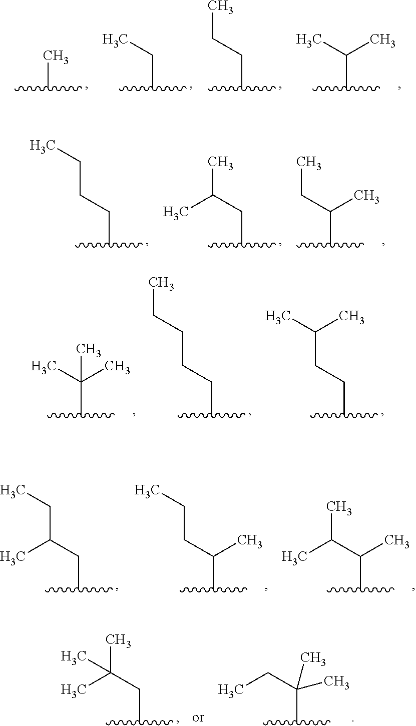

In some embodiments, R⁶ is optionally substituted C₂-C₉ heterocyclyl.

In some embodiments, R⁶ is

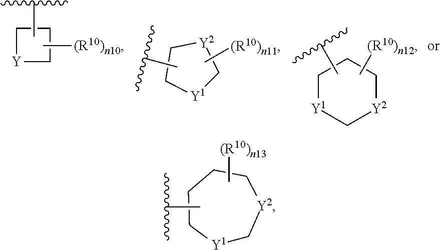

where

n10 is 0, 1, 2, 3, 4, or 5;

n11 is 0, 1, 2, 3, 4, or 5;

n12 is 0, 1, 2, 3, 4, 5, 6, or 7;

n13 is 0, 1, 2, 3, 4, 5, 6, 7, 8, or 9;

each R¹⁰ is, independently, halo or optionally substituted C₁-C₆ alkyl; and

each of Y¹ and Y² is, independently, O, S, NR^{B}, or CR^{11a}R^{11b},

where R^{B} is H or optionally substituted C₁-C₆ alkyl;

each of R^{11a} and R^{11b} is, independently, H, halo, or optionally substituted C₁-C₆ alkyl; and

if Y² is CR^{11a}R^{11b}, then Y¹ is O, S, or NR^{B}.

In some embodiments, Y¹ is O.

In some embodiments, Y² is O. In some embodiments, Y² is CR^{1la}R^{11b}.

In some embodiments, each R¹⁰ is, independently,

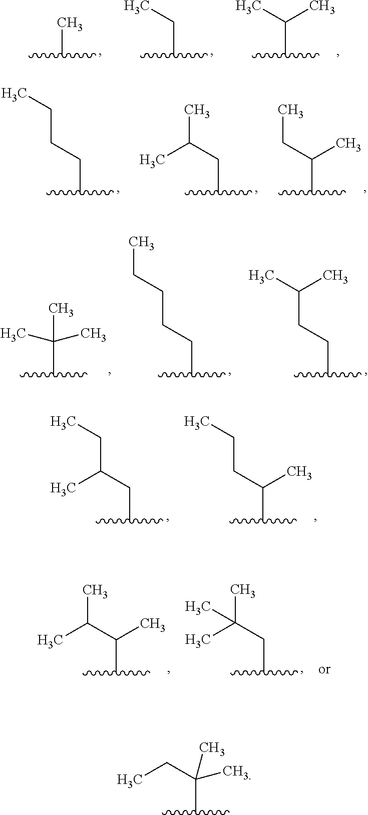

In some embodiments, R⁶ is optionally substituted C₂-C₉ heteroaryl.

In some embodiments, R⁶ is

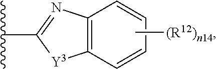

where

Y³ is NR^{C}, O, or S

n14 is 0, 1, 2, 3, or 4;

R^{C} is H or optionally substituted C₁-C₆ alkyl; and

each R¹² is, independently, halo or optionally substituted C₁-C₆ alkyl.

In some embodiments, R⁶ is

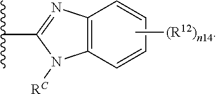

In some embodiments, R⁶ is

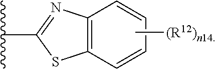

In an aspect, the structural lipid of the invention features a compound having the structure of Formula SII:

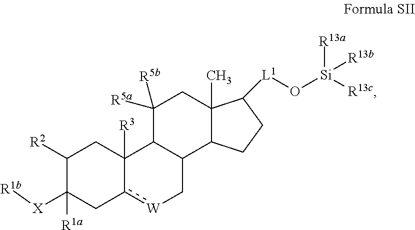

where
R^{1a} is H, optionally substituted C₁-C₆ alkyl, optionally substituted C₂-C₆ alkenyl, or optionally substituted C₂-C₆ alkynyl;

X is O or S;
R^{1b} is H or optionally substituted C₁-C₆ alkyl;
R² is H or OR^{A}, where R^{A} is H or optionally substituted C₁-C₆ alkyl;
R³ is H or

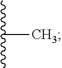

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{4a} and R^{5b} is, independently, H or OR^{A}, or R^{5a} and R^{5b}, together with the atom to

which each is attached, combine to form

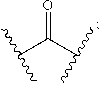 [SMILES: *C(*)=O]

L¹ is optionally substituted C₁-C₆ alkylene; and

each of R^{13a}, R^{13b}, and R^{13c} is, independently, optionally substituted C₁-C₆ alkyl or optionally substituted C₆-C₁₀ aryl,

or a pharmaceutically acceptable salt thereof.

In some embodiments, the compound has the structure of Formula SIIa:

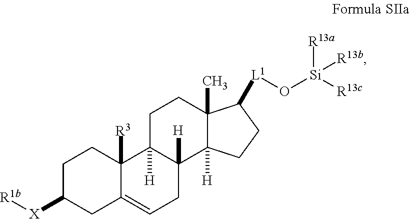

or a pharmaceutically acceptable salt thereof.

In some embodiments, the compound has the structure of Formula Silly

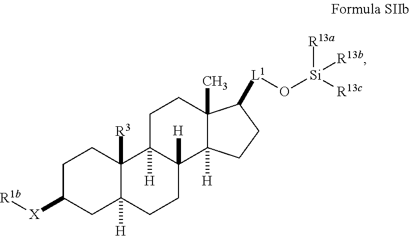

or a pharmaceutically acceptable salt thereof.

In some embodiments, L¹ is

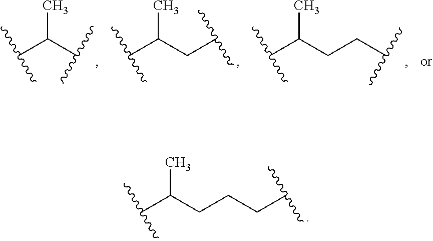

In some embodiments, each of R^{13a}, R^{13b}, and R^{13c} is, independently,

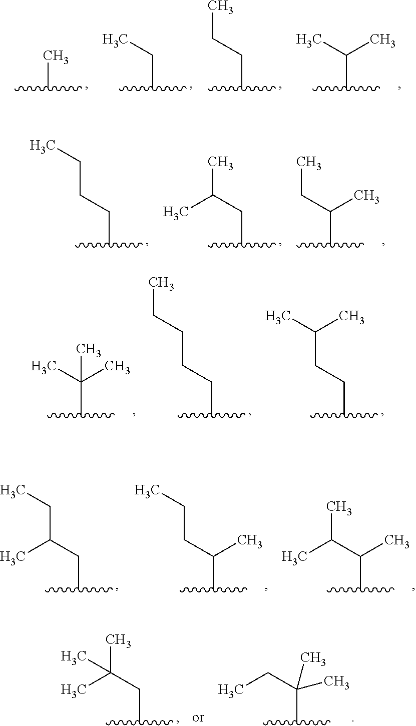

In an aspect, the structural lipid of the invention features a compound having the structure of Formula SIII:

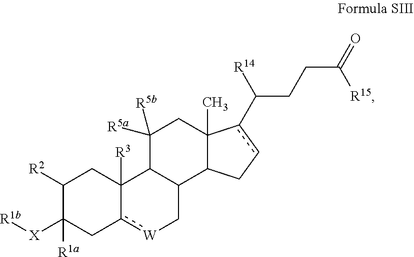

where
R^{1a} is H, optionally substituted C₁-C₆ alkyl, optionally substituted C₂-C₆ alkenyl, or optionally substituted C₂-C₆ alkynyl;

X is O or S;
R^{1b} is H or optionally substituted C₁-C₆ alkyl;
R² is H or OR^{A}, where R^{A} is H or optionally substituted C₁-C₆ alkyl;

R³ is H or

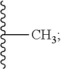

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, hydroxyl, optionally substituted C₁-C₆ alkyl, —OS(O)₂R^{4c}, where R^{4c} is optionally substituted C₁-C₆ alkyl or optionally substituted C₆-C₁₀ aryl;

each of R^{5a} and R^{5b} is, independently, H or OR^{A}, or R^{5a} and R^{5b}, together with the atom to which each is attached, combine to form

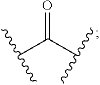 [SMILES: *C(*)=O]

R¹⁴ is H or C₁-C₆ alkyl; and

R¹⁵ is

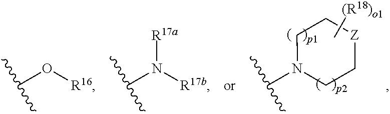

where

R¹⁶ is H or optionally substituted C₁-C₆ alkyl;

R^{17b} is H, OR^{17c}, optionally substituted C₆-C₁₀ aryl, or optionally substituted C₁-C₆ alkyl;

R^{17c} is H or optionally substituted C₁-C₆ alkyl;

o1 is 0, 1, 2, 3, 4, 5, 6, 7, or 8;

p1 is 0, 1, or 2;

p2 is 0, 1, or 2;

Z is CH₂ O, S, or NR^{D}, where R^{D} is H or optionally substituted C₁-C₆ alkyl; and

each R¹⁸ is, independently, halo or optionally substituted C₁-C₆ alkyl,

or a pharmaceutically acceptable salt thereof.

In some embodiments, the compound has the structure of Formula SIIIa:

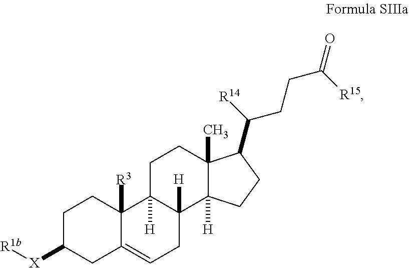

or a pharmaceutically acceptable salt thereof.

In some embodiments, the compound has the structure of Formula SIIIb:

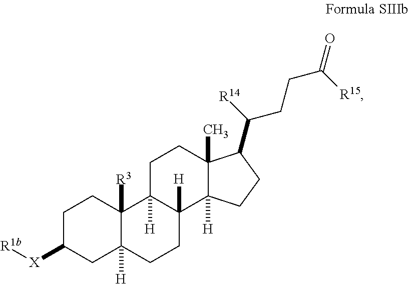

or a pharmaceutically acceptable salt thereof.

In some embodiments, R¹⁴ is H,

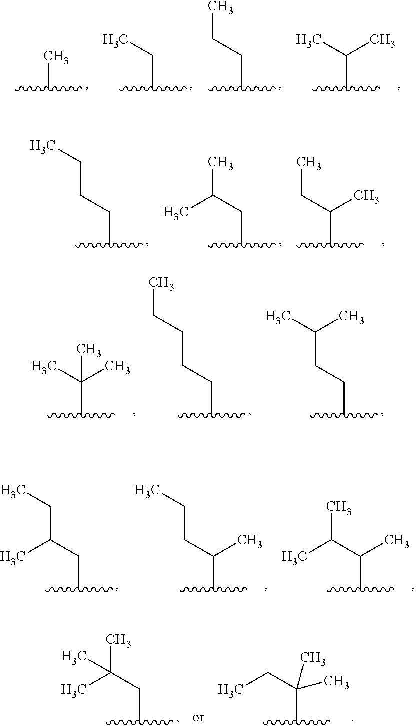

In some embodiments, R¹⁴ is

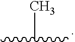

In some embodiments, R¹⁵ is

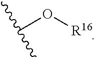

In some embodiments, R¹⁵ is

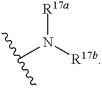

In some embodiments, R¹⁶ is H. In some embodiments, R¹⁶ is

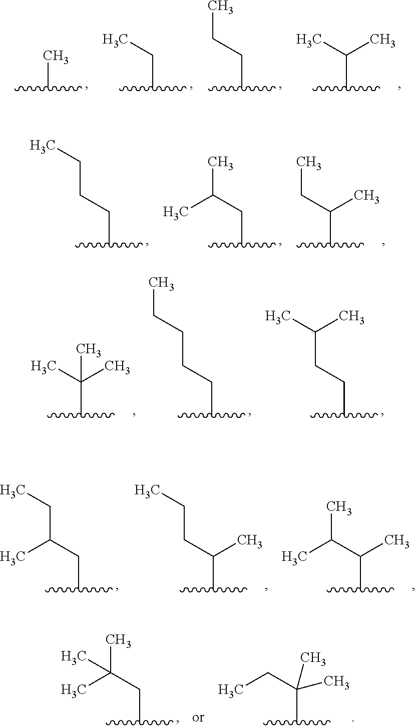

In some embodiments, R^{17a} is H. In some embodiments, R^{17a} is optionally substituted C₁-C₆ alkyl.

In some embodiments, R^{17b} is H. In some embodiments, R^{17b} optionally substituted C₁-C₆ alkyl. In some embodiments, R^{17b} is OR^{17c}.

In some embodiments, R^{17c} is H,

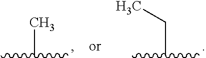

In some embodiments, R^{17c} is H. In some embodiments, R^{17c} is

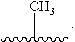

In some embodiments, R¹⁵ is

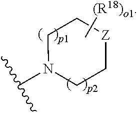

In some embodiments, each R¹⁸ is, independently,

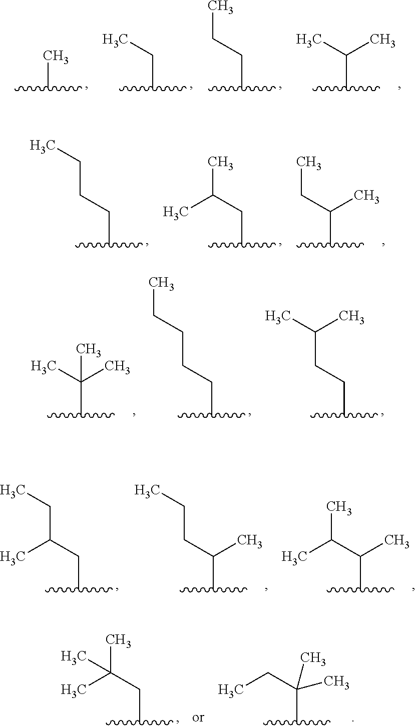

In some embodiments, Z is CH₂. In some embodiments, Z is O. In some embodiments, Z is NR^{D}.

In some embodiments, o1 is 0, 1, 2, 3, 4, 5, or 6.

In some embodiments, o1 is 0. In some embodiments, o1 is 1. In some embodiments, o1 is 2. In some embodiments, o1 is 3. In some embodiments, o1 is 4. In some embodiments, o1 is 5. In some embodiments, o1 is 6.

In some embodiments, p1 is 0 or 1. In some embodiments, p1 is 0. In some embodiments, p1 is 1.

In some embodiments, p2 is 0 or 1. In some embodiments, p2 is 0. In some embodiments, p2 is 1.

In an aspect, the structural lipid of the invention features a compound having the structure of Formula SIV:

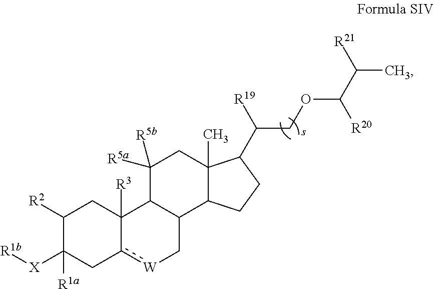

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

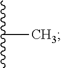

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

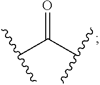 [SMILES: *C(*)=O]

s is 0 or 1;

R¹⁹ is H or C₁-C₆ alkyl;

R²⁰ is C₁-C₆ alkyl;

R²¹ is H or C₁-C₆ alkyl,

or a pharmaceutically acceptable salt thereof.

In some embodiments, the compound has the structure of Formula SIVa:

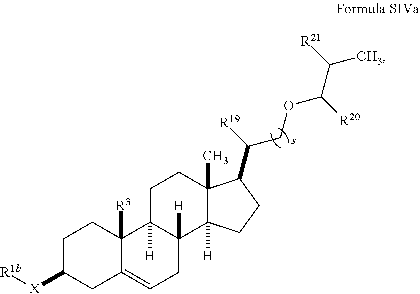

or a pharmaceutically acceptable salt thereof.

In some embodiments, the compound has the structure of Formula SIVb:

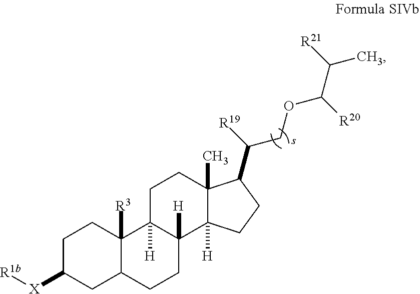

or a pharmaceutically acceptable salt thereof.

In some embodiments, R¹⁹ is H,

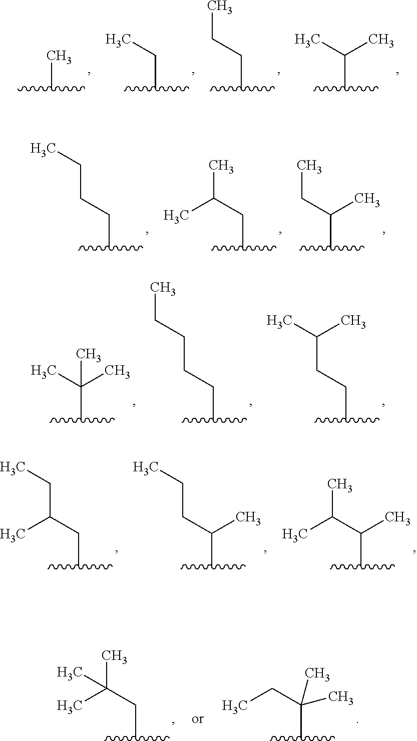

In some embodiments, R¹⁹ is

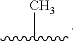

In some embodiments, R²⁰ is,

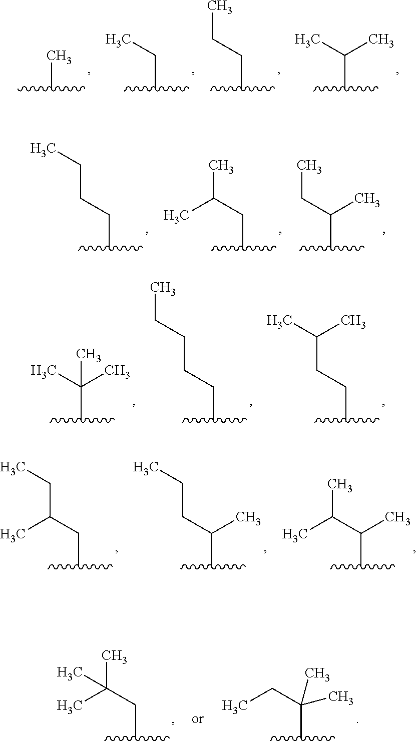

In some embodiments, R²¹ is H,

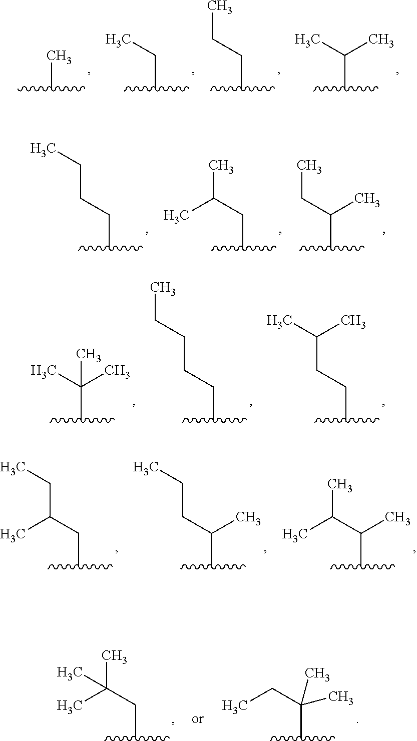

In an aspect, the structural lipid of the invention features, a compound having the structure of Formula SV:

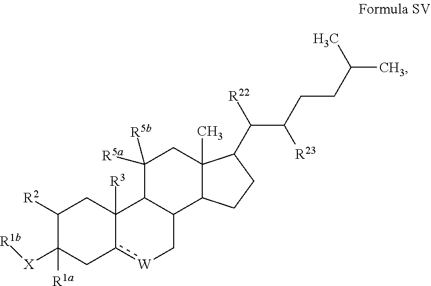

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

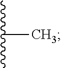

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{4b} and R^{5b}, together with the atom to which each is attached, combine to form

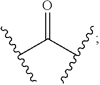 [SMILES: *C(*)=O]

R²² is H or C₁-C₆ alkyl; and

R²³ is halo, hydroxyl, optionally substituted C₁-C₆ alkyl, or optionally substituted C₁-C₆ heteroalkyl,

or a pharmaceutically acceptable salt thereof.

In some embodiments, the compound has the structure of Formula SVa:

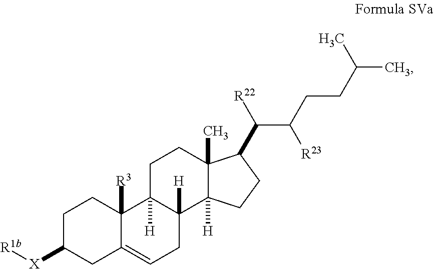

or a pharmaceutically acceptable salt thereof.

In some embodiments, the compound has the structure of Formula SVb:

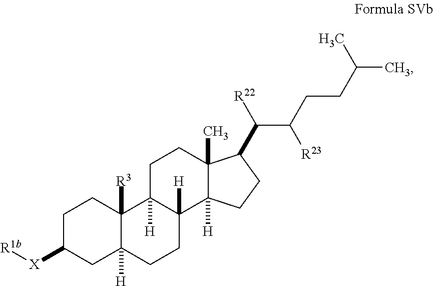

or a pharmaceutically acceptable salt thereof.

In some embodiments, R²² is H,

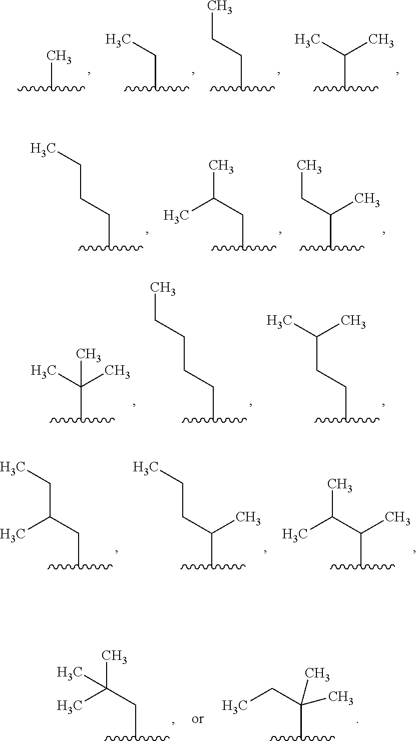

In some embodiments, R²² is

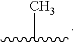

In some embodiments, R²³ is

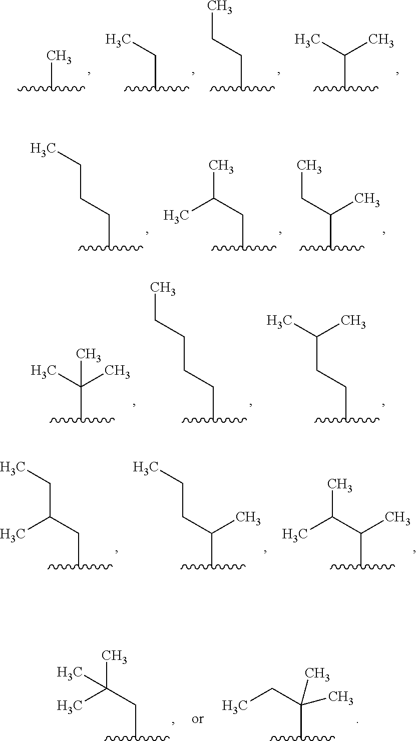

In an aspect, the structural lipid of the invention features a compound having the structure of Formula SVI:

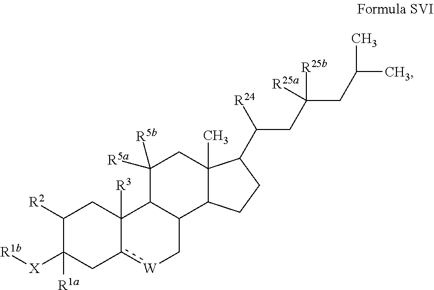

where
R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

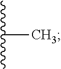

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

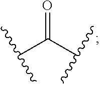 [SMILES: *C(*)=O]

R²⁴ is H or C₁-C₆ alkyl; and

each of R^{25a} and R^{25b} is C₁-C₆ alkyl,

or a pharmaceutically acceptable salt thereof.

In some embodiments, the compound has the structure of Formula SVIa:

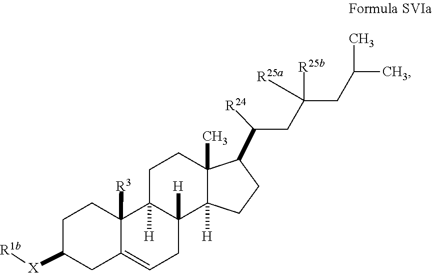

or a pharmaceutically acceptable salt thereof.

In some embodiments, the compound has the structure of Formula SVIb:

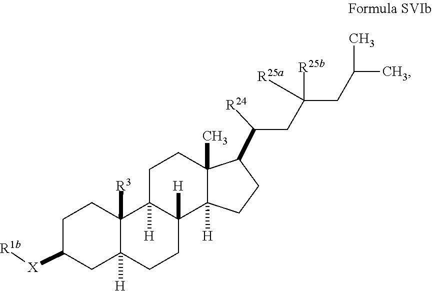

or a pharmaceutically acceptable salt thereof.

In some embodiments, R²⁴ is H,

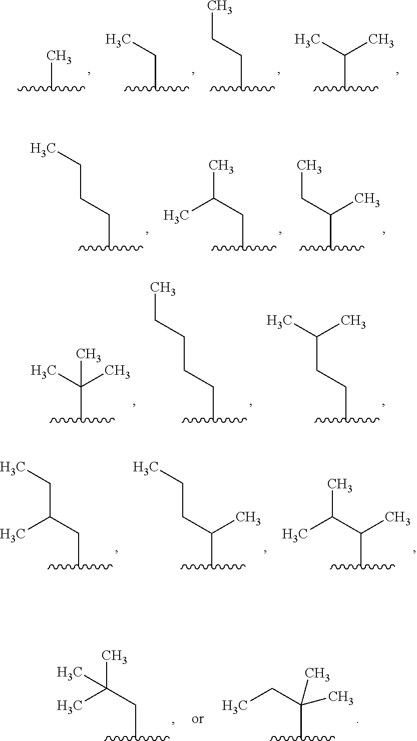

In some embodiments, R²⁴ is

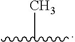

In some embodiments, each of R^{25a} and R^{25b} is, independently,

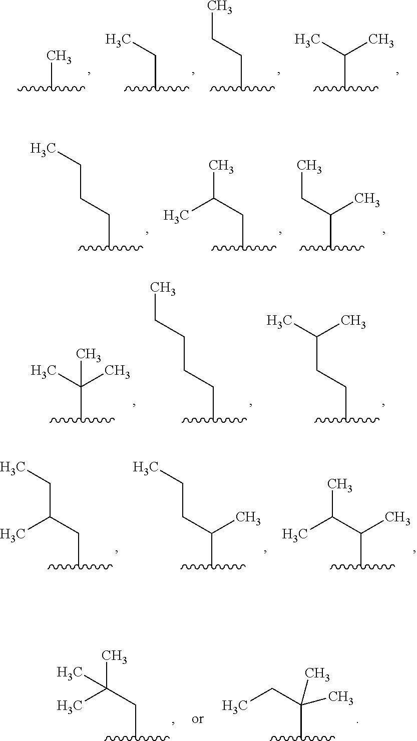

In an aspect, the structural lipid of the invention features a compound having the structure of Formula SVII:

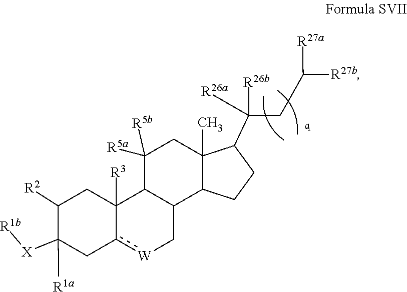

where

R^{1a} is H, optionally substituted C₂-C₆ alkyl, optionally substituted C₂-C₆ alkenyl, optionally substituted C₂-C₆ alkynyl, or

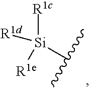

where each of R^{1c}, R^{1d}, and R^{1e} is, independently, optionally substituted C₁-C₆ alkyl or optionally substituted C₆-C₁₀ aryl;

X is O or S;

R^{1b} is H or optionally substituted C₁-C₆ alkyl;

R² is H or OR^{A}, where R^{A} is H or optionally substituted C₁-C₆ alkyl;

R³ is H or

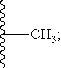

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

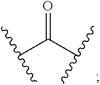 [SMILES: *C(*)=O]

q is 0 or 1;

each of R^{26a} and R^{26b} is, independently, H or optionally substituted C₁-C₆ alkyl, or R^{26a} and R^{26b}, together with the atom to which each is attached, combine to form

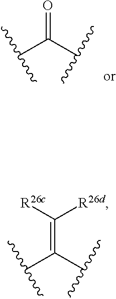

where each of R^{26c} and R²⁶ is, independently, H or optionally substituted C₁-C₆ alkyl; and

each of R^{27a} and R^{27b} is H, hydroxyl, or optionally substituted C₁-C₆ alkyl,

or a pharmaceutically acceptable salt thereof.

In some embodiments, the compound has the structure of Formula SVIIa:

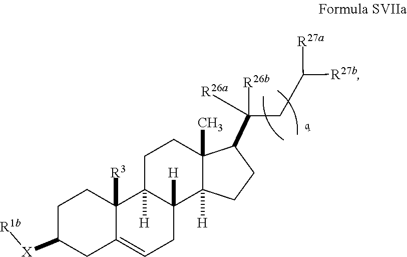

or a pharmaceutically acceptable salt thereof.

In some embodiments, the compound has the structure of Formula SVIIb:

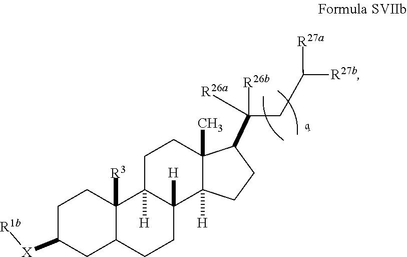

or a pharmaceutically acceptable salt thereof.

In some embodiments, R^{26a} and R^{26b} is, independently, H,

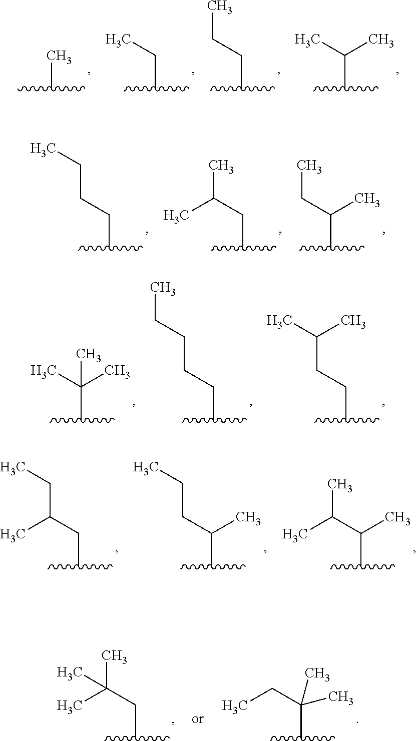

In some embodiments, R^{26a} and R^{26b}, together with the atom to which each is attached, combine to form

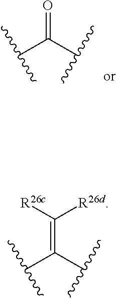

In some embodiments, R^{26a} and R^{26b}, together with the atom to which each is attached, combine to form

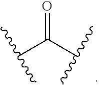 [SMILES: *C(*)=O]

In some embodiments. R^{26a} and R^{26b}, together with the atom to which each is attached, combine to form

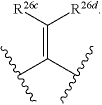

In some embodiments, where each of R^{26c} and R²⁶ is, independently, H,

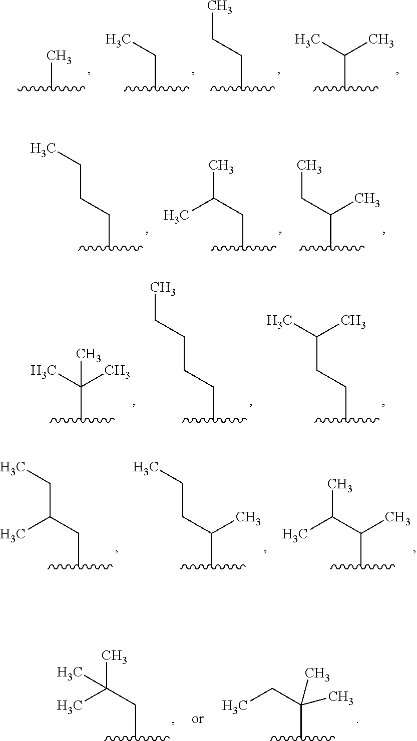

In some embodiments, each of R^{27a} and R^{27b} is H, hydroxyl, or optionally substituted C₁-C₃ alkyl.

In some embodiments, each of R^{27a} and R^{27b} is, independently, H, hydroxyl,

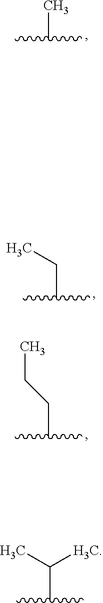

In an aspect, the structural lipid of the invention features a compound having the structure of Formula SVIII:

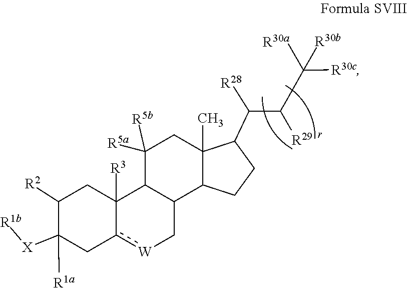

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A} where R^{A} is H or optionally substituted C₁-C₆ alkyl;

R³ is H or

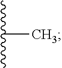

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

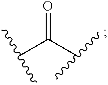 [SMILES: *C(*)=O]

R²⁸ is H or optionally substituted C₁-C₆ alkyl;

r is 1, 2, or 3;

each R²⁹ is, independently, H or optionally substituted C₁-C₆ alkyl; and

each of R^{30a}, R^{30b}, and R^{30c} is C₁-C₆ alkyl,

or a pharmaceutically acceptable salt thereof.

In some embodiments, the compound has the structure of Formula SVIIIa:

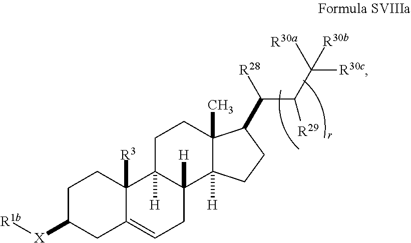

or a pharmaceutically acceptable salt thereof.

In some embodiments, the compound has the structure of Formula SVIIIb:

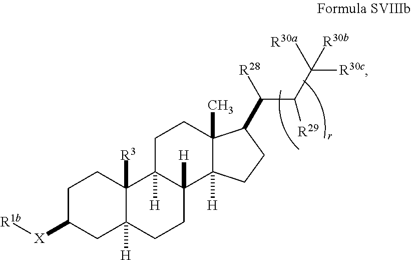

or a pharmaceutically acceptable salt thereof.

In some embodiments, R²⁸ is H,

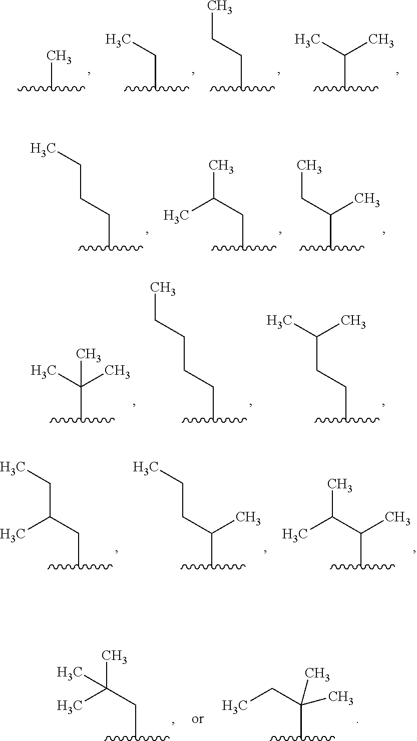

In some embodiments, R²⁸ is

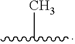

In some embodiments, each of R^{30a}, R^{30b}, and R^{30c} is, independently,

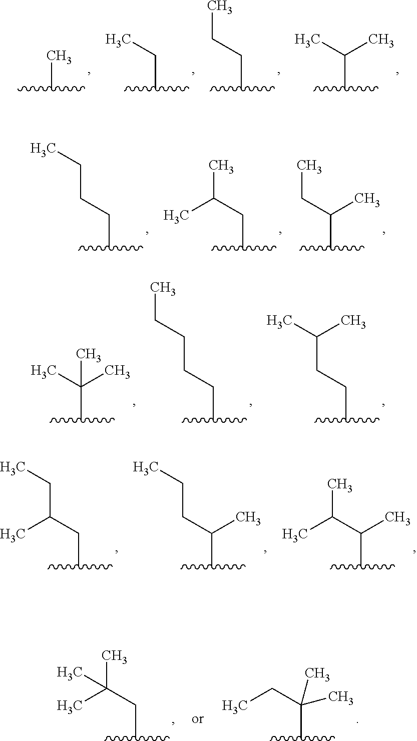

In some embodiments, r is 1. In some embodiments, r is 2. In some embodiments, r is 3.

In some embodiments, each R²⁹ is, independently, H,

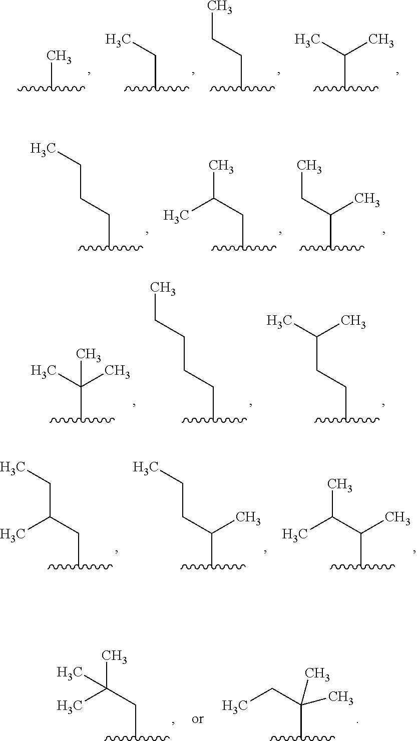

In some embodiments, each R²⁹ is, independently, H or

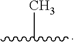

In an aspect, the structural lipid of the invention features a compound having the structure of Formula SIX:

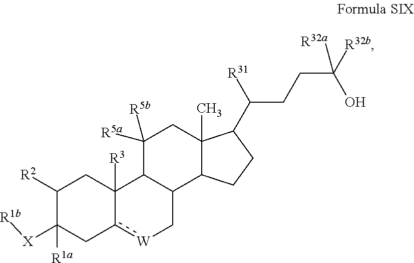

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

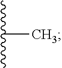

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

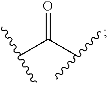 [SMILES: *C(*)=O]

R³¹ is H or C₁-C₆ alkyl; and

each of R^{32a} and R^{32b} is C₁-C₆ alkyl,

or a pharmaceutically acceptable salt thereof.

In some embodiments, the compound has the structure of Formula SIXa:

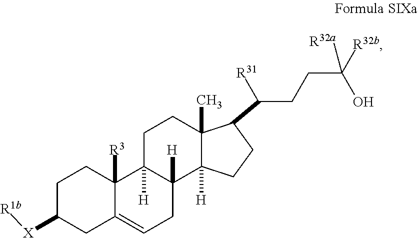

or a pharmaceutically acceptable salt thereof.

In some embodiments, the compound has the structure of Formula SIXb:

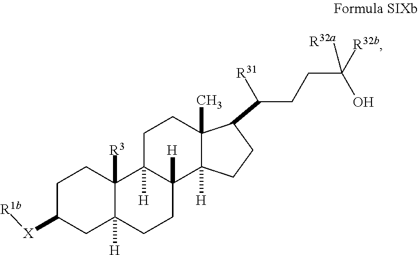

or a pharmaceutically acceptable salt thereof.

In some embodiments, R³¹ is H,

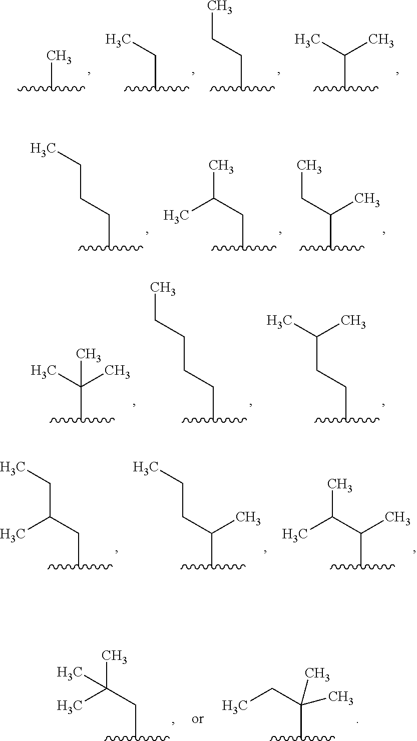

In some embodiments, R³¹ is

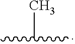

In some embodiments, each of R^{32a} and R^{32b} is, independently,

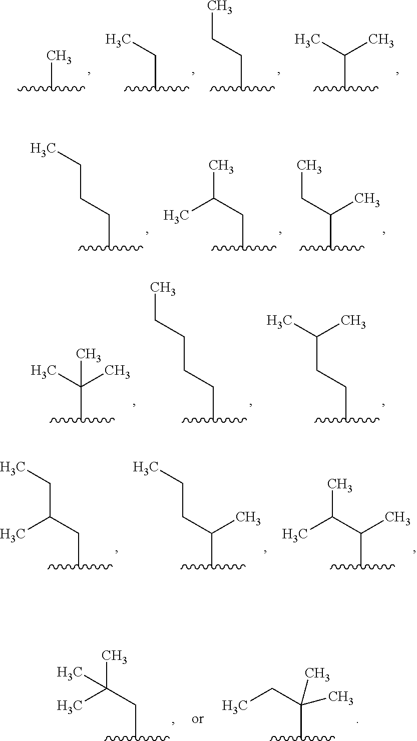

In an aspect, the structural lipid of the invention features a compound having the structure of Formula SX:

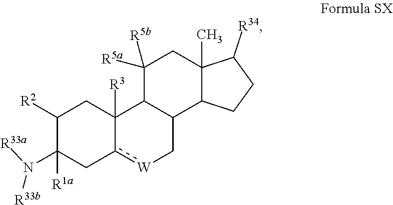

where
R^{1a} is H, optionally substituted C₁-C₆ alkyl, optionally substituted C₂-C₆ alkenyl, or optionally substituted C₂-C₆ alkynyl;

X is O or S;
R² is H or OR^{A}, where R^{A} is H or optionally substituted C₁-C₆ alkyl;
R³ is H or

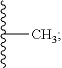

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form

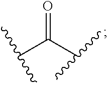 [SMILES: *C(*)=O]

R^{33a} is optionally substituted C₁-C₆ alkyl or

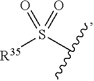

where R³⁵ is optionally substituted C₁-C₆ alkyl or optionally substituted C₆-C₁₀ aryl;

R^{33b} is H or optionally substituted C₁-C₆ alkyl; or

R³⁵ and R^{33b}, together with the atom to which each is attached, form an optionally substituted C₃-C₉ heterocyclyl; and

R³⁴ is optionally substituted C₁-C₆ alkyl or optionally substituted C₁-C₆ heteroalkyl,

or a pharmaceutically acceptable salt thereof.

In some embodiments, the compound has the structure of Formula SXa:

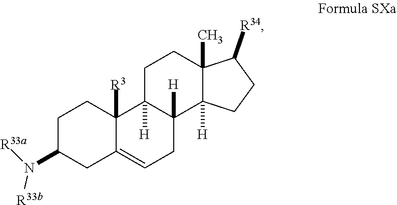

or a pharmaceutically acceptable salt thereof.

In some embodiments, the compound has the structure of Formula SXb:

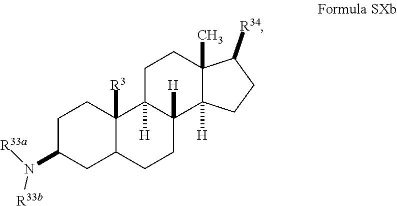

or a pharmaceutically acceptable salt thereof.

In some embodiments, R^{33a} is

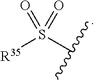

In some embodiments, R³⁵ is

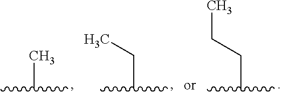

In some embodiments, R³⁵ is

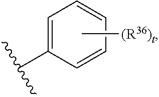

where
t is 0, 1, 2, 3, 4, or 5; and
each R³⁶ is, independently, halo, hydroxyl, optionally substituted C₁-C₆ alkyl, or optionally substituted C₁-C₆ heteroalkyl.

In some embodiments, R³⁴ is

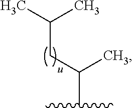

where u is 0, 1, 2, 3, or 4.

In some embodiments, u is 3 or 4.

In an aspect, the structural lipid of the invention features a compound having the structure of Formula SXI:

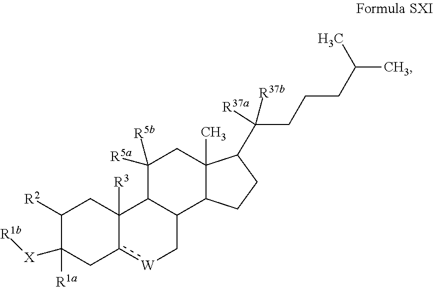

where
R^{1a} is H, optionally substituted C₁-C₆ alkyl, optionally substituted C₂-C₆ alkenyl, or optionally substituted C₂-C₆ alkynyl;

X is O or S;
R² is H or OR^{A}, where R^{A} is H or optionally substituted C₁-C₆ alkyl;
R³ is H or

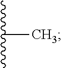

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form

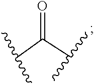 [SMILES: *C(*)=O]

and
each of R^{37a} and R^{37b} is, independently, optionally substituted C₁-C₆ alkyl, optionally substituted C₁-C₆ heteroalkyl, halo, or hydroxyl,

or a pharmaceutically acceptable salt thereof.

In some embodiments, the compound has the structure of Formula SXIa:

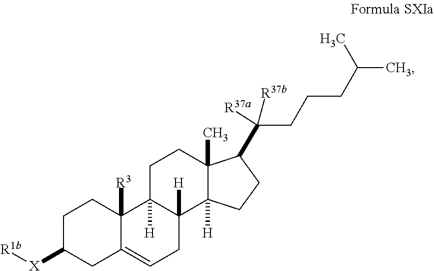

or a pharmaceutically acceptable salt thereof.

In some embodiments, the compound has the structure of Formula SXIb:

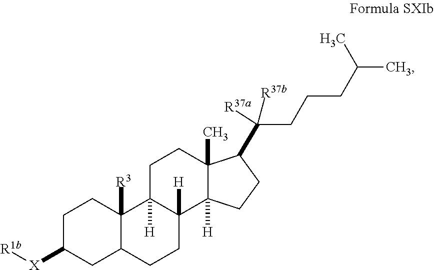

or a pharmaceutically acceptable salt thereof.

In some embodiments, R^{37a} is hydroxyl.

In some embodiments, R^{37b} is

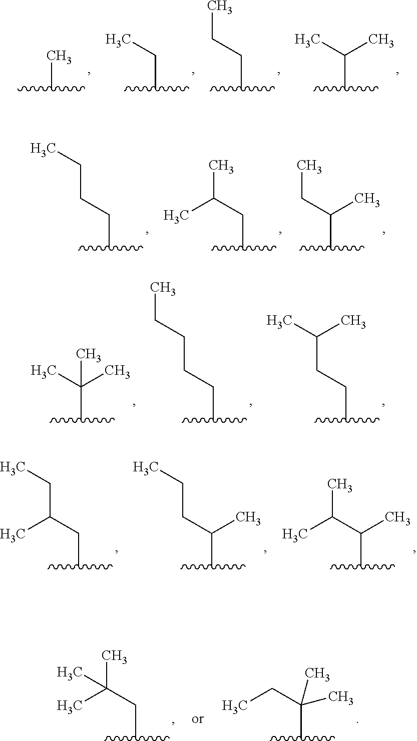

In an aspect, the structural lipid of the invention features a compound having the structure of Formula SXII:

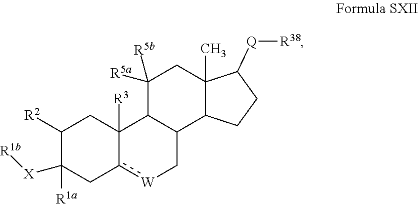

where
R^{1a} is H, optionally substituted C₁-C₆ alkyl, optionally substituted C₂-C₆ alkenyl, or optionally substituted C₂-C₆ alkynyl;

X is O or S;
R² is H or OR^{A}, where R^{A} is H or optionally substituted C₁-C₆ alkyl;

R³ is H or

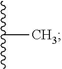

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form

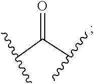 [SMILES: *C(*)=O]

and
Q is O, S, or NR^{E}, where R^{E} is H or optionally substituted C₁-C₆ alkyl; and
R³⁸ is optionally substituted C₁-C₆ alkyl,

or a pharmaceutically acceptable salt thereof.

In some embodiments, the compound has the structure of Formula SXIIa:

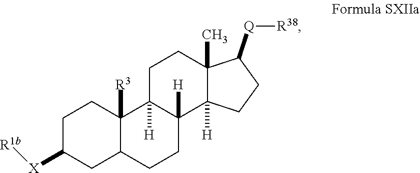

or a pharmaceutically acceptable salt thereof.

In some embodiments, the compound has the structure of Formula SXIIb:

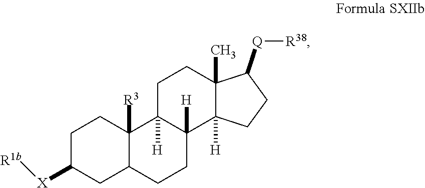

or a pharmaceutically acceptable salt thereof.

In some embodiments, Q is NR^{E}.

In some embodiments, R^{E} is H or

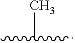

In some embodiments, R^{E} is H. In some embodiments, R^{E} is

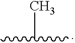

In some embodiments, R³⁸ is

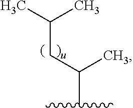

where u is 0, 1, 2, 3, or 4.

In some embodiments, X is O.

In some embodiments, R^{1a} is H or optionally substituted C₁-C₆ alkyl.

In some embodiments, R^{1a} is H.

In some embodiments, R^{1b} is H or optionally substituted C₁-C₆ alkyl.

In some embodiments, R^{1b} is H.

In some embodiments, R² is H.

In some embodiments, R^{4a} is H.

In some embodiments, R^{4b} is H.

In some embodiments, [symbol] represents a double bond.

In some embodiments, R³ is H. In some embodiments, R³ is

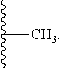

In some embodiments, R^{5a} is H.

In some embodiments, R^{5b} is H.

In an aspect, the invention features a compound having the structure of any one of compounds S-1-42, S-150, S-154, S-162-165, S-169-172 and S-184 in Table 10, or any pharmaceutically acceptable salt thereof. As used herein, “CMPD” refers to “compound.”

TABLE 10
Compounds of Formula SI

| CMPD | |
|---|---|
| No. S- | Structure |
| 1 | 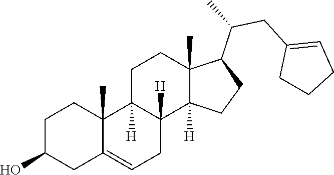 |
| 2 | 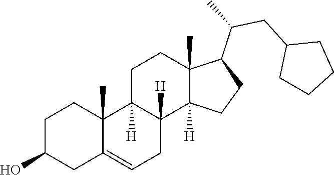 |
| 3 | 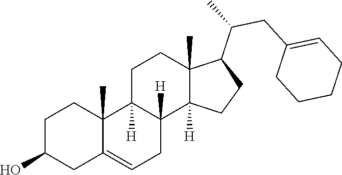 |
| 4 | 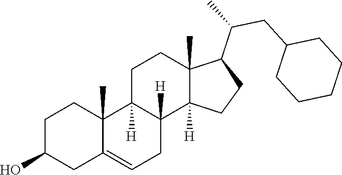 |
| 5 | 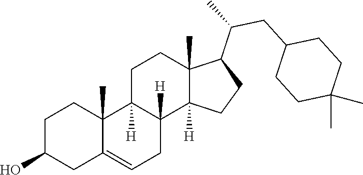 |
| 6 | 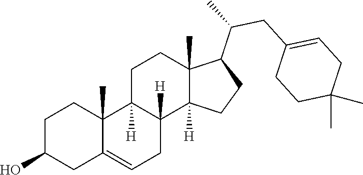 |
| 7 | 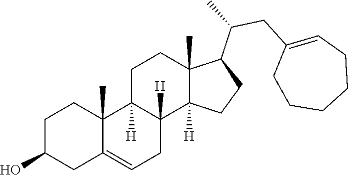 |
| 8 | 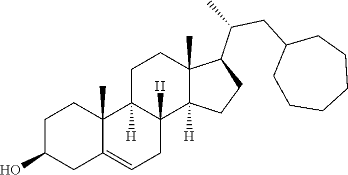 |
| 9 | 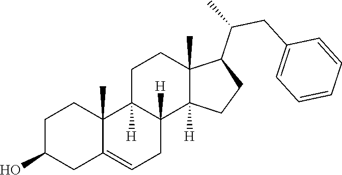 |
| 10 | 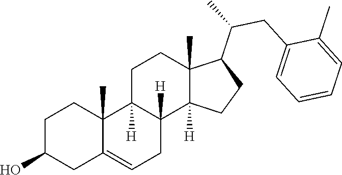 |
| 11 | 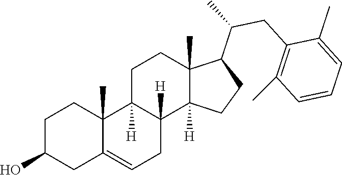 |
| 12 | 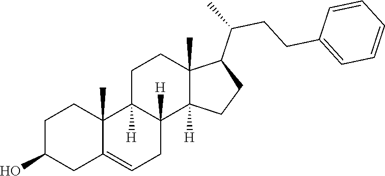 |
| 13 | 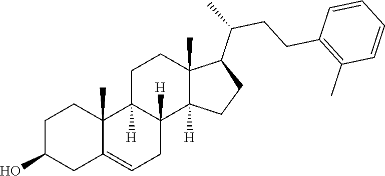 |
| 14 | 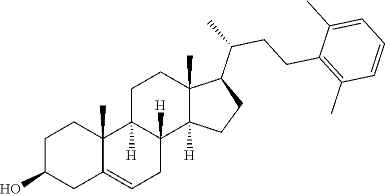 |
| 15 | 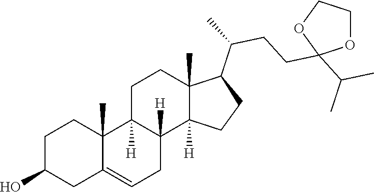 |
| 16 | 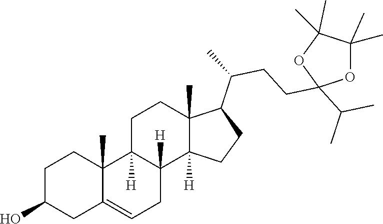 |
| 17 | 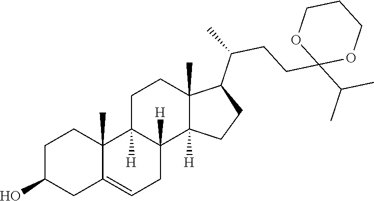 |
| 18 | 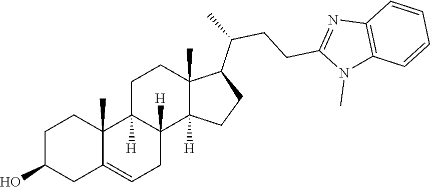 |
| 19 | 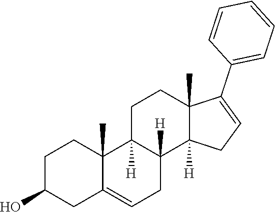 |
| 20 | 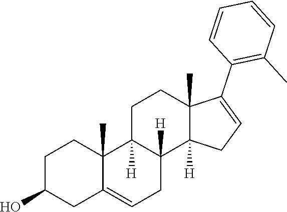 |
| 21 | 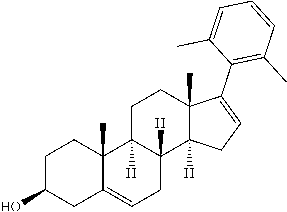 |
| 22 | 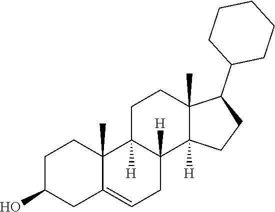 |
| 23 | 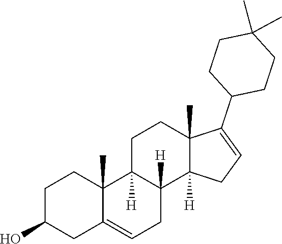 |
| 24 | 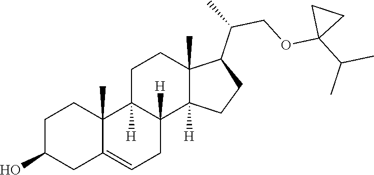 |
| 25 | 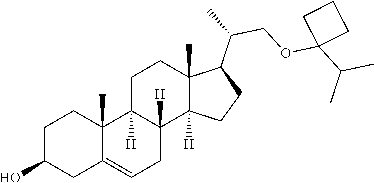 |
| 26 | 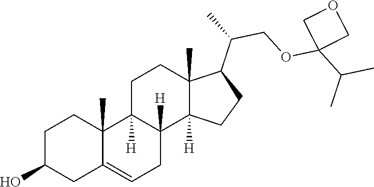 |
| 27 | 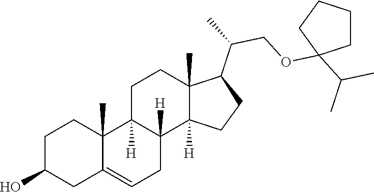 |
| 28 | 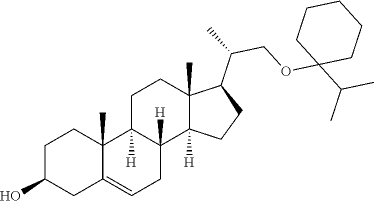 |
| 29 | 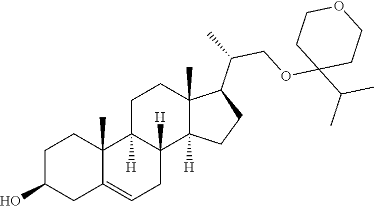 |
| 30 | 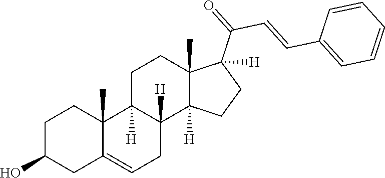 |
| 31 | 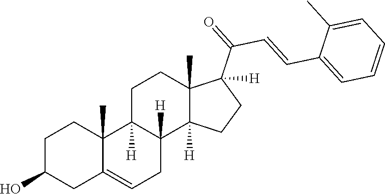 |
| 32 | 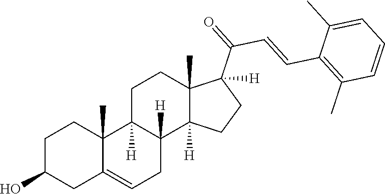 |
| 33 | 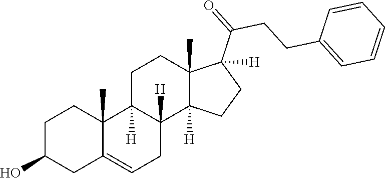 |
| 34 | 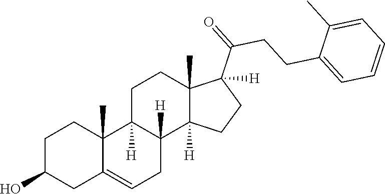 |
| 35 | 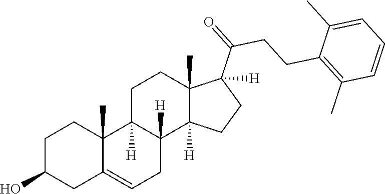 |
| 36 | 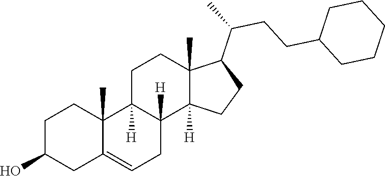 |
| 37 | 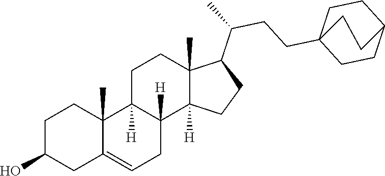 |
| 38 | 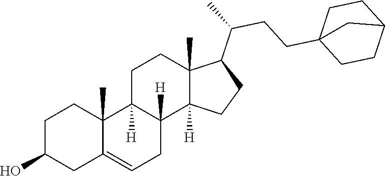 |
| 39 | 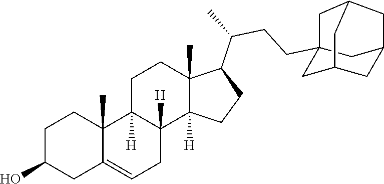 |
| 40 | 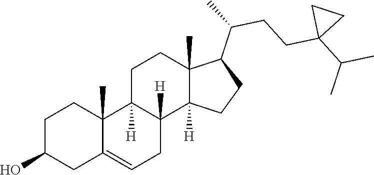 |
| 41 | 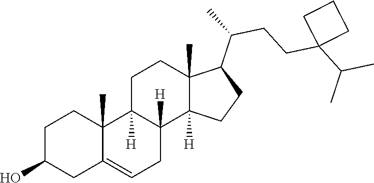 |
| 42 | 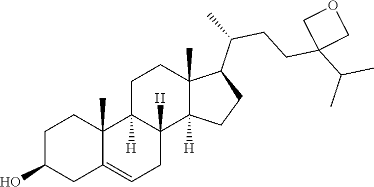 |
| 150 | 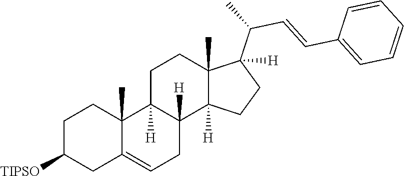 |
| 154 | 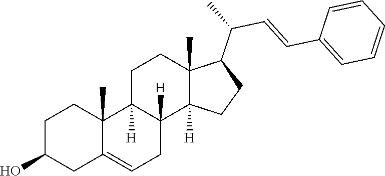 |
| 162 | 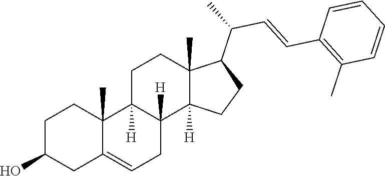 |
| 163 | 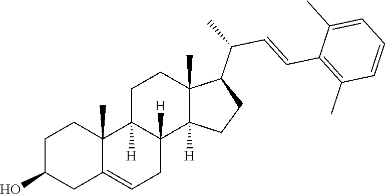 |
| 164 | 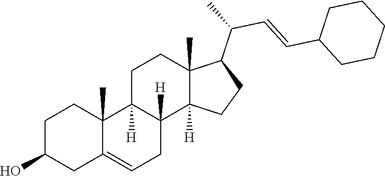 |
| 165 | 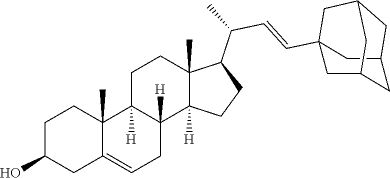 |
| 169 | 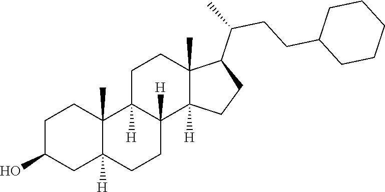 |
| 170 | 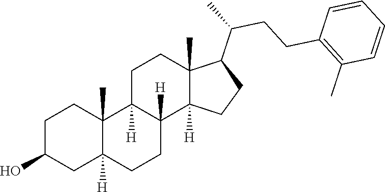 |
| 171 | 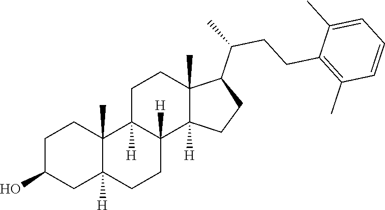 |
| 172 | 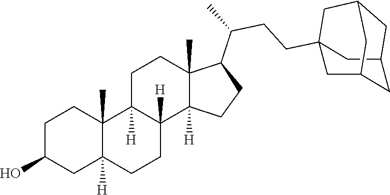 |
| 184 | 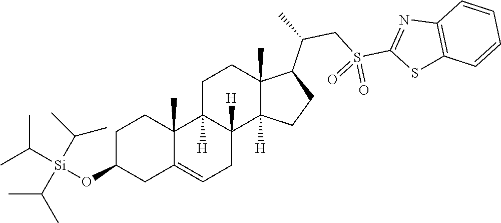 |

In an aspect, the invention features a compound having the structure of any one of compounds S-43-50 and S-175-178 in Table 11, or any pharmaceutically acceptable salt thereof.

TABLE 11
Compounds of Formula SII

| CMPD | |
|---|---|
| No. S- | Structure |
| 43 | 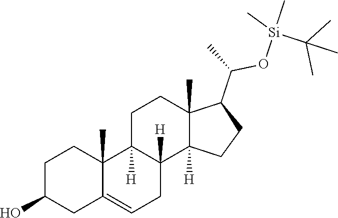 |
| 44 | 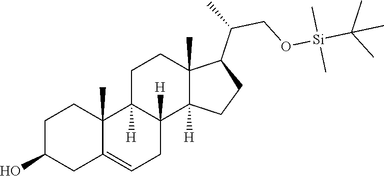 |
| 45 | 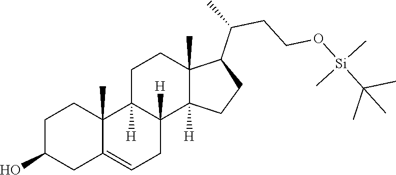 |
| 46 | 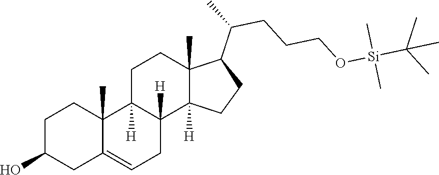 |
| 47 | 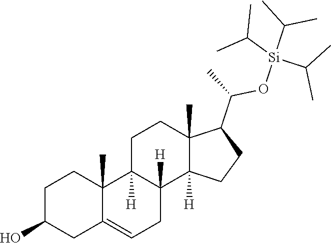 |
| 48 | 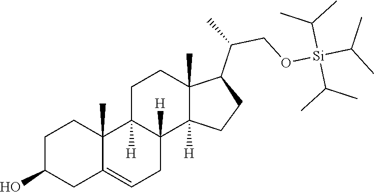 |
| 49 | 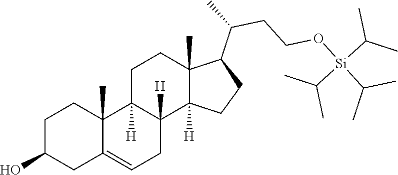 |
| 50 | 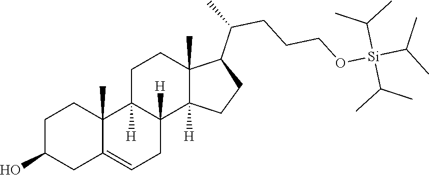 |
| 175 | 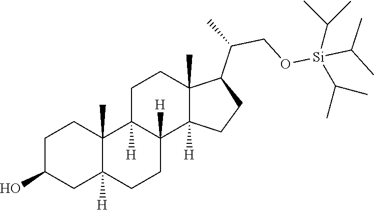 |
| 176 | 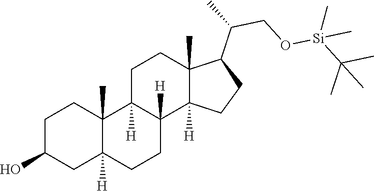 |
| 177 | 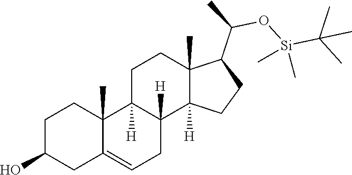 |
| 178 | 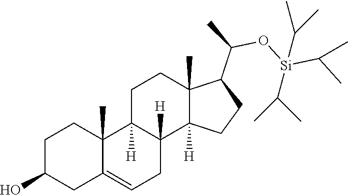 |

In an aspect, the invention features a compound having the structure of any one of compounds S-51-67, S-149 and S-153 in Table 12, or any pharmaceutically acceptable salt thereof.

TABLE 12
Compounds of Formula SIII

| CMPD | |
|---|---|
| No. S- | Structure |
| 51 | 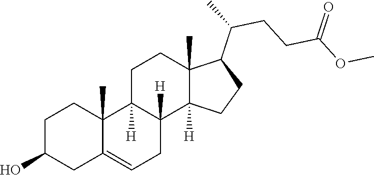 |
| 52 | 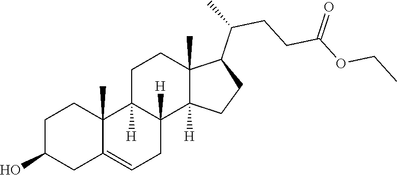 |
| 53 | 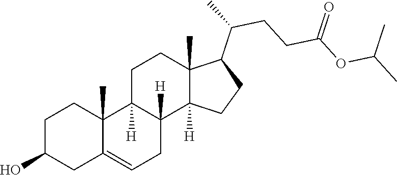 |
| 54 | 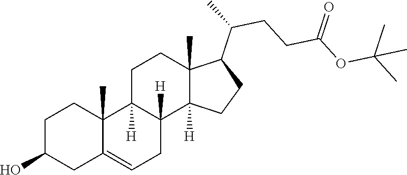 |
| 55 | 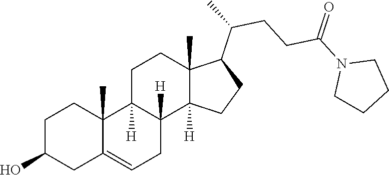 |
| 56 | 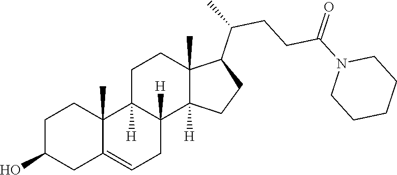 |
| 57 | 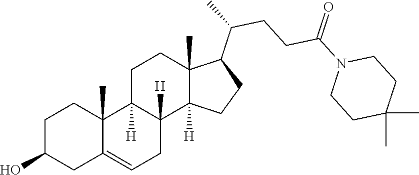 |
| 58 | 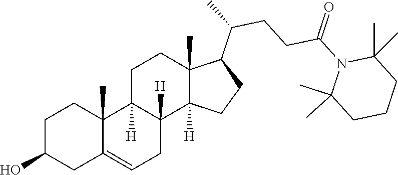 |
| 59 | 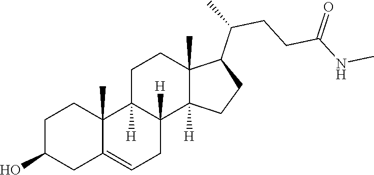 |
| 60 | 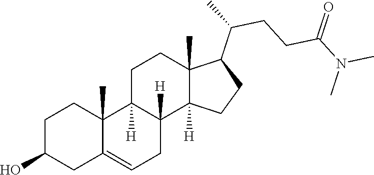 |
| 61 | 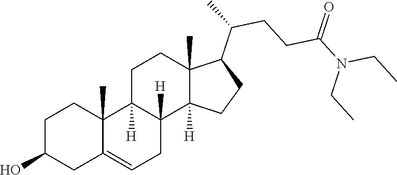 |
| 62 | 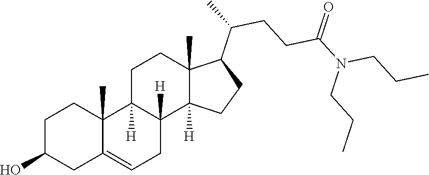 |
| 63 | 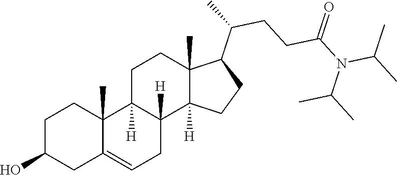 |
| 64 | 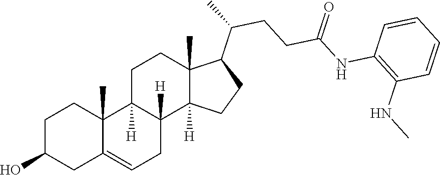 |
| 65 | 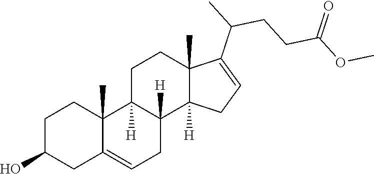 |
| 66 | 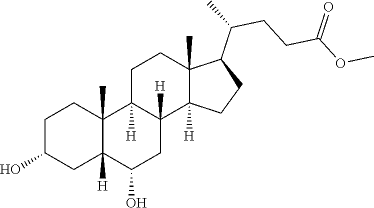 |
| 67 | 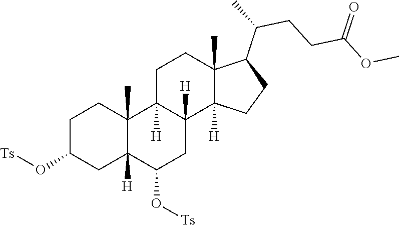 |
| 149 | 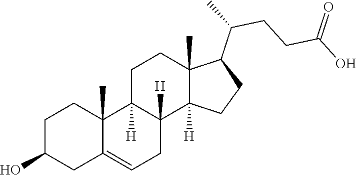 |
| 153 | 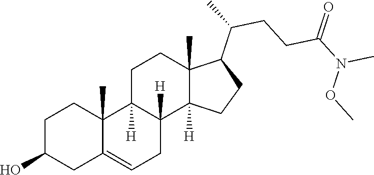 |

In an aspect, the invention features a compound having the structure of any one of compounds S-68-73 in Table 13, or any pharmaceutically acceptable salt thereof.

TABLE 13
Compounds of Formula SIV

| CMPD No. S- | Structure |
|---|---|
| 68 | 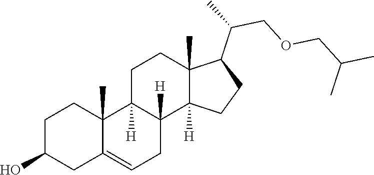 |
| 69 | 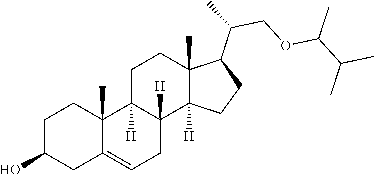 |
| 70 | 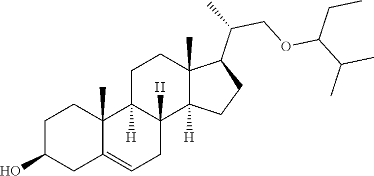 |
| 71 | 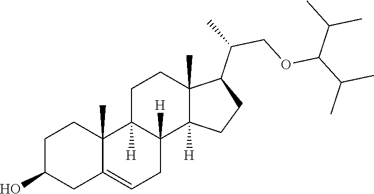 |
| 72 | 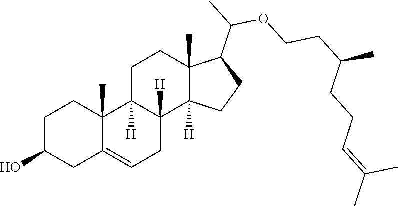 |
| 73 | 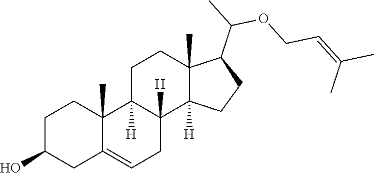 |

In an aspect, the invention features a compound having the structure of any one of compounds S-74-78 in Table 14, or any pharmaceutically acceptable salt thereof.

TABLE 14
Compounds of Formula SV

| CMPD | |
|---|---|
| No. S- | Structure |
| 74 | 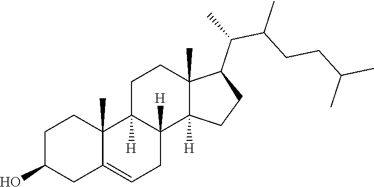 |
| 75 | 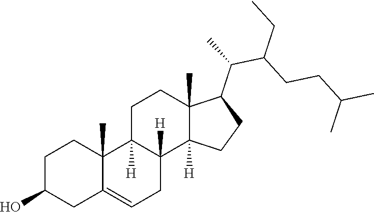 |
| 76 | 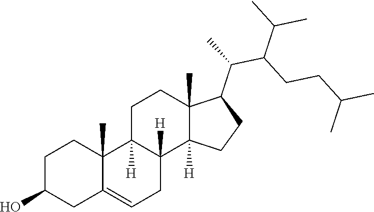 |
| 77 | 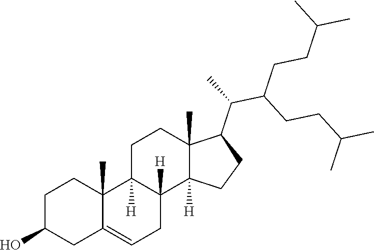 |
| 78 | 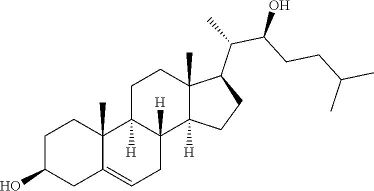 |

In an aspect, the invention features a compound having the structure of any one of compounds S-79 or S-80 in Table 15, or any pharmaceutically acceptable salt thereof.

TABLE 15
Compounds of Formula SVI

| CMPD | |
|---|---|
| No. S- | Structure |
| 79 | 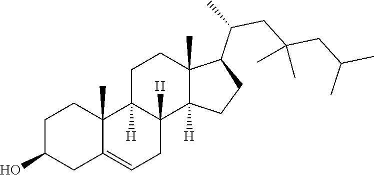 |
| 80 | 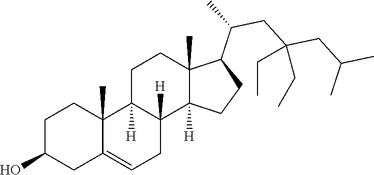 |

In an aspect, the invention features a compound having the structure of any one of compounds S-81-87, S-152 and S-157 in Table 16, or any pharmaceutically acceptable salt thereof.

TABLE 16
Compounds of Formula S-VII

| CMPD | |
|---|---|
| No. S- | Structure |
| 81 | 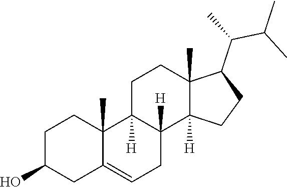 |
| 82 | 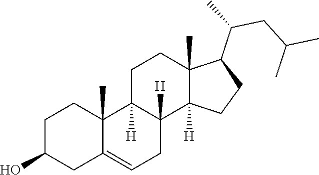 |
| 83 | 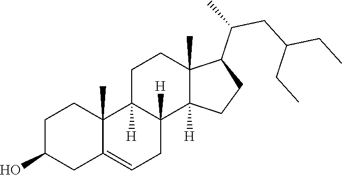 |
| 84 | 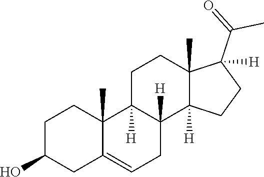 |
| 85 | 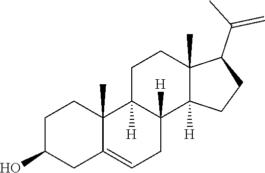 |
| 86 | 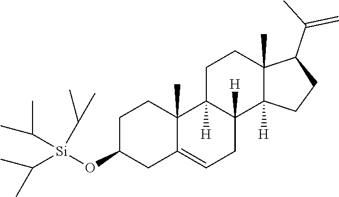 |
| 87 | 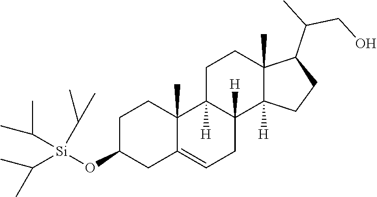 |
| 152 | 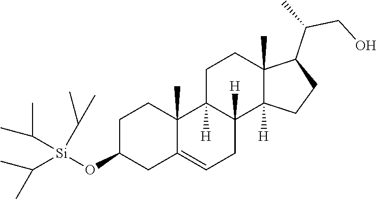 |
| 157 | 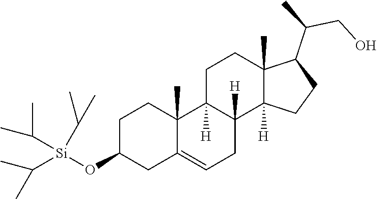 |

In an aspect, the invention features a compound having the structure of any one of compounds S-88-97 in Table 17, or any pharmaceutically acceptable salt thereof.

TABLE 17
Compounds of Formula SVIII

| CMPD | |
|---|---|
| No. S- | Structure |
| 88 | 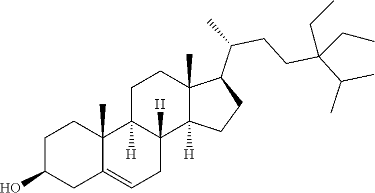 |
| 89 | 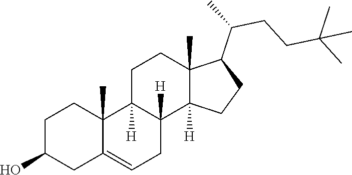 |
| 90 | 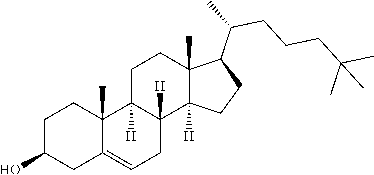 |
| 91 | 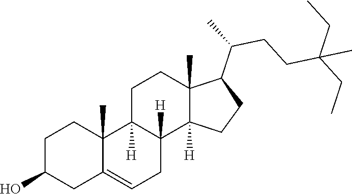 |
| 92 | 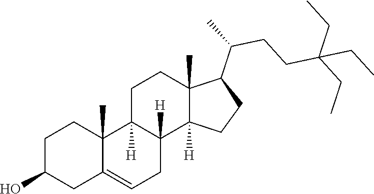 |
| 93 | 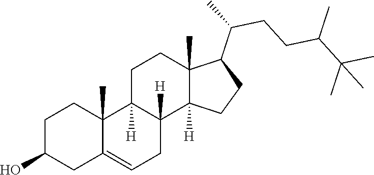 |
| 94 | 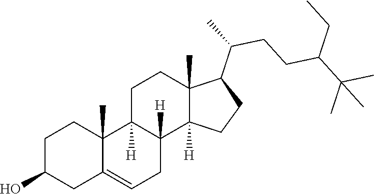 |
| 95 | 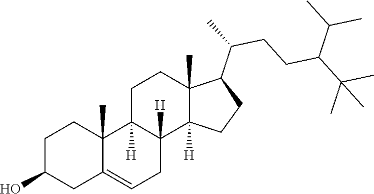 |
| 96 | 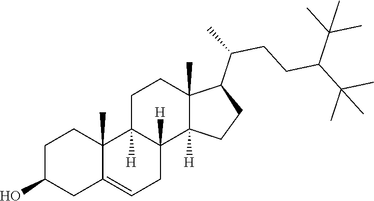 |
| 97 | 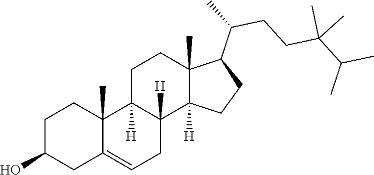 |

In an aspect, the invention features a compound having the structure of any one of compounds S-98-105 and S-180-182 in Table 18, or any pharmaceutically acceptable salt thereof.

TABLE 18
Compounds of Formula SIX

| CMPD | |
|---|---|
| No. S- | Structure |
| 98 | 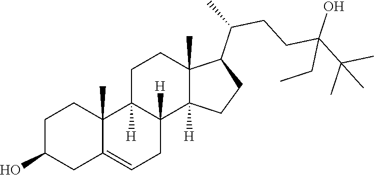 |
| 99 | 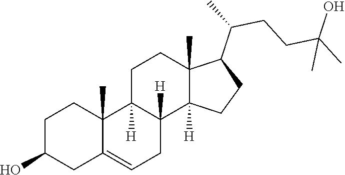 |
| 100 | 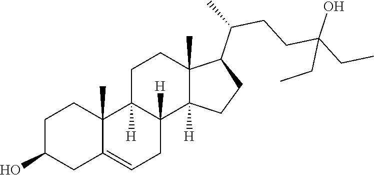 |
| 101 | 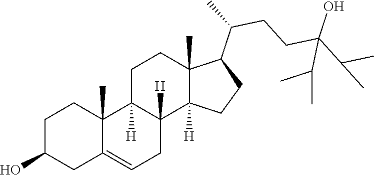 |
| 102 | 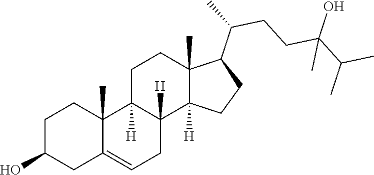 |
| 103 | 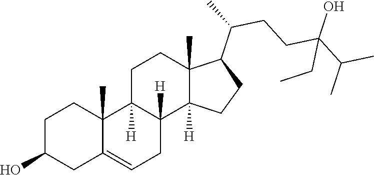 |
| 104 | 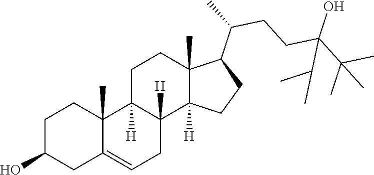 |
| 105 | 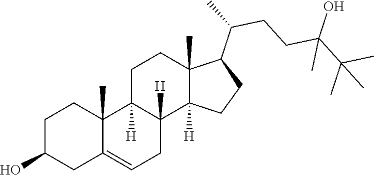 |
| 180 | 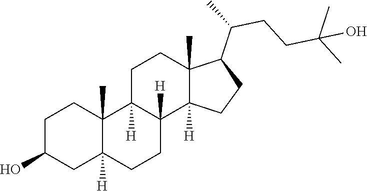 |
| 181 | 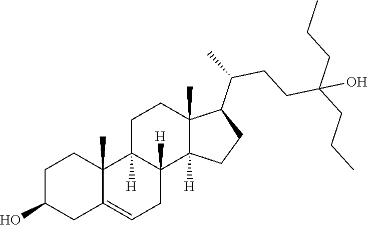 |
| 182 | 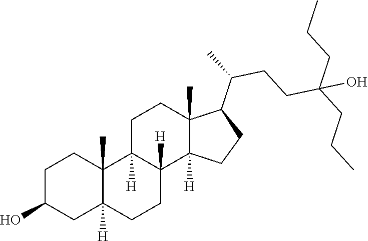 |

In an aspect, the invention features a compound having the structure of compound S-106 in Table 19, or any pharmaceutically acceptable salt thereof.

TABLE 19
Compounds of Formula SX

| CMPD | |
|---|---|
| No. S- | Structure |
| 106 | 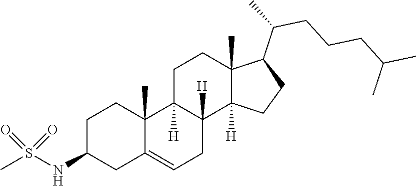 |

In an aspect, the invention features a compound having the structure of compound S-107 or S-108 in Table 20, or any pharmaceutically acceptable salt thereof.

TABLE 20
Compounds of Formula SXI

| CMPD | |
|---|---|
| No. S- | Structure |
| 107 | 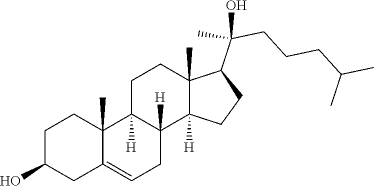 |
| 108 | 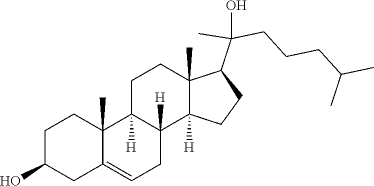 |

In an aspect, the invention features a compound having the structure of compound S-109 in Table 12, or any pharmaceutically acceptable salt thereof.

TABLE 21
Compounds of Formula SXII

| CMPD | |
|---|---|
| No. S- | Structure |
| 109 | 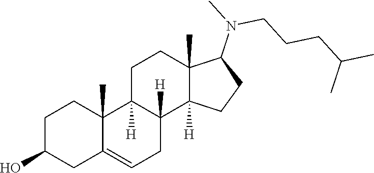 |

In an aspect, the invention features a compound having the structure of any one of compounds S-110-130, S-155, S-156, S-158, S-160, S-161, S-166-168, S-173, S-174 and S-179 in Table 22, or any pharmaceutically acceptable salt thereof.

TABLE 22
Compounds of the Invention

| CMPD | |
|---|---|
| No. S- | Structure |
| 110 | 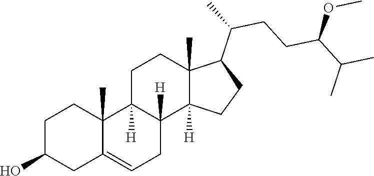 |
| 111 | 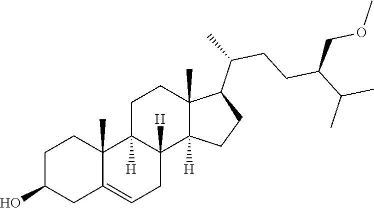 |
| 112 | 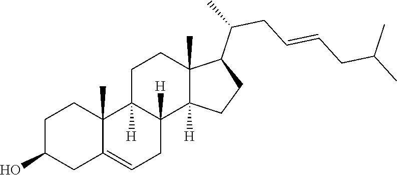 |
| 113 | 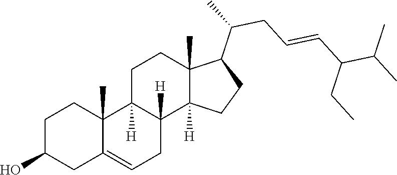 |
| 114 | 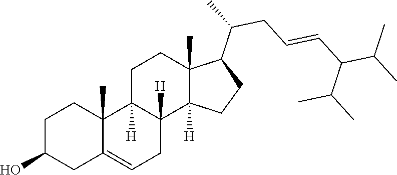 |
| 115 | 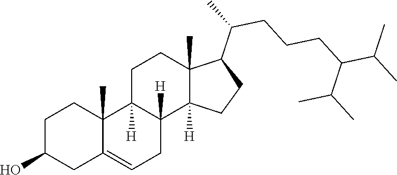 |
| 116 | 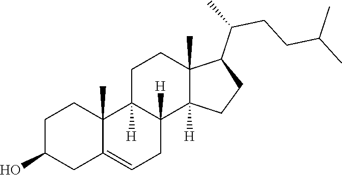 |
| 117 | 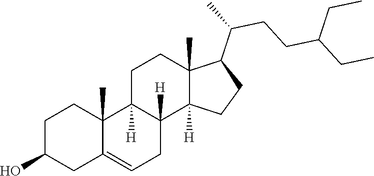 |
| 118 | 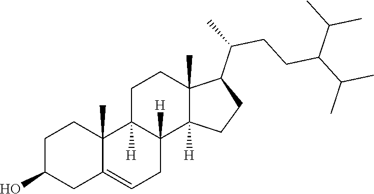 |
| 119 | 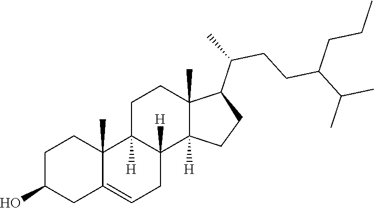 |
| 120 | 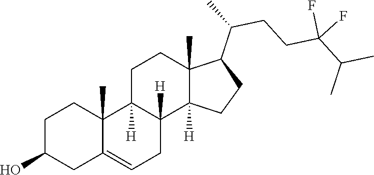 |
| 121 | 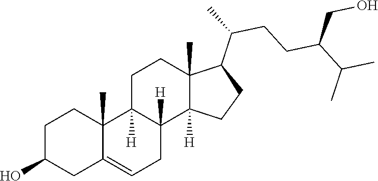 |
| 122 | 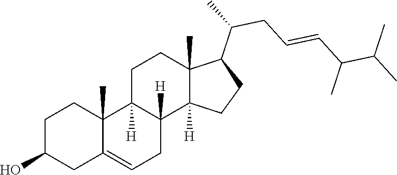 |
| 123 | 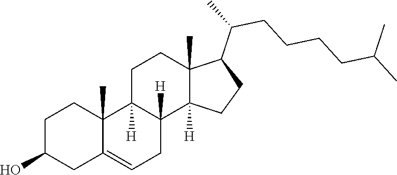 |
| 124 | 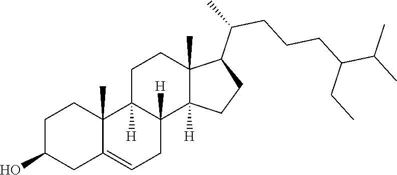 |
| 125 | 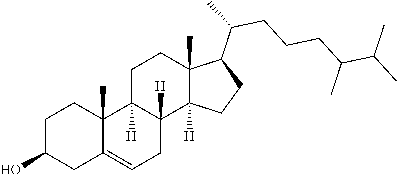 |
| 126 | 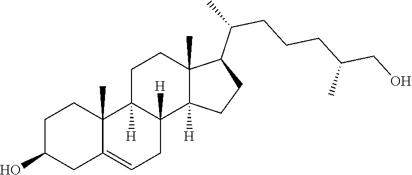 |
| 127 | 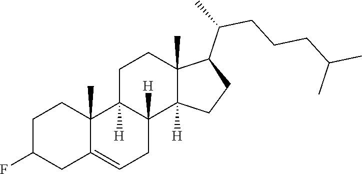 |
| 128 | 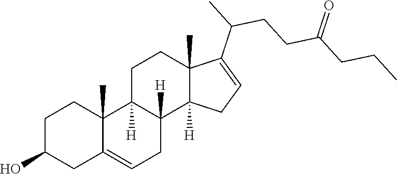 |
| 129 | 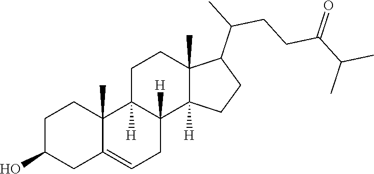 |
| 130 | 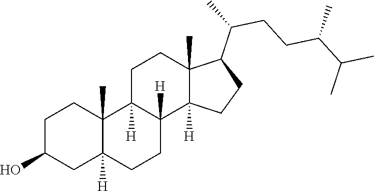 |
| 155 | 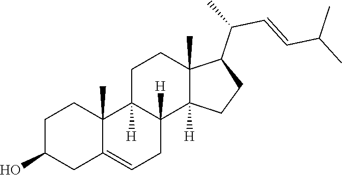 |
| 156 | 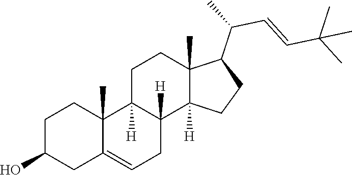 |
| 158 | 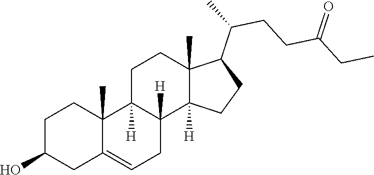 |
| 160 | 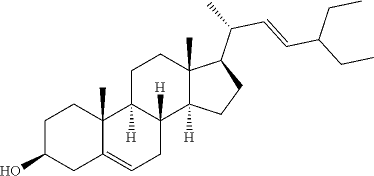 |
| 161 | 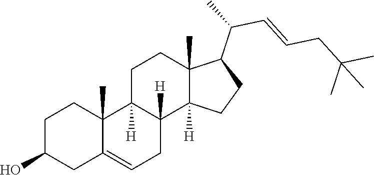 |
| 166 | 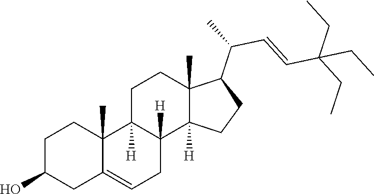 |
| 167 | 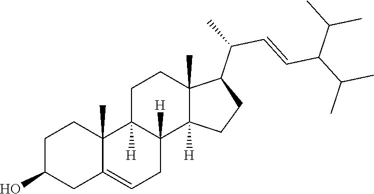 |
| 168 | 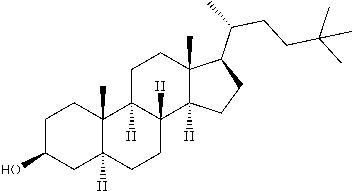 |
| 173 | 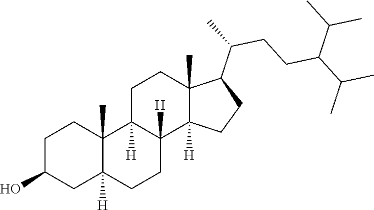 |
| 174 | 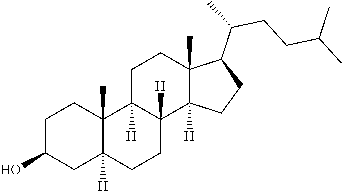 |
| 179 | 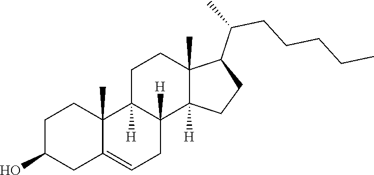 |

In an aspect, the invention features a compound having the structure of any one of compounds S-131-133 in Table 23, or any pharmaceutically acceptable salt thereof.

TABLE 23
Compounds of the Invention

| CMPD | |
|---|---|
| No. S- | Structure |
| 131 | 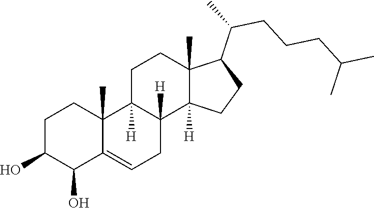 |
| 132 | 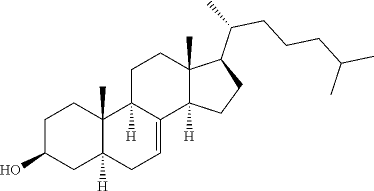 |
| 133 | 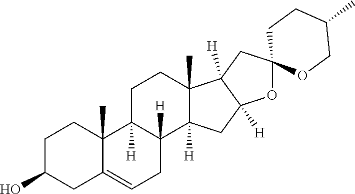 |

In an aspect, the invention features a compound having the structure of any one of compounds S-134-148, S-151 and S-159 in Table 24, or any pharmaceutically acceptable salt thereof.

TABLE 24
Compounds of the Invention

| CMPD | |
|---|---|
| No. S- | Structure |
| 134 | 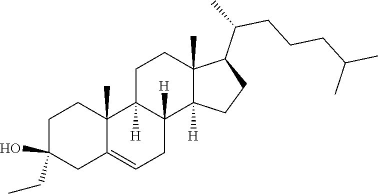 |
| 135 | 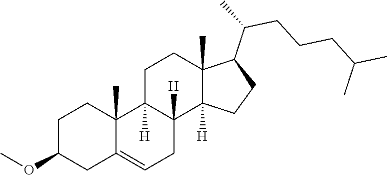 |
| 136 | 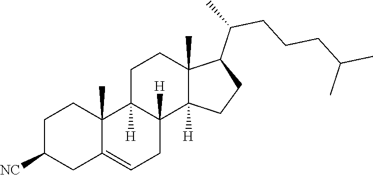 |
| 137 | 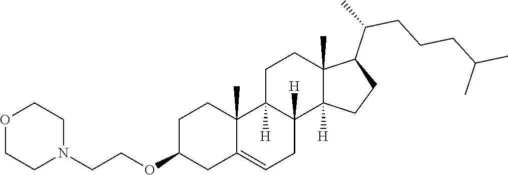 |
| 138 | 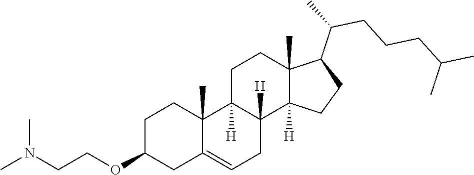 |
| 139 | 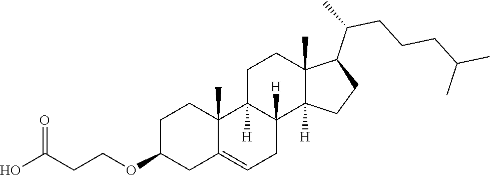 |
| 140 | 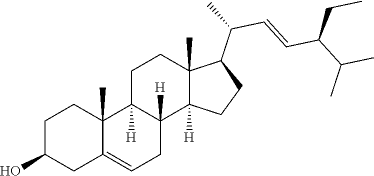 |
| 141 | 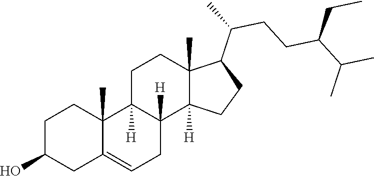 |
| 142 | 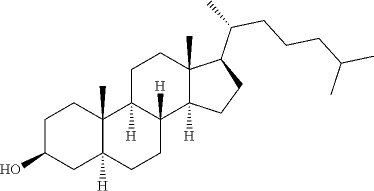 |
| 143 | 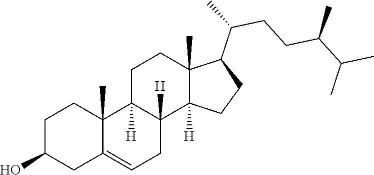 |
| 144 | 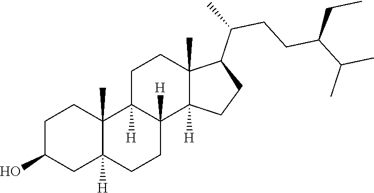 |
| 145 | 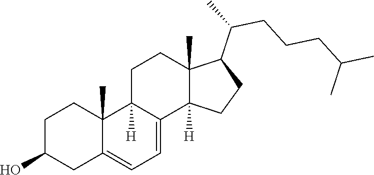 |
| 146 | 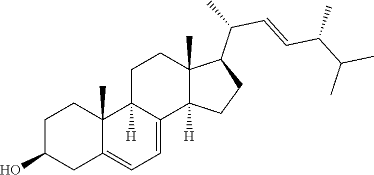 |
| 147 | 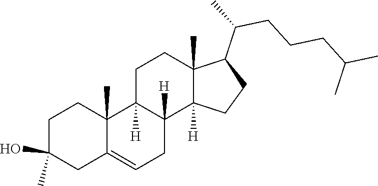 |
| 148 | 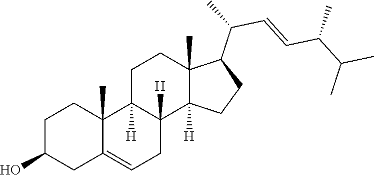 |
| 151 | 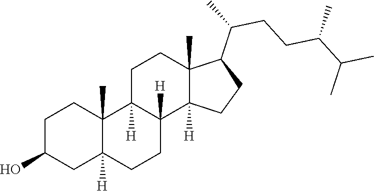 |
| 159 | 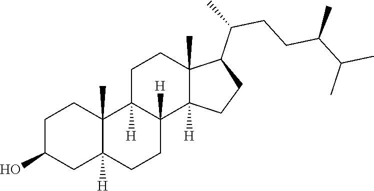 |

The one or more structural lipids of the lipid nanoparticles of the invention can be a composition of structural lipids (e.g., a mixture of two or more structural lipids, a mixture of three or more structural lipids, a mixture of four or more structural lipids, or a mixture of five or more structural lipids). A composition of structural lipids can include, but is not limited to, any combination of sterols (e.g., cholesterol, β-sitosterol, fecosterol, ergosterol, sitosterol, campesterol, stigmasterol, brassicasterol, ergosterol, tomatidine, tomatine, ursolic acid, alpha-tocopherol, or any one of compounds 134-148, 151, and 159 in Table 15). For example, the one

Or more structural lipids of the lipid nanoparticles of the invention can be composition 183 in Table 25.

TABLE 25
Structural Lipid Compositions

| Compo- | |
|---|---|
| sition | |
| S- No. | Structure |
| 183 | 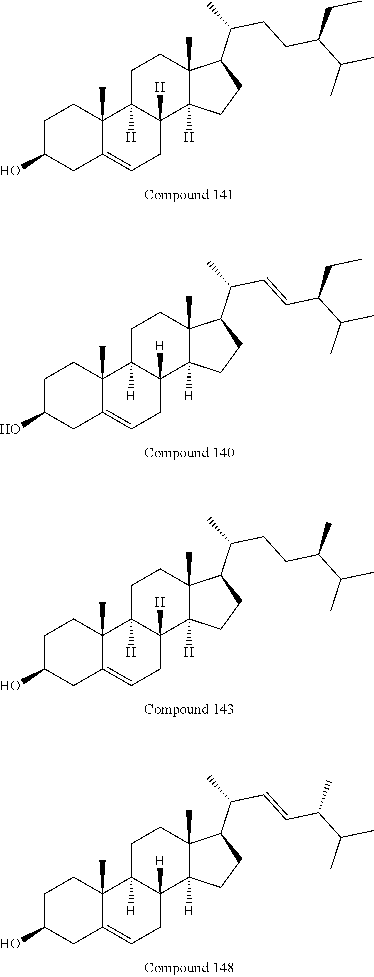 |

Composition S-183 is a mixture of compounds S-141, S-140, S-143, and S-148. In some embodiments, composition S-183 includes about 35% to about 45% of compound S-141, about 20% to about 30% of compound S-140, about 20% to about 30% compound S-143, and about 5% to about 15% of compound S-148. In some embodiments, composition 183 includes about 40% of compound S-141, about 25% of compound S-140, about 25% compound S-143, and about 10% of compound S-148.

In some embodiments, the structural lipid is a pytosterol. In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, Δ5-avenaserol, Δ7-avenaserol or a Δ7-stigmasterol, including analogs, salts or esters thereof, alone or in combination. In some embodiments, the phytosterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol.

### Ratio of Compounds

A lipid nanoparticle of the invention can include a structural component as described herein. The structural component of the lipid nanoparticle can be any one of compounds S-1-148, a mixture of one or more structural compounds of the invention and/or any one of compounds S-1-148 combined with a cholesterol and/or a phytosterol.

For example, the structural component of the lipid nanoparticle can be a mixture of one or more structural compounds (e.g. any of Compounds S-1-148) of the invention with cholesterol. The mol % of the structural compound present in the lipid nanoparticle relative to cholesterol can be from 0-99 mol %. The mol % of the structural compound present in the lipid nanoparticle relative to cholesterol can be about 10 mol %, 20 mol %, 30 mol %, 40 mol %, 50 mol %, 60 mol %, 70 mol %, 80 mol %, or 90 mol %.

In one aspect, the invention features a composition including two or more sterols, wherein the two or more sterols include at least two of: β-sitosterol, sitostanol, camesterol, stigmasterol, and brassicasteol. The composition may additionally comprise cholesterol. In one embodiment, β-sitosterol comprises about 35-99%, e.g., about 40%, 50%, 60%, 70%, 80%, 90%, 95% or greater of the non-cholesterol sterol in the composition.

In another aspect, the invention features a composition including two or more sterols, wherein the two or more sterols include β-sitosterol and campesterol, wherein β-sitosterol includes 95-99.9% of the sterols in the composition and campesterol includes 0.1-5% of the sterols in the composition.

In some embodiments, the composition further includes sitostanol. In some embodiments, β-sitosterol includes 95-99.9%, campesterol includes 0.05-4.95%, and sitostanol includes 0.05-4.95% of the sterols in the composition.

In another aspect, the invention features a composition including two or more sterols, wherein the two or more sterols include β-sitosterol and sitostanol, wherein β-sitosterol includes 95-99.9% of the sterols in the composition and sitostanol includes 0.1-5% of the sterols in the composition.

In some embodiments, the composition further includes campesterol. In some embodiments, β-sitosterol includes 95-99.9%, campesterol includes 0.05-4.95%, and sitostanol includes 0.05-4.95% of the sterols in the composition.

In some embodiments, the composition further includes campesterol. In some embodiments, β-sitosterol includes 75-80%, campesterol includes 5-10%, and sitostanol includes 10-15% of the sterols in the composition.

In some embodiments, the composition further includes an additional sterol. In some embodiments, β-sitosterol includes 35-45%, stigmasterol includes 20-30%, and campesterol includes 20-30%, and brassicasterol includes 1-5% of the sterols in the composition.

In another aspect, the invention features a composition including a plurality of lipid nanoparticles, wherein the plurality of lipid nanoparticles include an ionizable lipid and two or more sterols, wherein the two or more sterols include β-sitosterol, and campesterol and β-sitosterol includes 95-99.9% of the sterols in the composition and campesterol includes 0.1-5% of the sterols in the composition.

In some embodiments, the two or more sterols further includes sitostanol. In some embodiments, β-sitosterol includes 95-99.9%, campesterol includes 0.05-4.95%, and sitostanol includes 0.05-4.95% of the sterols in the composition.

In another aspect, the invention features a composition including a plurality of lipid nanoparticles, wherein the plurality of lipid nanoparticles include an ionizable lipid and two or more sterols, wherein the two or more sterols include β-sitosterol, and sitostanol and β-sitosterol includes 95-99.9% of the sterols in the composition and sitostanol includes 0.1-5% of the sterols in the composition.

In some embodiments, the two or more sterols further includes campesterol. In some embodiments, β-sitosterol includes 95-99.9%, campesterol includes 0.05-4.95%, and sitostanol includes 0.05-4.95% of the sterols in the composition.

(iii) Non-Cationic Helper Lipids/Phospholipids

In some embodiments, the lipid-based composition (e.g., LNP) described herein comprises one or more non-cationic helper lipids. In some embodiments, the non-cationic helper lipid is a phospholipid. In some embodiments, the non-cationic helper lipid is a phospholipid substitute or replacement.

As used herein, the term “non-cationic helper lipid” refers to a lipid comprising at least one fatty acid chain of at least 8 carbons in length and at least one polar head group moiety. In one embodiment, the helper lipid is not a phosphatidyl choline (PC). In one embodiment the non-cationic helper lipid is a phospholipid or a phospholipid substitute. In some embodiments, the phospholipid or phospholipid substitute can be, for example, one or more saturated or (poly)unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the non-cationic helper lipid is a DSPC analog, a DSPC substitute, oleic acid, or an oleic acid analog.

In some embodiments, a non-cationic helper lipid is a non-phosphatidyl choline (PC) zwitterionic lipid, a DSPC analog, oleic acid, an oleic acid analog, or a 1,2-distearoyl-i77-glycero-3-phosphocholine (DSPC) substitute.

Phospholipids

The lipid composition of the pharmaceutical composition disclosed herein can comprise one or more non-cationic helper lipids. In some embodiments, the non-cationic helper lipids are phospholipids, for example, one or more saturated or (poly)unsaturated phospholipids or a combination thereof. In general, phospholipids comprise a phospholipid moiety and one or more fatty acid moieties. As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). A phospholipid or an analog or derivative thereof may include choline. A phospholipid or an analog or derivative thereof may not include choline. Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

The lipid component of a lipid nanoparticle of the disclosure may include one or more phospholipids, such as one or more (poly)unsaturated lipids. Phospholipids may assemble into one or more lipid bilayers. In general, phospholipids may include a phospholipid moiety and one or more fatty acid moieties. For example, a phospholipid may be a lipid according to Formula (H III):

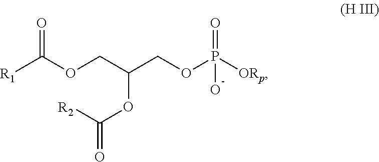

in which Rₚ represents a phospholipid moiety and R₁ and R₂ represent fatty acid moieties with or without unsaturation that may be the same or different. A phospholipid moiety may be selected from the non-limiting group consisting of phosphatidylcholine, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a LNP to facilitate membrane permeation or cellular recognition or in conjugating a LNP to a useful component such as a targeting or imaging moiety (e.g., a dye). Each possibility represents a separate embodiment of the present invention.

Phospholipids useful in the compositions and methods described herein may be selected from the non-limiting group consisting of 1,2-distearoyl-sn-glycero-3-phosphocholine (DSPC),
1,2-dioleoyl-sn-glycero-3-phosphoethanolamine (DOPE),
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine (18:0 Diether PC),
1-oleoyl-2-cholesterylhemisuccinoyl-sn-glycero-3-phosphocholine (OChemsPC),
1-hexadecyl-sn-glycero-3-phosphocholine (C16 Lyso PC),
1,2-dilinolenoyl-sn-glycero-3-phosphocholine (18:3 (cis) PC),
1,2-diarachidonoyl-sn-glycero-3-phosphocholine (DAPC),
1,2-didocosahexaenoyl-sn-glycero-3-phosphocholine (22:6 (cis) PC)
1,2-diphytanoyl-sn-glycero-3-phosphoethanolamine (4ME 16.0 PE),
1,2-distearoyl-sn-glycero-3-phosphoethanolamine (DSPE),
1,2-dilinoleoyl-sn-glycero-3-phosphoethanolamine (PE (18:2/18:2),
1,2-dilinolenoyl-sn-glycero-3-phosphoethanolamine (PE 18:3(9Z, 12Z, 15Z),
1,2-diarachidonoyl-sn-glycero-3-phosphoethanolamine (DAPE 18:3 (9Z, 12Z, 15Z),
1,2-didocosahexaenoyl-sn-glycero-3-phosphoethanolamine (22:6 (cis) PE),
1,2-dioleoyl-sn-glycero-3-phospho-rac-(1-glycerol) sodium salt (DOPG), and sphingomyelin. Each possibility represents a separate embodiment of the invention.

In some embodiments, a LNP includes DSPC. In certain embodiments, a LNP includes DOPE. In some embodiments, a LNP includes DMPE. In some embodiments, a LNP includes both DSPC and DOPE.

In one embodiment, a non-cationic helper lipid for use in an immune cell delivery LNP is selected from the group consisting of: DSPC, DMPE, and DOPC or combinations thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

Examples of phospholipids include, but are not limited to, the following:

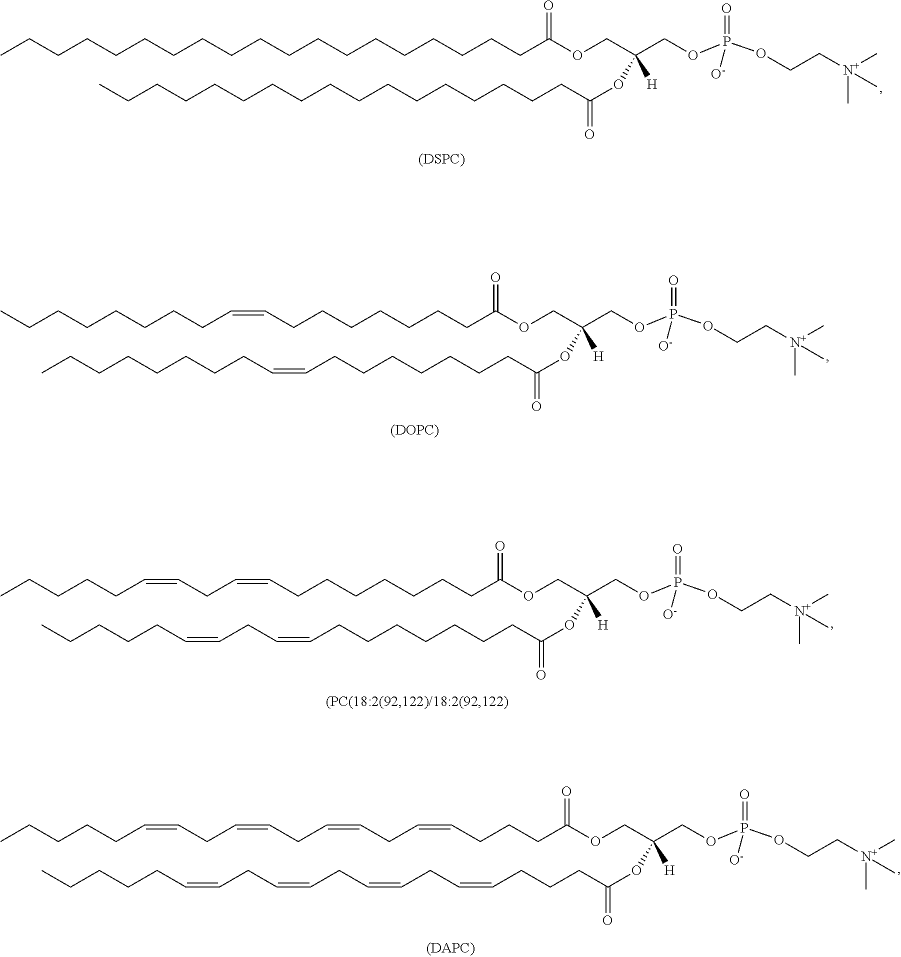

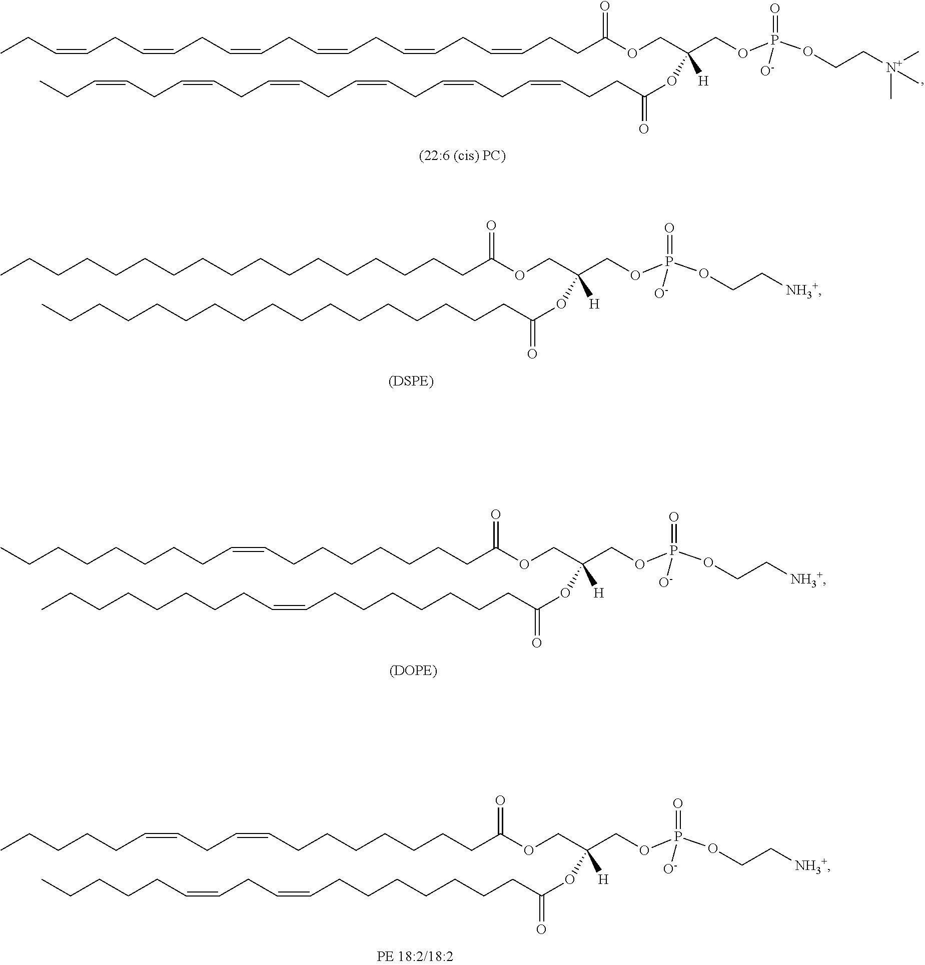

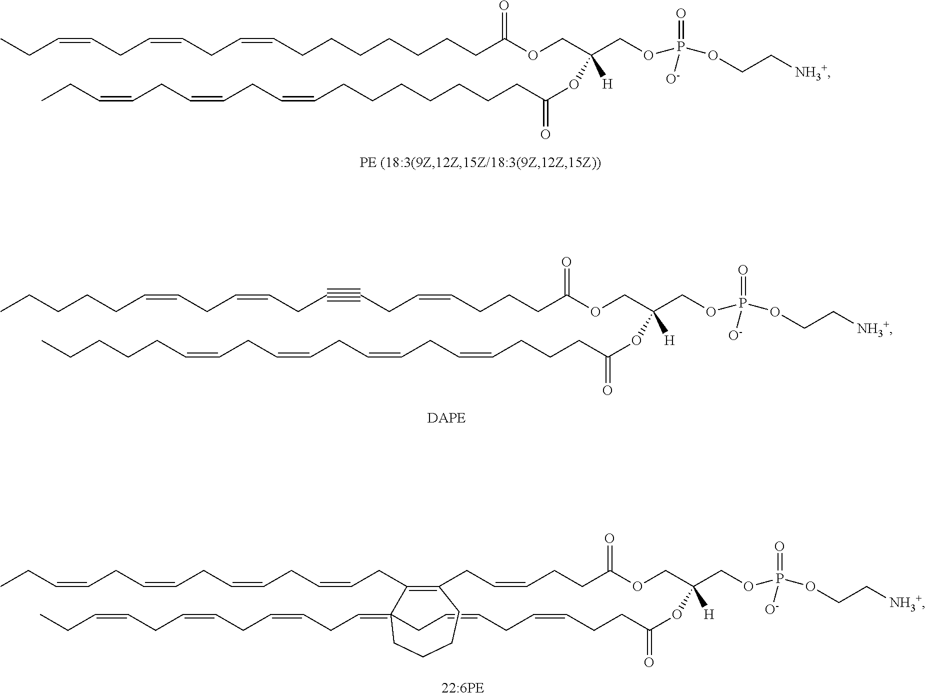

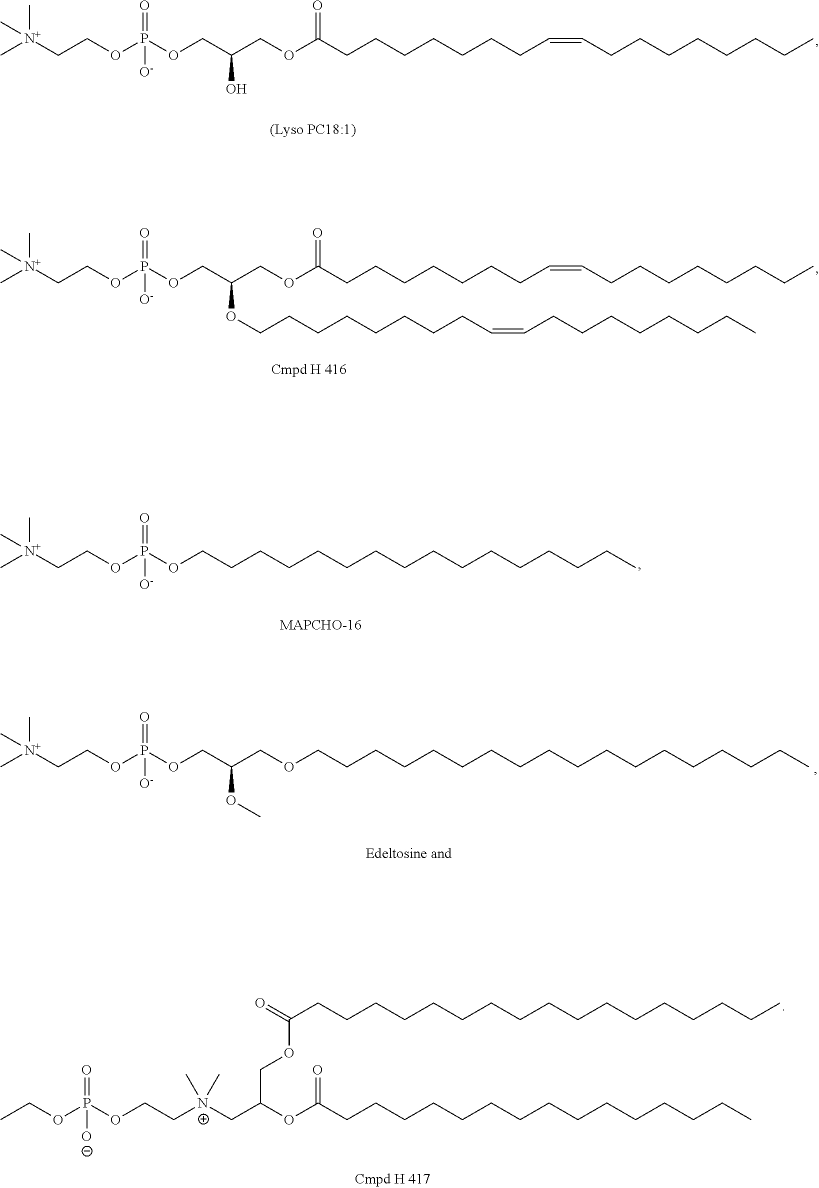

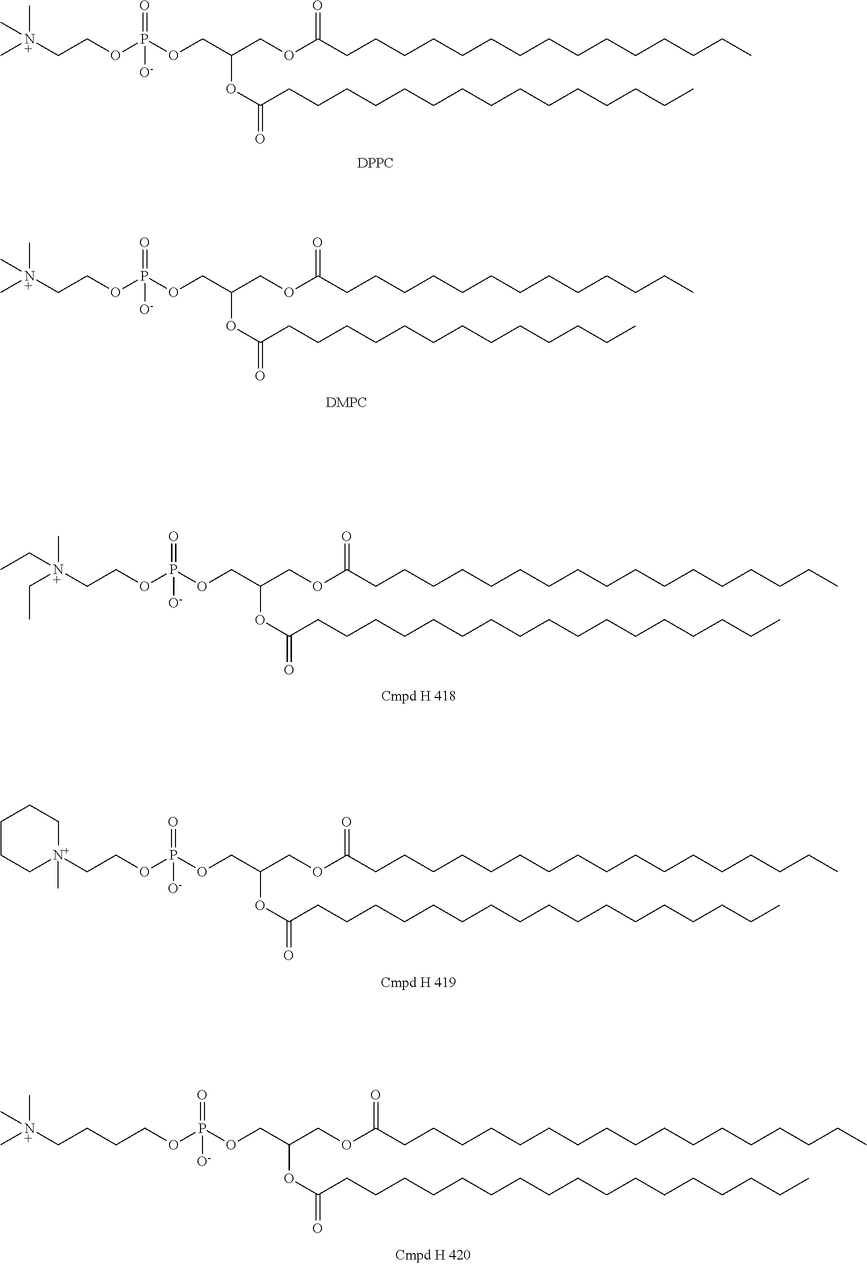

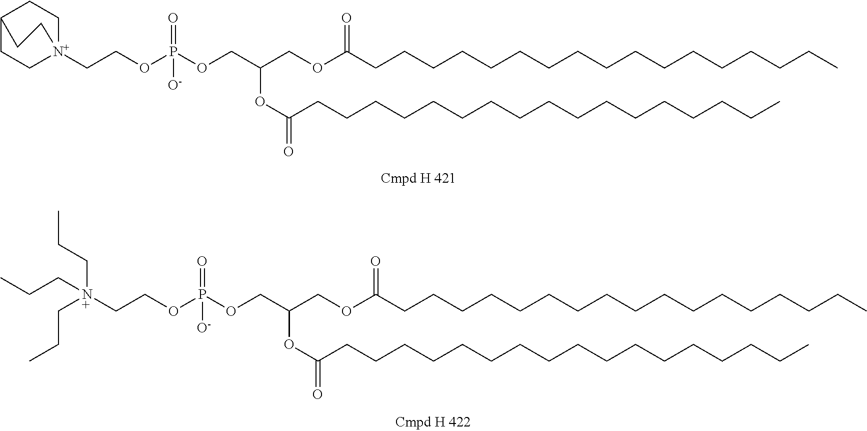

In certain embodiments, a phospholipid useful or potentially useful in the present invention is an analog or variant of DSPC (1,2-dioctadecanoyl-sn-glycero-3-phosphocholine). In certain embodiments, a phospholipid useful or potentially useful in the present invention is a compound of Formula (H IX):

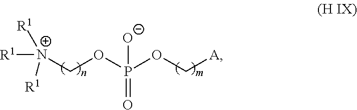

or a salt thereof, wherein:

each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

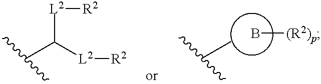

each instance of L² is independently a bond or optionally substituted C₁₋₆ alkylene, wherein one methylene unit of the optionally substituted C₁-6 alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

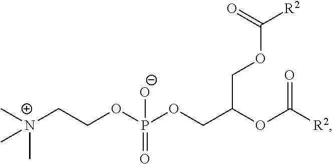

wherein each instance of R² is independently unsubstituted alkyl, unsubstituted alkenyl, or unsubstituted alkynyl.

i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IX), at least one of R¹ is not methyl. In certain embodiments, at least one of R¹ is not hydrogen or methyl. In certain embodiments, the compound of Formula (IX) is of one of the following formulae:

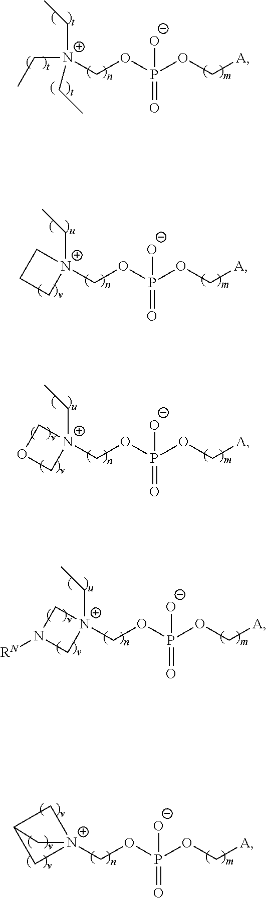

or a salt thereof, wherein:

each t is independently 1, 2, 3, 4, 5, 6, 7, 8, 9, or 10;

each u is independently 0, 1, 2, 3, 4, 5, 6, 7, 8, 9, or 10; and

each v is independently 1, 2, or 3.

In certain embodiments, the compound of Formula (H IX) is of one of the following formulae:

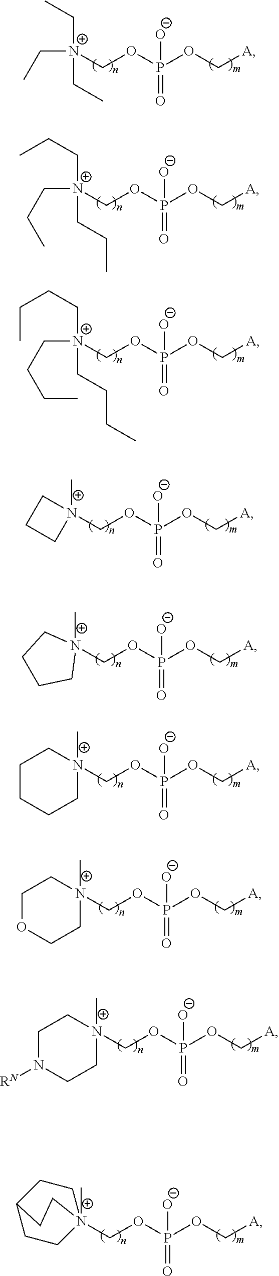

or a salt thereof.

In certain embodiments, a compound of Formula (H IX) is one of the following:

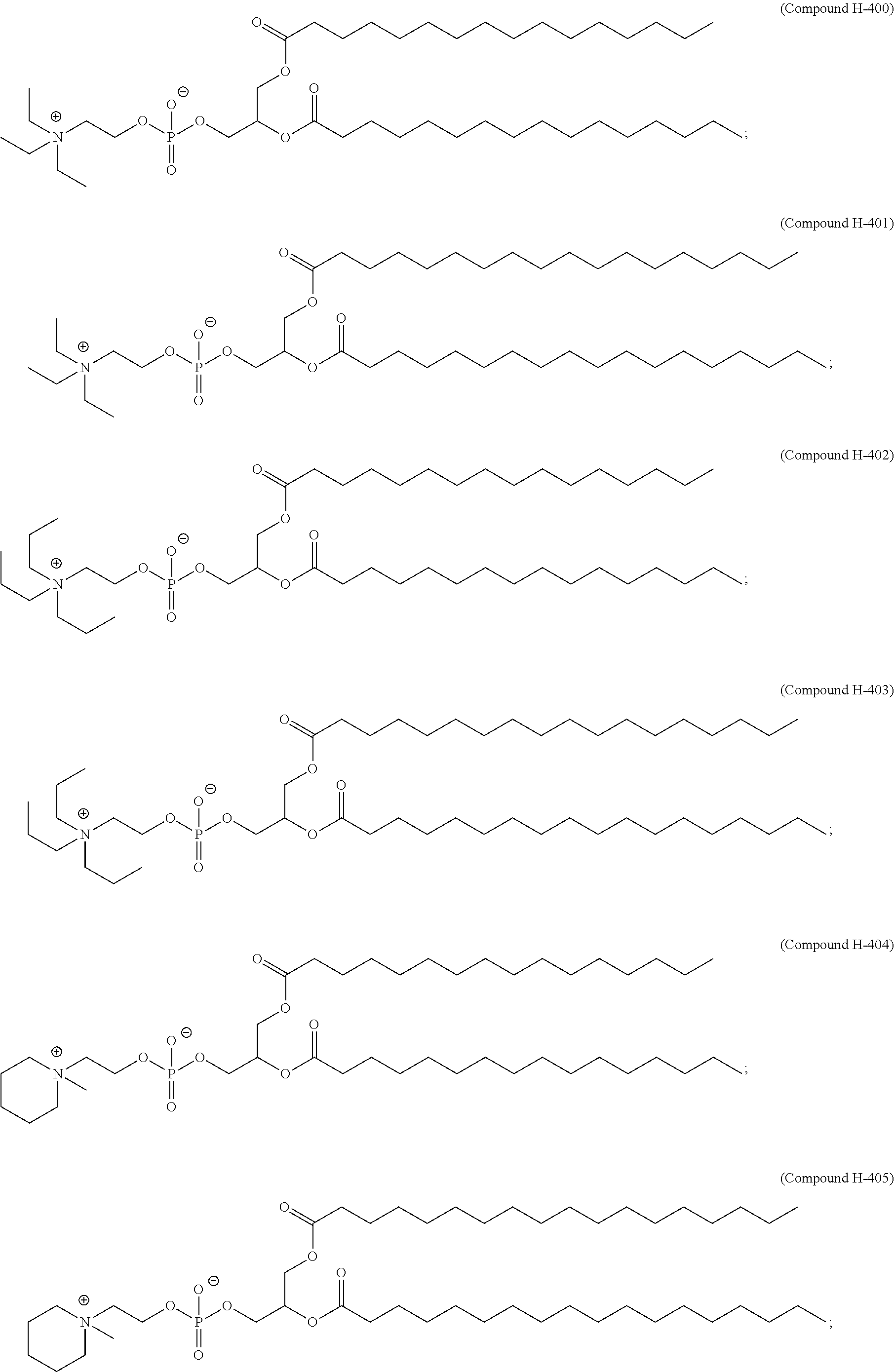

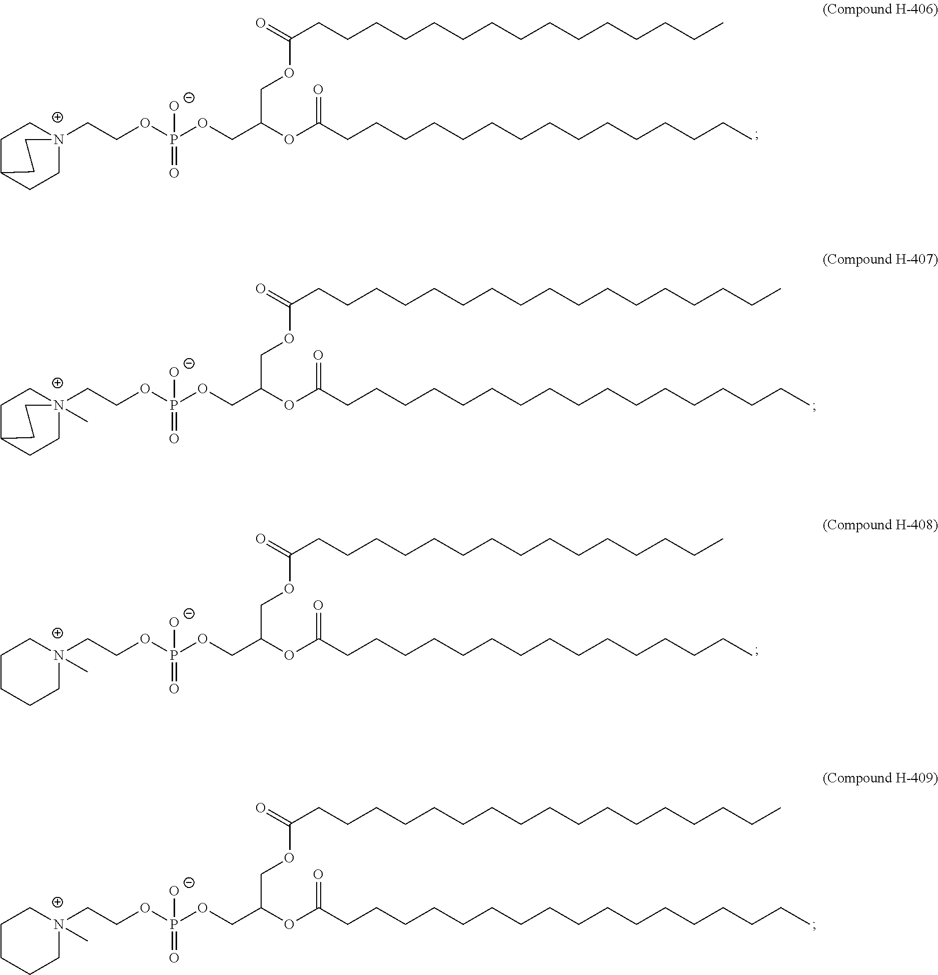

or a salt thereof.

In one embodiment, an immune cell delivery LNP comprises Compound H-409 as a non-cationic helper lipid.

(ii) Phospholipid Tail Modifications

In certain embodiments, a phospholipid useful or potentially useful in the present invention comprises a modified tail. In certain embodiments, a phospholipid useful or potentially useful in the present invention is DSPC (1,2-dioctadecanoyl-sn-glycero-3-phosphocholine), or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H IX) is of Formula (H IX-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (H IX) is of Formula (H IX-c):

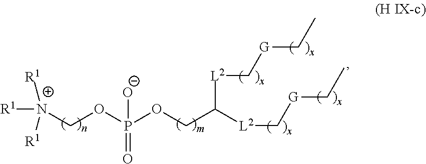

or a salt thereof, wherein:
each x is independently an integer between 0-30, inclusive; and

each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (H IX-c) is of Formula (H IX-c-1):

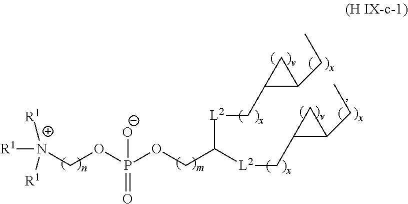

or salt thereof, wherein:
each instance of v is independently 1, 2, or 3.

In certain embodiments, the compound of Formula (H IX-c) is of Formula (H IX-c-2):

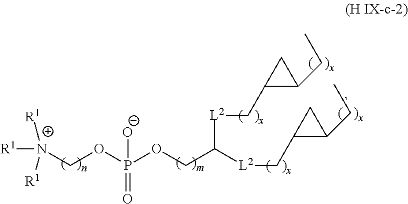

or a salt thereof.

In certain embodiments, the compound of Formula (IX-c) is of the following formula:

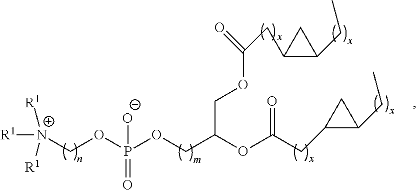

or a salt thereof.

In certain embodiments, the compound of Formula (H IX-c) is the following:

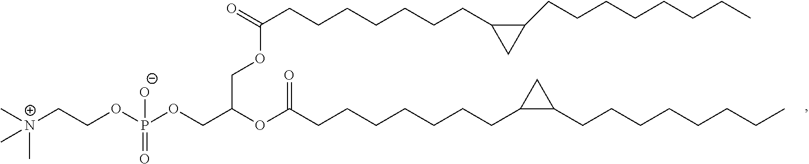 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H IX-c) is of Formula (H IX-c-3):

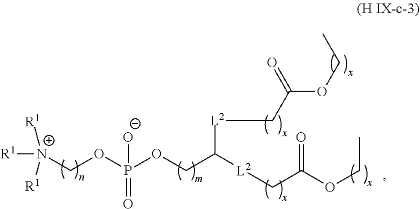

or a salt thereof.

In certain embodiments, the compound of Formula (H IX-c) is of the following formulae:

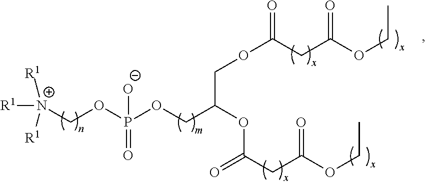

or a salt thereof.

In certain embodiments, the compound of Formula (H IX-c) is the following:

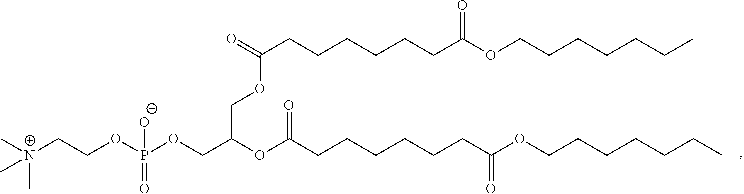 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (H IX), wherein n is 1, 3, 4, 5, 6, 7, 8, 9, or 10. For example, in certain embodiments, a compound of Formula (H IX) is of one of the following formulae:

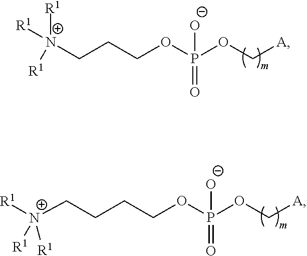

or a salt thereof.

In certain embodiments, a compound of Formula (H IX) is one of the following:

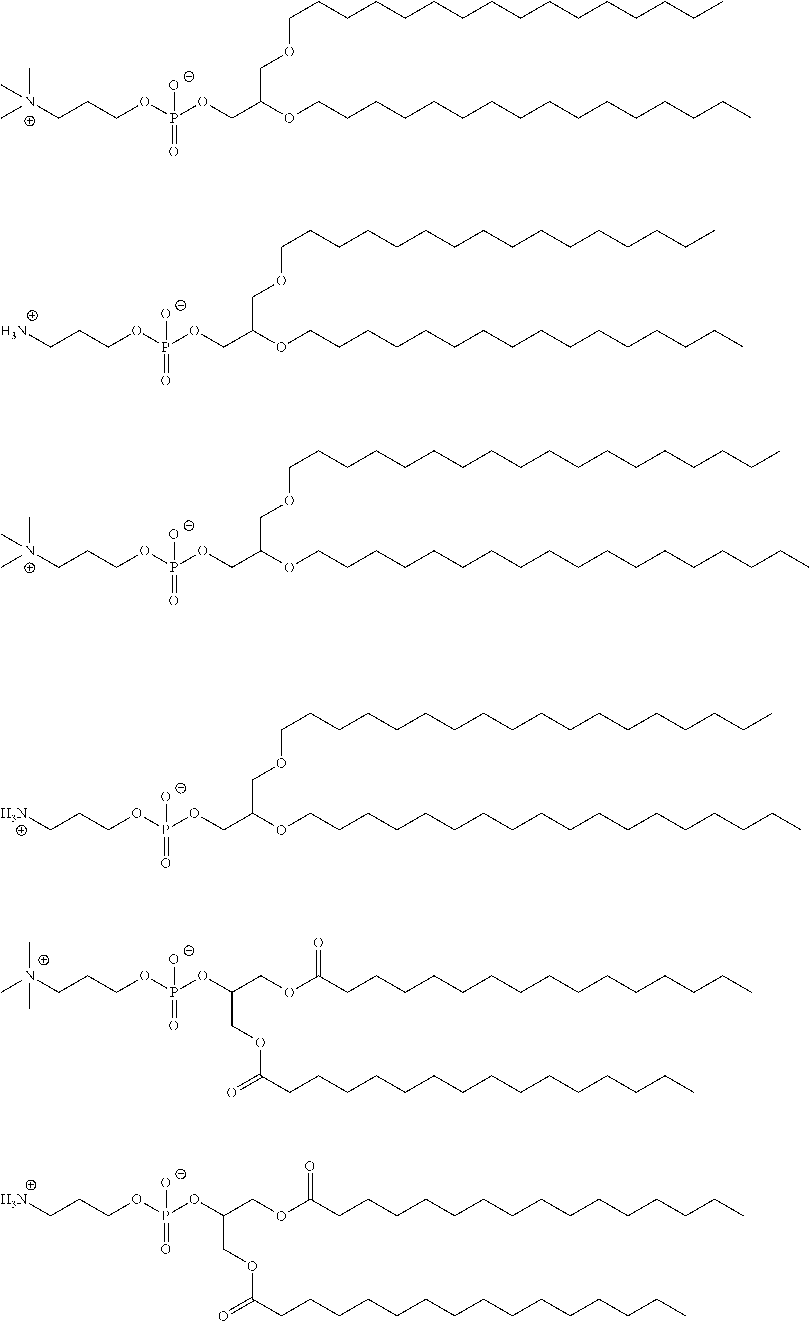

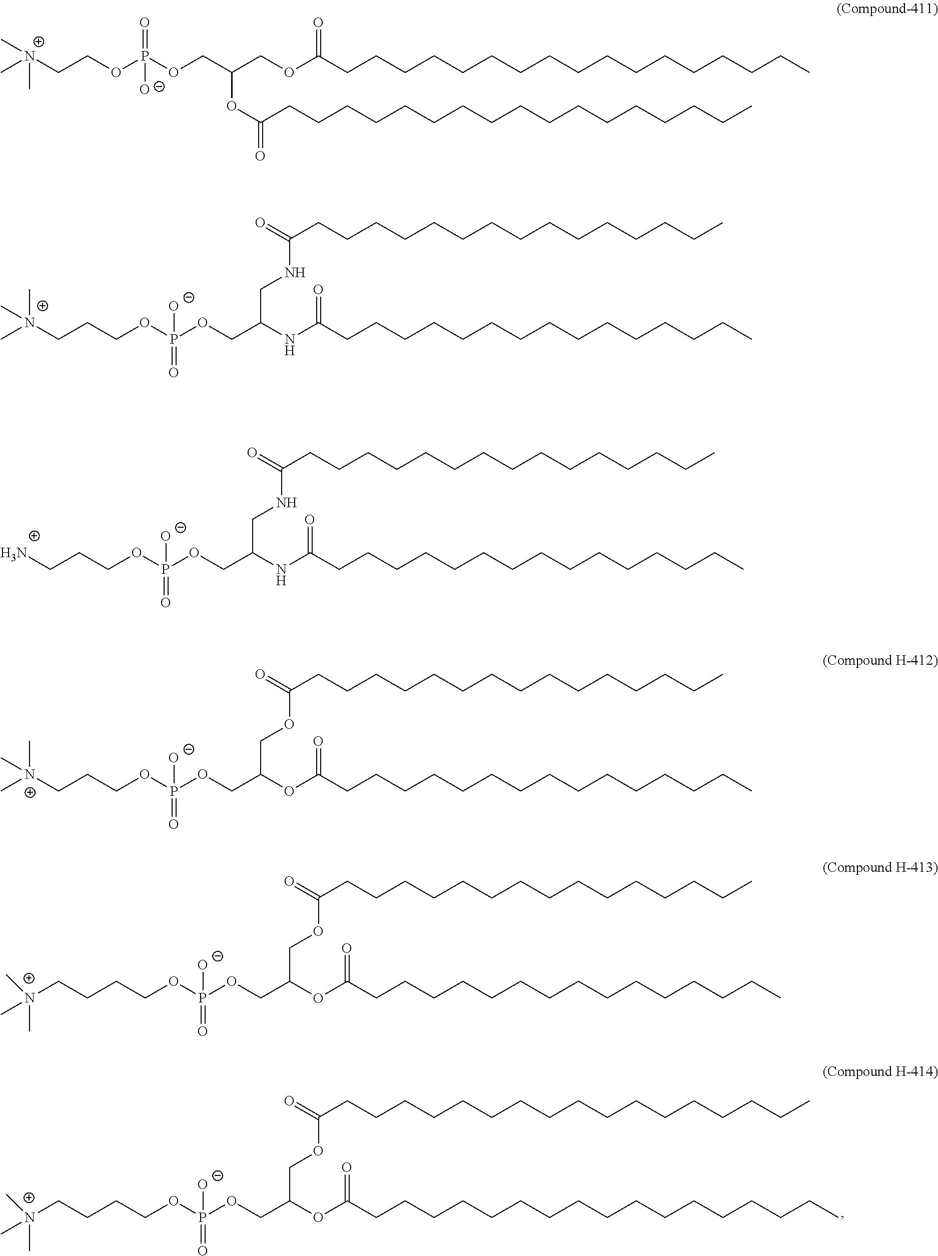

or salts thereof.

In certain embodiments, an alternative lipid is used in place of a phospholipid of the invention. Non-limiting examples of such alternative lipids include the following:

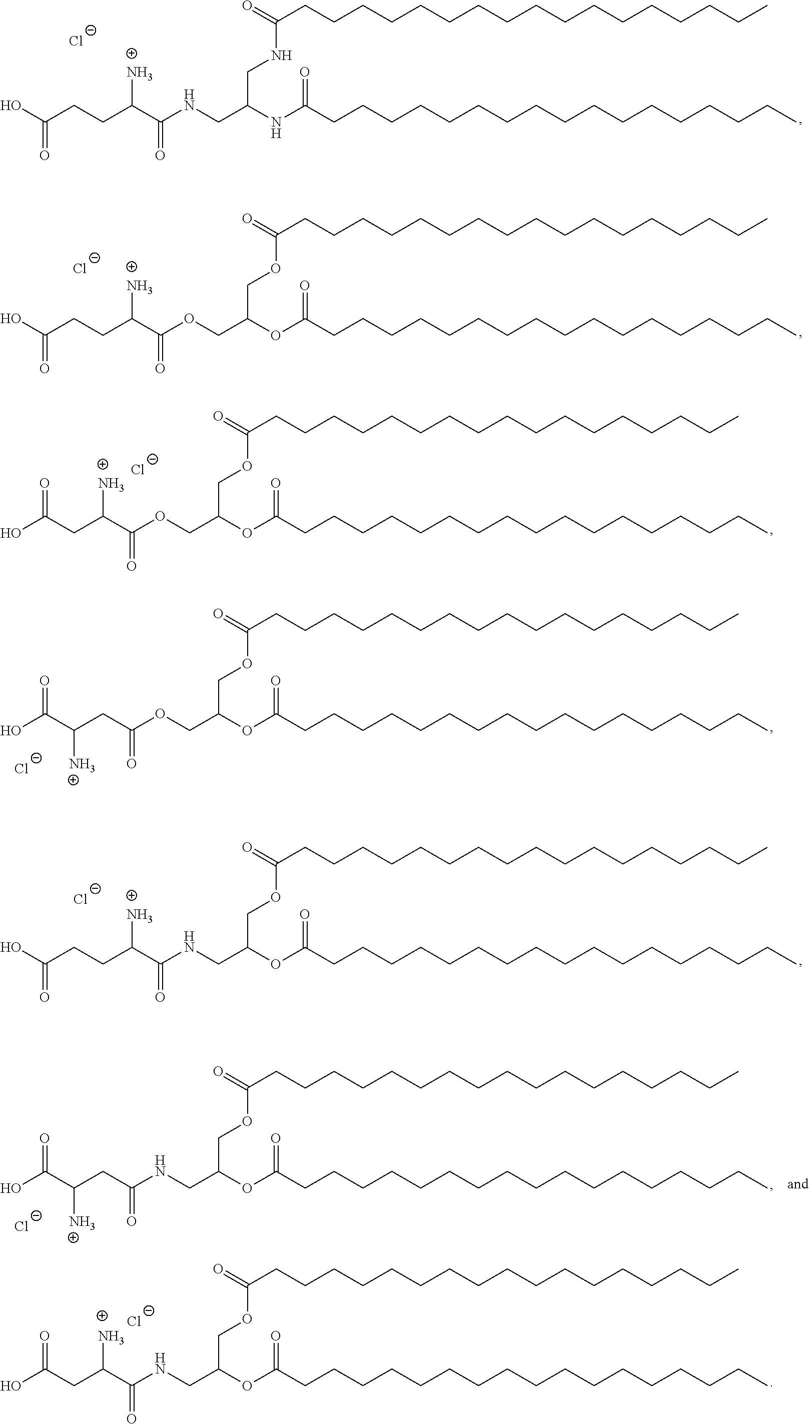

### Phospholipid Tail Modifications

In certain embodiments, a phospholipid useful in the present invention comprises a modified tail. In certain embodiments, a phospholipid useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H I) is of Formula (H I-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N}), —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —SO(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (H I-a) is of Formula (H I-c):

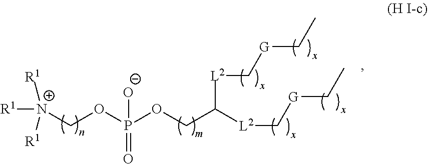

or a salt thereof, wherein:

each x is independently an integer between 0-30, inclusive; and

each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (H I-c) is of Formula (H I-c-1):

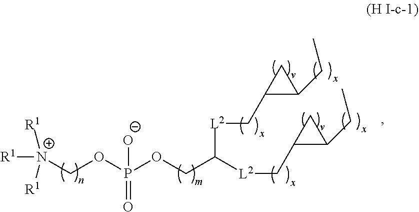

or salt thereof, wherein:

each instance of v is independently 1, 2, or 3.

In certain embodiments, the compound of Formula (H I-c) is of Formula (H I-c-2):

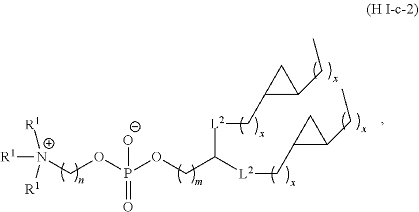

or a salt thereof.

In certain embodiments, the compound of Formula (I-c) is of the following formula:

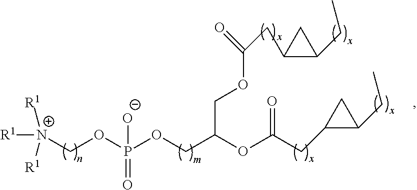

or a salt thereof.

In certain embodiments, the compound of Formula (H I-c) is the following:

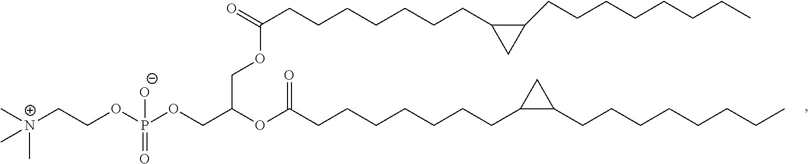 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H I-c) is of Formula (H I-c-3):

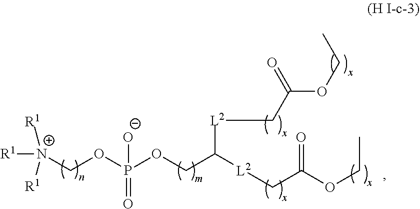

or a salt thereof.

In certain embodiments, the compound of Formula (H I-c) is of the following formulae:

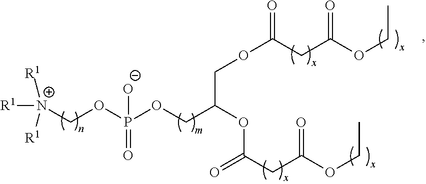

or a salt thereof.

In certain embodiments, the compound of Formula (H I-c) is the following:

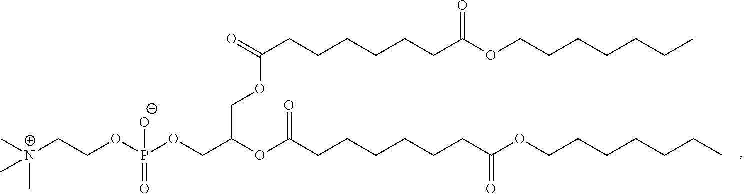 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

### Phosphocholine Linker Modifications

In certain embodiments, a phospholipid useful in the present invention comprises a modified phosphocholine moiety, wherein the alkyl chain linking the quaternary amine to the phosphoryl group is not ethylene (e.g., n is not 2). Therefore, in certain embodiments, a phospholipid useful in the present invention is a compound of Formula (H I), wherein n is 1, 3, 4, 5, 6, 7, 8, 9, or 10. For example, in certain embodiments, a compound of Formula (H I) is of one of the following formulae:

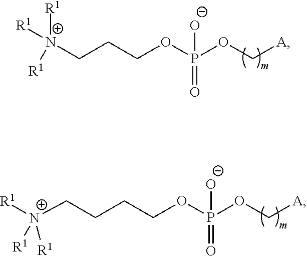

or a salt thereof.

In certain embodiments, a compound of Formula (H I) is one of the following:

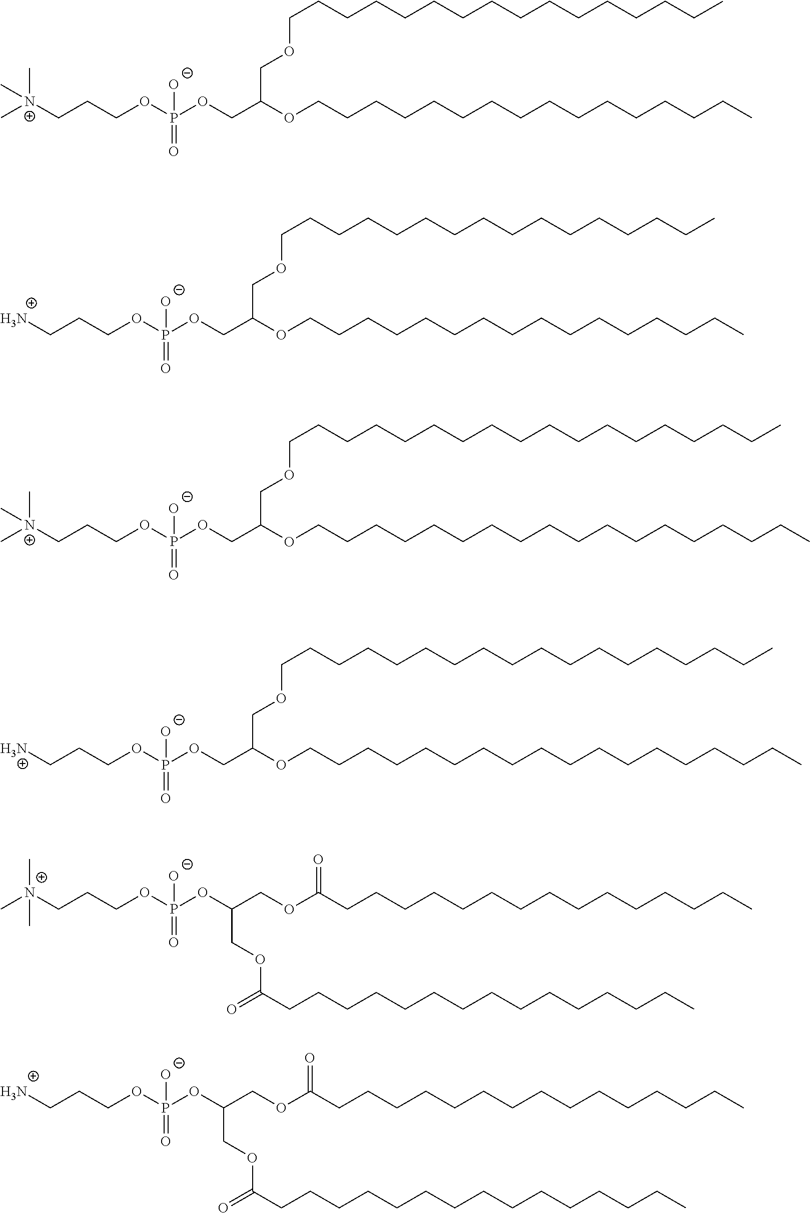

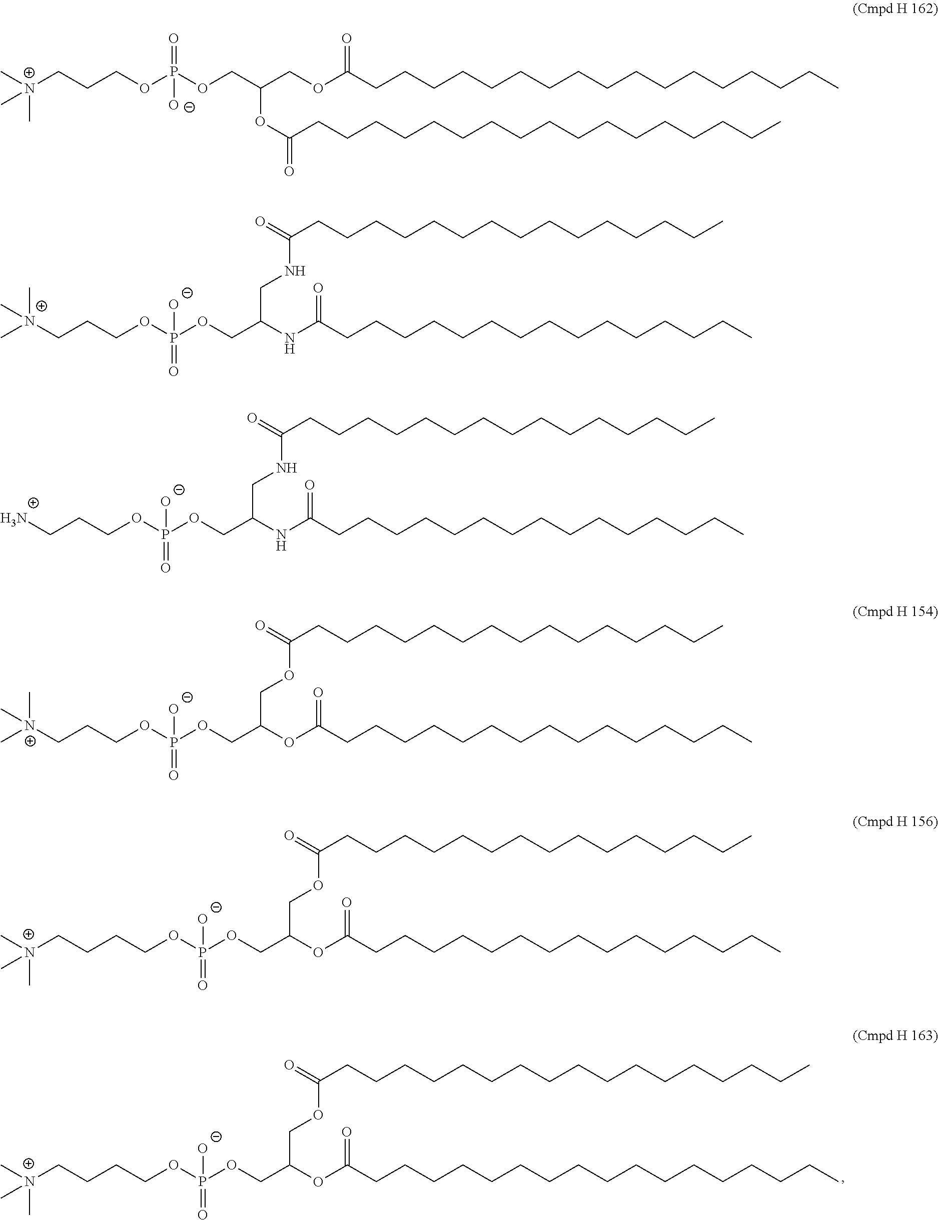

or salts thereof.

Numerous LNP formulations having phospholipids other than DSPC were prepared and tested for activity, as demonstrated in the examples below.

### Phospholipid Substitute or Replacement

In some embodiments, the lipid-based composition (e.g., lipid nanoparticle) comprises an oleic acid or an oleic acid analog in place of a phospholipid. In some embodiments, an oleic acid analog comprises a modified oleic acid tail, a modified carboxylic acid moiety, or both. In some embodiments, an oleic acid analog is a compound wherein the carboxylic acid moiety of oleic acid is replaced by a different group.

In some embodiments, the lipid-based composition (e.g., lipid nanoparticle) comprises a different zwitterionic group in place of a phospholipid.

Exemplary phospholipid substitutes and/or replacements are provided in Published PCT Application WO 2017/099823, herein incorporated by reference.

Exemplary phospholipid substitutes and/or replacements are provided in Published PCT Application WO 2017/099823, herein incorporated by reference.

(iv) PEG Lipids

Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DS G), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG2k-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A₂, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

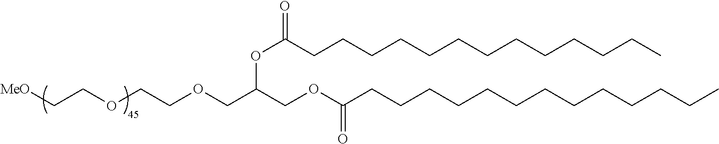

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In some embodiments, the PEG lipid is a compound of Formula (PI):

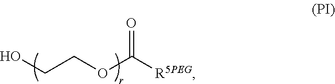

or a salt or isomer thereof, wherein:

r is an integer between 1 and 100; R^{5PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group.

For example, R^{5PEG} is C₁₇ alkyl. For example, the PEG lipid is a compound of Formula (PI-a):

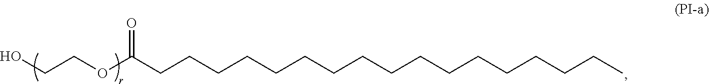

or a salt or isomer thereof, wherein r is an integer between 1 and 100.

For example, the PEG lipid is a compound of the following formula:

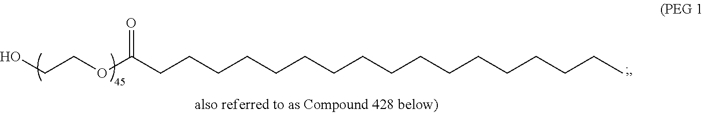

or a salt or isomer thereof.

The PEG lipid may be a compound of Formula (PII):

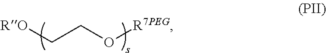

or a salt or isomer thereof, wherein:

s is an integer between 1 and 100;

R″ is a hydrogen, C₁₋₁₀ alkyl, or an oxygen protecting group; R^{7PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁-6 alkyl, or a nitrogen protecting group.

In some embodiments, R^{7PEG} is C₁₀₋₆₀ alkyl, and one or more of the methylene groups of R^{7PEG} are replaced with —C(O)—. For example, R^{7PEG} is C₃₁ alkyl, and two of the methylene groups of R^{7PEG} are replaced with —C(O)—.

In some embodiments, R″ is methyl.

In some embodiments, the PEG lipid is a compound of Formula (PII-a):

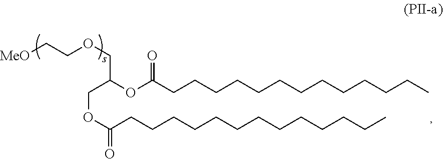

or a salt or isomer thereof, wherein s is an integer between 1 and 100.

For example, the PEG lipid is a compound of the following formula:

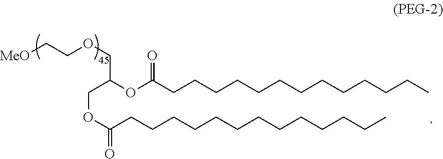

or a salt or isomer thereof.

In certain embodiments, a PEG lipid useful in the present invention is a compound of Formula (PIII). Provided herein are compounds of Formula (PIII):

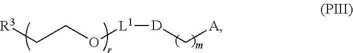

or salts thereof, wherein:

R³ is —OR^{O};

R^{O} is hydrogen, optionally substituted alkyl, or an oxygen protecting group;

r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, 0, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

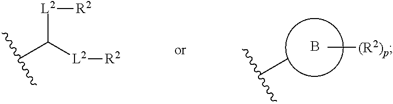

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), 0, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂₀, OS(O)₂₀, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (PIII) is a PEG-OH lipid (i.e., R³ is —OR^{O}, and R^{O} is hydrogen). In certain embodiments, the compound of Formula (PIII) is of Formula (PIII-OH):

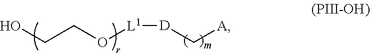

or a salt thereof.

In certain embodiments, D is a moiety obtained by click chemistry (e.g., triazole). In certain embodiments, the compound of Formula (PIII) is of Formula (PIII-a-1) or (PIII-a-2):

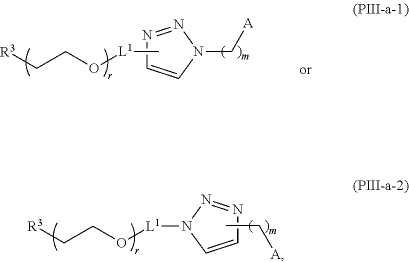

or a salt thereof.

In certain embodiments, the compound of Formula (PIII) is of one of the following formulae:

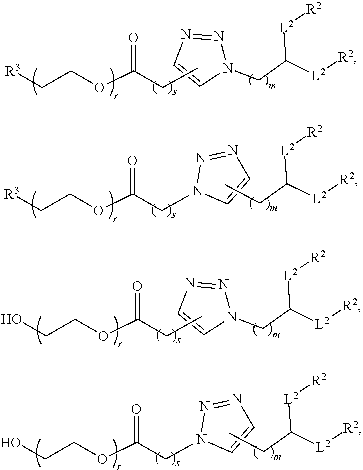

or a salt thereof, wherein

s is 0, 1, 2, 3, 4, 5, 6, 7, 8, 9, or 10.

In certain embodiments, the compound of Formula (PIII) is of one of the following formulae:

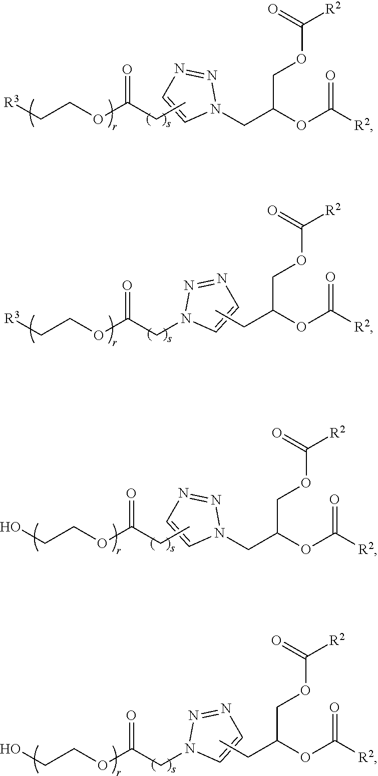

or a salt thereof.

In certain embodiments, a compound of Formula (PIII) is of one of the following formulae:

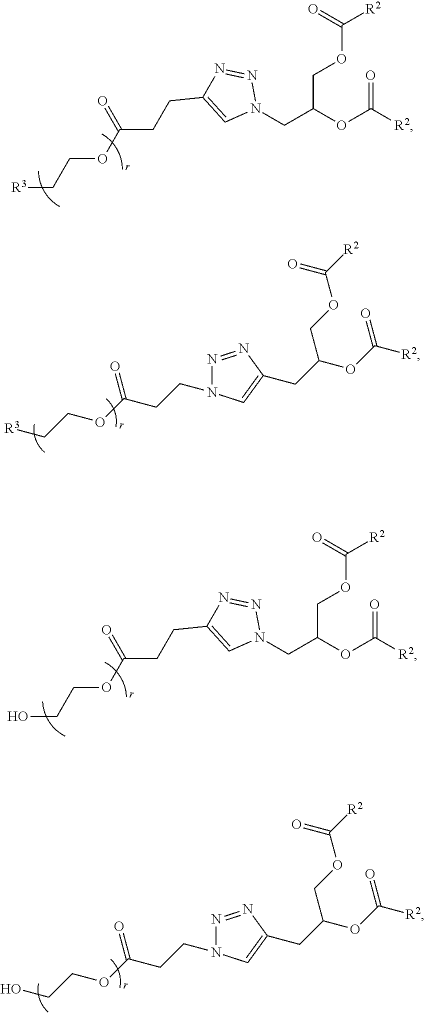

or a salt thereof.

In certain embodiments, a compound of Formula (PIII) is of one of the following formulae:

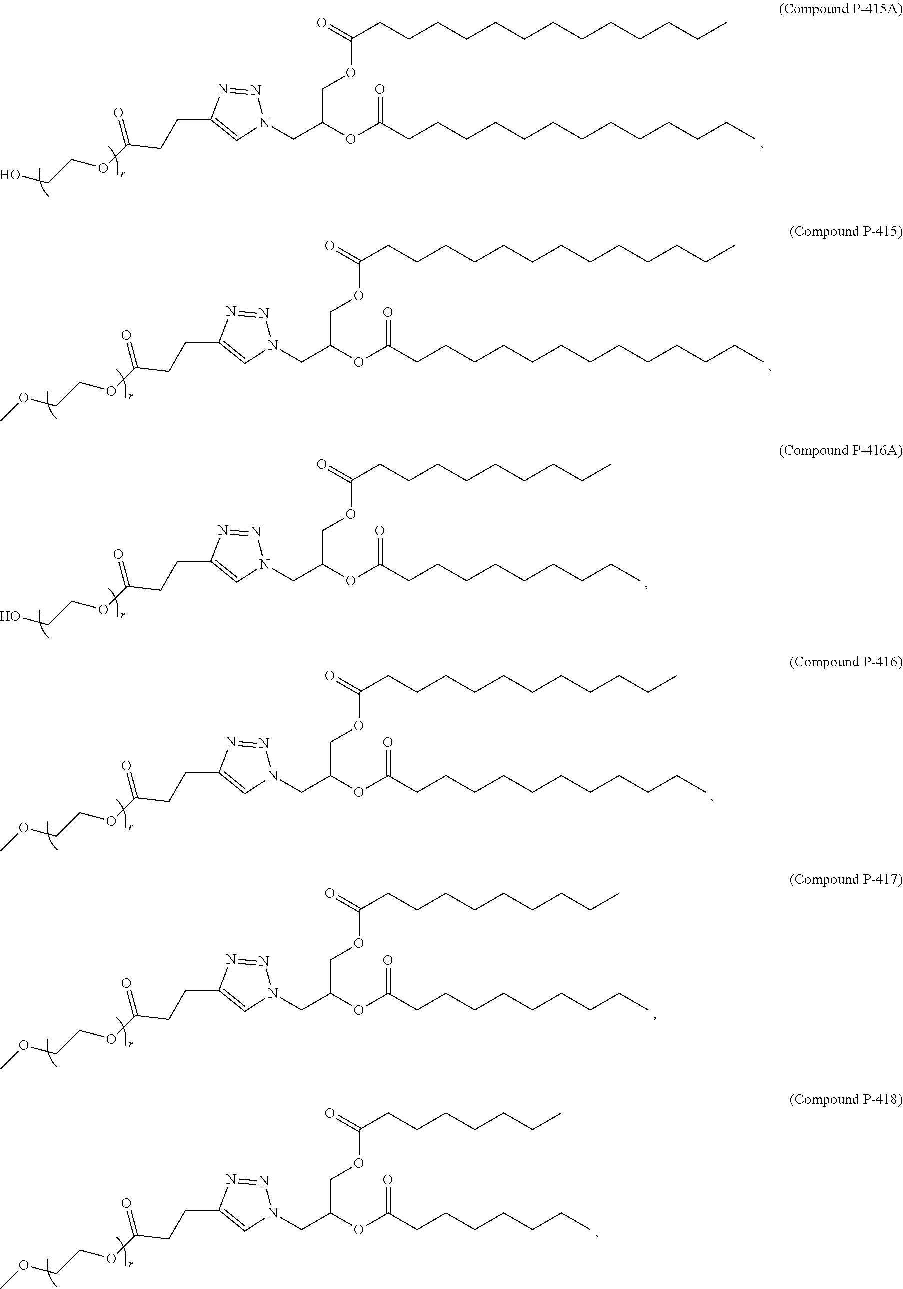

or a salt thereof.

In certain embodiments, D is a moiety cleavable under physiological conditions (e.g., ester, amide, carbonate, carbamate, urea). In certain embodiments, a compound of Formula (PIII) is of Formula (PIII-b-1) or (PIII-b-2):

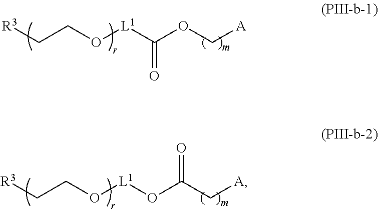

or a salt thereof.

In certain embodiments, a compound of Formula (PIII) is of Formula (PIII-b-1-OH) or (PIII-b-2-0H):

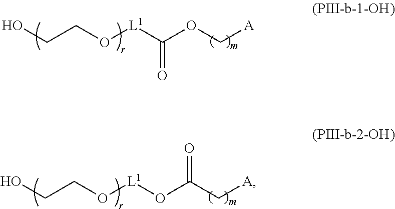

or a salt thereof.

In certain embodiments, the compound of Formula (PIII) is of one of the following formulae:

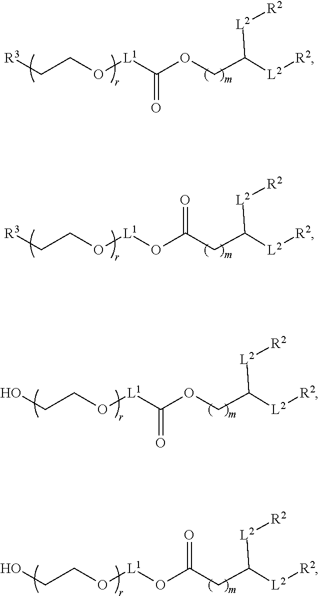

or a salt thereof.

In certain embodiments, a compound of Formula (PIII) is of one of the following formulae:

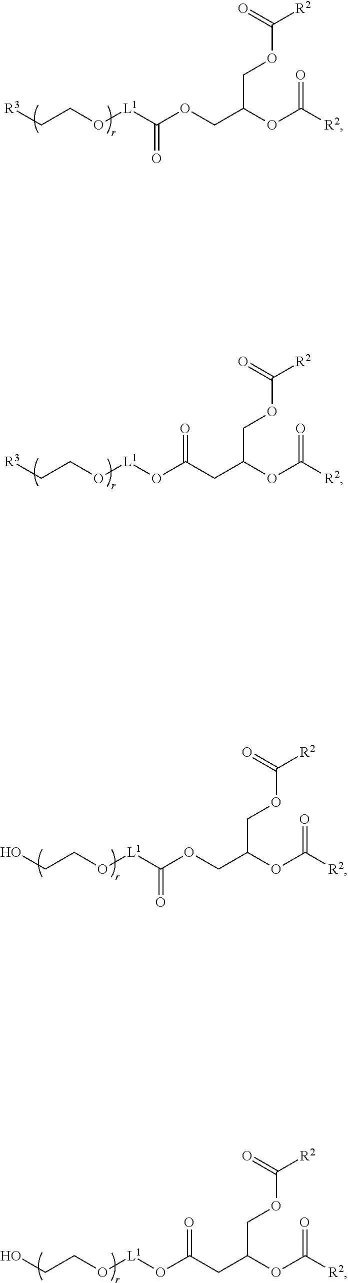

or a salt thereof.

In certain embodiments, a compound of Formula (PIII) is of one of the following formulae:

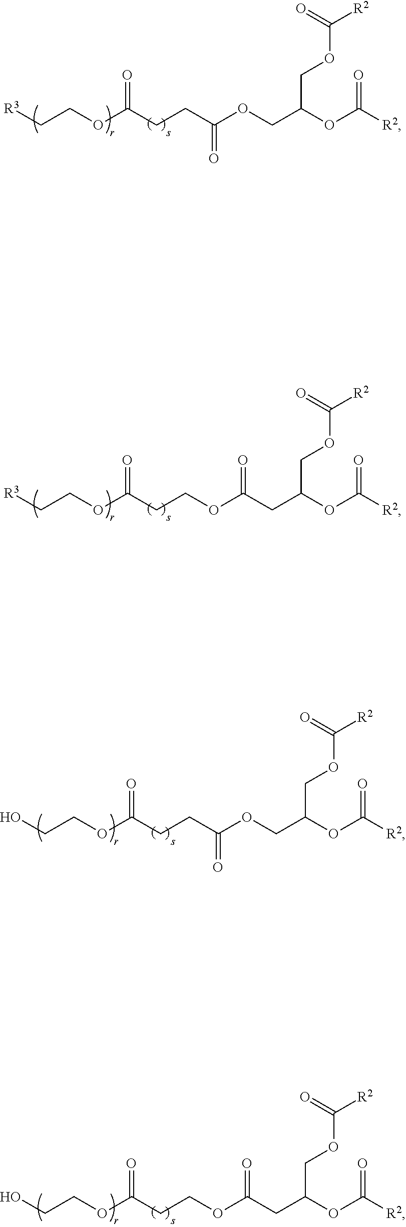

or a salt thereof.

In certain embodiments, a compound of Formula (PIII) is of one of the following formulae:

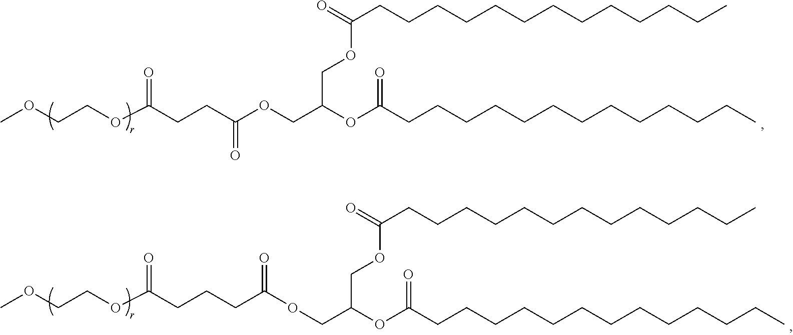

or salts thereof.

In certain embodiments, a PEG lipid useful in the present invention is a PEGylated fatty acid. In certain embodiments, a PEG lipid useful in the present invention is a compound of Formula (PIV). Provided herein are compounds of Formula (PIV):

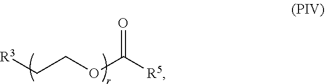

or a salts thereof, wherein:

R³ is —OR^{O};

R^{O} is hydrogen, optionally substituted alkyl or an oxygen protecting group;

r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and

each instance of R^{N} is independently hydrogen, optionally substituted alkyl, or a nitrogen protecting group.

In certain embodiments, the compound of Formula (PIV is of Formula (PIV-OH):

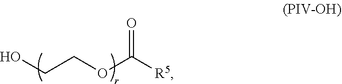

or a salt thereof. In some embodiments, r is 40-50. In some embodiments, r is 45.

In certain embodiments, a compound of Formula (PIV) is of one of the following formulae:

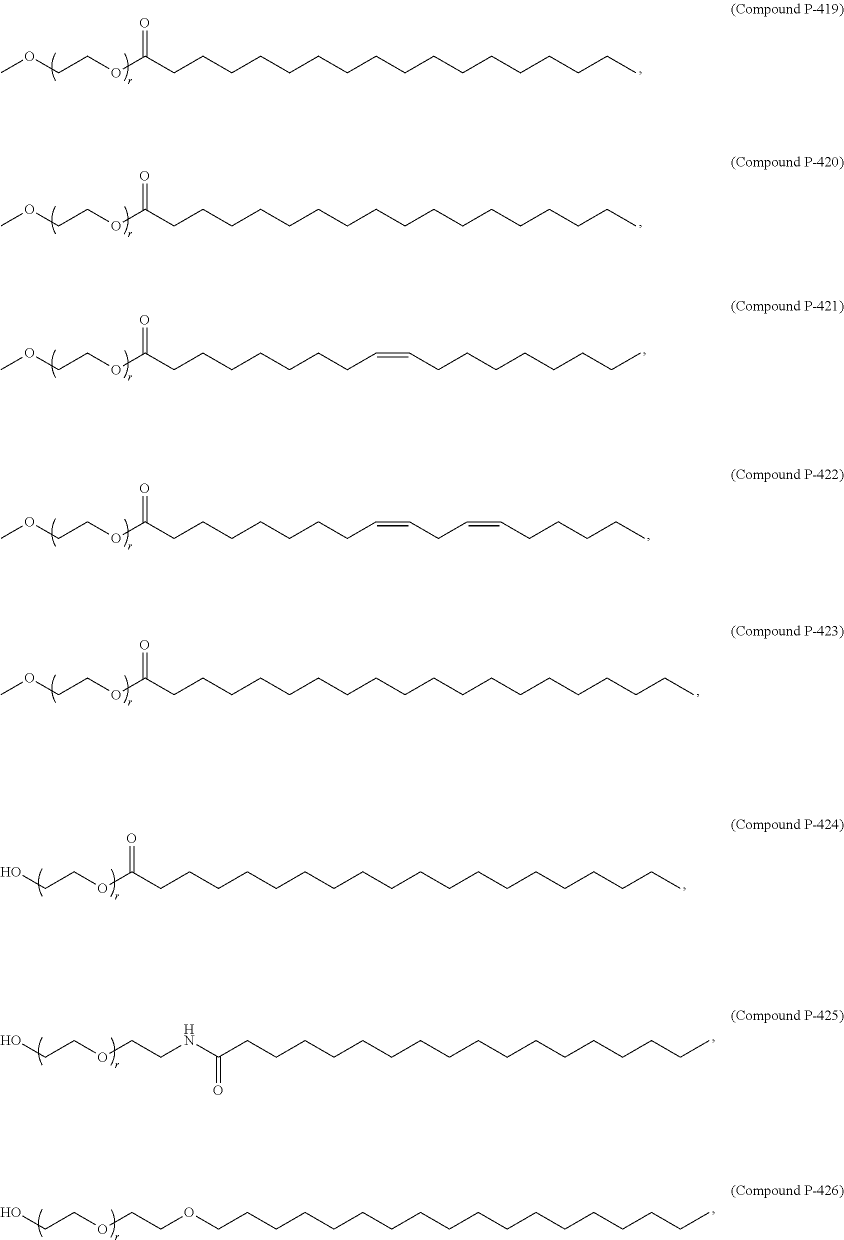

or a salt thereof. In some embodiments, r is 40-50. In some embodiments, r is 45.

In yet other embodiments the compound of Formula (PIV) is:

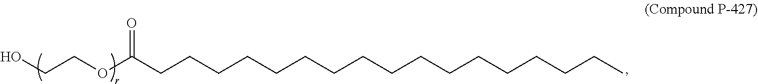

or a salt thereof.

In one embodiment, the compound of Formula (PIV) is

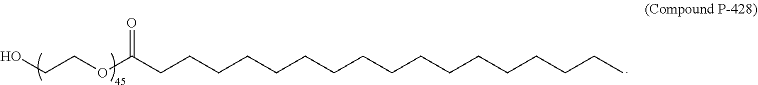

In one aspect, provided herein are lipid nanoparticles (LNPs) comprising PEG lipids of Formula (PV):

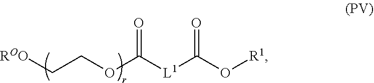

or pharmaceutically acceptable salts thereof; wherein:
L¹ is a bond, optionally substituted C₁₋₃ alkylene, optionally substituted C₁-3 heteroalkylene, optionally substituted C₂₋₃ alkenylene, optionally substituted C₂₋₃ alkynylene;
R¹ is optionally substituted C₅₋₃₀ alkyl, optionally substituted C₅₋₃₀ alkenyl, or optionally substituted C₅₋₃₀ alkynyl;
R^{O} is hydrogen, optionally substituted alkyl, optionally substituted acyl, or an oxygen protecting group; and
r is an integer from 2 to 100, inclusive.

In certain embodiments, the PEG lipid of Formula (PV) is of the following formula:

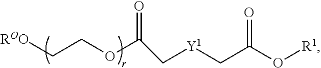

or a pharmaceutically acceptable salt thereof; wherein:
Y¹ is a bond, —CR₂—, —O—, —NR^{N}—, or —S—;

each instance of R is independently hydrogen, halogen, or optionally substituted alkyl;

and

R^{N} is hydrogen, optionally substituted alkyl, optionally substituted acyl, or a nitrogen protecting group.

In certain embodiments, the PEG lipid of Formula (PV) is of one of the following formulae:

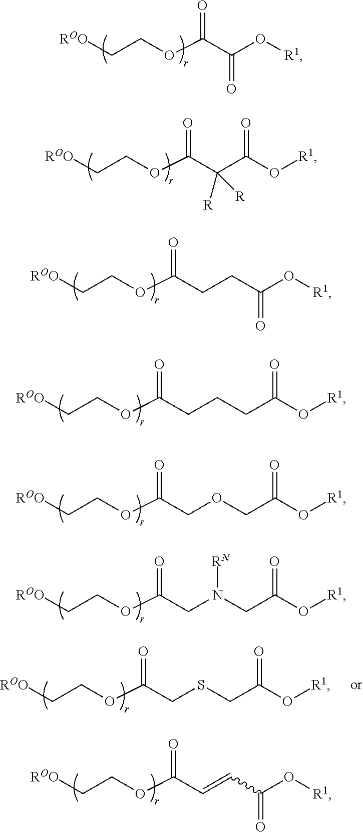

or a pharmaceutically acceptable salt thereof, wherein:

each instance of R is independently hydrogen, halogen, or optionally substituted alkyl.

In certain embodiments, the PEG lipid of Formula (PV) is of one of the following formulae:

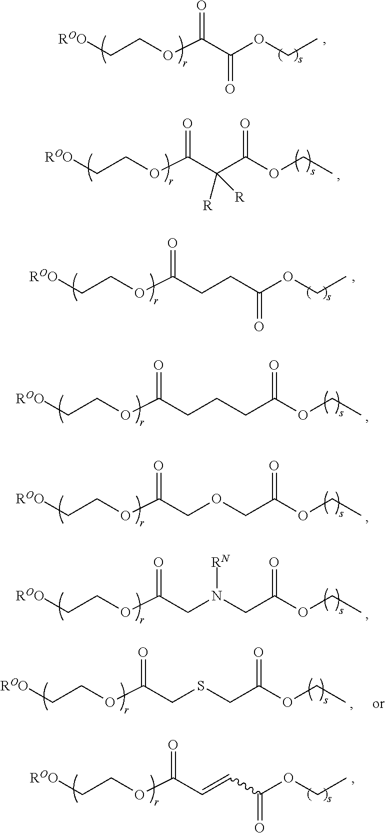

or a pharmaceutically acceptable salt thereof; wherein:
s is an integer from 5-25, inclusive.

In certain embodiments, the PEG lipid of Formula (PV) is of one of the following formulae:

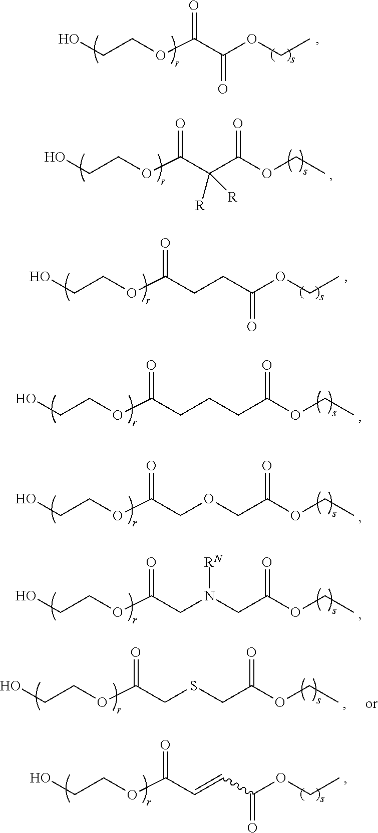

or a pharmaceutically acceptable salt thereof.

In certain embodiments, the PEG lipid of Formula (PV) is selected from the group consisting of:

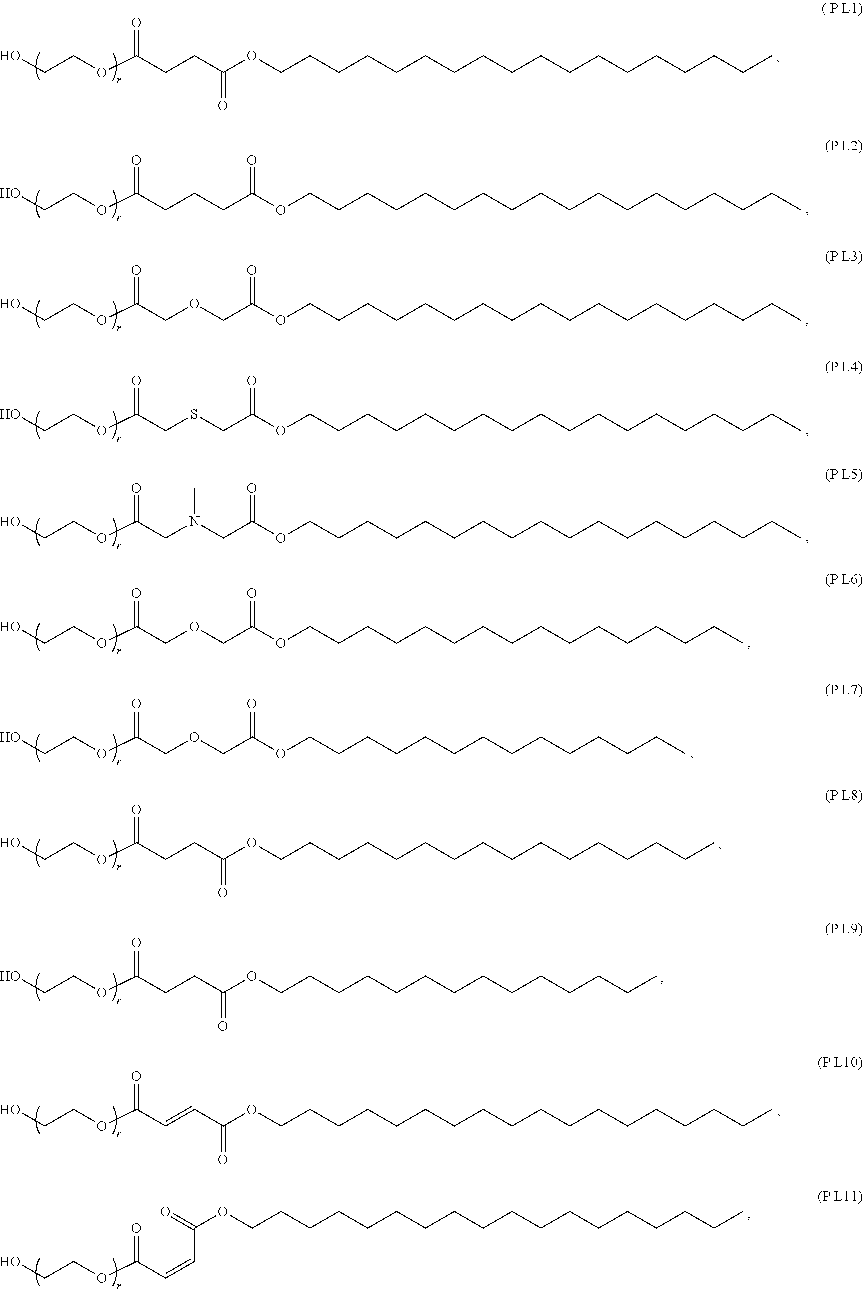

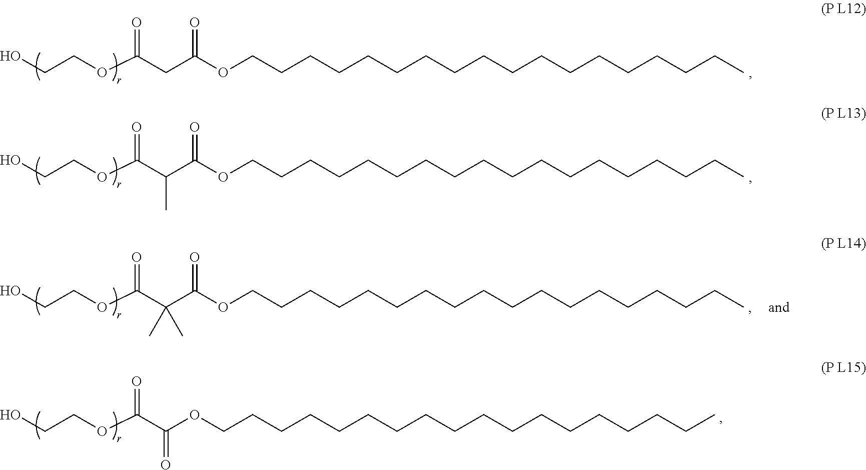

and pharmaceutically acceptable salts thereof.

In another aspect, provided herein are lipid nanoparticles (LNPs) comprising PEG lipids of Formula (PVI):

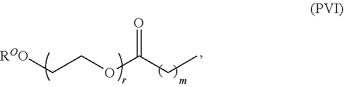

or pharmaceutically acceptable salts thereof; wherein:
R^{O} is hydrogen, optionally substituted alkyl, optionally substituted acyl, or an oxygen protecting group;
r is an integer from 2 to 100, inclusive; and
m is an integer from 5-15, inclusive, or an integer from 19-30, inclusive.

In certain embodiments, the PEG lipid of Formula (PVI) is of one of the following formulae:

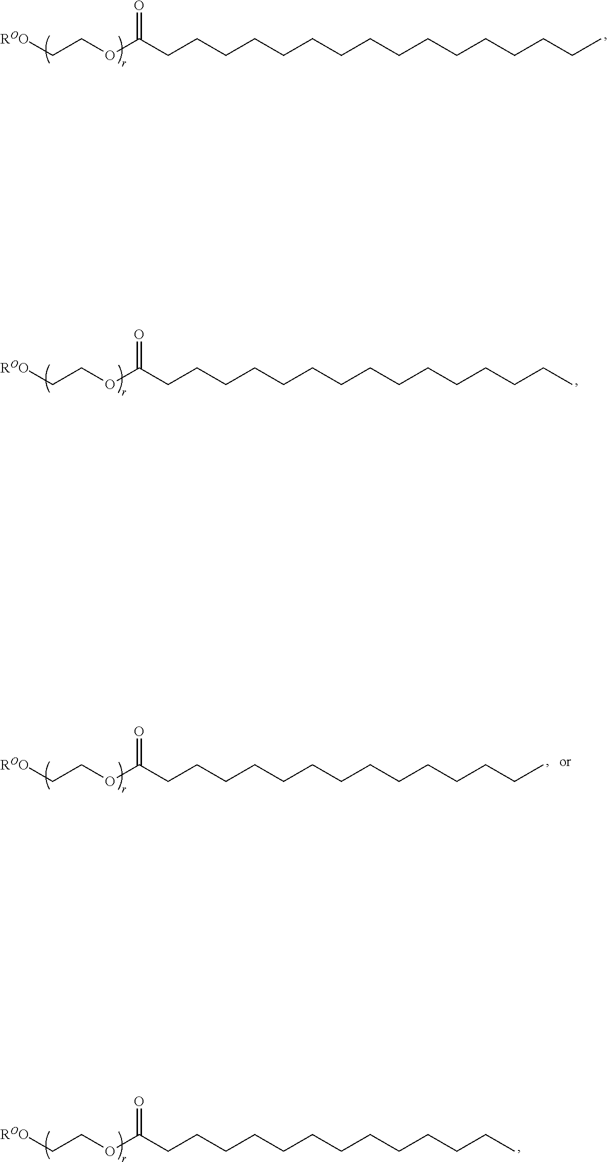

or a pharmaceutically acceptable salt thereof.

In certain embodiments, the PEG lipid of Formula (PVI) is of one of the following formulae:

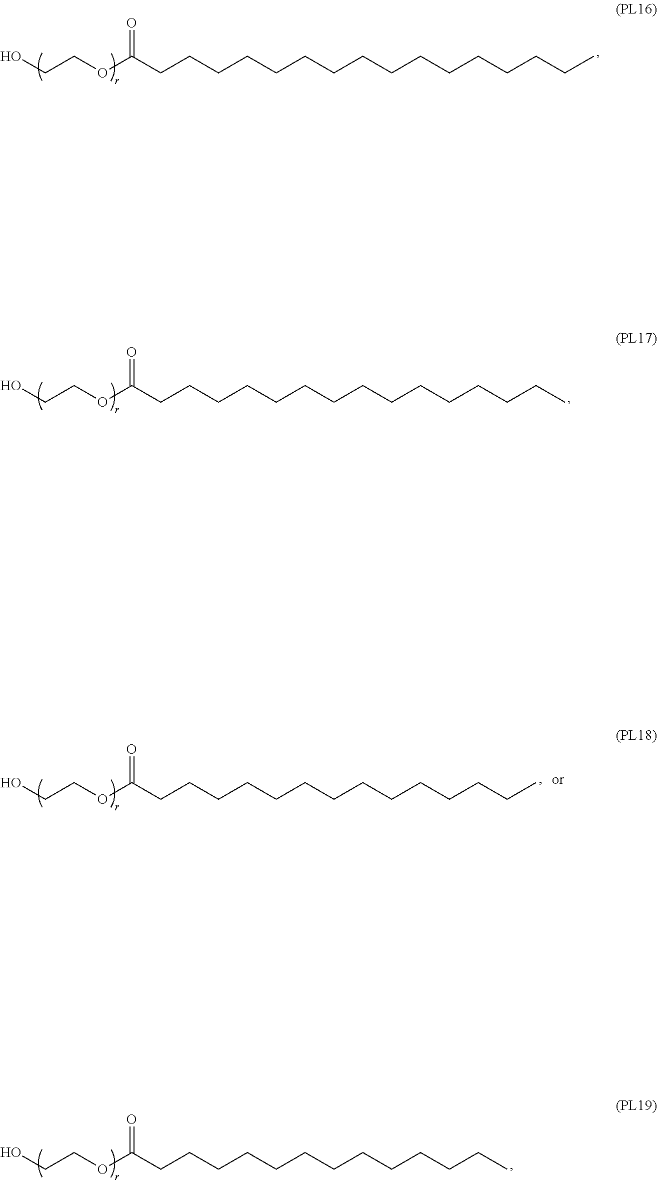

or a pharmaceutically acceptable salt thereof.

In another aspect, provided herein are lipid nanoparticles (LNPs) comprising PEG lipids of Formula (PVII):

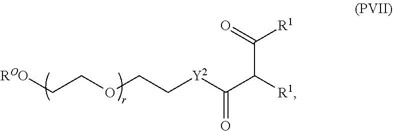

or pharmaceutically acceptable salts thereof, wherein:
Y² is —O—, —NR^{N}—, or —S—
each instance of R¹ is independently optionally substituted C₅₋₃₀ alkyl, optionally substituted C₅₋₃₀ alkenyl, or optionally substituted C₅₋₃₀ alkynyl;
R^{O} is hydrogen, optionally substituted alkyl, optionally substituted acyl, or an oxygen protecting group;
R^{N} is hydrogen, optionally substituted alkyl, optionally substituted acyl, or a nitrogen protecting group; and
r is an integer from 2 to 100, inclusive.

In certain embodiments, the PEG lipid of Formula (PVII) is of one of the following formulae:

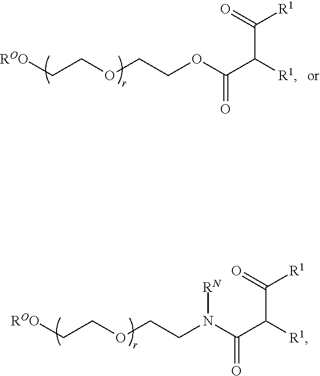

or a pharmaceutically acceptable salt thereof.

In certain embodiments, the PEG lipid of Formula (PVII) is of one of the following formulae:

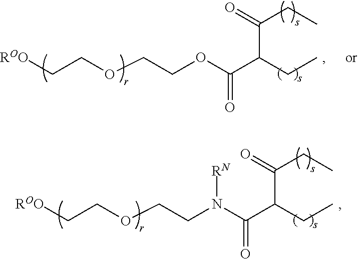

or a pharmaceutically acceptable salt thereof; wherein:
each instance of s is independently an integer from 5-25, inclusive.

In certain embodiments, the PEG lipid of Formula (PVII) is of one of the following formulae:

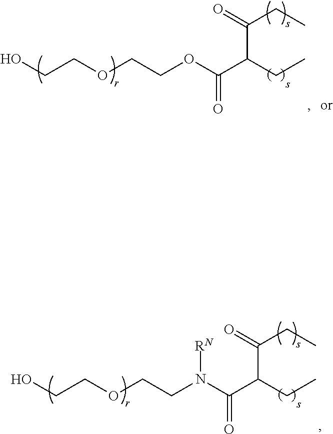

or a pharmaceutically acceptable salt thereof.

In certain embodiments, the PEG lipid of Formula (PVII) is selected from the group consisting of:

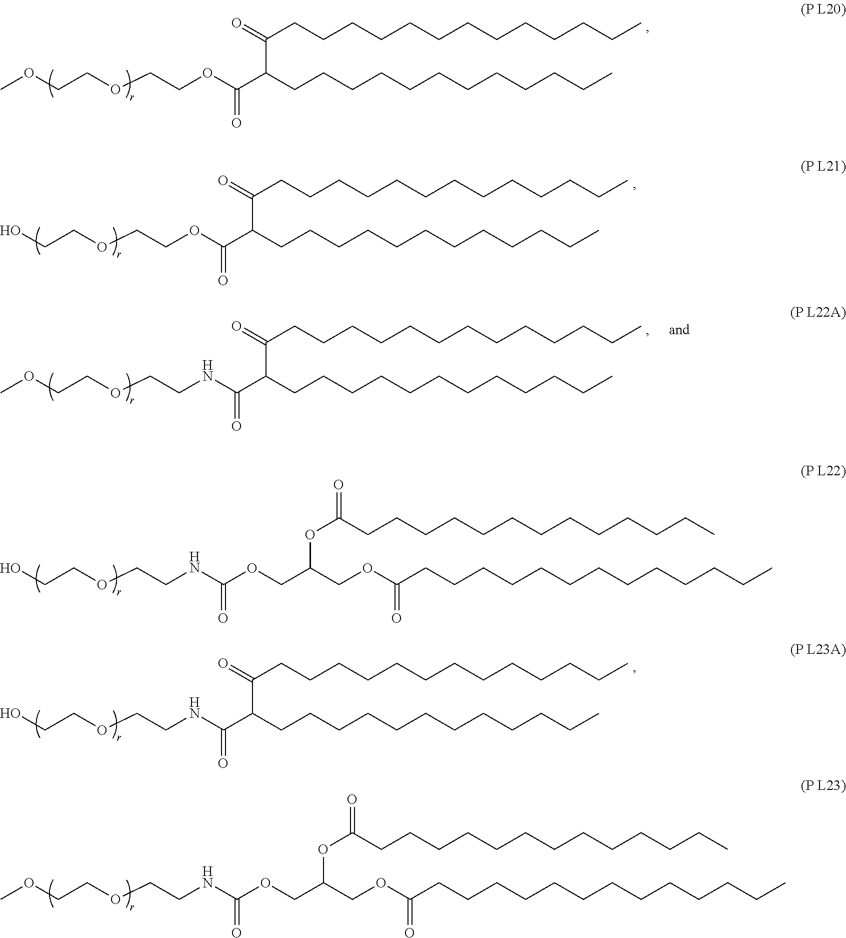

and pharmaceutically acceptable salts thereof.

In another aspect, provided herein are lipid nanoparticles (LNPs) comprising PEG lipids of Formula (PVIII):

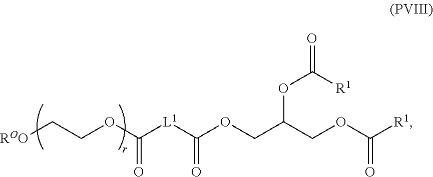

or pharmaceutically acceptable salts thereof, wherein:
L¹ is a bond, optionally substituted C₁₋₃ alkylene, optionally substituted C₁₋₃ heteroalkylene, optionally substituted C₂₋₃ alkenylene, optionally substituted C₂₋₃ alkynylene;
each instance of R¹ is independently optionally substituted C₅₋₃₀ alkyl, optionally substituted C₃₋₃₀ alkenyl, or optionally substituted C₅₋₃₀ alkynyl;
R^{O} is hydrogen, optionally substituted alkyl, optionally substituted acyl, or an oxygen protecting group;
r is an integer from 2 to 100, inclusive;
provided that when L¹ is —CH₂CH₂— or —CH₂CH₂CH₂—, R^{O} is not methyl.

In certain embodiments, when L¹ is optionally substituted C₂ or C₃ alkylene, R^{O} is not optionally substituted alkyl. In certain embodiments, when L¹ is optionally substituted C₂ or C₃ alkylene, R^{O} is hydrogen. In certain embodiments, when L¹ is —CH₂CH₂— or —CH₂CH₂CH₂—, R^{O} is not optionally substituted alkyl. In certain embodiments, when L¹ is —CH₂CH₂— or —CH₂CH₂CH₂—, R^{O} is hydrogen.

In certain embodiments, the PEG lipid of Formula (PVIII) is of the formula:

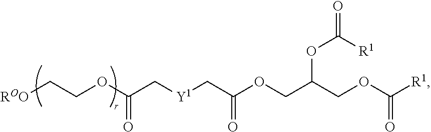

or a pharmaceutically acceptable salt thereof, wherein:

Y¹ is a bond, —CR₂—, —O—, —NR^{N}—, or —S—;

each instance of R is independently hydrogen, halogen, or optionally substituted alkyl;

R^{N} is hydrogen, optionally substituted alkyl, optionally substituted acyl, or a nitrogen protecting group;

provided that when Y¹ is a bond or —CH₂—, R^{O} is not methyl.

In certain embodiments, when L¹ is —CR₂—, R^{O} is not optionally substituted alkyl. In certain embodiments, when L¹ is —CR₂—, R^{O} is hydrogen. In certain embodiments, when L¹ is —CH₂—, R^{O} is not optionally substituted alkyl. In certain embodiments, when L¹ is —CH₂—, R^{O} is hydrogen.

In certain embodiments, the PEG lipid of Formula (PVIII) is of one of the following formulae:

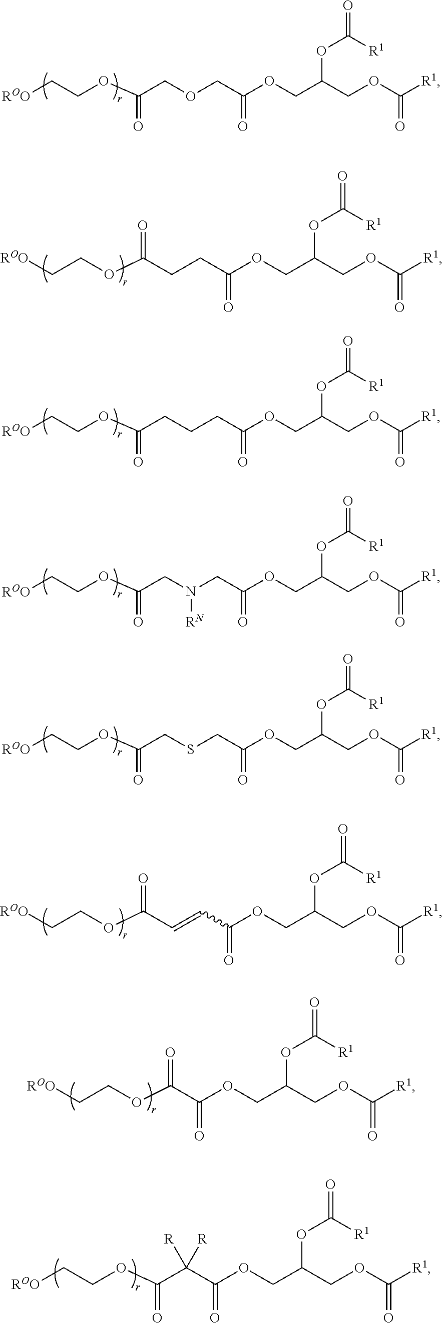

or a pharmaceutically acceptable salt thereof, wherein:
each instance of R is independently hydrogen, halogen, or optionally substituted alkyl.

In certain embodiments, the PEG lipid of Formula (PVIII) is of one of the following formulae:

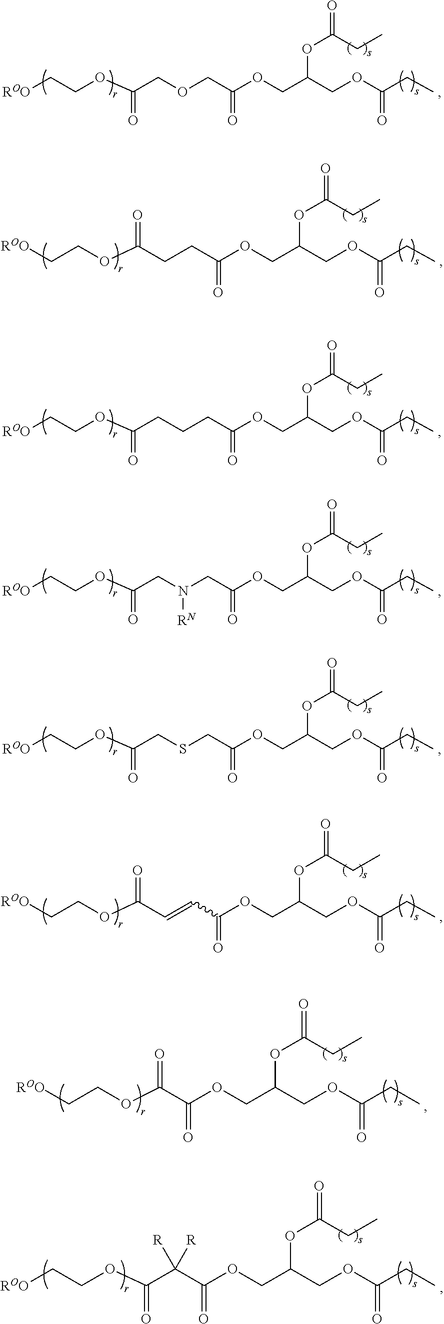

or a pharmaceutically acceptable salt thereof; wherein:
each instance of R is independently hydrogen, halogen, or optionally substituted alkyl; and
each s is independently an integer from 5-25, inclusive.

In certain embodiments, the PEG lipid of Formula (PVIII) is of one of the following formulae:

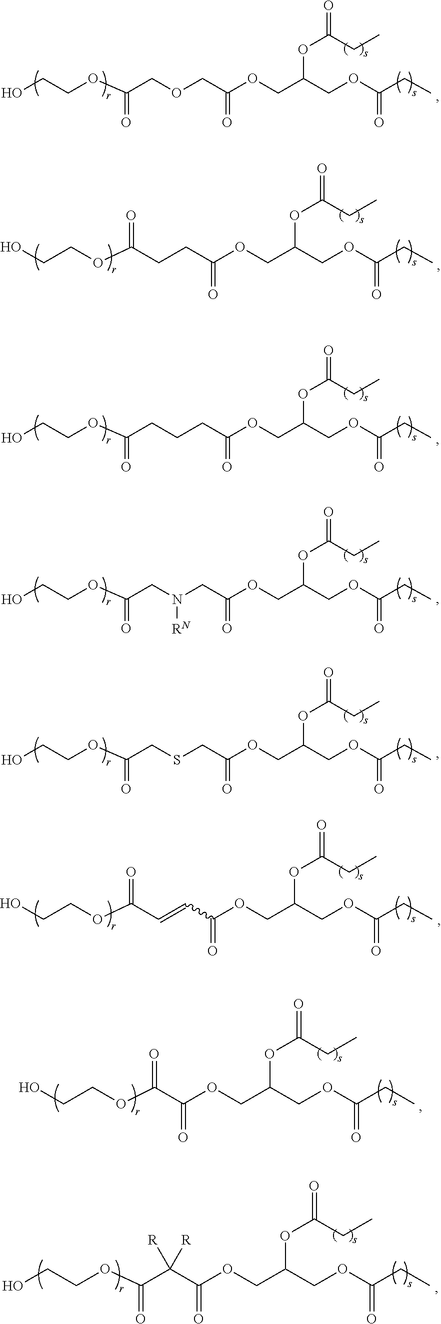

or a pharmaceutically acceptable salt thereof.

In certain embodiments, the PEG lipid of Formula (PVIII) is selected from the group consisting of:

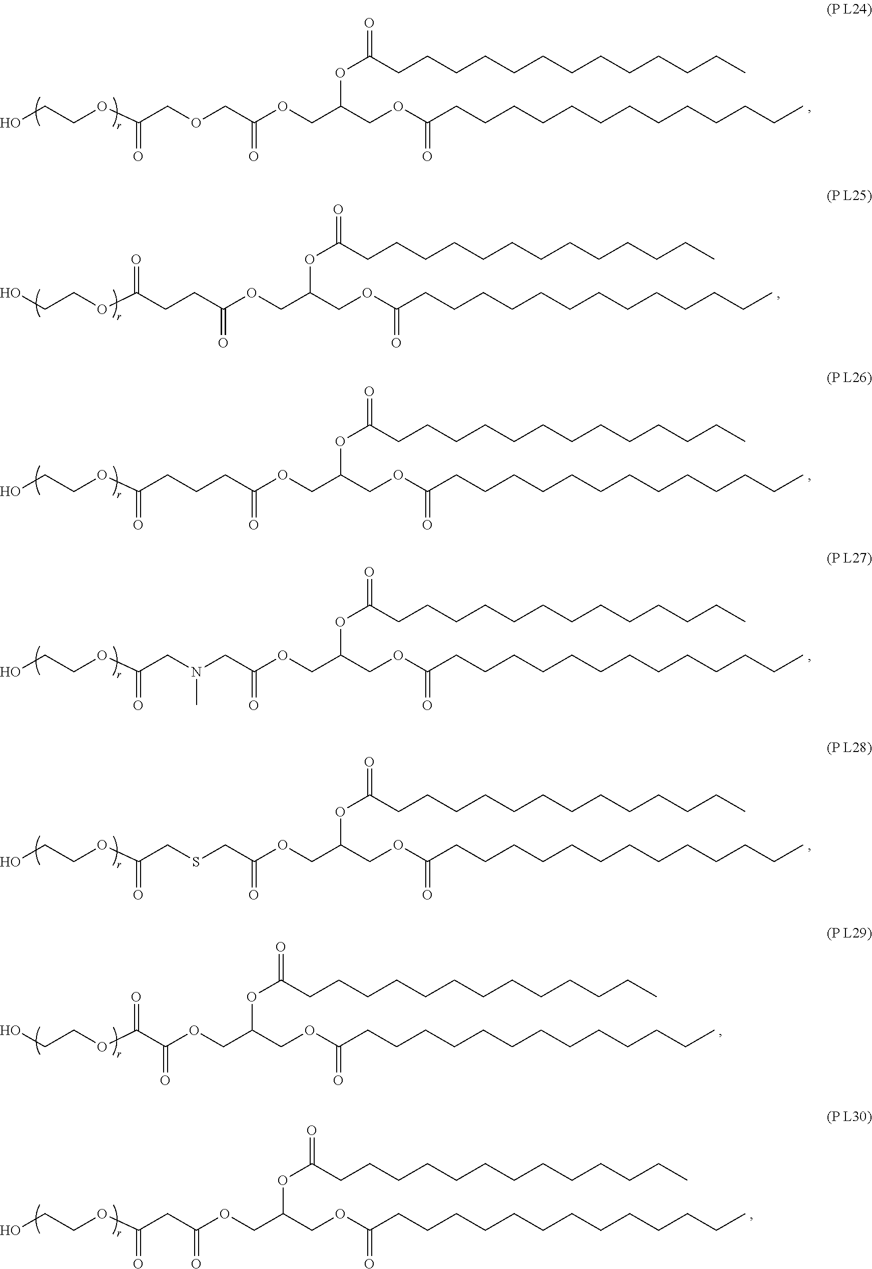

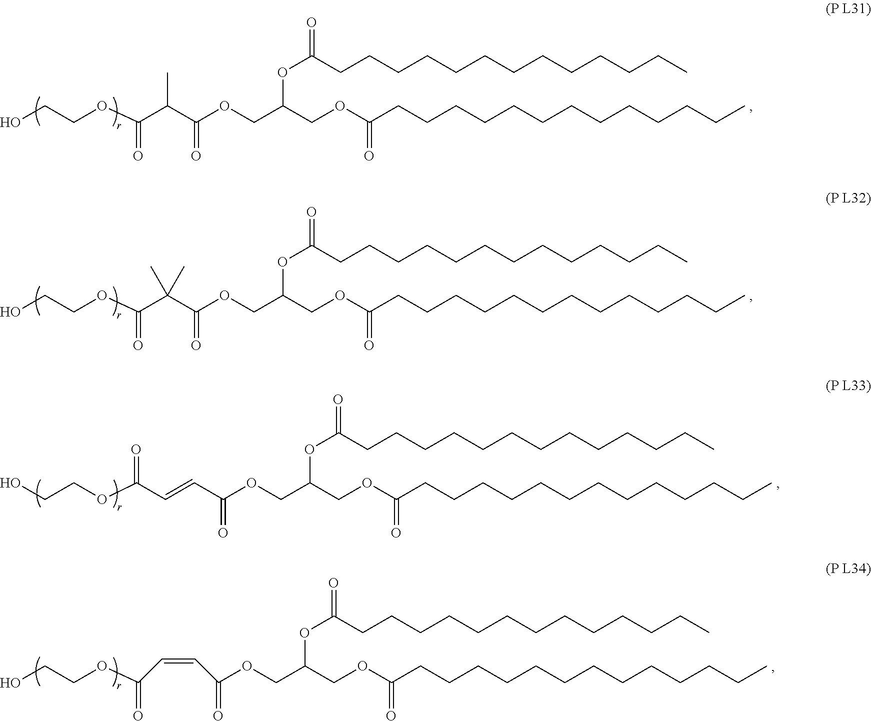

and pharmaceutically acceptable salts thereof.

In any of the foregoing or related aspects, a PEG lipid of the invention is featured wherein r is 40-50.

The LNPs provided herein, in certain embodiments, exhibit increased PEG shedding compared to existing LNP formulations comprising PEG lipids. “PEG shedding,” as used herein, refers to the cleavage of a PEG group from a PEG lipid. In many instances, cleavage of a PEG group from a PEG lipid occurs through serum-driven esterase-cleavage or hydrolysis. The PEG lipids provided herein, in certain embodiments, have been designed to control the rate of PEG shedding. In certain embodiments, an LNP provided herein exhibits greater than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits greater than 50% PEG shedding after about 6 hours in human serum. In certain embodiments, an LNP provided herein exhibits greater than 60% PEG shedding after about 6 hours in human serum. In certain embodiments, an LNP provided herein exhibits greater than 70% PEG shedding after about 6 hours in human serum. In certain embodiments, the LNP exhibits greater than 80% PEG shedding after about 6 hours in human serum. In certain embodiments, the LNP exhibits greater than 90% PEG shedding after about 6 hours in human serum. In certain embodiments, an LNP provided herein exhibits greater than 90% PEG shedding after about 6 hours in human serum.

In other embodiments, an LNP provided herein exhibits less than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits less than 60% PEG shedding after about 6 hours in human serum. In certain embodiments, an LNP provided herein exhibits less than 70% PEG shedding after about 6 hours in human serum. In certain embodiments, an LNP provided herein exhibits less than 80% PEG shedding after about 6 hours in human serum.

In addition to the PEG lipids provided herein, the LNP may comprise one or more additional lipid components. In certain embodiments, the PEG lipids are present in the LNP in a molar ratio of 0.15-15% with respect to other lipids. In certain embodiments, the PEG lipids are present in a molar ratio of 0.15-5% with respect to other lipids. In certain embodiments, the PEG lipids are present in a molar ratio of 1-5% with respect to other lipids. In certain embodiments, the PEG lipids are present in a molar ratio of 0.15-2% with respect to other lipids. In certain embodiments, the PEG lipids are present in a molar ratio of 1-2% with respect to other lipids. In certain embodiments, the PEG lipids are present in a molar ratio of approximately 1%, 1.1%, 1.2%, 1.3%, 1.4%, 1.5%, 1.6%, 1.7%, 1.8%, 1.9%, or 2% with respect to other lipids. In certain embodiments, the PEG lipids are present in a molar ratio of approximately 1.5% with respect to other lipids.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 2 mol %. In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

### Exemplary Synthesis

### Compound: HO-PEG2000-ester-C18

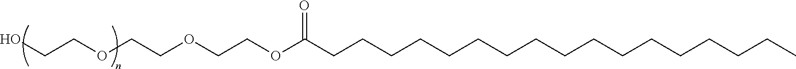

To a nitrogen filled flask containing palladium on carbon (10 wt. %, 74 mg, 0.070 mmol) was added Benzyl-PEG2000-ester-C18 (822 mg, 0.35 mmol) and MeOH (20 mL). The flask was evacuated and backfilled with H₂ three times, and allowed to stir at RT and 1 atm H2 for 12 hours. The mixture was filtered through celite, rinsing with DCM, and the filtrate was concentrated in vacuo to provide the desired product (692 mg, 88%). Using this methodology n=40-50. In one embodiment, n of the resulting polydispersed mixture is referred to by the average, 45.

For example, the value of r can be determined on the basis of a molecular weight of the PEG moiety within the PEG lipid. For example, a molecular weight of 2,000 (e.g., PEG2000) corresponds to a value of n of approximately 45. For a given composition, the value for n can connote a distribution of values within an art-accepted range, since polymers are often found as a distribution of different polymer chain lengths. For example, a skilled artisan understanding the polydispersity of such polymeric compositions would appreciate that an n value of 45 (e.g., in a structural formula) can represent a distribution of values between 40-50 in an actual PEG-containing composition, e.g., a DMG PEG200 peg lipid composition.

In some aspects, an immune cell delivery lipid of the pharmaceutical compositions disclosed herein does not comprise a PEG-lipid.

In one embodiment, an immune cell delivery LNP of the disclosure comprises a PEG-lipid. In one embodiment, the PEG lipid is not PEG DMG. In some aspects, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some aspects, the PEG lipid is selected from the group consisting of PEG-c-DOMG, PEG-DMG, PEG-DLPE, PEG-DMPE, PEG-DPPC and PEG-DSPE lipid. In other aspects, the PEG-lipid is PEG-DMG.

In one embodiment, an immune cell delivery LNP of the disclosure comprises a PEG-lipid which has a chain length longer than about 14 or than about 10, if branched.

In one embodiment, the PEG lipid is a compound selected from the group consisting of any of Compound Nos. P415, P416, P417, P 419, P 420, P 423, P 424, P 428, P L1, P L2, P L16, P L17, P L18, P L19, P L22 and P L23. In one embodiment, the PEG lipid is a compound selected from the group consisting of any of Compound Nos. P415, P417, P 420, P 423, P 424, P 428, P L1, P L2, P L16, P L17, P L18, P L19, P L22 and P L23.

In one embodiment, a PEG lipid is selected from the group consisting of: Cmpd 428, PL16, PL17, PL 18, PL19, PL 1, and PL 2.

### Immune Cell Delivery Potentiating Lipids

An effective amount of the immune cell delivery potentiating lipid in an LNP enhances delivery of the agent to an immune cell (e.g., a human or primate immune cell) relative to an LNP lacking the immune cell delivery potentiating lipid, thereby creating an immune cell delivery LNP. Immune cell delivery potentiating lipids can be characterized in that, when present in an LNP, they promote delivery of the agent present in the LNP to immune cells as compared to a control LNP lacking the immune cell delivery potentiating lipid.

In one embodiment, the presence of at least one immune cell delivery potentiating lipid in an LNP results in an increase in the percentage of LNPs associated with immune cells as compared to a control LNP lacking at least one immune cell delivery potentiating lipid. In another embodiment, the presence of at least one immune cell delivery potentiating lipid in an LNP results in an increase in the delivery of a nucleic acid molecule agent to immune cells as compared to a control LNP lacking the immune cell delivery potentiating lipid. In one embodiment, the presence of at least one immune cell delivery potentiating lipid in an LNP results in an increase in the delivery of a nucleic acid molecule agent to B cells as compared to a control LNP lacking the immune cell delivery potentiating lipid. In particular, in one embodiment, the presence of at least one immune cell delivery potentiating lipid in an LNP results in an increase in the delivery of a nucleic acid molecule agent to myeloid cells as compared to a control LNP lacking the immune cell delivery potentiating lipid. In one embodiment, the presence of at least one immune cell delivery potentiating lipid in an LNP results in an increase in the delivery of a nucleic acid molecule agent to T cells as compared to a control LNP lacking the immune cell delivery potentiating lipid.

In one embodiment, the presence of at least one immune cell delivery potentiating lipid in an LNP results in an increase in the percentage of LNPs binding to C1q as compared to a control LNP lacking at least one immune cell delivery potentiating lipid. In one embodiment, the presence of at least one immune cell delivery potentiating lipid in an LNP results in an increase in the percentage of C1q-bound LNPs taken up by immune cells (e.g., opsonized by immune cells) as compared to a control LNP lacking at least one immune cell delivery potentiating lipid.

In one embodiment, when the nucleic acid molecule is an mRNA, the presence of at least one immune cell delivery potentiating lipid results in at least about 2-fold greater expression of a protein molecule encoded by the mRNA in immune cells (e.g., a T cells, B cells, monocytes) as compared to a control LNP lacking the immune cell delivery potentiating lipid.

In one embodiment, an immune cell delivery potentiating lipid is an ionizable lipid. In any of the foregoing or related aspects, the ionizable lipid (denoted by I) of the LNP of the disclosure comprises a compound included in any e.g. a compound having any of Formula (I I), (I IA), (IIB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VII-1), (I VII-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8) and/or any of Compounds X, Y, I 48, I 50, I 109, I 111, I 113, I 181, I 182, I 244, I 292, I 301, I 321, I 322, I 326, I 328, I 330, I 331, I 332 or I M.

In one embodiment, an immune cell delivery potentiating lipid is an ionizable lipid. In any of the foregoing or related aspects, the ionizable lipid of the LNP of the disclosure comprises a compound described herein as Compound X, Compound Y, Compound I-321, Compound I-292, Compound I-326, Compound I-182, Compound I-301, Compound I-48, Compound I-50, Compound I-328, Compound I-330, Compound I-109, Compound I-111 or Compound I-181.

In any of the foregoing or related aspects, the ionizable lipid of the LNP of the disclosure comprises at least one compound selected from the group consisting of: Compound Nos. I 18 (also referred to as Compound X), I 25 (also referred to as Compound Y), I 48, I 50, I 109, I 111, I 113, I 181, I 182, I 244, I 292, I 301, I 309, I 317, 1321, I 322, I 326, I 328, 1330, I 331, 1332, I 347, I 348, I 349, I 350, I 351 and I 352. In another embodiment, the ionizable lipid of the LNP of the disclosure comprises a compound selected from the group consisting of: Compound Nos. I 18 (also referred to as Compound X), I 25 (also referred to as Compound Y), I 48, I 50, I 109, I 111, I 181, I 182, I 292, I 301, I 321, I 326, I 328, and I 330. In another embodiment, the ionizable lipid of the LNP of the disclosure comprises a compound selected from the group consisting of: Compound Nos. I 182, I 301, I 321, and I 326.

It will be understood that in embodiments where the immune cell delivery potentiating lipid comprises an ionizable lipid, it may be the only ionizable lipid present in the LNP or it may be present as a blend with at least one additional ionizable lipid. That is to say that a blend of ionizable lipids (e.g., more than one that have immune cell delivery potentiating effects or one that has an immune cell delivery potentiating effect and at least one that does not) may be employed.

In one embodiment, an immune cell delivery potentiating lipid comprises a sterol. In another embodiment, an immune cell delivery potentiating lipid comprises a naturally occurring sterol. In another embodiment, an immune cell delivery potentiating lipid comprises a modified sterol. In one embodiment, an immune cell delivery potentiating lipid comprises one or more phytosterols. In one embodiment, the immune cell delivery potentiating lipid comprises a phytosterol/cholesterol blend.

In one embodiment, the immune cell delivery potentiating lipid comprises an effective amount of a phytosterol.

The term “phytosterol” refers to the group of plant based sterols and stanols that are phytosteroids including salts or esters thereof.

The term “sterol” refers to the subgroup of steroids also known as steroid alcohols. Sterols are usually divided into two classes: (1) plant sterols also known as “phytosterols”, and (2) animal sterols also known as “zoosterols” such as cholesterol. The term “stanol” refers to the class of saturated sterols, having no double bonds in the sterol ring structure.

The term “effective amount of phytosterol” is intended to mean an amount of one or more phytosterols in a lipid-based composition, including an LNP, that will elicit a desired activity (e.g., enhanced delivery, enhanced immune cell uptake, enhanced nucleic acid activity). In some embodiments, an effective amount of phytosterol is all or substantially all (i.e., about 99-100%) of the sterol in a lipid nanoparticle. In some embodiments, an effective amount of phytosterol is less than all or substantially all of the sterol in a lipid nanoparticle (less than about 99-100%), but greater than the amount of non-phytosterol sterol in the lipid nanoparticle. In some embodiments, an effective amount of phytosterol is greater than 50%, greater than 60%, greater than 70%, greater than 75%, greater than 80%, greater than 85%, greater than 90% or greater than 95% the total amount of sterol in a lipid nanoparticle. In some embodiments, an effective amount of phytosterol is 95-100%, 75-100%, or 50-100% of the total amount of sterol in a lipid nanoparticle.

In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, Δ5-avenaserol, Δ7-avenaserol or a Δ7-stigmasterol, including analogs, salts or esters thereof, alone or in combination. In some embodiments, the phytosterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol.

In some embodiments, the sitosterol is a beta-sitosterol.

In some embodiments, the beta-sitosterol has the formula:

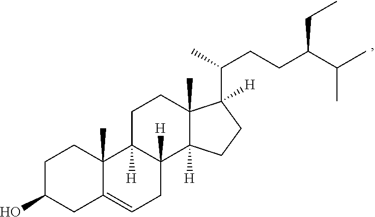

including analogs, salts or esters thereof.

In some embodiments, the sitosterol is a stigmasterol.

In some embodiments, the stigmasterol has the formula:

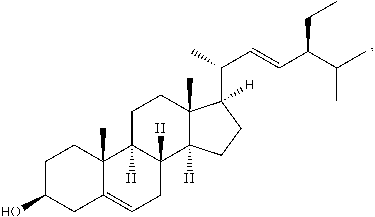

including analogs, salts or esters thereof.

In some embodiments, the sitosterol is a campesterol.

In some embodiments, the campesterol has the formula:

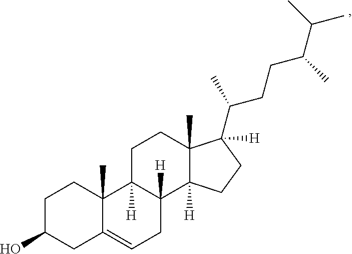

including analogs, salts or esters thereof.

In some embodiments, the sitosterol is a sitostanol.

In some embodiments, the sitostanol has the formula:

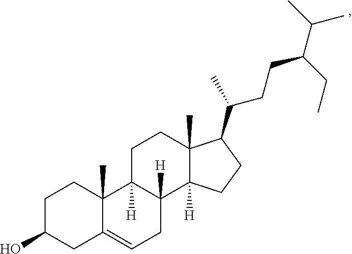

including analogs, salts or esters thereof.

In some embodiments, the sitosterol is a campestanol.

In some embodiments, the campestanol has the formula:

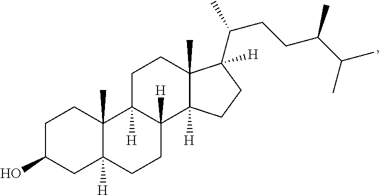

including analogs, salts or esters thereof.

In some embodiments, the sitosterol is a brassicasterol.

In some embodiments, the brassicasterol has the formula:

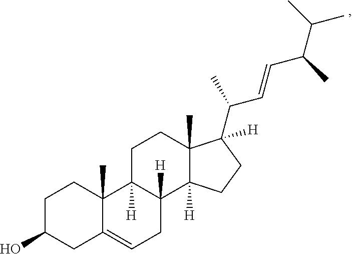

including analogs, salts or esters thereof.

In some embodiments, the sitosterol is a fucosterol.

In some embodiments, the fucosterol has the formula:

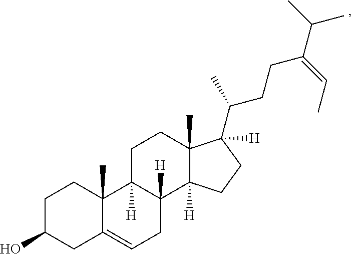

including analogs, salts or esters thereof.

In some embodiments, the phytosterol (e.g., beta-sitosterol) has a purity of greater than 70%. In some embodiments, the phytosterol (e.g., beta-sitosterol) has a purity of greater than 80%. In some embodiments, the phytosterol (e.g., beta-sitosterol) has a purity of greater than 90%. In some embodiments, the phytosterol (e.g., beta-sitosterol) has a purity of greater than 95%. In some embodiments, the phytosterol (e.g., beta-sitosterol) has a purity of greater than 97%, 98% or 99%.

In one embodiment, an immune cell delivery enhancing LNP comprises more than one type of structural lipid.

For example, in one embodiment, the immune cell delivery enhancing LNP comprises at least one immune cell delivery potentiating lipid which is a phytosterol. In one embodiment, the phytosterol is the only structural lipid present in the LNP. In another embodiment, the immune cell delivery LNP comprises a blend of structural lipids.

In one embodiment, the combined amount of the phytosterol and structural lipid (e.g., beta-sitosterol and cholesterol) in the lipid composition of a pharmaceutical composition disclosed herein ranges from about 20 mol % to about 60 mol %, from about 25 mol % to about 55 mol %, from about 30 mol % to about 50 mol %, or from about 35 mol % to about 45 mol %.

In one embodiment, the combined amount of the phytosterol and structural lipid (e.g., beta-sitosterol and cholesterol) in the lipid composition disclosed herein ranges from about 25 mol % to about 30 mol %, from about 30 mol % to about 35 mol %, or from about 35 mol % to about 40 mol %.

In one embodiment, the amount of the phytosterol and structural lipid (e.g., beta-sitosterol and cholesterol) in the lipid composition disclosed herein is about 24 mol %, about 29 mol %, about 34 mol %, or about 39 mol %.

In some embodiments, the combined amount of the phytosterol and structural lipid (e.g., beta-sitosterol and cholesterol) in the lipid composition disclosed herein is at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %.

In some embodiments, the lipid nanoparticle comprises one or more phytosterols (e.g., beta-sitosterol) and one or more structural lipids (e.g. cholesterol). In some embodiments, the mol % of the structural lipid is between about 1% and 50% of the mol % of phytosterol present in the lipid nanoparticle. In some embodiments, the mol % of the structural lipid is between about 10% and 40% of the mol % of phytosterol present in the lipid-based composition (e.g., LNP). In some embodiments, the mol % of the structural lipid is between about 20% and 30% of the mol % of phytosterol present in the lipid-based composition (e.g., LNP). In some embodiments, the mol % of the structural lipid is about 30% of the mol % of phytosterol present in the lipid-based composition (e.g., lipid nanoparticle).

In some embodiments, the lipid nanoparticle comprises between 15 and 40 mol % phytosterol (e.g., beta-sitosterol). In some embodiments, the lipid nanoparticle comprises about 15, 16, 17, 18, 19, 20, 21, 22, 23, 24, 25, 26, 27, 28, 29, 30, 31, 32, 33, 34, 35, 36, 37, 38, 30 or 40 mol % phytosterol (e.g., beta-sitosterol) and 0, 1, 2, 3, 4, 5, 6, 7, 8, 9, 10, 11, 12, 13, 14, 15, 16, 17, 18, 19, 20, 21, 22, 23, 24 or 25 mol % structural lipid (e.g., cholesterol). In some embodiments, the lipid nanoparticle comprises more than 20 mol % phytosterol (e.g., beta-sitosterol) and less than 20 mol % structural lipid (e.g., cholesterol), so that the total mol % of phytosterol and structural lipid is between 30 and 40 mol %. In some embodiments, the lipid nanoparticle comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 37 mol %, about 38 mol %, about 39 mol % or about 40 mol % phytosterol (e.g., beta-sitosterol); and about 19 mol %, about 18 mol % about 17 mol %, about 16 mol %, about 15 mol %, about 14 mol %, about 13 mol %, about 12 mol %, about 11 mol %, about 10 mol %, about 9 mol %, about 8 mol %, about 7 mol %, about 6 mol %, about 5 mol %, about 4 mol %, about 3 mol %, about 2 mol %, about 1 mol % or about 0 mol %, respectively, of a structural lipid (e.g., cholesterol). In some embodiments, the lipid nanoparticle comprises about 28 mol % phytosterol (e.g., beta-sitosterol) and about 10 mol % structural lipid (e.g., cholesterol). In some embodiments, the lipid nanoparticle comprises a total mol % of phytosterol and structural lipid (e.g., cholesterol) of 38.5%. In some embodiments, the lipid nanoparticle comprises 28.5 mol % phytosterol (e.g., beta-sitosterol) and 10 mol % structural lipid (e.g., cholesterol). In some embodiments, the lipid nanoparticle comprises 18.5 mol % phytosterol (e.g., beta-sitosterol) and 20 mol % structural lipid (e.g., cholesterol).

In certain embodiments, the LNP comprises 50% ionizable lipid, 10% helper lipid (e.g, phospholipid), 38.5% structural lipid, and 1.5% PEG lipid. In certain embodiments, the LNP comprises 50% ionizable lipid, 10% helper lipid (e.g, phospholipid), 38% structural lipid, and 2% PEG lipid. In certain embodiments, the LNP comprises 50% ionizable lipid, 20% helper lipid (e.g, phospholipid), 28.5% structural lipid, and 1.5% PEG lipid. In certain embodiments, the LNP comprises 50% ionizable lipid, 20% helper lipid (e.g, phospholipid), 28% structural lipid, and 2% PEG lipid. In certain embodiments, the LNP comprises 40% ionizable lipid, 30% helper lipid (e.g, phospholipid), 28.5% structural lipid, and 1.5% PEG lipid. In certain embodiments, the LNP comprises 40% ionizable lipid, 30% helper lipid (e.g, phospholipid), 28% structural lipid, and 2% PEG lipid. In certain embodiments, the LNP comprises 45% ionizable lipid, 20% helper lipid (e.g, phospholipid), 33.5% structural lipid, and 1.5% PEG lipid. In certain embodiments, the LNP comprises 45% ionizable lipid, 20% helper lipid (e.g, phospholipid), 33% structural lipid, and 2% PEG lipid.

In one aspect, the immune cell delivery enhancing LNP comprises phytosterol and the LNP does not comprise an additional structural lipid. Accordingly, the structural lipid (sterol) component of the LNP consists of phytosterol. In another aspect, the immune cell delivery enhancing LNP comprises phytosterol and an additional structural lipid. Accordingly, the sterol component of the LNP comprise phytosterol and one or more additional sterols or structural lipids.

In any of the foregoing or related aspects, the structural lipid (e.g., sterol, such as a phytosterol or phytosterol/cholesterol blend) of the LNP of the disclosure comprises a compound described herein as cholesterol, β-sitosterol (also referred to herein as Cmpd S 141), campesterol (also referred to herein as Cmpd S 143), β-sitostanol (also referred to herein as Cmpd S 144), brassicasterol or stigmasterol, or combinations or blends thereof. In another embodiment, the structural lipid (e.g., sterol, such as a phytosterol or phytosterol/cholesterol blend) of the LNP of the disclosure comprises a compound selected from cholesterol, β-sitosterol, campesterol, β-sitostanol, brassicasterol, stigmasterol, β-sitosterol-d7, Compound S-30, Compound S-31, Compound S-32, or combinations or blends thereof. In another embodiment, the structural lipid (e.g., sterol, such as a phytosterol or phytosterol/cholesterol blend) of the LNP of the disclosure comprises a compound described herein as cholesterol, β-sitosterol (also referred to herein as Cmpd S 141), campesterol (also referred to herein as Cmpd S 143), β-sitostanol (also referred to herein as Cmpd S 144), Compound S-140, Compound S-144, brassicasterol (also referred to herein as Cmpd S 148) or Composition S-183 (˜40% Compound S-141, ˜25% Compound S-140, ˜25% Compound S-143 and ˜10% brassicasterol). In some embodiments, the structural lipid of the LNP of the disclosure comprises a compound described herein as Compound S-159, Compound S-160, Compound S-164, Compound S-165, Compound S-167, Compound S-170, Compound S-173 or Compound S-175.

In one embodiment, an immune cell delivery enhancing LNP comprises a non-cationic helper lipid, e.g., phospholipid. In any of the foregoing or related aspects, the non-cationic helper lipid (e.g, phospholipid) of the LNP of the disclosure comprises a compound described herein as DSPC, DMPE, DOPC or H-409. In one embodiment, the non-cationic helper lipid, e.g., phospholipid is DSPC. In other embodiments, the non-cationic helper lipid (e.g., phospholipid) of the LNP of the disclosure comprises a compound described herein as DSPC, DMPE, DOPC, DPPC, PMPC, H-409, H-418, H-420, H-421 or H-422.

In any of the foregoing or related aspects, the PEG lipid of the LNP of the disclosure comprises a compound described herein can be selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In another embodiment, the PEG lipid is selected from the group consisting of Compound Nos. P415, P416, P417, P 419, P 420, P 423, P 424, P 428, P L5, P L1, P L2, P L16, P L17, P L18, P L19, P L22, P L23, DMG, DPG and DSG. In another embodiment, the PEG lipid is selected from the group consisting of Cmpd 428, PL16, PL17, PL 18, PL19, P L5, PL 1, and PL 2.

In one embodiment, an immune cell delivery potentiating lipid comprises an effective amount of a combination of an ionizable lipid and a phytosterol.

In other embodiments, the disclosure provides lipid nanoparticles comprising one or more immune cell delivery potentiating lipids, wherein the LNP comprises Compound X as the ionizable lipid, DSPC as the phospholipid, cholesterol or a cholesterol/β-sitosterol blend as the structural lipid and Compound 428 as the PEG lipid. In various embodiments of these Compound X-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2; For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol. In another embodiment, the structural lipid is cholesterol/β-sitosterol at a total percentage of 38.5%, wherein the blend can comprise, for example: (i) 20% cholesterol and 18.5% β-sitosterol; or (ii) 10% cholesterol and 28.5% β-sitosterol.

In other embodiments, the disclosure provides lipid nanoparticles comprising one or more immune cell delivery potentiating lipids, wherein the LNP comprises Compound Y as the ionizable lipid, DSPC as the phospholipid, cholesterol or a cholesterol/β-sitosterol blend as the structural lipid and Compound 428 as the PEG lipid. In various embodiments of these Compound Y-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-182 as the ionizable lipid, DSPC as the phospholipid, cholesterol or a cholesterol/β-sitosterol blend as the structural lipid and Compound 428 as the PEG lipid. In various embodiments of these Compound I-182-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-321 as the ionizable lipid, DSPC as the phospholipid, cholesterol or a cholesterol/β-sitosterol blend as the structural lipid and Compound 428 as the PEG lipid. In various embodiments of these Compound I-321-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-292 as the ionizable lipid, DSPC as the phospholipid, cholesterol or a cholesterol/β-sitosterol blend as the structural lipid and Compound 428 as the PEG lipid. In various embodiments of these Compound I-292-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-326 as the ionizable lipid, DSPC as the phospholipid, cholesterol or a cholesterol/β-sitosterol blend as the structural lipid and Compound 428 as the PEG lipid. In various embodiments of these Compound I-326-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-301 as the ionizable lipid, DSPC as the phospholipid, cholesterol or a cholesterol/β-sitosterol blend as the structural lipid and Compound 428 as the PEG lipid. In various embodiments of these Compound I-301-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-48 as the ionizable lipid, DSPC as the phospholipid, cholesterol or a cholesterol/β-sitosterol blend as the structural lipid and Compound 428 as the PEG lipid. In various embodiments of these Compound I-48-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-50 as the ionizable lipid, DSPC as the phospholipid, cholesterol or a cholesterol/β-sitosterol blend as the structural lipid and Compound 428 as the PEG lipid. In various embodiments of these Compound I-50-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-328 as the ionizable lipid, DSPC as the phospholipid, cholesterol or a cholesterol/β-sitosterol blend as the structural lipid and Compound 428 as the PEG lipid. In various embodiments of these Compound I-328-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-330 as the ionizable lipid, DSPC as the phospholipid, cholesterol or a cholesterol/β-sitosterol blend as the structural lipid and Compound 428 as the PEG lipid. In various embodiments of these Compound I-330-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-109 as the ionizable lipid, DSPC as the phospholipid, cholesterol or a cholesterol/β-sitosterol blend as the structural lipid and Compound 428 as the PEG lipid. In various embodiments of these Compound I-109-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-111 as the ionizable lipid, DSPC as the phospholipid, cholesterol or a cholesterol/β-sitosterol blend as the structural lipid and Compound 428 as the PEG lipid. In various embodiments of these Compound I-111-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-181 as the ionizable lipid, DSPC as the phospholipid, cholesterol or a cholesterol/β-sitosterol blend as the structural lipid and Compound 428 as the PEG lipid. In various embodiments of these Compound I-181-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises any of Compounds X, Y, I-321, I-292,1-326, 1-182, 1-301, 1-48, 1-50, 1-328, 1-330, 1-109, I-111 or 1-181 as the ionizable lipid; DSPC as the phospholipid; cholesterol, a cholesterol/β-sitosterol blend, a β-sitosterol/β-sitostanol blend, a β-sitosterol/camposterol blend, a β-sitosterol/β-sitostanol/camposterol blend, a cholesterol/camposterol blend, a cholesterol/β-sitostanol blend, a cholesteroal/β-sitostanol/camposterol blend or a cholesterol/β-sitosterol/β-sitostanol/camposterol blend as the structural lipid; and Compound 428 as the PEG lipid. In various embodiments of these compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2; (v) 40:18.5:40:1.5; or (vi) 45:20:33.5:1.5. In one embodiment, for the structural lipid component, the LNP can comprise, for example, 40% structural lipid composed of (i) 10% cholesterol and 30% f3-sitosterol; (ii) 10% cholesterol and 30% campesterol; (iii) 10% cholesterol and 30% β-sitostanol; (iv) 10% cholesterol, 20% β-sitosterol and 10% campesterol; (v) 10% cholesterol, 20% β-sitosterol and 10% β-sitostanol; (vi) 10% cholesterol, 10% β-sitosterol and 20% campesterol; (vii) 10% cholesterol, 10% β-sitosterol and 20% campesterol; (viii) 10% cholesterol, 20% campesterol and 10% β-sitostanol; (ix) 10% cholesterol, 10% campesterol and 20% β-sitostanol; or (x) 10% cholesterol, 10% β-sitosterol, 10% campesterol and 10% β-sitostanol. In another embodiment, for the structural lipid component, the LNP can comprise, for example, 33.5% structural lipid composed of (i) 33.5% cholesterol; (ii) 18.5% cholesterol, 15% β-sitosterol; (iii) 18.5% cholesterol, 15% campesterol; or (iv) 18.5% cholesterol, 15% campesterol.

In other embodiments, the disclosure provides lipid nanoparticles comprising one or more immune cell delivery potentiating lipids, wherein the LNP comprises Compound I-301, Compound I-321 or Compound I-326 as the ionizable lipid; DSPC as the phospholipid; cholesterol or a cholesterol/β-sitosterol blend as the structural lipid; and Compound 428 as the PEG lipid. In one embodiment, the LNP enhances delivery to T cells (e.g., CD3+ T cells).

In other embodiment, the disclosure provides lipid nanoparticles comprising one or more immune cell delivery potentiating lipids, wherein the LNP comprises Compound X, Compound 1-109, Compound I-111, Compound I-181, Compound I-182 or Compound I-244, wherein the LNP enhances delivery to monocytes. The other components of the LNP can be selected from those disclosed herein, for example DSPC as the phospholipid; cholesterol or a cholesterol/β-sitosterol blend as the structural lipid; and Compound 428 as the PEG lipid.

In other embodiment, the disclosure provides lipid nanoparticles comprising one or more immune cell delivery potentiating lipids, wherein the LNP comprises camposterol, β-sitostanol or stigmasterol as the structural lipid, wherein the LNP enhances delivery to monocytes. The other components of the LNP can be selected from those disclosed herein, for example Compound X, Compound I-109, Compound I-111, Compound I-181, Compound I-182 or Compound I-244 as the ionizable lipid; DSPC as the phospholipid; and Compound 428 as the PEG lipid.

In other embodiment, the disclosure provides lipid nanoparticles comprising one or more immune cell delivery potentiating lipids, wherein the LNP comprises DOPC, DMPE or H-409 as the helper lipid (e.g., phospholipid), wherein the LNP enhances delivery to monocytes. The other components of the LNP can be selected from those disclosed herein, for example Compound X, Compound I-109, Compound I-111, Compound I-181, Compound I-182 or Compound I-244 as the ionizable lipid; cholesterol, a cholesterol/β-sitosterol blend, camposterol, β-sitostanol or stigmasterol as the structural lipid; and Compound 428 as the PEG lipid.

### Exemplary Additional LNP Components

Surfactants

In certain embodiments, the lipid nanoparticles of the disclosure optionally includes one or more surfactants.

In certain embodiments, the surfactant is an amphiphilic polymer. As used herein, an amphiphilic “polymer” is an amphiphilic compound that comprises an oligomer or a polymer. For example, an amphiphilic polymer can comprise an oligomer fragment, such as two or more PEG monomer units. For example, an amphiphilic polymer described herein can be PS 20.

For example, the amphiphilic polymer is a block copolymer.

For example, the amphiphilic polymer is a lyoprotectant.

For example, amphiphilic polymer has a critical micelle concentration (CMC) of less than 2×10⁻⁴ M in water at about 30° C. and atmospheric pressure.

For example, amphiphilic polymer has a critical micelle concentration (CMC) ranging between about 0.1×10⁻⁴ M and about 1.3×10⁻⁴ M in water at about 30° C. and atmospheric pressure.

For example, the concentration of the amphiphilic polymer ranges between about its CMC and about 30 times of CMC (e.g., up to about 25 times, about 20 times, about 15 times, about 10 times, about 5 times, or about 3 times of its CMC) in the formulation, e.g., prior to freezing or lyophilization.

For example, the amphiphilic polymer is selected from poloxamers (Pluronic®), poloxamines (Tetronic®), polyoxyethylene glycol sorbitan alkyl esters (polysorbates) and polyvinyl pyrrolidones (PVPs).

For example, the amphiphilic polymer is a poloxamer. For example, the amphiphilic polymer is of the following structure:

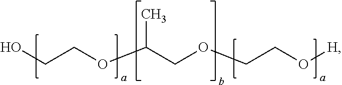

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

For example, the amphiphilic polymer is P124, P188, P237, P338, or P407.

For example, the amphiphilic polymer is P188 (e.g., Poloxamer 188, CAS Number 9003-11-6, also known as Kolliphor P188).

For example, the amphiphilic polymer is a poloxamine, e.g., tetronic 304 or tetronic 904.

For example, the amphiphilic polymer is a polyvinylpyrrolidone (PVP), such as PVP with molecular weight of 3 kDa, 10 kDa, or 29 kDa.

For example, the amphiphilic polymer is a polysorbate, such as PS 20.

In certain embodiments, the surfactant is a non-ionic surfactant.

In some embodiments, the lipid nanoparticle comprises a surfactant. In some embodiments, the surfactant is an amphiphilic polymer. In some embodiments, the surfactant is a non-ionic surfactant.

For example, the non-ionic surfactant is selected from the group consisting of polyethylene glycol ether (Brij), poloxamer, polysorbate, sorbitan, and derivatives thereof.

For example, the polyethylene glycol ether is a compound of Formula (VIII):

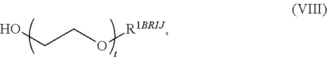

or a salt or isomer thereof, wherein:

t is an integer between 1 and 100;

R^{1BRU} independently is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NRNC(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and each instance of R^{N} is independently hydrogen, C₁-6 alkyl, or a nitrogen protecting group In some embodiment, R^{1BRU} is C₁₈ alkyl. For example, the polyethylene glycol ether is a compound of Formula (VIII-a):

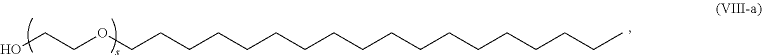

or a salt or isomer thereof.

In some embodiments, R^{1BRU} is C₁₈ alkenyl. For example, the polyethylene glycol ether is a compound of Formula (VIII-b):

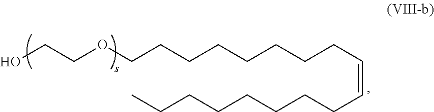

or a salt or isomer thereof

In some embodiments, the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

In some embodiments, the polysorbate is Tween® 20, Tween® 40, Tween®, 60, or Tween® 80.

In some embodiments, the derivative of sorbitan is Span® 20, Span® 60, Span® 65, Span® 80, or Span® 85.

In some embodiments, the concentration of the non-ionic surfactant in the lipid nanoparticle ranges from about 0.00001% w/v to about 1% w/v, e.g., from about 0.00005% w/v to about 0.5% w/v, or from about 0.0001% w/v to about 0.1% w/v.

In some embodiments, the concentration of the non-ionic surfactant in lipid nanoparticle ranges from about 0.000001 wt % to about 1 wt %, e.g., from about 0.000002 wt % to about 0.8 wt %, or from about 0.000005 wt % to about 0.5 wt %.

In some embodiments, the concentration of the PEG lipid in the lipid nanoparticle ranges from about 0.01% by molar to about 50% by molar, e.g., from about 0.05% by molar to about 20% by molar, from about 0.07% by molar to about 10% by molar, from about 0.1% by molar to about 8% by molar, from about 0.2% by molar to about 5% by molar, or from about 0.25% by molar to about 3% by molar.

Adjuvants

In some embodiments, an LNP of the invention optionally includes one or more adjuvants, e.g., Glucopyranosyl Lipid Adjuvant (GLA), CpG oligodeoxynucleotides (e.g., Class A or B), poly(I:C), aluminum hydroxide, and Pam3CSK4.

Other Components

An LNP of the invention may optionally include one or more components in addition to those described in the preceding sections. For example, a lipid nanoparticle may include one or more small hydrophobic molecules such as a vitamin (e.g., vitamin A or vitamin E) or a sterol.

Lipid nanoparticles may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a lipid nanoparticle. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A lipid nanoparticle may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy. 21s^{t} Edition, A. R. Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### LNP Compositions

A lipid nanoparticle described herein may be designed for one or more specific applications or targets. The elements of a lipid nanoparticle and their relative amounts may be selected based on a particular application or target, and/or based on the efficacy, toxicity, expense, ease of use, availability, or other feature of one or more elements. Similarly, the particular formulation of a lipid nanoparticle may be selected for a particular application or target according to, for example, the efficacy and toxicity of particular combinations of elements. The efficacy and tolerability of a lipid nanoparticle formulation may be affected by the stability of the formulation.

The LNPs of the invention comprise at least one immune cell delivery potentiating lipid. The subject LNPs comprise: an effective amount of an immune cell delivery potentiating lipid as a component of an LNP, wherein the LNP comprises an (i) ionizable lipid; (ii) cholesterol or other structural lipid; (iii) a non-cationic helper lipid or phospholipid; a (iv) PEG lipid and (v) an agent (e.g, an nucleic acid molecule) encapsulated in and/or associated with the LNP, wherein the effective amount of the immune cell delivery potentiating lipid enhances delivery of the agent to an immune cell (e.g., a human or primate immune cell) relative to an LNP lacking the immune cell delivery potentiating lipid.

The elements of the various components may be provided in specific fractions, e.g., mole percent fractions.

For example, in any of the foregoing or related aspects, the LNP of the disclosure comprises a structural lipid or a salt thereof. In some aspects, the structural lipid is cholesterol or a salt thereof. In further aspects, the mol % cholesterol is between about 1% and 50% of the mol % of phytosterol present in the LNP. In other aspects, the mol % cholesterol is between about 10% and 40% of the mol % of phytosterol present in the LNP. In some aspects, the mol % cholesterol is between about 20% and 30% of the mol % of phytosterol present in the LNP. In further aspects, the mol % cholesterol is about 30% of the mol % of phytosterol present in the LNP.

In any of the foregoing or related aspects, the LNP of the disclosure comprises about 30 mol % to about 60 mol % ionizable lipid, about 0 mol % to about 30 mol % phospholipid, about 18.5 mol % to about 48.5 mol % sterol, and about 0 mol % to about 10 m of % PEG lipid.

In any of the foregoing or related aspects, the LNP of the disclosure comprises about 35 mol % to about 55 mol % ionizable lipid, about 5 mol % to about 25 mol % phospholipid, about 30 mol % to about 40 mol % sterol, and about 0 mol % to about 10 mol % PEG lipid.

In any of the foregoing or related aspects, the LNP of the disclosure comprises about 50 mol % ionizable lipid, about 10 mol % phospholipid, about 38.5 mol % sterol, and about 1.5 mol % PEG lipid.

In certain embodiments, the ionizable lipid component of the lipid nanoparticle includes about 30 mol % to about 60 mol % ionizable lipid, about 0 mol % to about 30 mol % non-cationic helper lipid, about 18.5 mol % to about 48.5 mol % phytosterol optionally including one or more structural lipids, and about 0 mol % to about 10 mol % of PEG lipid, provided that the total mol % does not exceed 100%. In some embodiments, the ionizable lipid component of the lipid nanoparticle includes about 35 mol % to about 55 mol % ionizable lipid, about 5 mol % to about 25 mol % non-cationic helper lipid, about 30 mol % to about 40 mol % phytosterol optionally including one or more structural lipids, and about 0 mol % to about 10 mol % of PEG lipid. In a particular embodiment, the lipid component includes about 50 mol % ionizable lipid, about 10 mol % non-cationic helper lipid, about 38.5 mol % phytosterol optionally including one or more structural lipids, and about 1.5 mol % of PEG lipid. In another particular embodiment, the lipid component includes about 40 mol % ionizable lipid, about 20 mol % non-cationic helper lipid, about 38.5 mol % phytosterol optionally including one or more structural lipids, and about 1.5 mol % of PEG lipid. In some embodiments, the phytosterol may be beta-sitosterol, the non-cationic helper lipid may be a phospholipid such as DOPE, DSPC or a phospholipid substitute such as oleic acid. In other embodiments, the PEG lipid may be PEG-DMG and/or the structural lipid may be cholesterol.

In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol, and about 0 mol % to about 10 mol % PEG lipid. In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol and a structural lipid, and about 0 mol % to about 10 mol % PEG lipid. In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol and cholesterol, and about 0 mol % to about 10 mol % PEG lipid.

In some aspects, the LNP of the disclosure comprises about 35 mol % to about 55 mol % ionizable lipid, about 5 mol % to about 25 mol % non-cationic helper lipid, about 30 mol % to about 40 mol % phytosterol, and about 0 mol % to about 10 mol % PEG lipid. In some aspects, the LNP of the disclosure comprises about 35 mol % to about 55 mol % ionizable lipid, about 5 mol % to about 25 mol % non-cationic helper lipid, about 30 mol % to about 40 mol % phytosterol and a structural lipid, and about 0 mol % to about 10 mol % PEG lipid. In some aspects, the LNP of the disclosure comprises about 35 mol % to about 55 mol % ionizable lipid, about 5 mol % to about 25 mol % non-cationic helper lipid, about 30 mol % to about 40 mol % phytosterol and cholesterol, and about 0 mol % to about 10 mol % PEG lipid.

In some aspects, the LNP of the disclosure comprises about 50 mol % ionizable lipid, about 10 mol % non-cationic helper lipid, about 38.5 mol % phytosterol, and about 1.5 mol % PEG lipid. In some aspects, the LNP of the disclosure comprises about 50 mol % ionizable lipid, about 10 mol % non-cationic helper lipid, about 38.5 mol % phytosterol and a structural lipid, and about 1.5 mol % PEG lipid. In some aspects, the LNP of the disclosure comprises about 50 mol % ionizable lipid, about 10 mol % non-cationic helper lipid, about 38.5 mol % phytosterol and cholesterol, and about 1.5 mol % PEG lipid.

In some aspects, the LNP of the disclosure comprises about 40 mol % ionizable lipid, about 20 mol % non-cationic helper lipid, about 38.5 mol % phytosterol, and about 1.5 mol % PEG lipid. In some aspects, the LNP of the disclosure comprises about 40 mol % ionizable lipid, about 20 mol % non-cationic helper lipid, about 38.5 mol % phytosterol and a structural lipid, and about 1.5 mol % PEG lipid. In some aspects, the LNP of the disclosure comprises about 40 mol % ionizable lipid, about 20 mol % non-cationic helper lipid, about 38.5 mol % phytosterol and cholesterol, and about 1.5 mol % PEG lipid.

In some aspects, the LNP of the disclosure comprises about 45 mol % ionizable lipid, about 10 mol % non-cationic helper lipid, about 38.5 mol % phytosterol, and about 1.5 mol % PEG lipid. In some aspects, the LNP of the disclosure comprises about 45 mol % ionizable lipid, about 10 mol % non-cationic helper lipid, about 38.5 mol % phytosterol and a structural lipid, and about 1.5 mol % PEG lipid. In some aspects, the LNP of the disclosure comprises about 45 mol % ionizable lipid, about 10 mol % non-cationic helper lipid, about 38.5 mol % phytosterol and cholesterol, and about 1.5 mol % PEG lipid.

In some aspects, the LNP of the disclosure comprises about 55 mol % ionizable lipid, about 5 mol % non-cationic helper lipid, about 38.5 mol % phytosterol, and about 1.5 mol % PEG lipid. In some aspects, the LNP of the disclosure comprises about 55 mol % ionizable lipid, about 5 mol % non-cationic helper lipid, about 38.5 mol % phytosterol and a structural lipid, and about 1.5 mol % PEG lipid. In some aspects, the LNP of the disclosure comprises about 55 mol % ionizable lipid, about 5 mol % non-cationic helper lipid, about 38.5 mol % phytosterol and cholesterol, and about 1.5 mol % PEG lipid.

In some aspects, the LNP of the disclosure comprises about 60 mol % ionizable lipid, about 5 mol % non-cationic helper lipid, about 33.5 mol % phytosterol, and about 1.5 mol % PEG lipid. In some aspects, the LNP of the disclosure comprises about 60 mol % ionizable lipid, about 5 mol % non-cationic helper lipid, about 33.5 mol % phytosterol and a structural lipid, and about 1.5 mol % PEG lipid. In some aspects, the LNP of the disclosure comprises about 60 mol ionizable lipid, about 5 mol % non-cationic helper lipid, about 33.5 mol % phytosterol and cholesterol, and about 1.5 mol % PEG lipid.

In some aspects, the LNP of the disclosure comprises about 45 mol % ionizable lipid, about 20 mol % non-cationic helper lipid, about 33.5 mol % phytosterol, and about 1.5 mol % PEG lipid. In some aspects, the LNP of the disclosure comprises about 45 mol % ionizable lipid, about 20 mol % non-cationic helper lipid, about 33.5 mol % phytosterol and a structural lipid, and about 1.5 mol % PEG lipid. In some aspects, the LNP of the disclosure comprises about 45 mol % ionizable lipid, about 20 mol % non-cationic helper lipid, about 33.5 mol % phytosterol and cholesterol, and about 1.5 mol % PEG lipid.

In some aspects, the LNP of the disclosure comprises about 50 mol % ionizable lipid, about 20 mol % non-cationic helper lipid, about 28.5 mol % phytosterol, and about 1.5 mol % PEG lipid. In some aspects, the LNP of the disclosure comprises about 50 mol % ionizable lipid, about 20 mol % non-cationic helper lipid, about 28.5 mol % phytosterol and a structural lipid, and about 1.5 mol % PEG lipid. In some aspects, the LNP of the disclosure comprises about 50 mol % ionizable lipid, about 20 mol % non-cationic helper lipid, about 28.5 mol % phytosterol and cholesterol, and about 1.5 mol % PEG lipid.

In some aspects, the LNP of the disclosure comprises about 55 mol % ionizable lipid, about 20 mol % non-cationic helper lipid, about 23.5 mol % phytosterol, and about 1.5 mol % PEG lipid. In some aspects, the LNP of the disclosure comprises about 55 mol % ionizable lipid, about 20 mol % non-cationic helper lipid, about 23.5 mol % phytosterol and a structural lipid, and about 1.5 mol % PEG lipid. In some aspects, the LNP of the disclosure comprises about 55 mol % ionizable lipid, about 20 mol % non-cationic helper lipid, about 23.5 mol % phytosterol and cholesterol, and about 1.5 mol % PEG lipid.

In some aspects, the LNP of the disclosure comprises about 60 mol % ionizable lipid, about 20 mol % non-cationic helper lipid, about 18.5 mol % phytosterol, and about 1.5 mol % PEG lipid. In some aspects, the LNP of the disclosure comprises about 60 mol % ionizable lipid, about 20 mol % non-cationic helper lipid, about 18.5 mol % phytosterol and a structural lipid, and about 1.5 mol % PEG lipid. In some aspects, the LNP of the disclosure comprises about 60 mol % ionizable lipid, about 20 mol % non-cationic helper lipid, about 18.5 mol % phytosterol and cholesterol, and about 1.5 mol % PEG lipid.

In some aspects, the LNP of the disclosure comprises about 40 mol % ionizable lipid, about 15 mol % non-cationic helper lipid, about 43.5 mol % phytosterol, and about 1.5 mol % PEG lipid. In some aspects, the LNP of the disclosure comprises about 40 mol % ionizable lipid, about 15 mol % non-cationic helper lipid, about 43.5 mol % phytosterol and a structural lipid, and about 1.5 mol % PEG lipid. In some aspects, the LNP of the disclosure comprises about 40 mol % ionizable lipid, about 15 mol % non-cationic helper lipid, about 43.5 mol % phytosterol and cholesterol, and about 1.5 mol % PEG lipid.

In some aspects, the LNP of the disclosure comprises about 50 mol % ionizable lipid, about 15 mol % non-cationic helper lipid, about 33.5 mol % phytosterol, and about 1.5 mol % PEG lipid. In some aspects, the LNP of the disclosure comprises about 50 mol % ionizable lipid, about 15 mol % non-cationic helper lipid, about 33.5 mol % phytosterol and a structural lipid, and about 1.5 mol % PEG lipid. In some aspects, the LNP of the disclosure comprises about 50 mol % ionizable lipid, about 15 mol % non-cationic helper lipid, about 33.5 mol % phytosterol and cholesterol, and about 1.5 mol % PEG lipid.

In some aspects, the LNP of the disclosure comprises about 55 mol % ionizable lipid, about 15 mol % non-cationic helper lipid, about 28.5 mol % phytosterol, and about 1.5 mol % PEG lipid. In some aspects, the LNP of the disclosure comprises about 55 mol % ionizable lipid, about 15 mol % non-cationic helper lipid, about 28.5 mol % phytosterol and a structural lipid, and about 1.5 mol % PEG lipid. In some aspects, the LNP of the disclosure comprises about 55 mol % ionizable lipid, about 15 mol % non-cationic helper lipid, about 28.5 mol % phytosterol and cholesterol, and about 1.5 mol % PEG lipid.

In some aspects, the LNP of the disclosure comprises about 60 mol % ionizable lipid, about 15 mol % non-cationic helper lipid, about 23.5 mol % phytosterol, and about 1.5 mol % PEG lipid. In some aspects, the LNP of the disclosure comprises about 60 mol % ionizable lipid, about 15 mol % non-cationic helper lipid, about 23.5 mol % phytosterol and a structural lipid, and about 1.5 mol % PEG lipid. In some aspects, the LNP of the disclosure comprises about 60 mol % ionizable lipid, about 15 mol % non-cationic helper lipid, about 23.5 mol % phytosterol and cholesterol, and about 1.5 mol % PEG lipid.

In some aspects, the LNP of the disclosure comprises about 40 mol % ionizable lipid, about 10 mol % non-cationic helper lipid, about 48.5 mol % phytosterol, and about 1.5 mol % PEG lipid. In some aspects, the LNP of the disclosure comprises about 40 mol % ionizable lipid, about 10 mol % non-cationic helper lipid, about 48.5 mol % phytosterol and a structural lipid, and about 1.5 mol % PEG lipid. In some aspects, the LNP of the disclosure comprises about 40 mol % ionizable lipid, about 10 mol % non-cationic helper lipid, about 48.5 mol % phytosterol and cholesterol, and about 1.5 mol % PEG lipid.

In some aspects, the LNP of the disclosure comprises about 45 mol % ionizable lipid, about 10 mol % non-cationic helper lipid, about 43.5 mol % phytosterol, and about 1.5 mol % PEG lipid. In some aspects, the LNP of the disclosure comprises about 45 mol % ionizable lipid, about 10 mol % non-cationic helper lipid, about 43.5 mol % phytosterol and a structural lipid, and about 1.5 mol % PEG lipid. In some aspects, the LNP of the disclosure comprises about 45 mol % ionizable lipid, about 10 mol % non-cationic helper lipid, about 43.5 mol % phytosterol and cholesterol, and about 1.5 mol % PEG lipid.

In some aspects, the LNP of the disclosure comprises about 55 mol % ionizable lipid, about 10 mol % non-cationic helper lipid, about 33.5 mol % phytosterol, and about 1.5 mol % PEG lipid. In some aspects, the LNP of the disclosure comprises about 55 mol % ionizable lipid, about 10 mol % non-cationic helper lipid, about 33.5 mol % phytosterol and a structural lipid, and about 1.5 mol % PEG lipid. In some aspects, the LNP of the disclosure comprises about 55 mol % ionizable lipid, about 10 mol % non-cationic helper lipid, about 33.5 mol % phytosterol and cholesterol, and about 1.5 mol % PEG lipid.

In some aspects, the LNP of the disclosure comprises about 60 mol % ionizable lipid, about 10 mol % non-cationic helper lipid, about 28.5 mol % phytosterol, and about 1.5 mol % PEG lipid. In some aspects, the LNP of the disclosure comprises about 60 mol % ionizable lipid, about 10 mol % non-cationic helper lipid, about 28.5 mol % phytosterol and a structural lipid, and about 1.5 mol % PEG lipid. In some aspects, the LNP of the disclosure comprises about 60 mol % ionizable lipid, about 10 mol % non-cationic helper lipid, about 28.5 mol % phytosterol and cholesterol, and about 1.5 mol % PEG lipid.

In some aspects, the LNP of the disclosure comprises about 40 mol % ionizable lipid, about 5 mol % non-cationic helper lipid, about 53.5 mol % phytosterol, and about 1.5 mol % PEG lipid. In some aspects, the LNP of the disclosure comprises about 40 mol % ionizable lipid, about 5 mol % non-cationic helper lipid, about 53.5 mol % phytosterol and a structural lipid, and about 1.5 mol % PEG lipid. In some aspects, the LNP of the disclosure comprises about 40 mol ionizable lipid, about 5 mol % non-cationic helper lipid, about 53.5 mol % phytosterol and cholesterol, and about 1.5 mol % PEG lipid.

In some aspects, the LNP of the disclosure comprises about 45 mol % ionizable lipid, about 5 mol % non-cationic helper lipid, about 48.5 mol % phytosterol, and about 1.5 mol % PEG lipid. In some aspects, the LNP of the disclosure comprises about 45 mol % ionizable lipid, about 5 mol % non-cationic helper lipid, about 48.5 mol % phytosterol and a structural lipid, and about 1.5 mol % PEG lipid. In some aspects, the LNP of the disclosure comprises about 45 mol % ionizable lipid, about 5 mol % non-cationic helper lipid, about 48.5 mol % phytosterol and cholesterol, and about 1.5 mol % PEG lipid.

In some aspects, the LNP of the disclosure comprises about 50 mol % ionizable lipid, about 5 mol % non-cationic helper lipid, about 43.5 mol % phytosterol, and about 1.5 mol % PEG lipid. In some aspects, the LNP of the disclosure comprises about 50 mol % ionizable lipid, about 5 mol % non-cationic helper lipid, about 43.5 mol % phytosterol and a structural lipid, and about 1.5 mol % PEG lipid. In some aspects, the LNP of the disclosure comprises about 50 mol % ionizable lipid, about 5 mol % non-cationic helper lipid, about 43.5 mol % phytosterol and cholesterol, and about 1.5 mol % PEG lipid.

In some aspects, the LNP of the disclosure comprises about 40 mol % ionizable lipid, about 20 mol % non-cationic helper lipid, about 40 mol % phytosterol, and about 0 mol % PEG lipid. In some aspects, the LNP of the disclosure comprises about 40 mol % ionizable lipid, about 20 mol % non-cationic helper lipid, about 40 mol % phytosterol and a structural lipid, and about 0 mol % PEG lipid. In some aspects, the LNP of the disclosure comprises about 40 mol % ionizable lipid, about 20 mol % non-cationic helper lipid, about 40 mol % phytosterol and cholesterol, and about 0 mol % PEG lipid.

In some aspects, the LNP of the disclosure comprises about 45 mol % ionizable lipid, about 20 mol % non-cationic helper lipid, about 35 mol % phytosterol, and about 0 mol % PEG lipid. In some aspects, the LNP of the disclosure comprises about 45 mol % ionizable lipid, about 20 mol % non-cationic helper lipid, about 35 mol % phytosterol and a structural lipid, and about 0 mol % PEG lipid. In some aspects, the LNP of the disclosure comprises about 45 mol % ionizable lipid, about 20 mol % non-cationic helper lipid, about 35 mol % phytosterol and cholesterol, and about 0 mol % PEG lipid.

In some aspects, the LNP of the disclosure comprises about 50 mol % ionizable lipid, about 20 mol % non-cationic helper lipid, about 30 mol % phytosterol, and about 0 mol % PEG lipid. In some aspects, the LNP of the disclosure comprises about 50 mol % ionizable lipid, about 20 mol % non-cationic helper lipid, about 30 mol % phytosterol and a structural lipid, and about 0 mol % PEG lipid. In some aspects, the LNP of the disclosure comprises about 50 mol % ionizable lipid, about 20 mol % non-cationic helper lipid, about 30 mol % phytosterol and cholesterol, and about 0 mol % PEG lipid.

In some aspects, the LNP of the disclosure comprises about 55 mol % ionizable lipid, about 20 mol % non-cationic helper lipid, about 25 mol % phytosterol, and about 0 mol % PEG lipid. In some aspects, the LNP of the disclosure comprises about 55 mol % ionizable lipid, about 20 mol % non-cationic helper lipid, about 25 mol % phytosterol and a structural lipid, and about 0 mol % PEG lipid. In some aspects, the LNP of the disclosure comprises about 55 mol % ionizable lipid, about 20 mol % non-cationic helper lipid, about 25 mol % phytosterol and cholesterol, and about 0 mol % PEG lipid.

In some aspects, the LNP of the disclosure comprises about 60 mol % ionizable lipid, about 20 mol % non-cationic helper lipid, about 20 mol % phytosterol, and about 0 mol % PEG lipid. In some aspects, the LNP of the disclosure comprises about 60 mol % ionizable lipid, about 20 mol % non-cationic helper lipid, about 20 mol % phytosterol and a structural lipid, and about 0 mol % PEG lipid. In some aspects, the LNP of the disclosure comprises about 60 mol % ionizable lipid, about 20 mol % non-cationic helper lipid, about 20 mol % phytosterol and cholesterol, and about 0 mol % PEG lipid.

In some aspects, the LNP of the disclosure comprises about 40 mol % ionizable lipid, about 15 mol % non-cationic helper lipid, about 45 mol % phytosterol, and about 0 mol % PEG lipid. In some aspects, the LNP of the disclosure comprises about 40 mol % ionizable lipid, about 15 mol % non-cationic helper lipid, about 45 mol % phytosterol and a structural lipid, and about 0 mol % PEG lipid. In some aspects, the LNP of the disclosure comprises about 40 mol % ionizable lipid, about 15 mol % non-cationic helper lipid, about 45 mol % phytosterol and cholesterol, and about 0 mol % PEG lipid.

In some aspects, the LNP of the disclosure comprises about 45 mol % ionizable lipid, about 15 mol % non-cationic helper lipid, about 40 mol % phytosterol, and about 0 mol % PEG lipid. In some aspects, the LNP of the disclosure comprises about 45 mol % ionizable lipid, about 15 mol % non-cationic helper lipid, about 40 mol % phytosterol and a structural lipid, and about 0 mol % PEG lipid. In some aspects, the LNP of the disclosure comprises about 45 mol % ionizable lipid, about 15 mol % non-cationic helper lipid, about 40 mol % phytosterol and cholesterol, and about 0 mol % PEG lipid.

In some aspects, the LNP of the disclosure comprises about 50 mol % ionizable lipid, about 15 mol % non-cationic helper lipid, about 35 mol % phytosterol, and about 0 mol % PEG lipid. In some aspects, the LNP of the disclosure comprises about 50 mol % ionizable lipid, about 15 mol % non-cationic helper lipid, about 35 mol % phytosterol and a structural lipid, and about 0 mol % PEG lipid. In some aspects, the LNP of the disclosure comprises about 50 mol % ionizable lipid, about 15 mol % non-cationic helper lipid, about 35 mol % phytosterol and cholesterol, and about 0 mol % PEG lipid.

In some aspects, the LNP of the disclosure comprises about 55 mol % ionizable lipid, about 15 mol % non-cationic helper lipid, about 30 mol % phytosterol, and about 0 mol % PEG lipid. In some aspects, the LNP of the disclosure comprises about 55 mol % ionizable lipid, about 15 mol % non-cationic helper lipid, about 30 mol % phytosterol and a structural lipid, and about 0 mol % PEG lipid. In some aspects, the LNP of the disclosure comprises about 55 mol % ionizable lipid, about 15 mol % non-cationic helper lipid, about 30 mol % phytosterol and cholesterol, and about 0 mol % PEG lipid.

In some aspects, the LNP of the disclosure comprises about 60 mol % ionizable lipid, about 15 mol % non-cationic helper lipid, about 25 mol % phytosterol, and about 0 mol % PEG lipid. In some aspects, the LNP of the disclosure comprises about 60 mol % ionizable lipid, about 15 mol % non-cationic helper lipid, about 25 mol % phytosterol and a structural lipid, and about 0 mol % PEG lipid. In some aspects, the LNP of the disclosure comprises about 60 mol % ionizable lipid, about 15 mol % non-cationic helper lipid, about 25 mol % phytosterol and cholesterol, and about 0 mol % PEG lipid.

In some aspects, the LNP of the disclosure comprises about 40 mol % ionizable lipid, about 10 mol % non-cationic helper lipid, about 50 mol % phytosterol, and about 0 mol % PEG lipid. In some aspects, the LNP of the disclosure comprises about 40 mol % ionizable lipid, about 10 mol % non-cationic helper lipid, about 50 mol % phytosterol and a structural lipid, and about 0 mol % PEG lipid. In some aspects, the LNP of the disclosure comprises about 40 mol % ionizable lipid, about 10 mol % non-cationic helper lipid, about 50 mol % phytosterol and cholesterol, and about 0 mol % PEG lipid.

In some aspects, the LNP of the disclosure comprises about 45 mol % ionizable lipid, about 10 mol % non-cationic helper lipid, about 45 mol % phytosterol, and about 0 mol % PEG lipid. In some aspects, the LNP of the disclosure comprises about 45 mol % ionizable lipid, about 10 mol % non-cationic helper lipid, about 45 mol % phytosterol and a structural lipid, and about 0 mol % PEG lipid. In some aspects, the LNP of the disclosure comprises about 45 mol % ionizable lipid, about 10 mol % non-cationic helper lipid, about 45 mol % phytosterol and cholesterol, and about 0 mol % PEG lipid.

In some aspects, the LNP of the disclosure comprises about 50 mol % ionizable lipid, about 0 mol % non-cationic helper lipid, about 48.5 mol % phytosterol, and about 1.5 mol % PEG lipid. In some aspects, the LNP of the disclosure comprises about 50 mol % ionizable lipid, about 0 mol % non-cationic helper lipid, about 48.5 mol % phytosterol and a structural lipid, and about 1.5 mol % PEG lipid. In some aspects, the LNP of the disclosure comprises about 50 mol % ionizable lipid, about 0 mol % non-cationic helper lipid, about 48.5 mol % phytosterol and cholesterol, and about 1.5 mol % PEG lipid.

In some aspects, the LNP of the disclosure comprises about 50 mol % ionizable lipid, about 10 mol % non-cationic helper lipid, about 40 mol % phytosterol, and about 0 mol % PEG lipid. In some aspects, the LNP of the disclosure comprises about 50 mol % ionizable lipid, about 10 mol % non-cationic helper lipid, about 40 mol % phytosterol and a structural lipid, and about 0 mol % PEG lipid. In some aspects, the LNP of the disclosure comprises about 50 mol % ionizable lipid, about 10 mol % non-cationic helper lipid, about 40 mol % phytosterol and cholesterol, and about 0 mol % PEG lipid.

In some aspects, the LNP of the disclosure comprises about 55 mol % ionizable lipid, about 10 mol % non-cationic helper lipid, about 35 mol % phytosterol, and about 0 mol % PEG lipid. In some aspects, the LNP of the disclosure comprises about 55 mol % ionizable lipid, about 10 mol % non-cationic helper lipid, about 35 mol % phytosterol and a structural lipid, and about 0 mol % PEG lipid. In some aspects, the LNP of the disclosure comprises about 55 mol % ionizable lipid, about 10 mol % non-cationic helper lipid, about 35 mol % phytosterol and cholesterol, and about 0 mol % PEG lipid.

In some aspects, the LNP of the disclosure comprises about 60 mol % ionizable lipid, about 10 mol % non-cationic helper lipid, about 30 mol % phytosterol, and about 0 mol % PEG lipid. In some aspects, the LNP of the disclosure comprises about 60 mol % ionizable lipid, about 10 mol % non-cationic helper lipid, about 30 mol % phytosterol and a structural lipid, and about 0 mol % PEG lipid. In some aspects, the LNP of the disclosure comprises about 60 mol % ionizable lipid, about 10 mol % non-cationic helper lipid, about 30 mol % phytosterol and cholesterol, and about 0 mol % PEG lipid.

In some aspects with respect to the embodiments herein, the phytosterol and a structural lipid components of a LNP of the disclosure comprises between about 10:1 and 1:10 phytosterol to structural lipid, such as about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9 and 1:10 phytosterol to structural lipid (e.g. beta-sitosterol to cholesterol).

In some embodiments, the phytosterol component of the LNP is a blend of the phytosterol and a structural lipid, such as cholesterol, wherein the phytosterol (e.g., beta-sitosterol) and the structural lipid (e.g., cholesterol) are each present at a particular mol %. For example, in some embodiments, the lipid nanoparticle comprises between 15 and 40 mol % phytosterol (e.g., beta-sitosterol). In some embodiments, the lipid nanoparticle comprises about 15, 16, 17, 18, 19, 20, 21, 22, 23, 24, 25, 26, 27, 28, 29, 30, 31, 32, 33, 34, 35, 36, 37, 38, 30 or 40 mol % phytosterol (e.g., beta-sitosterol) and 0, 1, 2, 3, 4, 5, 6, 7, 8, 9, 10, 11, 12, 13, 14, 15, 16, 17, 18, 19, 20, 21, 22, 23, 24 or 25 mol % structural lipid (e.g., cholesterol). In some embodiments, the lipid nanoparticle comprises more than 20 mol % phytosterol (e.g., beta-sitosterol) and less than 20 mol % structural lipid (e.g., cholesterol), so that the total mol % of phytosterol and structural lipid is between 30 and 40 mol %. In some embodiments, the lipid nanoparticle comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 37 mol %, about 38 mol %, about 39 mol % or about 40 mol % phytosterol (e.g., beta-sitosterol); and about 19 mol %, about 18 mol % about 17 mol %, about 16 mol %, about 15 mol %, about 14 mol %, about 13 mol %, about 12 mol %, about 11 mol %, about 10 mol %, about 9 mol %, about 8 mol %, about 7 mol %, about 6 mol %, about 5 mol %, about 4 mol %, about 3 mol %, about 2 mol %, about 1 mol % or about 0 mol %, respectively, of a structural lipid (e.g., cholesterol). In some embodiments, the lipid nanoparticle comprises about 28 mol % phytosterol (e.g., beta-sitosterol) and about 10 mol % structural lipid (e.g., cholesterol). In some embodiments, the lipid nanoparticle comprises a total mol % of phytosterol and structural lipid (e.g., cholesterol) of 38.5%. In some embodiments, the lipid nanoparticle comprises 28.5 mol % phytosterol (e.g., beta-sitosterol) and 10 mol % structural lipid (e.g., cholesterol). In some embodiments, the lipid nanoparticle comprises 18.5 mol % phytosterol (e.g., beta-sitosterol) and 20 mol % structural lipid (e.g., cholesterol).

Lipid nanoparticles of the disclosure may be designed for one or more specific applications or targets. For example, the subject lipid nanoparticles may optionally be designed to further enhance delivery of a nucleic acid molecule, such as an RNA, to a particular immune cell (e.g., lymphoid cell or myeloid cell), tissue, organ, or system or group thereof in a mammal's, e.g., a human's body. Physiochemical properties of lipid nanoparticles may be altered in order to increase selectivity for particular bodily targets. For instance, particle sizes may be adjusted to promote immune cell uptake. As set forth above, the nucleic acid molecule included in a lipid nanoparticle may also be selected based on the desired delivery to immune cells. For example, a nucleic acid molecule may be selected for a particular indication, condition, disease, or disorder and/or for delivery to a particular cell, tissue, organ, or system or group thereof (e.g., localized or specific delivery).

In certain embodiments, a lipid nanoparticle may include an mRNA encoding a polypeptide of interest capable of being translated within a cell to produce a polypeptide of interest. In other embodiments, the lipid nanoparticle can include other types of agents, such as other nucleic acid agents, including DNA and/or RNA agents, as described herein, e.g., siRNAs, miRNAs, antisense nucleic acid and the like as described in further detail below.

The amount of a nucleic acid molecule in a lipid nanoparticle may depend on the size, composition, desired target and/or application, or other properties of the lipid nanoparticle as well as on the properties of the therapeutic and/or prophylactic. For example, the amount of an RNA useful in a lipid nanoparticle may depend on the size, sequence, and other characteristics of the RNA. The relative amounts of a nucleic acid molecule and other elements (e.g., lipids) in a lipid nanoparticle may also vary. In some embodiments, the wt/wt ratio of the ionizable lipid component to a nucleic acid molecule, in a lipid nanoparticle may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the ionizable lipid component to a nucleic acid molecule may be from about 10:1 to about 40:1. In certain embodiments, the wt/wt ratio is about 20:1. The amount of a nucleic acid molecule in a LNP may, for example, be measured using absorption spectroscopy (e.g., ultraviolet-visible spectroscopy).

In some embodiments, a lipid nanoparticle includes one or more RNAs, and one or more ionizable lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 5.7:1, about 5.8:1, about 5.9:1, about 6.0:1, about 6.5:1, or about 7.0:1. For example, the N:P ratio may be about 5.67:1. In another embodiment, the N:P ratio may be about 5.8:1.

In some embodiments, the formulation including a lipid nanoparticle may further includes a salt, such as a chloride salt.

In some embodiments, the formulation including a lipid nanoparticle may further includes a sugar such as a disaccharide. In some embodiments, the formulation further includes a sugar but not a salt, such as a chloride salt.

### Physical Properties

The characteristics of a lipid nanoparticle may depend on the components thereof. For example, a lipid nanoparticle including cholesterol as a structural lipid may have different characteristics than a lipid nanoparticle that includes a different structural lipid. Similarly, the characteristics of a lipid nanoparticle may depend on the absolute or relative amounts of its components. For instance, a lipid nanoparticle including a higher molar fraction of a phospholipid may have different characteristics than a lipid nanoparticle including a lower molar fraction of a phospholipid. Characteristics may also vary depending on the method and conditions of preparation of the lipid nanoparticle.

Lipid nanoparticles may be characterized by a variety of methods. For example, microscopy (e.g., transmission electron microscopy or scanning electron microscopy) may be used to examine the morphology and size distribution of a lipid nanoparticle. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a lipid nanoparticle, such as particle size, polydispersity index, and zeta potential.

The mean size of a lipid nanoparticle may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a lipid nanoparticle may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a lipid nanoparticle may be from about 70 nm to about 100 nm. In a particular embodiment, the mean size may be about 80 nm. In other embodiments, the mean size may be about 100 nm.

A lipid nanoparticle may be relatively homogenous. A polydispersity index may be used to indicate the homogeneity of a LNP, e.g., the particle size distribution of the lipid nanoparticles. As used herein, the “polydispersity index” is a ratio that describes the homogeneity of the particle size distribution of a system. A small value, e.g., less than 0.3, indicates a narrow particle size distribution. A small (e.g., less than 0.3) polydispersity index generally indicates a narrow particle size distribution. A lipid nanoparticle may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a lipid nanoparticle may be from about 0.10 to about 0.20.

The zeta potential of a lipid nanoparticle may be used to indicate the electrokinetic potential of the composition. As used herein, the “zeta potential” is the electrokinetic potential of a lipid, e.g., in a particle composition.

For example, the zeta potential may describe the surface charge of a lipid nanoparticle. Lipid nanoparticles with relatively low charges, positive or negative, are generally desirable, as more highly charged species may interact undesirably with cells, tissues, and other elements in the body. In some embodiments, the zeta potential of a lipid nanoparticle may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a a nucleic acid molecule describes the amount of nucleic acid molecule that is encapsulated or otherwise associated with a lipid nanoparticle after preparation, relative to the initial amount provided. The encapsulation efficiency is desirably high (e.g., close to 100%). The encapsulation efficiency may be measured, for example, by comparing the amount of nucleic acid molecule in a solution containing the lipid nanoparticle before and after breaking up the lipid nanoparticle with one or more organic solvents or detergents. Fluorescence may be used to measure the amount of free nucleic acid molecules (e.g., RNA) in a solution. For the lipid nanoparticles described herein, the encapsulation efficiency of a nucleic acid molecule may be at least 50%, for example 50%, 55%, 60%, 65%, 70%, 75%, 80%, 85%, 90%, 91%, 92%, 93%, 94%, 95%, 96%. 97%, 98%, 99%, or 100%. In some embodiments, the encapsulation efficiency may be at least 80%. In certain embodiments, the encapsulation efficiency may be at least 90%.

A lipid nanoparticle may optionally comprise one or more coatings. For example, a lipid nanoparticle may be formulated in a capsule, film, or tablet having a coating. A capsule, film, or tablet including a composition described herein may have any useful size, tensile strength, hardness, or density.

### Uses of Lipid-Based Compositions

The present disclosure provides improved lipid-based compositions, in particular LNP compositions, with enhanced delivery of nucleic acids to immune cells. The present disclosure is based, at least in part, on the discovery that components of LNPs, act as immune cell delivery potentiating lipids that enhance delivery of an encapsulated nucleic acid molecule (e.g., an mRNA) to immune cells, such as lymphoid cells and myeloid cells (e.g., T cells, B cells, monocytes and dendritic cells).

The improved lipid-based compositions of the disclosure, in particular LNPs, are useful for a variety of purposes, both in vitro and in vivo, such as for nucleic acid delivery to immune cells, protein expression in or on immune cells, modulating immune cell (e.g., T cell, B cell, monocyte, and/or dendritic cell) activation or activity, increasing an immune response to a protein (e.g., infectious disease or cancer antigen) of interest (e.g., for vaccination or therapeutic purposes) and decreasing immune cell responses to reduce autoimmunity (e.g., to tolerize T cells).

For in vitro protein expression, the immune cell is contacted with the LNP by incubating the LNP and the immune cell ex vivo. Such immune cells may subsequently be introduced in vivo.

For in vivo protein expression, the immune cell is contacted with the LNP by administering the LNP to a subject to thereby increase or induce protein expression in or on immune cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

For in vitro delivery, in one embodiment the immune cell is contacted with the LNP by incubating the LNP and the immune cell ex vivo. In one embodiment, the immune cell is a human immune cell. In another embodiment, the immune cell is a primate immune cell. In another embodiment, the immune cell is a human or non-human primate immune cell. Various types of immune cells have been demonstrated to be transfectable by the LNP (see e.g., Examples 3, 5, 6, 9 and 10). In one embodiment, the immune cell is a T cell (e.g., a CD3+ T cell, a CD4+ T cell, a CD8+ T cell or a CD4+CD25+CD127^{low} Treg cell). In one embodiment, the immune cell is a B cell (e.g., a CD19+ B cell). In one embodiment, the immune cell is a dendritic cell (e.g., a CD11c+CD11b− dendritic cell). In one embodiment, the immune cell is a monocyte/macrophage (e.g., a CD11c-CD11b+ monocyte/macrophage). In one embodiment, the immune cell is an immature NK cell (e.g., a CD56^{HIGH} immature NK cell). In one embodiment, the immune cell is an activated NK cell (e.g., a CD56^{DIM} activated NK cell). In one embodiment, the immune cell is an NK T cell (e.g., a CD3+CD56+ NK T cell). In one embodiment, the immune cell is an AML leukemia cell (e.g., a CD33+ AML cell). In one embodiment, the immune cell is a bone marrow plasma cell (e.g., CD45+CD38+CD138+CD19+CD20-bone marrow plasma cell).

In one embodiment, the immune cell is contacted with the LNP in the presence of serum or C₁q for at least 15 minutes, which has been shown to be sufficient time for transfection of the cells ex vivo (see Example 13). In another embodiment, the immune cell is contacted with the LNP for, e.g., at least 30 minutes, at least 1 hour, at least 2 hours, at least 3 hours, at least 4 hours, at least 5 hours, at least 6 hours, at least 12 hours or at least 24 hours.

In one embodiment, the immune cell is contacted with the LNP for a single treatment/transfection. In another embodiment, the immune cell is contacted with the LNP for multiple treatments/transfections (e.g., two, three, four or more treatments/transfections of the same cells). Repeat transfection of the same cells has been demonstrated to lead to a dose-related increase in the percentage of cells transfected and in the level of expression of a protein encoded by the transfected nucleic acid without impacting cell viability (see Example 12).

In another embodiment, for in vivo delivery, the immune cell is contacted with the LNP by administering the LNP to a subject to thereby deliver the nucleic acid to immune cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

In one embodiment, an intracellular concentration of the nucleic acid molecule in the immune cell is enhanced. In one embodiment, an activity of the nucleic acid molecule in the immune cell is enhanced. In one embodiment, expression of the nucleic acid molecule in the immune cell is enhanced. In on embodiment, the nucleic acid molecule modulates the activation or activity of the immune cell. In one embodiment, the nucleic acid molecule increases the activation or activity of the immune cell. In one embodiment, the nucleic acid molecule decreases the activation or activity of the immune cell.

In certain embodiments, delivery of a nucleic acid to an immune cell by the immune cell delivery potentiating lipid-containing LNP results in delivery to a detectable amount of immune cells (e.g., delivery to a certain percentage of immune cells), e.g., in vivo following administration to a subject. In some embodiments, the immune cell delivery potentiating lipid containing LNP does not include a targeting moiety for immune cells (e.g., does not include an antibody with specificity for an immune cell marker, or a receptor ligand which targets the LNP to immune cells). For example, in one embodiment, administration of the immune cell delivery potentiating lipid-containing LNP results in delivery of the nucleic acid to at least about 15% of splenic T cells in vivo after a single intravenous injection (e.g., in a mouse model such as described in Example 15 or in a non-human primate model such as described in Example 8). In another embodiment, administration of the immune cell delivery potentiating lipid-containing LNP results in delivery of the nucleic acid to at least about 15%-25% of splenic B cells in vivo after a single intravenous injection (e.g., in a mouse model such as described in Example 15 or in a non-human primate model such as described in Example 8). In another embodiment, administration of the immune cell delivery potentiating lipid-containing LNP results in delivery of the nucleic acid to at least about 35%-40% of splenic dendritic cells in vivo after a single intravenous injection (e.g., in a mouse model such as described in Example 15 or in a non-human primate model such as described in Example 8). In another embodiment, administration of the immune cell delivery potentiating lipid-containing LNP results in delivery of the nucleic acid to at least about 5%-20% of bone marrow cells (femur and/or humerus) in vivo after a single intravenous injection (e.g., in a mouse model such as described in Example 15 or in a non-human primate model such as described in Example 8). The levels of delivery demonstrated herein make in vivo immune therapy possible.

In one embodiment, uptake of the nucleic acid molecule by the immune cell is enhanced. Uptake can be determined by methods known to one of skill in the art. For example, association/binding and/or uptake/internalization may be assessed using a detectably labeled, such as fluorescently labeled, LNP and tracking the location of such LNP in or on immune cells following various periods of incubation. In addition, mathematical models, such as the ordinary differential equation (ODE)-based model described by Radu Mihaila, et al., (Molecular Therapy: Nucleic Acids, Vol. 7: 246-255, 2017; herein incorporated by reference), allow for quantitation of delivery and uptake.

In another embodiment, function or activity of a nucleic acid molecule can be used as an indication of the delivery of the nucleic acid molecule. For example, in the case of siRNA, reduction in protein expression in a certain proportion of immune cells can be measured to indicate delivery of the siRNA to that proportion of cells. Similarly, in the case of mRNA, increase in protein expression in a certain proportion of immune cells can be measured to indicate delivery of the siRNA to that proportion of cells. One of skill in the art will recognize various ways to measure delivery of other nucleic acid molecules to immune cells.

In certain embodiments, the nucleic acid delivered to the immune cell encodes a protein of interest. Accordingly, in one embodiment, an activity of a protein of interest encoded by the nucleic acid molecule in the immune cell is enhanced. In one embodiment, expression of a protein encoded by the nucleic acid molecule in the immune cell is enhanced. In one embodiment, the protein modulates the activation or activity of the immune cell. In one embodiment, the protein increases the activation or activity of the immune cell. In one embodiment, the protein decreases the activation or activity of the immune cell.

In one embodiment, various agents can be used to label cells (e.g., T cell, B cell, monocyte, or dendritic cell) to measure delivery to that specific immune cell population. For example, the LNP can encapsulate a reporter nucleic acid (e.g., an mRNA encoding a detectable reporter protein), wherein expression of the reporter nucleic acid results in labeling of the cell population to which the reporter nucleic acid is delivered. Non-limiting examples of detectable reporter proteins include enhanced green fluorescent protein (EGFP) and luciferase.

Delivery of the nucleic acid to the immune cell by the immune cell delivery potentiating lipid-containing LNP can be measured in vitro or in vivo by, for example, detecting expression of a protein encoded by the nucleic acid associated with/encapsulated by the LNP or by detecting an effect (e.g., a biological effect) mediated by the nucleic acid associated with/encapsulated by the LNP. For protein detection, the protein can be, for example, a cell surface protein that is detectable, for example, by immunofluorescence or flow cytometry using an antibody that specifically binds the cell surface protein. Alternatively, a reporter nucleic acid encoding a detectable reporter protein can be used and expression of the reporter protein can be measured by standard methods known in the art.

Methods of the disclosure are useful to deliver nucleic acid molecules to a variety of immune cell types, including normal immune cells and malignant immune cells. In one embodiment, the immune cell is selected from the group consisting of T cells, dendritic cells, monocytes and B cells.

The methods can be used to deliver nucleic acid to immune cells located, for example, in the spleen, in the peripheral blood and/or in the bone marrow. In one embodiment, the immune cell is an immune progenitor cell. In one embodiment, the immune cell is a T cell. T cells can be identified by expression of one or more T cell markers known in the art, typically CD3. Additional T cell markers include CD4 or CD8. In one embodiment, the immune cell is a B cell. B cells can be identified by expression of one or more B cell markers known in the art, typically CD19. Additional B cell markers include CD24 and CD72. In one embodiment, the immune cell is a monocyte and/or a tissue macrophage. Monocytes and/or macrophages can be identified by expression of one or more monocyte and/or macrophage markers known in the art, such as CD2, CD11b, CD14 and/or CD16. In one embodiment, the immune cell is a dendritic cell. Dendritic cells can be identified by expression of one or more dendritic cell markers known in the art, typically CD11c. Additional dendritic cell markers include BDCA-1 and/or CD103.

The methods of the disclosure are useful to deliver nucleic acid molecules to bone marrow cells (e.g., in vivo), including immune cells within the bone marrow as well as other bone marrow cells, such as hematopoietic stem cells, immune cell precursers and fibroblasts. In one embodiment, the immune cells are bone marrow plasma cells. In one embodiment, the immune cells are bone marrow monocytes. In one embodiment, the cells are bone marrow early progenitor cells (e.g., proerythroblasts, monoblasts/myeloblasts and/or mesenchymal stem cells).

In one embodiment, the immune cell is a malignant cell, a cancer cell, e.g., as demonstrated by deregulated control of G1 progression. In one embodiment, the immune cell is a T cell that is malignant, cancerous or that exhibits deregulated control of G1 progression. In one embodiment, the immune cell is a leukemia cell or lymphoma cell. In one embodiment, the immune cell is an acute myelocytic leukemia (AML) cell. In other embodiments, the immune cell is a B cell that is malignant, e.g., a Hodgkins lymphoma cell, a non-Hodgkin's lymphoma cell, an anaplastic large cell lymphoma cell, a precursor-T lymphoblastic lymphoma, a follicular lymphoma cell, a small lymphocytic lymphoma cell, a marginal zone lymphoma cell, a diffuse large B cell lymphoma cell, a mantle cell lymphoma cell, a Burkitt's lymphoma cell, an acute lymphoblastic leukemia cell or a chronic lymphocytic leukemia cell.

The improved lipid-based compositions, including LNPs of the disclosure are useful to deliver more than one nucleic acid molecules to an immune cell or different populations of immune cells, by for example, administration of two or more different LNPs. In one embodiment, the method of the disclosure comprises contacting the immune cell (or administering to a subject), concurrently or consecutively, a first LNP and a second LNP, wherein the first and second LNP encapsulate the same or different nucleic acid molecules, wherein the first and second LNP include a phytosterol as a component. In other embodiments, the method of the disclosure comprises contacting the immune cell (or administering to a subject), concurrently or consecutively, a first LNP and a second LNP, wherein the first and second LNP encapsulate the same or different nucleic acid molecules, wherein the first LNP includes a phytosterol as a component and the second LNP lacks a phytosterol.

### Pharmaceutical Compositions

Formulations comprising lipid nanoparticles of the invention may be formulated in whole or in part as pharmaceutical compositions. Pharmaceutical compositions may include one or more lipid nanoparticles. For example, a pharmaceutical composition may include one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients may be used in any pharmaceutical composition, except insofar as any conventional excipient or accessory ingredient may be incompatible with one or more components of a LNP in the formulation of the disclosure. An excipient or accessory ingredient may be incompatible with a component of a LNP of the formulation if its combination with the component or LNP may result in any undesirable biological effect or otherwise deleterious effect.

A lipid nanoparticle of the disclosure formulated into a pharmaceutical composition can encapsulate a single nucleic acid or multiple nucleic acids. When encapsulating multiple nucleic acids, the nucleic acids can be of the same type (e.g., all mRNA) or can be of different types (e.g., mRNA and DNA). Furthermore, multiple LNPs can be formulated into the same or separate pharmaceutical compositions. For example, the same or separate pharmaceutical compositions can comprise a first LNP and a second LNP, wherein the first and second LNP encapsulate the same or different nucleic acid molecules, wherein the first and second LNP include an immune cell delivery potentiating lipid as a component. In other embodiments, the same or separate pharmaceutical compositions can comprise a first LNP and a second LNP, wherein the first and second LNP encapsulate the same or different nucleic acid molecules, wherein the first LNP includes a immune cell delivery potentiating lipid as a component and the second LNP lacks a immune cell delivery potentiating lipid.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

Lipid nanoparticles and/or pharmaceutical compositions including one or more lipid nanoparticles may be administered to any patient or subject, including those patients or subjects that may benefit from a therapeutic effect provided by the delivery of a therapeutic and/or prophylactic to one or more particular cells, tissues, organs, or systems or groups thereof, such as the renal system. Although the descriptions provided herein of lipid nanoparticles and pharmaceutical compositions including lipid nanoparticles are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more lipid nanoparticles may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., lipid nanoparticle). The amount of the active ingredient is generally equal to the dosage of the active ingredient which would be administered to a subject and/or a convenient fraction of such a dosage such as, for example, one-half or one-third of such a dosage.

Pharmaceutical compositions may be prepared in a variety of forms suitable for a variety of routes and methods of administration. In one embodiment, such compositions are prepared in liquid form or are lyophylized (e.g., and stored at 4° C. or below freezing). For example, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutics and/or prophylactics, additional agents such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (wt/wt) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 Such a formulation is administered in the manner in which snuff is taken, i.e. by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

### Definitions

As used herein, the term “T cell anergy” refers to a mechanism of peripheral tolerance whereby a T cell enters a hyporesponsive state established upon recognition of antigen in the absence of co-stimulation. Under such conditions, T cells fail to become fully activated and enter a state of unresponsiveness that prevents cell proliferation and cytokine production in response to antigen re-encounter.

“Autoantigens,” as used herein, are normal tissue constituents in the body targeted by an autologous humoral (B cell) or T cell mediated immune response that often results in damage to the tissue and/or autoimmune disease. “Autologous” as used herein refers to cells or tissues derived from the same individual or cells or tissues that are immunologically compatible, e.g., have an identical MHC/HLA haplotypes.

An “autoimmune disorder,” as used herein, refers to a disease state in which, via the action of white blood cells (e.g., B cells, T cells, macrophages, monocytes, or dendritic cells), a pathological immune response (e.g., pathological in duration and/or magnitude) against one or more endogenous antigens, i.e., one or more autoantigens, with consequent tissue damage that may result from direct attack on the cells bearing the one or more autoantigens, from immune-complex formation, or from local inflammation. Autoimmune diseases are characterized by increased inflammation due to immune system activation against self-antigens.

The terms “allograft”, “homograft” and “allogeneic graft” refer to the transplant of an organ or tissue from one individual to another of the same species with a different genotype, including transplants from cadaveric, living related, and living unrelated donors. A graft transplanted from one individual to the same individual is referred to as an “autologous graft” or “autograft”. A graft transplanted between two genetically identical or syngeneic individuals is referred to as a “syngeneic graft”. A graft transplanted between individuals of different species is referred to as a “xenogeneic graft” or “xenograft”.

As used herein the phrase “immune response” or its equivalent “immunological response” refers to the development of a cellular (mediated by antigen-specific T cells or their secretion products) directed against an autoantigen or an related epitope of an autoantigen. A cellular immune response is elicited by the presentation of polypeptide epitopes in association with Class I or Class II MHC molecules, to activate antigen-specific CD4+ T helper cells and/or CD8+ cytotoxic T cells. The response may also involve activation of other components.

As used herein, the term “immune cell” refers to cells that play a role in the immune response, including lymphocytes, such as B cells and T cells; natural killer cells; myeloid cells, such as monocytes, macrophages, eosinophils, mast cells, basophils, and granulocytes.

An “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+ or CD8+ T cell, or the inhibition of a Treg cell.

“Immunotherapy” refers to the treatment of a subject afflicted with, or at risk of contracting or suffering a recurrence of, a disease by a method comprising inducing, enhancing, suppressing or otherwise modifying an immune response.

A human “at risk of developing an autoimmune disorder” refers to a human with a family history of autoimmune disorders (e.g., a genetic predisposition to one or more inflammatory disorders) or one exposed to one or more autoimmune disorder/autoantibody-inducing conditions. For example, a human exposed to a shiga toxin is at risk for developing typical HUS. Humans with certain cancers (e.g., liquid tumors such as multiple myeloma or chronic lymphocytic leukemia) can pre-dispose patients to developing certain autoimmune hemolytic diseases. For example, PCH can follow a variety of infections (e.g., syphilis) or neoplasms such as non-Hodgkin's lymphoma. In another example, CAD can be associated with HIV infection, Mycoplasma pneumonia infection, non-Hodgkin's lymphoma, or Waldenstrom's macroglobulinemia. In yet another example, autoimmune hemolytic anemia is a well-known complication of human chronic lymphocytic leukemia, approximately 11% of CLL patients with advanced disease will develop AIHA. As many as 30% of CLL may be at risk for developing AIHA. See, e.g., Diehl et al. (1998) Semin Oncol 25(1):80-97 and Gupta et al. (2002) Leukemia 16(10):2092-2095.

A human “suspected of having an autoimmune disorder” is one who presents with one or more symptoms of an autoimmune disorder. Symptoms of autoimmune disorders can vary in severity and type with the particular autoimmune disorder and include, but are not limited to, redness, swelling (e.g., swollen joints), joints that are warm to the touch, joint pain, stiffness, loss of joint function, fever, chills, fatigue, loss of energy, pain, fever, pallor, icterus, urticarial dermal eruption, hemoglobinuria, hemoglobinemia, and anemia (e.g., severe anemia), headaches, loss of appetite, muscle stiffness, insomnia, itchiness, stuffy nose, sneezing, coughing, one or more neurologic symptoms such as dizziness, seizures, or pain. From the above it will be clear that not all humans are “suspected of having an autoimmune disorder.”

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a LNP, “about” may mean +/−5% of the recited value. For instance, a LNP including a lipid component having about 40% of a given compound may include 30-50% of the compound. In another example, delivery to at least about 15% of T cells may include delivery to 10-20% of T cells.

Cancer: As used herein, “cancer” is a condition involving abnormal and/or unregulated cell growth, e.g., a cell having deregulated control of G1 progression. Exemplary non-limiting cancers include adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colorectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, myelodysplastic syndrome (including refractory anemias and refractory cytopenias), myeloproliferative neoplasms or diseases (including polycythemia vera, essential thrombocytosis and primary myelofibrosis), liver cancer (e.g., hepatocellular carcinoma), non-small cell lung cancer, small cell lung cancer, lung carcinoid tumor, lymphoma of the skin, malignant mesothelioma, multiple myeloma, myelodysplasia syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment. In particular embodiments, the cancer is liver cancer (e.g., hepatocellular carcinoma) or colorectal cancer. In other embodiments, the cancer is a blood-based cancer or a hematopoetic cancer.

Conjugated.′ As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering a therapeutic and/or prophylactic to a subject may involve administering a LNP including the therapeutic and/or prophylactic to the subject (e.g., by an intravenous, intramuscular, intradermal, or subcutaneous route). Administration of a LNP to a mammal or mammalian cell may involve contacting one or more cells with the lipid nanoparticle.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the disclosure may be encapsulated in a lipid nanoparticle, e.g., a liposome.

Encapsulation efficiency: As used herein, “encapsulation efficiency” refers to the amount of a therapeutic and/or prophylactic that becomes part of a LNP, relative to the initial total amount of therapeutic and/or prophylactic used in the preparation of a LNP. For example, if 97 mg of therapeutic and/or prophylactic are encapsulated in a LNP out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Enhanced delivery: As used herein, the term “enhanced delivery” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a nucleic acid (e.g., a therapeutic and/or prophylactic mRNA) by a nanoparticle to a target cell of interest (e.g., immune cell) compared to the level of delivery of the nucleic acid (e.g., a therapeutic and/or prophylactic mRNA) by a control nanoparticle to a target cell of interest (e.g., immune cell). For example, “enhanced delivery” by a immune cell delivery potentiating lipid-containing LNP of the disclosure can be evaluated by comparison to the same LNP lacking an immune cell delivery potentiating lipid. The level of delivery of an immune cell delivery potentiating lipid-containing LNP to a particular cell (e.g., immune cell) may be measured by comparing the amount of protein produced in target cells using the phytoserol-containing LNP versus the same LNP lacking the immune cell delivery potentiating lipid (e.g., by mean fluorescence intensity using flow cytometry), comparing the % of target cells transfected using the immune cell delivery potentiating lipid-containing LNP versus the same LNP lacking the immune cell delivery potentiating lipid (e.g., by quantitative flow cytometry), or comparing the amount of therapeutic and/or prophylactic in target cells in vivo using the immune cell delivery potentiating lipid-containing LNP versus the same LNP lacking the immune cell delivery potentiating lipid. It will be understood that the enhanced delivery of a nanoparticle to a target cell need not be determined in a subject being treated, it may be determined in a surrogate such as an animal model (e.g., a mouse or non-human primate model). For example, for determining enhanced delivery to immune cells, a mouse or NHP model (e.g., as described in the Examples) can be used and delivery of an mRNA encoding a protein of interest by a immune cell delivery potentiating lipid-containing LNP can be evaluated in immune cells (e.g., from spleen, peripheral blood and/or bone marrow) (e.g., flow cytometry, fluorescence microscopy and the like) as compared to the same LNP lacking the immune cell delivery potentiating lipid.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a immune cell delivery potentiating lipid in a lipid composition (e.g., LNP) of the disclosure, an effective amount of a immune cell delivery potentiating lipid is an amount sufficient to effect a beneficial or desired result as compared to a lipid composition (e.g., LNP) lacking the immune cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering an immune cell delivery potentiating lipid-containing lipid nanoparticle such that an effective amount of lipid nanoparticles are taken up by immune cells in a subject, an effective amount of immune cell delivery potentiating lipid-containing LNP is an amount sufficient to effect a beneficial or desired result as compared to an LNP lacking the immune cell delivery potentiating lipid. Non-limiting examples of beneficial or desired results in the subject include increasing the percentage of cells transfected, increasing the level of expression of a protein encoded by a nucleic acid associated with/encapsulated by the immune cell delivery potentiating lipid-containing LNP and/or increasing a prophylactic or therapeutic effect in vivo of a nucleic acid, or its encoded protein, associated with/encapsulated by the immune cell delivery potentiating lipid-containing LNP, as compared to an LNP lacking the immune cell delivery potentiating lipid. In some embodiments, a therapeutically effective amount of immune cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of immune cells. For example, an effective amount of immune cell delivery potentiating lipid-containing LNP can be an amount that results in transfection of at least 5%, 10% or 15% of splenic T cells, at least 5%, 10%, 15%, 20% or 25% of splenic B cells, at least 5%, 10%, 15%, 20%, 25%, 30%, 35% or 40% of splenic dendritic cells and/or at least 5% of total bone marrow cells in a mouse model (e.g., as described in Example 9) after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques. A fragment of a protein can be, for example, a portion of a protein that includes one or more functional domains such that the fragment of the protein retains the functional activity of the protein.

GC-rich: As used herein, the term “GC-rich” refers to the nucleobase composition of a polynucleotide (e.g., mRNA), or any portion thereof (e.g., an RNA element), comprising guanine (G) and/or cytosine (C) nucleobases, or derivatives or analogs thereof, wherein the GC-content is greater than about 50%. The term “GC-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises about 50% GC-content. In some embodiments of the disclosure, GC-rich polynucleotides, or any portions thereof, are exclusively comprised of guanine (G) and/or cytosine (C) nucleobases.

GC-content: As used herein, the term “GC-content” refers to the percentage of nucleobases in a polynucleotide (e.g., mRNA), or a portion thereof (e.g., an RNA element), that are either guanine (G) and cytosine (C) nucleobases, or derivatives or analogs thereof, (from a total number of possible nucleobases, including adenine (A) and thymine (T) or uracil (U), and derivatives or analogs thereof, in DNA and in RNA). The term “GC-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof.

Heterologous: As used herein, “heterologous” indicates that a sequence (e.g., an amino acid sequence or the polynucleotide that encodes an amino acid sequence) is not normally present in a given polypeptide or polynucleotide. For example, an amino acid sequence that corresponds to a domain or motif of one protein may be heterologous to a second protein.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC, where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof. (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.)

Leaky scanning: A phenomenon known as “leaky scanning” can occur whereby the PIC bypasses the initiation codon and instead continues scanning downstream until an alternate or alternative initiation codon is recognized. Depending on the frequency of occurrence, the bypass of the initiation codon by the PIC can result in a decrease in translation efficiency. Furthermore, translation from this downstream AUG codon can occur, which will result in the production of an undesired, aberrant translation product that may not be capable of eliciting the desired therapeutic response. In some cases, the aberrant translation product may in fact cause a deleterious response (Kracht et al., (2017) Nat Med 23(4):501-507).

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Metastasis: As used herein, the term “metastasis” means the process by which cancer spreads from the place at which it first arose as a primary tumor to distant locations in the body. A secondary tumor that arose as a result of this process may be referred to as “a metastasis.”

Modified: As used herein “modified” or “modification” refers to a changed state or a change in composition or structure of a polynucleotide (e.g., mRNA). Polynucleotides may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof).

Modified: As used herein “modified” refers to a changed state or structure of a molecule of the disclosure. Molecules may be modified in many ways including chemically, structurally, and functionally. In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 mn. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, a-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside/Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from cancer (e.g., liver cancer or colorectal cancer).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.). Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

Pre-Initiation Complex (PIC): As used herein, the term “pre-initiation complex” (alternatively “43S pre-initiation complex”; abbreviated as “PIC”) refers to a ribonucleoprotein complex comprising a 40S ribosomal subunit, eukaryotic initiation factors (eIF1, eIF1A, eIF3, eIF5), and the eIF2-GTP-Met-tRNAᵢ^{Met} ternary complex, that is intrinsically capable of attachment to the 5′ cap of an mRNA molecule and, after attachment, of performing ribosome scanning of the 5′ UTR.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Residence time: As used herein, the term “residence time” refers to the time of occupancy of a pre-initiation complex (PIC) or a ribosome at a discrete position or location along an mRNA molecule.

Specific delivery: As used herein, the term “specific delivery,” “specifically deliver,” or “specifically delivering” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target cell of interest (e.g., mammalian immune cell) compared to an off-target cell (e.g., non-immune cells). The level of delivery of a nanoparticle to a particular cell may be measured by comparing the amount of protein produced in target cells versus non-target cells (e.g., by mean fluorescence intensity using flow cytometry, comparing the % of target cells versus non-target cells expressing the protein (e.g., by quantitative flow cytometry), comparing the amount of protein produced in a target cell versus non-target cell to the amount of total protein in said target cells versus non-target cell, or comparing the amount of therapeutic and/or prophylactic in a target cell versus non-target cell to the amount of total therapeutic and/or prophylactic in said target cell versus non-target cell. It will be understood that the ability of a nanoparticle to specifically deliver to a target cell need not be determined in a subject being treated, it may be determined in a surrogate such as an animal model (e.g., a mouse or NHP model). For example, for determining specific delivery to immune cells, a mouse or NHP model (e.g., as described in the Examples) can be used and delivery of an mRNA encoding a protein of interest can be evaluated in immune cells (e.g., from spleen, peripheral blood and/or bone marrow) as compared to non-immune cells by standard methods (e.g., flow cytometry, fluorescence microscopy and the like).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeted cells: As used herein, “targeted cells” refers to any one or more cells of interest. The cells may be found in vitro, in vivo, in situ, or in the tissue or organ of an organism. The organism may be an animal, preferably a mammal, more preferably a human and most preferably a patient. Target immune cells include, for example, CD3+ T cells, CD19+ B cells and CD11c+ dendritic cells, as well as monocytes, tissue macrophages, and bone marrow cells (including immune cells within bone marrow, hematopoietic stem cells, immune cell precursors and fibroblasts).

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning. Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Tumor: As used herein, a “tumor” is an abnormal growth of tissue, whether benign or malignant.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine-Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

A “high uridine codon” is defined as a codon comprising two or three uridines, a “low uridine codon” is defined as a codon comprising one uridine, and a “no uridine codon” is a codon without any uridines. In some embodiments, a uridine-modified sequence comprises substitutions of high uridine codons with low uridine codons, substitutions of high uridine codons with no uridine codons, substitutions of low uridine codons with high uridine codons, substitutions of low uridine codons with no uridine codons, substitution of no uridine codons with low uridine codons, substitutions of no uridine codons with high uridine codons, and combinations thereof. In some embodiments, a high uridine codon can be replaced with another high uridine codon. In some embodiments, a low uridine codon can be replaced with another low uridine codon. In some embodiments, a no uridine codon can be replaced with another no uridine codon. A uridine-modified sequence can be uridine enriched or uridine rarefied.

Uridine Enriched: As used herein, the terms “uridine enriched” and grammatical variants refer to the increase in uridine content (expressed in absolute value or as a percentage value) in a sequence optimized nucleic acid (e.g., a synthetic mRNA sequence) with respect to the uridine content of the corresponding candidate nucleic acid sequence. Uridine enrichment can be implemented by substituting codons in the candidate nucleic acid sequence with synonymous codons containing less uridine nucleobases. Uridine enrichment can be global (i.e., relative to the entire length of a candidate nucleic acid sequence) or local (i.e., relative to a subsequence or region of a candidate nucleic acid sequence).

Uridine Rarefied: As used herein, the terms “uridine rarefied” and grammatical variants refer to a decrease in uridine content (expressed in absolute value or as a percentage value) in an sequence optimized nucleic acid (e.g., a synthetic mRNA sequence) with respect to the uridine content of the corresponding candidate nucleic acid sequence. Uridine rarefication can be implemented by substituting codons in the candidate nucleic acid sequence with synonymous codons containing less uridine nucleobases. Uridine rarefication can be global (i.e., relative to the entire length of a candidate nucleic acid sequence) or local (i.e., relative to a subsequence or region of a candidate nucleic acid sequence).

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### OTHER EMBODIMENTS

The disclosure relates to the following embodiments. Throughout this section, the term embodiment is abbreviated as ‘E’ followed by an ordinal. For example, E1 is equivalent to Embodiment 1.

E1. An mRNA comprising:

(i) a 5′UTR

(ii) an open reading frame (ORF) encoding a polypeptide of interest; and

(iii) a 3′UTR,

wherein the 5′UTR, 3′UTR, or both comprise at least one microRNA (miR)-binding site targeted by a miR, wherein the miR is differentially expressed in a target immune cell relative to a plurality of non-target immune cells, such that the mRNA is selectively degraded in the target immune cell relative to the plurality of non-target immune cells.

E2. The mRNA of embodiment 1, wherein the target immune cell and the plurality of non-target immune cells are human immune cells.

E3. The mRNA of any one of claim 1 or 2, wherein the target immune cell and the plurality of non-target immune cell are of different immune cell types.

E4. The mRNA of embodiment 3, wherein the plurality of non-target immune cells comprises between 2 and 5 different immune cell types.

E5. The mRNA of embodiment 3, wherein the plurality of non-target immune cells comprises greater than 3 different cell types.

E6. The mRNA of embodiment 3, wherein the plurality of non-target immune cells comprises greater than 4 different cell types.

E7. The mRNA of any one of claim 1 or 2, wherein the target immune cell and the plurality of non-target immune cell are of different immune cell states.

E8. The mRNA of embodiment 7, wherein the plurality of non-target immune cells comprises between 2 and 5 different immune cell states.

E9. The mRNA of embodiment 7, wherein the plurality of non-target immune cells comprises greater than 3 different cell states.

E10. The mRNA of embodiment 7, wherein the plurality of non-target immune cells comprises greater than 4 different cell states.

E11. The mRNA of any one of embodiments 1-10, wherein the target immune cell and the plurality of non-target immune cells are of different immune cell lineages.

E12. The mRNA of any one of embodiments 1-10, wherein the target immune cell and the plurality of non-target immune cells are of different immune cell subpopulations.

E13. The mRNA of any one of embodiments 1-10, wherein the target immune cell and the plurality of non-target immune cells are of different activation states.

E14. The mRNA of any one of embodiments 1-10, wherein the target immune cell and the plurality of non-target immune cells are of different transformation states.

E15. The mRNA of any one of embodiments 1-14, wherein the target immune cell is a T cell.

E16. The mRNA of embodiment 15, wherein the miR is selected from the group consisting of: miR-146, miR-23a, miR-142, miR-150, miR-21, and a combination thereof.

E17. The mRNA of any one of embodiments 1-14, wherein the target immune cell is a dendritic cell (DC).

E18. The mRNA of embodiment 17, wherein the miR is selected from the group consisting of: miR-142, miR-223, and a combination miR-142 and miR-223.

E19. The mRNA of any one of embodiments 1-14, wherein the target immune cell is a neutrophil.

E20. The mRNA of embodiment 19, wherein the miR is selected from the group consisting of: miR-143, miR-23a, miR-142, miR-150, and a combination thereof.

E21. The mRNA of any one of embodiments 1-14, wherein the target immune cell is a natural killer (NK) cell.

E22. The mRNA of embodiment 21, wherein the miR is selected from the group consisting of: miR-146, miR-23a, miR-142, miR-223, and a combination thereof.

E23. The mRNA of any one of embodiments 1-14, wherein the target immune cell is a monocyte.

E24. The mRNA of embodiment 23, wherein the miR is selected from the group consisting of: miR-23a, miR-142, miR-223, and a combination thereof.

E25. The mRNA of any one of embodiments 1-14, wherein the target immune cell is a macrophage.

E26. The mRNA of embodiment 25, wherein the miR is selected from the group consisting of: miR-23a, miR-142, miR-223, and a combination thereof.

E27. The mRNA of any one of embodiments 1-26, wherein the plurality of non-target immune cells comprises at least two cell types selected from the group consisting of: a T cell, a DC, a neutrophil, an NK cell, a monocyte, and a macrophage.

E28. The mRNA embodiment 12, wherein the target immune cell is a regulatory T cell and the miR is differentially expressed in regulatory T cells relative to naïve T cells and effector T cells.

E29. The mRNA of embodiment 28, wherein the miR is miR-146a-5p.

E30. The mRNA embodiment 13, wherein the target immune cell is an activated T cell and the miR is differentially expressed in the activated T cell relative to an unstimulated T cell.

E31. The mRNA of embodiment 31, wherein the miR is miR-155a-5p or miR-132-3p.

E32. The mRNA embodiment 14, wherein the target immune cell is a normal immune cell and the miR is differentially expressed in the normal immune cell relative to a cancerous immune cell.

E33. The mRNA of embodiment 32, wherein the normal immune cell is a bone marrow cell, a B cell, a T cell, a monocyte, a macrophage, a dendritic cell or any combination thereof.

E34. The mRNA of embodiment 33, wherein the cancerous immune cell is an AML cell.

E35. The mRNA of embodiment 34, wherein the miR is miR-150-5p, miR-146b-5p, miR-4286, miR-579-3b, miR-4516, miR-146a-5p, miR-664b-3p, miR-342-3p, miR-342-5p, miR-1915-3p, or miR-26b-5p.

E36. The mRNA embodiment 14, wherein the target immune cell is a cancerous immune cell and the miR is differentially expressed in the cancerous immune cell relative to a normal immune cell.

E37. The mRNA of embodiment 36, wherein the normal immune cell is a bone marrow cell, a B cell, a T cell, a monocyte, a macrophage, a dendritic cell or any combination thereof.

E38. The mRNA of embodiment 37, wherein the cancerous immune cell is an AML cell.

E39. The mRNA of embodiment 38, wherein the miR is miR-18a-5p, miR-1246, or miR-126-3p.

E40. The mRNA of any one of the preceding claims, wherein the miR is abundantly expressed in the target immune cell relative to the plurality of non-target immune cells.

E41. The mRNA of any one of embodiments 1-40, wherein the polypeptide of interest is a secreted protein.

E42. The mRNA of any one of embodiments 1-40, wherein the polypeptide of interest is an intracellular protein.

E43. The mRNA of any one of embodiments 1-40, wherein the polypeptide of interest is a transmembrane or membrane-bound protein.

E44. The mRNA of any one of embodiments 1-40, wherein the polypeptide of interest is a cytotoxic polypeptide.

E45. The mRNA of any one of embodiments 1-44, comprising 2-5 miR-binding sites.

E46. The mRNA of any one of embodiments 1-44, comprising two miR-binding sites, three miR-binding sites, or 4 miR-binding sites.

E47. The mRNA of any one of the preceding claims, wherein the miR-binding site(s) is located in the 3′ UTR.

E48. The mRNA of any one of embodiments 1-46, wherein the miR-binding site(s) is located in the 5′ UTR.

E49. The mRNA of any one of the preceding claims, wherein the mRNA is fully modified with chemically-modified uridines.

E50. The mRNA of embodiment 49, wherein the chemically-modified uridines comprise N1-methyl-pseudouridine (m1ψ), 5-methoxy-uridine (mo5U), 5-methyl-cytidine (m5C), pseudouridine (ψ), α-thio-guanosine, or α-thio-adenosine, or a combination thereof.

E51. The mRNA of embodiment 50, wherein the chemically-modified uridines are N1-methylpseudouridines (m1ψ).

E52. A lipid nanoparticle (LNP) comprising the mRNA of any one of the preceding claims.

E53. A pharmaceutical composition comprising the mRNA of any one of embodiments 1-51, or the LNP of embodiment 52, and a pharmaceutically acceptable carrier.

E54. The pharmaceutical composition of embodiment 53, for use in treating or delaying progression of a disease or disorder in a subject, wherein the treatment comprises administration of the pharmaceutical composition.

E55. Use of the pharmaceutical composition of embodiment 53, in the manufacture of a medicament for treating or delaying progression of a disease or disorder in a subject, wherein the medicament comprises the pharmaceutical composition, and wherein the treatment comprises administration of the medicament.

E56. A kit comprising a container comprising the pharmaceutical composition embodiment 53, and a package insert comprising instructions for administration of the pharmaceutical composition for treating or delaying progression of a disease or disorder in a subject.

E57. A method of inducing selective degradation of an mRNA in a target immune cell relative to a plurality of non-target immune cells, comprising contacting a target immune cell with an mRNA of any one of embodiments 1-51, or an LNP of embodiment 52, optionally with a pharmaceutically acceptable carrier, such that the mRNA is selectively degraded in the target immune cell relative to the plurality of non-target immune cells.

E58. An immune cell delivery LNP comprising:

(i) an ionizable lipid;

(ii) a sterol or other structural lipid;

(iii) an mRNA molecule of any one of embodiments 1-51;

(iii) optionally, a non-cationic helper lipid or phospholipid; and

(iv) optionally, a PEG-lipid;

wherein one or more of (i) the ionizable lipid or (ii) the sterol or other structural lipid comprises an immune cell delivery potentiating lipid in an amount effective to enhance delivery of the LNP to a target immune cell and a plurality of non-target immune cells.

E59. The immune cell delivery LNP of embodiment 58, which comprises a phytosterol or a combination of a phytosterol and cholesterol.

E60. The immune cell delivery LNP of embodiment 59, wherein the phytosterol is selected from the group consisting of β-sitosterol, stigmasterol, β-sitostanol, campesterol, brassicasterol, and combinations thereof.

E61. The immune cell delivery LNP of embodiment 59, wherein the phytosterol comprises a sitosterol or a salt or an ester thereof.

E62. The immune cell delivery LNP of embodiment 59, wherein the phytosterol comprises a stigmasterol or a salt or an ester thereof.

E63. The immune cell delivery LNP of embodiment 59, wherein the phytosterol is beta-sitosterol

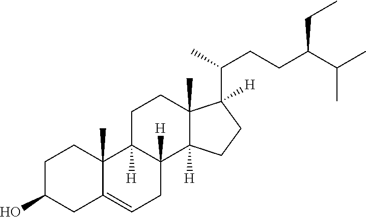

or a salt or an ester thereof.

E64. The immune cell delivery lipid LNP of embodiment 59, wherein the phytosterol or a salt or ester thereof is selected from the group consisting of p-sitosterol, p-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof.

E65. The immune cell delivery LNP of embodiment 64, wherein the phytosterol is β-sitosterol.

E66. The immune cell delivery LNP of embodiment 64, wherein the phytosterol is β-sitostanol.

E67. The immune cell delivery LNP of embodiment 64, wherein the phytosterol is campesterol.

E68. The immune cell delivery LNP of embodiment 64, wherein the phytosterol is brassicasterol.

E69. The immune cell delivery LNP of any one of embodiments 58-68, wherein the ionizable lipid comprises a compound of any of Formulae (I I), (I IA), (IIB), (III), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8).

E70. The immune cell delivery LNP of any one of embodiments 58-68, wherein the ionizable lipid comprises a compound selected from the group consisting of Compound X, Compound Y, Compound I-48, Compound I-50, Compound I-109, Compound I-111, Compound 1-113, Compound I-181, Compound I-182, Compound I-244, Compound I-292, Compound I-301, Compound I-309, Compound I-317, Compound I-321, Compound I-322, Compound I-326, Compound I-328, Compound I-330. Compound I-331, Compound I-332, Compound I-347, Compound I-348, Compound I-349, Compound I-350, Compound I-352 and Compound I-M.

E71. The immune cell delivery LNP of any one of embodiments 58-68, wherein the ionizable lipid comprises a compound selected from the group consisting of Compound X, Compound Y, Compound I-321. Compound I-292, Compound I-326, Compound I-182, Compound I-301, Compound I-48, Compound I-50, Compound I-328, Compound I-330, Compound I-109, Compound I-111 and Compound I-181.

E72. The immune cell delivery LNP of any one of embodiments 58-71, wherein the LNP comprises a phospholipid, and wherein the phospholipid comprises a compound selected from the group consisting of DSPC, DMPE, and Compound H-409.

E73. The immune cell delivery LNP of any one of embodiments 58-72, wherein the LNP comprises a PEG-lipid.

E74. The immune cell delivery LNP of embodiment 73, wherein the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

E75. The immune cell delivery LNP of embodiment 73, wherein the PEG lipid comprises a compound selected from the group consisting of Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25.

E76. The immune cell delivery LNP of embodiment 75, wherein the PEG lipid comprises a compound selected from the group consisting of Compound P-428, Compound PL-16, Compound PL-17, Compound PL-18, Compound PL-19, Compound PL-1, and Compound PL-2.

E77. The immune cell delivery LNP of any one of embodiments 58-76, which comprises about 30 mol % to about 60 mol % ionizable lipid, about 0 mol % to about 30 mol % non-cationic helper lipid or phospholipid, about 18.5 mol % to about 48.5 mol % sterol or other structural lipid, and about 0 mol % to about 10 mol % PEG lipid.

E78. The immune cell delivery LNP of any one of embodiments 58-76, which comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid.

E79. The immune cell delivery LNP of any one of embodiments 58-76, which comprises about 50 mol % ionizable lipid, about 10 mol % non-cationic helper lipid or phospholipid, about 38.5 mol % sterol or other structural lipid, and about 1.5 mol % PEG lipid.

E80. The immune cell delivery LNP of any one of embodiments 77-79, wherein the mol % sterol or other structural lipid is 18.5% phytosterol and the total mol % structural lipid is 38.5%.

E81. The immune cell delivery LNP of any one of embodiments 77-79, wherein the mol % sterol or other structural lipid is 28.5% phytosterol and the total mol % structural lipid is 38.5%.

E82. The immune cell delivery LNP of any one of embodiments 58-76, which comprises:

(i) about 50 mol % ionizable lipid, wherein the ionizable lipid is a compound selected from the group consisting of Compound I-301, Compound I-321, and Compound I-326;

(ii) about 10 mol % phospholipid, wherein the phospholipid is DSPC;

(iii) about 38.5 mol % structural lipid, wherein the structural lipid is selected from β-sitosterol and cholesterol; and

(iv) about 1.5 mol % PEG lipid, wherein the PEG lipid is Compound P-428.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

### Example 1: Identification of Differentially Expressed miRs in Human Immune Cell Subsets

Nanostring human miR panel (NS_H_MIR_V3A, Nanostring, Washington) was employed to determine expression levels of miRs in human immune cells. In this experiment, immune cells were isolated from PBMCs obtained from a healthy human donor using fluorescence activated cell sorting (FACS). Using specific antibodies (anti human CD19− APC; anti human CD3-BV605; anti human CD11b-PE and anti-human CD14-PerCPCy5.50), cells were isolated using the following gating strategy:

T cells: CD3+CD19-CD11b-CD14-;

B cells: CD3-CD19+CD11b-CD14-;

Monocytes: CD3-CD19+CD11b+CD14high

Macrophages: CD3-CD19+CD11b+CD14low

Total mRNAs were extracted from each cell population using Promega Maxwell instrument for RNA extraction using the miRNA tissue kit (#AS1460). One hundred nanograms of RNA from each cell population was loaded for analysis using the miR panel. Expression for a given miR was determined as percentage of total detectable miRs in a given population.

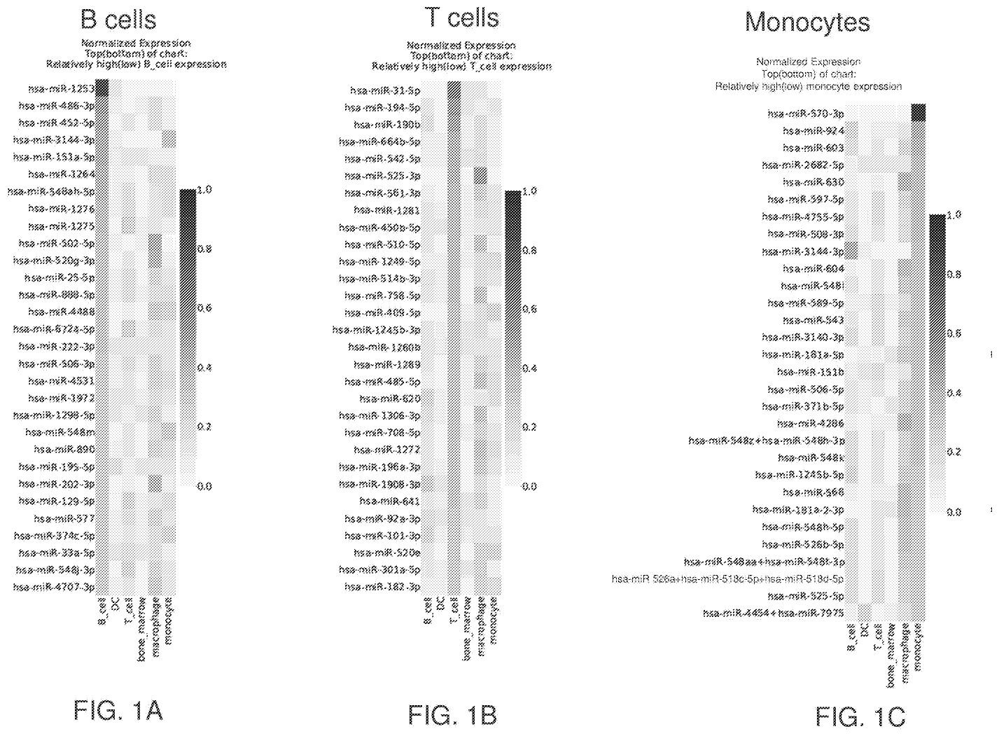

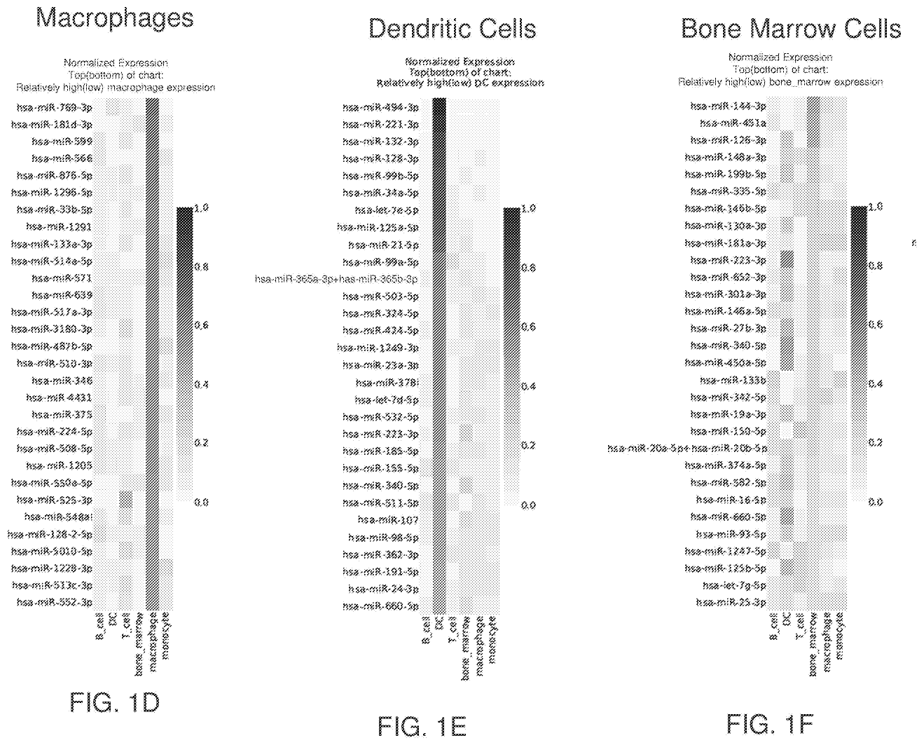

Certain miRs were found to be differentially expressed in various immune cell populations as shown by the heat maps in FIGS. 1A-1F. The heat maps provides a matrix depicting miR expression level for thirty miRs across six different immune cell types, including B cells, bone marrow cells, T cells, DCs, macrophages, and monocytes. The level of expression of a given miR is represented in a row of the heat map and each cell type is represented by a column of the heat map. For each row, the level of expression of a given miR is normalized such that the total level of expression among all six cell types yields a total intensity equal to 1. The miRs are then sorted from those that are most differentially expressed to those that are least differentially expressed. In a heat map sorted based upon a given cell type (e.g., the target cell type), the value of normalized differential expression for the target cell type is higher in a given row compared to any row below. For the plurality of all six immune cell types, the normalized expression is more distributed across all cell types for a given row compared to any row above.

For the six cell types, the normalized expression cutoff values used to identify the top thirty differentially expressed miRs in a given cell type are shown in Table 26.

TABLE 26
Normalized expression cutoff for top 30 candidates by cell type:

| Immune cell type | Cutoff Value |
|---|---|
| B-cell | 0.284 |
| DC | 0.461 |
| T-cell | 0.315 |
| Bone marrow | 0.199 |
| Macrophage | 0.521 |
| Monocyte | 0.314 |

The miRs most differentially expressed in B cells compared to other immune cell types (e.g., healthy bone marrow, T cells, DCs, macrophages, and monocytes) are shown by the heat map in FIG. 1A and listed in Table 1. The miRs most differentially expressed in T cells compared to other immune cell types (e.g., healthy bone marrow. B cells, DCs, macrophages, and monocytes) are shown by the heat map in FIG. 1B and listed in Table 2. The miRs most differentially expressed in monocytes compared to other immune cell types (e.g., healthy bone marrow, T cells, B cells, DCs, and macrophages) are shown by the heat map in FIG. 1C and listed in Table 4. The miRs most differentially expressed in macrophages compared to other immune cell types (e.g., healthy bone marrow, B cells, T cells, DCs, and monocytes) are shown by the heat map in FIG. 1D and listed in Table 5. The miRs most differentially expressed in DCs compared to other immune cell types (e.g., healthy bone marrow, B cells, T cells, macrophages, and monocytes) are shown by the heat map in FIG. 1E and listed in Table 3. The miRs most differentially expressed in bone marrow cells compared to other immune cell types (e.g., B cells, T cells, DCs, macrophages, and monocytes) are shown by the heat map in FIG. 1F and listed in Table 6.

### Example 2: Identification of Abundantly Expressed miRs in Immune Cell Subsets in Humans and Rodents

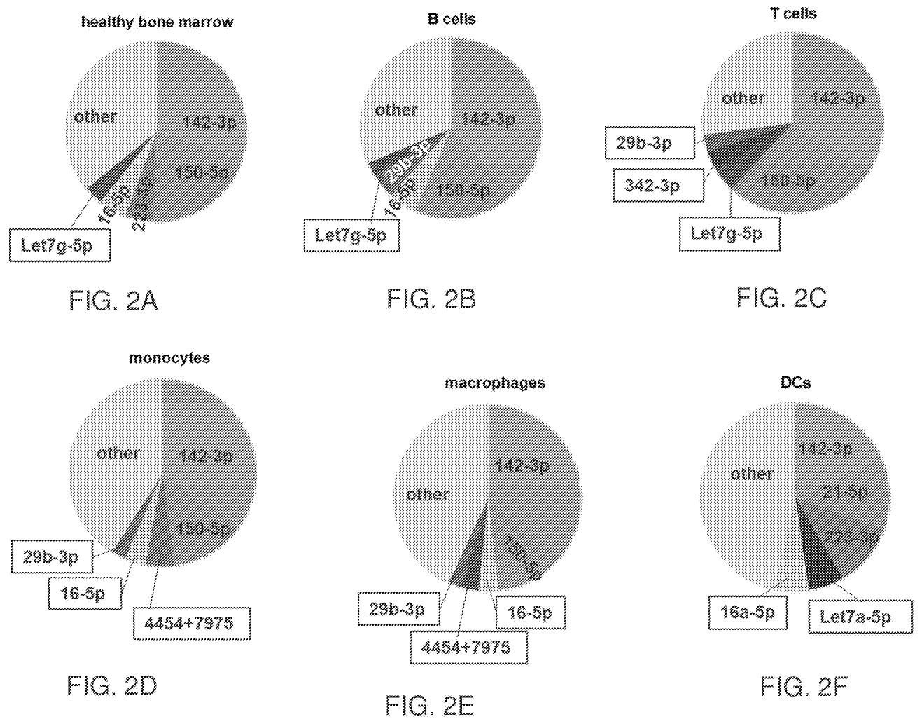

The relative abundance of each miR as a fraction of total analyzed miRs are summarized in the pie charts shown in FIGS. 2A-2F. For each cell type, top 5 most abundant miRs are shown. As shown in these charts, mir142-3p represented the largest fraction of miRs expressed in healthy bone marrow obtained from a bone marrow biopsy from one donor (FIG. 2A), B cells (FIG. 2B), T cells (FIG. 2C), monocytes (FIG. 2D), macrophages (FIG. 2E), and dendritic cells “DCs” (FIG. 2F). miR-150 represented the second most abundantly expressed miR in healthy bone marrow, B cells, T cells, monocytes and macrophages. In contrast, miR-150-5p was not found to be an abundant miR expressed by dendritic cells.

Additionally, miR expression levels were evaluated in immune cell subsets isolated from a mouse model. To do so, Nanostring was employed to determine expression levels of miRs in mouse immune cells. In this experiment, immune cells were isolated from mouse splenocytes by labeling the cells with antibodies specific to cell-surface marks and sorting the cells by FACS. Cells were sorted using the following marker specific antibodies: anti-mouse-CD19-APC; anti-mouse-CD3-FITC; anti-mouse-CD11c-PE and anti-mouse-CD11b-PerCp-Cy5.5, anti-mouse-CD68-BV421. Mouse DCs were purchased (Cell Biologics #57-6200).

Total mRNAs were extracted from each cell population using Promega Maxwell instrument for RNA extraction using the miRNA tissue kit (#AS1460). One hundred nanograms of RNA from each cell population was loaded for analysis using the miR panel. Expression for a given miR was determined as percentage of total detectable miRs in a given population.

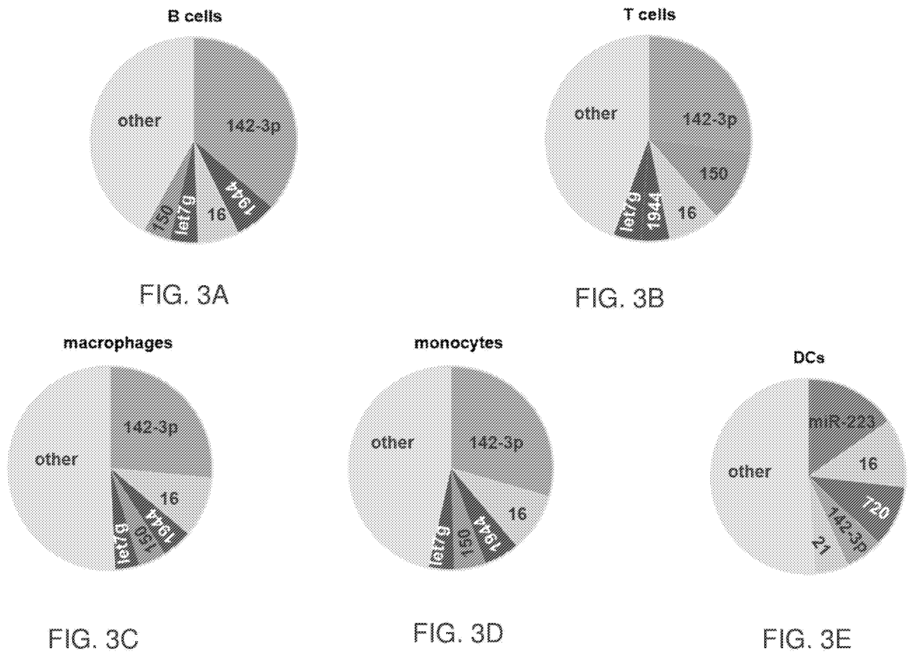

The relative abundance of each miR as a fraction of total analyzed miRs are summarized in the pie charts shown in FIGS. 3A-3E For each cell type, the top five most abundant miRs are shown. As shown in these charts, mir142-3p represented the largest fraction of miRs expressed in mouse B cells (FIG. 3A), T cells (FIG. 3B), macrophages (FIG. 3C), and monocytes (FIG. 3D). Other abundant miRs expressed in mouse immune cells included miR-150, miR-1944, miR-16, and miRlet7. The most abundant miRs expressed by mouse dendritic cells was different from the other immune cells evaluated. While, miR-142-3p was identified as an abundant miR in mouse dendritic cells, it represented a smaller fraction of total miRs compared to the other immune cell subsets (FIG. 3E). Additionally, miR-223 was identified as the most abundant miR expressed in mouse dendritic cells, but was not identified as an abundant miR in the other immune cell subsets evaluated.

These pie charts show that similar to human immune cells, mouse immune cells also highly express miR142-3p. However, the relative abundance of miR-150-5p is variable between the different mouse immune cell types (e.g., second highest in T cells, fifth highest in B cells, and not a top five highly expressed miR in dendritic cells), whereas miR-150-5p is consistently the second most abundantly expressed miR after miR-142-3p in all human immune cell subsets evaluated except dendritic cells (FIGS. 3A-3F).

### Example 3: Expression of mRNA Encoding miR Binding Sites in Immune Cells In Vitro

The effect of mRNA comprising miR binding sites corresponding to differentially expressed miRs was evaluated on mRNA expression in immune cells in vitro. PBMCs were isolated and were treated with mOX40L mRNA constructs engineered with or without miR binding sites. The cells were analyzed by flow cytometry to determine OX40L expression for a given immune cell type. The immune cell subsets were distinguished using antibodies specific for cell surface markers (anti-human-CD20-AF700; anti-human-CD3-BV421; anti-human-CD56-BV605; anti-human-CD11b-PerCP-Cy5.5; anti-human-CD14-PE-Cy7; anti-human-CD16-FITC; anti-human-CD11c-BV711) cells were characterized using the following gating strategy:

T cells: CD20− CD3+;

NK T cells: CD20− CD3+CD56+;

B cells: CD3− CD20+CD11b−;

Granulocytes: CD3− CD20− CD11b+;

Neutrophils: CD3− CD20− CD11b−;

Classical Monocytes: CD3− CD20− CD14+CD16−

Non-classical Monocytes: CD3− CD20− CD14− CD16+

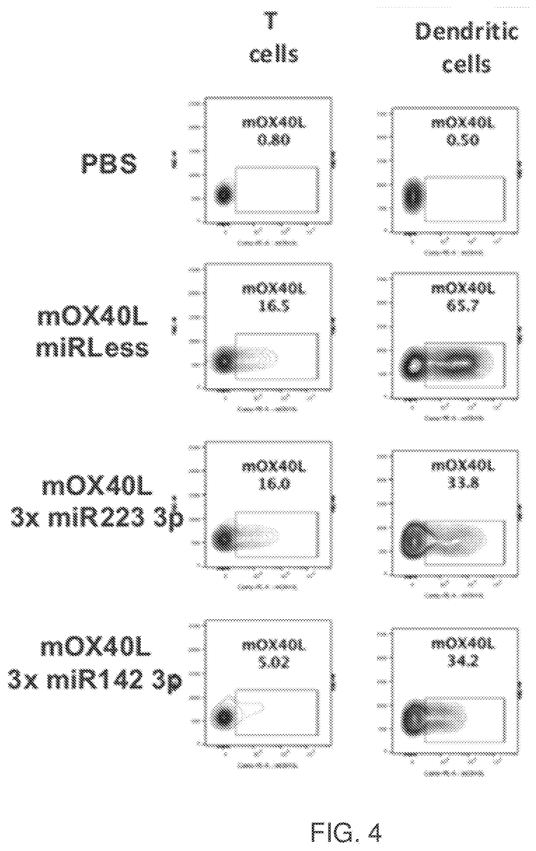

The effect of mRNA encoding OX40L comprising three miR-223-3p binding sites in the 3′UTR or three miR-142-3p binding sites in the 3′UTR was evaluated on OX40L expression as shown in FIG. 4. In T cells it was found that inclusion of miR-142-3p binding sites resulted in a substantial decrease in OX40L expression relative to mRNA lacking miR binding sites. This was expected given that miR-142-3p is abundantly expressed in T cells. Inclusion of miR-142-3p binding sites also resulted in decreased mRNA expression in other cell subsets evaluated, including dendritic cells, neutrophils, granulocytes, NK cell subsets, and monocyte cell subsets (data not shown). Treatment with mRNA encoding miR-223-3p binding sites resulted in an approximately 50% reduction in mRNA expression in dendritic cells as shown in FIG. 4. This was anticipated given that miR-223-3p was found to be highly expressed in dendritic cells. Additionally, inclusion of a miR-223-3p binding site resulted in reduction in mRNA expression in other immune cell subsets, including NK cell subsets and monocyte cell subsets (data not shown). However, inclusion of miR-223-3p binding sites had limited effect on expression in T cells (FIG. 4). Thus, differential expression of mRNA in dendritic cells relative to T cells was achieved using mRNA encoding a miR-223-3p binding site.

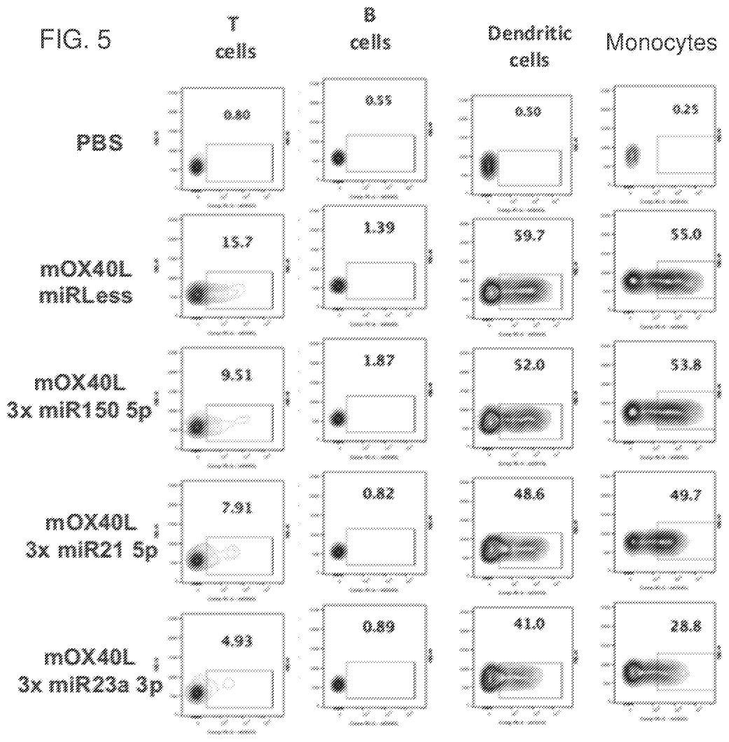

The effect of mRNA encoding OX40L comprising three miR-150-5p binding sites in the 3′UTR, three miR-21-5p binding sites in the 3′UTR or three miR-23a-3p binding site in the 3′UTR was evaluated on OX40L expression as shown in FIG. 5. In T cells it was found that inclusion of miR-150-5p binding sites resulted in a substantial decrease in OX40L expression relative to mRNA lacking a miR binding site. Additionally, inclusion of miR-150-5p binding sites resulted in decreased mRNA expression in neutrophils (data not shown). However, inclusion of miR-150-5p binding sites had no effect on mRNA expression in dendritic cells and monocytes as shown in FIG. 5, and neither in NK cell subsets (data not shown). In T cells it was found that inclusion of miR-21-5p binding sites resulted in a substantial decrease in OX40L expression relative to mRNA lacking a miR binding site, but had limited effect on expression in other immune cell subsets evaluated, including dendritic cells (FIG. 5). Finally, in T cells and monocytes it was found that inclusion of miR-23a-3p binding sites resulted in a substantial decrease in OX40L expression relative to mRNA lacking a miR binding site (FIG. 5). Additionally, inclusion of miR-23a-3p binding sites resulted in decreased mRNA expression in neutrophils and NK cell subsets (data not shown). However, inclusion of miR-23a-3p binding sites resulted in only a small reduction in mRNA expression in dendritic cells as shown in FIG. 5.

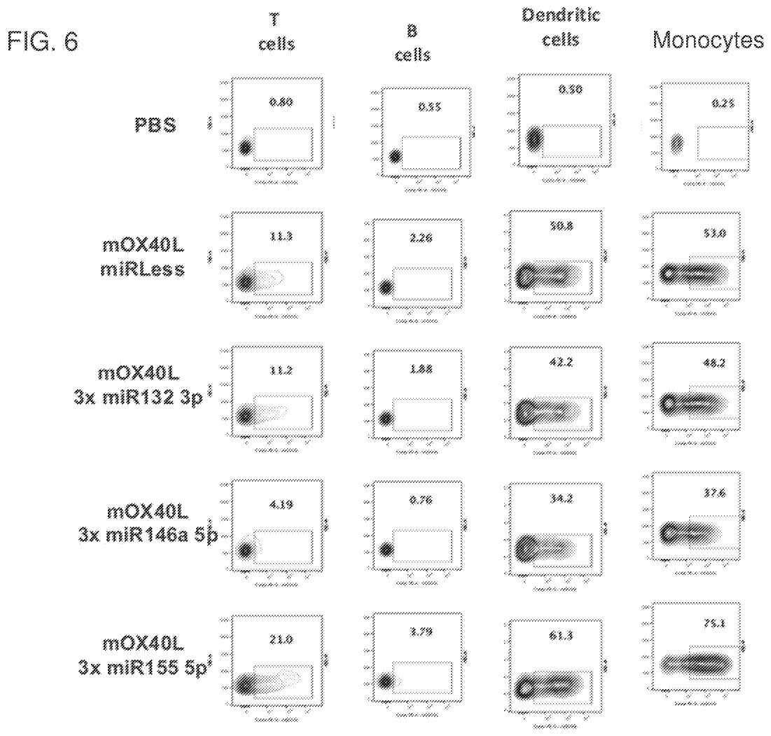

The effect of mRNA encoding OX40L comprising a three miR-132-3p binding sites in the 3′UTR, three miR-146-5p binding sites in the 3′UTR, or three miR-155-5p binding site in the 3′UTR was evaluated on OX40L expression as shown in FIG. 6. Inclusion of miR-132-3p binding sites had limited effect on OX40L expression relative to mRNA lacking miR binding sites in any immune cell subset evaluated, including T cells, NK cells, dendritic cells, neutrophils, or monocytes. However, inclusion of miR-146a-5p binding sites resulted in substantially decreased mRNA expression in T cells as shown in FIG. 6. A reduction in mRNA expression was also seen in NK cells (data not shown). However, inclusion of miR-146a-5p binding sites had limited effected on mRNA expression in dendritic cells and resulted in only a small reduction in mRNA expression in monocytes (FIG. 6). Inclusion of miR-155-5p binding sites did not result in reduced mRNA expression in any immune cell subset evaluated.

### Example 4: mOX40L Encoding mRNA Having 3′ UTR miR Binding Sites Decreased mOX40L Expression in Immune Cells In Vivo

To determine if miR binding sites could be used to suppress expression from mRNA in healthy immune cells in vivo, Sprague Dawley rats were intravenously injected with 0.3 mg/kg of mOX40L mRNA constructs engineered with or without miR binding sites. mRNA was encapsulated in lipid nanoparticles comprising compound X/DSPC/cholesterol/beta-sitosterol/PEG DMG at a ratio of 50:10:10:28.5:1.5. Such lipid nanoparticles (LNPs), which contain beta-sitosterol as an immune cell delivery potentiating lipid, are described further in PCT Application No. PCT/US19/15913, filed Jan. 30, 2019, the entire contents of which is expressly incorporated herein by reference.

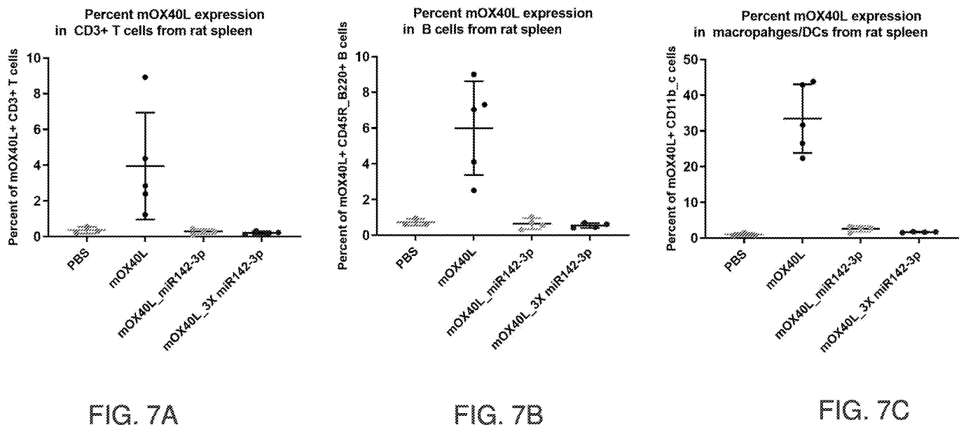

Twenty-four hours after injection, splenocytes were analyzed for mOX40L expression levels in T cells (FIG. 7A), B cells (FIG. 7B), or macrophages/dendritic (myeloid) cells (FIG. 7C). Administration of mOX40L mRNA constructs resulted in varying expression levels of mOX40L protein in T cells (mean 4%), B cells (mean 6%), and macrophages/DCs (mean 35%) (data not shown). mOX40L expression was reduced in rats that were administered mOX40L encoding mRNA comprising 3′UTRs having one miR-142-3p or three miR-142-3p binding sites as shown in FIGS. 7A-7C. This data showed that miR-142-3p binding sites on mRNA could be used to suppress expression of translated protein in healthy immune cells.

### Example 5: Expression of mRNA Encoding miR Binding Sites In Vivo

The effect of mRNA comprising miR binding sites corresponding to differentially expressed miRs was evaluated on mRNA expression in immune cells in vivo. The mRNA constructs that were evaluated encoded OX40L and comprised either zero, one or three miR binding sites in the 3′UTR. According to miR expression analysis described in Example 1, miR-21-5p and miR-23a-3p were found to be differentially expressed in dendritic cells. Thus, mRNA encoding one binding site complimentary to miR-21-5p and miR-23a-3p were evaluated. Additionally, mRNA encoding three binding site complimentary to miR-21-5p and miR-23a-3p were also evaluated. The mRNA constructs were modified by substitution of uracil with N1-methylpseudouracil. The mRNA constructs were encapsulated in lipid nanoparticles comprising compound X/DSPC/cholesterol/beta-sitosterol/PEG DMG at a ratio of 50:10:10:28.5:1.5 and a single dose was administered intravenously at 0.5 mg/kg into Jh mice. The Jh mouse model comprises a deletion in the antibody heavy chain that results in a mouse model devoid of mature B lymphocytes.

Following 24 hours post-administration, mRNA expression was evaluated in mouse splenocytes. The spleens were harvested and digested to generate a suspension of cells. The cells were stained with antibodies against cell surface markers and cell-surface staining was assessed by flow cytometry to distinguish immune cell types. An anti-mouse CD16/32 Fc block (TruStain FcX. Biolegend) as well as a viability dye (Zombie Aqua, Biolegend) were used prior to staining to inhibit non-specific antibody binding and to exclude dead cells from analysis. Expression of OX40L was evaluated by staining with an anti-mouse OX40L-PE antibody. The antibodies used for identification of cell markers included: anti-mouse-CD3-FITC, anti-mouse-CD19-AF700, anti-mouse-CD4-APC-Cy7, anti-mouse-CD8-APC, anti-mouse-CD11c-BV711, and anti-mouse CD11b-PerCP-Cy5.5. Immune cell subsets were identified by the following gating strategy:

CD8 T cells: CD3+CD19− CD4− CD8+

CD4 T cells: CD3+CD19− CD4+CD8−

B cells: CD3− CD19+

Macrophages: CD11b+CD11c−

Dendritic cells: CD11b− CD11c+

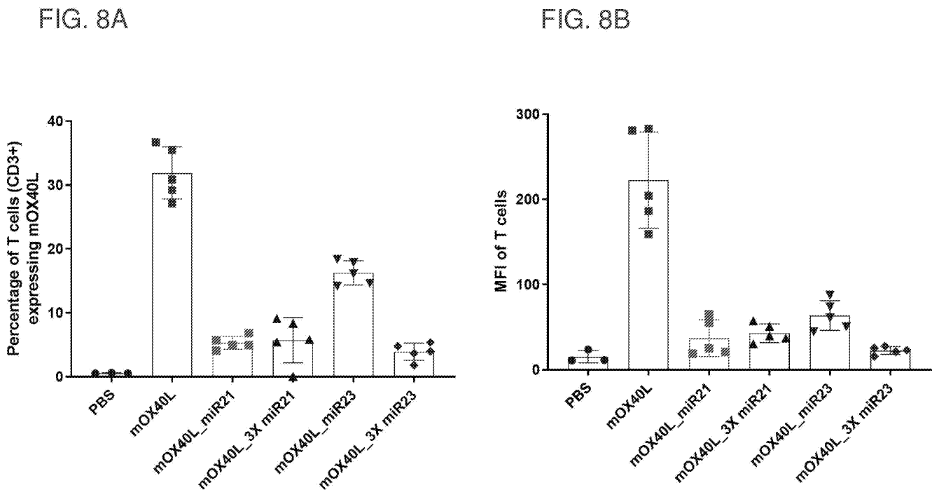

Expression of mRNA constructs in CD3+ T cells was evaluated. Shown in FIG. 8A is the percentage of CD3+ T cells that were positive for OX40L expression. Shown in FIG. 8B is the average level of cell-surface staining for OX40L on CD3+ T cells. Approximately 30% of CD3+ T cells expressed OX40L for mRNA incorporating no miR binding site, while inclusion of one or three miR-21-5p or miR-23a-3p binding sites resulted in a substantial decrease in the proportion of CD3 T cells expressing OX40L and the abundance of OX40L expressed at the cell surface.

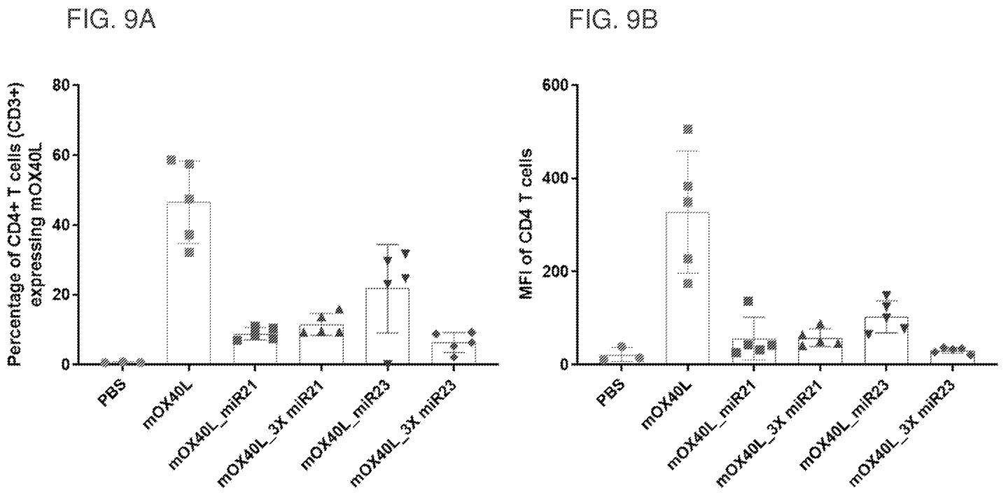

Expression of mRNA constructs in CD3+ T cells that were also CD4+ was evaluated. Shown in FIG. 9A is the percentage of CD4 T cells that were positive for OX40L expression. Shown in FIG. 9B is the average level of cell-surface staining for OX40L on CD4 T cells. Approximately 45% of CD4 T cells expressed OX40L for mRNA incorporating no miR binding site. Inclusion of one or three miR-21-5p or miR-23a-3p binding sites resulted in a substantial decrease in the proportion of CD4 T cells expressing OX40L and the abundance of OX40L expressed at the cell surface.

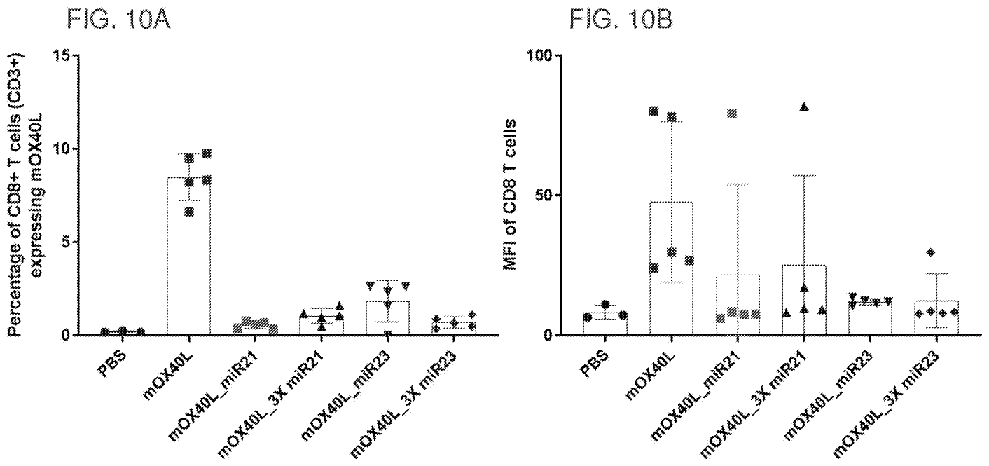

Expression of mRNA constructs in CD3+ T cells that were also CD8+ was evaluated. Shown in FIG. 10A is the percentage of CD8 T cells that were positive for OX40L expression. Shown in FIG. 10B is the average level of cell-surface staining for OX40L on CD8 T cells. Approximately 10% of CD8 T cells expressed OX40L for mRNA incorporating no miR binding site. Inclusion of one or three miR-21-5p or miR-23a-3p binding sites resulted in a substantial decrease in the proportion of CD8 T cells expressing OX40L and the abundance of OX40L expressed at the cell surface.

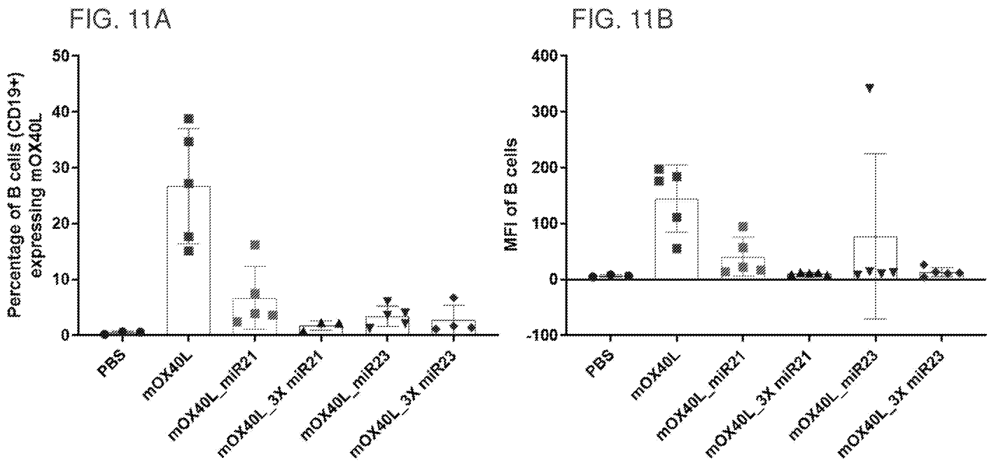

Expression of mRNA constructs in B cells was evaluated. Shown in FIG. 11A is the percentage of B cells that were positive for OX40L expression. Show in FIG. 11B is the average level of cell-surface staining for OX40L on B cells. Approximately 25% of B cells expressed OX40L for mRNA incorporating no miR binding site. Inclusion of one or three miR-21-5p or miR-23a-3p binding sites resulted in a substantial decrease in the proportion of B cells expressing OX40L and the abundance of OX40L expressed at the cell surface.

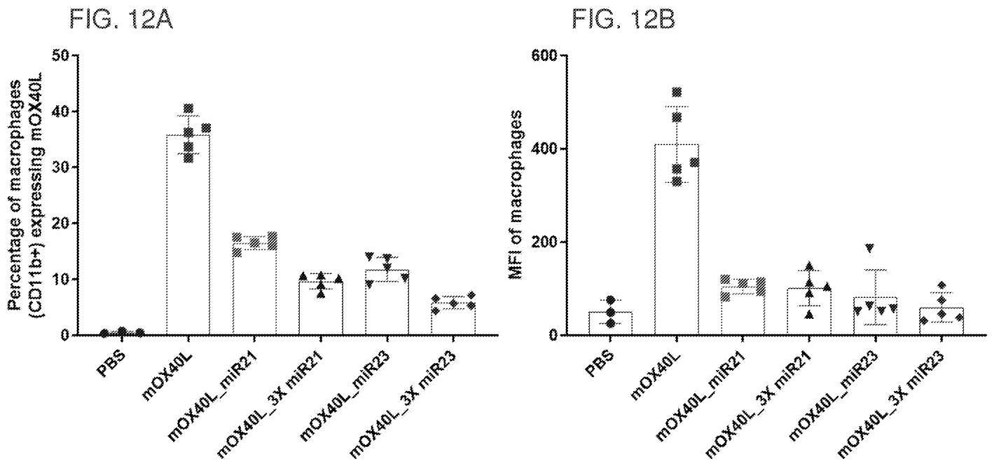

Expression of mRNA constructs in macrophages was evaluated. Shown in FIG. 12A is the percentage of macrophages that were positive for OX40L expression. Show in FIG. 12B is the average level of cell-surface staining for OX40L on macrophages. Approximately 35% of macrophages expressed OX40L for mRNA incorporating no miR binding site. Inclusion of one or three miR-21-5p or miR-23a-3p binding sites resulted in a substantial decrease in the proportion of macrophages expressing OX40L and the abundance of OX40L expressed at the cell surface. In particular, for either miR-21-5p or miR-23a-3p, if three binding sites were encoded rather than one, a more substantial decrease in the frequency of OX40L expression was seen. Thus, expression in macrophages can be modulated based upon the number of miR-21-5p or miR-23a-3p binding sites that are encoded into the mRNA.

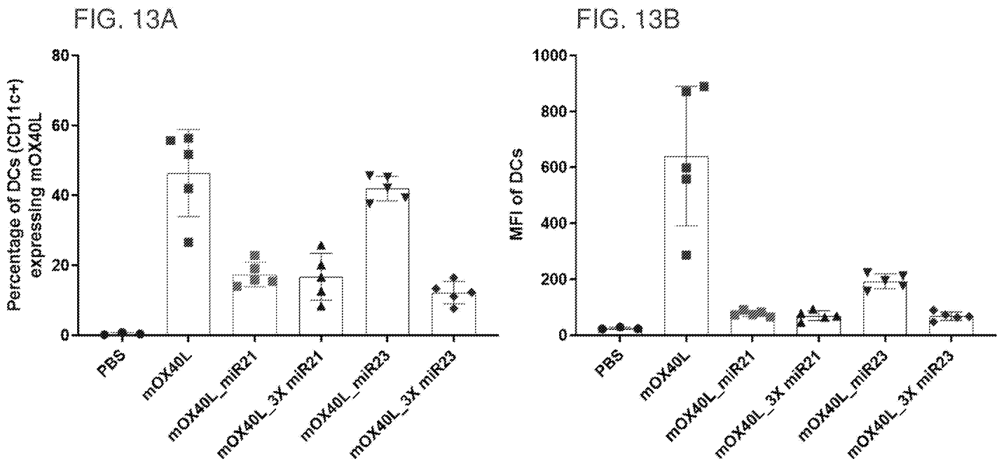

Expression of mRNA constructs in dendritic cells was evaluated. Shown in FIG. 13A is the percentage of dendritic cells that were positive for OX40L expression. Show in FIG. 13B is the average level of cell-surface staining for OX40L on dendritic cells. Approximately 45% of dendritic cells expressed OX40L for mRNA incorporating no miR binding site. In contrast, inclusion of one or three miR-21-5p binding sites resulted in a substantial decrease in the proportion of dendritic cells that expressed OX40L as well as the abundance of OX40L expressed at the cell surface. Inclusion of miR-23a-3p binding sites also resulted in a decrease in the proportion of dendritic cells expressing OX40L, however the decrease was more substantial if three miR-23a-3p binding sites were encoded in the mRNA rather than a single miR-23a-3p binding site

### Example 6: Identification of Highly Abundant miRs in Mouse Tumor Cell Lines

Nanostring human miR panel (NS_H_MIR_V3A, Nanostring, Washington) was employed to determine expression levels of miRs in mouse tumor cell lines. In this experiment, total mRNAs were extracted from each cell lines using Promega Maxwell instrument for RNA extraction using the miRNA tissue kit (#AS1460). One hundred nanograms of RNA from each cell population was loaded for analysis using the miR panel. Expression for a given miR was determined as percentage of total detectable miRs in a given population.

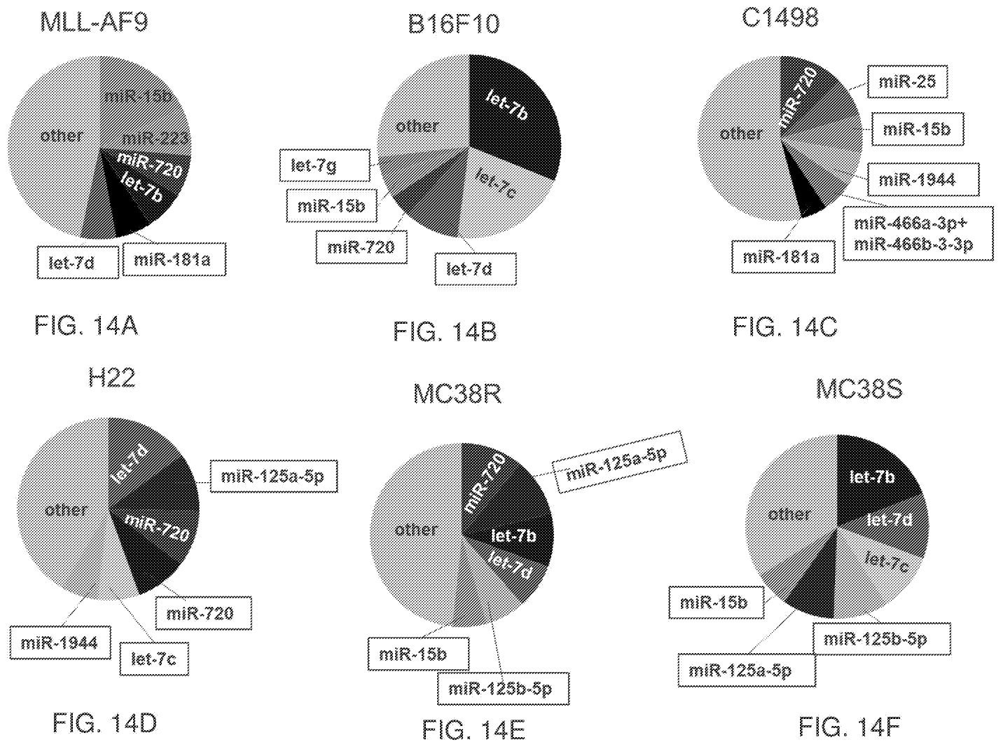

The relative abundance of each miR as a fraction of total analyzed miRs are summarized in the pie charts shown in FIGS. 14A-14F. For each tumor cell line, the top six most abundant miRs are shown. As shown in these charts, miR expression levels vary between various in vitro tumor cell lines tested: MLL-AF9 (FIG. 14A), B16F10 (FIG. 14B), C1498 (FIG. 14C), H22 (FIG. 14D), MC38R (FIG. 14E), and M38S (FIG. 14F). Nevertheless, certain miRs are detected as abundantly expressed in the tumor cell lines: let-7b (5/6 cell lines), let-7d (5/6 cell lines), miR-720 (5/6 cell lines), miR-15b (5/6 cell lines).

These pie charts thus show that miRs are differentially expressed in various tumor cell lines.

### Example 7: Immune Cells-Specific miR Expression Levels in Healthy Immune Cells Compared to AML Cell Lines

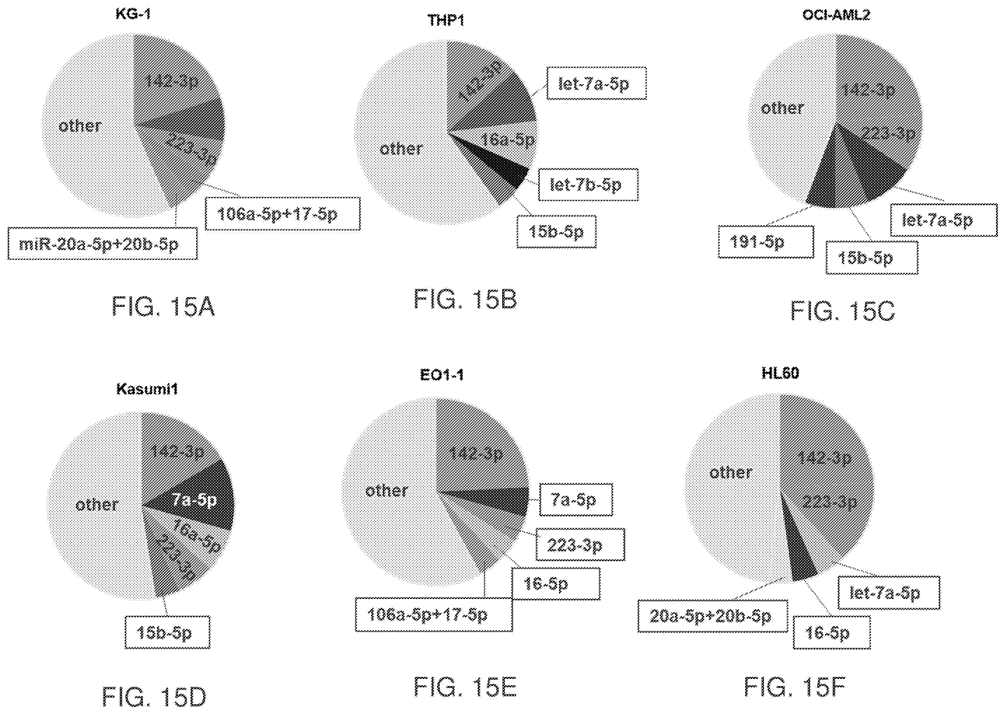

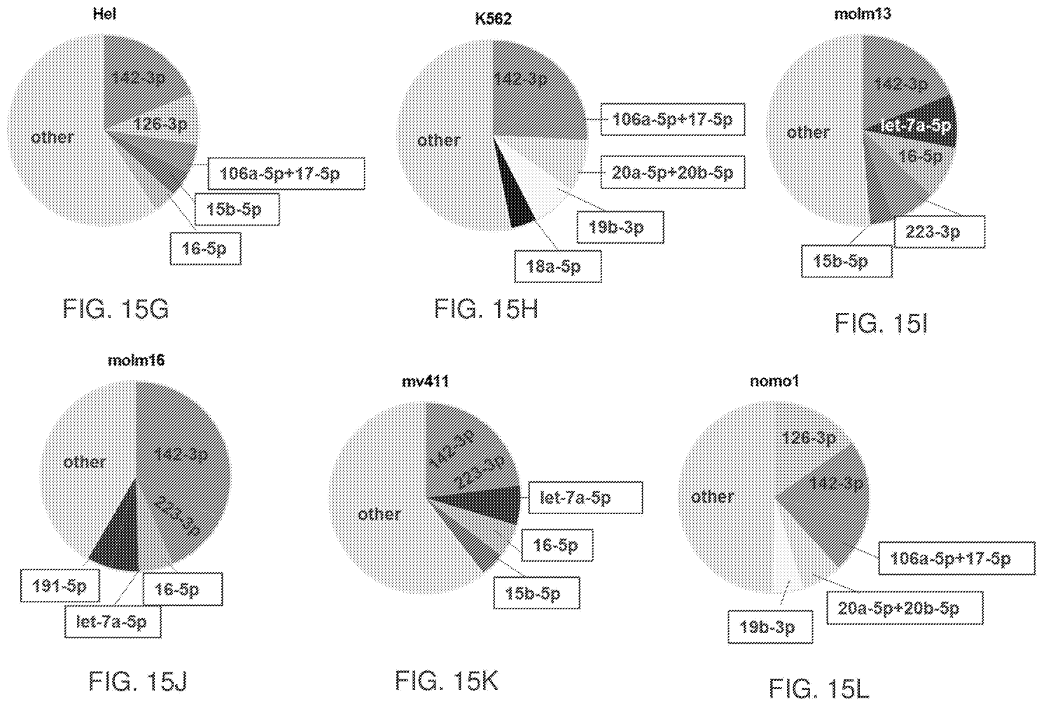

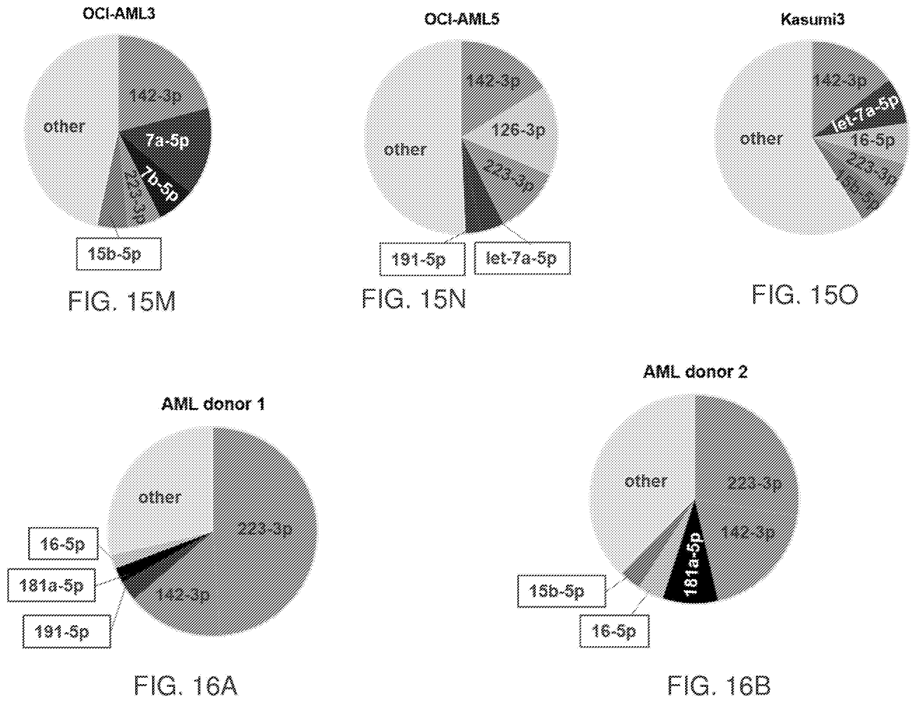

The most abundant miRs expressed in immortalized human AML cell lines was evaluated. To analyze differential miR expression, total RNAs were isolated from 15 immortalized AML cell lines. miR expression levels were measured as described in Example 1. Shown in FIGS. 15A-15O are pie charts representing the six miRs that were most abundantly expressed in each cell line, including KG-1 cells (FIG. 15A), THP1 cells (FIG. 15B), OCI-AML2 cells (FIG. 15C), Kasumil cells (FIG. 15D), EO1-1 cells (FIG. 15E), HL60 cells (FIG. 15F), Hel cells (FIG. 15G), K562 cells (FIG. 15H). molm13 cells (FIG. 15I), molm16 cells (FIG. 15J), mv411 cells (FIG. 15K), nomo1 cells (FIG. 15L), OCI-AML3 cells (FIG. 15M), OCI-AML5 cells (FIG. 15N), or Kasumi3 cells (FIG. 15O). These data highlight that similar to healthy immune cells, miR-142-3p is high abundant miR expressed by AML cells. Additionally, tumors cells were obtained from human patients with AML. miR abundance in AML cells obtained from human donors was evaluated. As shown in FIGS. 16A-16B, both miR-142-3p and miR-223-3p are abundantly expressed.

The miR expression levels (as percent of total detectable miRs) were compared to miR expression levels in healthy immune cells. Shown in Table 27 are the AML cells and immune cell populations for which miR abundance was evaluated.

TABLE 27

| Immortalized AML | Immune cell |
|---|---|
| cell lines | population |
| OCL AML2 | Bone marrow |
| OCL AML3 | B cells |
| OCL AML5 | T cells |
| KG-1 | Monocytes |
| Molm13 | Macrophages |
| HL60 | DCs |
| F-36P | |
| Kasumi-1 | |
| Kasumi-3 | |
| THP1 | |
| HEL | |
| K562 | |
| EO1-1 | |
| Mv4-11 | |
| Nomo1 | |

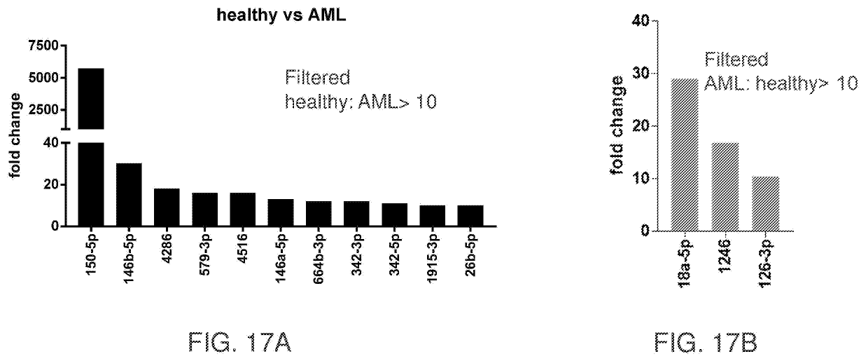

Nanostring nsolver package (Nanostring, Washington) was used to evaluate differentially expressed miRs by clustering all the healthy immune cells in one cluster (bone marrow, B cells, T cells, DCs, monocytes and macrophages) vs all the AML lines in the other cluster. Shown in FIGS. 17A-17B. are bar graphs identifying the miRs that were differentially expressed between healthy immune cells and AML cells. Shown in FIG. 17A are the miRs that were expressed at least 10-fold higher in the healthy immune cells cluster vs the AML lines cluster. While shown in FIG. 17B are miRs that were expressed at least 10-fold higher in AML cell cluster vs. healthy immune cells cluster. miR-150-5p expression levels decreased precipitously in AML cells, with a 5,000-fold decrease in expression compared to healthy immune cells. In contrast, the second largest decrease was approximately a 30-fold decrease in miR-146-5p expression.

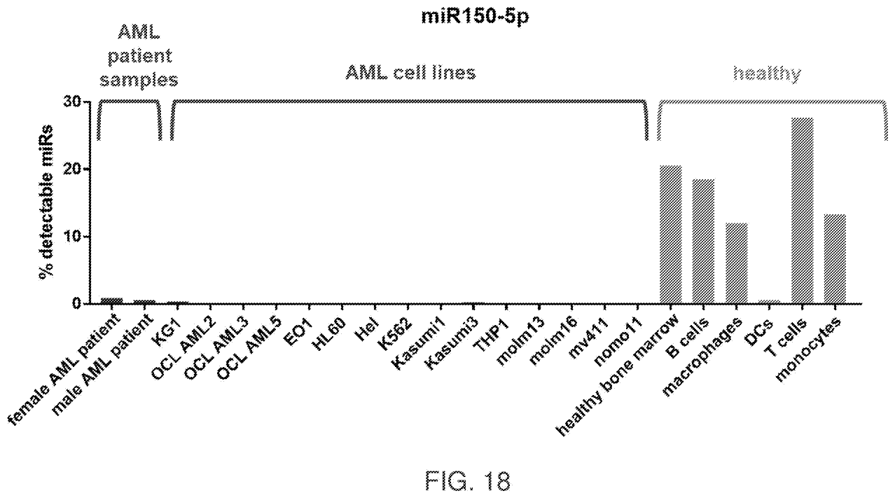

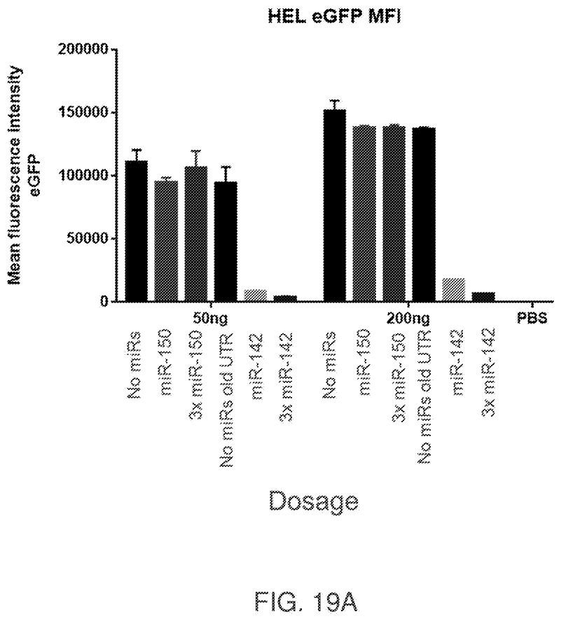

To determine if the decreased miR-150-5p observed in AML cell lines was representative of primary AML cells, miR-150 expression levels were also measured in cell samples human patients with AML (Patient IDs 110003417 and 110005410, Conversant Bio, Alabama). FIG. 18 shows miR-150-5p expression levels as percent of total detectable miRs measured in the nanostring experiment. Consistent with FIGS. 17A-17B, miR-150-5p expression was severely reduced in primary and immortalized AML cells compared to healthy immune bone marrow, B cells, macrophages, T cells and monocytes.

### Example 8: AML Cells are Resistant to miR150-Specific Regulation of Protein Expression

As shown in Example 7, all tested AML cells exhibit reduced miR-150 expression. To determine whether miR-150 binding sites could control transfected mRNA protein expression in AML cells, HEL and THP cell lines were transfected with mRNA constructs encoding eGFP reporter proteins.

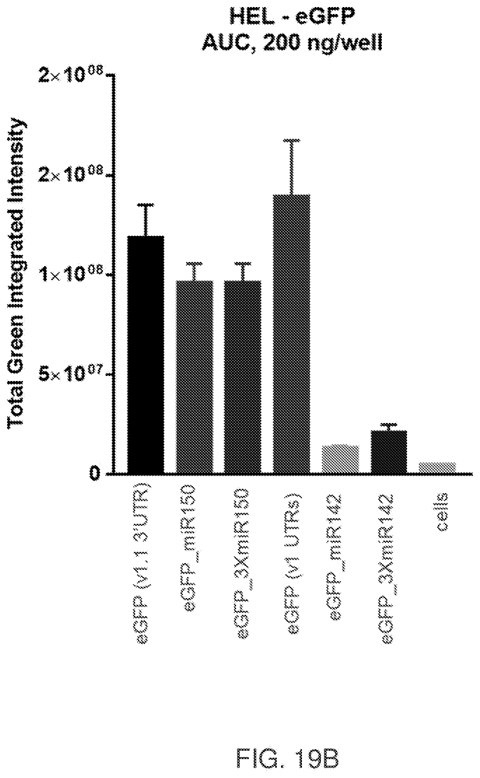

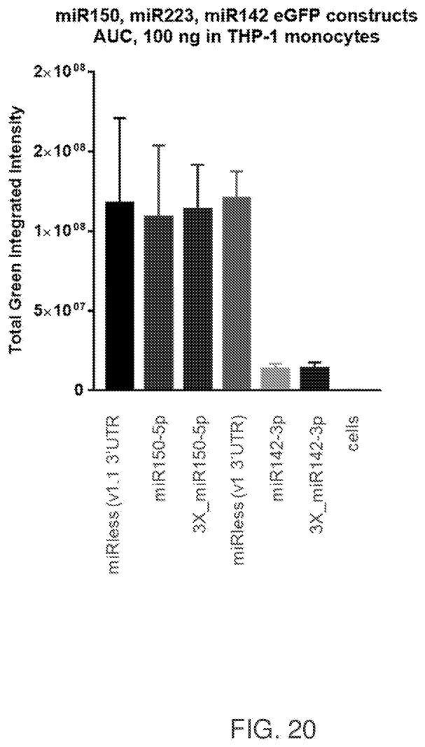

The results show that in AML cells that have demonstrably reduced miR-150 expression, eGFP protein expression remained unchanged after Lipofectamine transfection with eGFP mRNA constructs having either one or three miR-150 binding sites in both HEL cells measured at 24 hours (FIG. 19A) and at 48 hours (FIG. 19B) after transfection and in THP-1 cells measured at 48 hours after transfection (FIG. 20). This dysregulation is not a global phenomenon as transfection with eGFP mRNA having miR-142 binding sites successfully reduced eGFP expression levels (FIGS. 19A, 19B, 20). Notably, miR-142 expression levels were not changed in AML cells compared to normal cells. This data shows that including miR-150-target-sites in mRNA drugs allows selective expression of transfected polypeptide in AML vs. healthy immune cells.

### Example 9: miRs Selectively Expressed in Activated Immune Cells

miR expression in different T cells are measured to determine if certain miRs are selectively upregulated in certain T cell compartments. T cell populations: regulatory, naïve, and effector were isolated from human PBMCs by fluorescence activated cell sorting (FACS). Cells were characterized using the following gating strategy:

Regulatory T cells: CD3(BV421)+CD4(FITC)+CD8(BV605)− CD45RA(AF700)+

CD45RO(APC)− CD25(PE)+CD127(PECy7)low;

Naive T cells: CD3(BV421)+CD4(FITC)+CD8(BV605)− CD45RA(AF700)+

CD45RO(APC)− CD25(PE)− CD127(PECy7)+

Effector T cells: CD3(BV421)+CD4(FITC)+CD8(BV605)− CD45RA(AF700)−

CD45RO(APC)+CD25(PE)+CD127(PECy7)−

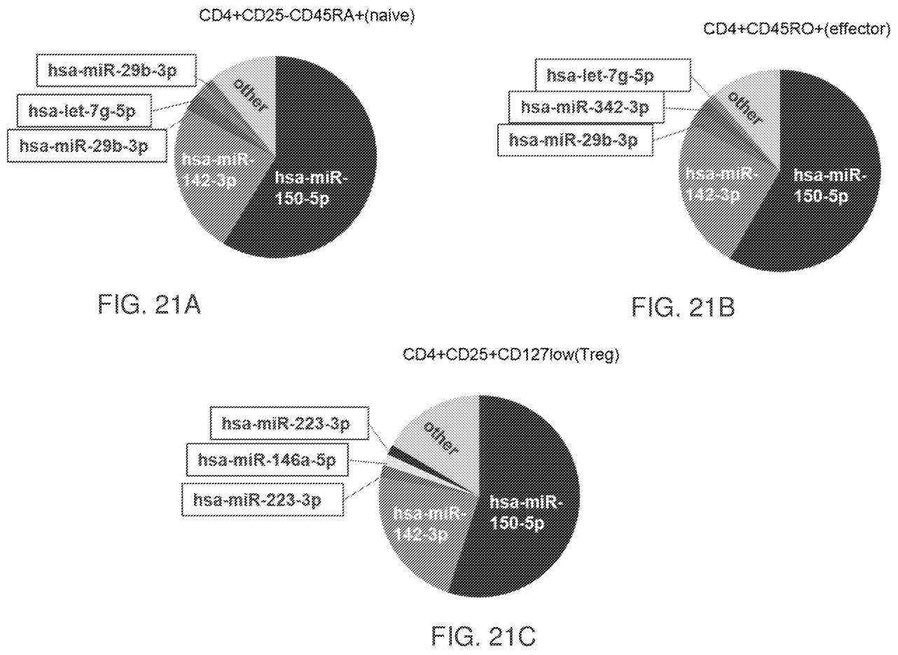

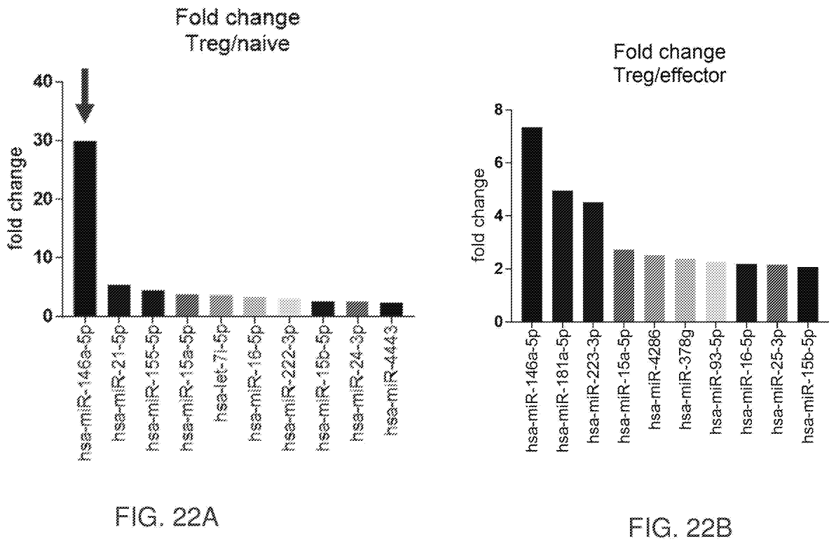

Total mRNAs were extracted from each cell population using phenol chloroform extraction and purification using Qiagen miRNeasy Mini Kit (Qiagen, cat #217004). One hundred nanograms of RNA from each cell population was loaded for analysis using the Nanostring human miR panel (NS_H_MIR_V3A, Nanostring, Washington). miR expression as a fraction of total expressed miRs are summarized in the pie charts for each cell population as shown in FIGS. 21A-21C. miR150-5p, followed by miR142-3p, represent the biggest fraction of miRs expressed in these three cell compartments. The differential miR expression between the cell fractions, Treg/naive and Treg/effector, was calculated and represented in the bar charts shown in FIG. 22A and FIG. 22B respectively. Although miR150-5p and miR142-3p represent the biggest fractions of miR in each cell compartment, the levels remain high and unchanged for each cell type as shown in FIGS. 21A-21C. In contrast, miR-146a-5p expression was not detected in either naïve or effector CD4 T cells, but was expressed in T reg cells. The expression of miR-146a-5p in Treg cells are approximately 30- and 7.5-fold increase over naïve and effector T cells, respectively as shown in FIGS. 22A-22B.

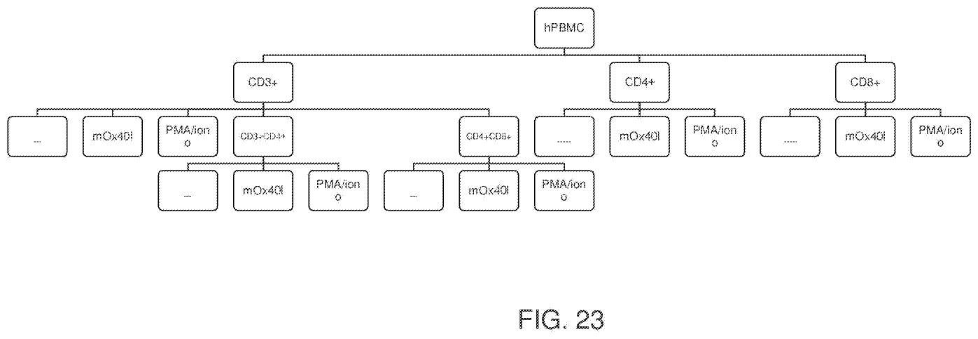

miR expression in activated T cells are measured to determine if certain miRs are selectively upregulated in activated T cells. T cells were isolated from human PBMCs using EasySep Human CD3 Positive Selection Kit II (Stemcell Technologies, Cat 17851) and were cultured under activating conditions, or further isolated using EasySep Human CD4+ T Cell Isolation Kit (Stemcell, Cat 17952) or EasySep Human CD8+ T Cell Isolation Kit (Stemcell, Cat 17953) and then cultured under activating conditions, either by transfection with mOX40L mRNA (1 μg) or with a 1×PMA/ionomycin cocktail mixture (Thermo Fisher, cat. no. 00-4970-03), to evaluate induction of miR expression in mOX40L mRNA transfected or PMA/ionomycin activated cells, as shown in the experimental design depicted in FIG. 23. Total mRNAs were extracted from each cell population using phenol chloroform extraction and purification using Qiagen miRNeasy Mini Kit (Qiagen, cat #217004). One hundred nanograms of RNA from each cell population was loaded for analysis using the Nanostring human miR panel (NS_H_MIR_V3A. Nanostring, Washington).

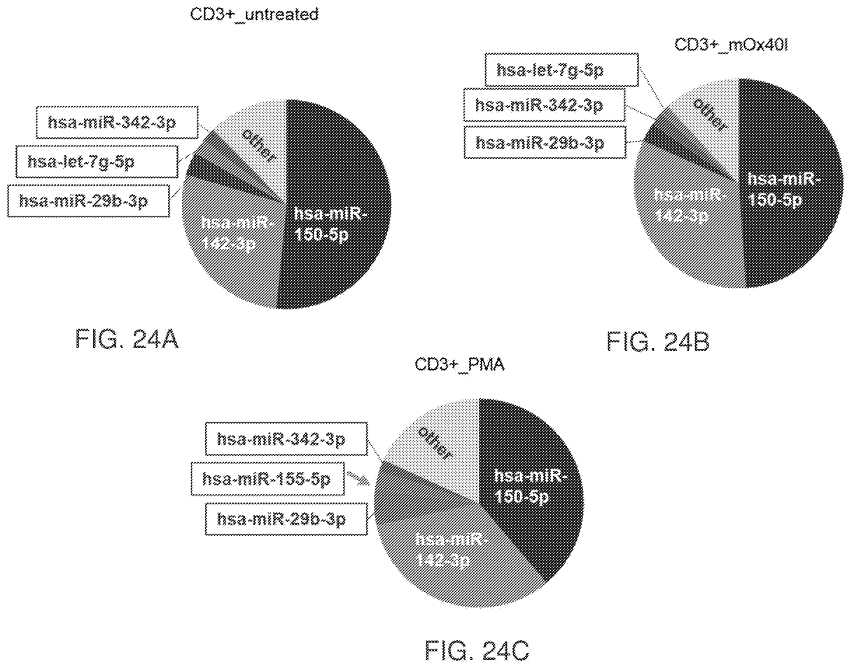

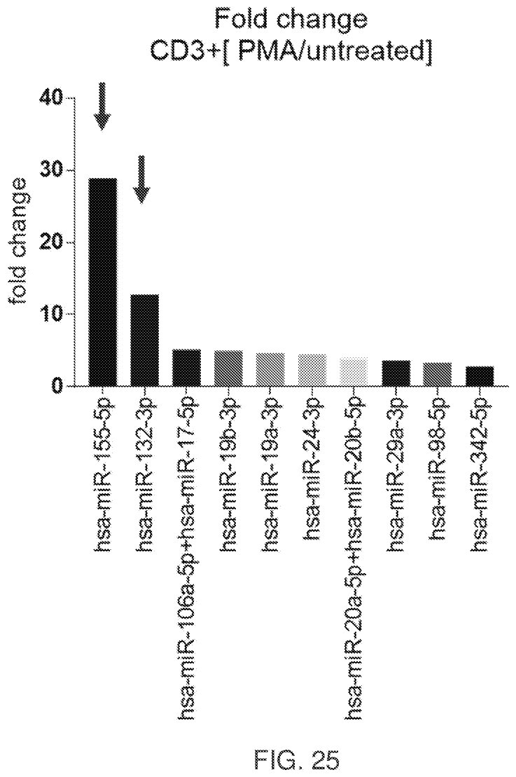

miR expression pattern in untreated and mOX40L mRNA transfected CD3+ cells are shown in FIGS. 24A-24B. There are no changes in the relative abundance of the three highest expressing miRs, miR-150-5p, miR-142-3p, and miR-29b-3p in untreated, mOX40L and PMA/ionomycin treated cells as shown in FIGS. 24A-24C. Upon PMA/Ionomycin treatment, expression of miR155-5p and miR132-3p increased approximately by 30- and 12-fold in CD3+CD4+ cells as shown in FIG. 24C and FIG. 25.

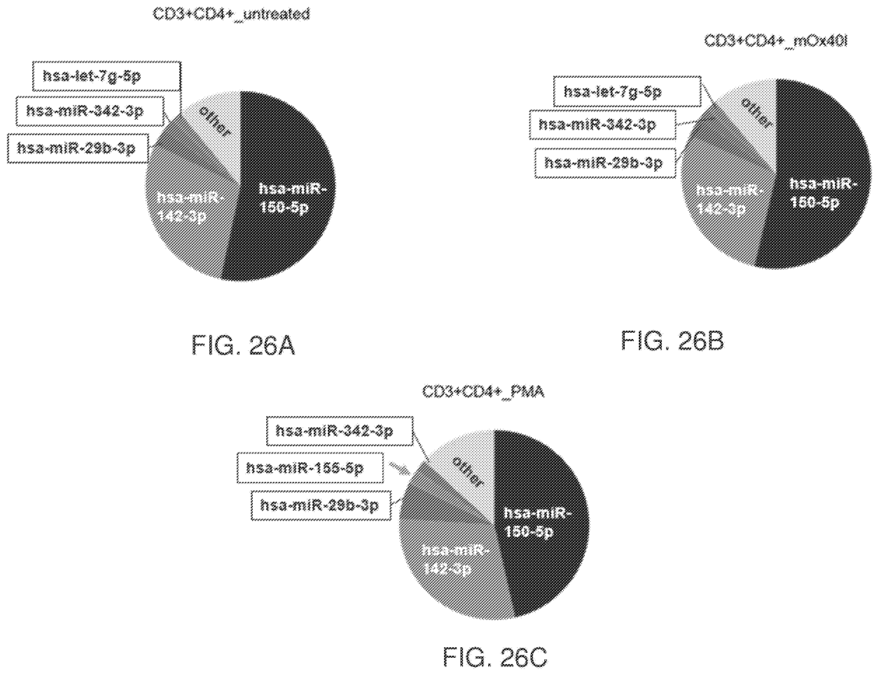

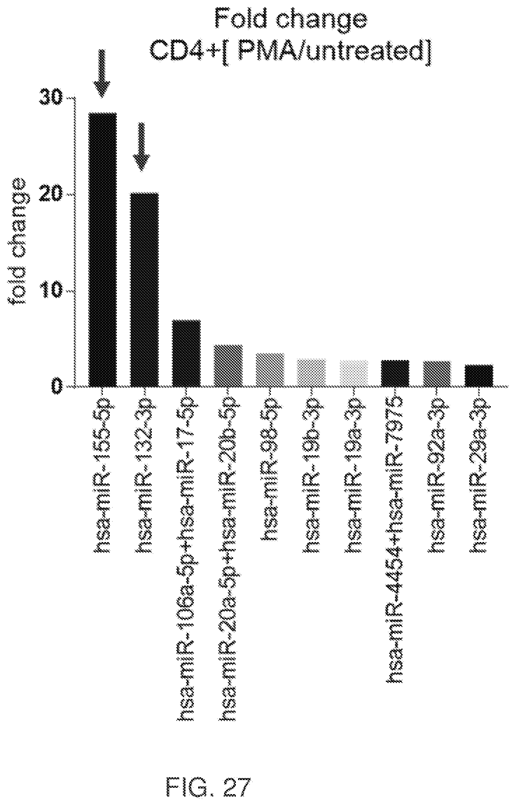

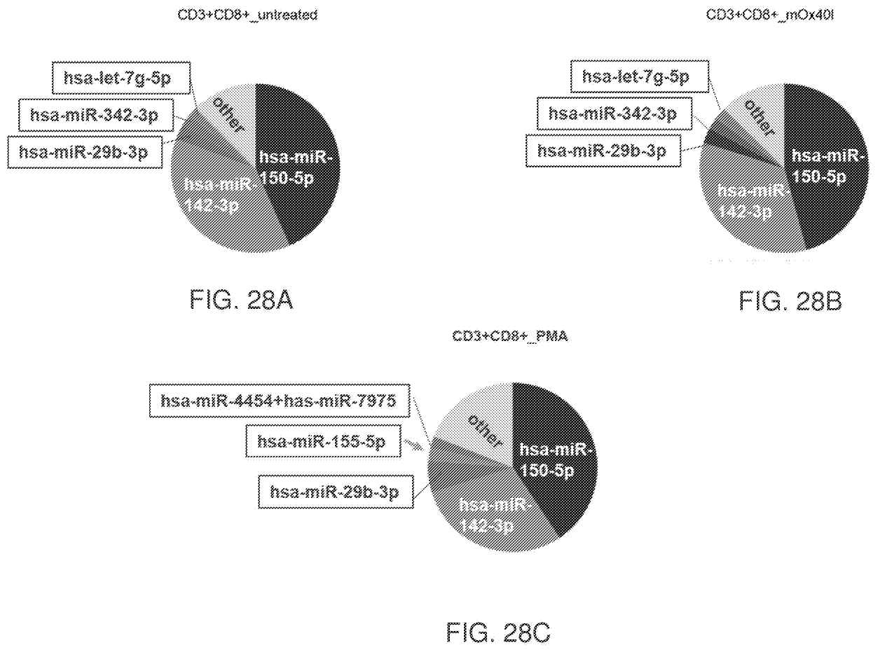

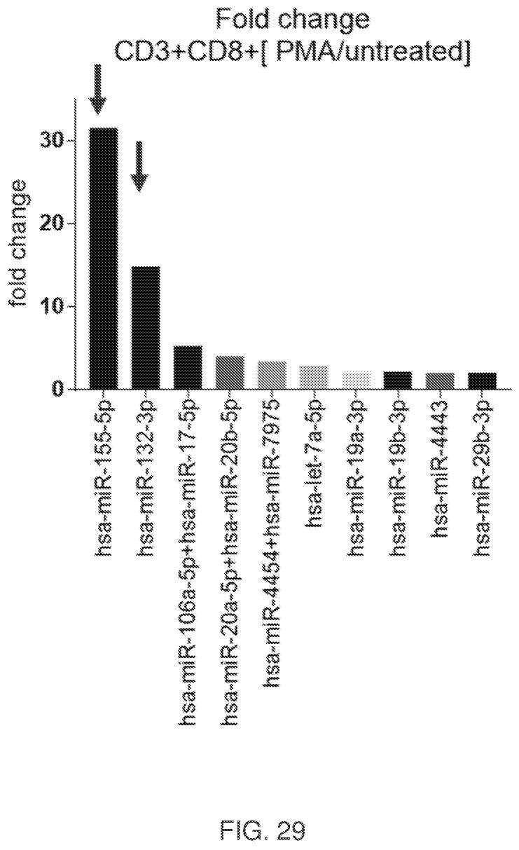

CD3+CD4+ T cells also display a similar change in miR expression profile upon PMA/ionomycin activation, with miR155-5p increasing approximately 29-fold and miR132-3p increasing approximately 19-fold, FIGS. 26A-26C and FIG. 27. The increase in miR155-5p and miR132-3p levels upon PMA/ionomycin activation was also seen in CD3+CD8+ T cells, with miR155-5p increasing approximately 30-fold and miR132-3p increasing approximately 15-fold, FIGS. 28A-28C and FIG. 29. The results here show that miR150-5p and miR142-3p are highly expressed in T cell compartments, with levels relatively unchanged by co-stimulation by mOX40L transfection or PMA/ionomycin activation. However, the expression of miR-155-5p was induced by PMA/ionomycin treatment in all tested T cell populations: CD3+, CD3+CD4+, CD3+CD8+, Treg cells, CD4+ effector T cells, and CD4+ naïve T cells. The selective induction of miR-155-5p cells upon T cell activation also affords another avenue for selectively targeting mRNAs expression in immune cells.

### Example 10: miR-155-5p Expression is Upregulated in Activated PBMCs

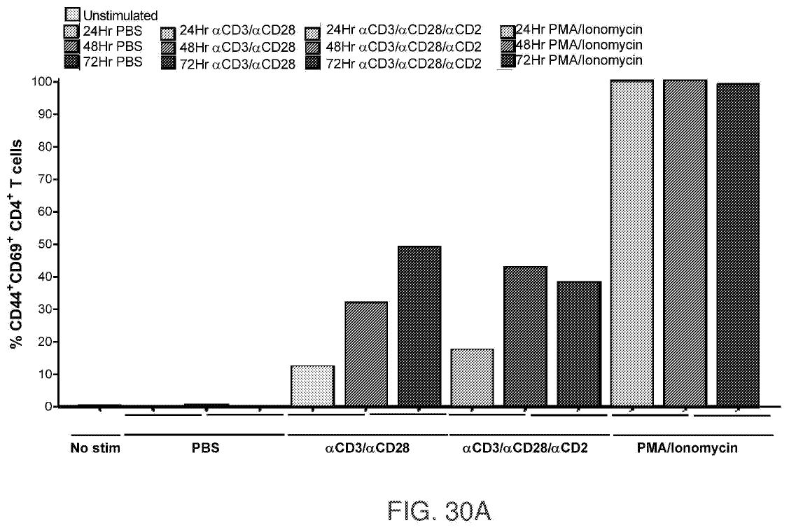

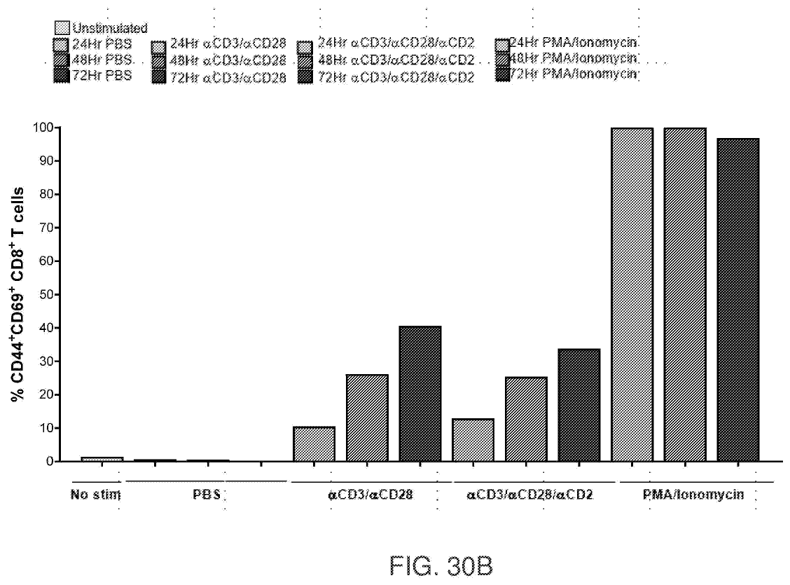

miR-155-5p expression in activated primary T cells is measured in this study. PBMCs from 5 different donors were treated according to the manufacturer's recommendations with (1) anti-CD3 and anti-CD28 antibodies (StemCell 10971), (2) anti-CD3, anti-CD28, and anti-CD2 antibodies (StemCell 10970); or (3) PMA/ionomycin (eBioscience 00-4970-03). Activated T cell numbers were measured at 24, 48, or 72 hours in a FACS assay. The data from one donor PBMC is shown in FIGS. 30A-30B, where all three activation treatments resulted in the increase of activated CD4+ cells (CD44+CD69+ cells) (FIG. 30A) as a percentage of total CD4+ cells at the three tested time points and activated CD8+ cells (CD44+CD69+ cells) (FIG. 30B) as a percentage of total CD8+ cells at the three tested time points. PBMCs from the four other donors showed similar trends (data not shown).

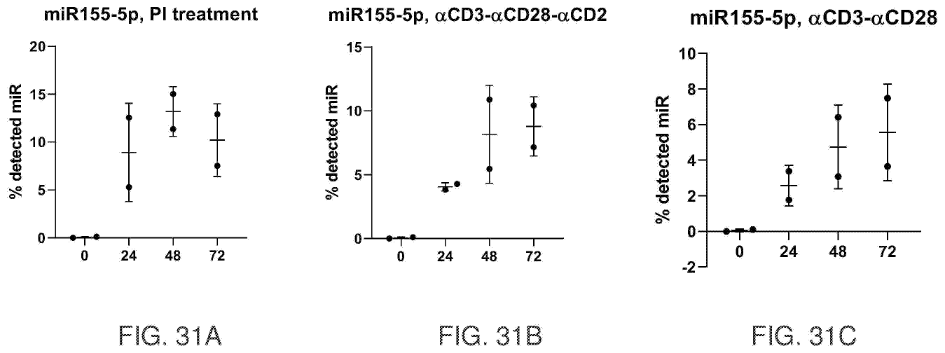

In the next experiment, miR-155-5p expression was measured to determine if miR-155-5p is selectively upregulated in activated T cells. Total mRNAs from 2 donor PBMCs treated as described above were isolated using phenol chloroform extraction and purified using Qiagen miRNeasy Mini Kit (Qiagen, cat #217004). One hundred nanograms of total mRNA was loaded for analysis using the Nanostring human miR panel (NS_H_MIR_V3A, Nanostring, Washington). miR-155-5p expression as a percentage of total detectable miR in the miR panel is summarized in FIGS. 31A-31C. At all time points tested, PBMCs activated by all three treatment groups showed increased expression of miR-155-5p relative to total detectable miRs.

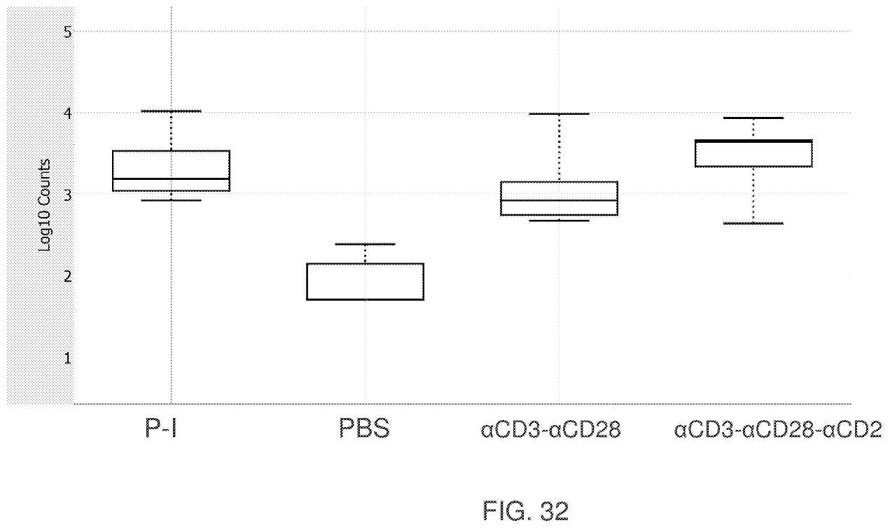

miR-155 expression at 24 hours after initiation of treatments was further studied in FIG. 32. In all five donor PBMCS, miR-155-5p expression at 24-hours showed an approximately 10-fold increase for all three activation treatments compared to PBS.

The upregulation of miR-155-5p expression in T cells after exposure to activation signals presents an opportunity to regulate activation-state dependent mRNA expression in immune cells.

### Example 11: Incorporation of miR-155-5p Binding Sites in mRNA Downregulates Expression of Encoded Protein Expression in Activated T Cells

The effect of incorporating miR-155 binding sites into mRNA construct on the expression of encoded protein was evaluated in PBMCs. mRNA constructs encoding mOX40L and comprising either zero (miRless), one—(hmiR155), or three—(3× hmiR155) miR-155 binding sites in the 3′UTR were tested. The sequence of miR-155-5p and the corresponding miR-155-5p binding sites are UUAAUGCUAAUCGUGAUAGGGGU (SEQ ID NO: 71) and ACCCCTATCACGATTAGCATTAA (SEQ ID NO: 72), respectively. The mRNA constructs were modified by substitution of uracil with N1-methylpseudouracil. The mRNA constructs were encapsulated in lipid nanoparticles comprising compound X/DSPC/cholesterol/beta-sitosterol/PEG DMG at a ratio of 50:10:10:28.5:1.5 and transfected into PANT cells (Stemcell Technologies, Cat 70024). Before mRNA treatment, T cells were cultured under resting (untreated) or activating conditions for three days, either with PMA/ionomycin (1× concentration), or anti-CD3 and anti-CD28 dynabeads (Thermofisher Scientific #11131D) at 1:1 bead:cell ratio.

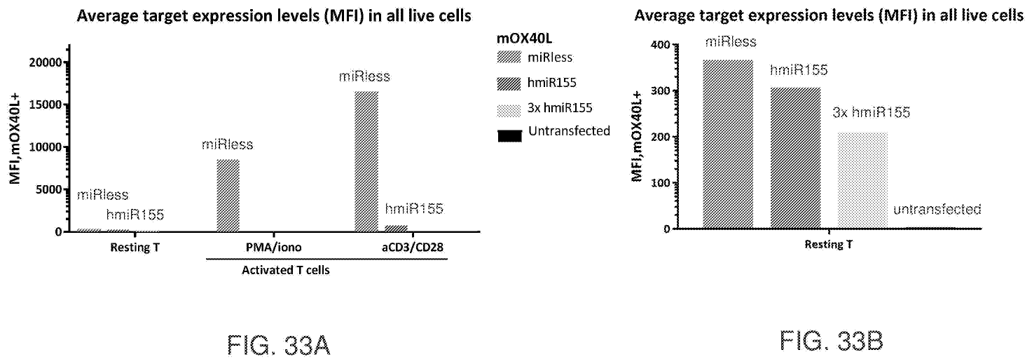

The resting and activated cells were then transfected with lipid nanoparticles encapsulated mRNA, and expression of mOX40L encoded by the transfected mRNA constructs in CD3+ T cells was evaluated by flow cytometry after 24 hours. (FIGS. 33A and 33B). FIG. 33A shows mOX40L expression levels (MFI) in CD3+ cells that were untreated or activated with PMA/ionomycin or anti-CD3/anti-CD28 antibodies. Transfection of mRNA encoding mOX40L resulted in abundant expression of mOX40L protein in activated T cells. Incorporation of one- or three-miR-155 binding site(s) inhibited mOX40L expression in activated T cells. In resting (unstimulated) T cells, transfection of mOX40L encoding mRNA resulted in only modest expression of mOX40L, FIG. 33B. Inclusion of one- or three-miR-155 binding site(s) in the mRNA resulted in a partial decrease of mOX40L expression, approximately 10% and 30%, respectively, in resting T cells.

These results demonstrate that incorporation of miR-155 binding sites into mRNA constructs allows for the selective, i.e., activation-dependent, control of mRNA expression in T cells.

SEQUENCE LISTING

| SEQ ID NO | Description | Sequence |
|---|---|---|
| 1 | miR-150-5p | UCUCCCAACCCUUGUACCAGUG |
| 2 | miR-150-5p | CACTGGTACAAGGGTTGGGAGA |
| | binding site | |
| 3 | miR-146b-5p | UGAGAACUGAAUUCCAUAGGCUG |
| 4 | miR-146b-5p | CAGCCTATGGAATTCAGTTCTCA |
| | binding | |
| | site | |
| 5 | miR 4286 | ACCCCACUCCUGGUACC |
| 6 | miR-4286 | GGTACCAGGAGTGGGGT |
| | binding site | |
| 7 | miR-579-3b | UUCAUUUGGUAUAAACCGCGAUU |
| 8 | miR-579-3b | AATCGCGGTTTATACCAAATGAA |
| | binding site | |
| 9 | miR-4516 | GGGAGAAGGGUCGGGGC |
| 10 | miR-4516 | GCCCCGACCCTTCTCCC |
| | binding site | |
| 11 | miR-146a-5p | UGAGAACUGAAUUCCAUGGGUU |
| 12 | miR-146a-5p | AACCCATGGAATTCAGTTCTCA |
| | binding site | |
| 13 | miR-664b-3p | UUCAUUUGCCUCCCAGCCUACA |
| 14 | miR-664b-3p | TGTAGGCTGGGAGGCAAATGAA |
| | binding site | |
| 15 | miR-342-3p | UCUCACACAGAAAUCGCACCCGU |
| 16 | miR-342-3p | ACGGGTGCGATTTCTGTGTGAGA |
| | binding site | |
| 17 | miR-342-5p | AGGGGUGCUAUCUGUGAUUGA |
| 18 | miR-342-5p | TCAATCACAGATAGCACCCCT |
| | binding site | |
| 19 | miR-1915-3p | CCCCAGGGCGACGCGGCGGG |
| 20 | miR-1915-3p | CCCGCCGCGTCGCCCTGGGG |
| | binding site | |
| 21 | miR-26b-5p | UUCAAGUAAUUCAGGAUAGGU |
| 22 | miR-26b-5p | ACCTATCCTGAATTACTTGAA |
| | binding site | |
| 23 | miR-18a-5p | UAAGGUGCAUCUAGUGCAGAUAG |
| 24 | miR-18a-5p | CTATCTGCACTAGATGCACCTTA |
| | binding site | |
| 25 | miR-1246 | AAUGGAUUUUUGGAGCAGG |
| 26 | miR-1246 | CCTGCTCCAAAAATCCATT |
| | binding site | |
| 27 | miR 126-3p | UCGUACCGUGAGUAAUAAUGCG |
| 28 | miR 126-3p | CGCATTATTACTCACGGTACGA |
| | binding site | |
| 29 | 2A peptide | GSGATNFSLLKQAGDVEENPGP |
| | amino acid | |
| 30 | 2A peptide | GGAAGCGGAGCTACTAACTTCAGCCTGCTGAAGCAGGCT |
| | nucleotide | GGAGACGTGGAGGAGAACCCTGGACCT |
| 31 | 2A peptide | TCCGGACTCAGATCCGGGGATCTCAAAATTGTCGCTCCT |
| | nucleotide | GTCAAACAAACTCTTAACTTTGATTTACTCAAACTGGCT |
| | | GGGGATGTAGAAAGCAATCCAGGTCCACTC |
| 32 | Kozak | GCCA/GCC |
| 33 | V1 | CCCCGGCGCC |
| 34 | V2 | CCCCGGC |
| 35 | EK | GCCGCC |
| 36 | 5′ UTR | GGGAAAUAAGAGAGAAAAGAAGAGUAAGAAGAAAUAUAA |
| | | GA |
| 37 | 5′ UTR | GGGAAAUAAGAGAGAAAAGAAGAGUAAGAAGAAAUAUAA |
| | | GACCCCGGCGCCGCCACC |
| 38 | Standard | GGGAAAUAAGAGAGAAAAGAAGAGUAAGAAGAAAUAUAA |
| | 5′ UTR | GAGCCACC |
| 39 | V1-UTR | GGGAAAUAAGAGAGAAAAGAAGAGUAAGAAGAAAUAUAA |
| | | GACCCCGGCGCCGCCACC |
| 40 | V2-UTR | GGGAAAUAAGAGAGAAAAGAAGAGUAAGAAGAAAUAUAA |
| | | GACCCCGGCGCCACC |
| 41 | Kozak | CCR(A/G)CCAUGG |
| | consensus | where R is A or G |
| 42 | 5′ UTR-001 | GGGAAAUAAGAGAGAAAAGAAGAGUAAGAAGAAAUAUAA |
| | | GAGCCACC |
| 43 | 5′ UTR-002 | GGGAGAUCAGAGAGAAAAGAAGAGUAAGAAGAAAUAUAA |
| | | GAGCCACC |
| 44 | 5′ UTR-004 | GGGAGACAAGCUUGGCAUUCCGGUACUGUUGGUAAAGCC |
| | | ACC |
| 45 | 5′ UTR-005 | GGGAGAUCAGAGAGAAAAGAAGAGUAAGAAGAAAUAUAA |
| | | GAGCCACC |
| 46 | 5′ UTR-007 | GGGAGACAAGCUUGGCAUUCCGGUACUGUUGGUAAAGCC |
| | | ACC |
| 47 | 5′ UTR-008 | GGGAAUUAACAGAGAAAAGAAGAGUAAGAAGAAAUAUAA |
| | | GAGCCACC |
| 48 | 5′ UTR-009 | GGGAAAUUAGACAGAAAAGAAGAGUAAGAAGAAAUAUAA |
| | | GAGCCACC |
| 49 | 5′ UTR-010 | GGGAAAUAAGAGAGUAAAGAACAGUAAGAAGAAAUAUAA |
| | | GAGCCACC |
| 50 | 5′ UTR-011 | GGGAAAAAAGAGAGAAAAGAAGACUAAGAAGAAAUAUAA |
| | | GAGCCACC |
| 51 | 5′ UTR-012 | GGGAAAUAAGAGAGAAAAGAAGAGUAAGAAGAUAUAUAA |
| | | GAGCCACC |
| 52 | 5′ UTR-013 | GGGAAAUAAGAGACAAAACAAGAGUAAGAAGAAAUAUAA |
| | | GAGCCACC |
| 53 | 5′ UTR-014 | GGGAAAUUAGAGAGUAAAGAACAGUAAGUAGAAUUAAAA |
| | | GAGCCACC |
| 54 | 5′ UTR-015 | GGGAAAUAAGAGAGAAUAGAAGAGUAAGAAGAAAUAUAA |
| | | GAGCCACC |
| 55 | 5′ UTR-016 | GGGAAAUAAGAGAGAAAAGAAGAGUAAGAAGAAAAUUAA |
| | | GAGCCACC) |
| 56 | 5′ UTR-017 | GGGAAAUAAGAGAGAAAAGAAGAGUAAGAAGAAAUUUAA |
| | | GAGCCACC |
| 57 | 5′ UTR-018 | UCAAGCUUUUGGACCCUCGUACAGAAGCUAAUACGACUC |
| | | ACUAUAGGGAAAUAAGAGAGAAAAGAAGAGUAAGAAGAA |
| | | AUAUAAGAGCCACC |
| 58 | 3′ UTR with | UGAUAAUAGUCCAUAAAGUAGGAAACACUACAGCUGGAG |
| | miR-142-3p | CCUCGGUGGCCAUGCUUCUUGCCCCUUGGGCCUCCCCCC |
| | binding site | AGCCCCUCCUCCCCUUCCUGCACCCGUACCCCCGUGGUC |
| | | UUUGAAUAAAGUCUGAGUGGGCGGC |
| 59 | 3′ UTR with | UGAUAAUAGGCUGGAGCCUCGGUGGCUCCAUAAAGUAGG |
| | miR-142-3p | AAACACUACACAUGCUUCUUGCCCCUUGGGCCUCCCCCC |
| | binding site | AGCCCCUCCUCCCCUUCCUGCACCCGUACCCCCGUGGUC |
| | | UUUGAAUAAAGUCUGAGUGGGCGGC |
| 60 | 3′ UTR with | UGAUAAUAGGCUGGAGCCUCGGUGGCCAUGCUUCUUGCC |
| | miR-142-3p | CCUUCCAUAAAGUAGGAAACACUACAUGGGCCUCCCCCC |
| | binding site | AGCCCCUCCUCCCCUUCCUGCACCCGUACCCCCGUGGUC |
| | | UUUGAAUAAAGUCUGAGUGGGCGGC |
| 61 | 3′ UTR with | UGAUAAUAGGCUGGAGCCUCGGUGGCCAUGCUUCUUGCC |
| | miR-142-3p | CCUUGGGCCUCCCCCCAGUCCAUAAAGUAGGAAACACUA |
| | binding site | CACCCCUCCUCCCCUUCCUGCACCCGUACCCCCGUGGUC |
| | | UUUGAAUAAAGUCUGAGUGGGCGGC |
| 62 | 3′ UTR with | UGAUAAUAGGCUGGAGCCUCGGUGGCCAUGCUUCUUGCC |
| | miR-142-3p | CCUUGGGCCUCCCCCCAGCCCCUCCUCCCCUUCUCCAUA |
| | binding site | AAGUAGGAAACACUACACUGCACCCGUACCCCCGUGGUC |
| | | UUUGAAUAAAGUCUGAGUGGGCGGC |
| 63 | 3′ UTR with | UGAUAAUAGGCUGGAGCCUCGGUGGCCAUGCUUCUUGCC |
| | miR-142-3p | CCUUGGGCCUCCCCCCAGCCCCUCCUCCCCUUCCUGCAC |
| | binding site | CCGUACCCCCUCCAUAAAGUAGGAAACACUACAGUGGUC |
| | | UUUGAAUAAAGUCUGAGUGGGCGGC |
| 64 | 3′ UTR with | UGAUAAUAGGCUGGAGCCUCGGUGGCCAUGCUUCUUGCC |
| | miR-142-3p | CCUUGGGCCUCCCCCCAGCCCCUCCUCCCCUUCCUGCAC |
| | binding site | CCGUACCCCCGUGGUCUUUGAAUAAAGUUCCAUAAAGU |
| | | AGGAAACACUACACUGAGUGGGCGGC |
| 65 | 3′ UTR-018 | UAAUAGGCUGGAGCCUCGGUGGCCAUGCUUCUUGCCCCU |
| | | UGGGCCUCCCCCCAGCCCCUCCUCCCCUUCCUGCACCCG |
| | | UACCCCCGUGGUCUUUGAAUAAAGUCUGAGUGGGCGGC |
| 66 | 3′ UTR with | UGAUAAUAGUCCAUAAAGUAGGAAACACUACAGCUGGAG |
| | miR-142-3p | CCUCGGUGGCCAUGCUUCUUGCCCCUUGGGCCUCCCCCC |
| | binding site | AGCCCCUCCUCCCCUUCCUGCACCCGUACCCCCCGCAUU |
| | variant 1 | AUUACUCACGGUACGAGUGGUCUUUGAAUAAAGUCUGAG |
| | | UGGGCGGC |
| 67 | 3′ UTR with | UGAUAAUAGUCCAUAAAGUAGGAAACACUACAGCUGGAG |
| | miR-142-3p | CCUCGGUGGCCUAGCUUCUUGCCCCUUGGGCCUCCCCCC |
| | binding site | AGCCCCUCCUCCCCUUCCUGCACCCGUACCCCCCGCAUU |
| | variant 2 | AUUACUCACGGUACGAGUGGUCUUUGAAUAAAGUCUGAG |
| | | UGGGCGGC |
| 68 | 3′ UTR with | UGAUAAUAGGCUGGAGCCUCGGUGGCCUAGCUUCUUGCC |
| | miR-142-3p | CCUUGGGCCUCCCCCCAGCCCCUCCUCCCCUUCCUGCAC |
| | binding site | CCGUACCCCCUCCAUAAAGUAGGAAACACUACAGUGGUC |
| | variant 3 | UUUGAAUAAAGUCUGAGUGGGCGGC |
| 69 | T7 promoter | 5′TAATACGACTCACTATA [symbol] NNNNNNNNN...3′ |
| | sequence | |
| 70 | T7 promoter | 5′TAATACGACTCACTATA [symbol] 3 ′ |
| 71 | miR-155-5p | UUAAUGCUAAUCGUGAUAGGGGU |
| | binding site | |
| 72 | miR-155-5p | ACCCCTATCACGATTAGCATTAA |
| | binding site | |
| 73 | 3′ UTR | TCAAGCTTTTGGACCCTCGTACAGAAGCTAATACGACTC |
| | | ACTATAGGGAAATAAGAGAGAAAAGAAGAGTAAGAAGAA |
| | | ATATAAGAGCCACC |
| 74 | 3′ UTR | TGATAATAGGCTGGAGCCTCGGTGGCCATGCTTCTTGCC |
| | | CCTTGGGCCTCCCCCCAGCCCCTCCTCCCCTTCCTGCAC |
| | | CCGTACCCCCGTGGTCTTTGAATAAAGTCTGAGTGGGCG |
| | | GC |
| 75 | 3′ UTR | TGATAATAGGCTGGAGCCTCGGTGGCCATGCTTCTTGCC |
| | with miR- | CCTTGGGCCCAAACACCATTGTCACACTCCATCCCCCCA |
| | 122 and | GCCCCTCCTCCCCTTCCTCCATAAAGTAGGAAACACTAC |
| | miR-142-3p | ATGCACCCGTACCCCCGTGGTCTTTGAATAAAGTCTGAG |
| | sites | TGGGCGGC |

What is claimed:

1. An mRNA comprising:
(i) a 5′UTR
(ii) an open reading frame (ORF) encoding a polypeptide of interest; and
(iii) a 3′UTR,
wherein the 5′UTR, 3′UTR, or both comprise at least one microRNA (miR)-binding site targeted by a miR,
wherein the miR is differentially expressed in a target human immune cell relative to a plurality of non-target human immune cells, wherein the target human immune cell and the non-target immune cell are selected from (a) different immune cell types, (b) different immune cell states, and (c) different immune cell subpopulations,
such that the mRNA is selectively degraded in the target human immune cell relative to the plurality of non-target human immune cells.

2. The mRNA of claim 1, wherein the target immune cell and the plurality of non-target immune cell are of different immune cell types, optionally between 2 and 5 different immune cell types, optionally greater than 3 different cell types, optionally greater than 4 different cell types.

3. The mRNA of claim 1, wherein the target immune cell and the plurality of non-target immune cell are of different immune cell states, optionally, wherein the plurality of non-target immune cells comprises between 2 and 5 different immune cell states, optionally wherein the plurality of non-target immune cells comprises greater than 3 different cell states, optionally wherein the plurality of non-target immune cells comprises greater than 4 different cell states.

4. The mRNA of claim 3, wherein the target immune cell and the plurality of non-target immune cells are of different activation states.

5. The mRNA of claim 3, wherein the target immune cell and the plurality of non-target immune cells are of different transformation states.

6. The mRNA of claim 1, wherein the target immune cell and the plurality of non-target immune cells are of different immune cell subpopulations.

7. The mRNA of claim 2, wherein the target immune cell is a T cell, and the plurality of non-target immune cells comprises at least two cell types selected from the group consisting of: a dendritic cell (DC), a neutrophil, a natural killer (NK) cell, a monocyte, and a macrophage.

8. The mRNA of claim 7, wherein the miR is selected from the group consisting of: miR-146, miR-23a, miR-142, miR-150, miR-21, and a combination thereof.

9. The mRNA of claim 2, wherein the target immune cell is a DC, and the plurality of non-target immune cells comprises at least two cell types selected from the group consisting of: a T cell, a neutrophil, an NK cell, a monocyte, and a macrophage.

10. The mRNA of claim 9, wherein the miR is selected from the group consisting of: miR-142, miR-223, and a combination miR-142 and miR-223.

11. The mRNA of claim 2, wherein the target immune cell is a neutrophil, and the plurality of non-target immune cells comprises at least two cell types selected from the group consisting of: a T cell, a DC, an NK cell, a monocyte, and a macrophage.

12. The mRNA of claim 11, wherein the miR is selected from the group consisting of: miR-143, miR-23a, miR-142, miR-150, and a combination thereof.

13. The mRNA of claim 2, wherein the target immune cell is an NK cell, and the plurality of non-target immune cells comprises at least two cell types selected from the group consisting of: a T cell, a DC, a neutrophil, a monocyte, and a macrophage.

14. The mRNA of claim 13, wherein the miR is selected from the group consisting of: miR-146, miR-23a, miR-142, miR-223, and a combination thereof.

15. The mRNA of claim 2, wherein the target immune cell is a monocyte, and the plurality of non-target immune cells comprises at least two cell types selected from the group consisting of: a T cell, a DC, a neutrophil, an NK cell, and a macrophage.

16. The mRNA of claim 15, wherein the miR is selected from the group consisting of: miR-23a, miR-142, miR-223, and a combination thereof.

17. The mRNA of claim 2, wherein the target immune cell is a macrophage, and the plurality of non-target immune cells comprises at least two cell types selected from the group consisting of: a T cell, a DC, a neutrophil, an NK cell, and a monocyte.

18. The mRNA of claim 17, wherein the miR is selected from the group consisting of: miR-23a, miR-142, miR-223, and a combination thereof.

19. The mRNA claim 4, wherein the target immune cell is an activated T cell and the miR is differentially expressed in the activated T cell relative to an unstimulated T cell.

20. The mRNA of claim 19, wherein the miR is miR-155a-5p or miR-132-3p.

21. The mRNA claim 5, wherein the target immune cell is a normal immune cell and the miR is differentially expressed in the normal immune cell relative to a cancerous immune cell.

22. The mRNA of claim 21, wherein the normal immune cell is a bone marrow cell, a B cell, a T cell, a monocyte, a macrophage, a dendritic cell or any combination thereof.

23. The mRNA of claim 22, wherein the cancerous immune cell is an AML cell.

24. The mRNA of claim 23, wherein the miR is miR-150-5p, miR-146b-5p, miR-4286, miR-579-3b, miR-4516, miR-146a-5p, miR-664b-3p, miR-342-3p, miR-342-5p, miR-1915-3p, or miR-26b-5p.

25. The mRNA claim 5, wherein the target immune cell is a cancerous immune cell and the miR is differentially expressed in the cancerous immune cell relative to a normal immune cell.

26. The mRNA of claim 25, wherein the normal immune cell is a bone marrow cell, a B cell, a T cell, a monocyte, a macrophage, a dendritic cell or any combination thereof.

27. The mRNA of claim 26, wherein the cancerous immune cell is an AML cell.

28. The mRNA of claim 27, wherein the miR is miR-18a-5p, miR-1246, or miR-126-3p.

29. The mRNA claim 6, wherein the target immune cell is a regulatory T cell and the miR is differentially expressed in regulatory T cells relative to naïve T cells and effector T cells.

30. The mRNA of claim 29, wherein the miR is miR-146a-5p.

31. The mRNA of any one of the preceding claims, wherein the miR is abundantly expressed in the target immune cell relative to the plurality of non-target immune cells.

32. The mRNA of any one of the preceding claims, wherein the polypeptide of interest is selected from a secreted protein, an intracellular protein, a transmembrane, a membrane-bound protein, and a cytotoxic polypeptide.

33. The mRNA of any one of the preceding claims, comprising 2-5 miR-binding sites, two miR-binding sites, three miR-binding sites, or 4 miR-binding sites.

34. The mRNA of any one of the preceding claims, wherein the miR-binding site(s) is located in the 3′ UTR.

35. The mRNA of any one of claims 1-33, wherein the miR-binding site(s) is located in the 5′ UTR.

36. The mRNA of any one of the preceding claims, wherein the mRNA is fully modified with chemically-modified uridines, optionally wherein the chemically-modified uridines comprise N1-methyl-pseudouridine (m1ψ), 5-methoxy-uridine (mo5U), 5-methyl-cytidine (m5C), pseudouridine (ψ), α-thio-guanosine, or α-thio-adenosine, or a combination thereof, optionally wherein the chemically-modified uridines are N1-methylpseudouridines (m1ψ).

37. A lipid nanoparticle (LNP) comprising the mRNA of any one of the preceding claims.

38. The LNP of claim 37 comprising:
(i) an ionizable lipid;
(ii) a sterol or other structural lipid;
(iii) an mRNA molecule of any one of claims 1-36;
(iii) optionally, a non-cationic helper lipid or phospholipid; and
(iv) optionally, a PEG-lipid,
optionally, wherein one or more of (i) the ionizable lipid or (ii) the sterol or other structural lipid comprises an immune cell delivery potentiating lipid in an amount effective to enhance delivery of the LNP to a target immune cell and a plurality of non-target immune cells.

39. A pharmaceutical composition comprising the mRNA of any one of claims 1-36, or the LNP of claim 37 or 38, and a pharmaceutically acceptable carrier.

40. The pharmaceutical composition of claim 39, for use in treating or delaying progression of a disease or disorder in a subject, wherein the treatment comprises administration of the pharmaceutical composition.

41. Use of the pharmaceutical composition of claim 39, in the manufacture of a medicament for treating or delaying progression of a disease or disorder in a subject, wherein the medicament comprises the pharmaceutical composition, and wherein the treatment comprises administration of the medicament.

42. A kit comprising a container comprising the pharmaceutical composition claim 39, and a package insert comprising instructions for administration of the pharmaceutical composition for treating or delaying progression of a disease or disorder in a subject.

43. A method of inducing selective degradation of an mRNA in a target immune cell relative to a plurality of non-target immune cells, comprising contacting a target immune cell with an mRNA of any one of claims 1-36, or an LNP of claim 37 or 38, optionally with a pharmaceutically acceptable carrier, such that the mRNA is selectively degraded in the target immune cell relative to the plurality of non-target immune cells.
